(19) United States
(12) Patent Application Publication
(10) Pub. No.: US 2026/0226526 A1
(43) Pub. Date: Aug. 6, 2026

(54) HOST CELLS CAPABLE OF PRODUCING RETINOL OR RETINOL PRECURSORS AND METHODS OF USE THEREOF

(71) Applicant: AMYRIS, INC., Emeryville, CA (US)

(72) Inventors: Victoria HSIAO, Emeryville, CA (US); Simone M. MANTOVANI, Emeryville, CA (US); William E. DRAPER, Emeryville, CA (US); Anna TAI, Emeryville, CA (US); Yue YANG, Emeryville, CA (US)

(21) Appl. No.: 19/150,515

(22) PCT Filed: Feb. 2, 2024
(86) PCT No.: PCT/US2024/014358

§ 371 Date: Jul. 23, 2025

Related U.S. Application Data
(60) Provisional application No. 63/443,136, filed on Feb. 3, 2023.

(51) Int. Cl. C12P 23/00; C12N 9/10; C12N 9/90; C12P 5/00; C12P 7/22; C12P 7/24; C12P 7/6427

(52) U.S. Cl. CPC C12P 23/00; C12N 9/1085; C12N 9/90; C12P 5/007; C12P 7/22; C12P 7/24; C12P 7/6427; C12Y 205/01029; C12Y 505/01019

(57) ABSTRACT

Provided herein are recombinant host cells, compositions, and methods for the production of retinol, retinal, beta-carotene, lycopene, or phytoene (retinol or retinol precursor). The host cells are genetically modified to contain heterologous nucleic acids that express novel enzymes that enable the host cell to produce the retinol or retinol precursor from a carbon source such as sucrose. The host cells, compositions, and methods disclosed herein provide an efficient route for the heterologous production of retinol, retinal, beta-carotene, lycopene, or phytoene.

### CROSS-REFERENCE TO RELATED APPLICATION

The present application claims the benefit of U.S. Provisional Application No. 63/443,136, filed Feb. 3, 2023, the contents of which is hereby incorporated by reference in its entirety.

### SEQUENCE LISTING

The application contains a Sequence Listing which has been submitted electronically in .XML format and is hereby incorporated by reference in its entirety. Said. XML copy, created on Jan. 31, 2024, is named “107345.00937.xml” and is 573,399 bytes in size. The sequence listing contained in this .XML file is part of the specification and is hereby incorporated by reference herein in its entirety.

### FIELD OF THE INVENTION

The present disclosure relates to recombinant host cells that produce retinol, lycopene, beta-carotene, retinal, or phytoene, and methods of producing these molecules using the recombinant host cells.

### BACKGROUND

Retinoids are a class of lipophilic isoprenoids that are chemically related to vitamin A. Retinol is the most studied and clinically validated cosmetic active beauty ingredient available without a prescription. It increases collagen and elastin production, which can reduce the appearance of fine lines and wrinkles and provide a plump appearance. It helps unclog pores, which can alleviate acne. Finally, retinol has an exfoliating effect that can fade dark spots and improve skin texture and tone.

Retinoids may be synthesized chemically, obtained from animal sources, or produced by genetically modified host organisms. Challenges exist in all of these existing processes, however. For example, microbial host cell production of retinol may lead to co-production of unwanted side products, such as farnesol. Farnesol is a skin irritant and many consumers are resistant to purchasing farnesol-containing products. Thus, co-production of farnesol results in lower yields of the desired retinol product as well as increased downstream purification costs, as retinol and farnesol have similar structures and physical properties, making them very difficult to separate. In addition, retinol is unstable and must be formulated with an antioxidant to prevent oxidation. Many formulations currently use butylated hydroxytoluene (BHT) or butylated hydroxyanisole (BHA) as antioxidants, but there is increasing negative consumer perception of these synthetic additives. Accordingly, there is a need for efficient methods of producing and purifying retinoids, such as retinol, as well as clean, natural means of stabilizing them against oxidation.

### SUMMARY OF THE INVENTION

Provided herein are recombinant host cells that produce retinol, lycopene, beta-carotene, retinal, or phytoene, and methods of producing these molecules using the host cells.

In one aspect, the invention provides for a recombinant host cell capable of producing retinol that contains a heterologous nucleic acid that encodes a first polypeptide having a sequence having at least 80, 85, 90, 95, 99, or 100% identity to a sequence selected from SEQ ID NO: 10, SEQ ID NO: 153, SEQ ID NO: 154, SEQ ID NO: 155, SEQ ID NO: 156, and any one of SEQ ID NOs: 217-240; a heterologous nucleic acid that encodes a second polypeptide having a sequence having at least 80, 85, 90, 95, 99, or 100% identity to a sequence selected from SEQ ID NO: 11, SEQ ID NO: 150, SEQ ID NO: 151, SEQ ID NO: 152, and any one of SEQ ID NOs: 158-216; a heterologous nucleic acid that encodes a third polypeptide having a sequence having at least 80, 85, 90, 95, 99, or 100% identity to a sequence selected from SEQ ID NOs: 12 and 55-149; and a heterologous nucleic acid that encodes a fourth polypeptide having a sequence having at least 80, 85, 90, 95, 99, or 100% identity to a sequence selected from SEQ ID NOs: 14-54.

In an embodiment, the first polypeptide has a sequence selected from SEQ ID NO: 10, SEQ ID NO: 153, SEQ ID NO: 154, SEQ ID NO: 155, SEQ ID NO: 156, and any one of SEQ ID NOs: 217-240, wherein the second polypeptide has a sequence selected from SEQ ID NO: 11, SEQ ID NO: 150, SEQ ID NO: 151, SEQ ID NO: 152, and any one of SEQ ID NOs: 158-216, wherein the third polypeptide has a sequence selected from SEQ ID NOs: 55-149, and wherein the fourth polypeptide has a sequence selected from SEQ ID NOs: 14-54.

In another aspect, the invention provides for a recombinant host cell capable of producing lycopene that contains a heterologous nucleic acid that encodes a phytoene synthase, and a heterologous nucleic acid that encodes a polypeptide having a sequence having at least 80, 85, 90, 95, 99, or 100% identity to a sequence selected from SEQ ID NO: 11, SEQ ID NO: 150, SEQ ID NO: 151, SEQ ID NO: 152, and any one of SEQ ID NOs: 158-216.

In an embodiment, the polypeptide has a sequence selected from SEQ ID NO: 11, SEQ ID NO: 150, SEQ ID NO: 151, SEQ ID NO: 152, and any one of SEQ ID NOs: 158-216.

In a further aspect, the invention provides for a recombinant host cell capable of producing beta-carotene containing a heterologous nucleic acid that encodes a first polypeptide having a sequence having at least 80, 85, 90, 95, 99, or 100% identity to a sequence selected from SEQ ID NO: 10, SEQ ID NO: 153, SEQ ID NO: 154, SEQ ID NO: 155, SEQ ID NO: 156, and any one of SEQ ID NOs: 217-240, and containing a heterologous nucleic acid that encodes a second polypeptide having a sequence having at least 80, 85, 90, 95, 99, or 100% identity to a sequence selected from SEQ ID NO: 11, SEQ ID NO: 150, SEQ ID NO: 151, SEQ ID NO: 152, and any one of SEQ ID NOs: 158-216.

In an embodiment, the first polypeptide has a sequence selected from SEQ ID NO: 10, SEQ ID NO: 153, SEQ ID NO: 154, SEQ ID NO: 155, SEQ ID NO: 156, and any one of SEQ ID NOs: 217-240, and the second polypeptide has a sequence selected from SEQ ID NO: 11, SEQ ID NO: 150, SEQ ID NO: 151, SEQ ID NO: 152, and any one of SEQ ID NOs: 158-216.

In one aspect, the invention provides for a recombinant host cell capable of producing retinal that contains a heterologous nucleic acid that encodes a first polypeptide having a sequence having at least 80, 85, 90, 95, 99, or 100% identity to a sequence selected from SEQ ID NO: 10, SEQ ID NO: 153, SEQ ID NO: 154, SEQ ID NO: 155, SEQ ID NO: 156, and any one of SEQ ID NOs: 217-240; a heterologous nucleic acid that encodes a second polypeptide having a sequence having at least 80, 85, 90, 95, 99, or 100% identity to a sequence selected from SEQ ID NO: 11, SEQ ID NO: 150, SEQ ID NO: 151, SEQ ID NO: 152, and any one of SEQ ID NOs: 158-216; and a heterologous nucleic acid that encodes a third polypeptide having a sequence having at least 80, 85, 90, 95, 99, or 100% identity to a sequence selected from SEQ ID NOs: 12 and 55-149.

In an embodiment, the first polypeptide has a sequence selected from SEQ ID NO: 10, SEQ ID NO: 153, SEQ ID NO: 154, SEQ ID NO: 155, SEQ ID NO: 156, and any one of SEQ ID NOs: 217-240, the second polypeptide has a sequence selected from SEQ ID NO: 11, SEQ ID NO: 150, SEQ ID NO: 151, SEQ ID NO: 152, and any one of SEQ ID NOs: 158-216, and the third polypeptide has a sequence selected from SEQ ID NOs: 12 and 55-149.

In one aspect, the invention provides for a recombinant host cell capable of producing phytoene that contains a heterologous nucleic acid that encodes a first polypeptide having a sequence having at least 80, 85, 90, 95, 99, or 100% identity to a sequence selected from SEQ ID NO: 10, SEQ ID NO: 153, SEQ ID NO: 154, SEQ ID NO: 155, SEQ ID NO: 156, and any one of SEQ ID NOs: 217-240.

In a further embodiment, the recombinant host cell further contains one or more heterologous nucleic acids that encode one or more polypeptides having a sequence having at least 80, 85, 90, 95, 99, or 100% identity to a sequence selected from SEQ ID NO: 3, SEQ ID NO: 1, SEQ ID NO: 7, SEQ ID NO: 157, SEQ ID NO: 2, SEQ ID NO: 8, SEQ ID NO: 5, SEQ ID NO: 6, and SEQ ID NO: 4. In another embodiment, the one or more polypeptides have a sequence selected from SEQ ID NO: 3, SEQ ID NO: 1, SEQ ID NO: 7, SEQ ID NO: 157, SEQ ID NO: 2, SEQ ID NO: 8, SEQ ID NO: 5, SEQ ID NO: 6, and SEQ ID NO: 4.

In certain embodiments, the recombinant host cell further comprises a heterologous nucleic acid that encodes a geranylgeranyl diphosphate synthase having a sequence having at least 80, 85, 90, 95, 99, or 100% identity to a sequence selected from SEQ ID NOs: 9 and 289-310.

In certain embodiments, the recombinant host cell further comprises a deletion of at least a portion of a native alcohol dehydrogenase gene. In certain embodiments, the native alcohol dehydrogenase gene has a sequence having at least 80, 85, 90, 95, 99, or 100% identity to SEQ ID NO: 13.

In certain embodiments, the recombinant host cell further comprises an ERG9 gene that is downregulated relative to the parent strain.

In one embodiment, the host cell is a plant cell, a yeast cell, or a bacterial cell. In another embodiment, the host cell is a yeast cell. In yet another embodiment, the host cell is a Saccharomyces cerevisiae cell.

In one aspect, the invention provides for a method of producing retinol involving culturing a population of recombinant host cells disclosed herein in a culture medium comprising a carbon source under conditions suitable for making retinol, optionally providing an overlay, and recovering the retinol from the culture medium or the overlay.

In an additional aspect, the invention provides for a method of producing lycopene involving culturing a population of recombinant host cells disclosed herein in a culture medium comprising a carbon source under conditions suitable for making lycopene, optionally providing an overlay, and recovering the lycopene from the culture medium or the overlay.

In a further aspect, the invention provides for a method of producing beta-carotene involving culturing a population of recombinant host cells disclosed herein in a culture medium comprising a carbon source under conditions suitable for making beta-carotene, optionally providing an overlay, and recovering the beta-carotene from the culture medium or the overlay.

In yet an additional aspect, the invention provides for a method of producing retinal involving culturing a population of recombinant host cells disclosed herein in a culture medium comprising a carbon source under conditions suitable for making retinal, optionally providing an overlay, and recovering the retinal from the culture medium or the overlay.

In yet additional aspect, the invention provides for a method of producing phytoene involving culturing a population of recombinant host cells disclosed herein in a culture medium comprising a carbon source under conditions suitable for making phytoene, optionally providing an overlay, and recovering the phytoene from the culture medium or the overlay.

### BRIEF DESCRIPTION OF THE FIGURES

FIG. 1 is a graphic representation of the biosynthetic pathway from farnesyl pyrophosphate (FPP) to retinol.

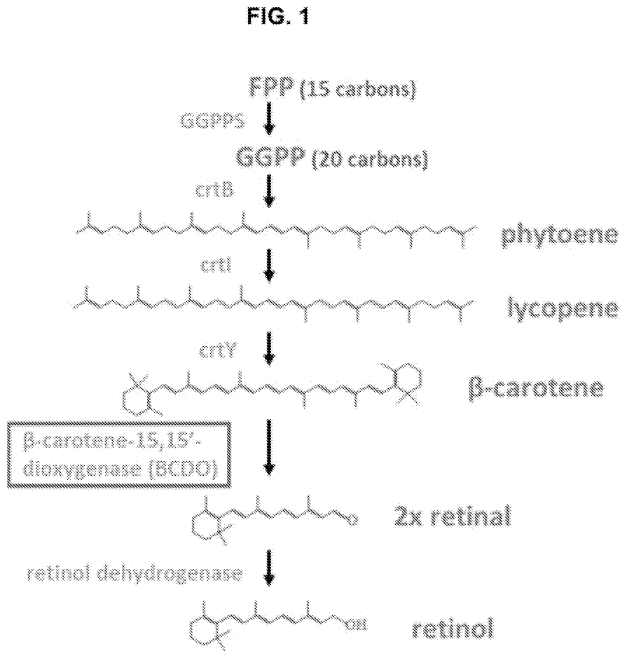

FIG. 2 is a chart showing the percent conversion of retinal into retinol of strains expressing a unique retinal dehydrogenase enzyme.

FIG. 3 is a chart showing the percent improvement in retinol titers over a control strain for strains expressing a unique beta-carotene-15-15′-dioxygenase (BCDO) enzyme.

FIG. 4A is a chart showing retinol titers (mg/L) of strains expressing a unique phytoene desaturase enzyme.

FIG. 4B is a chart showing median retinol titer normalized to the parent strain of strains expressing a unique phytoene desaturase enzyme from the CrtI library in Example 11. The parent strain did not contain the phytoene desaturase enzyme.

FIG. 4C is a chart showing median raw retinol titer (absorbance) of strains expressing a unique phytoene desaturase enzyme from the CrtI library in Example 11. The parent strain did not contain the phytoene desaturase enzyme.

FIG. 5A is a chart showing retinal titers (mg/L) of strains expressing a unique bi-functional phytoene synthase/lycopene cyclase enzyme.

FIG. 5B is a chart showing median retinol titer normalized to the parent strain of strains expressing a unique bi-functional phytoene synthase/lycopene cyclase enzyme from the CrtYB library in Example 13. The parent strain did not contain the phytoene synthase/lycopene cyclase enzyme.

FIG. 5C is a chart showing median raw retinol titer (absorbance) of strains expressing a unique bi-functional phytoene synthase/lycopene cyclase enzyme from the CrtYB library in Example 13. The parent strain did not contain the phytoene synthase/lycopene cyclase enzyme.

FIG. 6 is a chart showing percent conversion of lycopene to beta-carotene for a monofunctional lycopene cyclase (CrtY) biodiversity library.

FIG. 7 is a chart showing retinol titer normalized to the parent strain for a GGPPS biodiversity library.

### DETAILED DESCRIPTION OF THE EMBODIMENTS

### Definitions

As used herein, the term “about” refers to a reasonable range about a value as determined by the practitioner of skill. In certain embodiments, the term about refers to ±one, two, or three standard deviations. In certain embodiments, the term about refers to ±5%, 10%, 20%, or 25%. In certain embodiments, the term about refers to ±0.1, 0.2, or 0.3 logarithmic units, e.g. pH units.

As used herein the terms “comprising,” “including,” “containing,” and “characterized by” are inclusive or open-ended and does not exclude additional, unrecited elements or method steps.

As used herein, the term “heterologous” refers to what is not normally found in nature. The term “heterologous nucleotide sequence” refers to a nucleotide sequence not normally found in a given cell in nature. As such, a heterologous nucleotide sequence may be: (a) foreign to its host cell (i.e., is “exogenous” to the cell); (b) naturally found in the host cell (i.e., “endogenous”) but present at an unnatural quantity in the cell (i.e., greater or lesser quantity than naturally found in the host cell); or (c) be naturally found in the host cell but positioned outside of its natural locus. In certain embodiments, naturally occurring genomic sequences are modified, e.g. codon-optimized, for example, for use in the organisms provided herein.

As used herein, the term “parent cell” refers to a cell that has an identical genetic background as a genetically modified host cell disclosed herein except that it does not comprise one or more particular genetic modifications engineered into the modified host cell, for example, one or more modifications selected from the group consisting of: heterologous expression of an enzyme of a carotenoid pathway such as CrtB, CrtI, CrtY, CrtYB, BCDO and/or RDH.

As used herein, the term “medium” refers to culture medium and/or fermentation medium.

As used herein, the term “production” generally refers to an amount of retinol or retinol precursor produced by a recombinant host cell provided herein. In some embodiments, production is expressed as a yield of retinol or retinol precursor by the host cell. In other embodiments, production is expressed as the productivity of the host cell in producing the retinol or retinol precursor.

As used herein, the term “yield” refers to production of a retinol or retinol precursor by a host cell, expressed as the amount of retinol or retinol precursor produced per amount of carbon source consumed by the host cell, by weight.

As used herein, the term “productivity” refers to production of retinol or retinol precursor by a host cell, expressed as the amount of retinol or retinol precursor produced (by weight) per amount of fermentation broth in which the host cell is cultured (by volume) over time (per hour).

As used herein, the term “recombinant host cell” refers to a host cell that has been genetically modified to express one or more heterologous amino acids that make the host cell capable of producing a particular retinol or retinol precursor. The terms “recombinant host cell,” “host cell,” and “genetically modified host cell” may be used interchangeably to refer to the host cells of the invention.

As used herein, the term “retinol or retinol precursor” refer to a class of isoprenoids that are in the biochemical pathway of retinol synthesis from GGPP. In particular the retinol or retinol precursor of the invention include retinol, retinal, beta-carotene, lycopene, and phytoene.

As used herein, the term “retinol” refers to an isoprenoid that is also known as vitamin A1 and which has the following structure:

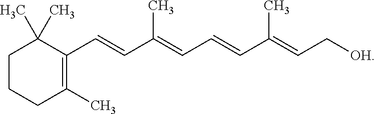 [SMILES: CC1=C(/C=C/C(C)=C/C=C/C(C)=C/CO)C(C)(C)CCC1]

As used herein, the term “retinal” refers to an isoprenoid that is also known as (2E,4E,6E,8E)-3,7-Dimethyl-9-(2,6,6-trimethylcyclohex-1-en-1-yl) nona-2,4,6,8-tetraenal and as vitamin A aldehyde and which has the following structure:

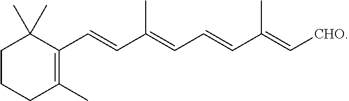 [SMILES: CC1=C(/C=C/C(C)=C/C=C/C(C)=C/C=O)C(C)(C)CCC1]

As used herein, the term “beta-carotene” refers to an isoprenoid that is also known as 1,1′-[(1E,3E,5E,7E,9E,11E,13E,15E,17E)-3,7,12,16-Tetramethyloctadeca-1,3,5,7,9,11,13,15,17-nonaene-1,18-diyl]bis(2,6,6-trimethylcyclohex-1-ene) and as provitamin A and which has the following structure:

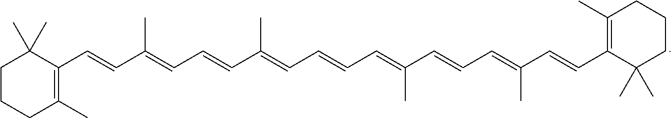 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]

As used herein, the term “lycopene” refers to an isoprenoid that is also known as (6E,8E,10E,12E,14E,16E,18E,20E,22E,24E,26E)-2,6,10,14,19,23,27,31-Octamethyldotriaconta-2,6,8,10,12,14,16,18,20,22,24,26,30-tridecaene and which has the following structure:

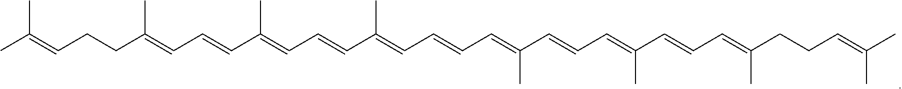 [SMILES: CC(C)=CCC/C(C)=C/C=C/C(C)=C/C=C/C(C)=C/C=C/C=C(C)/C=C/C=C(C)/C=C/C=C(\C)CCC=C(C)C]

As used herein, the term “phytoene” refers to an isoprenoid that is also known as (6E,10E,14E,16Z,18E,22E,26E)-2,6,10,14,19,23,27,31-Octamethyldotriaconta-2,6,10,14,16,18,22,26,30-nonaene and which has the following structure:

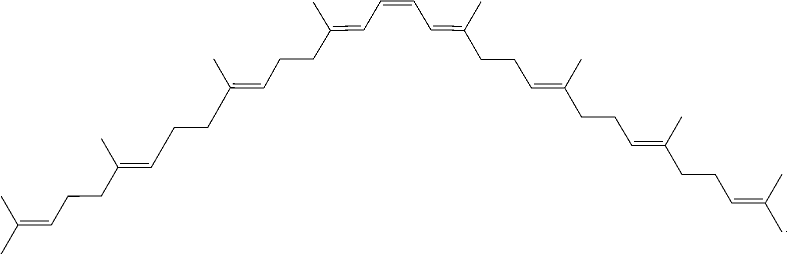 [SMILES: CC(C)=CCC/C(C)=C/CCCC/C(C)=C/CC/C(C)=C/C=C\C=C(/C)CC/C=C(\C)CC/C=C(\C)CCC=C(C)C]

As used herein, the term “sequence identity” or “percent identity” in the context of two or more polynucleotide or polypeptide sequences, refers to two or more sequences or subsequences that are the same or have a specified percentage of nucleotides or amino acid residues that are the same. For example, the sequence may have a percent identity of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or higher identity over a specified region to a reference sequence when compared and aligned for maximum correspondence over a comparison window, or designated region as measured using a sequence comparison algorithm or by manual alignment and visual inspection. For example, percent of identity is determined by calculating the ratio of the number of identical nucleotides (or amino acid residues) in the sequence divided by the length of the total nucleotides (or amino acid residues) minus the lengths of any gaps.

For convenience, the extent of identity between two sequences can be ascertained using computer programs and mathematical algorithms known in the art. Such algorithms that calculate percent sequence identity generally account for sequence gaps and mismatches over the comparison region. Programs that compare and align sequences, like Clustal W (Thompson et al. (1994) Nuclei Acids Res., vol. 22, pp. 4673-4680), ALIGN (Myers et al., (1988) CABIOS, vol. 4, pp. 11-17), FASTA (Pearson et al., (1988) PNAS, vol. 85, pp. 2444-2448; Pearson (1990) Methods Enzymol., vol. 183, pp. 63-98), and gapped BLAST (Altschul et al., (1997) Nucleic Acids Res., vol. 25, pp. 3389-3402) are useful for this purpose. The BLAST or BLAST 2.0 (Altschul et al., (1990) J. Mol. Biol., vol. 215 pp. 403-410) are available from several sources, including the National Center for Biological Information (NCBI) and on the Internet, for use in connection with the sequence analysis programs BLASTP, BLASTN, BLASTX, TBLASTN, and TBLASTX. Additional information can be found at the NCBI web site.

In certain embodiments, the sequence alignments and percent identity calculations can be determined using the BLAST program using its standard, default parameters. For nucleotide sequence alignment and sequence identity calculations, the BLASTN program is used with its default parameters (Gap opening penalty=5, Gap extension penalty=2, Nucleic match=2, Nucleic mismatch=−3, Expectation value=10.0, Word size=11, Max matches in a query range=0). For polypeptide sequence alignment and sequence and sequence identity calculations, BLASTP program is used with its default parameters (Alignment matrix=BLOSUM62; Gap costs: Existence=11, Extension=1; Compositional adjustments=Conditional compositional score, matrix adjustment; Expectation value=10.0; Word size=6; Max matches in a query range=0). Alternatively, the following program and parameters can be used: Align Plus software of Clone Manager Suite, version 5 (Sci-Ed Software); DNA comparison: Global comparison, Standard Linear Scoring matrix, Mismatch penalty=2, Open gap penalty=4, Extend gap penalty=1. Amino acid comparison: Global comparison, BLOSUM 62 Scoring matrix. In the embodiments described herein, the sequence identity is calculated using BLASTN or BLASTP programs using their default parameters. In the embodiments described herein, the sequence alignment of two or more sequences are performed using Clustal W using the suggested default parameters (Dealign input sequences: no; Mbed-like clustering guide-tree: yes; Mbed-like clustering iteration: yes; number of combined iterations: default (0); Max guide tree iterations: default; Max HMM iterations: default; Order: input).

### Production of Retinol and Retinol Precursors by Recombinant Host Cells

In an aspect, the disclosure features a recombinant host cell capable of producing retinol comprising a heterologous nucleic acid that encodes a first polypeptide having a sequence having at least 80, 85, 90, 95, 99, or 100% identity to a sequence selected from SEQ ID NO: 10, SEQ ID NO: 153, SEQ ID NO: 154, SEQ ID NO: 155, SEQ ID NO: 156, and any one of SEQ ID NOs: 217-240; a heterologous nucleic acid that encodes a second polypeptide having a sequence having at least 80, 85, 90, 95, 99, or 100% identity to a sequence selected from SEQ ID NO: 11, SEQ ID NO: 150, SEQ ID NO: 151, SEQ ID NO: 152, and any one of SEQ ID NOs: 158-216; a heterologous nucleic acid that encodes a third polypeptide having a sequence having at least 80, 85, 90, 95, 99, or 100% identity to a sequence selected from SEQ ID NOs: 12 and 55-149; and a heterologous nucleic acid that encodes a fourth polypeptide having a sequence having at least 80, 85, 90, 95, 99, or 100% identity to a sequence selected from SEQ ID NOs: 14-54. In certain embodiments, the recombinant host cell produces retinol.

In another aspect, the disclosure features a recombinant host cell capable of producing retinol comprising a heterologous nucleic acid that encodes a first polypeptide that is a phytoene synthase; a heterologous nucleic acid that encodes a second polypeptide having a sequence having at least 80, 85, 90, 95, 99, or 100% identity to a sequence selected from SEQ ID NO: 11, SEQ ID NO: 150, SEQ ID NO: 151, SEQ ID NO: 152, and any one of SEQ ID NOs: 158-216; a heterologous nucleic acid that encodes a third polypeptide having a sequence having at least 80, 85, 90, 95, 99, or 100% identity to a sequence selected from SEQ ID NOs: 12 and 55-149; and a heterologous nucleic acid that encodes a fourth polypeptide having a sequence having at least 80, 85, 90, 95, 99, or 100% identity to a sequence selected from SEQ ID NOs: 14-54. In certain embodiments, the first polypeptide does not have or has reduced lycopene cyclase activity. In certain embodiments, the recombinant host cell produces retinol.

In a further aspect, the disclosure provides for a recombinant host cell capable of producing beta-carotene comprising a heterologous nucleic acid that encodes a first polypeptide having a sequence having at least 80, 85, 90, 95, 99, or 100% identity to a sequence selected from SEQ ID NO: 10, SEQ ID NO: 153, SEQ ID NO: 154, SEQ ID NO: 155, SEQ ID NO: 156, and SEQ ID NOs: 217-240, and comprising a heterologous nucleic acid that encodes a second polypeptide having a sequence having at least 80, 85, 90, 95, 99, or 100% identity to a sequence selected from SEQ ID NO: 11, SEQ ID NO: 150, SEQ ID NO: 151, SEQ ID NO: 152, and SEQ ID NOs: 158-216. In certain embodiments, the recombinant host cell produces beta-carotene.

In a further aspect, the disclosure provides for a recombinant host cell capable of producing beta-carotene comprising a heterologous nucleic acid that encodes a first polypeptide that is a phytoene synthase, and comprising a heterologous nucleic acid that encodes a second polypeptide having a sequence having at least 80, 85, 90, 95, 99, or 100% identity to a sequence selected from SEQ ID NO: 11, SEQ ID NO: 150, SEQ ID NO: 151, SEQ ID NO: 152, and SEQ ID NOs: 158-216. In certain embodiments, the first polypeptide does not have or has reduced lycopene cyclase activity. In certain embodiments, the recombinant host cell produces beta-carotene.

In another aspect, the disclosure features a recombinant host cell capable of producing retinal comprising a heterologous nucleic acid that encodes a first polypeptide having a sequence having at least 80, 85, 90, 95, 99, or 100% identity to a sequence selected from SEQ ID NO: 10, SEQ ID NO: 153, SEQ ID NO: 154, SEQ ID NO: 155, SEQ ID NO: 156, and SEQ ID NOs: 217-240; a heterologous nucleic acid that encodes a second polypeptide having a sequence having at least 80, 85, 90, 95, 99, or 100% identity to a sequence selected from SEQ ID NO: 11, SEQ ID NO: 150, SEQ ID NO: 151, SEQ ID NO: 152, and SEQ ID NOs: 158-216; and a heterologous nucleic acid that encodes a third polypeptide having a sequence having at least 80, 85, 90, 95, 99, or 100% identity to a sequence selected from SEQ ID NOs: 12 and 55-149. In certain embodiments, the host cell produces retinal.

In another aspect, the disclosure features a recombinant host cell capable of producing retinal comprising a heterologous nucleic acid that encodes a first polypeptide that is a phytoene synthase; a heterologous nucleic acid that encodes a second polypeptide having a sequence having at least 80, 85, 90, 95, 99, or 100% identity to a sequence selected from SEQ ID NO: 11, SEQ ID NO: 150, SEQ ID NO: 151, SEQ ID NO: 152, and SEQ ID NOs: 158-216; and a heterologous nucleic acid that encodes a third polypeptide having a sequence having at least 80, 85, 90, 95, 99, or 100% identity to a sequence selected from SEQ ID NOs: 12 and 55-149. In certain embodiments, the first polypeptide does not have or has reduced lycopene cyclase activity. In certain embodiments, the host cell produces retinal.

In one aspect, the disclosure features a recombinant host cell capable of producing lycopene comprising a heterologous nucleic acid that encodes a first polypeptide that is a phytoene synthase, and comprising a heterologous nucleic acid that encodes a second polypeptide having a sequence having at least 80, 85, 90, 95, 99, or 100% identity to a sequence selected from SEQ ID NO: 11, SEQ ID NO: 150, SEQ ID NO: 151, SEQ ID NO: 152, and SEQ ID NOs: 158-216. In certain embodiments, the first polypeptide does not have or has reduced lycopene cyclase activity. In certain embodiments, the recombinant host cell produces lycopene.

In one aspect, the invention provides for a recombinant host cell capable of producing phytoene that contains a heterologous nucleic acid that encodes a first polypeptide having a sequence having at least 80, 85, 90, 95, 99, or 100% identity to a sequence selected from SEQ ID NO: 10, SEQ ID NO: 153, SEQ ID NO: 154, SEQ ID NO: 155, SEQ ID NO: 156, and any one of SEQ ID NOs: 217-240. In certain embodiments, the recombinant host cell produces phytoene.

In certain embodiments, the first polypeptide has a sequence having at least 80, 85, 90, 95, 99, or 100% identity to a sequence selected from SEQ ID NO: 10, SEQ ID NO: 153, SEQ ID NO: 154, SEQ ID NO: 155, SEQ ID NO: 156, SEQ ID NO: 217, SEQ ID NO: 220, SEQ ID NO: 221, SEQ ID NO: 223, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, and SEQ ID NO: 240. In certain embodiments, the first polypeptide has a sequence having at least 80, 85, 90, 95, 99, or 100% identity to a sequence selected from SEQ ID NO: 10, SEQ ID NO: 153, SEQ ID NO: 154, SEQ ID NO: 155, SEQ ID NO: 156, SEQ ID NO: 220, SEQ ID NO: 221, SEQ ID NO: 225, SEQ ID NO: 226, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, and SEQ ID NO: 240. In certain embodiments, the first polypeptide has a sequence having at least 80, 85, 90, 95, 99, or 100% identity to a sequence selected from SEQ ID NO: 10, SEQ ID NO: 153, SEQ ID NO: 154, SEQ ID NO: 155, and SEQ ID NO: 156. In certain embodiments, the first polypeptide has a sequence having at least 80, 85, 90, 95, 99, or 100% identity to SEQ ID NO: 10. In certain embodiments, the first polypeptide has a sequence having at least 80, 85, 90, 95, 99, or 100% identity to SEQ ID NO: 153. In certain embodiments, the first polypeptide has a sequence having at least 80, 85, 90, 95, 99, or 100% identity to SEQ ID NO: 154. In certain embodiments, the first polypeptide has a sequence having at least 80, 85, 90, 95, 99, or 100% identity to SEQ ID NO: 155. In certain embodiments, the first polypeptide has a sequence having at least 80, 85, 90, 95, 99, or 100% identity to SEQ ID NO: 156. In certain embodiments, the first polypeptide has a sequence having at least 80, 85, 90, 95, 99, or 100% identity to SEQ ID NO: 217. In certain embodiments, the first polypeptide has a sequence having at least 80, 85, 90, 95, 99, or 100% identity to SEQ ID NO: 218. In certain embodiments, the first polypeptide has a sequence having at least 80, 85, 90, 95, 99, or 100% identity to SEQ ID NO: 219. In certain embodiments, the first polypeptide has a sequence having at least 80, 85, 90, 95, 99, or 100% identity to SEQ ID NO: 220. In certain embodiments, the first polypeptide has a sequence having at least 80, 85, 90, 95, 99, or 100% identity to SEQ ID NO: 221. In certain embodiments, the first polypeptide has a sequence having at least 80, 85, 90, 95, 99, or 100% identity to SEQ ID NO: 222. In certain embodiments, the first polypeptide has a sequence having at least 80, 85, 90, 95, 99, or 100% identity to SEQ ID NO: 223. In certain embodiments, the first polypeptide has a sequence having at least 80, 85, 90, 95, 99, or 100% identity to SEQ ID NO: 224. In certain embodiments, the first polypeptide has a sequence having at least 80, 85, 90, 95, 99, or 100% identity to SEQ ID NO: 225. In certain embodiments, the first polypeptide has a sequence having at least 80, 85, 90, 95, 99, or 100% identity to SEQ ID NO: 226. In certain embodiments, the first polypeptide has a sequence having at least 80, 85, 90, 95, 99, or 100% identity to SEQ ID NO: 227. In certain embodiments, the first polypeptide has a sequence having at least 80, 85, 90, 95, 99, or 100% identity to SEQ ID NO: 228. In certain embodiments, the first polypeptide has a sequence having at least 80, 85, 90, 95, 99, or 100% identity to SEQ ID NO: 229. In certain embodiments, the first polypeptide has a sequence having at least 80, 85, 90, 95, 99, or 100% identity to SEQ ID NO: 230. In certain embodiments, the first polypeptide has a sequence having at least 80, 85, 90, 95, 99, or 100% identity to SEQ ID NO: 231. In certain embodiments, the first polypeptide has a sequence having at least 80, 85, 90, 95, 99, or 100% identity to SEQ ID NO: 232. In certain embodiments, the first polypeptide has a sequence having at least 80, 85, 90, 95, 99, or 100% identity to SEQ ID NO: 233. In certain embodiments, the first polypeptide has a sequence having at least 80, 85, 90, 95, 99, or 100% identity to SEQ ID NO: 234. In certain embodiments, the first polypeptide has a sequence having at least 80, 85, 90, 95, 99, or 100% identity to SEQ ID NO: 235. In certain embodiments, the first polypeptide has a sequence having at least 80, 85, 90, 95, 99, or 100% identity to SEQ ID NO: 236. In certain embodiments, the first polypeptide has a sequence having at least 80, 85, 90, 95, 99, or 100% identity to SEQ ID NO: 237. In certain embodiments, the first polypeptide has a sequence having at least 80, 85, 90, 95, 99, or 100% identity to SEQ ID NO: 238. In certain embodiments, the first polypeptide has a sequence having at least 80, 85, 90, 95, 99, or 100% identity to SEQ ID NO: 239. In certain embodiments, the first polypeptide has a sequence having at least 80, 85, 90, 95, 99, or 100% identity to SEQ ID NO: 240.

In certain embodiments, the second polypeptide has a sequence having at least 80, 85, 90, 95, 99, or 100% identity to a sequence selected from SEQ ID NO: 11, SEQ ID NO: 150, SEQ ID NO: 151, SEQ ID NO: 152, SEQ ID NO: 158, SEQ ID NO: 159, SEQ ID NO: 162, SEQ ID NO: 163, SEQ ID NO: 166, SEQ ID NO: 167, SEQ ID NO: 169, SEQ ID NO: 170, SEQ ID NO: 172, SEQ ID NO: 173, SEQ ID NO: 174, SEQ ID NO: 176, SEQ ID NO: 178, SEQ ID NO: 179, SEQ ID NO: 180, SEQ ID NO: 182, SEQ ID NO: 183, SEQ ID NO: 184, SEQ ID NO: 187, SEQ ID NO: 188, SEQ ID NO: 189, SEQ ID NO: 190, SEQ ID NO: 191, SEQ ID NO: 195, SEQ ID NO: 198, SEQ ID NO: 200, SEQ ID NO: 201, SEQ ID NO: 204, SEQ ID NO: 206, SEQ ID NO: 208, SEQ ID NO: 213, SEQ ID NO: 214, and SEQ ID NO: 216. In certain embodiments, the second polypeptide has a sequence having at least 80, 85, 90, 95, 99, or 100% identity to a sequence selected from SEQ ID NO: 11, SEQ ID NO: 150, SEQ ID NO: 151, SEQ ID NO: 152, SEQ ID NO: 159, SEQ ID NO: 162, SEQ ID NO: 166, SEQ ID NO: 169, SEQ ID NO: 170, SEQ ID NO: 178, SEQ ID NO: 180, SEQ ID NO: 182, SEQ ID NO: 183, SEQ ID NO: 184, SEQ ID NO: 187, SEQ ID NO: 188, SEQ ID NO: 189, SEQ ID NO: 190, SEQ ID NO: 191, SEQ ID NO: 195, SEQ ID NO: 198, SEQ ID NO: 200, SEQ ID NO: 206, SEQ ID NO: 208, and SEQ ID NO: 214. In certain embodiments, the second polypeptide has a sequence having at least 80, 85, 90, 95, 99, or 100% identity to a sequence selected from SEQ ID NO: 11, SEQ ID NO: 150, SEQ ID NO: 151, and SEQ ID NO: 152. In certain embodiments, the second polypeptide has a sequence having at least 80, 85, 90, 95, 99, or 100% identity to SEQ ID NO: 11. In certain embodiments, the second polypeptide has a sequence having at least 80, 85, 90, 95, 99, or 100% identity to SEQ ID NO: 150. In certain embodiments, the second polypeptide has a sequence having at least 80, 85, 90, 95, 99, or 100% identity to SEQ ID NO: 151. In certain embodiments, the second polypeptide has a sequence having at least 80, 85, 90, 95, 99, or 100% identity to SEQ ID NO: 152. In certain embodiments, the second polypeptide has a sequence having at least 80, 85, 90, 95, 99, or 100% identity to SEQ ID NO: 158. In certain embodiments, the second polypeptide has a sequence having at least 80, 85, 90, 95, 99, or 100% identity to SEQ ID NO: 159. In certain embodiments, the second polypeptide has a sequence having at least 80, 85, 90, 95, 99, or 100% identity to SEQ ID NO: 160. In certain embodiments, the second polypeptide has a sequence having at least 80, 85, 90, 95, 99, or 100% identity to SEQ ID NO: 161. In certain embodiments, the second polypeptide has a sequence having at least 80, 85, 90, 95, 99, or 100% identity to SEQ ID NO: 162. In certain embodiments, the second polypeptide has a sequence having at least 80, 85, 90, 95, 99, or 100% identity to SEQ ID NO: 163. In certain embodiments, the second polypeptide has a sequence having at least 80, 85, 90, 95, 99, or 100% identity to SEQ ID NO: 164. In certain embodiments, the second polypeptide has a sequence having at least 80, 85, 90, 95, 99, or 100% identity to SEQ ID NO: 165. In certain embodiments, the second polypeptide has a sequence having at least 80, 85, 90, 95, 99, or 100% identity to SEQ ID NO: 166. In certain embodiments, the second polypeptide has a sequence having at least 80, 85, 90, 95, 99, or 100% identity to SEQ ID NO: 167. In certain embodiments, the second polypeptide has a sequence having at least 80, 85, 90, 95, 99, or 100% identity to SEQ ID NO: 168. In certain embodiments, the second polypeptide has a sequence having at least 80, 85, 90, 95, 99, or 100% identity to SEQ ID NO: 169. In certain embodiments, the second polypeptide has a sequence having at least 80, 85, 90, 95, 99, or 100% identity to SEQ ID NO: 170. In certain embodiments, the second polypeptide has a sequence having at least 80, 85, 90, 95, 99, or 100% identity to SEQ ID NO: 171. In certain embodiments, the second polypeptide has a sequence having at least 80, 85, 90, 95, 99, or 100% identity to SEQ ID NO: 172. In certain embodiments, the second polypeptide has a sequence having at least 80, 85, 90, 95, 99, or 100% identity to SEQ ID NO: 173. In certain embodiments, the second polypeptide has a sequence having at least 80, 85, 90, 95, 99, or 100% identity to SEQ ID NO: 174. In certain embodiments, the second polypeptide has a sequence having at least 80, 85, 90, 95, 99, or 100% identity to SEQ ID NO: 175. In certain embodiments, the second polypeptide has a sequence having at least 80, 85, 90, 95, 99, or 100% identity to SEQ ID NO: 176. In certain embodiments, the second polypeptide has a sequence having at least 80, 85, 90, 95, 99, or 100% identity to SEQ ID NO: 177. In certain embodiments, the second polypeptide has a sequence having at least 80, 85, 90, 95, 99, or 100% identity to SEQ ID NO: 178. In certain embodiments, the second polypeptide has a sequence having at least 80, 85, 90, 95, 99, or 100% identity to SEQ ID NO: 179. In certain embodiments, the second polypeptide has a sequence having at least 80, 85, 90, 95, 99, or 100% identity to SEQ ID NO: 180. In certain embodiments, the second polypeptide has a sequence having at least 80, 85, 90, 95, 99, or 100% identity to SEQ ID NO: 181. In certain embodiments, the second polypeptide has a sequence having at least 80, 85, 90, 95, 99, or 100% identity to SEQ ID NO: 182. In certain embodiments, the second polypeptide has a sequence having at least 80, 85, 90, 95, 99, or 100% identity to SEQ ID NO: 183. In certain embodiments, the second polypeptide has a sequence having at least 80, 85, 90, 95, 99, or 100% identity to SEQ ID NO: 184. In certain embodiments, the second polypeptide has a sequence having at least 80, 85, 90, 95, 99, or 100% identity to SEQ ID NO: 185. In certain embodiments, the second polypeptide has a sequence having at least 80, 85, 90, 95, 99, or 100% identity to SEQ ID NO: 186. In certain embodiments, the second polypeptide has a sequence having at least 80, 85, 90, 95, 99, or 100% identity to SEQ ID NO: 187. In certain embodiments, the second polypeptide has a sequence having at least 80, 85, 90, 95, 99, or 100% identity to SEQ ID NO: 188. In certain embodiments, the second polypeptide has a sequence having at least 80, 85, 90, 95, 99, or 100% identity to SEQ ID NO: 189. In certain embodiments, the second polypeptide has a sequence having at least 80, 85, 90, 95, 99, or 100% identity to SEQ ID NO: 190. In certain embodiments, the second polypeptide has a sequence having at least 80, 85, 90, 95, 99, or 100% identity to SEQ ID NO: 191. In certain embodiments, the second polypeptide has a sequence having at least 80, 85, 90, 95, 99, or 100% identity to SEQ ID NO: 192. In certain embodiments, the second polypeptide has a sequence having at least 80, 85, 90, 95, 99, or 100% identity to SEQ ID NO: 193. In certain embodiments, the second polypeptide has a sequence having at least 80, 85, 90, 95, 99, or 100% identity to SEQ ID NO: 194. In certain embodiments, the second polypeptide has a sequence having at least 80, 85, 90, 95, 99, or 100% identity to SEQ ID NO: 195. In certain embodiments, the second polypeptide has a sequence having at least 80, 85, 90, 95, 99, or 100% identity to SEQ ID NO: 196. In certain embodiments, the second polypeptide has a sequence having at least 80, 85, 90, 95, 99, or 100% identity to SEQ ID NO: 197. In certain embodiments, the second polypeptide has a sequence having at least 80, 85, 90, 95, 99, or 100% identity to SEQ ID NO: 198. In certain embodiments, the second polypeptide has a sequence having at least 80, 85, 90, 95, 99, or 100% identity to SEQ ID NO: 199. In certain embodiments, the second polypeptide has a sequence having at least 80, 85, 90, 95, 99, or 100% identity to SEQ ID NO: 200. In certain embodiments, the second polypeptide has a sequence having at least 80, 85, 90, 95, 99, or 100% identity to SEQ ID NO: 201. In certain embodiments, the second polypeptide has a sequence having at least 80, 85, 90, 95, 99, or 100% identity to SEQ ID NO: 202. In certain embodiments, the second polypeptide has a sequence having at least 80, 85, 90, 95, 99, or 100% identity to SEQ ID NO: 203. In certain embodiments, the second polypeptide has a sequence having at least 80, 85, 90, 95, 99, or 100% identity to SEQ ID NO: 204. In certain embodiments, the second polypeptide has a sequence having at least 80, 85, 90, 95, 99, or 100% identity to SEQ ID NO: 205. In certain embodiments, the second polypeptide has a sequence having at least 80, 85, 90, 95, 99, or 100% identity to SEQ ID NO: 206. In certain embodiments, the second polypeptide has a sequence having at least 80, 85, 90, 95, 99, or 100% identity to SEQ ID NO: 207. In certain embodiments, the second polypeptide has a sequence having at least 80, 85, 90, 95, 99, or 100% identity to SEQ ID NO: 208. In certain embodiments, the second polypeptide has a sequence having at least 80, 85, 90, 95, 99, or 100% identity to SEQ ID NO: 209. In certain embodiments, the second polypeptide has a sequence having at least 80, 85, 90, 95, 99, or 100% identity to SEQ ID NO: 210. In certain embodiments, the second polypeptide has a sequence having at least 80, 85, 90, 95, 99, or 100% identity to SEQ ID NO: 211. In certain embodiments, the second polypeptide has a sequence having at least 80, 85, 90, 95, 99, or 100% identity to SEQ ID NO: 212. In certain embodiments, the second polypeptide has a sequence having at least 80, 85, 90, 95, 99, or 100% identity to SEQ ID NO: 213. In certain embodiments, the second polypeptide has a sequence having at least 80, 85, 90, 95, 99, or 100% identity to SEQ ID NO: 214. In certain embodiments, the second polypeptide has a sequence having at least 80, 85, 90, 95, 99, or 100% identity to SEQ ID NO: 215. In certain embodiments, the second polypeptide has a sequence having at least 80, 85, 90, 95, 99, or 100% identity to SEQ ID NO: 216.

In certain embodiments, the third polypeptide has a sequence having at least 80, 85, 90, 95, 99, or 100% identity to a sequence selected from SEQ ID NOs: 12 and 55-149. In certain embodiments, the third polypeptide has a sequence having at least 80, 85, 90, 95, 99, or 100% identity to a sequence selected from SEQ ID NOs: 55-149. In certain embodiments, the third polypeptide has a sequence having at least 80, 85, 90, 95, 99, or 100% sequence identity to SEQ ID NO: 12. In certain embodiments, the third polypeptide has a sequence having at least 80, 85, 90, 95, 99, or 100% sequence identity to SEQ ID NO: 55. In certain embodiments, the third polypeptide has a sequence having at least 80, 85, 90, 95, 99, or 100% sequence identity to SEQ ID NO: 56. In certain embodiments, the third polypeptide has a sequence having at least 80, 85, 90, 95, 99, or 100% sequence identity to SEQ ID NO: 57. In certain embodiments, the third polypeptide has a sequence having at least 80, 85, 90, 95, 99, or 100% sequence identity to SEQ ID NO: 58. In certain embodiments, the third polypeptide has a sequence having at least 80, 85, 90, 95, 99, or 100% sequence identity to SEQ ID NO: 59. In certain embodiments, the third polypeptide has a sequence having at least 80, 85, 90, 95, 99, or 100% sequence identity to SEQ ID NO: 60. In certain embodiments, the third polypeptide has a sequence having at least 80, 85, 90, 95, 99, or 100% sequence identity to SEQ ID NO: 61. In certain embodiments, the third polypeptide has a sequence having at least 80, 85, 90, 95, 99, or 100% sequence identity to SEQ ID NO: 62. In certain embodiments, the third polypeptide has a sequence having at least 80, 85, 90, 95, 99, or 100% sequence identity to SEQ ID NO: 63. In certain embodiments, the third polypeptide has a sequence having at least 80, 85, 90, 95, 99, or 100% sequence identity to SEQ ID NO: 64. In certain embodiments, the third polypeptide has a sequence having at least 80, 85, 90, 95, 99, or 100% sequence identity to SEQ ID NO: 65. In certain embodiments, the third polypeptide has a sequence having at least 80, 85, 90, 95, 99, or 100% sequence identity to SEQ ID NO: 66. In certain embodiments, the third polypeptide has a sequence having at least 80, 85, 90, 95, 99, or 100% sequence identity to SEQ ID NO: 67. In certain embodiments, the third polypeptide has a sequence having at least 80, 85, 90, 95, 99, or 100% sequence identity to SEQ ID NO: 68. In certain embodiments, the third polypeptide has a sequence having at least 80, 85, 90, 95, 99, or 100% sequence identity to SEQ ID NO: 69. In certain embodiments, the third polypeptide has a sequence having at least 80, 85, 90, 95, 99, or 100% sequence identity to SEQ ID NO: 70. In certain embodiments, the third polypeptide has a sequence having at least 80, 85, 90, 95, 99, or 100% sequence identity to SEQ ID NO: 71. In certain embodiments, the third polypeptide has a sequence having at least 80, 85, 90, 95, 99, or 100% sequence identity to SEQ ID NO: 72. In certain embodiments, the third polypeptide has a sequence having at least 80, 85, 90, 95, 99, or 100% sequence identity to SEQ ID NO: 73. In certain embodiments, the third polypeptide has a sequence having at least 80, 85, 90, 95, 99, or 100% sequence identity to SEQ ID NO: 74. In certain embodiments, the third polypeptide has a sequence having at least 80, 85, 90, 95, 99, or 100% sequence identity to SEQ ID NO: 75. In certain embodiments, the third polypeptide has a sequence having at least 80, 85, 90, 95, 99, or 100% sequence identity to SEQ ID NO: 76. In certain embodiments, the third polypeptide has a sequence having at least 80, 85, 90, 95, 99, or 100% sequence identity to SEQ ID NO: 77. In certain embodiments, the third polypeptide has a sequence having at least 80, 85, 90, 95, 99, or 100% sequence identity to SEQ ID NO: 78. In certain embodiments, the third polypeptide has a sequence having at least 80, 85, 90, 95, 99, or 100% sequence identity to SEQ ID NO: 79. In certain embodiments, the third polypeptide has a sequence having at least 80, 85, 90, 95, 99, or 100% sequence identity to SEQ ID NO: 80. In certain embodiments, the third polypeptide has a sequence having at least 80, 85, 90, 95, 99, or 100% sequence identity to SEQ ID NO: 81. In certain embodiments, the third polypeptide has a sequence having at least 80, 85, 90, 95, 99, or 100% sequence identity to SEQ ID NO: 82. In certain embodiments, the third polypeptide has a sequence having at least 80, 85, 90, 95, 99, or 100% sequence identity to SEQ ID NO: 83. In certain embodiments, the third polypeptide has a sequence having at least 80, 85, 90, 95, 99, or 100% sequence identity to SEQ ID NO: 84. In certain embodiments, the third polypeptide has a sequence having at least 80, 85, 90, 95, 99, or 100% sequence identity to SEQ ID NO: 85. In certain embodiments, the third polypeptide has a sequence having at least 80, 85, 90, 95, 99, or 100% sequence identity to SEQ ID NO: 86. In certain embodiments, the third polypeptide has a sequence having at least 80, 85, 90, 95, 99, or 100% sequence identity to SEQ ID NO: 87. In certain embodiments, the third polypeptide has a sequence having at least 80, 85, 90, 95, 99, or 100% sequence identity to SEQ ID NO: 88. In certain embodiments, the third polypeptide has a sequence having at least 80, 85, 90, 95, 99, or 100% sequence identity to SEQ ID NO: 89. In certain embodiments, the third polypeptide has a sequence having at least 80, 85, 90, 95, 99, or 100% sequence identity to SEQ ID NO: 90. In certain embodiments, the third polypeptide has a sequence having at least 80, 85, 90, 95, 99, or 100% sequence identity to SEQ ID NO: 91. In certain embodiments, the third polypeptide has a sequence having at least 80, 85, 90, 95, 99, or 100% sequence identity to SEQ ID NO: 92. In certain embodiments, the third polypeptide has a sequence having at least 80, 85, 90, 95, 99, or 100% sequence identity to SEQ ID NO: 93. In certain embodiments, the third polypeptide has a sequence having at least 80, 85, 90, 95, 99, or 100% sequence identity to SEQ ID NO: 94. In certain embodiments, the third polypeptide has a sequence having at least 80, 85, 90, 95, 99, or 100% sequence identity to SEQ ID NO: 95. In certain embodiments, the third polypeptide has a sequence having at least 80, 85, 90, 95, 99, or 100% sequence identity to SEQ ID NO: 96. In certain embodiments, the third polypeptide has a sequence having at least 80, 85, 90, 95, 99, or 100% sequence identity to SEQ ID NO: 97. In certain embodiments, the third polypeptide has a sequence having at least 80, 85, 90, 95, 99, or 100% sequence identity to SEQ ID NO: 98. In certain embodiments, the third polypeptide has a sequence having at least 80, 85, 90, 95, 99, or 100% sequence identity to SEQ ID NO: 99. In certain embodiments, the third polypeptide has a sequence having at least 80, 85, 90, 95, 99, or 100% sequence identity to SEQ ID NO: 100. In certain embodiments, the third polypeptide has a sequence having at least 80, 85, 90, 95, 99, or 100% sequence identity to SEQ ID NO: 101. In certain embodiments, the third polypeptide has a sequence having at least 80, 85, 90, 95, 99, or 100% sequence identity to SEQ ID NO: 102. In certain embodiments, the third polypeptide has a sequence having at least 80, 85, 90, 95, 99, or 100% sequence identity to SEQ ID NO: 103. In certain embodiments, the third polypeptide has a sequence having at least 80, 85, 90, 95, 99, or 100% sequence identity to SEQ ID NO: 104. In certain embodiments, the third polypeptide has a sequence having at least 80, 85, 90, 95, 99, or 100% sequence identity to SEQ ID NO: 105. In certain embodiments, the third polypeptide has a sequence having at least 80, 85, 90, 95, 99, or 100% sequence identity to SEQ ID NO: 106. In certain embodiments, the third polypeptide has a sequence having at least 80, 85, 90, 95, 99, or 100% sequence identity to SEQ ID NO: 107. In certain embodiments, the third polypeptide has a sequence having at least 80, 85, 90, 95, 99, or 100% sequence identity to SEQ ID NO: 108. In certain embodiments, the third polypeptide has a sequence having at least 80, 85, 90, 95, 99, or 100% sequence identity to SEQ ID NO: 109. In certain embodiments, the third polypeptide has a sequence having at least 80, 85, 90, 95, 99, or 100% sequence identity to SEQ ID NO: 110. In certain embodiments, the third polypeptide has a sequence having at least 80, 85, 90, 95, 99, or 100% sequence identity to SEQ ID NO: 111. In certain embodiments, the third polypeptide has a sequence having at least 80, 85, 90, 95, 99, or 100% sequence identity to SEQ ID NO: 112. In certain embodiments, the third polypeptide has a sequence having at least 80, 85, 90, 95, 99, or 100% sequence identity to SEQ ID NO: 113. In certain embodiments, the third polypeptide has a sequence having at least 80, 85, 90, 95, 99, or 100% sequence identity to SEQ ID NO: 114. In certain embodiments, the third polypeptide has a sequence having at least 80, 85, 90, 95, 99, or 100% sequence identity to SEQ ID NO: 115. In certain embodiments, the third polypeptide has a sequence having at least 80, 85, 90, 95, 99, or 100% sequence identity to SEQ ID NO: 116. In certain embodiments, the third polypeptide has a sequence having at least 80, 85, 90, 95, 99, or 100% sequence identity to SEQ ID NO: 117. In certain embodiments, the third polypeptide has a sequence having at least 80, 85, 90, 95, 99, or 100% sequence identity to SEQ ID NO: 118. In certain embodiments, the third polypeptide has a sequence having at least 80, 85, 90, 95, 99, or 100% sequence identity to SEQ ID NO: 119. In certain embodiments, the third polypeptide has a sequence having at least 80, 85, 90, 95, 99, or 100% sequence identity to SEQ ID NO: 120. In certain embodiments, the third polypeptide has a sequence having at least 80, 85, 90, 95, 99, or 100% sequence identity to SEQ ID NO: 121. In certain embodiments, the third polypeptide has a sequence having at least 80, 85, 90, 95, 99, or 100% sequence identity to SEQ ID NO: 122. In certain embodiments, the third polypeptide has a sequence having at least 80, 85, 90, 95, 99, or 100% sequence identity to SEQ ID NO: 123. In certain embodiments, the third polypeptide has a sequence having at least 80, 85, 90, 95, 99, or 100% sequence identity to SEQ ID NO: 124. In certain embodiments, the third polypeptide has a sequence having at least 80, 85, 90, 95, 99, or 100% sequence identity to SEQ ID NO: 125. In certain embodiments, the third polypeptide has a sequence having at least 80, 85, 90, 95, 99, or 100% sequence identity to SEQ ID NO: 126. In certain embodiments, the third polypeptide has a sequence having at least 80, 85, 90, 95, 99, or 100% sequence identity to SEQ ID NO: 127. In certain embodiments, the third polypeptide has a sequence having at least 80, 85, 90, 95, 99, or 100% sequence identity to SEQ ID NO: 128. In certain embodiments, the third polypeptide has a sequence having at least 80, 85, 90, 95, 99, or 100% sequence identity to SEQ ID NO: 129. In certain embodiments, the third polypeptide has a sequence having at least 80, 85, 90, 95, 99, or 100% sequence identity to SEQ ID NO: 130. In certain embodiments, the third polypeptide has a sequence having at least 80, 85, 90, 95, 99, or 100% sequence identity to SEQ ID NO: 131. In certain embodiments, the third polypeptide has a sequence having at least 80, 85, 90, 95, 99, or 100% sequence identity to SEQ ID NO: 132. In certain embodiments, the third polypeptide has a sequence having at least 80, 85, 90, 95, 99, or 100% sequence identity to SEQ ID NO: 133. In certain embodiments, the third polypeptide has a sequence having at least 80, 85, 90, 95, 99, or 100% sequence identity to SEQ ID NO: 134. In certain embodiments, the third polypeptide has a sequence having at least 80, 85, 90, 95, 99, or 100% sequence identity to SEQ ID NO: 135. In certain embodiments, the third polypeptide has a sequence having at least 80, 85, 90, 95, 99, or 100% sequence identity to SEQ ID NO: 136. In certain embodiments, the third polypeptide has a sequence having at least 80, 85, 90, 95, 99, or 100% sequence identity to SEQ ID NO: 137. In certain embodiments, the third polypeptide has a sequence having at least 80, 85, 90, 95, 99, or 100% sequence identity to SEQ ID NO: 138. In certain embodiments, the third polypeptide has a sequence having at least 80, 85, 90, 95, 99, or 100% sequence identity to SEQ ID NO: 139. In certain embodiments, the third polypeptide has a sequence having at least 80, 85, 90, 95, 99, or 100% sequence identity to SEQ ID NO: 140. In certain embodiments, the third polypeptide has a sequence having at least 80, 85, 90, 95, 99, or 100% sequence identity to SEQ ID NO: 141. In certain embodiments, the third polypeptide has a sequence having at least 80, 85, 90, 95, 99, or 100% sequence identity to SEQ ID NO: 142. In certain embodiments, the third polypeptide has a sequence having at least 80, 85, 90, 95, 99, or 100% sequence identity to SEQ ID NO: 143. In certain embodiments, the third polypeptide has a sequence having at least 80, 85, 90, 95, 99, or 100% sequence identity to SEQ ID NO: 144. In certain embodiments, the third polypeptide has a sequence having at least 80, 85, 90, 95, 99, or 100% sequence identity to SEQ ID NO: 145. In certain embodiments, the third polypeptide has a sequence having at least 80, 85, 90, 95, 99, or 100% sequence identity to SEQ ID NO: 146. In certain embodiments, the third polypeptide has a sequence having at least 80, 85, 90, 95, 99, or 100% sequence identity to SEQ ID NO: 147. In certain embodiments, the third polypeptide has a sequence having at least 80, 85, 90, 95, 99, or 100% sequence identity to SEQ ID NO: 148. In certain embodiments, the third polypeptide has a sequence having at least 80, 85, 90, 95, 99, or 100% sequence identity to SEQ ID NO: 149.

In certain embodiments, the fourth polypeptide has a sequence having at least 80, 85, 90, 95, 99, or 100% identity to a sequence selected from SEQ ID NOs: 14-54. In certain embodiments, the fourth polypeptide has a sequence having at least 80, 85, 90, 95, 99, or 100% identity to SEQ ID NO: 14. In certain embodiments, the fourth polypeptide has a sequence having at least 80, 85, 90, 95, 99, or 100% identity to SEQ ID NO: 15. In certain embodiments, the fourth polypeptide has a sequence having at least 80, 85, 90, 95, 99, or 100% identity to SEQ ID NO: 16. In certain embodiments, the fourth polypeptide has a sequence having at least 80, 85, 90, 95, 99, or 100% identity to SEQ ID NO: 17. In certain embodiments, the fourth polypeptide has a sequence having at least 80, 85, 90, 95, 99, or 100% identity to SEQ ID NO: 18. In certain embodiments, the fourth polypeptide has a sequence having at least 80, 85, 90, 95, 99, or 100% identity to SEQ ID NO: 19. In certain embodiments, the fourth polypeptide has a sequence having at least 80, 85, 90, 95, 99, or 100% identity to SEQ ID NO: 20. In certain embodiments, the fourth polypeptide has a sequence having at least 80, 85, 90, 95, 99, or 100% identity to SEQ ID NO: 21. In certain embodiments, the fourth polypeptide has a sequence having at least 80, 85, 90, 95, 99, or 100% identity to SEQ ID NO: 22. In certain embodiments, the fourth polypeptide has a sequence having at least 80, 85, 90, 95, 99, or 100% identity to SEQ ID NO: 23. In certain embodiments, the fourth polypeptide has a sequence having at least 80, 85, 90, 95, 99, or 100% identity to SEQ ID NO: 24. In certain embodiments, the fourth polypeptide has a sequence having at least 80, 85, 90, 95, 99, or 100% identity to SEQ ID NO: 25. In certain embodiments, the fourth polypeptide has a sequence having at least 80, 85, 90, 95, 99, or 100% identity to SEQ ID NO: 26. In certain embodiments, the fourth polypeptide has a sequence having at least 80, 85, 90, 95, 99, or 100% identity to SEQ ID NO: 27. In certain embodiments, the fourth polypeptide has a sequence having at least 80, 85, 90, 95, 99, or 100% identity to SEQ ID NO: 28. In certain embodiments, the fourth polypeptide has a sequence having at least 80, 85, 90, 95, 99, or 100% identity to SEQ ID NO: 29. In certain embodiments, the fourth polypeptide has a sequence having at least 80, 85, 90, 95, 99, or 100% identity to SEQ ID NO: 30. In certain embodiments, the fourth polypeptide has a sequence having at least 80, 85, 90, 95, 99, or 100% identity to SEQ ID NO: 31. In certain embodiments, the fourth polypeptide has a sequence having at least 80, 85, 90, 95, 99, or 100% identity to SEQ ID NO: 32. In certain embodiments, the fourth polypeptide has a sequence having at least 80, 85, 90, 95, 99, or 100% identity to SEQ ID NO: 33. In certain embodiments, the fourth polypeptide has a sequence having at least 80, 85, 90, 95, 99, or 100% identity to SEQ ID NO: 34. In certain embodiments, the fourth polypeptide has a sequence having at least 80, 85, 90, 95, 99, or 100% identity to SEQ ID NO: 35. In certain embodiments, the fourth polypeptide has a sequence having at least 80, 85, 90, 95, 99, or 100% identity to SEQ ID NO: 36. In certain embodiments, the fourth polypeptide has a sequence having at least 80, 85, 90, 95, 99, or 100% identity to SEQ ID NO: 37. In certain embodiments, the fourth polypeptide has a sequence having at least 80, 85, 90, 95, 99, or 100% identity to SEQ ID NO: 38. In certain embodiments, the fourth polypeptide has a sequence having at least 80, 85, 90, 95, 99, or 100% identity to SEQ ID NO: 39. In certain embodiments, the fourth polypeptide has a sequence having at least 80, 85, 90, 95, 99, or 100% identity to SEQ ID NO: 40. In certain embodiments, the fourth polypeptide has a sequence having at least 80, 85, 90, 95, 99, or 100% identity to SEQ ID NO: 41. In certain embodiments, the fourth polypeptide has a sequence having at least 80, 85, 90, 95, 99, or 100% identity to SEQ ID NO: 42. In certain embodiments, the fourth polypeptide has a sequence having at least 80, 85, 90, 95, 99, or 100% identity to SEQ ID NO: 43. In certain embodiments, the fourth polypeptide has a sequence having at least 80, 85, 90, 95, 99, or 100% identity to SEQ ID NO: 44. In certain embodiments, the fourth polypeptide has a sequence having at least 80, 85, 90, 95, 99, or 100% identity to SEQ ID NO: 45. In certain embodiments, the fourth polypeptide has a sequence having at least 80, 85, 90, 95, 99, or 100% identity to SEQ ID NO: 46. In certain embodiments, the fourth polypeptide has a sequence having at least 80, 85, 90, 95, 99, or 100% identity to SEQ ID NO: 47. In certain embodiments, the fourth polypeptide has a sequence having at least 80, 85, 90, 95, 99, or 100% identity to SEQ ID NO: 48. In certain embodiments, the fourth polypeptide has a sequence having at least 80, 85, 90, 95, 99, or 100% identity to SEQ ID NO: 49. In certain embodiments, the fourth polypeptide has a sequence having at least 80, 85, 90, 95, 99, or 100% identity to SEQ ID NO: 50. In certain embodiments, the fourth polypeptide has a sequence having at least 80, 85, 90, 95, 99, or 100% identity to SEQ ID NO: 51. In certain embodiments, the fourth polypeptide has a sequence having at least 80, 85, 90, 95, 99, or 100% identity to SEQ ID NO: 52. In certain embodiments, the fourth polypeptide has a sequence having at least 80, 85, 90, 95, 99, or 100% identity to SEQ ID NO: 53. In certain embodiments, the fourth polypeptide has a sequence having at least 80, 85, 90, 95, 99, or 100% identity to SEQ ID NO: 54.

In an embodiment, the first polypeptide has a sequence selected from SEQ ID NO: 10, SEQ ID NO: 153, SEQ ID NO: 154, SEQ ID NO: 155, SEQ ID NO: 156, and any one of SEQ ID NOs: 217-240, and wherein the second polypeptide has a sequence selected from SEQ ID NO: 11, SEQ ID NO: 150, SEQ ID NO: 151, SEQ ID NO: 152, and any one of SEQ ID NOs: 158-216, and wherein the third polypeptide has a sequence selected from SEQ ID NOs: 12 and 55-148, and wherein the fourth polypeptide has a sequence selected from SEQ ID NOs: 14-54. In certain embodiments, the first polypeptide has a sequence selected from SEQ ID NO: 10, SEQ ID NO: 153, SEQ ID NO: 154, SEQ ID NO: 155, SEQ ID NO: 156, SEQ ID NO: 217, SEQ ID NO: 220, SEQ ID NO: 221, SEQ ID NO: 223, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, and SEQ ID NO: 240. In certain embodiments, the first polypeptide has a sequence selected from SEQ ID NO: 10, SEQ ID NO: 153, SEQ ID NO: 154, SEQ ID NO: 155, SEQ ID NO: 156, SEQ ID NO: 220, SEQ ID NO: 221, SEQ ID NO: 225, SEQ ID NO: 226, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, and SEQ ID NO: 240. In certain embodiments, the first polypeptide has the sequence of SEQ ID NO: 10. In certain embodiments, the first polypeptide has the sequence of SEQ ID NO: 153. In certain embodiments, the first polypeptide has the sequence of SEQ ID NO: 154. In certain embodiments, the first polypeptide has the sequence of SEQ ID NO: 155. In certain embodiments, the first polypeptide has the sequence of SEQ ID NO: 156. In certain embodiments, the first polypeptide has the sequence of SEQ ID NO: 217. In certain embodiments, the first polypeptide has the sequence of SEQ ID NO: 218. In certain embodiments, the first polypeptide has the sequence of SEQ ID NO: 219. In certain embodiments, the first polypeptide has the sequence of SEQ ID NO: 220. In certain embodiments, the first polypeptide has the sequence of SEQ ID NO: 221. In certain embodiments, the first polypeptide has the sequence of SEQ ID NO: 222. In certain embodiments, the first polypeptide has the sequence of SEQ ID NO: 223. In certain embodiments, the first polypeptide has the sequence of SEQ ID NO: 224. In certain embodiments, the first polypeptide has the sequence of SEQ ID NO: 225. In certain embodiments, the first polypeptide has the sequence of SEQ ID NO: 226. In certain embodiments, the first polypeptide has the sequence of SEQ ID NO: 227. In certain embodiments, the first polypeptide has the sequence of SEQ ID NO: 228. In certain embodiments, the first polypeptide has the sequence of SEQ ID NO: 229. In certain embodiments, the first polypeptide has the sequence of SEQ ID NO: 230. In certain embodiments, the first polypeptide has the sequence of SEQ ID NO: 231. In certain embodiments, the first polypeptide has the sequence of SEQ ID NO: 232. In certain embodiments, the first polypeptide has the sequence of SEQ ID NO: 233. In certain embodiments, the first polypeptide has the sequence of SEQ ID NO: 234. In certain embodiments, the first polypeptide has the sequence of SEQ ID NO: 235. In certain embodiments, the first polypeptide has the sequence of SEQ ID NO: 236. In certain embodiments, the first polypeptide has the sequence of SEQ ID NO: 237. In certain embodiments, the first polypeptide has the sequence of SEQ ID NO: 238. In certain embodiments, the first polypeptide has the sequence of SEQ ID NO: 239. In certain embodiments, the first polypeptide has the sequence of SEQ ID NO: 240.

In certain embodiments, the second polypeptide has a sequence selected from SEQ ID NO: 11, SEQ ID NO: 150, SEQ ID NO: 151, SEQ ID NO: 152, SEQ ID NO: 158, SEQ ID NO: 159, SEQ ID NO: 162, SEQ ID NO: 163, SEQ ID NO: 166, SEQ ID NO: 167, SEQ ID NO: 169, SEQ ID NO: 170, SEQ ID NO: 172, SEQ ID NO: 173, SEQ ID NO: 174, SEQ ID NO: 176, SEQ ID NO: 178, SEQ ID NO: 179, SEQ ID NO: 180, SEQ ID NO: 182, SEQ ID NO: 183, SEQ ID NO: 184, SEQ ID NO: 187, SEQ ID NO: 188, SEQ ID NO: 189, SEQ ID NO: 190, SEQ ID NO: 191, SEQ ID NO: 195, SEQ ID NO: 198, SEQ ID NO: 200, SEQ ID NO: 201, SEQ ID NO: 204, SEQ ID NO: 206, SEQ ID NO: 208, SEQ ID NO: 213, SEQ ID NO: 214, and SEQ ID NO: 216. In certain embodiments, the second polypeptide has a sequence selected from SEQ ID NO: 11, SEQ ID NO: 150, SEQ ID NO: 151, SEQ ID NO: 152, SEQ ID NO: 159, SEQ ID NO: 162, SEQ ID NO: 166, SEQ ID NO: 169, SEQ ID NO: 170, SEQ ID NO: 178, SEQ ID NO: 180, SEQ ID NO: 182, SEQ ID NO: 183, SEQ ID NO: 184, SEQ ID NO: 187, SEQ ID NO: 188, SEQ ID NO: 189, SEQ ID NO: 190, SEQ ID NO: 191, SEQ ID NO: 195, SEQ ID NO: 198, SEQ ID NO: 200, SEQ ID NO: 206, SEQ ID NO: 208, and SEQ ID NO: 214. In certain embodiments, the second polypeptide has the sequence of SEQ ID NO: 11. In certain embodiments, the second polypeptide has the sequence of SEQ ID NO: 150. In certain embodiments, the second polypeptide has the sequence of SEQ ID NO: 151. In certain embodiments, the second polypeptide has the sequence of SEQ ID NO: 152. In certain embodiments, the second polypeptide has the sequence of SEQ ID NO: 158. In certain embodiments, the second polypeptide has the sequence of SEQ ID NO: 159. In certain embodiments, the second polypeptide has the sequence of SEQ ID NO: 160. In certain embodiments, the second polypeptide has the sequence of SEQ ID NO: 161. In certain embodiments, the second polypeptide has the sequence of SEQ ID NO: 162. In certain embodiments, the second polypeptide has the sequence of SEQ ID NO: 163. In certain embodiments, the second polypeptide has the sequence of SEQ ID NO: 164. In certain embodiments, the second polypeptide has the sequence of SEQ ID NO: 165. In certain embodiments, the second polypeptide has the sequence of SEQ ID NO: 166. In certain embodiments, the second polypeptide has the sequence of SEQ ID NO: 167. In certain embodiments, the second polypeptide has the sequence of SEQ ID NO: 168. In certain embodiments, the second polypeptide has the sequence of SEQ ID NO: 169. In certain embodiments, the second polypeptide has the sequence of SEQ ID NO: 170. In certain embodiments, the second polypeptide has the sequence of SEQ ID NO: 171. In certain embodiments, the second polypeptide has the sequence of SEQ ID NO: 172. In certain embodiments, the second polypeptide has the sequence of SEQ ID NO: 173. In certain embodiments, the second polypeptide has the sequence of SEQ ID NO: 174. In certain embodiments, the second polypeptide has the sequence of SEQ ID NO: 175. In certain embodiments, the second polypeptide has the sequence of SEQ ID NO: 176. In certain embodiments, the second polypeptide has the sequence of SEQ ID NO: 177. In certain embodiments, the second polypeptide has the sequence of SEQ ID NO: 178. In certain embodiments, the second polypeptide has the sequence of SEQ ID NO: 179. In certain embodiments, the second polypeptide has the sequence of SEQ ID NO: 180. In certain embodiments, the second polypeptide has the sequence of SEQ ID NO: 181. In certain embodiments, the second polypeptide has the sequence of SEQ ID NO: 182. In certain embodiments, the second polypeptide has the sequence of SEQ ID NO: 183. In certain embodiments, the second polypeptide has the sequence of SEQ ID NO: 184. In certain embodiments, the second polypeptide has the sequence of SEQ ID NO: 185. In certain embodiments, the second polypeptide has the sequence of SEQ ID NO: 186. In certain embodiments, the second polypeptide has the sequence of SEQ ID NO: 187. In certain embodiments, the second polypeptide has the sequence of SEQ ID NO: 188. In certain embodiments, the second polypeptide has the sequence of SEQ ID NO: 189. In certain embodiments, the second polypeptide has the sequence of SEQ ID NO: 190. In certain embodiments, the second polypeptide has the sequence of SEQ ID NO: 191. In certain embodiments, the second polypeptide has the sequence of SEQ ID NO: 192. In certain embodiments, the second polypeptide has the sequence of SEQ ID NO: 193. In certain embodiments, the second polypeptide has the sequence of SEQ ID NO: 194. In certain embodiments, the second polypeptide has the sequence of SEQ ID NO: 195. In certain embodiments, the second polypeptide has the sequence of SEQ ID NO: 196. In certain embodiments, the second polypeptide has the sequence of SEQ ID NO: 197. In certain embodiments, the second polypeptide has the sequence of SEQ ID NO: 198. In certain embodiments, the second polypeptide has the sequence of SEQ ID NO: 199. In certain embodiments, the second polypeptide has the sequence of SEQ ID NO: 200. In certain embodiments, the second polypeptide has the sequence of SEQ ID NO: 201. In certain embodiments, the second polypeptide has the sequence of SEQ ID NO: 202. In certain embodiments, the second polypeptide has the sequence of SEQ ID NO: 203. In certain embodiments, the second polypeptide has the sequence of SEQ ID NO: 204. In certain embodiments, the second polypeptide has the sequence of SEQ ID NO: 205. In certain embodiments, the second polypeptide has the sequence of SEQ ID NO: 206. In certain embodiments, the second polypeptide has the sequence of SEQ ID NO: 207. In certain embodiments, the second polypeptide has the sequence of SEQ ID NO: 208. In certain embodiments, the second polypeptide has the sequence of SEQ ID NO: 209. In certain embodiments, the second polypeptide has the sequence of SEQ ID NO: 210. In certain embodiments, the second polypeptide has the sequence of SEQ ID NO: 211. In certain embodiments, the second polypeptide has the sequence of SEQ ID NO: 212. In certain embodiments, the second polypeptide has the sequence of SEQ ID NO: 213. In certain embodiments, the second polypeptide has the sequence of SEQ ID NO: 214. In certain embodiments, the second polypeptide has the sequence of SEQ ID NO: 215. In certain embodiments, the second polypeptide has the sequence of SEQ ID NO: 216.

In certain embodiments, the third polypeptide has a sequence selected from SEQ ID NOs: 12 and 55-149. In certain embodiments, the third polypeptide has a sequence selected from SEQ ID NOs: 55-149. In certain embodiments, the third polypeptide has the sequence of SEQ ID NO: 12. In certain embodiments, the third polypeptide has the sequence of SEQ ID NO: 55. In certain embodiments, the third polypeptide has the sequence of SEQ ID NO: 56. In certain embodiments, the third polypeptide has the sequence of SEQ ID NO: 57. In certain embodiments, the third polypeptide has the sequence of SEQ ID NO: 58. In certain embodiments, the third polypeptide has the sequence of SEQ ID NO: 59. In certain embodiments, the third polypeptide has the sequence of SEQ ID NO: 60. In certain embodiments, the third polypeptide has the sequence of SEQ ID NO: 61. In certain embodiments, the third polypeptide has the sequence of SEQ ID NO: 62. In certain embodiments, the third polypeptide has the sequence of SEQ ID NO: 63. In certain embodiments, the third polypeptide has the sequence of SEQ ID NO: 64. In certain embodiments, the third polypeptide has the sequence of SEQ ID NO: 65. In certain embodiments, the third polypeptide has the sequence of SEQ ID NO: 66. In certain embodiments, the third polypeptide has the sequence of SEQ ID NO: 67. In certain embodiments, the third polypeptide has the sequence of SEQ ID NO: 68. In certain embodiments, the third polypeptide has the sequence of SEQ ID NO: 69. In certain embodiments, the third polypeptide has the sequence of SEQ ID NO: 70. In certain embodiments, the third polypeptide has the sequence of SEQ ID NO: 71. In certain embodiments, the third polypeptide has the sequence of SEQ ID NO: 72. In certain embodiments, the third polypeptide has the sequence of SEQ ID NO: 73. In certain embodiments, the third polypeptide has the sequence of SEQ ID NO: 74. In certain embodiments, the third polypeptide has the sequence of SEQ ID NO: 75. In certain embodiments, the third polypeptide has the sequence of SEQ ID NO: 76. In certain embodiments, the third polypeptide has the sequence of SEQ ID NO: 77. In certain embodiments, the third polypeptide has the sequence of SEQ ID NO: 78. In certain embodiments, the third polypeptide has the sequence of SEQ ID NO: 79. In certain embodiments, the third polypeptide has the sequence of SEQ ID NO: 80. In certain embodiments, the third polypeptide has the sequence of SEQ ID NO: 81. In certain embodiments, the third polypeptide has the sequence of SEQ ID NO: 82. In certain embodiments, the third polypeptide has the sequence of SEQ ID NO: 83. In certain embodiments, the third polypeptide has the sequence of SEQ ID NO: 84. In certain embodiments, the third polypeptide has the sequence of SEQ ID NO: 85. In certain embodiments, the third polypeptide has the sequence of SEQ ID NO: 86. In certain embodiments, the third polypeptide has the sequence of SEQ ID NO: 87. In certain embodiments, the third polypeptide has the sequence of SEQ ID NO: 88. In certain embodiments, the third polypeptide has the sequence of SEQ ID NO: 89. In certain embodiments, the third polypeptide has the sequence of SEQ ID NO: 90. In certain embodiments, the third polypeptide has the sequence of SEQ ID NO: 91. In certain embodiments, the third polypeptide has the sequence of SEQ ID NO: 92. In certain embodiments, the third polypeptide has the sequence of SEQ ID NO: 93. In certain embodiments, the third polypeptide has the sequence of SEQ ID NO: 94. In certain embodiments, the third polypeptide has the sequence of SEQ ID NO: 95. In certain embodiments, the third polypeptide has the sequence of SEQ ID NO: 96. In certain embodiments, the third polypeptide has the sequence of SEQ ID NO: 97. In certain embodiments, the third polypeptide has the sequence of SEQ ID NO: 98. In certain embodiments, the third polypeptide has the sequence of SEQ ID NO: 99. In certain embodiments, the third polypeptide has the sequence of SEQ ID NO: 100. In certain embodiments, the third polypeptide has the sequence of SEQ ID NO: 101. In certain embodiments, the third polypeptide has the sequence of SEQ ID NO: 102. In certain embodiments, the third polypeptide has the sequence of SEQ ID NO: 103. In certain embodiments, the third polypeptide has the sequence of SEQ ID NO: 104. In certain embodiments, the third polypeptide has the sequence of SEQ ID NO: 105. In certain embodiments, the third polypeptide has the sequence of SEQ ID NO: 106. In certain embodiments, the third polypeptide has the sequence of SEQ ID NO: 107. In certain embodiments, the third polypeptide has the sequence of SEQ ID NO: 108. In certain embodiments, the third polypeptide has the sequence of SEQ ID NO: 109. In certain embodiments, the third polypeptide has the sequence of SEQ ID NO: 110. In certain embodiments, the third polypeptide has the sequence of SEQ ID NO: 111. In certain embodiments, the third polypeptide has the sequence of SEQ ID NO: 112. In certain embodiments, the third polypeptide has the sequence of SEQ ID NO: 113. In certain embodiments, the third polypeptide has the sequence of SEQ ID NO: 114. In certain embodiments, the third polypeptide has the sequence of SEQ ID NO: 115. In certain embodiments, the third polypeptide has the sequence of SEQ ID NO: 116. In certain embodiments, the third polypeptide has the sequence of SEQ ID NO: 117. In certain embodiments, the third polypeptide has the sequence of SEQ ID NO: 118. In certain embodiments, the third polypeptide has the sequence of SEQ ID NO: 119. In certain embodiments, the third polypeptide has the sequence of SEQ ID NO: 120. In certain embodiments, the third polypeptide has the sequence of SEQ ID NO: 121. In certain embodiments, the third polypeptide has the sequence of SEQ ID NO: 122. In certain embodiments, the third polypeptide has the sequence of SEQ ID NO: 123. In certain embodiments, the third polypeptide has the sequence of SEQ ID NO: 124. In certain embodiments, the third polypeptide has the sequence of SEQ ID NO: 125. In certain embodiments, the third polypeptide has the sequence of SEQ ID NO: 126. In certain embodiments, the third polypeptide has the sequence of SEQ ID NO: 127. In certain embodiments, the third polypeptide has the sequence of SEQ ID NO: 128. In certain embodiments, the third polypeptide has the sequence of SEQ ID NO: 129. In certain embodiments, the third polypeptide has the sequence of SEQ ID NO: 130. In certain embodiments, the third polypeptide has the sequence of SEQ ID NO: 131. In certain embodiments, the third polypeptide has the sequence of SEQ ID NO: 132. In certain embodiments, the third polypeptide has the sequence of SEQ ID NO: 133. In certain embodiments, the third polypeptide has the sequence of SEQ ID NO: 134. In certain embodiments, the third polypeptide has the sequence of SEQ ID NO: 135. In certain embodiments, the third polypeptide has the sequence of SEQ ID NO: 136. In certain embodiments, the third polypeptide has the sequence of SEQ ID NO: 137. In certain embodiments, the third polypeptide has the sequence of SEQ ID NO: 138. In certain embodiments, the third polypeptide has the sequence of SEQ ID NO: 139. In certain embodiments, the third polypeptide has the sequence of SEQ ID NO: 140. In certain embodiments, the third polypeptide has the sequence of SEQ ID NO: 141. In certain embodiments, the third polypeptide has the sequence of SEQ ID NO: 142. In certain embodiments, the third polypeptide has the sequence of SEQ ID NO: 143. In certain embodiments, the third polypeptide has the sequence of SEQ ID NO: 144. In certain embodiments, the third polypeptide has the sequence of SEQ ID NO: 145. In certain embodiments, the third polypeptide has the sequence of SEQ ID NO: 146. In certain embodiments, the third polypeptide has the sequence of SEQ ID NO: 147. In certain embodiments, the third polypeptide has the sequence of SEQ ID NO: 148. In certain embodiments, the third polypeptide has the sequence of SEQ ID NO: 149.

In certain embodiments, the fourth polypeptide has a sequence selected from SEQ ID NOs: 14-54. In certain embodiments, the fourth polypeptide has the sequence of SEQ ID NO: 14. In certain embodiments, the fourth polypeptide has the sequence of SEQ ID NO: 15. In certain embodiments, the fourth polypeptide has the sequence of SEQ ID NO: 16. In certain embodiments, the fourth polypeptide has the sequence of SEQ ID NO: 17. In certain embodiments, the fourth polypeptide has the sequence of SEQ ID NO: 18. In certain embodiments, the fourth polypeptide has the sequence of SEQ ID NO: 19. In certain embodiments, the fourth polypeptide has the sequence of SEQ ID NO: 20. In certain embodiments, the fourth polypeptide has the sequence of SEQ ID NO: 21. In certain embodiments, the fourth polypeptide has the sequence of SEQ ID NO: 22. In certain embodiments, the fourth polypeptide has the sequence of SEQ ID NO: 23. In certain embodiments, the fourth polypeptide has the sequence of SEQ ID NO: 24. In certain embodiments, the fourth polypeptide has the sequence of SEQ ID NO: 25. In certain embodiments, the fourth polypeptide has the sequence of SEQ ID NO: 26. In certain embodiments, the fourth polypeptide has the sequence of SEQ ID NO: 27. In certain embodiments, the fourth polypeptide has the sequence of SEQ ID NO: 28. In certain embodiments, the fourth polypeptide has the sequence of SEQ ID NO: 29. In certain embodiments, the fourth polypeptide has the sequence of SEQ ID NO: 30. In certain embodiments, the fourth polypeptide has the sequence of SEQ ID NO: 31. In certain embodiments, the fourth polypeptide has the sequence of SEQ ID NO: 32. In certain embodiments, the fourth polypeptide has the sequence of SEQ ID NO: 33. In certain embodiments, the fourth polypeptide has the sequence of SEQ ID NO: 34. In certain embodiments, the fourth polypeptide has the sequence of SEQ ID NO: 35. In certain embodiments, the fourth polypeptide has the sequence of SEQ ID NO: 36. In certain embodiments, the fourth polypeptide has the sequence of SEQ ID NO: 37. In certain embodiments, the fourth polypeptide has the sequence of SEQ ID NO: 38. In certain embodiments, the fourth polypeptide has the sequence of SEQ ID NO: 39. In certain embodiments, the fourth polypeptide has the sequence of SEQ ID NO: 40. In certain embodiments, the fourth polypeptide has the sequence of SEQ ID NO: 41. In certain embodiments, the fourth polypeptide has the sequence of SEQ ID NO: 42. In certain embodiments, the fourth polypeptide has the sequence of SEQ ID NO: 43. In certain embodiments, the fourth polypeptide has the sequence of SEQ ID NO: 44. In certain embodiments, the fourth polypeptide has the sequence of SEQ ID NO: 45. In certain embodiments, the fourth polypeptide has the sequence of SEQ ID NO: 46. In certain embodiments, the fourth polypeptide has the sequence of SEQ ID NO: 47. In certain embodiments, the fourth polypeptide has the sequence of SEQ ID NO: 48. In certain embodiments, the fourth polypeptide has the sequence of SEQ ID NO: 49. In certain embodiments, the fourth polypeptide has the sequence of SEQ ID NO: 50. In certain embodiments, the fourth polypeptide has the sequence of SEQ ID NO: 51. In certain embodiments, the fourth polypeptide has the sequence of SEQ ID NO: 52. In certain embodiments, the fourth polypeptide has the sequence of SEQ ID NO: 53. In certain embodiments, the fourth polypeptide has the sequence of SEQ ID NO: 54.

In certain embodiments, the first polypeptide lacks lycopene cyclase activity. In certain embodiments, the first polypeptide has a sequence having at least 80, 85, 90, 95, 99, or 100% identity to SEQ ID NO: 241 or 242. In certain embodiments, the first polypeptide has the sequence of SEQ ID NO: 241 or 242.

In certain embodiments, the recombinant host cell further comprises a heterologous nucleic acid encoding a lycopene cyclase.

In certain embodiments, the lycopene cyclase has a sequence having at least 80, 85, 90, 95, 99, or 100% identity to any one of SEQ ID NOs: 243-288. In certain embodiments, the lycopene cyclase has a sequence having at least 80, 85, 90, 95, 99, or 100% identity to any one of SEQ ID NOs: 243-273. In certain embodiments, the lycopene cyclase has a sequence having at least 80, 85, 90, 95, 99, or 100% identity to SEQ ID NO: 243. In certain embodiments, the lycopene cyclase has a sequence having at least 80, 85, 90, 95, 99, or 100% identity to SEQ ID NO: 244. In certain embodiments, the lycopene cyclase has a sequence having at least 80, 85, 90, 95, 99, or 100% identity to SEQ ID NO: 245. In certain embodiments, the lycopene cyclase has a sequence having at least 80, 85, 90, 95, 99, or 100% identity to SEQ ID NO: 246. In certain embodiments, the lycopene cyclase has a sequence having at least 80, 85, 90, 95, 99, or 100% identity to SEQ ID NO: 247. In certain embodiments, the lycopene cyclase has a sequence having at least 80, 85, 90, 95, 99, or 100% identity to SEQ ID NO: 248. In certain embodiments, the lycopene cyclase has a sequence having at least 80, 85, 90, 95, 99, or 100% identity to SEQ ID NO: 249. In certain embodiments, the lycopene cyclase has a sequence having at least 80, 85, 90, 95, 99, or 100% identity to SEQ ID NO: 250. In certain embodiments, the lycopene cyclase has a sequence having at least 80, 85, 90, 95, 99, or 100% identity to SEQ ID NO: 251. In certain embodiments, the lycopene cyclase has a sequence having at least 80, 85, 90, 95, 99, or 100% identity to SEQ ID NO: 252. In certain embodiments, the lycopene cyclase has a sequence having at least 80, 85, 90, 95, 99, or 100% identity to SEQ ID NO: 253. In certain embodiments, the lycopene cyclase has a sequence having at least 80, 85, 90, 95, 99, or 100% identity to SEQ ID NO: 254. In certain embodiments, the lycopene cyclase has a sequence having at least 80, 85, 90, 95, 99, or 100% identity to SEQ ID NO: 255. In certain embodiments, the lycopene cyclase has a sequence having at least 80, 85, 90, 95, 99, or 100% identity to SEQ ID NO: 256. In certain embodiments, the lycopene cyclase has a sequence having at least 80, 85, 90, 95, 99, or 100% identity to SEQ ID NO: 257. In certain embodiments, the lycopene cyclase has a sequence having at least 80, 85, 90, 95, 99, or 100% identity to SEQ ID NO: 258. In certain embodiments, the lycopene cyclase has a sequence having at least 80, 85, 90, 95, 99, or 100% identity to SEQ ID NO: 259. In certain embodiments, the lycopene cyclase has a sequence having at least 80, 85, 90, 95, 99, or 100% identity to SEQ ID NO: 260. In certain embodiments, the lycopene cyclase has a sequence having at least 80, 85, 90, 95, 99, or 100% identity to SEQ ID NO: 261. In certain embodiments, the lycopene cyclase has a sequence having at least 80, 85, 90, 95, 99, or 100% identity to SEQ ID NO: 262. In certain embodiments, the lycopene cyclase has a sequence having at least 80, 85, 90, 95, 99, or 100% identity to SEQ ID NO: 263. In certain embodiments, the lycopene cyclase has a sequence having at least 80, 85, 90, 95, 99, or 100% identity to SEQ ID NO: 264. In certain embodiments, the lycopene cyclase has a sequence having at least 80, 85, 90, 95, 99, or 100% identity to SEQ ID NO: 265. In certain embodiments, the lycopene cyclase has a sequence having at least 80, 85, 90, 95, 99, or 100% identity to SEQ ID NO: 266. In certain embodiments, the lycopene cyclase has a sequence having at least 80, 85, 90, 95, 99, or 100% identity to SEQ ID NO: 267. In certain embodiments, the lycopene cyclase has a sequence having at least 80, 85, 90, 95, 99, or 100% identity to SEQ ID NO: 268. In certain embodiments, the lycopene cyclase has a sequence having at least 80, 85, 90, 95, 99, or 100% identity to SEQ ID NO: 269. In certain embodiments, the lycopene cyclase has a sequence having at least 80, 85, 90, 95, 99, or 100% identity to SEQ ID NO: 270. In certain embodiments, the lycopene cyclase has a sequence having at least 80, 85, 90, 95, 99, or 100% identity to SEQ ID NO: 271. In certain embodiments, the lycopene cyclase has a sequence having at least 80, 85, 90, 95, 99, or 100% identity to SEQ ID NO: 272. In certain embodiments, the lycopene cyclase has a sequence having at least 80, 85, 90, 95, 99, or 100% identity to SEQ ID NO: 273. In certain embodiments, the lycopene cyclase has a sequence having at least 80, 85, 90, 95, 99, or 100% identity to SEQ ID NO: 274. In certain embodiments, the lycopene cyclase has a sequence having at least 80, 85, 90, 95, 99, or 100% identity to SEQ ID NO: 275. In certain embodiments, the lycopene cyclase has a sequence having at least 80, 85, 90, 95, 99, or 100% identity to SEQ ID NO: 276. In certain embodiments, the lycopene cyclase has a sequence having at least 80, 85, 90, 95, 99, or 100% identity to SEQ ID NO: 277. In certain embodiments, the lycopene cyclase has a sequence having at least 80, 85, 90, 95, 99, or 100% identity to SEQ ID NO: 278. In certain embodiments, the lycopene cyclase has a sequence having at least 80, 85, 90, 95, 99, or 100% identity to SEQ ID NO: 279. In certain embodiments, the lycopene cyclase has a sequence having at least 80, 85, 90, 95, 99, or 100% identity to SEQ ID NO: 280. In certain embodiments, the lycopene cyclase has a sequence having at least 80, 85, 90, 95, 99, or 100% identity to SEQ ID NO: 281. In certain embodiments, the lycopene cyclase has a sequence having at least 80, 85, 90, 95, 99, or 100% identity to SEQ ID NO: 282. In certain embodiments, the lycopene cyclase has a sequence having at least 80, 85, 90, 95, 99, or 100% identity to SEQ ID NO: 283. In certain embodiments, the lycopene cyclase has a sequence having at least 80, 85, 90, 95, 99, or 100% identity to SEQ ID NO: 284. In certain embodiments, the lycopene cyclase has a sequence having at least 80, 85, 90, 95, 99, or 100% identity to SEQ ID NO: 285. In certain embodiments, the lycopene cyclase has a sequence having at least 80, 85, 90, 95, 99, or 100% identity to SEQ ID NO: 286. In certain embodiments, the lycopene cyclase has a sequence having at least 80, 85, 90, 95, 99, or 100% identity to SEQ ID NO: 287. In certain embodiments, the lycopene cyclase has a sequence having at least 80, 85, 90, 95, 99, or 100% identity to SEQ ID NO: 288.

In certain embodiments, the lycopene cyclase has the sequence of any one of SEQ ID NOs: 243-288. In certain embodiments, the lycopene cyclase has the sequence of any one of SEQ ID NOs: 243-273. In certain embodiments, the lycopene cyclase has the sequence of SEQ ID NO: 243. In certain embodiments, the lycopene cyclase has the sequence of SEQ ID NO: 244. In certain embodiments, the lycopene cyclase has the sequence of SEQ ID NO: 245. In certain embodiments, the lycopene cyclase has the sequence of SEQ ID NO: 246. In certain embodiments, the lycopene cyclase has the sequence of SEQ ID NO: 247. In certain embodiments, the lycopene cyclase has the sequence of SEQ ID NO: 248. In certain embodiments, the lycopene cyclase has the sequence of SEQ ID NO: 249. In certain embodiments, the lycopene cyclase has the sequence of SEQ ID NO: 250. In certain embodiments, the lycopene cyclase has the sequence of SEQ ID NO: 251. In certain embodiments, the lycopene cyclase has the sequence of SEQ ID NO: 252. In certain embodiments, the lycopene cyclase has the sequence of SEQ ID NO: 253. In certain embodiments, the lycopene cyclase has the sequence of SEQ ID NO: 254. In certain embodiments, the lycopene cyclase has the sequence of SEQ ID NO: 255. In certain embodiments, the lycopene cyclase has the sequence of SEQ ID NO: 256. In certain embodiments, the lycopene cyclase has the sequence of SEQ ID NO: 257. In certain embodiments, the lycopene cyclase has the sequence of SEQ ID NO: 258. In certain embodiments, the lycopene cyclase has the sequence of SEQ ID NO: 259. In certain embodiments, the lycopene cyclase has the sequence of SEQ ID NO: 260. In certain embodiments, the lycopene cyclase has the sequence of SEQ ID NO: 261. In certain embodiments, the lycopene cyclase has the sequence of SEQ ID NO: 262. In certain embodiments, the lycopene cyclase has the sequence of SEQ ID NO: 263. In certain embodiments, the lycopene cyclase has the sequence of SEQ ID NO: 264. In certain embodiments, the lycopene cyclase has the sequence of SEQ ID NO: 265. In certain embodiments, the lycopene cyclase has the sequence of SEQ ID NO: 266. In certain embodiments, the lycopene cyclase has the sequence of SEQ ID NO: 267. In certain embodiments, the lycopene cyclase has the sequence of SEQ ID NO: 268. In certain embodiments, the lycopene cyclase has the sequence of SEQ ID NO: 269. In certain embodiments, the lycopene cyclase has the sequence of SEQ ID NO: 270. In certain embodiments, the lycopene cyclase has the sequence of SEQ ID NO: 271. In certain embodiments, the lycopene cyclase has the sequence of SEQ ID NO: 272. In certain embodiments, the lycopene cyclase has the sequence of SEQ ID NO: 273. In certain embodiments, the lycopene cyclase has the sequence of SEQ ID NO: 274. In certain embodiments, the lycopene cyclase has the sequence of SEQ ID NO: 275. In certain embodiments, the lycopene cyclase has the sequence of SEQ ID NO: 276. In certain embodiments, the lycopene cyclase has the sequence of SEQ ID NO: 277. In certain embodiments, the lycopene cyclase has the sequence of SEQ ID NO: 278. In certain embodiments, the lycopene cyclase has the sequence of SEQ ID NO: 279. In certain embodiments, the lycopene cyclase has the sequence of SEQ ID NO: 280. In certain embodiments, the lycopene cyclase has the sequence of SEQ ID NO: 281. In certain embodiments, the lycopene cyclase has the sequence of SEQ ID NO: 282. In certain embodiments, the lycopene cyclase has the sequence of SEQ ID NO: 283. In certain embodiments, the lycopene cyclase has the sequence of SEQ ID NO: 284. In certain embodiments, the lycopene cyclase has the sequence of SEQ ID NO: 285. In certain embodiments, the lycopene cyclase has the sequence of SEQ ID NO: 286. In certain embodiments, the lycopene cyclase has the sequence of SEQ ID NO: 287. In certain embodiments, the lycopene cyclase has the sequence of SEQ ID NO: 288.

In additional embodiments, the recombinant host cell further comprises one or more heterologous nucleic acids that encode one or more polypeptides having a sequence having at least 80, 85, 90, 95, 99 or 100% identity to a sequence selected from SEQ ID NO: 3, SEQ ID NO: 1, SEQ ID NO: 7, SEQ ID NO: 157, SEQ ID NO: 2, SEQ ID NO: 8, SEQ ID NO: 5, SEQ ID NO: 6, and SEQ ID NO: 4. In some embodiments, the one or more polypeptides have a sequence selected from SEQ ID NO: 3, SEQ ID NO: 1, SEQ ID NO: 7, SEQ ID NO: 157, SEQ ID NO: 2, SEQ ID NO: 8, SEQ ID NO: 5, SEQ ID NO: 6, and SEQ ID NO: 4. In certain embodiments, the recombinant host cell further comprises one or more heterologous nucleic acids that encode polypeptides having a sequence having at least 80, 85, 90, 95, 99, or 100% identity to SEQ ID NO: 1, SEQ ID NO: 2, SEQ ID NO: 3 or 4, SEQ ID NO: 5 or 157, SEQ ID NO: 6, SEQ ID NO: 7, and SEQ ID NO: 8. In certain embodiments, the recombinant host cell further comprises one or more heterologous nucleic acids that encode polypeptides having the sequences SEQ ID NO: 1, SEQ ID NO: 2, SEQ ID NO: 3 or 4, SEQ ID NO: 5 or 157, SEQ ID NO: 6, SEQ ID NO: 7, and SEQ ID NO: 8.

In further embodiments, the recombinant host cell further contains a heterologous nucleic acid that encodes a geranylgeranyl diphosphate synthase having a sequence having at least 80, 85, 90, 95, 99, or 100% identity to a sequence selected from SEQ ID NOs: 9 and 289-327. In further embodiments, the recombinant host cell further contains a heterologous nucleic acid that encodes a geranylgeranyl diphosphate synthase having a sequence having at least 80, 85, 90, 95, 99, or 100% identity to a sequence selected from SEQ ID NOs: 9 and 289-310. In certain embodiments, the geranylgeranyl diphosphate synthase has a sequence having at least 80, 85, 90, 95, 99, or 100% identity to SEQ ID NO: 9. In certain embodiments, the geranylgeranyl diphosphate synthase has a sequence having at least 80, 85, 90, 95, 99, or 100% identity to SEQ ID NO: 289. In certain embodiments, the geranylgeranyl diphosphate synthase has a sequence having at least 80, 85, 90, 95, 99, or 100% identity to SEQ ID NO: 290. In certain embodiments, the geranylgeranyl diphosphate synthase has a sequence having at least 80, 85, 90, 95, 99, or 100% identity to SEQ ID NO: 291. In certain embodiments, the geranylgeranyl diphosphate synthase has a sequence having at least 80, 85, 90, 95, 99, or 100% identity to SEQ ID NO: 292. In certain embodiments, the geranylgeranyl diphosphate synthase has a sequence having at least 80, 85, 90, 95, 99, or 100% identity to SEQ ID NO: 293. In certain embodiments, the geranylgeranyl diphosphate synthase has a sequence having at least 80, 85, 90, 95, 99, or 100% identity to SEQ ID NO: 294. In certain embodiments, the geranylgeranyl diphosphate synthase has a sequence having at least 80, 85, 90, 95, 99, or 100% identity to SEQ ID NO: 295. In certain embodiments, the geranylgeranyl diphosphate synthase has a sequence having at least 80, 85, 90, 95, 99, or 100% identity to SEQ ID NO: 296. In certain embodiments, the geranylgeranyl diphosphate synthase has a sequence having at least 80, 85, 90, 95, 99, or 100% identity to SEQ ID NO: 297. In certain embodiments, the geranylgeranyl diphosphate synthase has a sequence having at least 80, 85, 90, 95, 99, or 100% identity to SEQ ID NO: 298. In certain embodiments, the geranylgeranyl diphosphate synthase has a sequence having at least 80, 85, 90, 95, 99, or 100% identity to SEQ ID NO: 299. In certain embodiments, the geranylgeranyl diphosphate synthase has a sequence having at least 80, 85, 90, 95, 99, or 100% identity to SEQ ID NO: 300. In certain embodiments, the geranylgeranyl diphosphate synthase has a sequence having at least 80, 85, 90, 95, 99, or 100% identity to SEQ ID NO: 301. In certain embodiments, the geranylgeranyl diphosphate synthase has a sequence having at least 80, 85, 90, 95, 99, or 100% identity to SEQ ID NO: 302. In certain embodiments, the geranylgeranyl diphosphate synthase has a sequence having at least 80, 85, 90, 95, 99, or 100% identity to SEQ ID NO: 303. In certain embodiments, the geranylgeranyl diphosphate synthase has a sequence having at least 80, 85, 90, 95, 99, or 100% identity to SEQ ID NO: 304. In certain embodiments, the geranylgeranyl diphosphate synthase has a sequence having at least 80, 85, 90, 95, 99, or 100% identity to SEQ ID NO: 305. In certain embodiments, the geranylgeranyl diphosphate synthase has a sequence having at least 80, 85, 90, 95, 99, or 100% identity to SEQ ID NO: 306. In certain embodiments, the geranylgeranyl diphosphate synthase has a sequence having at least 80, 85, 90, 95, 99, or 100% identity to SEQ ID NO: 307. In certain embodiments, the geranylgeranyl diphosphate synthase has a sequence having at least 80, 85, 90, 95, 99, or 100% identity to SEQ ID NO: 308. In certain embodiments, the geranylgeranyl diphosphate synthase has a sequence having at least 80, 85, 90, 95, 99, or 100% identity to SEQ ID NO: 309. In certain embodiments, the geranylgeranyl diphosphate synthase has a sequence having at least 80, 85, 90, 95, 99, or 100% identity to SEQ ID NO: 310. In certain embodiments, the geranylgeranyl diphosphate synthase has a sequence having at least 80, 85, 90, 95, 99, or 100% identity to SEQ ID NO: 311. In certain embodiments, the geranylgeranyl diphosphate synthase has a sequence having at least 80, 85, 90, 95, 99, or 100% identity to SEQ ID NO: 312. In certain embodiments, the geranylgeranyl diphosphate synthase has a sequence having at least 80, 85, 90, 95, 99, or 100% identity to SEQ ID NO: 313. In certain embodiments, the geranylgeranyl diphosphate synthase has a sequence having at least 80, 85, 90, 95, 99, or 100% identity to SEQ ID NO: 314. In certain embodiments, the geranylgeranyl diphosphate synthase has a sequence having at least 80, 85, 90, 95, 99, or 100% identity to SEQ ID NO: 315. In certain embodiments, the geranylgeranyl diphosphate synthase has a sequence having at least 80, 85, 90, 95, 99, or 100% identity to SEQ ID NO: 316. In certain embodiments, the geranylgeranyl diphosphate synthase has a sequence having at least 80, 85, 90, 95, 99, or 100% identity to SEQ ID NO: 317. In certain embodiments, the geranylgeranyl diphosphate synthase has a sequence having at least 80, 85, 90, 95, 99, or 100% identity to SEQ ID NO: 318. In certain embodiments, the geranylgeranyl diphosphate synthase has a sequence having at least 80, 85, 90, 95, 99, or 100% identity to SEQ ID NO: 319. In certain embodiments, the geranylgeranyl diphosphate synthase has a sequence having at least 80, 85, 90, 95, 99, or 100% identity to SEQ ID NO: 320. In certain embodiments, the geranylgeranyl diphosphate synthase has a sequence having at least 80, 85, 90, 95, 99, or 100% identity to SEQ ID NO: 321. In certain embodiments, the geranylgeranyl diphosphate synthase has a sequence having at least 80, 85, 90, 95, 99, or 100% identity to SEQ ID NO: 322. In certain embodiments, the geranylgeranyl diphosphate synthase has a sequence having at least 80, 85, 90, 95, 99, or 100% identity to SEQ ID NO: 323. In certain embodiments, the geranylgeranyl diphosphate synthase has a sequence having at least 80, 85, 90, 95, 99, or 100% identity to SEQ ID NO: 324. In certain embodiments, the geranylgeranyl diphosphate synthase has a sequence having at least 80, 85, 90, 95, 99, or 100% identity to SEQ ID NO: 325. In certain embodiments, the geranylgeranyl diphosphate synthase has a sequence having at least 80, 85, 90, 95, 99, or 100% identity to SEQ ID NO: 326. In certain embodiments, the geranylgeranyl diphosphate synthase has a sequence having at least 80, 85, 90, 95, 99, or 100% identity to SEQ ID NO: 327.

In certain embodiments, the geranylgeranyl diphosphate synthase has a sequence selected from SEQ ID NOs: 9 and 289-327. In certain embodiments, the geranylgeranyl diphosphate synthase has a sequence selected from SEQ ID NOs: 9 and 289-310. In certain embodiments, the geranylgeranyl diphosphate synthase has the sequence of SEQ ID NO: 9. In certain embodiments, the geranylgeranyl diphosphate synthase has the sequence of SEQ ID NO: 289. In certain embodiments, the geranylgeranyl diphosphate synthase has the sequence of SEQ ID NO: 290. In certain embodiments, the geranylgeranyl diphosphate synthase has the sequence of SEQ ID NO: 291. In certain embodiments, the geranylgeranyl diphosphate synthase has the sequence of SEQ ID NO: 292. In certain embodiments, the geranylgeranyl diphosphate synthase has the sequence of SEQ ID NO: 293. In certain embodiments, the geranylgeranyl diphosphate synthase has the sequence of SEQ ID NO: 294. In certain embodiments, the geranylgeranyl diphosphate synthase has the sequence of SEQ ID NO: 295. In certain embodiments, the geranylgeranyl diphosphate synthase has the sequence of SEQ ID NO: 296. In certain embodiments, the geranylgeranyl diphosphate synthase has the sequence of SEQ ID NO: 297. In certain embodiments, the geranylgeranyl diphosphate synthase has the sequence of SEQ ID NO: 298. In certain embodiments, the geranylgeranyl diphosphate synthase has the sequence of SEQ ID NO: 299. In certain embodiments, the geranylgeranyl diphosphate synthase has the sequence of SEQ ID NO: 300. In certain embodiments, the geranylgeranyl diphosphate synthase has the sequence of SEQ ID NO: 301. In certain embodiments, the geranylgeranyl diphosphate synthase has the sequence of SEQ ID NO: 302. In certain embodiments, the geranylgeranyl diphosphate synthase has the sequence of SEQ ID NO: 303. In certain embodiments, the geranylgeranyl diphosphate synthase has the sequence of SEQ ID NO: 304. In certain embodiments, the geranylgeranyl diphosphate synthase has the sequence of SEQ ID NO: 305. In certain embodiments, the geranylgeranyl diphosphate synthase has the sequence of SEQ ID NO: 306. In certain embodiments, the geranylgeranyl diphosphate synthase has the sequence of SEQ ID NO: 307. In certain embodiments, the geranylgeranyl diphosphate synthase has the sequence of SEQ ID NO: 308. In certain embodiments, the geranylgeranyl diphosphate synthase has the sequence of SEQ ID NO: 309. In certain embodiments, the geranylgeranyl diphosphate synthase has the sequence of SEQ ID NO: 310. In certain embodiments, the geranylgeranyl diphosphate synthase has the sequence of SEQ ID NO: 311. In certain embodiments, the geranylgeranyl diphosphate synthase has the sequence of SEQ ID NO: 312. In certain embodiments, the geranylgeranyl diphosphate synthase has the sequence of SEQ ID NO: 313. In certain embodiments, the geranylgeranyl diphosphate synthase has the sequence of SEQ ID NO: 314. In certain embodiments, the geranylgeranyl diphosphate synthase has the sequence of SEQ ID NO: 315. In certain embodiments, the geranylgeranyl diphosphate synthase has the sequence of SEQ ID NO: 316. In certain embodiments, the geranylgeranyl diphosphate synthase has the sequence of SEQ ID NO: 317. In certain embodiments, the geranylgeranyl diphosphate synthase has the sequence of SEQ ID NO: 318. In certain embodiments, the geranylgeranyl diphosphate synthase has the sequence of SEQ ID NO: 319. In certain embodiments, the geranylgeranyl diphosphate synthase has the sequence of SEQ ID NO: 320. In certain embodiments, the geranylgeranyl diphosphate synthase has the sequence of SEQ ID NO: 321. In certain embodiments, the geranylgeranyl diphosphate synthase has the sequence of SEQ ID NO: 322. In certain embodiments, the geranylgeranyl diphosphate synthase has the sequence of SEQ ID NO: 323. In certain embodiments, the geranylgeranyl diphosphate synthase has the sequence of SEQ ID NO: 324. In certain embodiments, the geranylgeranyl diphosphate synthase has the sequence of SEQ ID NO: 325. In certain embodiments, the geranylgeranyl diphosphate synthase has the sequence of SEQ ID NO: 326. In certain embodiments, the geranylgeranyl diphosphate synthase has the sequence of SEQ ID NO: 327.

In certain embodiments, the recombinant host cell further comprises a deletion of at least a portion of a native alcohol dehydrogenase gene. In certain embodiments, the native alcohol dehydrogenase gene has a sequence having at least 80, 85, 90, 95, 99, or 100% identity to SEQ ID NO: 13.

In certain embodiments, the recombinant host cell further comprises an ERG9 gene that is downregulated relative to the parent strain.

In further embodiments, the host cell comprises a plant cell, a yeast cell, or a bacterial cell. In other embodiments, the host cell is a yeast cell. In other embodiments, the host cell is a Saccharomyces cerevisiae cell. In certain embodiments, the host cell is a eukaryotic cell. In certain embodiments, the host cell is a prokaryotic cell. In certain embodiments, the host cell is an archaea cell.

In certain embodiments, the host cell is capable of producing farnesene pyrophosphate (FPP). In certain embodiments, the host cell produces farnesene pyrophosphate. In certain embodiments, the host cell is capable of producing geranylgeranyl pyrophosphate (GGPP). In certain embodiment, the host cell produces geranylgeranyl pyrophosphate.

In an additional aspect, the disclosure provides for a method of producing retinol comprising culturing a population of recombinant host cells disclosed herein in a culture medium comprising a carbon source under conditions suitable for making retinol; optionally providing an overlay; and recovering the retinol from the culture medium or the overlay.

In yet another aspect, the disclosure provides for a method of producing lycopene comprising culturing a population of recombinant host cells disclosed herein in a culture medium comprising a carbon source under conditions suitable for making lycopene; optionally providing an overlay; and recovering the lycopene from the culture medium or the overlay.

In a further aspect, the disclosure features a method of producing beta-carotene comprising culturing a population of recombinant host cells disclosed herein in a culture medium comprising a carbon source under conditions suitable for making beta-carotene; optionally providing an overlay; and recovering the beta-carotene from the culture medium or the overlay.

In yet another aspect, the disclosure features a method of producing retinal comprising culturing a population of recombinant host cells disclosed herein in a culture medium comprising a carbon source under conditions suitable for making retinal; optionally providing an overlay; and recovering the retinal from the culture medium or the overlay.

In certain embodiments, the recombinant host cell can comprise the polypeptides. For example, the recombinant host cell can comprise the first polypeptide, second polypeptide, third polypeptide, fourth polypeptide, lycopene cyclase, geranylgeranyl diphosphate synthase, and/or the one or more polypeptides.

### Cell Strains

Host cells of the invention provided herein include archaea, prokaryotic, and eukaryotic cells.

Suitable prokaryotic host cells include, but are not limited to, any of a gram-positive, gran-negative, and gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmus, Serratia, Shigella, Staphylococcus, Streptomyces, Synechococcus, and Zymomonas. Examples of prokaryotic strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. In a particular embodiment, the host cell is an Escherichia coli cell.

Suitable archaea hosts include, but are not limited to, cells belonging to the genera: Aeropyrum, Archaeglobus, Halobacterium, Methanococcus, Methanobacterium, Pyrococcus, Sulfolobus, and Thermoplasma. Examples of archae strains include, but are not limited to: Archaeoglobus fulgidus, Halobacterium sp., Methanococcus jannaschii, Methanobacterium thermoautotrophicum, Thermoplasma acidophilum, Thermoplasma volcanium, Pyrococcus horikoshii, Pyrococcus abyssi, and Aeropyrum pernix.

Suitable eukaryotic hosts include, but are not limited to, fungal cells, algal cells, insect cells, and plant cells. In some embodiments, yeasts useful in the present methods include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malasserzia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastoporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma.

In some embodiments, the host microbe is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorpha (now known as Pichia angusta). In some embodiments, the host microbe is a strain of the genus Candida, such as Candida lipolytica, Candida guilliermondii, Candida krusei, Candida pseudotropicalis, or Candida utils.

In preferred embodiments, the host microbe is Saccharomyces cerevisiae. In some embodiments, the host is a strain of Saccharomyces cerevisiae selected from Baker's yeast, CEN.PK2, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1 BR-1, BR-2, ME-2, VR-2, MA-3, MA-4, CAT-1, CB-1, NR-1, BT-1, and AL-1. In some embodiments, the host microbe is a strain of Saccharomyces cerevisiae selected from PE-2, CAT-1, VR-1, BG-1, CR-1, and SA-1. In a particular embodiment, the strain of Saccharomyces cerevisiae is PE-2. In another particular embodiment, the strain of Saccharomyces cerevisiae is CAT-1. In another particular embodiment, the strain of Saccharomyces cerevisiae is BG-1.

### MEV Pathway FPP

In some embodiments, a genetically modified host cell provided herein comprises one or more heterologous enzymes of the MEV pathway, useful for the formation of FPP and/or GGPP. The one or more enzymes of the MEV pathway may include an enzyme that condenses acetyl-CoA with malonyl-CoA to form acetoacetyl-CoA; an enzyme that condenses two molecules of acetyl-CoA to form acetoacetyl-CoA; an enzyme that condenses acetoacetyl-CoA with acetyl-CoA to form HMG-COA; or an enzyme that converts HMG-COA to mevalonate. In addition, the genetically modified host cells may include a MEV pathway enzyme that phosphorylates mevalonate to mevalonate 5-phosphate; a MEV pathway enzyme that converts mevalonate 5-phosphate to mevalonate 5-pyrophosphate; a MEV pathway enzyme that converts mevalonate 5-pyrophosphate to isopentenyl pyrophosphate; or a MEV pathway enzyme that converts isopentenyl pyrophosphate to dimethylallyl diphosphate. In particular, the one or more enzymes of the MEV pathway are selected from acetyl-CoA thiolase, acetoacetyl-CoA synthetase, HMG-COA synthase, HMG-CoA reductase, mevalonate kinase, phosphomevalonate kinase, mevalonate pyrophosphate decarboxylase, and isopentyl diphosphate: dimethylallyl diphosphate isomerase (IDI or IPP isomerase). The genetically modified host cell of the invention may express one or more of the heterologous enzymes of the MEV from one or more heterologous nucleotide sequences comprising the coding sequence of the one or more MEV pathway enzymes.

In some embodiments, the genetically modified host cell comprises a heterologous nucleic acid encoding an enzyme that can convert isopentenyl pyrophosphate (IPP) into dimethylallyl pyrophosphate (DMAPP). In addition, the host cell may contain a heterologous nucleic acid encoding an enzyme that may condense IPP and/or DMAPP molecules to form a polyprenyl compound. In some embodiments, the genetically modified host cell further contains a heterologous nucleic acid encoding an enzyme that may modify IPP or a polyprenyl to form an isoprenoid compound such as FPP.

### Conversion of Acetyl-CoA to Acetoacetyl-CoA

The genetically modified host cell may contain a heterologous nucleic acid that encodes an enzyme that may condense two molecules of acetyl-coenzyme A to form acetoacetyl-CoA (an acetyl-CoA thiolase). Examples of nucleotide sequences encoding acetyl-CoA thiolase include (accession no. NC_000913 REGION: 2324131.2325315 (Escherichia coli)); (D49362 (Paracoccus denitrificans)); and (L20428 (Saccharomyces cerevisiae)).

Acetyl-CoA thiolase catalyzes the reversible condensation of two molecules of acetyl-CoA to yield acetoacetyl-CoA, but this reaction is thermodynamically unfavorable; acetoacetyl-CoA thiolysis is favored over acetoacetyl-CoA synthesis. Acetoacetyl-CoA synthase (AACS) (also referred to as acetyl-CoA: malonyl-CoA acyltransferase; EC 2.3.1.194) condenses acetyl-CoA with malonyl-CoA to form acetoacetyl-CoA.

### Conversion of Acetoacetyl-CoA to HMG-COA

In some embodiments, the host cell comprises a heterologous nucleotide sequence encoding an enzyme that can condense acetoacetyl-CoA with another molecule of acetyl-CoA to form 3-hydroxy-3-methylglutaryl-CoA (HMG-COA), e.g., a HMG-COA synthase. Examples of nucleotide sequences encoding such an enzyme include: (NC_001145. complement 19061.20536; Saccharomyces cerevisiae), (X96617; Saccharomyces cerevisiae), (X83882; Arabidopsis thaliana), (AB037907; Kitasatospora griseola), (BT007302; Homo sapiens), and (NC_002758, Locus tag SAV2546, GeneID 1122571; Staphylococcus aureus).

### Conversion of HMG-COA to Mevalonate

In some embodiments, the host cell comprises a heterologous nucleotide sequence encoding an enzyme that can convert HMG-CoA into mevalonate, e.g., a HMG-CoA reductase.

Examples of nucleotide sequences encoding an NADPH-using HMG-COA reductase include: (NM_206548; Drosophila melanogaster), (NC_002758, Locus tag SAV2545, GeneID 1122570; Staphylococcus aureus), (AB015627; Streptomyces sp. KO 3988), (AX128213, providing the sequence encoding a truncated HMG-COA reductase; Saccharomyces cerevisiae), and (NC_001145: complement (115734.118898; Saccharomyces cerevisiae).

### Conversion of Mevalonate to Mevalonate-5-Phosphate

The host cell may contain a heterologous nucleotide sequence encoding an enzyme that can convert mevalonate into mevalonate 5-phosphate, e.g., a mevalonate kinase. Illustrative examples of nucleotide sequences encoding such an enzyme include: (L77688; Arabidopsis thaliana) and (X55875; Saccharomyces cerevisiae).

### Conversion of Mevalonate-5-Phosphate to Mevalonate-5-Pyrophosphate

The host cell may contain a heterologous nucleotide sequence encoding an enzyme that can convert mevalonate 5-phosphate into mevalonate 5-pyrophosphate, e.g., a phosphomevalonate kinase. Illustrative examples of nucleotide sequences encoding such an enzyme include: (AF429385; Hevea brasiliensis), (NM_006556; Homo sapiens), and (NC_001145. complement 712315.713670; Saccharomyces cerevisiae).

### Conversion of Mevalonate-5-Pyrophosphate to IPP

The host cell may contain a heterologous nucleotide sequence encoding an enzyme that can convert mevalonate 5-pyrophosphate into isopentenyl diphosphate (IPP), e.g., a mevalonate pyrophosphate decarboxylase. Illustrative examples of nucleotide sequences encoding such an enzyme include: (X97557; Saccharomyces cerevisiae), (AF290095; Enterococcus faecium), and (U49260; Homo sapiens).

### Conversion of IPP to DMAPP

The host cell may contain a heterologous nucleotide sequence encoding an enzyme that can convert IPP generated via the MEV pathway into dimethylallyl pyrophosphate (DMAPP), e.g., an IPP isomerase. Illustrative examples of nucleotide sequences encoding such an enzyme include: (NC_000913, 3031087.3031635; Escherichia coli), and (AF082326; Haematococcus pluvialis).

### Polyprenyl Synthases

In some embodiments, the host cell further comprises a heterologous nucleotide sequence encoding a polyprenyl synthase that can condense IPP and/or DMAPP molecules to form polyprenyl compounds containing more than five carbons.

The host cell may contain a heterologous nucleotide sequence encoding an enzyme that can condense two molecules of IPP with one molecule of DMAPP, or add a molecule of IPP to a molecule of GPP, to form a molecule of farnesyl pyrophosphate (“FPP”), e.g., a FPP synthase. Non-limiting examples of nucleotide sequences that encode a FPP synthase include: (ATU80605; Arabidopsis thaliana), (ATHFPS2R; Arabidopsis thaliana), (AAU36376; Artemisia annua), (AF461050; Bos taurus), (D00694; Escherichia coli K-12), (AE009951, Locus AAL95523; Fusobacterium nucleatum subsp. nucleatum ATCC 25586), (GFFPPSGEN; Gibberella fujikuroi), (CP000009, Locus AAW60034; Gluconobacter oxydans 621H), (AF019892; Helianthus annuus), (HUMFAPS; Homo sapiens), (KLPFPSQCR; Kluyveromyces lactis), (LAU15777; Lupinus albus), (LAU20771; Lupinus albus), (AF309508; Mus musculus), (NCFPPSGEN; Neurospora crassa), (PAFPS1; Parthenium argentatum), (PAFPS2; Parthenium argentatum), (RATFAPS; Rattus norvegicus), (YSCFPP; Saccharomyces cerevisiae), (D89104; Schizosaccharomyces pombe), (CP000003, Locus AAT87386; Streptococcus pyogenes), (CP000017, Locus AAZ51849; Streptococcus pyogenes), (NC_008022, Locus YP_598856; Streptococcus pyogenes MGAS10270), (NC_008023, Locus YP_600845; Streptococcus pyogenes MGAS2096), (NC_008024, Locus YP_602832; Streptococcus pyogenes MGAS10750), (MZEFPS; Zea mays), (AE000657, Locus AAC06913; Aquifex aeolicus VF5), (NM_202836; Arabidopsis thaliana), (D84432, Locus BAA12575; Bacillus subtilis), (U12678, Locus AAC28894; Bradyrhizobium japonicum USDA 110), (BACFDPS; Geobacillus stearothermophilus), (NC_002940, Locus NP_873754; Haemophilus ducreyi 35000HP), (L42023, Locus AAC23087; Haemophilus influenzae Rd KW20), (J05262; Homo sapiens), (YP_395294; Lactobacillus sakei subsp. sakei 23K), (NC_005823, Locus YP_000273; Leptospira interrogans serovar Copenhageni str. Fiocruz L1-130), (AB003187; Micrococcus luteus), (NC_002946, Locus YP_208768; Neisseria gonorrhoeae FA 1090), (U00090, Locus AAB91752; Rhizobium sp. NGR234), (J05091; Saccharomyces cerevisae), (CP000031, Locus AAV93568; Silicibacter pomeroyi DSS-3), (AE008481, Locus AAK99890; Streptococcus pneumoniae R6), and (NC_004556, Locus NP 779706; Xylella fastidiosa Temecula1).

### Methods of Producing Retinol or Retinol Precursors

The invention provides for the production of retinol or retinol precursor by (a) culturing a population of any of the genetically modified host cells described herein that are capable of producing a retinol or retinol precursor in a medium with a carbon source under conditions suitable for making the retinol or retinol precursor compound, and (b) recovering the retinol or retinol precursor compound from the medium.

The genetically modified host cell produces an increased amount of the retinol or retinol precursor compared to a parent cell not having the genetic modifications, or a parent cell having only a subset of the genetic modifications, but is otherwise genetically identical. In some embodiments, the increased amount is at least 1%, 5%, 10%, 15%, 20%, 25%, 30%, 35%, 40%, 45%, 50%, 55%, 60%, 65%, 70%, 75%, 80%, 85%, 90%, 95%, 100% or greater than 100%, as measured, for example, in yield, production, and/or productivity, in grams per liter of cell culture, milligrams per gram of dry cell weight, on a per unit volume of cell culture basis, on a per unit dry cell weight basis, on a per unit volume of cell culture per unit time basis, or on a per unit dry cell weight per unit time basis.

In some embodiments, the host cell may produce an elevated level of a retinol or retinol precursor that is greater than about 1 gram per liter of fermentation medium. In some embodiments, the host cell produces an elevated level of a retinol or retinol precursor that is greater than about 5 grams per liter of fermentation medium. In some embodiments, the host cell produces an elevated level of a retinol or retinol precursor that is greater than about 10 grams per liter of fermentation medium. In some embodiments, the retinol or retinol precursor is produced in an amount from about 10 to about 50 grams, from about 10 to about 15 grams, more than about 15 grams, more than about 20 grams, more than about 25 grams, or more than about 40 grams per liter of cell culture.

In some embodiments, the host cell produces an elevated level of a retinol or retinol precursor that is greater than about 50 milligrams per gram of dry cell weight. In some such embodiments, the retinol or retinol precursor is produced in an amount from about 50 to about 1500 milligrams, more than about 100 milligrams, more than about 150 milligrams, more than about 200 milligrams, more than about 250 milligrams, more than about 500 milligrams, more than about 750 milligrams, or more than about 1000 milligrams per gram of dry cell weight.

In some embodiments, the host cell produces an elevated level of a retinol or retinol precursor that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2. 5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of retinol or retinol precursor produced by a parent cell, on a per unit volume of cell culture basis.

In some embodiments, the host cell produces an elevated level of a retinol or retinol precursor that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2. 5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of retinol or retinol precursor produced by the parent cell, on a per unit dry cell weight basis.

In some embodiments, the host cell produces an elevated level of a retinol or retinol precursor that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2. 5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of retinol or retinol precursor produced by the parent cell, on a per unit volume of cell culture per unit time basis.

In some embodiments, the host cell produces an elevated level of a retinol or retinol precursor that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2. 5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of retinol or retinol precursor produced by the parent cell, on a per unit dry cell weight per unit time basis.

In most embodiments, the production of the elevated level of retinol or retinol precursor by the host cell is inducible by the presence of an inducing compound. Such a host cell can be manipulated with ease in the absence of the inducing compound. The inducing compound is then added to induce the production of the elevated level of retinol or retinol precursor by the host cell. In other embodiments, production of the elevated level of retinol or retinol precursor by the host cell is inducible by changing culture conditions, such as, for example, the growth temperature, media constituents, and the like.

### Culture Media and Conditions

Materials and methods for the maintenance and growth of microbial cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the host cell, the fermentation, and the process.

The methods of producing retinol or retinol precursor provided herein may be performed in a suitable culture medium (e.g., with or without pantothenate supplementation) in a suitable container, including but not limited to a cell culture plate, a microtiter plate, a flask, or a fermentor. Further, the methods can be performed at any scale of fermentation known in the art to support industrial production of microbial products. Any suitable fermentor may be used including a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric, et al, in Ullmann's Encyclopedia of Industrial Chemistry, Sixth Edition, vol. 12, pp. 398-473, Wiley-VCH Verlag GmbH & Co. KDaA, Weinheim, Germany.

In some embodiments, the culture medium is any culture medium in which a genetically modified microorganism capable of producing a retinol or retinol precursor can subsist. The culture medium may be an aqueous medium comprising assimilable carbon, nitrogen and phosphate sources. Such a medium can also include appropriate salts, minerals, metals, and other nutrients. The carbon source and each of the essential cell nutrients may be added incrementally or continuously to the fermentation media, and each required nutrient may be maintained at essentially the minimum level needed for efficient assimilation by growing cells, for example, in accordance with a predetermined cell growth curve based on the metabolic or respiratory function of the cells which convert the carbon source to a biomass.

Suitable conditions and suitable media for culturing microorganisms are well known in the art. For example, the suitable medium may be supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

The carbon source may be a monosaccharide (simple sugar), a disaccharide, a polysaccharide, a non-fermentable carbon source, or one or more combinations thereof. Non-limiting examples of suitable monosaccharides include glucose, galactose, mannose, fructose, xylose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate and glycerol.

The concentration of a carbon source, such as glucose, in the culture medium may be sufficient to promote cell growth but is not so high as to repress growth of the microorganism used. Typically, cultures are run with a carbon source, such as glucose, being added at levels to achieve the desired level of growth and biomass. The concentration of a carbon source, such as glucose, in the culture medium may be greater than about 1 g/L, preferably greater than about 2 g/L, and more preferably greater than about 5 g/L. In addition, the concentration of a carbon source, such as glucose, in the culture medium is typically less than about 100 g/L, preferably less than about 50 g/L, and more preferably less than about 20 g/L. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include simple nitrogen sources, organic nitrogen sources and complex nitrogen sources. Such nitrogen sources include anhydrous ammonia, ammonium salts and substances of animal, vegetable and/or microbial origin. Suitable nitrogen sources include protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. Beyond certain concentrations, however, the addition of a nitrogen source to the culture medium is not advantageous for the growth of the microorganisms. As a result, the concentration of the nitrogen sources, in the culture medium is less than about 20 g/L, preferably less than about 10 g/L and more preferably less than about 5 g/L. Further, in some instances it may be desirable to allow the culture medium to become depleted of the nitrogen sources during culture.

The effective culture medium may contain other compounds such as inorganic salts, vitamins, trace metals or growth promoters. Such other compounds may also be present in carbon, nitrogen or mineral sources in the effective medium or can be added specifically to the medium.

The culture medium may also contain a suitable phosphate source. Such phosphate sources include both inorganic and organic phosphate sources. Preferred phosphate sources include phosphate salts such as mono or dibasic sodium and potassium phosphates, ammonium phosphate and mixtures thereof. Typically, the concentration of phosphate in the culture medium is greater than about 1.0 g/L, preferably greater than about 2.0 g/L and more preferably greater than about 5.0 g/L. Beyond certain concentrations, however, the addition of phosphate to the culture medium is not advantageous for the growth of the microorganisms. Accordingly, the concentration of phosphate in the culture medium is typically less than about 20 g/L, preferably less than about 15 g/L and more preferably less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, preferably greater than about 1.0 g/L, and more preferably greater than about 2.0 g/L. Beyond certain concentrations, however, the addition of magnesium to the culture medium is not advantageous for the growth of the microorganisms. Accordingly, the concentration of magnesium in the culture medium is typically less than about 10 g/L, preferably less than about 5 g/L, and more preferably less than about 3 g/L. Further, in some instances it may be desirable to allow the culture medium to become depleted of a magnesium source during culture.

The culture medium can also include a biologically acceptable chelating agent, such as the dihydrate of trisodium citrate. In such instance, the concentration of a chelating agent in the culture medium is greater than about 0.2 g/L, preferably greater than about 0.5 g/L, and more preferably greater than about 1 g/L. Beyond certain concentrations, however, the addition of a chelating agent to the culture medium is not advantageous for the growth of the microorganisms. Accordingly, the concentration of a chelating agent in the culture medium is typically less than about 10 g/L, preferably less than about 5 g/L, and more preferably less than about 2 g/L.

The culture medium may also initially include a biologically acceptable acid or base to maintain the desired pH of the culture medium. Biologically acceptable acids include, but are not limited to, hydrochloric acid, sulfuric acid, nitric acid, phosphoric acid and mixtures thereof. Biologically acceptable bases include, but are not limited to, ammonium hydroxide, sodium hydroxide, potassium hydroxide and mixtures thereof. In some embodiments, the base used is ammonium hydroxide.

The culture medium may also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, preferably within the range of from about 20 mg/L to about 1000 mg/L, and more preferably in the range of from about 50 mg/L to about 500 mg/L.

The culture medium may also include sodium chloride. Typically, the concentration of sodium chloride in the culture medium is within the range of from about 0.1 g/L to about 5 g/L, preferably within the range of from about 1 g/L to about 4 g/L, and more preferably in the range of from about 2 g/L to about 4 g/L.

The culture medium may also include trace metals. Such trace metals can be added to the culture medium as a stock solution that, for convenience, can be prepared separately from the rest of the culture medium. Typically, the amount of such a trace metals solution added to the culture medium is greater than about 1 ml/L, preferably greater than about 5 mL/L, and more preferably greater than about 10 mL/L. Beyond certain concentrations, however, the addition of a trace metals to the culture medium is not advantageous for the growth of the microorganisms. Accordingly, the amount of such a trace metals solution added to the culture medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

The culture media may include other vitamins, such as pantothenate, biotin, calcium, pantothenate, inositol, pyridoxine-HCl, and thiamine-HCl. Such vitamins can be added to the culture medium as a stock solution that, for convenience, can be prepared separately from the rest of the culture medium. Beyond certain concentrations, however, the addition of vitamins to the culture medium is not advantageous for the growth of the microorganisms.

The fermentation methods described herein can be performed in conventional culture modes, which include, but are not limited to, batch, fed-batch, cell recycle, continuous and semi-continuous. In some embodiments, the fermentation is carried out in fed-batch mode. In such a case, some of the components of the medium are depleted during culture, including pantothenate during the production stage of the fermentation. In some embodiments, the culture may be supplemented with relatively high concentrations of such components at the outset, for example, of the production stage, so that growth and/or retinol or retinol precursor production is supported for a period of time before additions are required. The preferred ranges of these components are maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those in the art, the rate of consumption of nutrient increases during culture as the cell density of the medium increases. Moreover, to avoid introduction of foreign microorganisms into the culture medium, addition is performed using aseptic addition methods, as are known in the art. In addition, an anti-foaming agent may be added during the culture.

The temperature of the culture medium can be any temperature suitable for growth of the genetically modified cells and/or production of retinol or retinol precursor. For example, prior to inoculation of the culture medium with an inoculum, the culture medium can be brought to and maintained at a temperature in the range of from about 20° C. to about 45° C., preferably to a temperature in the range of from about 25° C. to about 40° C., and more preferably in the range of from about 28° C. to about 32° C. The pH of the culture medium can be controlled by the addition of acid or base to the culture medium. In such cases when ammonium hydroxide is used to control pH, it also conveniently serves as a nitrogen source in the culture medium. Preferably, the pH is maintained from about 3.0 to about 8.0, more preferably from about 3.5 to about 7.0, and most preferably from about 4.0 to about 6.5.

The carbon source concentration, such as the glucose concentration, of the culture medium is monitored during culture. Glucose concentration of the culture medium can be monitored using known techniques, such as, for example, use of the glucose oxidase enzyme test or high pressure liquid chromatography, which can be used to monitor glucose concentration in the supernatant, e.g., a cell-free component of the culture medium. The carbon source concentration is typically maintained below the level at which cell growth inhibition occurs. Although such concentration may vary from organism to organism, for glucose as a carbon source, cell growth inhibition occurs at glucose concentrations greater than at about 60 g/L, and can be determined readily by trial. Accordingly, when glucose is used as a carbon source the glucose is preferably fed to the fermentor and maintained below detection limits. Alternatively, the glucose concentration in the culture medium is maintained in the range of from about 1 g/L to about 100 g/L, more preferably in the range of from about 2 g/L to about 50 g/L, and yet more preferably in the range of from about 5 g/L to about 20 g/L. Although the carbon source concentration can be maintained within desired levels by addition of, for example, a substantially pure glucose solution, it is acceptable, and may be preferred, to maintain the carbon source concentration of the culture medium by addition of aliquots of the original culture medium. The use of aliquots of the original culture medium may be desirable because the concentrations of other nutrients in the medium (e.g. the nitrogen and phosphate sources) can be maintained simultaneously. Likewise, the trace metals concentrations can be maintained in the culture medium by addition of aliquots of the trace metals solution.

Other suitable fermentation medium and methods are described in, e.g., WO 2016/196321, which is incorporated herein by reference in its entirety.

### Recovery of Retinol or Retinol Precursors

Once the retinol or retinol precursor is produced by the host cell, it may be recovered or isolated for subsequent use using any suitable separation and purification methods known in the art. For example, a clarified aqueous phase, emulsion, or oil phase containing the retinol or retinol precursor may be separated from the fermentation by centrifugation. Alternatively, a clarified aqueous phase, emulsion, or oil phase containing the retinol or retinol precursor may be separated from the fermentation by adding a demulsifier into the fermentation reaction. Examples of demulsifiers include flocculants and coagulants.

The retinol or retinol precursor produced in the host cells may be present in the culture supernatant and/or associated with the host cells. Where some of the retinol or retinol precursor is associated with the host cell, the recovery of the retinol or retinol precursor may involve a method of improving the release of the retinol or retinol precursor from the cells. This could take the form of washing the cells with hot water or buffer treatment, with or without a surfactant, and with or without added buffers or salts. The temperature may be any temperature deemed suitable for releasing the retinol or retinol precursor. For example, the temperature may be in a range from 40 to 95° C.; or from 60 to 90° C.; or from 75 to 85° C. Alternatively, the temperature may be 40, 45, 50, 55, 65, 70, 75, 80, 85, 90, or 95° C. Physical or chemical cell disruption may be used to enhance the release of retinol or retinol precursor from the host cell. Alternatively, and/or subsequently, the retinol or retinol precursor in the culture medium may be recovered using an isolation-unit operations including, solvent extraction, membrane clarification, membrane concentration, adsorption, chromatography, evaporation, chemical derivatization, crystallization, and drying.

### Methods of Making Genetically Modified Cells

Also provided herein are methods for producing a host cell that is genetically engineered to contain one or more of the modifications described above, e.g., one or more heterologous nucleic acids encoding phytoene synthase, lycopene desaturase, lycopene cyclase, BCDO and/or RDH, and/or biosynthetic pathway enzymes, e.g., for a retinol or retinol precursor compound. Expression of a heterologous enzyme in a host cell can be accomplished by introducing into the host cells a nucleic acid comprising a nucleotide sequence encoding the enzyme under the control of regulatory elements that permit expression in the host cell. The nucleic acid may be an extrachromosomal plasmid, a chromosomal integration vector that can integrate the nucleotide sequence into the chromosome of the host cell, or a linear piece of double stranded DNA that can integrate via homology the nucleotide sequence into the chromosome of the host cell.

Nucleic acids encoding these proteins can be introduced into the host cell by any method known to one of skill in the art (see, e.g., Hinnen et al., (1978) Proc. Natl. Acad. Sci. USA, vol. 75, pp. 1292-1293; Cregg et al., (1985), Mol. Cell. Biol., vol. 5, pp. 3376-3385; Goeddel et al. eds, 1990, Methods in Enzymology, vol. 185, Academic Press, Inc., CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, NY; and Ausubel et al., eds., Current Edition, Current Protocols in Molecular Biology, Greene Publishing Associates and Wiley Interscience, NY). Exemplary techniques include, spheroplasting, electroporation, PEG 1000 mediated transformation, and lithium acetate or lithium chloride mediated transformation.

The amount of an enzyme in a host cell may be altered by modifying the transcription of the gene that encodes the enzyme. This can be achieved by modifying the copy number of the nucleotide sequence encoding the enzyme (e.g., by using a higher or lower copy number expression vector comprising the nucleotide sequence, or by introducing additional copies of the nucleotide sequence into the genome of the host cell or by deleting or disrupting the nucleotide sequence in the genome of the host cell), by changing the order of coding sequences on a polycistronic mRNA of an operon or breaking up an operon into individual genes each with its own control elements, or by increasing the strength of the promoter or operator to which the nucleotide sequence is operably linked. Alternatively, or in addition, the copy number of an enzyme in a host cell may be altered by modifying the level of translation of an mRNA that encodes the enzyme. This can be achieved by modifying the stability of the mRNA, modifying the sequence of the ribosome binding site, modifying the distance or sequence between the ribosome binding site and the start codon of the enzyme coding sequence, modifying the entire intercistronic region located “upstream of” or adjacent to the 5′ side of the start codon of the enzyme coding region, stabilizing the 3′-end of the mRNA transcript using hairpins and specialized sequences, modifying the codon usage of enzyme, altering expression of rare codon tRNAs used in the biosynthesis of the enzyme, and/or increasing the stability of the enzyme, as, for example, via mutation of its coding sequence.

The activity of an enzyme in a host cell may be altered in a number of ways, including expressing a modified form of the enzyme that exhibits increased or decreased solubility in the host cell, expressing an altered form of the enzyme that lacks a domain through which the activity of the enzyme is inhibited, expressing a modified form of the enzyme that has a higher or lower K_{cat} or a lower or higher Kₘ for the substrate, expressing a modified form of the enzyme that has a higher or lower thermostability, expressing a modified form of the enzyme that has a higher or lower activity at the pH of the cell, expressing a modified form of the enzyme that has a higher or lower accumulation in a subcellular compartment or organelle, expressing a modified form of the enzyme that has increased or decreased ability to insert into or associate with cellular membranes, expressing a modified form of the enzyme that has a higher or lower affinity for accessory proteins needed to carry out a reaction, expressing a modified form of the enzyme that has a higher or lower affinity for necessary cofactors or ligands, expressing a modified form of the enzyme that has a increased or decreased space in the active site (thereby differentially allowing or excluding different substrates for the reaction), or expressing an altered form of the enzyme that is more or less affected by feed-back or feed-forward regulation by another molecule in the pathway.

A nucleic acid used to genetically modify a host cell may contain one or more selectable markers useful for the selection of transformed host cells and for placing selective pressure on the host cell to maintain the foreign DNA.

The selectable marker may be an antibiotic resistance marker. Examples of antibiotic resistance markers include the BLA, NAT1, PAT, AUR1-C, PDR4, SMR1, CAT, mouse dhfr, HPH, DSDA, KAN^{R}, and SH BLE gene products. The BLA gene product from E. coli confers resistance to beta-lactam antibiotics (e.g., narrow-spectrum cephalosporins, cephamycins, and carbapenems (ertapenem), cefamandole, and cefoperazone) and to all the anti-gram-negative-bacterium penicillins except temocillin; the NAT1 gene product from S. noursei confers resistance to nourseothricin; the PAT gene product from S. viridochromogenes Tu94 confers resistance to bialophos; the AUR1-C gene product from Saccharomyces cerevisiae confers resistance to Auerobasidin A (AbA); the PDR4 gene product confers resistance to cerulenin; the SMR1 gene product confers resistance to sulfometuron methyl; the CAT gene product from Tn9 transposon confers resistance to chloramphenicol; the mouse dhfr gene product confers resistance to methotrexate; the HPH gene product of Klebsiella pneumonia confers resistance to Hygromycin B; the DSDA gene product of E. coli allows cells to grow on plates with D-serine as the sole nitrogen source; the KAN^{R} gene of the Tn903 transposon confers resistance to G418; and the SH BLE gene product from Streptoalloteichus hindustanus confers resistance to Zeocin (bleomycin). The antibiotic resistance marker may be deleted after the genetically modified host cell disclosed herein is isolated.

The selectable marker may function by rescue of an auxotrophy (e.g., a nutritional auxotrophy) in the genetically modified microorganism. In auxotrophy, a parent microorganism contains a functional disruption in one or more gene products that function in an amino acid or nucleotide biosynthetic pathway and that renders the parent cell incapable of growing in media without supplementation with one or more nutrients. Such gene products include the HIS3, LEU2, LYS1, LYS2, MET15, TRP1, ADE2, and URA3 gene products in yeast. The auxotrophic phenotype can then be rescued by transforming the parent cell with an expression vector or chromosomal integration construct encoding a functional copy of the disrupted gene product, and the genetically modified host cell generated can be selected for based on the loss of the auxotrophic phenotype of the parent cell. Utilization of the URA3, TRP1, and LYS2 genes as selectable markers has a marked advantage because both positive and negative selections are possible. Positive selection is carried out by auxotrophic complementation of the URA3, TRP1, and LYS2 mutations, whereas negative selection is based on specific inhibitors, i.e., 5-fluoro-orotic acid (FOA), 5-fluoroanthranilic acid, and aminoadipic acid (aAA), respectively, that prevent growth of the prototrophic strains but allows growth of the URA3, TRP1, and LYS2 mutants, respectively. The selectable marker may rescue other non-lethal deficiencies or phenotypes that can be identified by a known selection method.

Described herein are specific genes and proteins useful in the methods, compositions, and host cells of the invention; however, the absolute identity to such genes is not necessary. For example, changes in a particular gene or polynucleotide containing a sequence encoding a polypeptide or enzyme can be performed and screened for activity. Typically, such changes involve conservative mutations and silent mutations. Such modified or mutated polynucleotides and polypeptides can be screened for expression of a functional enzyme using methods known in the art.

Due to the inherent degeneracy of the genetic code, other polynucleotides which encode substantially the same or functionally equivalent polypeptides may also be used to express the enzymes.

It can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.” Codon optimization for other host cells can be readily determined using codon usage tables or can be performed using commercially available software, such as CodonOp from Integrated DNA Technologies.

Optimized coding sequences containing codons preferred by a particular prokaryotic or eukaryotic host (Murray et al., (1989), Nucl Acids Res., vol. 17, pp. 477-508) can be prepared, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., (1996), Nucl Acids Res., vol. 24, pp. 216-218).

Due to the degenerate nature of the genetic code, a variety of DNA molecules differing in their nucleotide sequences may be used to encode a given enzyme of the disclosure. The native DNA sequence encoding the biosynthetic enzymes described above are referenced herein merely to illustrate an embodiment of the disclosure, and the disclosure includes DNA molecules of any sequence that encode the amino acid sequences of the polypeptides and proteins of the enzymes utilized in the methods of the invention. In similar fashion, a polypeptide can typically tolerate one or more amino acid substitutions, deletions, and insertions in its amino acid sequence without loss or significant loss of a desired activity. The invention includes such polypeptides with different amino acid sequences than the specific proteins described herein so long as the modified or variant polypeptides have the enzymatic activity of the reference polypeptide. Furthermore, the amino acid sequences encoded by the DNA sequences shown herein merely illustrate examples of the invention.

In addition, homologs of enzymes useful for the practice of the compositions, methods, or host cells are encompassed by the invention. Two proteins (or a region of the proteins) are considered to be substantially homologous when the amino acid sequences have at least about 30%, 40%, 50%, 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes may be at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (See, e.g., Pearson W. R., (1994), Methods in Mol Biol, vol. 25, pp. 365-389).

The following six groups each contain amino acids that are conservative substitutions for one another: 1) Serine(S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. A typical algorithm used for comparing a molecule sequence to a database containing a large number of sequences from different organisms is the computer program BLAST. When searching a database containing sequences from a large number of different organisms, it is typical to compare amino acid sequences.

Furthermore, any of the genes encoding the foregoing enzymes or any of the regulatory elements that control or modulate their expression may be optimized by genetic/protein engineering techniques, such as directed evolution or rational mutagenesis. Such action allows those of ordinary skill in the art to optimize the enzymes for expression and activity in yeast.

In addition, genes encoding these enzymes can be identified from other fungal and bacterial species and can be expressed for the modulation of the retinol or retinol precursor pathway. A variety of organisms may serve as sources for these enzymes, including Saccharomyces spp., including S. cerevisiae and S. uvarum, Kluyveromyces spp., including K. thermotolerans, K. lactis, and K. marxianus, Pichia spp., Hansenula spp., including H. polymorpha, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include Piromyces spp., Orpinomyces spp., or Neocallimastix spp. Sources of prokaryotic enzymes that are useful include Escherichia. coli, Zymomonas mobilis, Staphylococcus aureus, Bacillus spp., Clostridium spp., Corynebacterium spp., Pseudomonas spp., Lactococcus spp., Enterobacter spp., and Salmonella spp.

Techniques known to those skilled in the art may be suitable to identify additional homologous genes and enzymes. Generally, analogous genes and/or analogous enzymes can be identified by functional analysis and will have functional similarities. Techniques known to be suitable to identify analogous genes and analogous enzymes include PCR, degenerate PCR, low stringency nucleic acid hybridization, expression cloning, and high through-put screening. For example, to identify homologous or analogous terpene synthase, or any retinol or retinol precursor biosynthetic pathway genes, proteins, or enzymes, techniques may include, but are not limited to, cloning a gene by PCR using primers based on a published sequence of a gene/enzyme of interest, or by degenerate PCR using degenerate primers designed to amplify a conserved region among a gene of interest. Further, one may use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K., Branched-Chain Amino Acids Methods Enzymology, 1970), then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, or MetaCYC. The candidate gene or enzyme may be identified within the above-mentioned databases in accordance with the teachings herein.

### EXAMPLES

### Example 1: General Transformation Protocol

Each DNA construct was integrated into Saccharomyces cerevisiae (CEN.PK113-7D) using standard molecular biology techniques in an optimized lithium acetate transformation. Briefly, cells were grown overnight in yeast extract peptone dextrose (YPD) medium at 30° C. with shaking (200 rpm), diluted to an OD₆₀₀ of 0.1 in 100 mL YPD, and grown to an OD₆₀₀ of 0.6-0.8. For each transformation, 5 mL of culture were harvested by centrifugation, washed in 5 mL of sterile water, spun down again, resuspended in 1 mL of 100 mM lithium acetate, and transferred to a microcentrifuge tube. Cells were spun down (13,000×g) for 30 seconds, the supernatant was removed, and the cells were resuspended in a transformation mix consisting of 240 μL 50% PEG, 36 μL 1 M lithium acetate, 10 μL boiled salmon sperm DNA, and 74 μL of donor DNA. In some cases, the donor DNA included a plasmid carrying the endonuclease gene in such a manner cuts a specific recognition site engineered in a host strain to facilitate integration of the target gene of interest. Alternatively, the transformation can be performed using donor DNA and a plasmid carrying a gRNA as described by Walters et. al. Following a heat shock at 42° C. for 40 minutes, cells were recovered overnight in YPD medium before plating on selective medium. DNA integration was confirmed by colony PCR with primers specific to the integrations.

### Example 2: General Yeast Culture Protocol

For routine strain characterization in a 96-well-plate format, yeast colonies were picked into a 1.1-mL per well capacity 96-well ‘Pre-Culture plate’ filled with 360 μL per well of pre-culture medium. Pre-culture medium consists of Bird Seed Media (BSM, originally described by van Hoek et al., Biotech, and Bioengin., 68, 2000, 517-23) at pH 5.05 with 14 g/L sucrose, 7 g/L maltose, 3.75 g/L ammonium sulfate, and 1 g/L lysine. Cells were cultured at 28° C. in a high capacity microtiter plate incubator shaking at 1000 rpm and 80% humidity for 3 days until the cultures reached carbon exhaustion.

The growth-saturated cultures were sub-cultured by taking 14.4 μL from the saturated cultures and diluting into a 2.2 mL per well capacity 96-well ‘production plate’ filled with 360 μL per well of production medium. Production medium consisted of BSM at pH 5.05 with 40 g/L sucrose, 3.75 g/L ammonium sulfate and 25% v/v of IPM (isopropyl-myristate). Cells in the production medium were cultured at 30° C. in a high-capacity microtiter plate incubator shaking at 1000 rpm and 80% humidity for an additional 3 days prior to extraction and analysis.

### Example 3: Analytical Methods for Product Extraction and Titer Determination

After incubation of the production plate, methanol and ethyl acetate were added, the plate was sealed, then shaken at 1500 rpm for 30 minutes to lyse cells and extract the retinoids. The plate was centrifuged for 5 minutes at 2000 rpm to pellet cell debris. From the production plate, 400 μL of the supernatant was transferred to an empty 1.1 mL 96-well plate and sealed. The sample plate was then stored at −20° C. until analysis.

Sample plates were analyzed for retinal and retinol titer on a weight by volume basis through external calibration with authentic standards. Measurements were performed by ultra-high pressure liquid chromatography and ultraviolet detection (UPLC-UV) using an Agilent 1290 Infinity II UHPLC system with an Agilent 1290 Infinity II Diode Array Detector. The peak areas of retinol and the heights of retinal were used to generate the linear calibration curve for the respective analyte.

TABLE 1
Mobile Phases and Column Information

| | |
|---|---|
| Mobile Phase A | Methanol + 10 mg/L Potassium Hydroxide |
| Mobile Phase B | Isopropanol |
| Wash Solvent | 80% MilliQ water + 20% Methanol |
| Column | Phenomenex Luna 3 μm C18 100 Å, |
| | 250 × 4.6 mm P/N: 00G-4251-E0 |
| Guard Column | Phenomenex SecurityGuard Cartridge for |
| | C18 column P/N: AJ0-4287 |
| Inline Filter | Agilent 0.3 μm Inline Filter P/N: 5023-0271 |

TABLE 2
Mobile Phase Gradient

| Time | Flow (mL/min) | % A | % B | Curve |
|---|---|---|---|---|
| 0 | 1.0 | 100 | 0 | 5 |
| 5.2 | 1.0 | 100 | 0 | 5 |
| 5.8 | 1.0 | 60 | 40 | 5 |
| 6.0 | 1.0 | 60 | 40 | 5 |
| 6.6 | 1.0 | 100 | 0 | 5 |
| 7.5 | 1.0 | 100 | 0 | 5 |

TABLE 3
Column Compartment Settings

| | |
|---|---|
| Temperature Control: | On |
| Temperature: | 45° C. |
| Temperature Range: | Within ± 0.8° C. for 15 min |
| Equilibration Time: | 15 min |

TABLE 4
Multisampler Settings

| | |
|---|---|
| Needle Wash | Standard Wash |
| Posttime | Off |
| Draw Speed | 100.0 μL/min |
| Eject Speed | 200.0 μL/min |
| Wait Time After Draw | 1.0 s |
| Needle Height Position | Offset 1.0 nm |
| Cooler | On, 4° C. |

TABLE 5
Detector Settings

| Wavelength | 340 | nm |
|---|---|---|
| Bandwidth | 4.0 | nm |
| Peakwidth | >0.013 min (0.25 s response time) *20 Hz) | |
| Posttime | Off | |
| Spectrum | Store: All, Range from 190.0 to 400 nm, Step: | |
| | 2.0 nm | |
| Analog Output | Zero Offset: 5%, Attenuation: 1000 mAU | |
| Margin for Negative | 100 | mAU |
| Absorbance | | |
| Slit | 4 | nm |

| | |
|---|---|
| Autobalance | Prerun |
| Lamps on required | UV Lamp |
| for acquisition | |

### Example 4: Analytical Methods for Product Extraction and Relative Strain Ranking

After incubation of the production plate, 456 μL isopropanol was added, the plate was shaken at 1000 rpm for 90 seconds to lyse cells and extract the retinoids. After shaking, 144 μL of water was added, the plate was sealed, then shaken at 1000 rpm for 20 seconds. The plate was centrifuged for 15 seconds at 500×g to pellet cell debris. From the production plate, 8 μL of the supernatant was transferred to a new Greiner 655801 clear flat bottom plate containing 144 μL isopropanol and 48 μL water. The assay plate was shaken for 1 minute at 900 rpm and transferred to Spectrophotometer for analysis. Sample plates were analyzed at 324 nm to measure retinol and the absorbance values were used as relative retinol amounts in each of the wells.

### Example 5: Generation of a Base Yeast Strain Capable of High Flux to Farnesyl-Pyrophosphate (FPP) and the Isoprenoid Farnesene

A farnesene production strain was created from a wild-type Saccharomyces cerevisiae strain (CEN.PK2) by expressing the genes of the mevalonate pathway under the control of GAL1 or GAL10 promoters. This strain comprised the following chromosomally integrated mevalonate pathway genes from S. cerevisiae: acetyl-CoA thiolase (SEQ ID NO: 1), HMG-COA synthase (SEQ ID NO: 2), HMG-COA reductase (SEQ ID NO: 3 and SEQ ID NO: 4), phosphomevalonate kinase (SEQ ID NO: 6), mevalonate pyrophosphate decarboxylase (SEQ ID NO: 7), IPP: DMAPP isomerase (SEQ ID NO: 8), and farnesyl pyrophosphate synthase (SEQ ID NO: 5). In addition, the strain contained six copies of farnesene synthase from Artemisinin annua, also under the control of either GAL1 or GAL10 promoters. The strains also contain an ERG9 gene, encoding squalene synthase, which was downregulated by replacing the native promoter with promoter of the yeast gene MET3 (Westfall et al PNAS 2012). Examples of methods for creating S. cerevisiae strains with high flux to FPP are described in the U.S. Pat. No. 8,415,136 which are incorporated herein in their entireties.

### Example 6: Generation of a Base Strain for Retinol Dehydrogenase (RDH) Screening

To convert the farnesene base strain described in Example 5 to have high flux to the C-20 isoprenoid retinol, 2 copies of a geranylgeranylpyrophosphate synthase (GGPPS) (SEQ ID NO: 9) were integrated into the genome, followed by one copy of the gene encoding a bi-functional enzyme (CrtYB) (SEQ ID NO: 10) with phytoene synthase and lycopene cyclase activity, one copy of lycopene desaturase (CrtI) (SEQ ID NO: 11), three copies of beta-carotene-15′,15′-dioxygenase (Hs.BCDO) (SEQ ID NO: 12) and deletion of one native aldehyde dehydrogenase (ADH6) (SEQ ID NO: 13) (Table 6). At this point the six copies of farnesene synthase were removed from the strain. The screening strain primarily produced retinal and was capable of producing retinol in the presence of active retinol dehydrogenase (RDH) enzymes.

To measure the activity of RDHs in vivo in S. cerevisiae, a landing pad was introduced into this screening strain, which allowed for the rapid insertion of RDH variants. The landing pad consisted of 500 bp of locus-targeting DNA sequences on either end of the construct to the genomic region downstream of the yeast locus of choice (Upstream locus and Downstream locus), thereby integrating the new sequence in the chromosome of the base strain. Internally, the landing pad contained a promoter which could be GAL1, GAL3 or any other promoter of the yeast GAL regulon, and a yeast native terminator of choice flanking an endonuclease recognition site. DNA variants of the RDH library were used to transform the strain along with a plasmid expressing endonuclease, which created a double strand break at the recognition sequence and facilitated homologous recombination of the DNA variants at the site. At least six colonies from each transformation were used to screen for RDH activity, using methods described in Example 2 and Example 3.

TABLE 6
List of Genes, and Copy Number Used to Convert FPP into
Base Strains to Screen for RDH and BCDO Variants

| Enzyme | Copy | |
|---|---|---|
| Name | Number | SEQ ID NO: |
| Bt. GGPPS | 2 | SEQ ID NO: 9 |
| Xd. CrtYB | 1 | SEQ ID NO: 10 |
| Xd. CrtI | 1 | SEQ ID NO: 11 |
| Hs. BCDO | 3 | SEQ ID NO: 12 |
| Sc. ADH6 | 0 | SEQ ID NO: 13 |
| Dr. RDH8 | 1 | SEQ ID NO: 51 |

*Note: SEQ ID NO: 51 was used in Example 8

### Example 7: Identification of Novel Proteins with RDH Activity from a Natural Diversity Search

Retinol dehydrogenases catalyze the conversion of retinal to retinol. A library of candidate protein sequences was assembled by performing homology searching with three different query sequences. The three query sequences were chosen based on literature reports of either confirmed or probable retinal reductase activity. Two were retinal reductases from Homo sapiens, Hs.RDH12 (PubMed: 15865448, PubMed: 12226107) and Hs.RDH8 (https://pubmed.ncbi.nlm.nih.gov/10753906/). One was an alcohol dehydrogenase from S. cerevisiae with broad substrate specificity ADH6.

Each query sequence was used to perform basic local alignment search tool against the Eggnog database (Nucleic Acids Res. 2019 Jan. 8; 47 (Database issue): D309-D314. doi: 10.1093/nar/gky1085) restricted to sequences derived from fish, birds and reptile species. Ultimately, 74 protein sequences were codon-optimized for S. cerevisiae and ordered from a DNA-synthesis vendor.

Codon-optimized sequences were incorporated into the S. cerevisiae strain of Example 6 and screened for conversion of retinal into retinol.

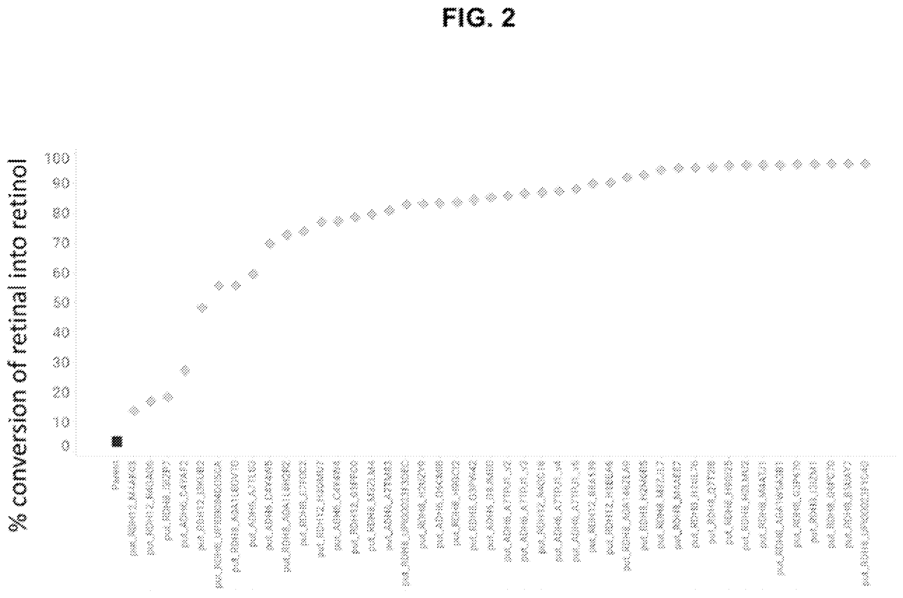

Out of the 74 proteins screened, 41 proteins (SEQ ID NOS: 14-54) produced retinol at least one standard deviation higher than the screening strain and were classified as hits. These 41 proteins converted 15% to 100% of retinal into retinol (FIG. 2 and Table 7).

TABLE 7
List of Retinol Dehydrogenases Producing Retinol At Least
One Standard Deviation Higher Than the Screening Strain

| | % Conversion | |
|---|---|---|
| | of Retinal | |
| Source | to Retinol | SEQ ID NO: |
| Vanderwaltozyma polyspora | 59.9 | SEQ ID NO: 14 |
| Vanderwaltozyma polyspora | 81.0 | SEQ ID NO: 15 |
| Vanderwaltozyma polyspora | 86.1 | SEQ ID NO: 16 |
| Clavispora lusitaniae | 77.5 | SEQ ID NO: 17 |
| Clavispora lusitaniae | 70.0 | SEQ ID NO: 18 |
| Clavispora lusitaniae | 27.5 | SEQ ID NO: 19 |
| Eremothecium cymbalariae | 85.5 | SEQ ID NO: 20 |
| Kluyveromyces lactis | 83.5 | SEQ ID NO: 21 |
| Danio rerio | 90.1 | SEQ ID NO: 22 |
| Gasterosteus aculeatus | 78.7 | SEQ ID NO: 23 |
| Latimeria chalumnae | 90.5 | SEQ ID NO: 24 |
| Tetraodon nigroviridis | 77.4 | SEQ ID NO: 25 |
| Oreochromis niloticus | 48.6 | SEQ ID NO: 26 |
| Xiphophorus maculatus | 14.0 | SEQ ID NO: 27 |
| Anolis carolinensis | 17.4 | SEQ ID NO: 28 |
| Anolis carolinensis | 87.1 | SEQ ID NO: 29 |
| Fundulus heteroclitus | 92.2 | SEQ ID NO: 30 |
| Xenopus laevis | 56.2 | SEQ ID NO: 31 |
| Xenopus laevis | 73.0 | SEQ ID NO: 32 |
| Scleropages formosus | 96.4 | SEQ ID NO: 33 |
| Cyprinus carpio | 96.7 | SEQ ID NO: 34 |
| Danio rerio | 74.2 | SEQ ID NO: 35 |
| Gasterosteus aculeatus | 96.4 | SEQ ID NO: 36 |
| Gasterosteus aculeatus | 84.6 | SEQ ID NO: 37 |
| Oryzias latipes | 96.3 | SEQ ID NO: 38 |
| Oryzias latipes | 93.0 | SEQ ID NO: 39 |
| Fugu rubripes | 95.4 | SEQ ID NO: 40 |
| Takifugu rubripes | 83.4 | SEQ ID NO: 41 |
| Anolis carolinensis | 83.9 | SEQ ID NO: 42 |
| Anolis carolinensis | 96.0 | SEQ ID NO: 43 |
| Oreochromis niloticus | 96.5 | SEQ ID NO: 44 |
| Oreochromis niloticus | 18.6 | SEQ ID NO: 45 |
| Xiphophorus maculatus | 94.6 | SEQ ID NO: 46 |
| Xiphophorus maculatus | 79.9 | SEQ ID NO: 47 |
| Xiphophorus maculatus | 95.2 | SEQ ID NO: 48 |
| Xiphophorus maculatus | 96.3 | SEQ ID NO: 49 |
| Danio rerio | 96.7 | SEQ ID NO: 50 |
| Danio rerio | 95.6 | SEQ ID NO: 51 |
| Gadus morhua | 96.7 | SEQ ID NO: 52 |
| Gadus morhua | 83.3 | SEQ ID NO: 53 |
| Xenopus laevis | 55.9 | SEQ ID NO: 54 |

### Example 8: Generation of a Base Strain for BCDO Screening

To convert the farnesene base strain described in Example 5 to have high flux to the C-20 isoprenoid retinol, 2 copies of a geranylgeranylpyrophosphate synthase (GGPPS) (SEQ ID NO: 9) were integrated into the genome, followed by one copy of the gene encoding a bi-functional enzyme (CrtYB) (SEQ ID NO: 10) with phytoene synthase and lycopene cyclase activity, one copy of lycopene desaturase (CrtI) (SEQ ID NO: 11) and one copy of retinol dehydrogenase (RDH8) (SEQ ID NO: 51) (Table 6). At this point the six copies of farnesene synthase were removed from the strain. The strain containing all genes described in Table 6 primarily produced beta-carotene and was capable of producing retinol in the presence of active BCDO enzymes.

To measure the activity of BCDOs in vivo in S. cerevisiae, a landing pad was introduced into this screening strain, which allowed for the rapid insertion of BCDO variants. The landing pad consisted of 500 bp of locus-targeting DNA sequences on either end of the construct to the genomic region downstream of the yeast locus of choice (Upstream locus and Downstream locus), thereby integrating the new sequence in the chromosome of the base strain. Internally, the landing pad contained a promoter which could be GAL1, GAL3 or any other promoter of the yeast GAL regulon, and a yeast native terminator of choice flanking an endonuclease recognition site. DNA variants of the BCDO library were used to transform the strain along with a plasmid expressing endonuclease, which created a double strand break at the recognition sequence and facilitated homologous recombination of the DNA variants at the site. At least six colonies from each transformation were used to screen for BCDO activity, using methods described in Example 2 and Example 3.

### Example 9: Identification of Novel Proteins with BCDO Activity from a Natural Diversity Search

Beta-carotene 15-15′-dioxygenases catalyze the conversion of beta-carotene to retinal. A library of candidate protein sequences was assembled by performing homology searching with 7 different query sequences. The 7 query sequences were chosen based on literature reports of either confirmed or probable 15-15′-beta carotene dioxygenase (BCDO) activity. A likely BCDO was identified by genomic analysis of the fungus Zymoseptoria tritici (SEQ ID NO: 60) (Cairns and Meyer, BMC Genomics, 2017, 18:631). A BCDO was identified biochemically from the fungus Fusarium fujikuroi (SEQ ID NO: 110) (Prado-Cabrero et al, Eukaryotic Cell, 2007 April, p. 650-657). A BCDO was identified biochemically from the fungus Ustilago maydis (SEQ ID NO: 56). A BCDO was identified biochemically from the uncultured marine bacterium 66A03 (SEQ ID NO: 149) (Kim et al, J Biol. Chem., 2009, 284 (23): 15781-15793). A likely BCDO was identified using functional genomics from the marine bacterium Dokdonia MED134 (SEQ ID NO: 57) (Kimura et al, ISME J., 2011, 5 (10): 1641-1651). A likely BCDO was identified by heterologous pathway reconstruction from a freshwater bacterium Actinobacterium SCGC AAA278-022 (SEQ ID NO: 58) (Dwulit-Smith et al, Appl. Environ. Microbiol., 2018, 84 (24): e01678-18). A likely BCDO was identified by heterologous pathway reconstruction from the halophilic bacterium Salinibacter ruber (SEQ ID NO: 59) (Choi et al, Antioxidants (Basel), 2020, 9 (11): 1130). Each query sequence was used to perform three iterations of position specific iterative basic local alignment search tool (PSI-BLAST, Altschul et al., Nuc. Acid Research, 25:17, 1997, 3389-3402) against a pre-clustered protein database (UniRef90, Baris et al, Bioinformatics, 31:6, 2015, 926-32). The resultant position specific scoring matrix (PSSM) was used to query all known protein sequences stored by the National Center for Biotechnology Information (NCBI-nr/RefSeq non-redundant) resulting in several thousand amino acid sequences each. Sequences were clustered based on pairwise amino acid similarity using CD-HIT (https://doi.org/10.1093/bioinformatics/bt1158), and candidate sequences were chosen manually from the resultant clusters to add to the library. Ultimately, 148 protein sequences were codon-optimized for S. cerevisiae and ordered from a DNA-synthesis vendor.

This library of genes was then screened in an engineered S. cerevisiae strain described in Example 8. Generation of a base strain for BCDO screening. The immediate product of the BCDO is retinal, but retinol was used as a primary readout for BCDO activity, as a functional BCDO increases retinal production; downstream enzyme RDH8 was not limiting in this screening strain.

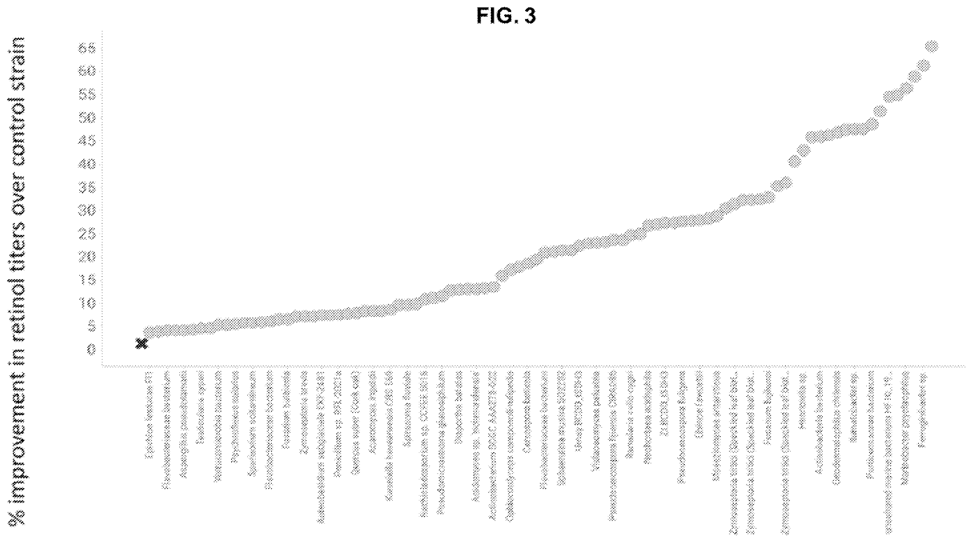

Out of 148 proteins screened, 94 produced retinol at least one standard deviation higher than the screening strain and were classified as hits (FIG. 3 and Table 8) (SEQ ID NOs: 55-148).

TABLE 8
List of BCDOs Producing Retinol At Least One Standard
Deviation Higher Than the Screening Strain

| | % Improvement Over | |
|---|---|---|
| Source | Screening Strain | SEQ ID NO: |
| Gibberella fujikuroi | 25.0 | SEQ ID NO: 55 |
| Ustilago maydis (strain | 22.6 | SEQ ID NO: 56 |
| 521/FGSC 9021) | | |
| Dokdonia sp. MED134 | 11.7 | SEQ ID NO: 57 |
| Actinobacterium SCGC | 13.7 | SEQ ID NO: 58 |
| AAA278-22 | | |
| Salinibacter ruber | 21.6 | SEQ ID NO: 59 |
| Zymoseptoria tritici | 27.5 | SEQ ID NO: 60 |
| ST99CH 3D7 | | |
| Planctomycetes bacterium | 46.4 | SEQ ID NO: 61 |
| Halieaceae bacterium | 58.9 | SEQ ID NO: 62 |
| Porticoccaceae bacterium | 48.7 | SEQ ID NO: 63 |
| Gammaproteobacteria | 9.9 | SEQ ID NO: 64 |
| bacterium | | |
| Euryarchaeota archaeon | 51.6 | SEQ ID NO: 65 |
| Alphaproteobacteria | 65.5 | SEQ ID NO: 66 |
| bacterium | | |
| uncultured marine bacterium | 54.7 | SEQ ID NO: 67 |
| HF10_19P19 | | |
| Henriciella sp. | 43.1 | SEQ ID NO: 68 |
| Cryomorphaceae bacterium | 4.8 | SEQ ID NO: 69 |
| BACL22 MAG-120619- | | |
| bin32 | | |
| Loktanella sp. SALINAS62 | 5.9 | SEQ ID NO: 70 |
| Flavobacteriaceae | 4.2 | SEQ ID NO: 71 |
| bacterium | | |
| Robertkochia sp. 3YJGBD- | 7.3 | SEQ ID NO: 72 |
| 33 | | |
| Flavobacteriaceae | 6.3 | SEQ ID NO: 73 |
| bacterium | | |
| Eudoraea sp. | 4.1 | SEQ ID NO: 74 |
| Psychroflexus salarius | 5.6 | SEQ ID NO: 75 |
| Verrucomicrobia bacterium | 5.5 | SEQ ID NO: 76 |
| Flavobacteriaceae | 21.1 | SEQ ID NO: 77 |
| bacterium | | |
| Sphingomonadales | 7.9 | SEQ ID NO: 78 |
| bacterium | | |
| Ferruginibacter sp. | 61.5 | SEQ ID NO: 79 |
| Actinobacteria bacterium | 46.2 | SEQ ID NO: 80 |
| Spirosoma fluviale | 9.8 | SEQ ID NO: 81 |
| Actinobacteria bacterium | 55.1 | SEQ ID NO: 82 |
| Marinobacter psychrophilus | 56.4 | SEQ ID NO: 83 |
| Actinomycetales bacterium | 47.6 | SEQ ID NO: 84 |
| Geodermatophilus chilensis | 46.9 | SEQ ID NO: 85 |
| Geodermatophilus sp. TF02- | 47.7 | SEQ ID NO: 86 |
| 6 | | |
| Nocardioides sp. | 40.7 | SEQ ID NO: 87 |
| TRM66260-LWL | | |
| Aspergillus pseudotamarii | 4.4 | SEQ ID NO: 88 |
| Ilyonectria sp. MPI-CAGE- | 6.7 | SEQ ID NO: 89 |
| AT-0026 | | |
| Ophiocordyceps | 17.4 | SEQ ID NO: 90 |
| camponotirufipedis | | |
| Penicillium sp. RFL-2021a | 7.8 | SEQ ID NO: 91 |
| Ramaria rubella | 25.5 | SEQ ID NO: 92 |
| Aspergillus alliaceus | 9.8 | SEQ ID NO: 93 |
| Peniophora sp. CBMAI | 32.4 | SEQ ID NO: 94 |
| 1063 | | |
| Rhodotorula sp. CCFEE | 19.7 | SEQ ID NO: 95 |
| 5036 | | |
| Acaromyces ingoldii | 8.5 | SEQ ID NO: 96 |
| Acremonium chrysogenum | 32.5 | SEQ ID NO: 97 |
| ATCC 11550 | | |
| Sporormia fimetaria CBS | 32.9 | SEQ ID NO: 98 |
| 119925 | | |
| Tilletiopsis washingtonensis | 13.4 | SEQ ID NO: 99 |
| Diaporthe batatas | 13.0 | SEQ ID NO: 100 |
| Taphrina deformans PYCC | 7.3 | SEQ ID NO: 101 |
| 5710 | | |
| Stereum hirsutum FP-91666 | 4.5 | SEQ ID NO: 102 |
| SS1 | | |
| Pseudocercospora fijiensis | 23.7 | SEQ ID NO: 103 |
| CIRAD86 | | |
| Meira miltonrushii | 16.1 | SEQ ID NO: 104 |
| Kwoniella heveanensis CBS | 8.7 | SEQ ID NO: 105 |
| 569 | | |
| Epichloe festucae Fl1 | 3.9 | SEQ ID NO: 106 |
| Pseudomicrostroma | 11.8 | SEQ ID NO: 107 |
| glucosiphilum | | |
| Fusarium tricinctum | 35.3 | SEQ ID NO: 108 |
| Fusarium bulbicola | 6.7 | SEQ ID NO: 109 |
| Fusarium fujikuroi | 32.9 | SEQ ID NO: 110 |
| Clavulina sp. PMI_390 | 5.5 | SEQ ID NO: 111 |
| Neohortaea acidophila | 26.9 | SEQ ID NO: 112 |
| Peltaster fructicola | 27.1 | SEQ ID NO: 113 |
| Lizonia empirigonia | 30.6 | SEQ ID NO: 114 |
| Cercospora beticola | 18.7 | SEQ ID NO: 115 |
| Fusarium sporotrichioides | 12.9 | SEQ ID NO: 116 |
| Ramularia collo-cygni | 24.8 | SEQ ID NO: 117 |
| Zymoseptoria tritici | 23.9 | SEQ ID NO: 118 |
| ST99CH_3D1 | | |
| Zymoseptoria tritici | 25.1 | SEQ ID NO: 119 |
| ST99CH_1A5 | | |
| Zymoseptoria tritici | 27.9 | SEQ ID NO: 120 |
| (Speckled leaf blotch | | |
| fungus) (Septoria tritici) | | |
| Zymoseptoria tritici | 31.5 | SEQ ID NO: 121 |
| (Speckled leaf blotch | | |
| fungus) (Septoria tritici) | | |
| Zymoseptoria tritici | 32.4 | SEQ ID NO: 122 |
| (Speckled leaf blotch | | |
| fungus) (Septoria tritici) | | |
| Zymoseptoria tritici | 36.1 | SEQ ID NO: 123 |
| (Speckled leaf blotch | | |
| fungus) (Septoria tritici) | | |
| Zymoseptoria brevis | 7.3 | SEQ ID NO: 124 |
| Fusarium solani | 8.4 | SEQ ID NO: 125 |
| Polychaeton citri CBS | 6.1 | SEQ ID NO: 126 |
| 116435 | | |
| Aureobasidium subglaciale | 7.5 | SEQ ID NO: 127 |
| EXF-2481 | | |
| Ceraceosorus bombacis | 18.0 | SEQ ID NO: 128 |
| Zasmidium cellare ATCC | 21.2 | SEQ ID NO: 129 |
| 36951 | | |
| Rachicladosporium sp. | 10.9 | SEQ ID NO: 130 |
| CCFEE 5018 | | |
| Cercospora berteroae | 23.8 | SEQ ID NO: 131 |
| Baudoinia panamericana | 23.3 | SEQ ID NO: 132 |
| UAMH 10762 | | |
| Acidomyces sp. | 13.2 | SEQ ID NO: 133 |
| 'richmondensis' | | |
| Teratosphaeria nubilosa | 11.3 | SEQ ID NO: 134 |
| Pseudocercospora fuligena | 27.8 | SEQ ID NO: 135 |
| Quercus suber (Cork oak) | 8.1 | SEQ ID NO: 136 |
| Elsinoe fawcettii | 28.1 | SEQ ID NO: 137 |
| Sphaerulina musiva SO2202 | 21.5 | SEQ ID NO: 138 |
| Filobasidium floriforme | 8.5 | SEQ ID NO: 139 |
| Mixia osmundae IAM 14324 | 25.5 | SEQ ID NO: 140 |
| Violaceomyces palustris | 23.2 | SEQ ID NO: 141 |
| Testicularia cyperi | 4.8 | SEQ ID NO: 142 |
| Friedmanniomyces | 4.3 | SEQ ID NO: 143 |
| endolithicus | | |
| Pseudozyma flocculosa PF-1 | 28.4 | SEQ ID NO: 144 |
| Moesziomyces antarcticus | 28.9 | SEQ ID NO: 145 |
| Sporisorium scitamineum | 5.9 | SEQ ID NO: 146 |
| Ilumatobacter sp. | 47.7 | SEQ ID NO: 147 |
| Balneolaceae bacterium | 7.5 | SEQ ID NO: 148 |

### Example 10: Generation of a Base Strain for Phytoene Desaturase Screening

To convert the farnesene base strain described in Example 5 to have high flux to the C-20 isoprenoid retinol, 2 copies of a geranylgeranylpyrophosphate synthase (GGPPS) were integrated into the genome, followed by two copies of the gene encoding a bi-functional enzyme (CrtYB) with phytoene synthase and lycopene cyclase activity, one copy of Beta-carotene 15-15′-dioxygenases (BCDO) and two copies of retinol dehydrogenase (RDH8) (Table 9). At this point the six copies of farnesene synthase were removed from the strain. The strain containing all genes described in Table 9 primarily produced phytoene and was capable of producing retinol in the presence of an active desaturase.

To measure the activity of phytoene desaturases in vivo in S. cerevisiae, a landing pad was introduced into this screening strain, which allowed for the rapid insertion of desaturase variants. The landing pad consists of 500 bp of locus-targeting DNA sequences on either end of the construct to the genomic region downstream of the yeast locus of choice (Upstream locus and Downstream locus), thereby integrating the new sequence in the chromosome of the base strain. Internally, the landing pad contained a promoter which could be GAL1, GAL3 or any other promoter of yeast GAL regulon, and a yeast native terminator of choice flanking an endonuclease recognition site. DNA variants of the phytoene desaturase library were used to transform the strain along with a plasmid expressing endonuclease, which created a double strand break at the recognition sequence and facilitated homologous recombination of the DNA variants at the site. At least six colonies from each transformation were used to screen for desaturase activity, using methods described in Example 2 and Example 3.

TABLE 9
List of Genes, and Copy Number Used to Convert FPP into
Base Strains to Screen for Phytoene Desaturase Variants

| Enzyme | Copy | |
|---|---|---|
| Name | Number | SEQ ID NO: |
| Bt. GGPPS | 2 | SEQ ID NO: 9 |
| Xd. CrtYB | 2 | SEQ ID NO: 10 |
| Pb. BCDO | 1 | SEQ ID NO: 61 |
| Dr. RDH8 | 2 | SEQ ID NO: 51 |

### Example 11: Identification of Proteins with Phytoene Desaturase Activity

Phytoene desaturase enzymes catalyze the conversion of phytoene to lycopene. Native enzymes from four different fungal species demonstrated activity in converting phytoene into lycopene when expressed in S. cerevisiae, phytoene producing strain. This library of genes was then screened in an engineered S. cerevisiae strain described in Example 10. The immediate product of the phytoene desaturase is lycopene, but retinol was used as a primary readout for desaturase activity, as a functional desaturase increases lycopene production; downstream enzymes CrtYB, RDH8 and BCDO were not limiting in this screening strain.

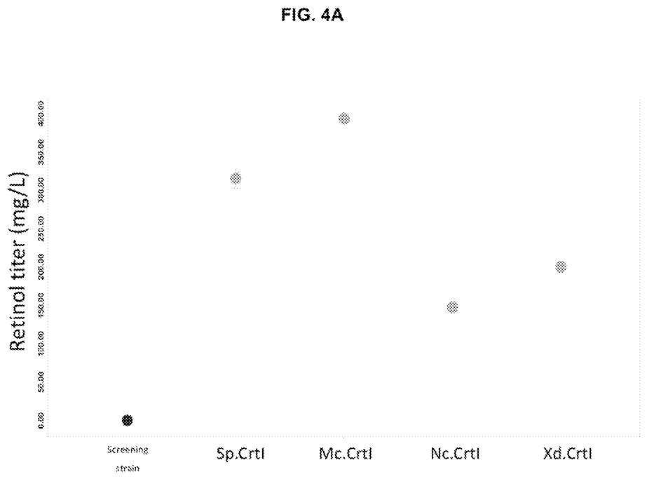

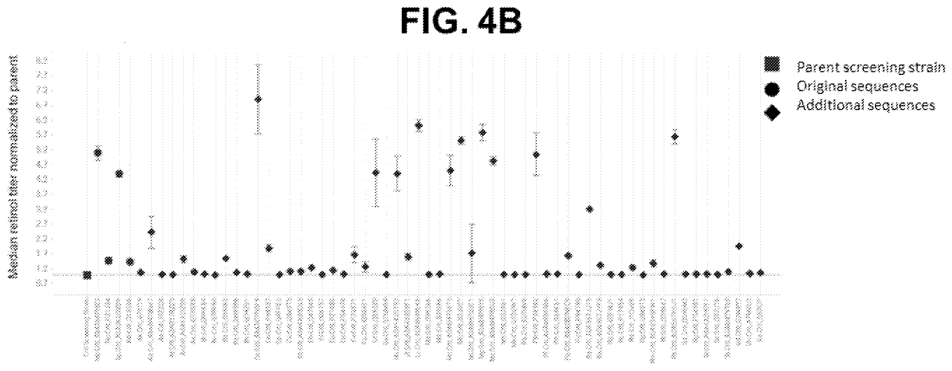

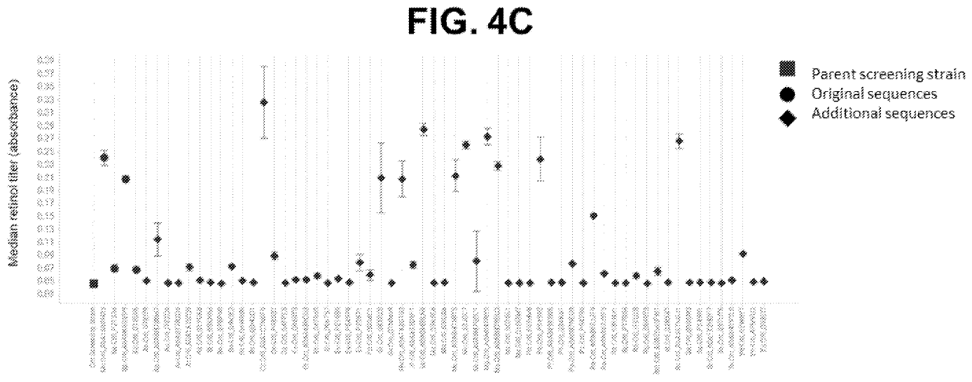

Strains expressing each of the phytoene desaturase enzymes (Xd. CrtI (SEQ ID NO: 11)); Mc.CrtI (SEQ ID NO: 150); Sp.CrtI (SEQ ID NO: 151); and Nc.CrtI (SEQ ID NO: 152)) produced retinol at least one standard deviation higher than the screening strain and were classified as hits (FIG. 4A). A further screen was performed with additional phytoene desaturase enzymes (SEQ ID NOs: 158-216) as shown in FIGS. 4B and 4C (SEQ ID NOs (in order of appearance): SEQ ID NO: 150, SEQ ID NO: 152, SEQ ID NO: 151, SEQ ID NO: 11, SEQ ID NOs: 158-216, non-inclusive of parent strain at left of chart). The parent strain for analysis of the further screening was Y89020 and 8 replicates were performed per strain.

### Example 12: Generation of a Base Strain for Screening of Bi-Functional Enzymes with Phytoene Synthase and Lycopene Cyclase Activity

To convert the farnesene base strain described in Example 5 to have high flux to the C-20 isoprenoid retinal, 2 copies of a geranylgeranylpyrophosphate synthase (GGPPS) (SEQ ID NO: 9) were integrated into the genome, followed by two copies of the gene encoding a phytoene desaturase (CrtI) (SEQ ID NO: 11), and one copy of beta-carotene 15-15′-dioxygenases (BCDO) (Table 10). At this point the six copies of farnesene synthase were removed from the strain. The strain containing all genes described in Table 10 primarily produced GGPP and was capable of producing retinal in the presence of active phytoene synthases and lycopene cyclases.

To measure the activity of bi-functional phytoene synthase/lycopene cyclases in vivo in S. cerevisiae, a landing pad was introduced into this screening strain, which allowed for the rapid insertion of gene candidates. The landing pad consisted of 500 bp of locus-targeting DNA sequences on either end of the construct to the genomic region downstream of the yeast locus of choice (Upstream locus and Downstream locus), thereby integrating the new sequence in the chromosome of the base strain. Internally, the landing pad contained a promoter which could be GAL1, GAL3 or any other promoter of yeast GAL regulon, and a yeast native terminator of choice flanking an endonuclease recognition site. DNA variants of the bi-functional phytoene synthase/lycopene cyclase library were used to transform the strain along with a plasmid expressing endonuclease, which created a double strand break at the recognition sequence and facilitated homologous recombination of the DNA variants at the site. At least six colonies from each transformation were used to screen for phytoene synthase/lycopene cyclase activity, using methods described in Example 2 and Example 3.

TABLE 10
List of Genes, and Copy Number Used to Convert FPP
into Base Strains to Screen for Bi-Functional Phytoene
Synthase and Lycopene Cyclase Variants

| Enzyme | Copy | |
|---|---|---|
| Name | Number | SEQ ID NO: |
| Bt. GGPPS | 2 | SEQ ID NO: 9 |
| Xd. CrtI | 2 | SEQ ID NO: 11 |
| Pb. BCDO | 1 | SEQ ID NO: 61 |

### Example 13: Identification of Novel Bi-Functional Enzymes with Phytoene Synthase and Lycopene Cyclase Activity

Bi-functional enzymes with phytoene synthase and lycopene cyclase activity catalyze two enzymatic steps, the conversion of GGPP to phytoene and the conversion of lycopene to beta-carotene. Bi-functional phytoene synthase and lycopene cyclase enzymes from five different fungal species demonstrated activity in producing retinal when expressed in an engineered S. cerevisiae strain described in Example 12.

The immediate product of bi-functional phytoene synthases/lycopene cyclases is phytoene but in the presence of active lycopene desaturases, the main product is beta-carotene. In this screening, retinal was used as a primary readout for phytoene synthase/lycopene cyclase activity; CrtI and BCDO enzymes were not limiting in this screening strain.

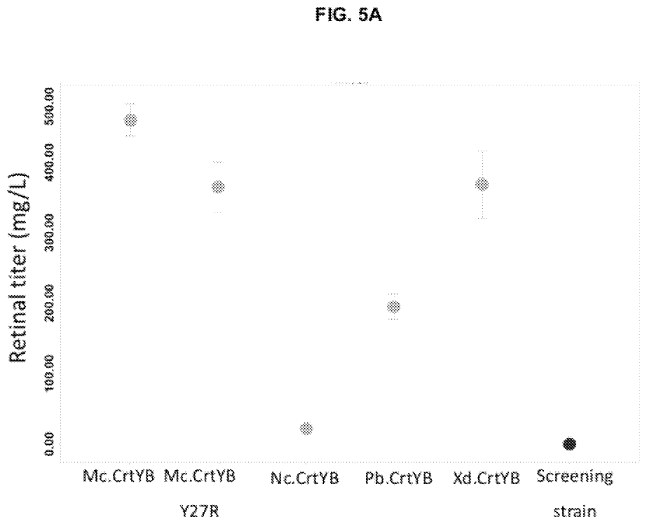

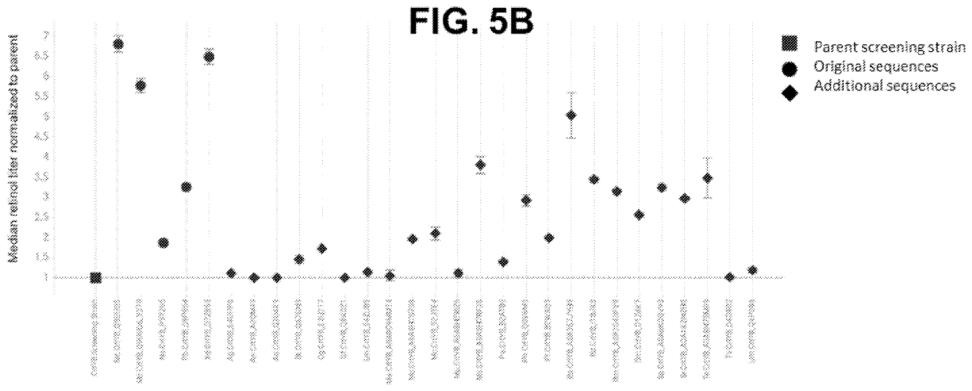

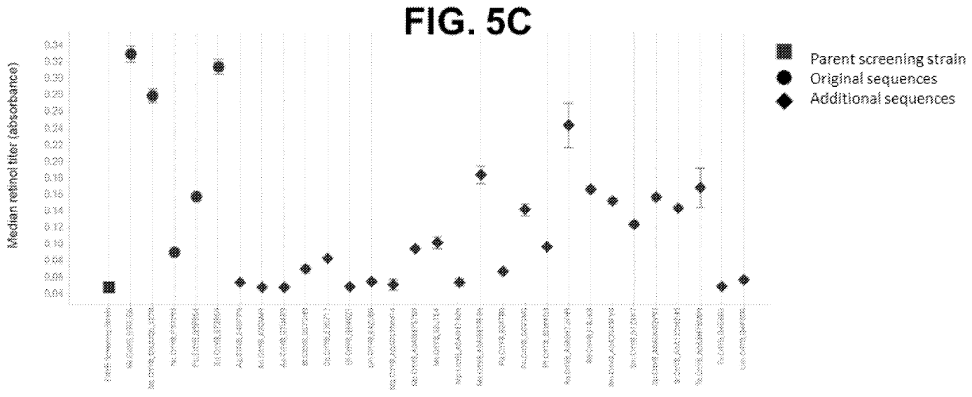

Strains expressing each of the bi-functional phytoene synthases/lycopene cyclases (Xd.CrtYB (SEQ ID NO: 10); Mc.CrtYB (SEQ ID NO: 153); Mc.CrtYB Y27R variant (SEQ ID NO: 154); Pb.CrtYB (SEQ ID NO: 155); and Nc.CrtYB (SEQ ID NO: 156)) produced retinal at least one standard deviation higher than the screening strain and were classified as hits (FIG. 5A). A further screen was performed with additional phytoene desaturase enzymes (SEQ ID NOs: 158-216) as shown in FIGS. 5B and 5C (SEQ ID NOS (in order of appearance): SEQ ID NO: 153, SEQ ID NO: 154, SEQ ID NO: 156, SEQ ID NO: 155, SEQ ID NO: 10, SEQ ID NOs: 217-240, non-inclusive of parent strain at left of chart). The parent strain for analysis of the further screening was Y89019 and 8 replicates were performed per strain.

### Example 14: Generation of a Base Strain for Screening of Monofunctional Enzymes with Lycopene Cyclase Activity

To convert the farnesene base strain described in Example 5 to have high flux to the beta-carotene, 2 copies of a geranylgeranylpyrophosphate synthase (GGPPS) (SEQ ID NO: 9) were integrated into the genome, followed by two copies of the gene encoding a phytoene desaturase (CrtI) (SEQ ID NO: 11) and two copies of phytoene synthases (Xd.CrtYB_D52G, Xd.CrtYB_E83K (Table 11). The phytoene synthases used for this screening are mutated versions of the bifunctional phytoene synthase/lycopene cyclase enzyme Xd.CrtYB. The mutant enzymes contain a mutation in the lycopene cyclase active site to eliminate cyclase activity (Xie, Wenping, et al. “Construction of lycopene-overproducing Saccharomyces cerevisiae by combining directed evolution and metabolic engineering.” Metabolic engineering 30 (2015): 69-78). The strain containing all genes described in Table 11 primarily produced lycopene and was capable of producing beta-carotene in the presence of active lycopene cyclases.

To measure the activity of mono-functional lycopene cyclases in vivo in S. cerevisiae, a landing pad was introduced into this screening strain, which allowed for the rapid insertion of two copies of each gene candidates. The landing pad consisted of 500 bp of locus-targeting DNA sequences on either end of the construct to the genomic region downstream of the yeast locus of choice (Upstream locus and Downstream locus), thereby integrating the new sequence in the chromosome of the base strain. Internally, the landing pad contained a promoter which could be GAL1, GAL3 or any other promoter of yeast GAL regulon, and a yeast native terminator of choice flanking an endonuclease recognition site. DNA variants of the mono-functional lycopene cyclase library were used to transform the strain along with a plasmid expressing endonuclease, which created a double strand break at the recognition sequence and facilitated homologous recombination of the DNA variants at the site. At least six colonies from each transformation were used to screen for lycopene cyclase activity, using methods described in Example 16.

TABLE 11
List of Genes, and Copy Number Used to Convert FPP into Base Strains
to Screen for mono-Functional Lycopene Cyclase Variants

| Enzyme | Copy | |
|---|---|---|
| Name | Number | SEQ ID NO: |
| Bt. GGPPS | 2 | SEQ ID NO: 9 |
| Xd. CrtI | 4 | SEQ ID NO: 11 |
| Xd. CrtYB | 1 | SEQ ID NO: 241 |
| mutant D52G | | |
| Xd. CrtYB | 1 | SEQ ID NO: 242 |
| mutant E83K | | |

### Example 15: Identification of Monofunctional Enzymes with Lycopene Cyclase Activity

Lycopene cyclases can be found in nature as bi-functional enzymes fused to phytoene synthase domains (Example 13) and can also be found as monofunctional enzymes. The monofunctional enzymes catalyze the conversion of lycopene to beta-carotene. In this screening, beta-carotene was used as a primary readout for lycopene cyclase activity; GGPP and CrtI enzymes were not limiting in this screening strain.

A query sequence for lycopene cyclase from Erwinia uredovora chosen based on literature reports (Eu.CrtY, P54974) and used to perform basic local alignment search tool queries against the Universal Protein resource database UniProt (Nucleic Acids Research, Volume 51, Issue D1, 6 Jan. 2023, Pages D523-D531, https://doi.org/10.1093/nar/gkac1052) as well as GenBank (Nucleic Acids Research, 2013 January; 41 (D1): D36-42). Ultimately, 46 protein sequences were codon-optimized for S. cerevisiae and ordered from a DNA-synthesis vendor (SEQ ID NOs: 243-288). Hits were determined by calculating the percent conversion of measured lycopene and phytoene to beta-carotene.

$$Percent conversion ( \% ) = \frac{β - carotene ( mmol / L ) \times 100}{β - c a r otene ( mmol / L ) + lycopene ( mmol / L ) + phytoene ( mmol / L )}$$

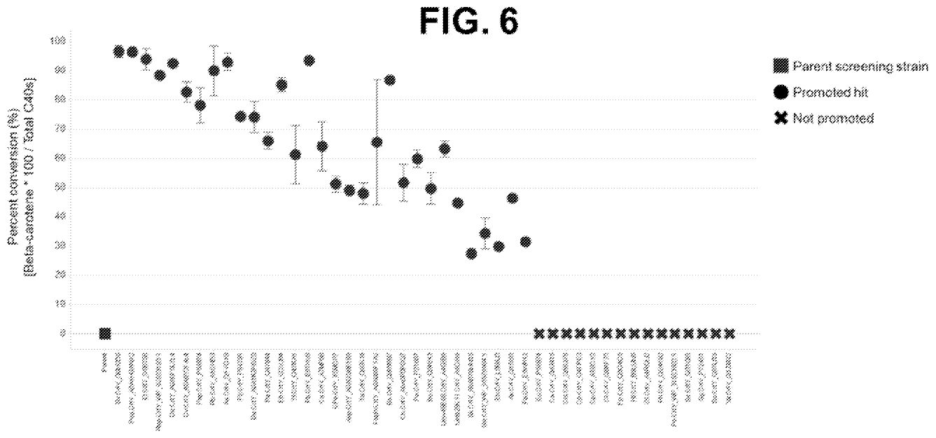

Out of 46 enzymes in the natural biodiversity library screened, 31 produced beta-carotene and were classified as hits (FIG. 6; in order of appearance SEQ ID NOs: 243-288 (hits: SEQ ID NOs: 243-273) non-inclusive of parent strain at left). The screening strain parent only made phytoene and lycopene.

The percentage conversion of lycopene and phytoene to beta-carotene for the 31 hits ranged from 30-99%. The screening strain parent does not make any beta-carotene.

### Example 16: Analytical Methods for Product Extraction and Quantification of Carotenoids

After incubation of the production plate, plates were centrifuged (5 min, 4250 rpm, 20° C.) to pellet cell biomass and the supernatant was discarded. To each well, 600 μL of dimethyl sulfoxide (DMSO) was added. The plate was then sealed with aluminum and shaken for 30 minutes at 1500 rpm. The seal was then removed, 600 μL of heptane added to each well, resealed, and then shaken again for 30 minutes at 1500 rpm. The seal was then removed a 3^{rd} time, 50 μL of phosphate-buffered saline (PBS) added, resealed, and shaken for 5 minutes at 1500 rpm. The layers were separated through centrifugation of the plate (5 min, 4250 rpm, 20° C.) and 200 μL of the heptane layer was then transferred to an empty 1.1 ml 96-well plate and sealed. The sample plate was then stored at −20° C. until analysis.

Sample plates were analyzed for beta-carotene titer on a weight by volume basis through external calibration with authentic standards. Measurements for phytoene and lycopene were given based on relative response factors to beta-carotene. Analyses of samples was performed using ultra-high performance liquid chromatography and ultraviolet detection (UPLC-UV) on a Thermo Fisher Vanquish system equipped with a variable wavelength detector.

TABLE 12
Mobile Phases and Column Information

| | |
|---|---|
| Mobile | 50% MilliQ water 50% methanol, 0.1% acetic acid, 5 mM |
| Phase A | ammonium acetate |
| Mobile | 1:8:1 methanol:isopropyl alcohol:MilliQ water, 0.1% |
| Phase B | acetic acid, 5 mM ammonium acetate |
| Needle wash | 100% isopropyl alcohol |
| Rear Seal | 75% isopropyl alcohol/ 25% water with 0.1% formic acid |
| Wash | |
| Column | Agilent Zorbax Eclipse Plus C8 1.8 um 100 mm × 2.1 |
| | mm P/N 959758-906 |

TABLE 13
Mobile Phase Gradient

| Time | Flow (mL/min) | % A | % B | Curve |
|---|---|---|---|---|
| 0 | 0.4 | 98 | 2 | 5 |
| 0.5 | 0.4 | 98 | 2 | 5 |
| 1.0 | 0.5 | 65 | 35 | 5 |
| 1.5 | 0.5 | 50 | 50 | 5 |
| 2.0 | 0.5 | 40 | 60 | 5 |
| 5.0 | 0.5 | 10 | 90 | 5 |
| 8.2 | 0.5 | 5 | 95 | 5 |
| 8.4 | 0.5 | 98 | 2 | 5 |
| 9.0 | 0.4 | 98 | 2 | 5 |

TABLE 14
Column Compartment Settings

| | |
|---|---|
| Temperature Control: | On |
| Temperature: | 45° C. |
| Temperature Range: | Within ± 0.5° C. |
| Equilibration Time: | 0 min |

TABLE 15
Multisampler settings

| | |
|---|---|
| Needle Wash | Standard Wash |
| Draw Speed | 5 μL/sec |
| Dispense Speed | 5 μL/sec |
| Wash mode | Both and after draw |
| Wash time | 10 sec |
| Wash speed | 50 μL/sec |
| Cooler | On, 4° C. |

TABLE 16
Detector Settings

| | |
|---|---|
| Wavelength | 250, 417, 454 nm |
| Peakwidth | 1 min (10 s response time) *0.5 Hz) |
| Lamps on required for | Variable wavelength detector or Diode array |
| acquisition | detector |

### Example 17: Base Strain for Screening of Geranylgeranyl Diphosphate Synthases (GGPPS)

To the farnesene base strain described in Example 5 was converted into a geranylgeranyl diphosphate synthase (GGPPS) screening strain that produced retinol as a readout of enzyme activity. The farnesene base strain contained two copies of ERG20, the native S. cerevisiae genomic copy, and a second copy expressed under inducible Gal promoter. The inducible copy was removed and the screening strain only expresses the native ERG20 gene to reduce flux to FPP and increase substrate pools of DMAPP and IPP. Heterologous genes to convert GGPP to retinol were added as shown on Table 17:2 copies of bifunctional phytoene synthase/lycopene cyclase enzyme Xd.CrtYB, 2 copies of phytoene desaturase Mc.CrtI, 2 copies of beta-carotene dioxygenase Pb.BCDO, and 2 copies of retinol dehydrogenase Dr.RDH8. The strain containing all genes described in Table 17 primarily produced IPP, DMAPP, FPP and was capable of producing retinol in the presence of active GGPPS.

To measure the activity of geranylgeranyl diphosphate synthases in vivo in S. cerevisiae, a landing pad was introduced into this screening strain, which allowed for the rapid insertion of one copy of each gene candidate. The landing pad consisted of 500 bp of locus-targeting DNA sequences on either end of the construct to the genomic region downstream of the yeast locus of choice (Upstream locus and Downstream locus), thereby integrating the new sequence in the chromosome of the base strain. Internally, the landing pad contained a promoter which could be GAL1, GAL3 or any other promoter of yeast GAL regulon, and a yeast native terminator of choice flanking an endonuclease recognition site. DNA variants of the GGPPS library were used to transform the strain along with a plasmid expressing endonuclease, which created a double strand break at the recognition sequence and facilitated homologous recombination of the DNA variants at the site. At least six colonies from each transformation were used to screen for GGPPS activity, using methods described in Example 16.

TABLE 17
List of Genes, and Copy Number Used to Convert FPP into Base Strains
to Screen for Geranylgeranyl diphosphate Synthase Variants

| Enzyme | Copy | |
|---|---|---|
| Name | Number | SEQ ID NO: |
| Xd. CrtYB | 2 | SEQ ID NO: 10 |
| Mc. CrtI | 2 | SEQ ID NO: 150 |
| Pb. BCDO | 2 | SEQ ID NO: 61 |
| Dr. RDH8 | 2 | SEQ ID NO: 51 |

### Example 18: Identification of New Geranylgeranyl Disphosphate Synthases (GGPP Synthases)

Geranylgeranyl diphosphate synthases (GGPP) catalyze the formation of GGPP (C20) by either condensing one molecule of farnesyl pyrophosphate (FPP, C15) with isopentenyl pyrophosphate (IPP, C5), or through three consecutive condensation of IPP “extender units” directly onto a molecule of dimethylallyl diphosphate (DMAPP, C5). Different variants of GGPPS may do one or both types of reactions to form GGPP. In this screening, retinol was used as a primary readout for GGPP synthase activity; enzymes required to convert GGPP to retinol Table 17 were not limiting in this screening strain.

Query sequences for GGPPS from Cistus criticus (Ccr2, AF492023) were used to perform basic local alignment search tool queries against the Universal Protein resource database UniProt (Nucleic Acids Research, Volume 51, Issue D1, 6 Jan. 2023, Pages D523-D531, https://doi.org/10.1093/nar/gkac1052) as well as GenBank (Nucleic Acids Research, 2013 January; 41 (D1): D36-42). Ultimately, 39 protein sequences were codon-optimized for S. cerevisiae and ordered from a DNA-synthesis vendor (SEQ ID NOs: 289-327). Hits were determined by measuring retinol titers. For enzymes of plant origin, N-terminal truncations were performed through alignment to remove possible signal sequences.

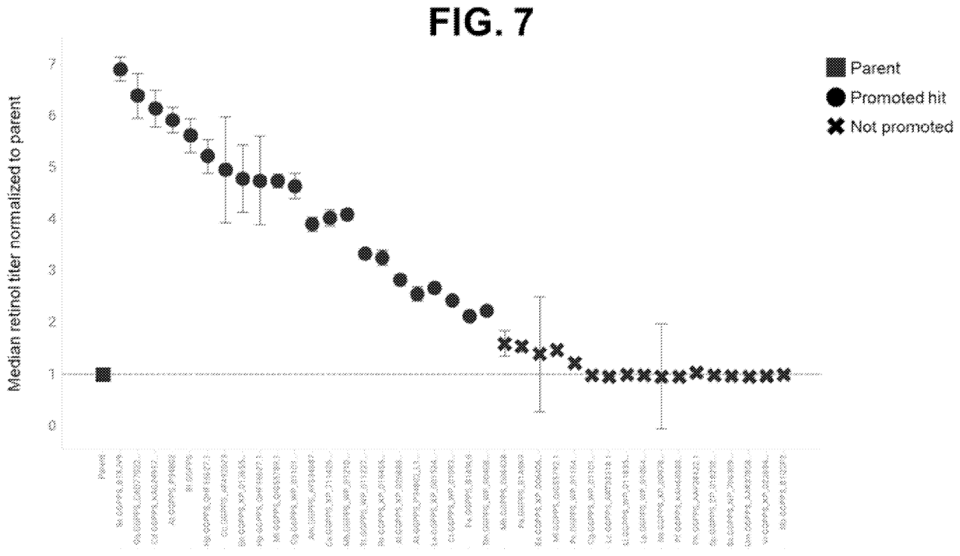

Out of 39 enzymes in the natural biodiversity library screened, 22 (SEQ ID NOs: 289-310) had retinol titers at least 2-fold higher than the parent and were classified as hits (FIG. 7; SEQ ID NOs: 289-327 in order of appearance, non-inclusive of parent strain at left). The screening strain parent does not make any retinol. The enzymes classified as hits had retinol titers ranging from 2.1×-6.9× that of the parent.

One or more features from any embodiments described herein or in the figures may be combined with one or more features of any other embodiment described herein in the figures without departing from the scope of the invention.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

SEQUENCE LISTING

SEQ ID NO: 1 (Acetyl-CoA acetyltransferase P41338)
MSQNVYIVSTARTPIGSFQGSLSSKTAVELGAVALKGALAKVPELDASKDFDEIIFGN
VLSANLGQAPARQVALAAGLSNHIVASTVNKVCASAMKAIILGAQSIKCGNADVVV
AGGCESMTNAPYYMPAARAGAKFGQTVLVDGVERDGLNDAYDGLAMGVHAEKC
ARDWDITREQQDNFAIESYQKSQKSQKEGKFDNEIVPVTIKGFRGKPDTQVTKDEEP
ARLHVEKLRSARTVFQKENGTVTAANASPINDGAAAVILVSEKVLKEKNLKPLAIIK
GWGEAAHQPADFTWAPSLAVPKALKHAGIEDINSVDYFEFNEAFSVVGLVNTKILKL
DPSKVNVYGGAVALGHPLGCSGARVVVTLLSILQQEGGKIGVAAICNGGGGASSIVI
EKI
SEQ ID NO: 2 (Hydroxymethylglutaryl-CoA synthase P54839)
MKLSTKLCWCGIKGRLRPQKQQQLHNTNLQMTELKKQKTAEQKTRPQNVGIKGIQI
YIPTQCVNQSELEKFDGVSQGKYTIGLGQTNMSFVNDREDIYSMSLTVLSKLIKSYNI
DTNKIGRLEVGTETLIDKSKSVKSVLMQLFGENTDVEGIDTLNACYGGTNALFNSLN
WIESNAWDGRDAIVVCGDIAIYDKGAARPTGGAGTVAMWIGPDAPIVFDSVRASYM
EHAYDFYKPDFTSEYPYVDGHFSLTCYVKALDQVYKSYSKKAISKGLVSDPAGSDA
LNVLKYFDYNVFHVPTCKLVTKSYGRLLYNDFRANPQLFPEVDAELATRDYDESLT
DKNIEKTFVNVAKPFHKERVAQSLIVPTNTGNMYTASVYAAFASLLNYVGSDDLQG
KRVGLFSYGSGLAASLYSCKIVGDVQHIIKELDITNKLAKRITETPKDYEAAIELRENA
HLKKNFKPQGSIEHLQSGVYYLTNIDDKFRRSYDVKK
SEQ ID NO: 3 (3-hydroxy-3-methylglutaryl-coenzyme A reductase 2 P12684)
MSLPLKTIVHLVKPFACTARFSARYPIHVIVVAVLLSAAAYLSVTQSYLNEWKLDSN
QYSTYLSIKPDELFEKCTHYYRSPVSDTWKLLSSKEAADIYTPFHYYLSTISFQSKDNS
TTLPSLDDVIYSVDHTRYLLSEEPKIPTELVSENGTKWRLRNNSNFILDLHNIYRNMV
KQFSNKTSEFDQFDLFIILAAYLTLFYTLCCLFNDMRKIGSKFWLSFSALSNSACALYL
SLYTTHSLLKKPASLLSLVIGLPFIVVIIGFKHKVRLAAFSLQKFHRISIDKKITVSNIIYE
AMFQEGAYLIRDYLFYISSFIGCAIYARHLPGLVNFCILSTFMLVFDLLLSATFYSAILS
MKLEINIIHRSTVIRQTLEEDGVVPTTADIIYKDETASEPHFLRSNVAIILGKASVIGLLL
LINLYVFTDKLNATILNTVYFDSTIYSLPNFINYKDIGNLSNQVIISVLPKQYYTPLKKY
HQIEDSVLLIIDSVSNAIRDQFISKLLFFAFAVSISINVYLLNAAKIHTGYMNFQPQSNK
IDDLVVQQKSATIEFSETRSMPASSGLETPVTAKDIIISEEIQNNECVYALSSQDEPIRPL
SNLVELMEKEQLKNMNNTEVSNLVVNGKLPLYSLEKKLEDTTRAVLVRRKALSTLA
ESPILVSEKLPFRNYDYDRVFGACCENVIGYMPIPVGVIGPLIIDGTSYHIPMATTEGC
LVASAMRGCKAINAGGGATTVLTKDGMTRGPVVRFPTLIRSGACKIWLDSEEGQNSI
KKAFNSTSRFARLQHIQTCLAGDLLFMRFRITTGDAMGMNMISKGVEYSLKQMVEE
YGWEDMEVVSVSGNYCTDKKPAAINWIEGRGKSVVAEATIPGDVVKSVLKSDVSAL
VELNISKNLVGSAMAGSVGGFNAHAANLVTALFLALGQDPAQNVESSNCITLMKEV
DGDLRISVSMPSIEVGTIGGGTVLEPQGAMLDLLGVRGPHPTEPGANARQLARIIACA
VLAGELSLCSALAAGHLVQSHMTHNRKTNKANELPQPSNKGPPCKTSALL
SEQ ID NO: 4 (3-hydroxy-3-methylglutaryl-coenzyme A reductase 1 P12683)
MPPLFKGLKQMAKPIAYVSRFSAKRPIHIILFSLIISAFAYLSVIQYYFNGWQLDSNSVF
ETAPNKDSNTLFQECSHYYRDSSLDGWVSITAHEASELPAPHHYYLLNLNFNSPNET
DSIPELANTVFEKDNTKYILQEDLSVSKEISSTDGTKWRLRSDRKSLFDVKTLAYSLY
DVFSENVTQADPFDVLIMVTAYLMMFYTIFGLFNDMRKTGSNFWLSASTVVNSASS
LFLALYVTQCILGKEVSALTLFEGLPFIVVVVGFKHKIKIAQYALEKFERVGLSKRITT
DEIVFESVSEEGGRLIQDHLLCIFAFIGCSMYAHQLKTLTNFCILSAFILIFELILTPTFYS
AILALRLEMNVIHRSTIIKQTLEEDGVVPSTARIISKAEKKSVSSFLNLSVVVIIMKLSVI
LLFVFINFYNFGANWVNDAFNSLYFDKERVSLPDFITSNASENFKEQAIVSVTPLLYY
KPIKSYQRIEDMVLLLLRNVSVAIRDRFVSKLVLSALVCSAVINVYLLNAARIHTSYT
ADQLVKTEVTKKSFTAPVQKASTPVLINKTVISGSKVKSLSSAQSSSSGPSSSSEEDDS
RDIESLDKKIRPLEELEALLSSGNTKQLKNKEVAALVIHGKLPLYALEKKLGDTTRAV
AVRRKALSILAEAPVLASDRLPYKNYDYDRVFGACCENVIGYMPLPVGVIGPLVIDG
TSYHIPMATTEGCLVASAMRGCKAINAGGGATTVLTKDGMTRGPVVRFPTLKRSGA
CKIWLDSEEGQNAIKKAFNSTSRFARLQHIQTCLAGDLLFMRFRTTTGDAMGMNMIS
KGVEYSLKQMVEEYGWEDMEVVSVSGNYCTDKKPAAINWIEGRGKSVVAEATIPG
DVVRKVLKSDVSALVELNIAKNLVGSAMAGSVGGFNAHAANLVTAVFLALGQDPA
QNVESSNCITLMKEVDGDLRISVSMPSIEVGTIGGGTVLEPQGAMLDLLGVRGPHAT
APGTNARQLARIVACAVLAGELSLCAALAAGHLVQSHMTHNRKPAEPTKPNNLDAT
DINRLKDGSVTCIKS
SEQ ID NO: 5 (farnesyl pyrophosphate synthase)
MASEKEIRRERFLNVFPKLVEELNASLLAYGMPKEACDWYAHSLNYNTPGGKLNRG
LSVVDTYAILSNKTVEQLGQEEYEKVAILGWCIELLQAYFLVADDMMDKSITRRGQP
CWYKVPEVGEIAINDAFMLEAAIYKLLKSHFRNEKYYIDITELFHEVTFQTELGQLMD
LITAPEDKVDLSKFSLKKHSFIVTFKTAYYSFYLPVALAMYVAGITDEKDLKQARDV
LIPLGEYFQIQDDYLDCFGTPEQIGKIGTDIQDNKCSWVINKALELASAEQRKTLDEN
YGKKDSVAEAKCKKIFNDLKIDQLYHEYEESVAKDLKAKISQVDESRGFKADVLTAF
LNKVYKRSK
SEQ ID NO: 6 (Phosphomevalonate kinase P24521)
MSELRAFSAPGKALLAGGYLVLDTKYEAFVVGLSARMHAVAHPYGSLQGSDKFEV
RVKSKQFKDGEWLYHISPKSGFIPVSIGGSKNPFIEKVIANVFSYFKPNMDDYCNRNL
FVIDIFSDDAYHSQEDSVTEHRGNRRLSFHSHRIEEVPKTGLGSSAGLVTVLTTALASF
FVSDLENNVDKYREVIHNLAQVAHCQAQGKIGSGFDVAAAAYGSIRYRRFPPALISN
LPDIGSATYGSKLAHLVDEEDWNITIKSNHLPSGLTLWMGDIKNGSETVKLVQKVKN
WYDSHMPESLKIYTELDHANSRFMDGLSKLDRLHETHDDYSDQIFESLERNDCTCQK
YPEITEVRDAVATIRRSFRKITKESGADIEPPVQTSLLDDCQTLKGVLTCLIPGAGGYD
AIAVITKQDVDLRAQTANDKRFSKVQWLDVTQADWGVRKEKDPETYLDK
SEQ ID NO: 7 (Diphosphomevalonate decarboxylase P32377)
MTVYTASVTAPVNIATLKYWGKRDTKLNLPTNSSISVTLSQDDLRTLTSAATAPEFE
RDTLWLNGEPHSIDNERTQNCLRDLRQLRKEMESKDASLPTLSQWKLHIVSENNFPT
AAGLASSAAGFAALVSAIAKLYQLPQSTSEISRIARKGSGSACRSLFGGYVAWEMGK
AEDGHDSMAVQIADSSDWPQMKACVLVVSDIKKDVSSTQGMQLTVATSELFKERIE
HVVPKRFEVMRKAIVEKDFATFAKETMMDSNSFHATCLDSFPPIFYMNDTSKRIISW
CHTINQFYGETIVAYTFDAGPNAVLYYLAENESKLFAFIYKLFGSVPGWDKKFTTEQ
LEAFNHQFESSNFTARELDLELQKDVARVILTQVGSGPQETNESLIDAKTGLPKE
SEQ ID NO: 8 (Isopentenyl-diphosphate Delta-isomerase P15496)
MTADNNSMPHGAVSSYAKLVQNQTPEDILEEFPEIIPLQQRPNTRSSETSNDESGETC
FSGHDEEQIKLMNENCIVLDWDDNAIGAGTKKVCHLMENIEKGLLHRAFSVFIFNEQ
GELLLQQRATEKITFPDLWTNTCCSHPLCIDDELGLKGKLDDKIKGAITAAVRKLDHE
LGIPEDETKTRGKFHFLNRIHYMAPSNEPWGEHEIDYILFYKINAKENLTVNPNVNEV
RDFKWVSPNDLKTMFADPSYKFTPWFKIICENYLFNWWEQLDDLSEVENDRQIHRM
L
SEQ ID NO: 9 (Bt.GGPPS; GGPP Synthase)
MLTSSKSIESFPKNVQPYGKHYQNGLEPVGKSQEDILLEPFHYLCSNPGKDVRTKMIE
AFNAWLKVPKDDLIVITRVIEMLHSASLLIDDVEDDSVLRRGVPAAHHIYGTPQTINC
ANYVYFLALKEIAKLNKPNMITIYTDELINLHRGQGMELFWRDTLTCPTEKEFLDMV
NDKTGGLLRLAVKLMQEASQSGTDYTGLVSKIGIHFQVRDDYMNLQSKNYADNKG
FCEDLTEGKFSFPIIHSIRSDPSNRQLLNILKQRSSSIELKQFALQLLENTNTFQYCRDFL
RVLEKEAREEIKLLGGNIMLEKIMDVLSVNE
SEQ ID NO: 10 (Xd.CrtYB; Q7Z859; Phytone synthase_beta-carotene synthase fusion;
Phaffia rhodozyma (Xanthophyllomyces dendrorhous))
MTALAYYQIHLIYTLPILGLLGLLTSPILTKFDIYKISILVFIAFSATTPWDSWIIRNGAW
TYPSAESGQGVFGTFLDVPYEEYAFFVIQTVITGLVYVLATRHLLPSLALPKTRSSALS
LALKALIPLPIIYLFTAHPSPSPDPLVTDHYFYMRALSLLITPPTMLLAALSGEYAFDW
KSGRAKSTIAAIMIPTVYLIWVDYVAVGQDSWSINDEKIVGWRLGGVLPIEEAMFFLL
TNLMIVLGLSACDHTQALYLLHGRTIYGNKKMPSSFPLITPPVLSLFFSSRPYSSQPKR
DLELAVKLLEEKSRSFFVASAGFPSEVRERLVGLYAFCRVTDDLIDSPEVSSNPHATID
MVSDFLTLLFGPPLHPSQPDKILSSPLLPPSHPSRPTGMYPLPPPPSLSPAELVQFLTER
VPVQYHFAFRLLAKLQGLIPRYPLDELLRGYTTDLIFPLSTEAVQARKTPIETTADLLD
YGLCVAGSVAELLVYVSWASAPSQVPATIEEREAVLVASREMGTALQLVNIARDIKG
DATEGRFYLPLSFFGLRDESKLAIPTDWTEPRPQDFDKLLSLSPSSTLPSSNASESFRFE
WKTYSLPLVAYAEDLAKHSYKGIDRLPTEVQAGMRAACASYLLIGREIKVVWKGDV
GERRTVAGWRRVRKVLSVVMSGWEGQ
SEQ ID NO: 11 (Xd.CrtI; Lycopene synthase; Phaffia rhodozyma (Xanthophyllomyces
dendrorhous))
MGKEQDQDKPTAIIVGCGIGGIATAARLAKEGFQVTVFEKNDYSGGRCSLIERDGYR
FDQGPSLLLLPDLFKQTFEDLGEKMEDWVDLIKCEPNYVCHFHDEETFTFSTDMALL
KREVERFEGKDGFDRFLSFIQEAHRHYELAVVHVLQKNFPGFAAFLRLQFIGQILALH
PFESIWTRVCRYFKTDRLRRVFSFAVMYMGQSPYSAPGTYSLLQYTELTEGIWYPRG
GFWQVPNTLLQIVKRNNPSAKFNFNAPVSQVLLSPAKDRATGVRLESGEEHHADVVI
VNADLVYASEHLIPDDARNKIGQLGEVKRSWWADLVGGKKLKGSCSSLSFYWSMD
RIVDGLGGHNIFLAEDFKGSFDTIFEELGLPADPSFYVNVPSRIDPSAAPEGKDAIVILV
PCGHIDASNPQDYNKLVARARKFVIQTLSAKLGLPDFEKMIVAEKVHDAPSWEKEFN
LKDGSILGLAHNFMQVLGFRPSTRHPKYDKLFFVGASTHPGTGVPIVLAGAKLTANQ
VLESFDRSPAPDPNMSLSVPYGKPLKSNGTGIDSQVQLKFMDLERWVYLLVLLIGAV
IARSVGVLAF
SEQ ID NO: 12 (Hs.BCDO; Beta-carotene 15-15′-dioxygenase)
MDIIFGRNRKEQLEPVRAKVTGKIPAWLQGTLLRNGPGMHTVGESRYNHWFDGLAL
LHSFTIRDGEVYYRSKYLRSDTYNTNIEANRIVVSEFGTMAYPDPCKNIFSKAFSYLS
HTIPDFTDNCLINIMKCGEDFYATSETNYIRKINPQTLETLEKVDYRKYVAVNLATSH
PHYDEAGNVLNMGTSIVEKGKTKYVIFKIPATVPEGKKQGKSPWKHTEVFCSIPSRSL
LSPSYYHSFGVTENYVIFLEQPFRLDILKMATAYIRRMSWASCLAFHREEKTYIHIIDQ
RTRQPVQTKFYTDAMVVFHHVNAYEEDGCIVFDVIAYEDNSLYQLFYLANLNQDFK
ENSRLTSVPTLRRFAVPLHVDKNAEVGTNLIKVASTTATALKEEDGQVYCQPEFLYE
GLELPRVNYAHNGKQYRYVFATGVQWSPIPTKIIKYDILTKSSLKWREDDCWPAEPL
FVPAPGAKDEDDGVILSAIVSTDPQKLPFLLILDAKSFTELARASVDVDMHMDLHGL
FITDMDWDTKKQAASEEQRDRASDCHGAPLT
SEQ ID NO: 13 (Sc.ADH6; Aldehyde dehydrogenase)
MSYPEKFEGIAIQSHEDWKNPKKTKYDPKPFYDHDIDIKIEACGVCGSDIHCAAGHW
GNMKMPLVVGHEIVGKVVKLGPKSNSGLKVGQRVGVGAQVFSCLECDRCKNDNEP
YCTKFVTTYSQPYEDGYVSQGGYANYVRVHEHFVVPIPENIPSHLAAPLLCGGLTVY
SPLVRNGCGPGKKVGIVGLGGIGSMGTLISKAMGAETYVISRSSRKREDAMKMGAD
HYIATLEEGDWGEKYFDTFDLIVVCASSLTDIDFNIMPKAMKVGGRIVSISIPEQHEM
LSLKPYGLKAVSISYSALGSIKELNQLLKLVSEKDIKIWVETLPVGEAGVHEAFERME
KGDVRYRFTLVGYDKEFSD
SEQ ID NO: 14 (Vanderwaltozyma polyspora) (A7TLS3)
MSYPEYFQGIGVVDKDNWTHPKHVSYAAKEFKECDVDIEIEACGVCGSDIHCASGD
WGERRDKLVVGHEVIGRVIKLGPECHTGLKIGDRVGVGAQVASCLDCERCEHDNEP
YCPKFVTTYNQAYPDGYISQGGYASHIRVHEHFVIPVPEKIPTHLAAPLLCGGITVYSP
LVRGGCAPGKKVGIVGIGGIGHMGILFAKAMGAEVYAFSRHENKREDSMKLGADH
YIATLEEEGWTEKYFNSLDVMILCASSLSQINLDDLIKVMKVEGKIISICVPENDEMMI
VKPLGLLGVYVSNSLLGSRKELLELLHMVAEKDIKIWVEKLPVGEAGVSEAFIRMKK
GDVRYRFTLTDFDKEFPKN
SEQ ID NO: 15 (Vanderwaltozyma polyspora) (A7TM83)
MAYPEMFEGIGVTNPEDWKHPKKVTYEPKKFDDQDVDIKIEACGVCGSDIHCAASH
WGPVVKNQVVGHEIVGRVVKVGPKCTTGIKIGDRVGVGAQVASCLECSRCQNDNEP
YCPRFVTTYGQPYPDGYVSQGGYASHVRVHEHFAIPVPEELPLHLAAPLLCGGITVY
SPLLRGGCGPGKKVGIVGIGGIGHMGILFAKAMGAEVYAFSRSHSKEKDAKELGAD
HYIATLEDKDWTEKYFDTLDLIVVCASSLTDVNFDELIKVMKVNTKILSISVPSIDETL
TLKPFGLLGVTIGNSALGSRKEIEQLLKLVVDKDIKIWVEQLQVGEAGVAEAFERMD
KGQVRYRFTLMGFDKEFGN
SEQ ID NO: 16 (Vanderwaltozyma polyspora) (A7TPJ1)
MSYPEKFQGIGITNREDWKHPKKVTFEPKQFNDKDVDIKIEACGVCGSDVHCAASH
WGPVAEKQVVGHEIIGRVLKVGPKCTTGIKVGDRVGVGAQAWSCLECSRCKSDNES
YCPKSVWTYSIPYIDGYVSQGGYASHIRLHEHFAIPIPDKLSNELAAPLLCGGITVYSP
LLRNGCGPGKKVGIVGIGGIGHMGLLFAKGMGAEVYAFSRTHSKEADAKKLGADHF
IATLEDKDWTTKYFDTLDLLVICASSLTDINFDELTKIMKVNTKIISISAPAADEVLTL
KPFGLIGVTIGNSAIGSRREIEHLLNFVAEKDIKPWVETLPVGEAGVNEAFERMDKGD
VKYRFTLVDFDKEFGN
SEQ ID NO: 17 (Clavispora lusitaniae) (C4Y4W4)
MAYPETFEAIGVVDYDDWKNPKRFEYKPQEFRDYDVDIEIDACGVCGSDLHAASGN
WGRPYAPVAVGHEIIGRVVKVGPKAKEGLKVGDRVGVGAQCDCDNTCAACLSKSE
PNCRNHVGTYFGHNKETGENTIGGNASHIRVNSQFVFKIPDSLKTEHAAPLLCGGITG
FAPLLQNNVGKGTRVGVVGIGGIGHMTILFAKALGAEVTAISRTDSKKEMAKKLGA
DHYAATADEDFSKYADTLDLIVNTGSSFSGSSMDKILSMLRARGKFVFITAPPATEKL
ELTPFQLLANNTSIQGSLLGTPEEIQYMLNFAAEHKIEPWVETIDMSEENLATAWKRA
EEGDVRFRFTMVNYDKCFKR
SEQ ID NO: 18 (Clavispora lusitaniae) (C4Y4W5)
MVYPETFKAVGVVDFDDWLHPKSFEYKPQEFRDYDVDIEIEACGVCGSDIHAANGD
WGRPYAPVAVGHEIIGKVVKVGPKAKEGIKVGDRVGVGAQCDCDNTCIACKKNLQ
SNCRNHVGTYFGVNKETGFKTIGGNASHIRVNSQFVFNIPDSLKTEHAAPLLCGGITG
FSPLLQHNVGKGTKVGVVGIGGIGHMTILFAKALGAEVTAISRTDSKKEVAKQLGAD
HFAATADEDFIEKYADTLDLIVNTGSSFSGSSLDKLLSMLVPRGKFVFITAPPVGENIE
LSPMKLLFNNSSIQGSAIGSPEEIQYMLNFAAEHKIEPWVETIDISEENLGKAWKRAEE
GDVKFRFTMVGYDKFFKN
SEQ ID NO: 19 (Clavispora lusitaniae) (C4YAF2)
MVYPETFQALGVVDYDDWLHPKTFEYKPQEFRDYDIDIEIEACGVCGSDIHAASGN
WGRPYAPVAVGHEIVGKVVKVGPKAKSGIELGDRVGVGAQCDCDNSCIACELNLQS
NCRNHVGTYFGHNKDTGFNTIGGNASHIRVNSQFAFKIPPSLKTEHVAPLLCGGITGF
SPLLQHKVGKGTKVGVVGIGGIGHMTILFAKALGAEVTAISRTDSKKDLAEKLGADH
YAATSDEEFEKKYSDTLDIIVNTGSSFAGSAVEKLLKMLRPRGKFVFITVPPSGENLEL
SPVQLVSNNASVQGSSTGSLEEIQFMLDFAAEHNIQPWVETIDISEENLGKAWERAQK
GDVKFRFTMVGYDKFFKKK
SEQ ID NO: 20 (Eremothecium cymbalariae) (G8JME0)
MGELKEITGIAVVEGTEWTSPKKVSFKPKTVYARDIVLKVEACGVCGSDIHCAAGH
WGPRNNPLVVGHEIIGRVIEVGSGCKEGLKVGDRVGVGAQALSCLECSRCESENEQY
CSNMTGTYNFPYPDGYISQGGYASHVIVHEHFAIPIPDNIPSHLAAPLMCGGITAFSPL
LRSNVGPGTSVGVVGIGGIGHMAVIFAKALGANVTAISRRSNKKDDALKLGADVYIA
AVEDKDWHKTYANTLDLIVVCSSSLTELDFDRIPSLLKVGGKVLSIAIPEASERLVLRP
LLLKGISIGTSVIGSPAEIKKLLDFVSKKNLKIWVEEVPISEAGVAEVFRRMNSGDVRY
RFSLVDFDKEFGA
SEQ ID NO: 21 (Kluyveromyces lactis) (Q6CMI8)
MSYPDNFEGFAVVDSHKKWLSPEKVVYPAKQFNPRDVDIEIEACGVCGSDVHCAKG
NWGEMKLPLVVGHEIIGKVVRLGDKCDTGLKVGDRVGVGAQVSACLDCNRCNND
NEQYCPKFVTTYSQPYFEDGYVGQGGYASHIRVHEHFVIPIPESLETNNVAPLLCGGA
TVYSPLKRNGCGPGKKVGIVGIGGIGHMGILLAKAMGAEVYAISRSHAKEETSKQLG
ADHYIATEDEGWETKYFDTLDFMVLCGNSLSAVDFGKLPKILKAQGVIASISVPDQH
EAIHLHPAQLLGFQITQSCLASRAEIIEMLNLVAEKKIKLWVETVPISEENISTVFKRM
ESGDVRFRFTLTDFDKQFDQFKAKK
SEQ ID NO: 22 (Danio rerio) (B8A539)
MMLALVAFAAGLGLVALILRLLSPQIRKYAAGGSCRSTVRLDGKVALVTGANSGIG
KETALDLASRGARVILACRDLEKAEEAAAEIRTRVGGAKVEVRELDLADCCSIRAFA
QRFLREVDHLHILINNAGVMMCPYMKTADGFEMQIGVNHLGHYLLTYLLIGLLKRS
APSRIVVVSSLAHNFGWIRFHDLHSQGSYNSGLAYCQSKLANVLFTRELARRLQGSN
VTVNSVHPGTVRSELVRHSTLMSLLFAFFSMFLKSPKEGAQTSIYCAVAEELQSISGK
HFSDCAPAFVAPQGRSEETARKLWDVSCELLGIEWD
SEQ ID NO: 23 (Gasterosteus aculeatus) (G3PFC0)
MLLLLIVAGLGVVTLLVILFAPHIRKYAAGGVCSSTASLDGKVALITGANTGIGKETA
LELAVRGARVIMACRNVDKGEEAADSIRAAHPEAQVEVRELDLADTCSIRAFAQKFL
GEVNSLHILINNAGVMMCPYTKTIDGFEMHIGVNHLGHFLLTSLLIGLLKRSAPARIV
VVSSLAHNFGWIRFHDLHSQGSYNSGLAYCQSKLANVLFTRECARKLKGTDVTVNS
VHPGTVNSDLTRHSTLMTIFFTVFSTFLKTPREGAQTSIYCAVADELHSISGKHFSDCA
PAFVAPQGRSEDTARRLWAASCELLGIEWD
SEQ ID NO: 24 (Latimeria chalumnae) (H3BEA6)
MCFWMFTGISALVGFLLSVLRPYIRRFAAGGVCKSNARLDGKTVLITGANTGVGKET
MRDLARRGARVIAACRDVSKGEAAVNEIRMDTGNEQIVVRKLDLADTRSIREFADN
LLREEKALHILINNAGVMMCPYSKTEDGFEMQLGVNHLGHFYLTYLLLNLLKHSAP
ARIINLSSLAHNLGWIKFADLQSEKSYNSGIAYCQSKLANVLFTRELARRLQGTKVTA
NSVHPGTVHSELTRHSFMVSLAWKIFSVFIKTPMEGAQTSIYCAVAEELESISGKYFS
DCSPAFVAPQGRNDETAKRLWDISCKLLAIKWD
SEQ ID NO: 25 (Tetraodon nigroviridis) (H3DMU7)
MLLLLIVAGLGVVTLLVVLFKPQIRKYAAGGVCQSTATLYAKTVLITGANTGIGKET
ALDLATRGARVIMACRDVDKGEEAAASIRAACPKATVEVRELDLADTCSIRAFAQKF
LREVNQLHILINNAGVMMCPYTKTIDGFEMHIGVNHLGHFLLTHLLIGLLKRSAPARI
VVVSSLAHNFGWIRFHDLHSQGSYNSGLAYCQSKLANVLFARELARRLKGTEVTVN
SVHPGTVNSDLTRHSTLMTIFFTIFAMFLKTPQEGAQTSIYCALAEELHSISGKHFSDC
APAFVAPQGRSEETARRLWQASCELLGIEWD
SEQ ID NO: 26 (Oreochromis niloticus) (13KUB2)
MMFLLLIIAGLGVVTLLVILFAPQIRKYAAGGVCKSTTRLDGKTVLITGANTGIGKET
ALDLAMRGARVIMACRDVEKGEEAAASIRASYPEARVEVRELDLADTCSIRAFAQK
FLREVNQLHILINNAGVMMCPYTKTVDGFEMHIGVNHLGHFLLTSLLIGLLKRSAPA
RIVVVSSLAHNFGWIRFHDLHSQGSYNSGLAYCQSKLANVLFTRELASRLKGTNVTV
NSVHPGTVNSDLTRHSTLMTILFTIFSVFLKTPREGAQTSIYCAIAEELHSISGKHFSDC
APAFVAPQGRSAETARRLWDVSCELLGIEWD
SEQ ID NO: 27 (Xiphophorus maculatus) (M4AK03)
MFVLLIIAGLGVVTLLVILFAPHIRRYAAGAVCKSTARLDGKTVLITGANTGIGKETA
LDLAVRGARVIMACRNVEKGEEAAASIRAAYSEAKVEVRELDLADTCSIRAFAQKFL
REVNHLHILINNAGVMMCPYTKTVDGFEMHIGVNHLGHFLLTYLLIGLLRRSAPARV
LVVSSLAHNFGWIRFHDLHSQGSYNSGLAYCQSKLANVLFARELARRLKGTNVTVN
SVHPGTVNSDLTRHSTLMMILFTVFSVFLKTPLEGAQTSVYCAVAEELHSVSGKHFS
DCAPAFVAPQGRSEETASKLWDVSCELLGIDWD
SEQ ID NO: 28 (Anolis carolinensis) (R4GAG6)
MQEWLGGALALGLALLWLLAAPSIRKYISGGKCTSTAKLNGKVVVITGANTGIGKE
TARDLAGRGARVILACRDMEKAEAAANEIRTKTGNQQVIAKKLDLADTKSIREFAEN
FLKEEKELHILINNAGVLLCPYSKTVDGFEMQFAVNHFGPFLLTFLLIERMKESAPSRI
VNVSSLAHCLARIRFEDLQGEKSYHRGLAYCNSKLASILFTRELARRLQGTRVTANA
LHPGSIVSELGRHLTILIFLGKLLTFFLKTPQEGAQTSVYCAVAEELESVSGKYFSDCK
PAYVWPQGCDDETAKKLWDVSCELLGIQWD
SEQ ID NO: 29 (Anolis carolinensis) (R4GC18)
MQDFPGCALALGLALALPLLGLLAAPYIRKYFAGGKCTSTAKLNGKVVVITGANTGI
GKETAKDLAGRGARVILACRDMVKAEAAASEIRTKTGNQQVIAKKLDLADTKSIREF
AENFLEEEKELHILINNAGVMMCPYSKTADGFEMHFGVNHLGHFLLTFLLTECLKKS
APSRIVNVSSLAHHGGRIRFEDLQGEKSYQWGLAYCHSKLAGILFTRELARRLQGTG
VTVNALHPGTVASDLPRHSTIMNFLWKLLPFLLKTPQEGAQTSVYCAVAEELGSVSG
KYFSDCKPAYVSPQGRDDETAKKLWDVSCELLGIQWN
SEQ ID NO: 30 (Fundulus heteroclitus) (A0A146ZLA9)
MVSPEQKVVLITGCSSGIGLRMAVMLAKDEKKRYHVIATMRDLKRKDKLVEAAGD
AYGKTLSLAVLDVCHDESVKQCIDSIKDRHVDVLINNAGIGLVGPIESIPIEEMKKVFE
TNFFGVIRMIKEVMPDMKKRRAGHIIVVSSVMGLQGVVFNDVYAASKFAMEGFCES
LAVQLLKFNVILSMIEPGPVHTEFEAKMIQDVKQKEYPGADPDTVHYFKNVYLPSSV
DIFEALGQTPDDIARCTKKVIEASRPRFRNLTNPLYTPIVALKYADETGGLSVHAFYH
MLFNMGPLMHVSMTAMKYLTCGCLRRRTISPN
SEQ ID NO: 31 (Xenopus laevis) (A0A1L8GVT0)
MAGDSKKTVLVTGCSSGIGLKIAVQLAKDPQERYYVIATMRDLGKSGKLEAEVGEA
LNKTLVIRQLDVTKEDSVKKCLDDLPGQRVDILVNNAGVGQIGPIESFSMEDMRKVF
ETNFFGVVHLIKKVLPGMKKRGGGHIVVISSIMGLQGVIFNDIYAASKFAIEGFCECL
AVQLLKFNIFVSMIEPGPVNTEFEMKLMEEVSRSDFPGSDADTIRYFKDIYLPSAHEIF
SSMGQTPEAVAKATVKVIGMQQPVFRHQTNVLYTPLTALKYADNTGNLSVRTFYNL
LFNYGTLFHLSLGFLRCITCNCFRRRVMPV
SEQ ID NO: 32 (Xenopus laevis) (A0A1L8H2R2)
MAGDSKKTVLITGCSSGIGLQIAAQLAKDPQERYYVIATMRDLGKSGKLEAEVGEFL
NKTLVIRQLDVTKEDSVKHCLDALPGQRIDILVNNAGVGQIGPIESLSMEDMRKVFET
NFFGVVHLIKAVLPGMKKRGSGHIVVISSVVGLQGVIFNDIYSASKFAIEGFCECLAV
QLLKFNIFVSMIEPGPVNTEFEMKLMEEVSRSDFPGADADTIRYFKDIYLPSAHEIFSSI
GQTPEAVAEATVKVIGMRQPVFRHQTNVLYTPLTAMKYADNTGNLSIRTFYNLLEN
YGTLFHFSLGFLRCITCNCFRRRVMPV
SEQ ID NO: 33 (Scleropages formosus) (A0A1W5A3B1)
MASDGQKVVLITGCSSGIGLRVAVLLAKDDRKRYHVIATMRDTKRKDKLLEAAGDT
YGKTLTLQTLDVCSEESVKQCINSIKDRRVDILINNAGIGLLGPVESISIEEMKKVFETN
FFGMVRMIKEVMPDMKMRRSGHIIVMSSVMGLQGVVFNDVYTASKFAMEGFCESL
AVQLLKFNVNVSMIEPGPVHTEFEVKMMQDVAKMEYPGADADTVRYFKDVYLPSS
LDIFEAMGQSPEDIARCIKKVIEARRPRFRNLTNSLYTPIVAMKYADETGGLSVHTFY
NLLFNFGPLMHITMNILKCLTCSCLRRRTISPN
SEQ ID NO: 34 (Cyprinus carpio) (B5UAY7)
MASGGEQKVVLITGCSSGIGLRIAVLLARDELKRYHVIATMRDLRKKDRLVEAAGDT
YGQTLTLLSLDVCSDDSVRKCIDSVKDRHIDILINNAGVGLLGPVESISMDEMKKVFE
TNFFGTVRMIKEVMPDMKKRRAGHIIVMSSVMGLQGVVFNDVYTASKFAIEGFCES
MAVQLLKFNVKLSLIEPGPVHTEFETKMMEEVAKMEYPGADADTVRYFKDVYIPSSI
DIFEALGQTPEDIAKCTKKVIESSRPRFRNLTNSLYTPIVAMKYADESGGLSVHTFYN
LLFNFGSLMHITMSILKCLTCSCLRRRTISPD
SEQ ID NO: 35 (Danio rerio) (E7F0C2)
MGTRKVLVTGCSSGIGLAVAVRLAKDELRRFKVVATMRDLDRREALERAAGETLN
RSLEIRQLDATCEDSIRDCVNSLPDRQVDVLVNNAGVGMIGPLECQSMSSMQDLENT
NFFGLVRLVKELLPQMKKRQSGHIIVMSSVLGIQGLLENDLYAASKFAVEGFCESLA
VQAMKFNVKMTLVEPGPVVTEFERKVYEEAETMDLSETDEETAQIFRQIYLPYSRRV
FHSIGQTPEEVAEQTTRLILSKNPPFRHQTNRLYMPLTAMKHADPTGRLPIDAFYKMI
FQHDRIFSASLNIMRILNRKIGKSAA
SEQ ID NO: 36 (Gasterosteus aculeatus) (G3P670)
MANSGQKVVLITGCSSGIGLRIAVTLAKDEKKRYHVIATMRDLKKKDKLLEAAGDV
YGKTLMLLPLDVCSDESVKQCINNVKDRHIDILINNAGVGLLGPLESISIEEMKRVFET
NFFGVVRMIKEVMPDMKKRRSGHIVVMSSVMGLQGVVFNDVYTASKFAMEGFCES
MAVQLMKFNIRLSMIEPGPVHTEFETKMMEDVAKMEYPGVDADTVRYFKDVYLPS
STDIFEAMGQTPEDIAKCTKKVIESNSPRFRNLTNSLYTPIVALKYADETGGLSVNTFY
NLLFNFGPLMHITMSILKCLTCSCLRRRTVSPN
SEQ ID NO: 37 (Gasterosteus aculeatus) (G3PW42)
MASPGQKVVLITGCSSGIGLRMAVMLAKDQQQRYHVIATMRDLKRKDKLVEAAGD
AYGKTLSLAVLDVCTDESVKRCIDGIKDRHVDVLVNNAGIGLVGPIESIPIDEMKKVF
ETNFFGVIRMIKEVMPDMKRRRGGRIVVVSSVMGLQGVVFNDVYAASKFAMEGFC
ESLAVQLLKFNVTLSMIEPGPVHTEFEAKMIQDVKQKEFPGADADTVNYFKNVYLPS
SVDIFETLGQTPDDIARVTKKVIEASSPRFRNLTNPLYTPIVALKYADETGGLSVHAFY
HMLFNLGPLMHVSMTAMKYLTCGCLRSRTVAPN
SEQ ID NO: 38 (Oryzias latipes) (H2LMU2)
MANSGQKVVLITGCSSGIGLRIAVTLASDEKKRYHVIATMRDLKKKDKLLEAAGDV
YGKTLMLLPLDVCSDESVRQCINSVKDRHIDILINNAGVGLLGPVESISIEEMKRVFDT
NFFGVVRMIKEVMPDMKKRRSGHIVVMSSVMGLQGVVFNDVYTASKFAMEGFCES
MAVQLLKFNIRLSMIEPGPVHTEFETKMMEEVAKMEYPGVDADTIRYFKDVYLPSSI
DIFEAMGQTPEDIAKCTKKVIESDSPRFRNLTNSLYTPIVALKYADETGGLSVNTFYN
LLFNFGPLMHITMNILKCLTNRTIKRYQSTKTEPS
SEQ ID NO: 39 (Oryzias latipes) (H2M0R5)
MVSPGQKVVLITGCSSGIGLRIAVMLAKDEKKRYHVIATMRDLKRKDKLVEAAGDT
YGKTLSLAVLDVCSDESVKQCINGIENRHVDILINNAGIGLVGPLESISIEEMKKVFET
NFFGVVRMIKEVMPDMKKRKDGRIIVVSSVMGLQGVVFNDVYSASKFAMEGFCESL
AVQLLKFNVIVSMIEPGPVHTEFEVKMIQEVKQRDFPGADAETVRLFKEVYLPSSVDI
FEALGQTPDDIARCTKKVIEASSPRFRNLTNALYTPIVAMKYADETGTMSVHAFYHL
LFNLGPLMHVSVMAMKYLTCGCLRRRTISPN
SEQ ID NO: 40 (Fugu rubripes) (H2UL76)
MASPGQKVVLITGCSSGIGLRMAVMLAKDPQKRFHVIATMRDLKRKDKLLEAAGDT
YEKTLSLAVLDVCSDESVKQCINGIKDRHVDVLINNAGIGLVGPVESISIEEMKKVFE
TNFFGVIRMIKEVMPDMKKRRGGHIIVVSSVMGLQGVVFNDVYAASKFAMEGFCES
LAVQLFKFNVNLSLIEPGPVHTEFEAKMMQDVRQKDFPGTDPETMHYFKNVYLPSAI
DIFEMFGQTPDDIARCTVKVVESSRPRFRNLTNPLYSPIVALKYADDTGNLSVRAFYH
MLFNMGSVMHVSMTVMKYLTCGCLRSRTISPN
SEQ ID NO: 41 (Takifugu rubripes) (H2UZY9)
MTQKVVLITGCSSGIGLSLAARIAKDEKKRFMVYATMRNLSKGEALVKAAGRALGR
TLEIKQLDVCDEDSIRACVDSLPDRRVDILISNAGMGLIGPIECQSIGEMKTVMDTNFF
GLVRLVKEILPDMKRRKRGHIVVISSVMGIQGILFNDIYAASKFAVEGFCESLAVQAL
RFQLNISLIEPGPVITEFERKVYEEGLKTDLSKADEVTADMFTNIYLKNYKQIFETLGQ
TADDIAEHTLKIITMDNPPFRHQTNTLYTPMTTLKYADPNGDLPIDTFYKMVFEHDKI
FNASLNFLKLLRWRSRKSFTLEKDNNN
SEQ ID NO: 42 (Anolis carolinensis) (H9GCI2)
MAVRNVLVTGCSSGIGLALAVRLARDELKRFRVIATMRNLAKRQALEMQAGPVLEK
TLEIKELDVTSEESIRHCVDSIPQRRVDVLVNNAGLGMIGPVESQSLTSMQHLFDANF
FGLVRLVKEVFPDMKKRKQGHVVVMSSVLGLQGLLFNDIYSASKFAVEGFCESLAL
QALKFGVNISLIEPGPVITEFERKLYEEAANMDLSGLDKETLDIFQGFYMAYSKEVFT
ALGQSPEEVAEHTMKVITSDQPPFRYQTNSLYTPITTLKHADPSGNLPLNAFHQMIFQ
HDRLFKASLSLLKMLQWRKRRD
SEQ ID NO: 43 (Anolis carolinensis) (H9GI25)
MAKATPRTVLITGCSSGIGLRMAVQLAQDPGKRFHVIATMRDLRKKDKLEAAAGDT
LNKTLTIQRLDVCNDESVTECINSLPGKQLDVLVNNAGVGLVGPVESISIDDMKRVFE
TNFFGAIRMIKAVLPEMKKRQKGHIVVISSVMGLQGVPFNDVYAASKFAMEGFCESL
AVQLLKFNIFVSMVEPGPVNTEFEMKLMEEVARSEFPGADAATVRYFKEVYLPASH
EIFTTMGQTPESVAKAVVNVIAKERPPFRTQTNTLYTPLVALKYADTSGDLSVGTYY
NLLFRFTGLFHLSMSCLKCITCSCFRRRVTPA
SEQ ID NO: 44 (Oreochromis niloticus) (13IZM1)
MANSGQKVVLITGCSSGIGLRIAVILAKDEKSRYHVIATMRDLKKKDKLVEAAGDA
YGKTLTLLPLDVCSDESVRQCVNNVKDRHIDILINNAGVGLLGPVESISIEEMKKVFE
TNFFGVVRMIKEVMPDMKKRRSGHIIVMSSVMGLQGVVFNDVYTASKFAMEGFCES
LAVQLMKFNIRLSMIEPGPVHTEFETKMMEDVAKMEYPGADADTVRYFKDVYLPSS
IDIFEAMGQTPDDIAKCTKKVIESNNPRFRNLTNNLYTPIVALKYADETGGLSVNTFY
NLLFNFGPLMHITMSILKCLTCSCLRRRTISPN
SEQ ID NO: 45 (Oreochromis niloticus) (13IZP7)
MATRRVLVTGCSSGIGLAVAARLAKDELRRFKVVATMRDLGKRGPLEKAAGDSLN
KTLEIKELDACCETSIKECINSLPNRRVDVLVNNAGVGMIGPLECQSIDAMKELFDTN
FFGLVRLVKEVLPDMKQRQSGHIVVMSSVMGIQGLLENDVYSASKFAVEGFCESLA
VQAMKFNIKTTLVEPGPVVTEFERKMYEGAEKMDLSGTDEETARIFRELYLPYSKEV
FTSLSQTPEEVAQQTVNVITAKDPPLRHQTNKVYMPMTALKHADPTGRLPLDTFYK
MIFKHDRVFSATLGVLRLLQRRAEQIKAIYSFFLSKSVYHFIS
SEQ ID NO: 46 (Xiphophorus maculatus) (M3ZJL7)
MGTRRVLVTGCSSGIGLAVAVRLAKDETKRFKVVATMRDLEKRGPLEKAAGDLLN
KTLVIQKLDACCEESIKECINSIPNRQVDVLVNNAGIGMIGPLECQSITSMKELFEVNF
FGLVRLVKEVLPDMKQRQSGHIVVMSSVMGIQGLLFNDVYSASKFAVEGFCESLAV
QAMKFNIKTTLVEPGPVMTEFERKVYEDAEIIDLSETDEETARIFRDIYLPYSRKVFTT
LGQTPEDVAEQTQKVITAKEPPLRHQTNRLYMPMTALKHADPTGRLPLEAFYKMIFK
HDTVFNATLGILRMMNRRKGNK
SEQ ID NO: 47 (Xiphophorus maculatus) (M3ZLM4)
MNQKVVLITGCSSGIGLALAARIAKDEKKRFMVYATMRNLGNAEPLVEAADRTLGR
TLELKQLDVCSEESIKACVDSLPERRVDILISNAGMGLIGPIECQSIDEMKTVMDTNFF
GLVRLLKEILPDMKKRKKGHIVVISSVMGIQGILFNDVYAASKFAVEGFCESLAVQA
LRFNLNISLIEPGPVITEFERKVYEEGLKTDLSKADKVTADMFTNIYLKNYKQIFESLG
QTAEDVAEHTHRIITMDDPPFRHQTNTLYTPMTTLKYADPNGDLPIETFYKMVFEHD
KVFNASLNFLKLLRWRSRKSFTLEKDKST
SEQ ID NO: 48 (Xiphophorus maculatus) (M4AEE7)
MVSPEQKVVLITGCSSGIGLRMAVMLAKDEKKRYHVIATMRDLKRKDKLVEAAGD
AYGKTLSLAVLDVCSDESVKQCINGIKDRHVDVLINNAGIGLVGPVESISIEEMKKVF
ETNFFGVIRMIKEVMPDMKQRRGGHIIVVSSVMGLQGIVENDVYAASKFAMEGFCES
LAVQLLKFNVILSLIEPGPVHTEFEAKMIQDVKQKEYPGTDPDTLHYFKNIYLPSSVDI
FEAFGQTPDDIAKCTKKVIEASRPRFRNLTNPLYTPIVALKYADETGGLSVHAFYHML
FNMGPVMHVSVTAMKYLTCGCLRRRTISPN
SEQ ID NO: 49 (Xiphophorus maculatus) (M4ATJ1)
MANSGQKVVLITGCSSGIGLRIAVTLAKDEKKRYHVIATMRDLKKKDKLLEAAGDT
YGKTLMLLPLDVCSDESVKECINNIKDRHIDILINNAGVGLLGPVESISIDEMKRVFDT
NFFGVVRMIKEVMPDMKKRRSGHIVVMSSVMGLQGVVFNDVYTASKFAMEGFCES
MAVQLMKFNIRLSMIEPGPVHTEFETKMMEDVAKMDFPGVDADTVRYFKDVYLPS
SIDIFEAMGQTPEDIAKCTKMVIESERPRFRNLTNSLYTPIVALKYADETGGLSVNTFY
NLLFNFGPLMHITMSILKCLTCSCLRRRTISPN
SEQ ID NO: 50 (Danio rerio) (Q6PC70)
MASGGGQKVVLITGCSSGIGLRIAVLLARDEQKRYHVIATMRDLKKKDRLVEAAGE
VYGQTLTLLPLDICSDESVRQCVNSVKDRHIDVLINNAGVGLLGPVESISMDEMKRV
FETNFFGTVRMIKEVMPDMKKRQAGHIIIMSSVMGLQGVVFNDVYTASKFAIEGFCE
SMAVQLLKFNVKLSLIEPGPVHTEFETKMMEEVAKMEYPGADPDTVRYFKDVYVPS
SIDIFEAMGQTPDDIAKCTKKVIETSQPRFRNLTNSLYTPIVAMKYADETGGLSVQTF
YNLLFNFGSLMHISMSILKCLTCNCLRRRTISPD
SEQ ID NO: 51 (Danio rerio) (Q7T218)
MASGGGQKVVLITGCSSGIGLRIAVLLARDEQKRYHVIATMRDLKKKDRLVEAAGE
VYGQTLTLLPLDICSDESVRQCVNSVKDRHIDVLINNAGVGLLGPVESISMDEMKRV
FETNFFGTVRMIKEVMPDMKKRQAGHIIVMSSVMGLQGVVFNDVYTASKFAIEGFC
ESMAVQLLKFNVKLSLIEPGPVHTEFETKMMEEVAKMEYPGADPDTVRYFKDVYVP
SSIDIFEAMGQTPDDIAKCTKKVIETSQPRFRNLTNSLYTPIVAMKYADETGGLSVQTF
YNLLFNFGSLMHISMSILKCLTCNCLRRRTISPD
SEQ ID NO: 52 (Gadus morhua) (UPI00023F1C4D)
MANSGQKVVLITGCSSGIGLRIAVTLAKDEKKRYHVIATMRDLKKKEKLVEAAGDT
FGKSLTLHPLDVCSDDSVSQCINSVKDRHIDILINNAGVGILGPVESISIDQMKKVFET
NFFGVVRMIKEVMPDMKARRSGHIIVMSSVMGLQGVVFNDVYTASKFAMEGFCEC
MAVQLMKFSVRLSMIEPGPVHTEFETKMMADVAKMEFPGVDADTVRYFKDVYLPS
SIDIFEAMGQTPEDIAKCTKKVIESSSPRFRNLTNSLYTPIVALKYADETGGLSVNTFY
NLLFNLGPLMHITMSILKCLTCSCLRRRTVSPN*
SEQ ID NO: 53 (Gadus morhua) (UPI00023F3DEC)
MASNGQKVVLITGCSSGIGLRMAVMLANDEQKRYHVIATMRDLKRKDKLVEAAGE
AYGKTLSLAVLDVCTDESVKQCIDGIKDRHVDVLINNAGIGLVGPLESIPMEEMKKV
FETNFFGVIRMIKEVMPDMKKRRGGHIIVISSVMGLQGVVFNDVYAASKFATEGFCE
CLAVQLLKFNVTLSMIEPGPVHTEFEAKMIQDVRQKEYPGTDPETVNYFKNVYLPSS
VDIFETLGQTPDDIARVTKKVIEASSPRFRNLTNPLYTPIVALKYADETGGLSVHAFY
HMLFNLGPLMHVSMKAMQYLTCGCLRSRTISPN*
SEQ ID NO: 54 (Xenopus laevis) (UPI00084DD5CA)
MAGDSKKTVLVTGCSSGIGLKIAVQLAKDPQERYYVIATMRDLGKSGKLEAEVGEA
LNKTLVIRQLDVTKEDSVKKCLDDLPGQRVDILVNNAGVGQIGPIESFSMEDMRKVF
ETNFFGVVHLIKKVLPGMKKRGGGHIVVISSIMGLQGVIFNDIYAASKFAIEGFCECL
AVQLLKFNIFVSMIEPGPVNTEFEMKLMEEVSRSDFPGSDADTIRYFKDIYLPSAHEIF
SSMGQTPEAVAKATVKVIGMQQPVFRHQTNVLYTPLTALKYADNTGNLSVRTFYNL
LFNYGTLFHLSLGFLRCITCNCFRRRVMPV*
SEQ ID NO: 55 (Gibberella fujikuroi) (Q5GN50)
MKFLQQNSFTQTSMSQPHEDVSPAIRHPYLTGNFAPIHKTTNLTPCTYSGCIPPELTGG
QYVRNGGNPVSHQDLGKDAHWFDGDGMLSGVAFRKASIDGKTIPEFVNQYILTDLY
LSRKTTSIASPIMPSITTLVNPLSTMFQIMFATFRTIFLVILSNLPGSQQAIKRISVANTA
VLYHDGRALATCESGPPMRIQLPSLDTVGWFDGVEAEGEPEISQAGSDDSPFGGSGIF
SFMKEWTTGHPKVDPVTGEMLLYHNTFMPPYVHCSVLPKSNEKAPGHRLVNQPVL
GVSGARMMHDFGASRSHTIIMDLPLSLDPLNTMKGKEVVAYDPTKPSRFGVFPRHLP
SSVRWFHTAPCCIFHTANTWDSQSSEGELSVNLLACRMTSSTLVYTAGNIRPPVRSRC
TQARVWSDEREETACRYKEAPALESPGESTGLADYFPITAESDDYDQCRLYYYEFDL
AMESRNHVKSQWALSAIPFEFPSVRPDREMQEARYIYGCSTSTSCFGVALGRADKVD
LLVKMDAKTLIQRGKKMNATSITGCVDRRSVCEILQEQRKDDPIYIFRLPPNHYAQEP
RFVPRACSTEEDDGYLLFYVFDESQLLPSGDCPPSATSELWILDAKNMRDVVAKVRL
PQRVPYGLHGTWFSSQDIESQRSVESLRSLEVVQRKKEEWVNSGGQIRKSWMVLRE
KLEKAVG
SEQ ID NO: 56 (Ustilago maydis) (strain 521/FGSC 9021) (A0A0D1E9J4)
MVKGSSNRRQHSASLQGLPSSQHCAPVISIPSPPPPAEDHAYPPSSFTIPLSKDEELAEA
GPSRPGSSAISRRPVLSRRRTSKKEYVHPYLSGNFAPVTTECPLTDCLFEGTIPEEFAGS
QYVRNGGNPLANSERDRDAHWFDADGMLAGVLFRRTPKGTIQPCFLNRFILTDLLLS
TPEHSRLPYVPSIATLVNPHTSVFWLLCEIIRTFVLAMLTWLPGLGLGGNQKLKRISV
ANTSVFWHDGKAMAGCESGPPMRIMLPGLETAGWYTGEEDKEKETCDKNSGNSLT
SSSSKGFGGGPPIVSMLREFTTAHPKIDPRTQELLLYHMCFEPPYLRISVIPASQSKKTD
LPAHAKTIKGKAVRGLKQPKMMHDFGATATQTVIIDVPLSLDMMNLVRGKPILHYD
PSQPTRFGILPRYEPERVRWYESAEACCIYHTANSWDDDGKFDASHEHATRSAIRGV
NMLGCRLNSATLVYSAGNLLPPSHVLPPPNCPEKCQLYYWRFDLEHAETNTISHEFA
LSDIPFEFPTINEDYSMQQACYVYGTSMRDGTFDAGLGKAAKIDALVKLDAQALIRK
GKAMWSQGRLKAGDSVDTRTVEEVLTAQRDGSASPEDPIKIFEMPRGWYAQETTFV
PRRSSTNETSQEDDGWLVCYVFDEATGLHPSTGEVLPGASSELWIIDAKLMSRVVCR
IKLPQRVPYGLHGTLFTEEQIASQKPIDPSQVRSWALSINLADPFSSSALGSTVYSAAG
KAATSKFKNREETYAAFIKDPIRIGAWWVKRNIELLIA
SEQ ID NO: 57 (Dokdonia sp. MED134) (WP_021779399.1)
MKNIRIVITFFLLWLSIQFNSTIEDIIAYVAIFTLGLVHGANDIKLLEKNVSSSPNHKYVI
LGRYLLIVVAIAILFYVIPSLALILFILVSGFHFGEEHWDKLIVTKGNGHYLFYTAYGL
LILFLIFYFNSEETTEIISAITGFTPAKFYYLVGLIFFAILSILSFIVAKAQMNTTSTILQEI
VHLIILAIVFKMATLLLSFALYFVVWHAIPSMKSQIIYLYGNTSQKSIVGYLRSSLAYW
LLAITSISIFYYYLGDKTYLFNAIFFAFLAAITFPHVWIINSLRK
SEQ ID NO: 58 (Actinobacterium SCGC AAA278-022) (UPI0003699A87)
MEMAKLKTFSRVRTFSSAIVAVAIALSIVFSSWLGVDSLNWQVVMAVVALAIGIPHG
ALDHLVTLPKAQPLKMAIFIAIYVAIALIAIWAILQWNVWGFIAVVIMSATHFGIGDS
AFISELNRLKGIQSHLPIWAYAPAAGALPVVIPLVNSRSTDALQKVNSELINWHHGYT
SELQIAVAVIATLSAMTLLSKKRYRDLLDVALLAALASVAPPLVAFAVYFGCWHAM
RHTARLSSLLPRSLAAYEAGNSWQAFRSAVIPGLPALIGTLIFVALLAGFSHNNVSDS
FLWLTLVTIWALTVPHMMVTAKLDRAALKNKSHLR
SEQ ID NO: 59 (Salinibacter ruber) (WP_011404936.1)
MHNPVTLRSPRIYGSVLALTLLAVAVGAWGSVSLGPWALGVLFVAVVLTGMPHGAI
DHLVAARLWGLDPTWADQAKFYGGYLVLMALYGALWIVAPAWSLGLFLVMTMY
HFGQADLAYWRLPPLSARLLYLSRGLFLLGLPIVAFPEVVAPIFEAMGGVQLLEWPG
VTTAPGALFAGLVAQHLGALGIGRLGADPSVDWALGREATNVAVLAALFGLVHPLV
AFAVFFGGWHALGHILELLRFFRRQGDAMSMAQFYREAFLFTAIPFVGLAGLYAAT
QSFGLEDQMVALLFILIAIMTLPHMVIVEKMYREREKGAPQAVS
SEQ ID NO: 60 (Zymoseptoria tritici ST99CH_3D7) (A0A1X7SAA9)
MVHAGQKRKRSPSENIPPTPQPRHPYLTGNFAPIQQTLALTPCTYTGQIPEELADGEY
VRNGGNPVSNDDLGRDAHWFDGDGMLSGVAFTKDEATGRIQPEFVNQFILTDVYLN
TLASPKLRVPILPSIATLVNPLASFIWVTIRIFRTILLVILSHLPGSKQAIKKISVANTNVV
YHDGRALATCESGPPMRIQLPGLETVGWYDGANAQGEPKKEDIAKEATLGQDSGLIS
FMREWTTAHPKVDPTTKEMLMFHSSFAPPYVQYSIVPKSSESEGGEGQTRRSKILNA
AVPGVTSAKMMHDFGVSPTHTVIMDLPLSLDPINQLKGLPTVTYTPSKPSRFGVFPRR
QPESVQWFETDASCIFHTANTWDDVSSAGETTAVNMLACRLTSATLVFAAGNIAPPI
EKLKAPPVKAKRRMPFFSKYDSATEVTIHDLDSPTDEKEPLLTVTTVDEAGEVEEPFE
EDQCRLYYYHFDMTTHNITHQWALSSIPFEFPSVRPDVEMQNAQYIYGCSTTTTSFGS
ALGKATKIDALVKIDAKTLIERGRRTPPPSVTGVVDSRNMTQVLADQHDPTDPVRVF
TMPEGWFAQEPRFVPSSSNASEDDGYLLFYAFDEGQLSSLGDVPDDVSPSRAKSELW
IVNAKNMKDVVARVQLPQRVPYGLHGSWFTAEQIAEQKSVECIRTTAKALEAREKG
VWMSVRSWVEGVLG
SEQ ID NO: 61 (Planctomycetes bacterium) (MBA4684017.1)
MNLLWIPLLAILLVGIPHGALDAILIRKVSGSRSSIWFYSSYLLLCVAVVAIWFQAPVV
SLAFFLLISLWHFGGADARGYGDDRWLSRLVHGSMIPIVLPALQPEAVAPLFRLVAL
QNETPFVQWAQILIWPWAALLLLKVIRKEVCKREVLELGGLSLFFWLADPIYSFALYF
CGLHSMRHFQQSFQFLSRNRFGKKETIEFAILSGVPILMILLGGAMLDETQWLQGLTG
SVFIGLAALTVPHMILIDGWVPLGKPDSGHST
SEQ ID NO: 62 (Halieaceae bacterium) (HBZ50032.1)
MLPDLYTLYLPIALMTFIGVPHGALDGYLLHSHSDSKRGTFALLLAYLGIALACVLF
WWWQPTLALLSFLLLSLYHFGRSDHQQTGDSVSLTAWIAHGGLWALFLPWQQADA
TAVLFQALKTDPVILEAFLAPATAIWLLVSGLYLCRQWRNKDQQWRVWVISATAIV
LLPPLWSLCLYFCLWHARRHTQWVLRYAKSPSEAKLWMLSIFSVTLAMGAIAYSVL
LPSTAIETLTARIFFIGIFALTVPHMILIDYYLGHLHDQQPSL
SEQ ID NO: 63 (Porticoccaceae bacterium) (MAE55128.1)
MTTSDIFALALIVFLGIPHGSFDGVIARRLGWSNNLIEQMSFNVIYLSIAILVVIVWFY
MPLISLSIFLVISGIHFGMSDISKIQNTGSSYYFPLISHAGLIPVALPVIQKDYVIEVFTIL
SNNTASKIIFSTLETIYPLYLVIVSAYFFYSIYNKKWLFNAVSLIMLILLAFLIPPLLTFA
LYFCFWHSRGHILRVWQRVKINERRSHIIETVGYTIFTWLILASGLWLLDGSLNESIIK
MVFIGLAALTVPHMILVDFWANRRKVL
SEQ ID NO: 64 (Gammaproteobacteria bacterium) (RCL40986.1)
MLELGLLNTIALISIIVLGIPHGGLDAAISRKKGWSKTKINTIYFHVFYLSLTSLIVALW
YFFPLISLILFLTISALHFSYSDLNNSKDIRIDALISHGGIVPIIIPYFQADLVLEFFSILSGQ
ENAAIVITIINYLIFPWAAFFVIYTFNVIRKKKYSFNYTQLLLMLILALILPPLVSFSIYFC
IIHSPRHIFIILNKMNSHERLRAIKETTFYSLTAFLLMFIVGYFLASSYIITDSIIKVVFIGL
AALTVPHMILIDYFESLEKE
SEQ ID NO: 65 (Euryarchaeota archaeon) (MBJ64590.1)
MSLTTALSSMDTLNLIALMCVVFIGLPHGAMDGALAAHFGWMESKKKAATFLIGYV
AMAALVVGFWFVAPAISFIIFLGISMYHFGKGDVNTDGEEPSVTESLARGGVVIGGIS
QFHRVDADEIFQVLIGSNTELVWLFLNSVVPMTLIFAILSMVNKTTGERGSLLAEIAG
LALIFSILPPLVGFAIYFCLIHSIRHFASMRAMLSDTITKLQITKTTILFSAISWAAGLIIL
AQQSSNIGFEPALLQVIFIGLAALTVPHMILVDGYTEYQKSQ
SEQ ID NO: 66 (Alphaproteobacteria bacterium) (GIR97087.1)
MMDILSVTDLFALAAVMLIGLPHGAFDGALAFCLGFGRSPGKIIGFLVMYLLLAGLS
ALIWLVSPAFALAAFLVLTIVHFGSGDTEHLYQLGPRLVQRSLKACQILVHGGMVTIL
LPVFHTEEVSQLFIVLAGPNAVLIMDALRPALTIWLAAACIYAGAALFNRQYGAAAG
ELAGLAVLVWLLPPLAGFAVYFCVVHSRRHFTSIWKAMQLFVSRRFILISGGILTAAS
WAMGAGLYFSQTMSGSFSPDEAFIRAVFILLAALTVPHMLLVDTMYRPTLKKITQP
SEQ ID NO: 67 (Uncultured marine bacterium HF10_19P19) (ABL60983.1)
MIDILSVTDLFALAAVMLIGLPHGAFDGALAFCLGFGRSPGKIIGFLVMYLLLAGLSA
LIWLVSPVFALAGFLVLTIVHFGSGDTEHLFQPGPRVVQRSLKACQILVHGGMVTILL
PVFHTTEVSQLFIVLAGPNAVLIIDALRPALKIWLAAACIYASAALFKRQYAAAAGEL
AGLAVLVWLLPPLAGFAVYFCIVHSRRHFTSIWKAMQLFVSRRFILISGGILTAASWA
MGAGLYFSQTMSGSFSPDEAFIRTVFILLAALTVPHMLLVDTMYRPTLKKITQP
SEQ ID NO: 68 (Henriciella sp.) (NQY41612.1)
MTWNSAPLNFLLAGMTVSIAFLLVPSLSITLQLVVLGVAVAFLGLPHGAIDAYIARRD
GLWRSTRGFAAFVGIYAVVALSVIGVWMFMPTLSLLAFLIISAWHFGADANARDQA
ERWLFGGLLLSLPSFFHPGDVASLFEAISGASAGGLVPFLQIWAPVAAIGVFAMLVRR
RPPAQQRWTDIATVAGLILFAWALPPLVYFVIYFCALHSPAHFGRVIRLVTPSDRTRA
AAYTAGFTALTLLVAGLAFIALTDKLTIQQSTLQIVFIGLAALTVPHMFLVDGICRAW
FGEAE
SEQ ID NO: 69 (Cryomorphaceae bacterium BACL22 MAG-120619-bin32)
(KRP28444.1)
MVVNNIKNYQNFKIFITVFLLWFSIQFGKPIEDSIAFVLIISIGIIHGANDLLILSLKKMK
NNRFVINLFIYLCIVSFCVIFYILNPFLAILLFIFISSYHFGEEHLSEKMITNRYFESFYYIV
YGLLIFSMLFYEALFEVNTIMLELTKTIFSEFFVKYTFIFSLISFLLMNTFLLWKEKIDA
KVFLKEGFSLGLLFLVFKTSSLILSFAIYFIFWHSIPSIINQILFISGDISKSSILFYVKKAF
LFWLISIVALFVLYQFIPEVTLLSTIVFVILFSVTAPHIWVMYKMKN
SEQ ID NO: 70 (Loktanella sp. SALINAS62) (WP_211643588.1)
MTLSPIWQTMVFAATALAMIGVDLLFQPDLMLQVLILVPAVAILGLPHGALDLPIAQ
SLWSLKGWRGTAAFSAIYLGLSLLLVAFWIAFPGPALCVFLVYSAVHFSGDWDDAG
RAQRWIGGVATVGAPALFRADEVAAIFSYLAPADAANFAAQGLAIMGAFALMSYLS
LFIFRSNFRDRAAIEIGVIWIAAACLAPLVYFIVYFCALHSVRHFTDALASLDRKKQAL
GVAIVVSGVTVLIGLLAFINLPDAGSDTLQQPIVQVVFIGLAALTVPHMILVECFQSRA
VRQPDRC
SEQ ID NO: 71 (Flavobacteriaceae bacterium) (MAH20764.1)
MKIIYSFSIVSSFMGLWITSLLPEAFELFLGFILIFTFGMVHGSNDIMIVDKLSKKTNSTF
FKTLSTYLMVVTAAILIFYFIPLLALVLFILFSAYHFGEQHWEEVLSNLILPLRKIFFFC
YGLLILYLVFLFNVEAVIEIIYEITSLKLNDIYASEVTQILFVILFLITGYAVYKKQIAIKS
VLRELFSLLIFGIIFNSSSLIWGFTIYFIFWHSIPSLLDQITFIYGDLKKKSILKYVKAALP
YWLVSLVGIAVLFLIFKHEKHFHSLFFAFIAAVTFPHVIVMLRMFSKKRI
SEQ ID NO: 72 (Robertkochia sp. 3YJGBD-33) (WP_224483742.1)
MNSYRFESYLLVLTFFSLWLLGYLNAVTARSTAILLVLTIGL VHGANDMKIISRLSEN
KQRTRKYVAGYISVFFLTSVAFYFFPLVALILFIFFSCYHFGEQHWLEHIDKPEKGTKL
FAFFYGMLIIMLILQFNAGESVMIINDLLQLELKNLPFAIFTLIALFPVLVLGYRYRKLL
FPSYEKLAKEILLMLVFIIIFNLSGLVWSFAVYFILWHSLPSLRSQLTYLYGSTTMAGV
KRYIRDSLPFWLLAIIGLGVMIWFLKDQREIIISIFFPFIAALTMPHVVIMSMMFGEEN
SEQ ID NO: 73 (Flavobacteriaceae bacterium) (NBT87107.1)
MNKNFNIIITAFVIWISLYTNAQIELLASGIFVLSLGLMHGSNDINLLQELLTKNKTKK
WVLIGFYVSLGAIVFTLAFYWRVLGLIVFMLFSSYHFGEQHLHNRLSDSKIKVFDFTL
YGLVIFSMMFYTHQNEVQFLIFQMTDWDITGFPMSYVTIGLIGLLILSWIKHYKFFKK
EIFEEMFYLVMFYIVFFNTELPLSFAVYFVIWHAIPSIKDQILVEGYTLSYKSALRYFK
QSYLYWLVSVFGIFLLYKVYDLIGNGLFPILFAALVAITFPHILLITQLFQKLNQSQKT
KEGH
SEQ ID NO: 74 (Eudoraea sp.) (MBT8293718.1)
MQNKGVDFTKLQGATLIATFFSLWLAVYFEHAVENLLAYILILSFGMLHGANDIKLL
QVEAKKKSKPYDFFRILAYYVLFVLSVAGLFYVLPSLALALFILFSAYHFGEQHWISK
INGNKLFLIFFYTAYGLVILFLLFAAHHTEVSDIIFDIIGFYIDNAYYRITLGISAFIFVAL
YLIKYKQSKINIFLELFYLLVFFIIFNTASLLWAFAIFFILWHAIPSLADQITYLYGDLSR
KHFMKYVRSSLIYWLASVITLVILFFFIDGSSEGFIPFLFSFLAAITFPHVLVISGLNKS
SEQ ID NO: 75 (Psychroflexus salaries) (SHE30376.1)
MFKIMSKLYNYIIVLNFFLVWIGFYLPEQIKTSIGLLGILSIGLIHGSNDIFLIKNIQRQQS
ILKYIFYYSLFIISFAVAFYLIPIVSLPVFILASAYHFGEQHWQKAFLKAPNYIKQLFFIC
YGNLVIGMILYFNQVESTTIIENLSGFVVNESIYLENILVSLIITSISFILCAKKINSKASTI
VLNIFYLLLFTATFYVSDLIWSFALYFVLWHSLPSLKDQIDFSFGSINFQNLLMYLKQS
FVYWLISIIGLIVFYFLLKDTKYILSILFAFIASVTIPHVFVVKKMFEKKAST
SEQ ID NO: 76 (Verrucomicrobia bacterium) (NCX47571.1)
MPVLLRQTAPWFLAALITLLSLLFPQPACTLAPWFFLSSALILGVPHGACDPWIPGWI
LHRPSRLPFLIPFFLIYLAISAAYLLLWRSYPLPSTFFFLLLTAWHWGTADASLETTPSL
RWLLFGIGRGSFVMLAPFAFHLADTWSVILLMAPNAGPPPSTFPFFPALLLALALNLF
SRPSIHQTIETLLLLLLFYLTPPLLSVGTYFVAFHAWRHLLRLSALRNQLTSNEPLFRW
THSLSQLLLLSIPLTLASLAFLRWIPPFLTSSFPKGTNKLGRPLSYSPRRPYPTPRNPCW
MDR
SEQ ID NO: 77 (Flavobacteriaceae bacterium) (UCE68985.1)
MNKKGLKIPDLRNAMLVSTFFFLWLAVYFSQSVEEVLAFVLIFSFGILHGANDLEILR
RGQAVTETGNPNVKLLLSYIGFVLGSALLFSLLPLVALTFFIVFSGYHFGEQHWVRR
MGRRIWLQKFLYTSYGLTILMLLFAAHAEQVAEVIFDITGFSVQPAWFTYGCLGFLG
LFLIICFFNFFWTQFKRYVFRELFLLLVFFIVENTASLIWAFAIYFVVWHSVPSLADQIR
LLYGSVNLKNGSKYMRSSAIYWVGALVTLALAFLYFRESSLGFMPLFFSFLAAITFPH
VLVISRLYRS
SEQ ID NO: 78 (Sphingomonadales bacterium) (MBM3922853.1)
MLPILKQQSWLTLTGLIMGLIFWLSPIPQSWQWIFFLLVIISVGLPHGALDFLVAGKGR
NNAKLGRFLLFYLSQSVAFLLLWFWPLAAVSVFIVLSAYHFGETDLQLFNPAPLRKHI
AIICYGMATLLALLFSHFTELTALIPEIAQFVQQFKILQWLQEYYLPIYAGLMGGALLL
LASEKNIDRIKLASLLLLCIPQLLIFYMPFLAGFTYYFGIWHSLLSIVTIQNNTNTTGWA
NIRKLINPQTILFALLALALMGLLYWGISSFFNYDNQLLIFFIGLSILAIPHMQLMHELL
SRRFS
SEQ ID NO: 79 (Ferruginibacter sp.) (MBC7922457.1)
MRYVHSTLFHVQWLLQVLLTGLFCLSDWGSTTQAVLCGALLCTVGIPHGANDHAY
RPNTTSVGLLGFLGVYLGSMGAYLALWWSAPALALLLFFAISLHHFGQSNFENRSV
WYLPSLLWGAWVLVFPVLLHWDEAMGIFGAMVGSGVSWEKSASGLSGMWRGLAA
LLLALFYLLALLRLEREQWTRYLLQWGVVSVWYWLTPLLFGFIVVFCVWHSLQSLQ
HQLRHFQSAGGGSRRQFFLGLLPFGLLALAGFGGYVYFRGFAVDEAFVLLSLITLPH
VVVMHRLYGAPASPTSPTTASGKRAV
SEQ ID NO: 80 (Actinobacteria bacterium) (MTB30923.1)
MESAKLKVFSRVRTFSSLMVALAIALSILFSSWLGAESLNWQVVMAVIALAIGIPHGA
LDHLVTLPKSNPVKMSIFVFIYVAIALVAIWAILQWNVWGFIAVVIMSSAHFGVGDS
AFISEYDDLRGSSSPLPVWAYAPAAGLLPVVIPLVNSRSTEALSKVNIALVDWHHGFT
SEIQIIVAIITTLSVITLISRKRFRDLLDVSLLALLASVAPPLVAFAVYFGCWHAMRHTA
RLTSLLPRSQSSYEKAHARKAFISAVVPGLPALAGTLIFVALLAGFSHQNVSDNFLWL
TLVTIWALTVPHMIVTAKLDRAALKN
SEQ ID NO: 81 (Spirosoma fluviale) (WP_097126042.1)
MITSLLTKLSSYIQRTPTGLTILAGTLLACYQYWVGPLPIWLQLVFCISLLLLVGIPHG
ALDHLIEQERTIRQAQPFSLARFLTKYVLMIAIYGLAWVFFPVFSLTIFLLISAWHFGE
TDLENAPDDRYWSLIRLVAGGFVVAFILLTHAPEVTPILGRIIQNNPQALNLWGEAAA
GAGSLLRGWATLAVVLAMLALGHQPMRLNGWRLARLGMVLLLTYMLPLLPAFML
YFGGWHSLSSFGIIQSYLQRPGKPTQTIWKLWRQSIPLTLLAFGFLMAGAGIWQSYIP
LFDPLPYLFILLSTITLPHIQVMHQLDLLRK
SEQ ID NO: 82 (Actinobacteria bacterium) (MBC7464309.1)
MEDALPKIFSWVEWWSRAAILTTVILIIFLRIIHIELSLSVQIIMALVGLIIGIPHGAIDHL
VSIPSQPRVRFYTYIVAYIAIALMAGWAISTWNMFGFRVVVIMSALHFGFGDAAYRN
EWKSATNSRKYPWFIETAYALPAGFMPVVLPLTDPRSLSALNRIHPGLKDWAGANV
HLFRGITLVMFICALVVLLIAKSFSFALDLGLLGLLTLIAPPLVSFAIYFGFWHAARHT
VRLIPKLEKARALALNGDGLKALRGAITPGLYAVAGTALLASGLTLVTKEISSSGFLW
STLVIIWGLTVPHMLTTSSFDFQAIRK
SEQ ID NO: 83 (Marinobacter psychrophilus) (WP_048386795.1)
MVINDTANTENVLYRSSGGRRHQFIILMIASLALIVGFMLPVLSIEQQFWIVLLPIGVF
GLSHGGADPMILKQLTATSPTGLFVVLCAYLMASLAFVALIWVLPVLALLVFLGLSI
WHFGYTDEAFLSSAKNPPLRWLFGSTPILGPMLGHPQQTSELFAWLIKNESQVVLDIV
VWLGPCLAVVWLFGFGCLLFGRLNRPGPRVLVELCLVGAALIALPPLLGFAFYFCAI
HSVRHFLSIAEHRLLSNQKSLFQAFPVRKILPATLGAIAMACVAWGVIVLIGPATSLM
ADAVRVMFWALAALTLPHAIIVRLWWNQRLVE
SEQ ID NO: 84 (Actinomycetales bacterium) (MBU6347572.1)
MSESQLRLLNRTRELSFFLILSGLVVAFFASRMFGDNADWQIALALAALTIGIPHGAV
DHLLSVPKFFSLRMAIFLFGYLLVAGMAAWFILLWPLLGFQLIVLISALHFGVGDASF
YMELTGRLRKPGFPRIVYALAAGCTPVLIPLTNSQTKAALQNVNPKLIDWAGPFAKQ
IFILCIALNLATAALLAIKKFYWPTLDLLLLLALSLITPPLVAFALYFGFWHAFRHTTR
LTLEYRPALQLHVPDQPWRSFWKVVRAGLPAVMVVLIFTAWLKFTNGTSTSEDFLW
YLLVVTWALTVPHMALTAKSDAVALKLKPSRIREST
SEQ ID NO: 85 (Geodermatophilus chilensis) (WP_100501915.1)
MASPALGRRGASAPAPEGLPPGQALARPVRAATGVSLGAAGAACLVELTLPGGWG
DAAPVVLVAGLLLGLPHGAVDHLVPGFRSGRRPAVLAGFAAGYAVLAAVGWLAFR
AAPGAALLAFVAVSVWHFGSGETAFADLRAGHPVRRRLAAALVVGALVLLVPLVR
GAEETAAVVAAVVPGSDGRLPPELTAVVLGAVLPATAALAASLLAARRWLDAAEL
GVLTALVLVVPPLAAFGVWFGCWHAVRHVARVVAEDPGARADLVAGRLARPLRRF
AVQAALPTAAVLVVLALLWEAAGGWRAFVATDVPVLAALTLPHVLVVGWLDRLE
STGTGPGGRTSGGP
SEQ ID NO: 86 (Geodermatophilus sp. TF02-6) (RBY79850.1)
MARRCYRGRRRRQRGATGLTVPSATVRGRAAVTAPSPGRRSATPFAAPARTATVVS
TTAAVAVLLAEVAVPGGWGDAAWGVLVGGLLLGLPHGAVDHLVPGRRLGWRPVR
LAVFAAGYAALATVAWLVFRAWPGPALVAFVAVSAWHFGTGETAFADLRAGRPV
GRRPIAAAVVGAVVLLVPLVRGSADTAAVVAAVVPGSAGRLPAWLPATVLGVVLP
AAAVLAARLVGGRRWVEAAELVLLACLGLVVPPLAAFGVYFGCWHSVRHVARVV
AEDPAGAGDLAAGRLGRPLRRFAVQAALPTAAVLAVLALLWSAADGWPSFVATDL
PVLAAVTVPHALVVAWLDRAPS
SEQ ID NO: 87 (Nocardioides sp. TRM66260-LWL) (WP_224127607.1)
MLRPVPAPVPASSPLRALRAGVIGSRRAALVGVGVGALLTGAAAVGLASGPAEALT
WSLVGLGLLVGLPHGSIDHVLAARMTGWSLPVAAAAYGAVAAATWGLLHAPHGV
GAATLVGVLALSLLHFGLGELEVFRADAAWHPAPAVAAALALAGTGALLVPLARA
GDDLVGVATSISPTLGAVLSSPAARLGLIAVWGAALLVAVAAAWRAGRSDVARDLL
LVAALGTLAPPLLAFAWWFGGWHGVRHLARLLGAEPGSARLLEREGVGPAARHLA
RIAAAPSLAAAATVVALLVLTARAGDPTAALGGTLVTLLALTVPHMAVVLWMDLR
TWRTERTRRPAAGRALRPR
SEQ ID NO: 88 (Aspergillus pseudotamarii) (XP_031915663.1)
MTGKEKSHKHPYFSGNYAPIYTVQHAHPCEVQGTVPEEFLGGQYVRNGSNSLQDDD
RRDLHWFDGDGMLSGVFFKRVPGSKVQQPLYSNRYILTDVHCATAAYPHINPILSSA
TTLLSPMVSPLKVFMGMLRTMALMLSSFLGFVDRPIRRISTANTNILYHDGRVLATM
ETGPPMRVYLPSLNTVGWFTGGSAEGEATDQIMEPSIGDPGIERFHNEMTTAHPHCD
YQTGELLLFHSTFIFPFVHYSIISPGWNGKHGSYLNQPVPGFTSGKMIHDFGVSRKHTI
MLDIPLSMDPTNITRNKPAIQYDPHGRTRFGVFPRYCPAQIQWYETDPCCIFHTVNSW
DDSVPWGTRVHMLFPADRSGAITEQWALSAIPFEFPHVPQHLEMTAAQFVYGCSMS
EGNFATRQKSSVKVDCLVKIDVQRLLQAAEVQPPTQITGCVDQRSINEILATNDVND
PIQVFALPYGWYAQECSFVPRKDGTSEEDGWLVTYVFDESQLDSNGNAPATSRSEL
WIIDARNMRDVVARVLLPQRVPYGMHGDWFSEEQILNQREVAEFRSLD
SEQ ID NO: 89 (Ilyonectria sp. MPI-CAGE-AT-0026) (KAH6971494.1)
MTSTKSPKTHPYRSGNFAPVRNQMSMEICEVQGTIPDELIGGQYVHNSSNPLAYDDD
PSRPMHWFDGHGMLSAIYFSRQDDNNTLDIQAMYSNQYVLTDIYLAESNSRVRAPIL
PSLVALLSPWSSVFTILRGYFRMIWMVLCSWVGHSLIPIRRISVANVNVVYHDGRFLA
LCETGPPMRVTLPDLKTVGWFNGCMAEGEFPEDSKTVKRSGFAGDGLLSFFREWTT
AHPHIDPLTGEMMLLHPTFIRPYTRYSVLAGSSSNAGNGRQIFGKSVPEVLSPKIMHD
FGVSRRNTVIIDMPLSLDPFGLAFGESAIAYNSKGQTRFGIFPRHVPELVQWFATEPCC
IFHTVNTWEDTVKGETAVSMLVCRMTGPSMVYATGNLPLPTSQGHREEECRLYYYQ
FSVGGPHRAITEQWALSAIPFEVPHVPKHLTMSATRFVYGCSVAGGNYADHLENAL
KIGSFVKFDVQKLIARGLANHPTSVSGCVDNRSVEDILASDDPDDPIQIFPMPKGIYGQ
ECVFVPRSGGKSEDDGWLLTLVFDESQLDVDGNAPDDCKSELWVIDAKTMKGIVVR
IKLPQRVPYGFHGNWFSEHEIAQQHPVKKFRSQK
SEQ ID NO: 90 (Ophiocordyceps camponoti-rufipedis) (PHH79717.1)
MAHHPYRTGNYAPCRRESHLAGCFVRLGSVPDDLAGGQYVRNGSNPLGYEGDATG
RDMHWFDGHGMLSAVYFSVPEKPGATIMPLFSNRFVRTDVFLAEAAAASPLRVPLL
PSIATMLQPSLRMYLRLARCILSLLLTVLLSLIPGSRRQSIRRISVANTHIVRHGGRFLA
TCESGPPIRIRLPELATVGWFNGLGAEGEPRRGVWPGGHGWRAGLLGFLREWTTAH
PHVDPQTGEMVLFHSTFVRPYVRYSVLAGAPHPDHGPTPSRRLPFNSPVPGVRSARM
MHDLGVSRRHTVIIDMPLSLDPFQLLRGRPVVAFDRTSPTRFGVFPRHEPAKVRWLV
SAASCCIFHTVNTWDDPAAVHMLVCRMGEPSIVYATGGLPTPTTDMDDATLTARNH
CRLFYYQFRLDQDPAVAQQWALSAIPFEFPHVPGHVAMSAARFVYGCSVRGGTYAE
QLANTMKIGSLVKIDVAVLIARGLKQPPAPTWGCVDERTVDEILADDDDDDAIRIFV
MPEGHYAQECVFVPRKGGCREDDGWLLTFVFDEAQLDDFGNAPDDARSELWIIDAR
SMRDVVARVVLPQRVPYGLHGSWFPREELENQRPVATFRESKA
SEQ ID NO: 91 (Penicillium sp. RFL-2021a) (KAF7719097.1)
MTPWFLSGNFEPIQRQQELTSCPYSGTIPEELAGGQYVRNGGNPQQMPKETERSHW
WDGDGMLAGVYFNQSDDHDSKLQPQFVNQYLLTDVYLAAKSMSSVFSPVVLSLSV
MLDPCISIFDFLCELIRCAVLVLWSHISPMVPAIRNLSVANTSLSFHDGRALAHCQSGP
PLRVLLPTLQTVGWFNGASAEGEPETHSSFEEVIGGRGPLAFLKNWTTSHPKVDPDS
QQMVTFQSSFLPPFVWYSVLPDSGHDSGKSSLSQTKLINQPILGVTSPKLMHDFGVSG
SHTIILDLPLSLNPLNTIKGKPPVQYNPQSPSRFGIFPRLQPQNIRWYETDACLIFHTAN
TWDGVEISPYSKESATVVNMLACRTTTPAVLYTSSSSSMSLGAEFFEQGHDACRLYY
YSFNISDPINNQIMHEWALSAIPFEFPTVSPKYEMRNARFVYGCSTTDSKSSASIGQGV
KVDCLVKMDVQKLIQKGRKESPTPIVGCVDTRSIDEVIASKIMDDPIQVFQAPKGWY
TQEASFVPRQYATREDDGFLLAFMFDESQLHHDRSCPLNARSELWVIDALDMETVV
TRILLPQRVPYGFHGHWFSREDIERQRAVASLRRGTM
SEQ ID NO: 92 (Ramaria rubella) (KAF8591152.1)
MKTHPYLAGNFSPIHTEKDLTPCQFVGSIPKELWGGQYVRNGGNPAHSTALGRDYH
WFDGDGMLTGVLFKRIRSQVEPCFVNKFLLTDVYLSGRRSGALFLSSPLLPSISTLVSP
TVSLHVLLKAIFRALFLFIKNSGIRLSVANTNILFHDQRALATCESGPPLEVTLPELDTV
DWWTFGNRISGHTKERRGLGGERGLKAFFEEWTTGHPRVDPITSELILFGSSFLPPYV
RYTVISSSGTALPSLLGAPVPIRSPKMMHDFGVSLQHTVILDLPLTLDPLNIARGKPIV
EFYPEGRSRFGVFPRYHPGLVKWFESSACAIFHTANTWSDETAVNLLACRFASAKLV
YIAGDISLPPSCIDSEDVCQLYYYRFPLSPHDATPSISHEFALSAIPYEFPSISHSVSMQD
AQYVYGCTMRSGSFDAALGAAKIDCLVRVDVRTLIRRGIDSGICHGGVVDSRQVVEI
LASTDEVDPIKIFEMPQGWYAQECSFVSKQEAKSEADGYLLAYVFDESQLNDDGSAP
NCARSELWILNAETMNEVIGRVKLPQRVPYGLHGNFFSEVQIASQRPSRPRRTISLHN
NGVNTFLPTFLCLAMVCNIYVFSAMLGEASNLT
SEQ ID NO: 93 (Aspergillus alliaceus) (KAE8385184.1)
MNISGTGVEGNSRKHRYMSGNFAPVSETHSSEPCYCIGSVPEELSGGQYVRNGGNPA
IEVEIAGKTHWYDGDGMLSGVYFRSSPGGAHIVAEYSNQYVMTDVLLATMDHPTLD
EPIPLSIATLLDPHVTLGQFLKRLWRMSKVIATSYLEFYSHTIKRISVANTNIVYHDGR
ALALCESGPPLRVSLPDLRTVGWFNGLISEGEPMEDTDTSGFGASGFFRLFQEMTTA
HPRCDPQTQEMIIFHSVFIPPYVSYSVIPKRGKTSSRPANMLLNVPVPGLSSPKVMHDF
GVSRKYTVMIDLPLTVKWSNLWHTKPVVSFDVSSKAAFGVFPRHNPDQIRWFHTEA
CCIFHTVNTWDTEPTGDEKGLQPRVNMLVCRMNSPAILYTTADLDPPVVPAQDGIPQ
CRLYFYQFDLTRPWNIISHQWALSAIPFEFPHVPKQSTMSETRYVYGCSTTEGCFGTA
LGKAVKIDCIVKVDAKGLIQQGLETSPEPIFGCVDCRSVQEIVQDRKSNDPISIFRLPT
GWYAQECSFVPRQGTVDEDDGWLLSFVFDESQLDSAGEAMSNARSELWIIDARDM
QTVAARVVLPQRVPYGMHGNWFSREEIEDQQPVESYRCLPKKVGHS
SEQ ID NO: 94 (Peniophora sp. CBMAI 1063) (VDB91879.1)
MNHAYLSGNRAPVTNEVPLTPCRILQGTIPPQLSGGIYVRNGSNPAPNVNTDNLRPY
HMFDGDGMLAGVYFDFERGPLFTSRWLQTDVLAAAKRFSLSRATFPSITSLIDARAP
QLLVLLEYLRCILVVALSWILALWNKAGGGIARISVANTAIIWWDRRALATCESGPP
MRVGLPQLDTRGWWLLGGALPRVSMLQSIFKLKAFFQEWMTAHPHVDPETNEFVA
FHASFFAPYLYYTVLQPSNSSSRSQLVRKPVPGLRAPRMMHDFGVTPTHTLFLDFPLS
LDILPSIKRQSISPSLTYDPTIPSRIGVLPRYAPEEVVWFELDRPGGCVFHTTNAWDAP
EQRAVEMLVSRMGGPALVYAAGALPVPTHAGTDECLLYYYRLPLHDTLAAQRHPR
PSHAFPLLSLPFEFSAIHPARSMHSQRWVWGCSFFDHPVPGPWDPYTSTSHHGPETAR
LPPAELTGYSDVLSSGFKVDTLLKIDTSRLITQGLERGEGEHAVPVDNRSAQELWDA
QEAGDDTSVRMLRLPEGCFAQEVTFVPRHEVLPEELQDEEDDGWLLTYVFDERQLN
EAEKGGSCGLESAISELWIIDARTMDIGTVCRIALPQRVPYGLHGEWFSAGRIAAQEP
SEQ ID NO: 95 (Rhodotorula sp. CCFEE 5036) (TKA55938.1)
MGGATNPHPYKTGNFTPIREERILERCEWTGDIPEELVGGMYVRNGGEPALAELQNG
SGDGPAYHWFDGDGMLSGVFFEKASSSTSDPKQRVQPTFINRYVLTDVFLASKELGV
RDPILPSIATLLGGVTSLHLVLFAIFRAVFLAFCSFFTKSPLRHLSVANTSLLWHDGRA
LATCESGPMTWVTLPELDTVGYYSLEGENGETGLREGMLGWMKEWTTAHPKYDPH
TGELMLFHMTFLPPYLSYSVVPSTHTPTPSEKKVTPRLLAAPVPISAPRMMHDMAAS
RTHSILLDTPLSLDPRNLALGKPVIGYEPASPTRFGVFPRHAPNLLRWYLAPACIIFHT
AFAYDEYTANSPESLAAVNLVCCRLNSPRLVYSAGNLMLPKSQELPAGAKEACELY
YYRFPTSNSASQNLEPSHQFPLASIPFEFPTVPQDRVVGPAKYVYGCSVKHGNFDAAL
GGAAKIDCLVKINATALIQRGIKRAETGKQDAETPVDQRTIAEVLSSQTPRGGSKSSS
SATEIADSDDVPIRLFELPPLHYAQEPSFVPRSQPRAEDDGFLLTYVFDERQVDLVSG
RPFKDARSELWVIDAWDMRTVVARVKLPQRVPYGLHGHWFAADDLQHQRSPPSIR
TRPTATS
SEQ ID NO: 96 (Acaromyces ingoldii) (XP_025380094.1)
MRHPYTSGNYTPVKRELPLTEGVVVQGSVPTCFHGGQYVRNGGNPLADGDEKRDA
HWFDGDGMLTGVLFSEPTPGEKEARSRDARPMFINKYILTDVLLSTSTKYRQPILPSIT
SFALPFQWSALPYLLAMLLRSIVLVFLSWLPFQRRPRVGRISVANTSVYWHDGRAFA
GCESGPPIQILLPGLETADWWHGGWAKGQGPFKMLNEFTTAHPRVDPVTNELLLYH
MSFAAPFLRVSVIPPRSSRKPALLGAPVRGVARPKLAHDFGATATKTILIDLPLVLDPR
NLVAGQSMLSYHRDMPTRFGVMPRWAPQEIQWYESESCSIFHAANAWDDDEDAAC
LLACRLNSATLVYSAGNIATPDNCKPPLGQEERCHLYFWRFAPRSNTISHEFALCNIP
LEFPTINERFSQRKNRFVYGASMTCGSFDAGLGSRNAKIDCLAKVDVEALVCRGRSR
QDAGLLSKGECVDDRTVSEILQSGEKEDAIKIFALPPLCYGQEATFVPRVEGGDEDDG
YLVFFVFDESKGLNADGDCTPDAKSELYIIDAKDMKTVVCRIELPQRVPYGLHGHFF
SREDIASQIPIDASKVRSWALARKGLGPGPGLGNGGKPALAKAPSNFLVGALEAWSD
ALRGTIEGILA
SEQ ID NO: 97 (Acremonium chrysogenum ATCC 11550) (KFH47490.1)
MDHQHPYLSGNFAPVQSSLPLTACPAEGKIPSDLAGGQYIRNGANPVTPNEHLDRDT
HWLDGDGMLSGVYFRRAGDKGTEIEPQFVNRYLQTDVYRYARHSRFLTRPVLPSLT
TTLLLGGGPVVRLVRVLRAVLRTLILAVVSRLPGRPSIRKMSVANTSVVYHDGRALA
TGGAGPPLRFLLPGLETVGWFNGRRAQNEPRGDGRSGFRGDGPLAVLRGWTTTHPR
VDPVTRELIVLRSTACKPYVRYSIVKPSKQTEPGSSIFDEPVPGVTTPKMVHDFGVSS
HHTVIMDLPLELNPKNLLKGTAAVRYDLTSRSRFGVFPRYAPYGIRWFETSPCVIFRT
ANCWDTISSATKERPAETLVNMLVCRRTSATAVYNTGNITAPWRGPVPPEYAEEEQC
RLYYYSFPITDASHTERHIRHQWALSAIPFDFPIIPADKSMSECRYVYGCSSSHSSTPAP
GSAAKMDYLVKMDAKSLVACGIADPPPAVTGCVDTRTLEQVWAESSNGDDDDDD
DPIKLFRMPRGWYAQEPMFVARADTESEDDGWLLTYVFDESTGLDEEWECREGAK
GELWIIDARNMRDVAARVKLPQRVPYGLHGSWFSEGEILAQRPHGSVRDAGAVVSK
RTVWKWEGILGGLGSVVERLVG
SEQ ID NO: 98 (Sporormia fimetaria CBS 119925) (KAF2741777.1)
MTPKKPSHPYLAGNFAPIHSTHASTQCSFTGTIPKELLGGQYVRNGGNPITDQDLARD
AHWFDGDGMLTGVHFVRSPSNPGEALPFFTNQYILTDLCIFAHQNRNLRTPLLPSIAT
LVDPASSLFTILFRLLRAVFLIFVSHLQRPGKIRVKRNSVANTGIHFHDGRALASCESG
PPMRISLPGLETVGWFDGANCEGELQSNGEKKEDTFRGTGLLGFMREWTTGHPKVD
PVTGEMVLFHSSFKPPYVHYSVLPQNRAATAERLPKLLVVPVPGVSGSKLMHDFGV
AREHTVILDLPLTLSAFNMMKRQPVLYYDCDKPARFGVFPRRQPEKVRWFETAGCCI
FHIANTWDERDANGNIQAVKMLACRQTSASVVFNTGNITIPEMPRSRGRRLQNLARR
GSDEEEGTPDDKTPLLDSSTRSSAGDTEQCRLYYYRFSLDRPGDVITHQYALTRVPFE
FPTLNPAYDMSAARYIYGCSTTEDTFGTALGKATKIDVLLKVDVTTLMERGAAYPPR
SVTGCVDMRSMAELFASKDQQDPIRTFLLAPHHFAQEARFVSRENPVAEDDGYLLF
YVFDERQLDGKGECLPNAMSELWILDARNMKDVVVRVRLPQRVPYGLHGNWFSEE
QIQTQRDIEGFRSLPVDPEKGVMARLRSLLVGAVG
SEQ ID NO: 99 (Tilletiopsis washingtonensis) (XP_025600717.1)
MASSSAHPYLAGNYAPVKRELPLTPCTYTGAIPHELVGGQYVRNGGNPSADADGER
DAHWFDGDGMLTGVLFARRHDGSVQPEFINRYVLTDVLMATSASATRPILPSIATLS
SPHVTLFRVLLHVVRALWLCLSTFLPWHPAKLAVQRISVANTSIWWHDGKAYAGCE
SGPPMRILLPGMETAGWWTGAEHDPQSARGWSKAGLAGLFAEMTTAHPHVDPVSG
ELLLYHMSFFEPFLRVTVIPPRQRAEKAPVCPAPIIGRAVPGLAQPKQMHDFGVSASH
TLILDLPLSLDPRNIVRGRPILHYDHLGPTRYGIFPRHAPEAVRWFEEAQACLIFHTAN
AWDDDAAATVSLLACRLNSATLIYSAGHTVPPPHALPLNGAPESCRLHYWRFTTDPS
DASLRPQASFALSAIPFEFPTMNQAYSTRRADFIYGASMRSGSFDAGLGRKSAKIDCL
AKIDAGTLLARAAKMSEAGETLGVDDVVDGRTVQELIAAQEAGEGADSAVRIFALP
EHHYAQEATFVPRANATAEDDGWLVFFVFDESAGLDADGVALPNAQSELWVLEAR
GMQDVVARIRLPQRVPYGLHGHFFDAAEIAAQEPVPHEAVRTWARAATGGAASSAI
VPAAAASTAVVAAAASAAVVSAAASPAFVAAAAPSALVPVST
SEQ ID NO: 100 (Diaporthe batatas) (XP_044637864.1)
MSSESEHLIPLAESSPRHPYLSGNFAPIKKTRSLTPCVYSGSIPLDLGGGQYVRNGGNP
VSNDDLGRDAHWFDGDGMLSGVLFRRTGEKGSTIQPEFVNQFLLTDVYAYARRRA
GPAGELDRSLSRPVLPSIATLVNPALGFFTILWAIVRTIALVLLSHLSRSRYPIKKISVA
NTSVMYHDGRALALCESGPPLRFALPGLETIGWFNGRTAENEPRNQDDDQKGGFGG
GGALSFMREWTTAHPRVDPVTKELIAFHAVFIKPFCYYSIVPPSKRKESLLQPPLSARL
SMPVPGVRAPKMMHDFGASRNHTVILDLPLTLDPMNLARGVPVLSYDSRGKSRYGV
FPRYRPDLVQWFETNPCVIFHTANCWDSVTHTGEMNGANIQQTSVNLLACRLTSASL
VFSAGNLPTPAVKPVPPEYAEEEQCRLYYYNFPLSANDGTDEPGHRIRHQWALSAISF
EFPTLSPNHSMSEARYIYGCSTGHASYTVALGKAAKIDYLAKIDVKTLIERGVQHPPQ
PIRGCVDTRTVVEVQESHDPNDPIKLFKMPDGWFAQEPRFVPRREIERQSEDDGWLL
TYVFDESQLGSDGECGEDAVSELWIIDAKTLKDVVARVKLPQRVPYGLHGSWFSED
EINEQRPFTSLRSVADQEGSAFSRSLQGMVERWIG
SEQ ID NO: 101 (Taphrina deformans PYCC 5710) (CCG82935.1)
MEELAISRRLTDNKVHLAAKSTRSRHPYLSGNYWPQRNELHLSPCEVIGSIPQSLAGG
QFVRNGPNSSIPPEDGQNYHMFDGDGMIHGVYFKSIPGKDELESLYINRFVHTDVYL
ASQYWGTSLLPSIASLIDPLINTVQVLKSILRSGAIAFLSGSARLSTANTALVYHDNRL
LATCESGPPMHIGLPELETVDWHTFTDERTGVSLAPIPGSSKEWITGHPKVDPVSGEL
LMFGYDIFQLFNPHIRFSVIDADGSHKNFQQPVYLKSKTPKMMHDFAITQTRAVILDL
PLTMSPFNIVGGGKPMLHFDISLTSKFGVLPRTYDAKRDRDQVVWFESDSCMIFHTV
NAWDDHDSAGNVVGVNLVACRFKGAKLVFAAGAIQMPSAENLPSDVVHLYYYRF
DLNSGKISHEYPLSDYPIEYPSINTRHLSLPNRYAYGASMREGSFDTALSGAKVDVLV
KCDIQALIRSPPTNVTQSSSVTPPSGAHVKTILLPPGLYASEATFVPNTSETATGKEDD
GYILVHIFDESTRDPVTKDALDDKGSEVWIISAESFSLPPICKVRLPQRVPYGLHGLW
VTKSQIESQRDAKRNSEGDIVAAKASREHTSVRIITNFWKWSLVWAGRSTVGGLSSG
RYAASLFIGWTVFILLTYRLFGFIVQHTMQSLSR
SEQ ID NO: 102 (Stereum hirsutum FP-91666 SS1) (XP_007305246.1)
MKDDSKKSHPYLTGNYSPVRKEWPLTSCQYMGTIPPELYGGQYVRNGANPFHVDK
DSWDLNPRDYHWFDGDGMLTGVYFQRLPSTSEASNAQTISPHFVNAYVLTDLYLHA
RTSPYLRRPLLPSISSFLNPLAGTLGVILSVLRAVFIVVLSHISAFLSRQQSYSVTKISVA
NTSIVYHDGRALATCESGPPMRVLLPSLTTVGWFDGTHGDGDDVRVNEDPSFGAKP
GLLGWMDQWTTGHPRVDPCTHEMILYHSTVLPPYISYTIIPPSSPSTIPFQPAHPRLRN
APIPGVHSPKLSHDFACTRTHTIILDLPLYLDPLNLLSSFLPSRKKNQVMHYQDDEFA
RFGVFPRYEPHKVRWFEDVEGGACCIFHTVCAWDEMDPRGERVVAVNLLACRLDS
AALVYSAGNLATPRTPRDDSTSGLAVDDKEGKCELYYYRFDLSDPDANNITHAFPLS
TIPFEFPVVPPSAGMGSATTSSRRFRSPRFVYGCSMQHGTFGAALGGSNARIDAIAKV
DVHDLIERGAHRHYDASKSRPAIDQRTVQEILGTADTSVDKGAITIFTFPPGHCGSEPS
FCPRANAISEDDGYLIFYVFDERQLERGLSLDEARSELWVVDARNMKDVICRVELPQ
RVPYGLHAHFFTEDEILGQRGVHSLRSLPAGVEKNDTAGL
SEQ ID NO: 103 (Pseudocercospora fijiensis CIRAD86) (XP_007924069.1)
MSTLGKRKRSPSTNIERTPQPKHPYLSGNFAPIQHTLPLTPCTYTGRIPEELADGEYVR
NGSNPVSNSDLGRDAHWFDGDGMLSGVSFSKKEDTGEIQPEFVNQFILTDLYLSAKS
NGRLRVPILPSIATLVNPLASLMFVTVRILRTILLVILSHLPGSRQKIKKISVANTSVVY
HDGRAMAFCESGPPMRIQLPGLETVGWFNGAVAEGEPANESKEKEAVLGENSGLISF
MREWTTAHPKVDPQTKEMLMFHSSFAPPYVQYSIIPQTVRAAAEVSPPTRKMLNAG
VPGIKSAKMMHDFGVSSAHTIIMDLPLSLDPLNQLKGLPPVVYNPDRPSRFGVFPRRH
PKQTKWFETSACCIFHTANTWDEVGLDGSTTSVSMLACRLTSATLIYAAGNMAPPPL
LRVPSLEEHLLKDPFAEEQCRLYYYNFDLDSGSISHQWALSAVPFEFPSVRPDLEMSQ
ARFVYGCSTTTTDFGSALGKATKIDALVKIDATKLIERGRAHPPKSVTGVVDGRTVS
QVLISRDPRDPIKVFRMPEGWFAQEPRFVPRERSAHERQLNDEPLDEDDGYLLFYAF
DETQLLANGEVPDDSAKDQRAKSELWIVNAKNMRDVIARVHLPQRVPYGLHGSWF
SAEQISEQRPVDTIRSMEEVMKEKNDNGLWMKVRERIETLLG
SEQ ID NO: 104 (Meira miltonrushii) (XP_025354342.1)
MTGSSSSSTKSRSSSSASSETSIIPFPKKTLHPYLTGNFAPIKRELPLTQCKVAYGKVPE
EVCGGQYIRNGGNPFKDEDQDRQAHWFDGDGMLTGVLFQSDGEGIRPHFINRYILT
DVLLSTSQASRKPFLPSISTFASQLSFFGFFILMGEIMRTFVLAMLSFLPFQSRPSVKRIS
AANTSIWWHDGQAFAGCESGPPIRVLLPGLETAAWWTGEEETEGGWSKGGMGPIK
MLKEFTTAHPRIDPETDELLLYHMSFAAPYLRISVVPTRKSIEAGAKPLMGAAVPGLN
KPKLAHDFCATSKRTILIDMPLVLDPRNLLQGKPMLSYEENMPTRFGILPRRSPEKVK
WYESEGCCIYHAANAWDDEATSSTNLVACRLNSATLVYAAGNLDTPSHAKATCGT
EKCQLYYWQFADAPEDNQAVPEKNEKAQVLREFALSDVPVEFPTMNDSYSQQCNR
FVYGASMTTGSFDAGLGSRSAKIDCLAKFDIQTLIAKGKKAMDAGQLQKGEAVDKR
TVQDVLAAEQTEDDPIRLFALPPHHYAQEATFVPRLNAKSEDDGFLLFFVFDESTGL
DYDGDALPNAISQLWILDAKTMKDVICKICLPQRVPYGLHGHFFSATEIQNQRPVDT
DQVRNWALAQTAQTGIGLGGVKASSDHDNNLSSFTAFAYSAVANFRNSIEHFLG
SEQ ID NO: 105 (Kwoniella heveanensis CBS 569) (OCF41229.1)
MTKTLRAVPDGGHIHPFLKGNFAPVTDEYISHPCEVLEGEIPDELLGGQYIRNGGNPV
YPPEQGRHYHWFDGDGMLHGVYFPAEKGAEPLYTNRHLATPLMTMTLLLLRSPIPSI
ALLISPLSSLHRIVMAIVQSFFIAVRARMGVLSVANTSDAVRSQRLLATCESGPPLEVR
VPDLQTVDWDRLEEESTGENLGQRRGQWEWWKRIGLSRVQEDWMTAHPRIDPVDG
SLIFYSTQMFDAPHVRYSVIDRKGRHVVWKEGVDVGRAKMMHDFAATRTHTVLLN
LPLTLAPHNLFQRHPVPLIHFDRSLPSEFVILPRLYRSHDTPQHPIRFRESEPSLIFHTAN
AWDEYDREGDLVAINMLGCRFKSAKLVYAAGQVDVPLEEQKFGEDDVVRLHYHQF
DMRGYTTSAEPPTEGEITYSFPLSAMPFEFPCTPTHLGMSAARYVYGCTMKSGSFDE
RLGGAAKVDCIAKLDVLELISRGQRRGKGKNILPVDERSSAQILEDAQRGIKGPIQIFA
FPEGCYAQETQFIPRSDPKSEDDGFLVTYVYDEKYLQADGTPSPALGAGSELWIIDAK
RLAEGTSGVVAKVKLPQRVPYGLHGTFVPASLIRNQRVLPQPLADQPLLQDKLARSR
LQYFVSILFDRPATRKRSHFERGVLWVLWPAAVLMSFLSLWTASKHLFWLR
SEQ ID NO: 106 (Epichloe festucae Fl1) (QPG95396.1)
MYHPSEALSSVSAVTLALFICVTITSSILTWTWLNKDKDKKERTVPHPYLQGNFAPIQ
TTRPLTPCKYTGTLPRELRGGQYVRNGGNPVTNDHLGRDAHWFDGDGMLSGVAFIE
SDSDDAVVPHFVNQFILTDVYLSLSSMTTPLRTPILPSIATLIDPLSTLYTITSSILRTVSL
VVLSHLPGSLHPIKKISVANTGVLYHDGRALATCESGPPMRIALPTLDTIGWFNGHHA
EGEGQGQGQGEENHEPMDTGMEAGTGADAVVIGGTGPLSFMREWTTAHPRSDPVT
GELLLFHSTFIAPFVRYSVIPAEQDSRKTLPRPRLLNAAVPGISSPKMMHDFGVSPTHT
IIMDLPLSLAPQNLARNLPIVSYDPSSPSRFGIFPRYSPQTVRWFHTDPCCIFHTANTW
DSPDAVHLLACRLTSPSLIFSAGGIPVAPAKDDQCRLYYYQFPRDASSQSITHEWALL
SLPFEFPSVPDAMSMSPARYIYGCSSASNFSAALGRAVKIDALVRVDVQQLIARGIDD
PPAARVTGCVDTRSLDEIASSSDPNDPVRVFQLPAGWYAQEPRFVPRDGGSEEDGWL
LTYVFDESQMDEHGNVGVDAVSELWIIDAPRMSAGMRAVVARVRLPQRVPYGLHG
SWFSREEVDGQRPWNHSRSSELRETRKLGREVHEGLWGRARDVLISSCLA
SEQ ID NO: 107 (Pseudomicrostroma glucosiphilum) (XP_025348787.1)
MTRRTWALAQLNEKHGYLAGNYAPVEEETPLTPCMVVSGSIPNELAGGQYVRNGG
NPMANDDAGRESHWFDGDGMLSGVLFQRRANGSIAPHFVNRYVVTDILLATRQGA
HRPILPSIATLSSPSVSLLRVLYEVMRSLVLCLLTWIPLTWSIGLGKGTRRLSKISVANT
NIFYHDGKALAACESGPPMRIMLPKLETGGWWTGDEGKSWASGIGPLKIFNEMTTA
HPHTDTSTNELLLYHSTFVAPYLRISVIPPRSSRRSALIGATVPGMKEGKLMHDFAAT
KTRTVVMDLPLTLDPRNLLSGKPVVHYEPTAAVRFGVFPRREPEKVRWYEDPTACCI
YHTANCWDESQAETSQSGSSEDAVNFLACRLNSATLIYSAGNTLAPTTAKAPNGEEE
RCELHYWRFAEPPKATELLPPLDSTALEKAPKAQHDSPARITHSFPLSRVPFEFPTINL
AHAPAAGLGPNRFVYGCSMREGTFDAGMGRHSAKIDCLVKMDVQSLVKRGRARA
DAGKMKKGEAVDMRSMPEIIASAEHAQDGAVQGDITVFPLPHGWYAQETTFIPRSN
PQGEDDGFLLFFCFDELTHLHPATGEPLPSAVSQLWVLDARDMKTVVARIELPARVP
YGLHGKWFTEAEIDGQERVDEQSIRRWALRDVQKSDRRSKEERIKREGKLNVLNAL
RRWVELWA
SEQ ID NO: 108 (Fusarium tricinctum) (KAH7251169.1)
MSHSNEKTAQPLRHPYLIGNFAPIQKTTSLTPCTYSGRIPPELTGGQYVRNGGNPVSH
EDLGRDAHWFDGDGMLSGVAFRKMSPDGRVVPEFVNQYIFTDLYLSRKTTSVKSAI
MPSITTLVNPLSTMMRIVLATFRTMFLVLLSNLPGSQQSLKRTSVANTALVYHDGRA
LATCESGPPMRIQLPSLDTVGWYNGVEAEGESKDPTLNENDSPFGGDGLFKSMKEW
TTGHPKVDPISGEMILYHNTFLRPYVHYSVLPQRDEKTCSGSKLVNLPVPGVSGARM
MHDFGASRAHTIVMDLPLSLDPLNLFKGKEVVSYDSTKPSRFGVFPRHDPSSVRWFH
TAPCCIFHTANTWDTLSSDGNSSVNLLACRMTSSTVVYTAGNILPPVVTKTSGVRVK
GSKQDVDRWDVDGDAACRYEEAPLLESPGEKAGSADYFSSSDESDDYAQCRLYYY
EFDMSTKSQNKVKHQWALSAVPFEFPSVRPDRQMQQARYIYGCSTSASSFSVALGR
ADKIDLLVKMDAKTLIEKGKKTKTTSITGCVDRRTVSEIPYEQEDDDPVRIFRLPPNH
YAQEPRFIPRSSSTEEDAGYLLFYVFDESQILPSGDCPPSSVSELWVLDAQNMRDVVA
KVKLPQRVPYGLHGTWFSSSDIEEQRGVESFRNLEVMQKKKEGWVNGGFVKRSWM
TVREKLEKSVG
SEQ ID NO: 109 (Fusarium bulbicola) (KAF5980194.1)
MKFLQRYSFTQTSMPRPDKNISPPLRHPYLTGNFAPIHQTTNLTPCTYSGCIPSELTGG
QYVRNGGNPVSHQDHGKDAHWFDGDGMLSGVAFRKKSMDGRITPEFVNQYILTDL
YLSRKTTSIVSPIMPSITTLVNPLSTMFTIMFATFRTIFLVILSNLPGSQQAIKRISVANT
AVLYHDGRALATCESGPPMRIQLPSLDTVGWFDGVKAEGEPEKSQASSNDSPFGGSG
LFSFMKEWTTGHPKVDPVTGEMLLYHNTFMPPYVHYSVIPKSNEKASGHRLVNQSV
LGVSGARMMHDFGASRSHTIIMDLPLSLDPLNTMKGKEVVSYDPTKPSRFGVFPRHQ
PPSVRWYRTAPCCIFHTANTWDSHSSDQGSSVNLLACRMTSSTLVYTAGNIRPPVKS
KSTPARDWSDDKKETTCRYKEAPTLESPGVAAGLTDYFPTAESDDYDQCRLYYYEF
DTSTASQNEVKNQWALSSIPFEFPSVRPDREMQDARYIYGCSTSTSCFGVALGRADK
VDLLVKMDAKTLVRRGKKLNTTPVTGCVDRRSVSEILEQQKKDDPIKIFRLPPNHYA
QEPRFVPRASSSEEDDGYLLFYVFDESQLLPTGDCPPSAKSELWILDAKNMRDVVAK
VRLPQRVPYGLHGTWFSSRDIEGQRAVESLRSLEEVQRKKEDWVNGGGQIRKSWM
VLREKLERAVG
SEQ ID NO: 110 (Fusarium fujikuroi) (SCV49259.1)
MKFLQQNSFTQTSMSQPHEDVSPPLRHPYLTGNFAPIHKTTNLTPCTYSGCIPPELTG
GQYVRNGGNPVSHQDLGKDAHWFDGDGMLSGVAFRKASIDGKTIPEFVNQYILTDL
YLSRKTTSIASPIMPSITTLVNPLSTMFQIMFATFRTIFLVILSNLPGSQQAIKRISVANT
AVLYHDGRALATCESGPPMRIQLPSLDTVGWFDGVEAEGEPEISQAGSDDSPFGGSGI
FSFMKEWTTGHPKVDPVTGEMLLYHNTFMPPYVHCSVLPKSNEKAPGHRLVNQPVL
GVSGARMMHDFGASRSHTIIMDLPLSLDPLNTMKGKEVVAYDPTKPSRFGVFPRHLP
SSVRWFHTAPCCIFHTANTWDSQSSEGELSVNLLACRMTSSTLVYTAGNIRPPVRSRC
TQARVWSDEKEETACRYKEAPALESPGESTGLVDYFPITAESDDYDQCRLYYYEFDL
TMESRNHVKSQWALSAIPFEFPSVRPDREMQEARYIYGCSTSTSCFGVALGRADKVD
LLVKMDAKTLIQRGKKMNATSITGCVDRRSVCEILQEQRKDDPINIFRLPPNHYAQEP
RFVPRACSTDEDDGYLLFYVFDESQLLPSGDCPPSATSELWILDAKNMRDVVAKVRL
PQRVPYGLHGTWFSSQDIESQRSVKSLRSLEVVQRKKEEWVNGGGQIRKSWMVLRE
KLEKAVG
SEQ ID NO: 111 (Clavulina sp. PMI_390) (KAF8309329.1)
MKRTNIPHQKPTSVLPHPYLSGNFAPVSIELPLTECTVIGTIPSELAGGQYVRNGANPL
ANDDGTRDAHWFDGDGMLAGVFFCQDHSGAIRPHFVNRFVVTDVFAASAGRKTPL
AHPFVISISTMSSILRSPVSILIAILRTFILAVLSWLPGLAPFPVKRISVANTAILYHDGRA
LATCESGPPMRVLLPGLETIGWWSNATESGSTKGHAARGLARFMREWTTGHPKVDP
ISGEFIAFHNTVIRPYISYSVIPPSRLSAAGRSSTTAHSQGAENGLQPSSLISLAGAPIQPS
PSSPKMSHDFGVSLTHTVLVDPPLLLDPLNLLRGKPILTYDTSPEAKLRFGVFKRYSP
HDVHWVDTHPGVVLHVANTWDEYGDEVLADGDKNVVGINMLVPRMNSASLVYA
TGNIPSPHASLQQDEETDEEGEPEECTLHYFYFSLPSSGTLAPHNTESDTPPTSSTSHLA
SLPTSRASSTTLLHSFPISAIPLELPTVSPHTAMRCARYIYGCTMREGTFSGGKVQCLA
KFDVLTLIDRGKRESVSDGIERRPVDQRSVQEIIQQQLEAQQSESAIKIFNMPPGWCC
QEATFVPRTNQQSYSLPASSDEEEDDGWLLSYVFDESQLDGAGMPRADARSELWIID
ARNMKDVVARIVLPQRVPYGFHGSFFTEDQIAQQQPIDDIRHSPTQPCRHHFLHQFTP
PLIC
SEQ ID NO: 112 (Neohortaea acidophila) (XP_033588727.1)
MPLAGQKRKRAASGDILSTPQPKHPYLTGNFAPIHNTLPLTPCSHTGTIPPELSDGQY
VRNGSNPVTNEDLGRDAHWFDGDGMLSGVSFRKDDTGIVPEFVNQFILTDLYLSALS
SPRLKVPILPSIATLVHPLYSIFYVTLRIFRTVLLVLLSHLPGSKQKIRKISVANTNVLY
HDGRALALCESGPPMRVQLPELETVGWYNGDRAQGEPEKERGAALGDGSGLLQFM
REWTTAHPKVDPQTKEMLMFHSSFAPPYVQYSIIPKEKSPSDEAVASQKLLNAPVPG
VRKAKMMHDFGVSLTHTVIMDLPLTLDPVNQLKGLPPVTYDASQPSRFGVFPRRSPE
QVQWFETDAACIYHTANTWDDVNATGSPTAVNMLACRLTSATLIYAAANIAPPAPK
SNKRVAQANRRMPFFAKYNEEEDPSLWERAPLLESPSEEKDTFIRVDALESPSSNPAA
FDEEQCRLYYYHFDLNNGALTHQWALAAIPFEFPSVRPDLEMQHARYVYGCSTTTT
SFSSALGKATKIDALVKVDVNRLIANGKSHPPQSVTGVVDPRPMAAILSANDPHDPIQ
VFRMPPGWFAQEPRFVPAASNASEDDGYLLFYAFDEAQLNAAGDAPDDVSDSRAKS
ELWIIDAKTMKDVVARVQLPQRVPYGLHGTWFSAEQIRAQRPAEQLRSTREVLGAK
EVGMWMGVRDWCEKMLG
SEQ ID NO: 113 (Peltaster fructicola) (QIW99276.1)
MAFTSQKRKRAVSELMQPTPQPKHPYKSGNFAAIHQTLPLTPCTYTGTIPKELGDGE
YVRNGSNPLGSGSQEDIGRDYHWFDGDGMLAGVWFERDASGEISPQFVNQYILTDV
YLSAIGSRHLKKPILPSIATLVDPLASFFWVTIQIFRTLLLVLLSHLPGSKQKIKKISVAN
THIMYHDGRALALCESGPPMRIQLPGLETVGWFNGSVAKGEPLKTITEKEPVLGGDD
SLYSFMREWTTAHPKVDPKTKEMLAFHSCFAPPYIQYSILPESSTVEKQKMLNAPVP
GIRGARMMHDFGASSAHTIIMDLPLSLDPTEQFKGRPTVNFDATQSSRFGVFPRRRPE
DVRWFETDACCIFHTANSWDSLTMGKTTGVNMLACRLTSATLVFAAGNIAPPAERK
KVKADIIKKRRMPFFAKYDQSIDVTAWKDSEQLGTPEDEKQAFIRIVPEPIAGSGPGD
YDDEQCRLYYYHFDLETEAIAHQWALSAISFEFPSVNPQYEMAAARYVYGCSTTDES
FGQALGKATKIDVVVKMDVQTLIARGKKQPPASVTGVVDERLIEEVLEAGSDDPIQA
FKMPDGWYAQEPRFVSASNPTSEDDGFLIWFAFDESQLLDNGDVPADNTTQRAKSE
LWIVNAKDMRTVLARVQLPQRVPYGLHASWFEQSQIQQQRPVAEVRSTAIALSPNPP
GLWMEIRDWVEACLG
SEQ ID NO: 114 (Lizonia empirigonia) (KAF1365755.1)
MSRHGHSKKHTHKHPYLGGNFAPVTRTWTPTSVTWTGNLPKELNGGMYVRNGGNP
IANSDLGREAHWFDGDGMLSGVWFGRSTNDANELHVQFVNQFVLTDVYLSSLENN
TLRTPILPSIATLTNPASSLIWLIWRIMRTVLIVLLSHLPGSKFAIKRISVANTSVLYHDG
RALATCESGPPLRLTLPQLETVGWFDGNAVEGEPKTEDKKEEMTLGGTGPLSWMRE
WSTGHPKVDNATGEMVLFHCSFAPPYVQYSLIPPTGSNITDHQSADDAGRIINAAVP
GCTGGKLMHDFGVSKHSTVILDLPLSLTPLNLAKNSPVVAYEPHKPSRYGIFPRQHPE
AVRWFETEGCCIFHTANTWDEYDSDGNVAAVSLLACRLTSASVIFSAGNIAPPPHPK
HKDAISEKPMSFFAKYDSDADDQDPEKILSDETSPLLTYRDAVPNPFGASSSLPISNDD
EEQCRLYYYRFSLESMTENVITHQFALAAIPVEFPTVSPLTAMMSARYIYACSTSNES
FGAALGKATKIDVIVRFDVQALLARAQIEPPDAITGCVDNRTLGQIMASNDPNDPIKA
FRLPEKHYGQEATFIPRSSRGSATNKNTAFDEDDGYLVFYVFDEHQLDEAGECKENA
RSELWVLDAQTMNRVECKIQLPTRIPYGLHGNWFTEEQIARQKGVEKVRALPAVAP
VTKWSRIKRSIHGRLG
SEQ ID NO: 115 (Cercospora beticola) (XP_023459336.1)
MFQLTSEGGGSRFITNPLAGQKRKRSPSTNIQPTPQAKHPYLSGNFAPIQQTLPLTPCT
YTGSIPEELAEGEYIRNGSNPVSNDDLGRDSHWFDGDGMLSGVSFTKDEQTGEVTPE
FVNQFVLTDLYLSTLGSDRLRVPILPSIATLVSPVASFIWVTIRILRTVLLVLLSHLPGS
KQKIKKISVANTNVIYHDGRALATCESGPPMRIQLPGLETVGWYNGVSAEGEPVEER
EEQGKDKAFGQDGGLISFMREWTTAHPKVDPVSKEMLLYHASFAPPYVQYSVIPKA
SSTTGTKGKRLVNVGIPKVSGAKMMHDFGVSSQHTIIMDLPLTLDPINQLKGLPTVH
YDSSKPSRFGVFPRHSPAETQWFETDGCCIFHTANSWDTVESDGHVSSVNMLACRLT
SATLIYAAGNMAPPPLKPLTSSVAKKTKRMPFFSSYDTGNGHTNFDPDQPLDEKEPL
LRVGERLYYYSFDLNSTNITHQWALSAVPFEFPSVHPGREMSEAQYVYGCSTTTTDF
GAALGKATKIDAIVKIDAKKLIQRGRANPPKSVTGVVDIRTMAQVLETSDPEDSIRVF
VMPPGWFAQEPRFVPRQNPASEDDGYLLFYAFDEGQLTPDGDVPADNCEGRAKSEL
WIVDAKNMQKVVGRVQLPQRVPYGLHGSWFSAEMIKNQRAVESIRSTAKALERDG
AGGVWMKVRDTLEGWLG
SEQ ID NO: 116 (Fusarium sporotrichioides) (RGP69130.1)
MDSLQHIVPAVVSISHPKEQSPPRTLRHPYLIGNFAPIHKTLNLTPCTHSGCIPPELTDG
QYVRNGGNPVSHEDLGRDAHWFDGDGMLSGVLFRKGHSDGQIVPEFVNQYILTDL
YLSRKTTSVSSPIMPSITTLVNPLSSLLKILLATFRTMFLVFLSNLPGSQQALKRTSVAN
TAILYHDGRALATCESGPPMRIQLPSLDTVGWYNGAHAEGEPEQALSENKIEPFGGD
SLFKSMREWTTGHPKVDPVSGEMILYHNTFIQPYVHYSVLPKTDVQAPTTGRLVNQ
AVPGVSGARMMHDFGASRAHTIIMDLPLTLDPMNLAKNKEVVSYDPSKPSRFGVFP
RHEPSNVRWFQTAPSCIFHTANSWDTKSVNGTSSVNLLACRMTSSTVVYTAGNIKPP
VKPKRHNNRLPHAKENISQWDEKDIQRFEAAPMLESPTEKLYGDDYFSSSDEADDYS
QCRLYYYEFDMSATSTNNVINQWALSTIPFEFPSVRPDREMQDARYIYGCSTSTSCFG
VALGRADKVDLLVKMDAKTLVERGKRMNTRPVTGCVDRRSAREILDSQDENDPIKI
FRLPPRHFAQEPRFVPRAGVAEEDAGYLLFYVFDESQILPNGDCPSSSASELWILDAQ
NMRDVVAKVRLPQRVPYGLHGTWFSARDIEEQRAIETLRSLEAVQRKKEIWVNDGG
SIARSWMAFREKLERAVG
SEQ ID NO: 117 (Ramularia collo-cygni) (XP_023621334.1)
MQEMAHAGQKRKRTPTPHIPPTPQAKHPYLAGNFAPISQTLPLTKCTWTGHIPQELA
DGQYVRNGSNPVSNEDLGRDAHWFDGDGMLSGVLFRRDTTSGDITPEFVNAYVLT
DVHLSATSSPRLIRPILPSIATLVDPLASFIYVTWRIIRTILLVILSHLPGSKQAIKKISVA
NTNIVFHDGRALATCESGPPIRIQLPGLETVGWYNGSTAQNEPPQPLPSAEECRFLGQ
DSGLIGFMREWTTAHPKVDPVTQEMLMFHSSFAPPYVQYSIVPAQQTSSTSHLPRRK
MLNAAVPGVTSAKMMHDFGCSSTHTVIMDLPLSLDPLNTFKGTPTVAYDPSKPSRF
GVFPRHSPQQVTWYETDASIVMHTANTWDEVDETTRQVTGVNMLCCRLTSATLVF
AAGNIAAPAPIPRKPAVVAKRKRMSFFSPYNAAVEPTHVERHVDEKEPLLHTVENTN
LDDEEELMEEDQCRLYYFHFSLTNPSTIISQYALTAIPFEFPSTHPAKSMQSARYIYGC
STTTSSAFGAALGKATKIDAVVKVDAGTLIERGRRNPPKSVSGVVDSRPVEDISVGDE
VIGMFKLPPGWFAQEPRFVPRDGGQGEDDGWLLFYAFDESQLDATTGEVEREEGVH
SELWILDARDMDSVVARVVLPQRVPYGLHGSWFPKEEIEGQRGVVEFRREIQGTEEG
RGVWAAVRRGLERGLA
SEQ ID NO: 118 (Zymoseptoria tritici ST99CH_3D1) (SMR64689.1)
MVHAGQKRKRSPSENIPPTPQPRHPYLTGNFAPIQQTLPLTPCTYTGQIPEELADGEY
VRNGGNPVSNDDLGRDAHWFDGDGMLSGVAFTKDEATGRIQPEFVNQFILTDVYLN
TLASPKLRVPILPSIATLVNPLASFIWVTIRIFRTILLVILSHLPGSKQAIKKISVANTNVV
YHDGRALATCESGPPIRIQLPGLETVGWYDGANAQGEPMKEDTAKEATLGQNSGLIS
FMREWTTAHPKVDPTTKEMLMFHSSFAPPYVQYSIVPKSSESEGGEGRTRRSKILNA
AVPGVTSAKMMHDFGVSPTHTVIMDLPLSLDPINQLKGLPTVTYTPSKPSRFGVFPRR
QPESVQWFETDASCIFHTANTWDDVSSAGETTAVNMLACRLTSATLVFAAGNIAPPI
EKLKAPPVKAKRRMPFFSKYDSATEVTIHDLDSPTDEKEPLLTVTTVDEAGEVEEPFE
EDQCRLYYYHFDMTTHNITHQWALSSIPFEFPSVRPDVEMQNAQYIYGCSTTTTSFGS
ALGKATKIDALVKIDAKTLIERGRRTPPPSVTGVVDPRNMTQVLADQHDPTDPVRVF
TMPEGWFAQEPRFVPSSSNASEDDGYLLFYAFDEGQLSSLGDVPDDVSPSRAKSELW
IVNAKNMTDIVARVQLPQRVPYGLHGSWFTAEQIAEQKPVECIRTTAKALEVREKGV
WMSVRSWVEGVLG
SEQ ID NO: 119 (Zymoseptoria tritici ST99CH_1A5) (SMY30021.1)
MVHAGQKRKRSPSENIPPTPQPRHPYLSGNFAPIQQTLPLTPCTYTGQLPEELADGEY
VRNGGNPVSNDDLGRDAHWFDGDGMLSGVAFTKDEATGRIQPEFVNQFILTDVYLN
TLASPKLRVPILPSIATLVNPLASFIWVTIRIFRTILLVILSHLPGSKQAIKKISVANTNVV
YHDGRALATCESGPPIRIQLPGLETVGWYDGANAQGEPKKENTAKVVTLGQNSGLIS
FMREWTTAHPKVDPTTKEMLMFHSSFAPPYVQYSIVPKSSESEGGEGRTRRSKILNA
AVPGVTSAKMMHDFGVSPTHTVIMDLPLSLDPINQLKGLPTVTYTPSKPSRFGVFPRR
QPESVQWFETDASCIFHTANTWDDVSSAGETTAVNMLACRLTSATLVFAAGNIAPPI
EKLKAPPVKAKRRMPFFSKYDSATEVTIHDLDSPTDEKEPLLTVTTVDEAGEVEEPFE
EDQCRLYYYHFDMTTHNITHQWALSSIPFEFPSVRPDVEMQNAQYIYGCSTTTTSFGS
ALGKATKIDALVKIDAKTLIERGRRTPPPSVTGVVDSRNMTQVLADQHDPTDPIRVFT
MPEGWFAQEPRFVPSSSNASEDDGYLLFYAFDEGQLSSLGDVPDDVSPSRAKSELWI
VNAKNMTDIVARVQLPQRVPYGLHGSWFTAEQIAEQKPVECIRTTAKALEVREKGV
WMSVRSWVEGVLG
SEQ ID NO: 120 (Zymoseptoria tritici (Speckled leaf blotch fungus)
(Septoria tritici) (UPI001309FD1A)
MVHAGQKRKRSPSENIPPTPQPRHPYLTGNFAPIQQTLALTPCTYTGQIPEELADGEY
VRNGGNPVSNDDLGRDAHWFDGDGMLSGVAFTKDEATGRIQPEFVNQFILTDVYLN
TLASPKLRVPILPSIATLVNPLASFIWVTIRIFRTILLVILSHLPGSKQAIKKISVANTNVV
YHDGRALATCESGPPMRIQLPGLETVGWYDGANAQGEPKKEDIAKEATLGQDSGLIS
FMREWTTAHPKVDPTTKEMLMFHSSFAPPYVQYSIVPKSSESEGGEGQTRRSKILNA
AVPGVTSAKMMHDFGVSPTHTVIMDLPLSLDPINQLKGLPTVTYTPSKPSRFGVFPRR
QPESVQWFETDASCIFHTANTWDDVSSAGETTAVNMLACRLTSATLVFAAGNIAPPI
EKLKAPPVKAKRRMPFFSKYDSATEVTIHDLDSPTDEKEPLLTVTTVDEAGEVEELFE
EDQCRLYYYHFDMTTHNITHQWALSSIPFEFPSVRPDVEMQNAQYIYGCSTTTTSFGS
ALGKATKIDALVKIDAKTLIERGRRTPPPSVTGVVDPRNMTQVLADQHDPTDPVRVF
TMPEGWFAQEPRFVPSSSNASEDDGYLLFYTFDEGQLSSLGDVPDDVSPSRAKSELW
IVNAKNMKDVVARVQLPQRVPYGLHGSWFTAEQIAEQKSVECIRTTAKALEAREKG
VWMSVRSWVEGVLG
SEQ ID NO: 121 (Zymoseptoria tritici (Speckled leaf blotch fungus)
(Septoria tritici)) (UPI00130ABF23)
MVHAGQKRKRSPSENIPPTPQPRHPYLTGNFAPIQQTLPLTPCTYTGQIPEELADGEY
VRNGGNPVSNDDLGRDAHWFDGDGMLSGVAFTKDEATGRIHPEFVNQFILTDVYLN
TLASPKLRVPILPSIATLVNPLASFIWVTIRIFRTILLVILSHLPGSKQAIKKISVANTNVV
YHDGRALATCESGPPMRIQLPGLETVGWYDGANAQGEPKKENTAKVVTLGQDSGLI
SFMREWTTAHPKVDPTTKEMLMFHSSFAPPYVQYSIVPKSSECEGGEGQTRRSKILN
AAVPGVTSAKMMHDFGVSPTHTVIMDLPLSLDPINQLKGLPTVTYTPSKPSRFGVFP
RRQPESVQWFETDASCIFHTANTWDDVSSAGETTAVNMLACRLTSATLVFAAGNIAP
PIEKLKAPPVKAKRRMPFFSKYDSATEVTIHDLDSPTDEKEPLLTVTTVDEAGEVEEP
FEEDQCRLYYYHFDMTTHNITHQWALSSIPFEFPSVRPDVEMQNAQYIYGCSTTTTSF
GSALGKATKIDALVKIDAKTLIERGRRTPPPSVTGVVDPRNMTQVLADQHDPTDPVR
VFTMPEGWFAQEPRFVPSSSNASEDDGYLLFYAFDEGQLSSLGDVPDDVSPSRAKSE
LWIVNAKNMKDVVARVQLPQRVPYGLHGSWFTAEQIAEQKSVECIRTTAKALEARE
KGVWMSVRSWVEGVLG
SEQ ID NO: 122 (Zymoseptoria tritici (Speckled leaf blotch fungus)
(Septoria tritici)) (UPI00130CFB6E)
MVHAGQKRKRSPSENIPPTPQPRHPYLTGNFAPIQQTLPLTPCTYTGQIPEELADGEY
VRNGGNPVSNDDLGRDAHWFDGDGMLSGVAFTKDEATGRIHPEFVNQFILTDVYLN
TLASPKLRVPILPSIATLVNPLASFIWVTIRIFRTILLVILSHLPGSKQAIKKISVANTNVV
YHDGRALATCESGPPMRIQLPGLETVGWYDGANAQGEPKKENTAKVVTLGQDSGLI
SFMREWTTAHPKVDPTTKEMLMFHSSFAPPYVQYSIVPKSSECEGGEGQTRRSKILN
AAVPGVTSAKMMHDFGVSPTHTVIMDLPLSLDPINQLKGLPTVTYTPSKPSRFGVFP
RRQPESVQWFETDASCIFHTANTWDDVSSAGETTAVNMLACRLTSATLVFAAGNIAP
PIEKLKAPPVKAKRRMPFFSKYDSATEVTIHDLDSPTDEKEPLLTVTTVNEAGEVEEL
FEEDQCRLYYYHFDMTTHNITHQWALSSIPFEFPSVRPDVEMQNAQYIYGCSTTTTSF
GSALGKATKIDALVKIDAKTLIERGRRTPPPSVTGVVDSRNMTQVLADQHDPTDPIR
VFTMPEGWFAQEPRFVPSSSNASEDDGYLLFYAFDEGQLSSLGDVPDDVSPSRAKSE
LWIVNAKNMTDIVARVQLPQRVPYGLHGSWFTAEQIAEQKPVECIRTTAKALEVRE
KGVWMSVRSWVEGVLG
SEQ ID NO: 123 (Zymoseptoria tritici (Speckled leaf blotch fungus)
(Septoria tritici)) (UPI0013063113)
MVHAGQKRKRSPSENIPPTPQPRHPYLSGNFAPIQQTLPLTPCTYTGQLPEELADGEY
VRNGGNPVSNDDLGRDAHWFDGDGMLSGVAFTKDEATGRIQPEFVNQFILTDVYLN
TLASPKLRVPILPSIATLVNPLASFIWVTIRIFRTILLVILSHLPGSKQAIKKISVANTNVV
YHDGRALATCESGPPIRIQLPGLETVGWYDGANAQGEPMKEDTAKEATLGQNSGLIS
FMREWTTAHPKVDPTTKEMLMFHSSFAPPYVQYSIVPKSSECEGGEGQTRRSKILNA
AVPGVTSAKMMHDFGVSPTHTVIMDLPLSLDPINQLKGLPTVTYTPSKPSRFGVFPRR
QPESVQWFETDASCIFHTANTWDDVSSAGETTAVNMLACRLTSATLVFAAGNIAPPI
EKLKAPPVKAKRRMPFFSKYDSATEVTIHDLDSPTDEKEPLLTVTTVNEAGEVEELFE
EDQCRLYYYHFDMTTHNITHQWALSSIPFEFPSVRPDVEMQNAQYIYGCSTTTTSFGS
ALGKATKIDALVKIDAKTLIERGRRTPPPSVTGVVDSRNMTQVLADQHDPTDPIRVFT
MPEGWFAQEPRFVPSSSNASEDDGYLLFYAFDEGQLSSLGDVPDDVSPSRAKSELWI
VNAKNMTDIVARVQLPQRVPYGLHGSWFTAEQIAEQKPVECIRTTAKALEVREKGV
WMSVRSWVEGVLG
SEQ ID NO: 124 (Zymoseptoria brevis) (KJY02167.1)
MVHAGQKRKRSPSENIPPTPQPRHPYLTGNFAPIQQTLPLTPCTYTGQIPEELADGEY
VRNGGNPVSNDDLGRDAHWFDGDGMLSGVAFTKDEATGRIQPEFVNQFILTDVYLN
TLASPKLRVPILPSIATLVNPLASFIWVTIRIFRTILLVILSHLPGSKQAIKKISVANTNVV
YHDGRVLATCESGPPMRIQLPGLETVGWYDGANAQGEPVNEDTAKEEATLGQDSGL
ISFMREWTTAHPKVDPTTKEMLMFHSSFAPPYVQYSIVPKSSESGGVEGQTRRNKILN
VAVPGVTSAKMMHDFGVSPTHTVIMDLPLSLDPINQLKGLPTVTYTPSKPSRFGVFP
RRQPESVQWFETDASCIFHTANTWDDVSSAGETTAVNMLACRLTSATLVFAAGNIAP
PMEKLKAPPVKAKRRMPFFSKYDSATEVTIHDLDSPTDEKEPLLTFTTLDEAGEVEEL
FEEDQCRLYYYHFDMTTHNITHQWALSSIPFEFPSVRPDVEMQNAQYIYGCSTTTTSF
GSALGKATKIDALVKIDARTLIERGRRTPPPSVTGVVDPRNMTQVLAGQHNPTDPIRV
FTMPEGWFAQEPRFVPSSSNASEDDGYLLFYAFDEGQLSSLGDVPDDVSPSRAKSEL
WIVNAKNMTDIVARVQLPQRVPYGLHGSWFTAEQIAQQKPVECIRTTAKALEVREK
GIWMSARSWVEGVLG
SEQ ID NO: 125 (Fusarium solani) (KAH7254831.1)
MSVVVDVSTSGFVRHCETLYWSSTAKLAPFSITGGLGIEWVWSCYLSGNFAPVQQTT
PLTPCVYTGNIPPELQGGQYVRNGGNPVGHEDLGRDAHWFDGDGMLSGVAFRGISP
EDGRIIPEFANQFILTDLYLSKKTTAVSSPIMPSITTLVNPLSTLFQIILSVLRTVFLVLLS
HLPGSQQAIKRISVANTSILYYDGRALASCKSGPPIRIQLPSLDTVGWENGIQAEGEQE
DFKSQLDARQFGGHDALSFLKEWTTGHPKVDKRTGEMLLYHNTFLSPFVHYSVIQSS
LSKNSNTPKLKLINEPVAGVSGARMLHDFGASHTQTIIMDLPLSLDSPNVLRNREVVS
YDPSKPSRFGVFPRHKPHEDTTAEGKVVSVNLLACRMASATLIYSAGNIAPPNVSSNT
KHIELEKTPLTRKTRYRSPRKTMCHYEKGSVLESSASTPNAPNYPSTHASEDNDQCR
LCYYEFDLSATEKNQVSHEWALSAIPFEFPSVRPDCEIQRARYVYGCTTSSSCIGVAL
GKAVKIDVIAKIDSKTLIQNGKDMNITPVTGCVDERTVGEILDEDDKEGSIQCFRLPR
NHFAHEPRFIPRSSSDEEDDGYLLFYVFDKSQLTMSGECPQSAVSELWILNAESMRN
VVAKITLPQRVPYGLHGIWFSHSDIEKQRDVKTFRSLEQLRTRKDEGVFGRKQWWC
SWIRWRDVLEKAVG
SEQ ID NO: 126 (Polychaeton citri CBS 116435) (KAF2724708.1)
MAVVGQKRKRAVSGNIQPTPQAKHPYLSGNFAPIHQTLPLTPCMYSGTIPEELADGE
YVRNGSNPVSNEDLGRDAHWFDGDGMLAGVSFRKDKNGRIHPEFVNQFVLTDLYL
NTISSPRLRVPILPSIATLVNPVASFFLVTLRILRTLILVLLSHLPGSKQSIKKISVANTNI
LFHDGRALATCESGPPIRVQLPSLETVGWYDGSCAEGEPERLSPEKEATFGQDGGLV
SFMREWTTAHPKVDPETKEMLMFHSCFAPPYVQYSIVPQTSDGVSEQALTLRKEKV
LNSPVPGITSAKMMHDFGASLTHTVIMDLPLSMDPINQFKGKAPVTYDSEKPSRFGV
FPRRSPQDVKWFETDASCIFHTANTWNNTNMKGEVTAVNMLACRLTSATVVFAAG
NIAPPVPPKKTIASAKKRMPFFSKYNVKEEPAIFDRAPLLESPSEEKEALIQLSDDHSP
WAMDDLFDEEQCRLYFYQFDLRSGQIKHQWALSAVPFEFPSVRPNLEMQEARYIYG
CSTTTTEFGSALGKATKIDALVKVDAASLIQSGQARPPKSVTGVLDSRSVLQILDHDD
VNDPIRIFHMPEGWYAQEPRFVPSSTNKSEDDGYLLFYAFDECQLGDNGEVPLDSSPI
RAKSELWIIDAMDMKTIVGRVHLPQRVPYGLHGIWFTSEQIAEQRPVDSIRSATEALS
KKDEGAWMVVRDWVEKLLG
SEQ ID NO: 127 (Aureobasidium subglaciale EXF-2481) (XP_013345212.1)
MPLFAQKTGLPQHKHAYLSGNFAPISQTMPLTKCTYEGVLPSELAGGEYVRNGGNP
VSNEDLGRDAHWFDGDGMLSGVSFTKQPDGSVQPEFVNQYILTDIFLSTVASPRLRS
PILPSIATLVNPLASLITIVLRILRTIVLVILSFLPGSTRAIKKISVANTSILYHDGRALATC
ESGPPMRIALPGLETVGWYDGNRAEGEPDAHDKHDPSFSGNGLLGFMREWTTAHPK
VDPTTKEMMLFHSTFFPPYVQYSIIPPTSARSETNKGIVQRPVPGITGAKMMHDFGVS
LQHTIIMDLPLSLDPLNLARNKPVIDYDATKPARFGVFPRHHPDQVKYFETTACCIFH
TANAWDDFDASGQVETVHLLTCRLTSACLVFSAGNIAAPRPTKETVKAVKKNMPFF
SKYNASEQCLYDGAPTLESPLLAREPLIQITSDDEAIPVPSDETDGQVEEIPDEDDQCR
LYHYAFSLKSSRITAQYSLSAISFEFPSVHPEFDMQEARYVYGCSTTVSSFNAALGRS
VKIDALAKIDAKALIEKGDRLMQLGKLEPVTGCVDTRTVQEVIDSALDNDPIQVFRM
PAGWFAQEPRFVPRKAAPGQTLAEDDGYLLTYAFDESQLCADGEVPCDADTANRA
KSELWIIDAKDMRSVVGRVALPQRVPYGLHGVWFPEEDILGQKAVDTVRSISEAKSN
KNEGIWMNIRASIEGMLS
SEQ ID NO: 128 (Ceraceosorus bombacis) (CEH13466.1)
MTSGLLNQGERRDPHPYLSGNYAPVRTELPLTACRYEGRVPDEFRDGQYCRNGGNP
LSGEDEMRDAHWFDGDGMLTGVLFRRMPDGSVRPEFINRYVLTDVLLATSPRSTRP
LLPSIATFSSPYIPLLSVLLGVLRTLALCLLTWLPGSGCSAVEEQERRLKRISVANTSV
WFHDGKAFAGCESGPPLRVLLPGMETAGWWSGQDVADRDVDDSEVETKHRKSHG
WGKSGVAGLFAEMTTAHPHVDPKTGELLLFHMSFFAPFLRVSIIPPKKGSAEGDSRS
RDQRQGAQAPLLGVEVPGIAQPKQMHDAGWSSRHGIMLDLPLSLDPRNMLRGRPM
LHYDSAGPTRFGIFPRREPEKVVWFEEAQACLIFHTANAWEEQSEDGRESGAVSLLA
CRLNSATLVYSAGNTIPPPHALPPGGRAEQCRLHYWRFPDIEQDRQPALTLPRRPSHS
FALSAVPFEFPTMNEACAMSEASFVYGASMRSGSFDAGLGRQSAKIDCLAKIDAAKL
VRRGLEQGIGTEVAGFCVDDRTVQDILDEQGAQFECAHGSSSDQSSASAAIRIFALPP
NHFAQEATFVPRRGAMQEDDGWLVFFVFDESQLDDDGRVLHDATSELWILEAKTM
RDVICRVKLPQRVPYGLHGHFFNAQEIASQEPLNDEQIRTWILASQGVVGPGMGTGG
KPAVDLYAGPLAFQRRVTAAMRSTLESWLL
SEQ ID NO: 129 (Zasmidium cellare ATCC 36951) (XP_033671248.1)
MPLAGQKRKRTSSENILPTPQAKHPYLSGNFAPIQQTLPLTKCTFMGRIPEELADGEY
VRNGSNPVSNEDLGRDAHWFDGDGMLSGVSFTKDKATGEVQPDFVNQFVLTDLYL
NTVSSPRLRVPILPSIATLVNPVASFIYVTLRILRTILLVILSHLPGSKHPIKKISVANTHV
LYHDGRALATCESGPPIRIQLPGLETVGWYDGASAEGEPAKETEDKESVLGQDGGLI
SFMREWTTAHPKVDPNTKEMIMFHSSFAPPFVQYSIIPQTAQVESGVQNGDASGKIL
NAAVPGVKSAKMMHDFGVSREHTIIMDLPLSLDPINSLKGLPPVSYDSSRPSRFGVFP
RRKPKQVKWFETDASCIFHTANSWDEADSSGQTATVNMLACRLTSATLIFAAGNIAP
PVEKREKAVAIAKKRMPFFSKYDQDAEQTYFEQSTALESPCEEKEPLLHIAKADRED
DYDDLFNEDQCRLYYYQFDLRSGSINYQWALSSIPFEFPSVRPDLEMSAARYIYGCST
TTTNFGSALGKATKIDALVKIDATALITRGRTTPPRSVTGVVDSRSMAQVLESNDPHD
PIQVFQMPEGWYAQEPRFVPASSNASEDDGYLLFYAFDESQLDSNGDVPTDSSPLRA
KSELWIVDAQGMKDIVARVKLPQRVPYGLHGTWFSAEQIKEQRPVDCIRTTAKAME
GKGEGWWMSTRDQLERMLG
SEQ ID NO: 130 (Rachicladosporium sp. CCFEE 5018) (OQO21131.1)
MVVAGQKRKRGGSDNILPTPQPRHPYLTGNFAPIDKTIPLTPCTYTGTIPEELADGEY
VRNGSNPVSNSDLGRDAHWFDGDGMLAGVLFRKDETTGSIQPEFVNQYILTDVYLS
SIGSKRLKVPILPSIATLVNPLSSFFWVILRILRTILLVILSHLPGSKQKIKKISVANTNIV
YHDGRALALCESGPPLRIQLPGLETVGWYDGATAEGEPVDAQSTEKERVLGEGSGLI
SFMREWTTAHPKLDPKSKEMLMFHASFAPPYVQYSIVPQSKTTDQAGAPMQKVLNA
AVPGVRGARMMHDFGVSSSHTIIMDLPLSLDPLLQLQGKPPVSYDSSKPSRFGVFPRR
EPEKATWFETDACCIFHTANSWDVVDASGNTTAVNMLACRLTSATMIFATGNIAPPA
PPKKTSTDALPKKRMSFFSKYNLDTEASVYERHGRVESTEEEKEALLHVTSTEISSIEN
DDDGEDHHFDEDQCRLYYYHFDLSTSQIIHQWALASIPFEFPSVHPDLEMSEAKYVY
GCSTSTTSFGSALGKATKIDILVKVDVTTLIARGRANPPRSVSGVVDPRPMADVITSN
DPADPIRVFQMPDGWFAQEPRFVPAATKRSEDDGFLLFYAFDEAQLTLTGDAPLDEG
EARARSDLWVVDARDMRTVMAQVRLPQRVPYGLHGGWFTGEQVRGQRSVERVRS
VEKVLEEKQGGVWMGIRDSIEKFLA
SEQ ID NO: 131 (Cercospora berteroae) (PPJ52044.1)
MFQLTRAGGGSRFITNTRAGQKRKRSPSTNIQPTPQAKHPYLSGNFAPIQQTLPLTPC
TYTGSIPEELAEGEYIRNGSNPVSNDDLGRDSHWFDGDGMLSGVSFTKDEHTGDVTP
EFVNQFVLTDLYLNTRGSDKLRVPILPSIATLVSPVASFIWVTFRILRTVLLVLLSHLPG
SKQKIKKISVANTNVIYHDGRALATCESGPPMRIQLPGLETVGWYNGVSAEGEPIDEK
DEQDKDKAFGQDGGLISFMREWTTAHPKVDPVSKEMLLYHASFAPPYVQYSVIPQA
SGTSAAAGTRLVNVGIPKVSGAKMMHDFGVSSQHTIIMDLPLTLDPINQLKGLPTVH
YDASKSSRFGVFPRHKPEETQWFETDGCCIFHTANSWDNVEPHGHTSSVNMLACRL
TSATLIYAAGNMAPPPLKPLSSSVVKKKKRMPFFSSYDTGNGQTNFDPDQPLDEKEP
LLRVGERYTSIYEDERDPLAEDQCRLYYYSFDLNSTNITHQWALSVVPFEFPSVHPGR
EMSEARYVYGCSTTTTDFGAALGKATKIDAIVKMDAKKLIQRGRANPPKSVTGVVDI
RTMAQVLETSDPEDPIQVFVMPPGWFAQEPRFVPCKDSTSEDDGYLLFYAFDEGQLT
PDGDVPADTCEGRAKSELWIVDAKNMQKVVGRVQLPQRVPYGLHGSWFSAEMIKN
QREVASTRSTATALDRNERGGLWMKVRGTLEGWLG
SEQ ID NO: 132 (Baudoinia panamericana UAMH 10762) (XP_007674358.1)
MLEGAPTLAAGQKRKRTSSSHTIQPTPQAKHPYLTGNFAPIQQTLPLTPCTYTGDIPD
ELADGEYVRNGGNPVSNEDLGRDAHWFDGDGMLSGVSFRRDQKAGRIVPEFVNQY
VLTDLYLSTLSSPRLRVPILPSIATLVNPVASVIYVTFRIFRTLFLVLLSFLPGSKQKIKK
ISVANTHIVYHDGRALATCESGPPMRVQLPGLQTVGWYNGAWAEGERKAEEAQAK
KQQEVERVLGQDGGMIAFMREWTTAHPKLDPVTKEMIMFHSSFARPYVQYSIIPQEA
REPVDEKRSLQPQSKMLNAAVPGIYSAKMMHDFGVSTGHTVIMDLPLALDPMNQM
KGKPPMSYDSSQPARFGVFPRRHPEQVRWFETDACCIFHTANSWDTVDAAGYTTSV
NMLACRLTSATLVFATGNVAPPAARKPKTVAMAKRMSFFAKYDDPMNASVYERSA
LLESPLEEQEVPNRRGEAAADSEEYDSVWDENECRLYYYSFCLKTGQIEHQWALSTI
AFEFPSVRTDLEMQAARYVYGCGSSVTSFGSALGKATKIDLLIKVDVQTLIARGLQR
PPRSVTGSVDTRSIEEILASNDDNDPIRAFQLPEGWFAQEPRFVPASTNSSEDDGWLV
FYAFDESQLTVDGEVPSDNSPLRARSELWIVNARDVRTLVARVHLPQRVPYGLHGS
WFTADQIREQRAIENVRSTAKALEKREVGWWMSCRDVIERMLG
SEQ ID NO: 133 (Acidomyces sp. 'richmondensis') (KXL45605.1)
MLSGLPAPLAGSKRKRASSFGEINPTPQARHPYLSGNFAPIHQTVHLTPCSYTGRIPRE
LAGGEYVRNGSNPVSNEDLGRDAHWFDGDGMLSGVSFAKDATTGEIRPHFANQFV
LTDLYLSAISSPRLRVPILPSIATLVNPLVSFIYVTLRILRTVALVILSFLPGSAQSISRISV
ANTHVVYHDGRALALCESGPPMRVQLPNLETVGWYNGSWAEGEREKVRQTEKPVV
KEAVLGQGSGIISFMREWTTAHPKIDPVTKEMMLFHASFMPPYVQYSILPQQSGPLSE
SAASFAQKEKMLNEAVPGVTGGRMMHDFGVSLTHTVIMDLPLSLDPMNQLKGLPPL
VYHSGKPSRFGIFPRRSPQSVRWFETDACCIFHTANTWDNLDSVGRTTDVNMIACRL
TSATVVFAAGNIAPPVPPKKTVAVSEQKKRRMPFFSKYDHDLELTIFERSTFLESPVE
EKEAFIRMDHSATHDNEEQSCSHLWDEEQCRLYYYKFNMTTNQITYQWALAAVPC
DFPSVNPQKEMQNARYIYGCSTSSKSFGAALGKATKIDVLVKVDAHRLIERGMRRPP
KSVTGCVDTRTLEQVLTSTDADDPIRAFKMPEGWFAQEPRFVPASSGSQEDDGFLLF
YAFDESQLNEAGEVPLDTSPLRAKSELWIIDAKGMKDIVCRVHLPQRVPYGLHGTWF
SEEQIQSQRPVHSVRTTSKARQGKEQNAWMRIRDCIETLLG
SEQ ID NO: 134 (Teratosphaeria nubilosa) (KAF2773889.1)
MLEATPFVVATQASTPQASTKRNRTSSSHELRPTPQPKHPYLSGNFAPIHQTLPLTPCS
YSGRIPEELAGGEYVRNGSSPVSNEDLGRDAHWFDGDGMLAGVLFRRDTEDGDVKP
EFVNQYVLTDLYLSTISSPHLKVPILPSIATLVNPLASVIYVTLRIIRTLMLVLLSFLPGS
KQSIKKISVANTHILYHDGRALATCESGPPIRIQLPELETVGWYNGAWAEGEKSDEAA
EEGQPDQKEKVLGEDGGLISFMREWTTAHPKVDPITKEMLMFHASFVPPYVQYSVIP
RQEKGSEKMTAGPSRQKMLNAAVPGVRGGRMMHDFGVSLKNTIIMDLPLSLDPIMQ
LRGLPPVTYDSGKPSRFGVFPRHRPDLVHWFETDACCIFHTANSWDTVDAAGRTTN
ANMLACRLTSATVIFAAGNIAPPVERQKVIAVAEQKKRRMSFFSKYDHESEKSIFERS
GLVESPSEEKEAFIKLSIDAAQAIDDKGPWDEEQCRLYYYDFDLTTGQIAHQWALAA
VPLEFPSVAPELEMQNARYVYGCSTSTTNFGSALGKATKIDVLLKVDAHTLIQCGKR
QPPRSVTGTVDTRNMDQVLASHDLEDPVRAFRMPEDWFAQEPRFVPAASRRSEDDG
YLLFYAFDETQLDGYGDIPSDSSELRANSELWVVDARTMKDVVCRVHLPQRVPYGL
HGSWFPEEQISGQRALEHVRTTAKATEGRENGLWMAIREWSERLLA
SEQ ID NO: 135 (Pseudocercospora fuligena) (KAF7196919.1)
MVFQYDSMEGGGGGGGIGSTVMSTLGKRKRSPSTNIERTPQPKHPYLSGNFAPIQQT
LPLTPCTYTGRIPEELADGEYVRNGSNPVSNSDLGRDAHWFDGDGMLSGVSFSKDEE
TGEIKPEFVNQFILTDLYLNAKSNGRLGVPILPSIATLVNPLASFIFVTLRIIRTILLVILS
HLPGSRQKIKKISVANTNVVYHDGRAMALCESGPPMRIQLPGLETVGWENGAVAEG
EPAKESEEKVAVLGENSGLISFMREWTTAHPKVDPQTKEMLMFHSSFAPPYVQYSIIP
QTARAEEEGAAPPTRKMLNAGVPGIKSAKMMHDFGVSSEHTVIMDLPLSLDPLNQL
KGLPPVAYNPDRPSRFGVFPRRDPKQTKWFETNACCIFHTANTWDERALDGSTTSVS
MLACRLTSATLIYAAGNMAPPVPPQAKALEESEAKRRKMPFFSKYDPEVDASFVESY
DEKEPLLRVPSLEEHLLKDPFAEEQCRLYYYNFDLASGIISHQWALSAVPFEFPSVRP
DLEMSQARYVYGCSTTTTDFGSALGKATKIDALVKIDATKLIQRGRTLPPRSITGVVD
GRTMSQVLISRDPRDPIKVFRMPEGWFAQEPRFVPRERSAHERQLNDDPLDEDDGYL
LFYAFDETQLLASGEVPDDTSKAKRAKSELWIVDARNMRDVIARVHLPQRVPYGLH
GSWFSAEQIAEQRPVERMRSMDEVMKERHDSGLWMKVRERIEMWLG
SEQ ID NO: 136 (Quercus suber (Cork oak)) (UPI000CE26881)
MSVYAGQKRKRSRVENIPFTPQARHPYLSGNFAPIQQTLPLTKCKFTGSIPHELGDGQ
YVRNGSNPVANGDLGRDAHWFDGDGMLAGVLFRRNSETGTIEPEFVNQYILTDLYL
STLASPALKVPILPSIAVLVNPIASIFYVMLRIVRTLLLVLLSHLPGSRQKIKRISVANTN
VVYHDGRALATCESGPPMRIQLPGLETVGWYDGANAEGETLRGAVDIKQEKVLGQ
DGGLISFMREWTTAHPKVDPKTKEMLMFHSSFAPPYVQYSIIPQTPRESLKQTTNTTQ
RKLLNAAVPGITSAKMMHDFGVSLQHTVIMDLPLSLDPMNQLKGKPPVTYDSSKPS
RFGVFPRHTPSEVTWFETDASCIFHTANSWDEDGEDGKTKNVNMLACRLTSASLVF
AAGNIAPPVEKVRRPPVGRSWKKQRMPFFSKYSAQDDVVDGSMLDLECPIEEKESLL
HITEPSAIVDCSEAELLEDQCQLYYYSFSLATKQIQHQWALSTIPFEFPSVHPDLEMQN
ARYIYGCSTTSTTFGAALGKATKIDALVKVDATTLIARGHARSPRSVTGAVDERSMA
QVLASQHDPTDPIKVFQMPPGWYAQEPRFVPATPSPSPALSREDDALSREDDGYLVF
YAFDEAQLDPATGDVPDDTHATLRARSELWIVDARDMSTLLARIHLPQRVPYGLHG
TWFAKEQIDAQRAVHELRTTRDVCLPLDERGRAGGSMMMMLRRWVEEMLG
SEQ ID NO: 137 (Elsinoe fawcettii) (KAF4553202.1)
MTVAGQKRKRGQASAKHPYLAGNFAPIQQVVPLTPCTYEGRIPDELAGGEYVRNGG
NPVSNEDLGRDAHWFDGDGMLSGVAFTKEEDGRIQPEFVNQYVLTDVYLSTISSPH
NKTPILPSIATLVNPVASFFWITWRVMRTIFLVVLSFLSGSKQAIKKISVANTSVWYHD
GRALAGCESGPPMRINLPGLDTVGWYDGCGAENEPKDEHAQDGKRIGGEGLLGWM
REWTTAHPKVDPVTKEMLLFHSSFAPPYVQYSVIPEQRTPSRAEKQDYSPLKKYVNL
PLPGVSSAKMMHDFGVSSMHSVIMDLPLSLDPLNMVKGKPVVEYDPTKPSRFGVFP
RHSPDQVRWFETKACCIFHTANTWDDMDQQGQVKSVHLLACRLTSGSTVFSAGNIA
GPVPRNSMVQEKKKGMPFFSKYDNDFERQVVRPGDEQQSYFDDPSAAYSDDIEAAP
LLREPTQRGQHIDEAVPARTPGSEVFELPEEIWEEWTDSEEDQCRLYHYNFSLESGRI
TSEYALARIPFEFPSVHPSFEMKRARYAFGCSTSCASFGAALGKSAKIDILVKIDAQRL
IAEGERQLAAGLLQPVTDCVDKRSLSQILDSNDPNDTIQAFHMPHGWFAQEPRFVPR
HNSTSEDDGFLLTYAFNEEQLTAEGEVPSDDDATLRSKSELWIIDAKDMRTVIGKVQ
LPQRVPYGLHGTWFSEDEVQGQRAVETVRSTAQALSKRDTGGVRMKIRDSVEKVLA
SEQ ID NO: 138 (Sphaerulina musiva SO2202 (XP_016756175.1)
MYLHHNDNEDDHDRDTRFTTTSLAGQKRKRACSTNILPTPQAKHPYLSGNFAPIQQT
LPLTPCAFTGRIPEELADGEYVRNGSNPVSNEDLGRDSHWFDGDGMLSGVSFTRDEQ
TGAVQPEFVNQFVLTDLYLNTLGSERLRVPILPSIATLVSPVASFVWVTLRILRTIVLV
VLSHLPGSRQKIKKISVANTNVVFHDGRALATCESGPPMRIQLPGLETVGWYNGASA
EGEMVDGVEGKEKEKVLGEDGGLLSFMREWTTAHPKVDPVSKEMLLYHASFAPPY
VQYSVIPQTPAAGMARSQKLLNVGVPKVSGAKMMHDFGVSSGHTVIMDLPLTLDPI
NQLKGLPTVHYDSSKPSRFGIFPRHSPELTKWFETDGCCIFHTANSWDSVNSLGHVSS
VNMLACRLTSATLIYAAGNMKPPPVKPLVSVTSKKNKRMPFFSKYDPGTDHTNFDP
TEPMSEKEPLLRIGERYTSIYEDERDPLAEDQCRLYYYSFDLATTSITHQWALSAVPFE
FPSVHPTREMQEARYVYGCSTTTTDFGAALGKATKIDAIVKIDAHSLIARGRASPPKS
VTGVVDSRTMAQVLESSDPQDPIQVFRMPPGWYAQEPRFVPAASSHSREEDEEEEEE
EEEEDNGYLLFYAFDETQLTAAGDVPPDDDSTRRATSELWIVNAKNMTEVIGRVQLP
QRVPYGLHGCWFSAEQIQSQRPVECIRTTVKALEERESRGLWMGVRDLVERWVG
SEQ ID NO: 139 (Filobasidium floriforme) (KAG7562916.1)
MTLFPLGKKARRRSSIHPYLAGNFAPVLEEYISHPCEIIEGVVPEELHGSQYVRNGGN
PVNPPDEGRNYHWFDGDGMLHGVFFPKATVAGESPSPPLYTNRHVSTPILSLSLMLL
RAPLPSISLLISPLTSLHRILGAIVTTFLLAFEAGVGTLSVVNTNVIWWGSGLGSEAEEE
RSKEGADGVIKKTGRLLGTCESGPPMEIRVPELETVEWDRLEDEQGESLREKYAKW
DPRGWRLARLQEDWMTAHPRVDPVNGDLVFYQCTMFEKPYLRYSVIDRHGKHRT
WKMGIDIGKAKMMHDFACSRKFTIFLNLPLTLSPFNMLSWPPRPMIHFDRNLPSEFII
MPRRTDASTKPIRFIDPEPSMIFHTANAWDDQCEKSGDAVIDMVACRFKSAKLVYAA
GAVAIPAEEVRAGFDDVVRLHYYRFLFESDGAHRISHNFPLSEIPFEFPATHPSKSMSK
ARFVYGCSMREGGFDERLGGAAKVDVLVKVDVIALSEQGRLRGEGKAKAVDQRTV
LQILEDHKNGRHDGIQVFVMPSGWFAQESRFVPRNIEGADEDDGWLLTYVYDEQYI
DAFGRASNAPGSGSELWIIDAKTMWKGQSSVVCRLKLPQRVPYGLHGTWIPGAHVD
MQRDLEHPITPQPTLVHKIMASRFRHFVDIFTARPRSRSRTLAERMAFWVLFLGSWIA
LGQAVLDISNDQDMRQGAVAWLSSHKSSLTRYLEPVSGLQLLGGLGIGGVLLVLLG
GSIS
SEQ ID NO: 140 (Mixia osmundae IAM 14324) (XP_014567302.1)
MGARDWRSLRKGKAREDEKRSSHPYLIGNFRPVEKEHHLTPCTVVEGSIPRELWGG
QYIRNGGNPAFPPKEKTAYHWFDGDGMLHGVHFRISTTADGVHSVEPHYTNRYIQT
DVFLASKTRGSTILPSISSLLISPSDSLGTKAALVPMLIRTVAIAYLSSLTRLSVTNTAV
YFHNRRLLATCESGPPMRIALPELTTVGWYVHPDENGEGGLGRGELTVGAKEWIGA
GRFAGAMLEEWQTGHPKLDPISGEFLTYGYNIFARPYVTYSVIGPHSEHRIFKQPVEIP
TAKMMHDWGVSRKHTIIMDLPLTMSPFNIAKGLKPMVYFNRKLKSRFGIMPRYFSG
QQSDIRWYETDPCLIFHTANAFDETDEAGAVNAVSLLACRFATAKLVHAAGGRPAP
AQEEAIAAALPDGDIVRLTYYRFDLSTSKNTITHEFALSNIPYEFPVANPAAVMRETQ
YLYGCTMRRGRFDEAMRAAKIDSFVKFDSRALVAKGKAQGFTRSGIVDTRSVMDVI
EQQSTYGPDHVPTDPVRIYALPAHHYAQEPAFVARQDASAEDDGYLLFYVYDERQL
DSLGSASDDTKGELWIIDSMKIGRSATHEDALVARISVPQRVPYGLHCNFISQAQIAS
QRREEWQAPKPVLTAAQIRAARTTEAIDVLLGEQDIGQRRDSLARLELAWLLYLSIA
GWLVLRSAPPHSSHVLLVAMIRSARDVCMTLLAARLFFIFQPRFAVLRHIFSAKHRTP
ASP
SEQ ID NO: 141 (Violaceomyces palustris) (PWN51250.1)
MAWLKRAQAPRQLQPYQPSPTNSEKVQDECLKRARAPPTSLTARPPLGRRESTSKRS
PTHPYLSGNFAPVSTEYPLTDCLYQGTIPSELAGCQYVRNGGNPMANSDADRDAHW
FDADGMLAGVLFRRLPDGSIQPSFLNRYILTDLILSTPDQLRRPILPSIATVNPGTSLL
WLLVQILRSFLLIMLSWLPGLGLRSEHKVRRISVANTSIYWHDGKAMAGCESGPPIRII
LPGLETGDWWTGEDQDGGGEADRVEDNQHLHEVDKGWGSRIPVVGMLREFTTAHP
KVDPVTGELLLYHMTFVPPHLRISVFPGSSVAKDGAERRVKAIKGVAVPGLSQPKM
MHDFAASSSQTVVLDLPLSLDPVNVLKGRPILHYDPTKASRFGVFPRRSPASVKWYE
SPEACCVYHTANCWDEYQDENEGEMGKRSSRLAAVNMLGCRLNSATLVYSAGNLV
PPNHALPPGNAPEKCQLYYWRFSDPPSDSSSAEERDGEATISHEFALSEVPFEFPSMN
EAYSMSEARFVYGTSLKDGTFDAGLGKAAKIDCLVKMDVGRLIQKGRRLWSAGQL
ERGGSVDRRTVEMILEDQRCGITNDAIRIFEMPSGWYAQEATFVARRKDLGEGAEAE
EDDGWLLCYVFDENSGLHPTTGDVLPGATSELWIIDAKRMDEVVCRIKLPQRVPYGL
HGTLFSEEQIASQRPIEAERVRSWARSINKQDPMRRYGVKERSWTNLVEAFLADPIR
VMAWEVKRGLEGLIA
SEQ ID NO: 142 (Testicularia cyperi) (PWZ01013.1)
MASSTSGPKRLRPGSLKRQGRQLSRGPTLEIPEPFATALCGTSSSASESASSAFPSSSK
VKLVPPRPTLTSKKEYIHPYLSGNFAPVTTECPLTDCLWAGQIPNELAGCQYVRNGG
NPLANSQLDRDAHWFDADGMLAGVLFRRREDGSLQPSFLNRFILTDLLLSTPEHSQL
PYLPSIATLVNPHTSFLRILSQILRSFLLAILTWLPGLGLRSDEKLRRISVANTSVFWHD
GKAMAGCESGPPMRIMLPGLETAGWYTGKDDDNDPSSKGFGDSSLPMLGMLREFT
TAHPRIDPQTGELLLYHMCFEPPFLRVSVIPSSTAEKAPVLSMKARANAKVLKGSPVR
GLQQPKMMHDFGATSTHTVILDLPLSLDMMNLVKGKPILHYDPSKPSRFGILPRYEP
DQVRWFDSPEACCVYHTANSWNDDRKIGAESCVTSAAEEPAAVNMLGCRLNSATL
VYSAGNLLPPSHVLPPPDCPEKCQLYYWRFDLESNKISHEFALSDVPFEFPSINEDYS
MKEARYVYGTSMRDGLAKIDALVKVDVHTLIRRGKQKWAQGKLERGDSVDTQEIL
ESQRCGDTACEGEEAIQIFEMPRGWYAQETTFVPRDIVNAAEDDGWLICYVFDEGQG
LHPSTGEVLPGATSELWIIDAKGMKDVVCRIKLPQRVPYGLHGTLFTEEQIQSQKPID
PSKIRSWASSVNNMDPFSSSSLGSSFVGTIGKKSSGAFPSSREAYNAFLRDPIRIGAWW
IKRNIEFLLA
SEQ ID NO: 143 (Friedmanniomyces endolithicus) (TKA42309.1)
MFEPPLPLAGHKRKRASSHLLQETPQPRHPYLTGNFAPIQQTLPLTPCTYTGTIPVELA
GGQYVRNGSNPVSNEDLGRDAHWFDGDGMLAGVLFRQDEENGEIQPEFVNRYILTD
LYLSTLSSPRLRVPILPSIATLVNPLYSLFYVTLRILRTVLLVILSFLPGSKQKINKISVA
NTNIVYHDGRALATCESGPPMRIQLPELETVGWYNGAWAQGEEDGDGVLDKEEMR
ELHGKKLGEDSAFALLGWIREWTTAHPKVDPVTKEMLMFHSSFAPPYVQYSIIPQQQ
HTPNPNTPAVSYEHTTQPRLEKLLNATVPGVAKAKMMHDFGVSLSHTVIMDLPLSL
DPVNQLRGLPPVTYDSSQPSRFGVFPRRQPDDVRWFETDASCIFHTANTWDTSEVDE
AGNTTTTEVNMLACRLTSATLIYASGNIAAPVERKPKVVLAETKKKRRMPFFSKYDD
AESTVYERAALLESPDEDEEKEPFVHINPGPSPSPFTAPDETLNEDSPSWEEDQCRLY
YYAFDLSSPATTNRIAHQWALTTIPFEFPSVRPDREMSAARYIYGCSTSSTSFGSALGK
ATKIDVLVKIDALGLVERGRASPPRSVGGSVDTRSMAAILASAAAEDPVKGFQMPEG
WFAQEPRFVPKRDGVGEDDGWLLFYAFDEGQLLPSGDVPGEDGGVGGEGRAKSEL
WVLSTRDMKTVVARVRLPQRVPYGLHGSWFDGEMIRGQRGVEGTPRTVGSVRGGE
TGGGGVWGASRRWVERMLG
SEQ ID NO: 144 (Pseudozyma flocculosa PF-1) (XP_007876729.1)
MASPGKPPRHAKPPLIHLPTPPGLSFSSTTEPPVIPDGDAIPRRPVLERSKSSKKCSVHP
YLSGNYAPVTLEYPLTDCLVSGQIPAELAGCQYVRNGGNPMANTETDRDAHWFDA
DGMLAGVLFRTLPDGTIQPSFLNRYILTDLVLSTPERSRYPFLPSIATLVNPRQSLISVL
LAILRTVLLAFLTWLPGLGLKRDQKLKRIGAANTSIYWHDGKAMAGCESGPPMRIM
LPGLETAGWWTGEEDPEEQTQDAASTAGPVSKEEKGRKGKGWGSGPPILGMLREFT
TAHPRVDPISGELLLYHMCFEPPFLRVTVIPATQKGSQPNTRKLLGVPVRGLSQPKM
MHDFGATRTRTVILDLPLSLDMMNMVKGRPILDYDPTKPSRFGIFPRHQPDEVRWYE
SPEACCVFHTANSWDGEDDSVNMLACRLNSATLVYSAGNLLPPSHVLPPANCPEKC
QLYYWRFQSEPEEVAEVAASRSRSSKGKAEGNRVNVISHEFALSDIPFEFPSINEERGL
QEARYIYGTSLKDGTFDAGLAKAAKIDCLVKVDAERLIRRGKQLWSQGKLGRGESV
DTRTVVEVLAQQRERAPGSPPDTIQVFEMPRGWYAQEATLVPRARKAGQAELAEDD
GWLMFYAFDERTGLHPTSGEVLPEATSELWIIDAKSMTDVVCRIKLPQRVPYGLHGS
LFNEEQIASQRPVPASQIRTWADSINNVDPFSLSSTSKSFAGAIDLKLSKDSTRSSAEM
YDAFLQRPIHVSAWWLKRTIESWIA
SEQ ID NO: 145 (Moesziomyces antarcticus) (XP_014658716.1)
MVVSQHAMHRSGSLKRPTGQKPTAPTISIPQPPALSEGPTYPPSDYLADSAEAGPSRP
KAIPRRPVLSRRRTSKKDYVHPYLSGNFAPVTTECPLTDCLYEGTIPEEFAGSQYVRN
GGNPLANSELDRDAHWFDADGMLAGVLFRRTPDGSIQPCFLNRFILTDLLLSTPEHS
RLPYLPSIATLVNPHVGLLWLMLGILRTFVLTVLTWLPGLGLRSDQKLRRISVANTSV
FWHDGKAMAGCESGPPMRIMLPGLETAGWYTGEDEEPSTVDKAAEKKRGKASEGF
GGGPPIVSMLREFTTAHPKVDPHTQELLLYHMCFEPPYLRVSVIAPAEVVAKKMPPQ
AKTIKGQAVRGLKQPKMMHDFGATPTHTVIIDVPLSLDLMNLVRGRPILHYDPAQPI
RFGVMPRYAPEQVRWFESAEACCVYHTANSWNDDGKFGASRTELVEPTEAGRSEPT
GINMLGCRLNSATLVYSAGNLLPPSHVLPPPNCPEKCQLYYWRFDLEDVDAERISHE
FALSEVPFEFPTINEEYAMRDARYVYGTSMRDGTFDAGLGKAAKIDALVKIDAQTLI
RRGKALWSQGRLGPGDSVDTRSVEEILAAQRDGSGAPDDAIRIFEMPRGWYAQETT
FVPRRSGAGPHEEDDGWLVCYVFDEAAGLHPSTGEVLPGATSELWIIDAREMKTVV
ARVKLPQRVPYGLHGTLFTEDQIAQQNPIDPAKIRSWAQSVTHADPFSASQLGSTLCT
APGKAASSKFSSAKHVYAAFLADPLHIGAWWIKRKIELLIA
SEQ ID NO: 146 (Sporisorium scitamineum) (CDU24508.1)
MVAADRDNRRRSSSMRKSNSQQHYVPIISISSPPPPAEEYAYPPSSSTTNLLIDEKSQA
EAGPSHAITSTAQPSHIPRRPVLSRRKTSKKEYVHPYLSGNFAPVTTECPLTDCLYEGT
IPQEFAGSQYVRNGGNPLANSDLDRDAHWFDADGMLAGVLFRRIVDGTIQPCFLNR
FILTDLLLSTPDHSRLPYLPSIATLVNPHTSIFWLLCEIIRTFVLTILTWLPGLGLTSDQK
LKRISVANTSVFWHDGKAMAGCESGPPMRIMLPGLKTVGWYTGEDEEEGEDEKHR
DADDRKTKSKGFGGGPPIISMLREFTTAHPKIDPKTQELLLYHMCFEAPYLRVSVIPP
ARSKRMHLPAHATTIKGKPVRGLKQPKMMHDFGATPTQTIILDVPLSLDMLNLVRG
KPILHYDPSQPSRFGVFPRYAPEQVRWFESPEACCVYHTANSWDDDGKFASSHVHLS
DANTTRGVNMLGCRLNSATLVYSAGNLLPPSHVLPPPNCSEKCQLYYWRFDLEHED
ANRISHEFALSDVPFEFPAINEEYLMQQARYVYGTSMHDGTFDAGLGKAAKIDALVK
VDAETLIRKGKAMWSQGRLKAGESVDTRTVEEVLAAQRDGSASAEDAIKIFEMPRG
WYAQETTFVPRRSGAQQEEDDGWLVCYVFDEAMGLHPSTGEVLPGATSELWIIDAK
DMKDVVCRVKLPQRVPYGLHGTLFTEEQIASQRPIDPSQVRSWASSVNLADPFSSSSL
GSTVYTASGKAAIGKFKDGKEVYAAFVKDPNRIGAWWIKRKIELFIA
SEQ ID NO: 147 (Ilumatobacter sp.) (TVR27654.1)
MTATALDQPPVRERPSRGFGAVVAITALIAAVGALTTDVPTSISIGVVVVVVALLGLP
HGAVDHLVEAAISGHDPTTRTSRLRFHVWYLGAMAGYGLVWLVAPTVALAGFLLV
SIHHFGQSDLAHLRLPGASQLAVQWSRGLFVIGLPLVAHLAVVSPVIGRLGGGDPAS
WSWLADRWALWSAAIVVQHVVVGAVVARRCDDRAAVRREAVTVAVLGALFVSV
DPLIGFAVYFGLWHSLAHLRVLAEVLGGASSADGRRTLPLRRFVALVAPRAVLSALG
LIGLVAAMYATGRPDLVLPAVFVLVSVLTVPHLVVVERLWRHRTSPSRSR
SEQ ID NO: 148 (Balneolaceae bacterium) (MBL6916485.1)
MATHAKTNNEIIQLQVFVGLAIIGLNFFLYEWFEAIRYYALAASVLIIGMPHGALDHK
IYFKALHKEETIKTQIVFYAGYLLVVAVYAWLWYVQPFIGFVLFMLFTLYHFGQSDT
ERLPLNGFLKQVFILARGLSIAGLILFGSDPFYTSRIVESVTGISLISIVYNFTTTEDLRLF
FALMYPSVFLFGSLVSKQVPLKSWLSLDALVVPALFSLVDPIFAFALYFGLWHGYNH
TKTMLDFLSSKNEQIGFGWFYKETALFSAISYIGLVLIYFLVEAFGDEALLVAILFTLIS
VITLPHMLVVEQMYRAFDKTDSDQST
SEQ ID NO: 149 (Uncultured marine bacterium 66A03) (Q4PNI0)
MGLMLIDWCALALVVFIGLPHGALDAAISFSMISSAKRIARLAGILLIYLLLATAFFLI
WYQLPAFSLLIFLLISIIHFGMADFNASPSKLKWPHIIAHGGVVTVWLPLIQKNEVTKL
FSILTNGPTPILWDILLIFFLCWSIGVCLHTYETLRSKHYNIAFELIGLIFLAWYAPPLVT
FATYFCFIHSRRHFSFVWKQLQHMSSKKMMIGSAIILSCTSWLIGGGIYFFLNSKMIAS
EAALQTVFIGLAALTVPHMILIDFIFRPHSSRIKIKN
SEQ ID NO: 150 (McCrtI; A0A168PH23; Mucor circinelloides)
MSKKHIVIIGAGVGGTATAARLAREGFKVTVVEKNDFGGGRCSLIHHQGHRFDQGPS
LYLMPKYFEDAFADLDERIQDHLELLRCDNNYKVHFDDGESIQLSSDLTRMKAELDR
VEGPLGFGRFLDFMKETHIHYESGTLIALKKNFESIWDLIRIKYAPEIFRLHLFGKIYDR
ASKYFKTKKMRMAFTFQTMYMGMSPYDAPAVYSLLQYTEFAEGIWYPRGGFNMV
VQKLEAIAKQKYDAEFIYNAPVAKINTDDATKQVTGVTLENGHIIDADAVVCNADL
VYAYHNLLPPCRWTQNTLASKKLTSSSISFYWSMSTKVPQLDVHNIFLAEAYQESFD
EIFKDFGLPSEASFYVNVPSRIDPSAAPDGKDSVIVLVPIGHMKSKTGDASTENYPAM
VDKARKMVLAVIERRLGMSNFADLIEHEQVNDPAVWQSKFNLWRGSILGLSHDVLQ
VLWFRPSTKDSTGRYDNLFFVGASTHPGTGVPIVLAGSKLTSDQVVKSFGKTPKPRKI
EMENTQAPLEEPDAESTFPVWFWLRAAFWVMFMFFYFFPQSNGQTPASFINNLLPEV
FRVHNSNVI
SEQ ID NO: 151 (Sp.CrtI; A0A0K0QVD9; Sporidiobolus pararoseus)
MSNTEKAYGNKNGHASGRPKAIIVGAGIGGCASAARLTQGGFDVTVLEKNDFAGGR
CSTFEGAPNYRFDQGPSLYLLPKLIAESFKDLGTSLDQEGIKLVKCEPNYRIVFPDKES
VEMSTDLSRMKKEVERWEGQDGFEGFLGFMKEAHVHYELSYEHVLHRNYTTLASII
RPTLLTQLRLLISVTSTVYTRASRYFKTERMRRAFTFASMYLGMSPFDALGAYNLLQ
YTEHCEGILYPLGGFSIVPKTLAKIAERNGANFRFNTPVERVIVENGQAKGVITESGEE
LRADVVLVNADLVWAMGHLYKETSYSKRLEEKPVSCSSISFYWSMNRKIPQLNSHTI
FLAEEYRESFDSIFTEHKIPHEPSFYVNVPSRHDASAAPEGKDSVIVLVPVGHISPALPT
ASDWNRIVDETREKIIGEIERRLDIKDLKKDIINEVVNTPITWSEKFNLHRGSILGLSHS
FFNVLSFRPKTRHPSVKNAYFVGASAHPGTGVPIVLAGARLATTQILQDYNLPIPASW
EVSSAELATASAKTDSNGLILLALLIALICALVTYARN
SEQ ID NO: 152 (Nc.CrtI; P21334; Neurospora crassa)
MAETQRPRSAIIVGAGAGGIAVAARLAKAGVDVTVLEKNDFTGGRCSLIHTKAGYRF
DQGPSLLLLPGLFRETFEDLGTTLEQEDVELLQCFPNYNIWFSDGKRFSPTTDNATMK
VEIEKWEGPDGFRRYLSWLAEGHQHYETSLRHVLHRNFKSILELADPRLVVTLLMAL
HPFESIWHRAGRYFKTDRMQRVFTFATMYMGMSPFDAPATYSLLQYSELAEGIWYP
RGGFHKVLDALVKIGERMGVKYRLNTGVSQVLTDGGKNGKKPKATGVQLENGEVL
NADLVVVNADLVYTYNNLLPKEIGGIKKYANKLNNRKASCSSISFYWSLSGMAKEL
ETHNIFLAEEYKESFDAIFERQALPDDPSFYIHVPSRVDPSAAPPDRDAVIALVPVGHL
LQNGQPELDWPTLVSKARAGVLATIQARTGLSLSPLITEEIVNTPYTWETKFNLSKGA
ILGLAHDFFNVLAFRPRTKAQGMDNAYFVGASTHPGTGVPIVLAGAKITAEQILEETF
PKNTKVPWTTNEERNSERMRKEMDEKITEEGIIMRSNSSKPGRRGSDAFEGAMEVV
NLLSQRAFPLLVALMGVLYFLLFVR
SEQ ID NO: 153 (Mc.CrtYB; Q9UUQ6; Mucor circinelloides)
MLLTYMEVHLYYTLPVLGVLSWLSRPYYTATDALKFKFLTLVAFTTASAWDNYIVY
HKAWSYCPTCVTAVIGYVPLEEYMFFIIMTLLTVAFTNLVMRWHLHSFFIRPETPVM
QSVLVRLVPITALLITAYKAWHLAVPGKPLFYGSCILWYACPVLALLWFGAGEYMM
RRPLAVLVSIALPTLFLCWVDVVAIGAGTWDISLATSTGKFVVPHLPVEEFMFFALIN
TVLVFGTCAIDRTMAILHLFKNKSPYQRPYQHSKSFLHQILEMTWAFCLPDQVLHSD
TFHDLSVSWDILRKASKSFYTASAVFPGDVRQELGVLYAFCRATDDLCDNEQVPVQ
TRKEQLILTHQFVSDLFGQKTSAPTAIDWDFYNDQLPASCISAFKSFTRLRHVLEAGA
IKELLDGYKWDLERRSIRDQEDLRYYSACVASSVGEMCTRIILAHADKPASRQQTQW
IIQRAREMGLVLQYTNIARDIVTDSEELGRCYLPQDWLTEKEVALIQGGLAREIGEER
LLSLSHRLIYQADELMVVANKGIDKLPSHCQGGVRAACNVYASIGTKLKSYKHHYPS
RAHVGNSKRVEIALLSVYNLYTAPIATSSTTHCRQGKMRNLNTI
SEQ ID NO: 154 (Mc.CrtYB; Q9UUQ6 Y27R; synthetic)
MLLTYMEVHLYYTLPVLGVLSWLSRPRYTATDALKFKFLTLVAFTTASAWDNYIVY
HKAWSYCPTCVTAVIGYVPLEEYMFFIIMTLLTVAFTNLVMRWHLHSFFIRPETPVM
QSVLVRLVPITALLITAYKAWHLAVPGKPLFYGSCILWYACPVLALLWFGAGEYMM
RRPLAVLVSIALPTLFLCWVDVVAIGAGTWDISLATSTGKFVVPHLPVEEFMFFALIN
TVLVFGTCAIDRTMAILHLFKNKSPYQRPYQHSKSFLHQILEMTWAFCLPDQVLHSD
TFHDLSVSWDILRKASKSFYTASAVFPGDVRQELGVLYAFCRATDDLCDNEQVPVQ
TRKEQLILTHQFVSDLFGQKTSAPTAIDWDFYNDQLPASCISAFKSFTRLRHVLEAGA
IKELLDGYKWDLERRSIRDQEDLRYYSACVASSVGEMCTRIILAHADKPASRQQTQW
IIQRAREMGLVLQYTNIARDIVTDSEELGRCYLPQDWLTEKEVALIQGGLAREIGEER
LLSLSHRLIYQADELMVVANKGIDKLPSHCQGGVRAACNVYASIGTKLKSYKHHYPS
RAHVGNSKRVEIALLSVYNLYTAPIATSSTTHCRQGKMRNLNTI
SEQ ID NO: 155 (Pb.CrtYB; Q9P854; Phycomyces blakesleeanus)
MLTYMEVHLYFTLPVLALLAFLYKPFFTTKDRFKYIFLCTVAFATASPWDNYIVYHK
AWSYCPECVTAVIGYVPLEEYMFFIIMTLITVTFTSLTMRWTLPSFFIRPETPVFQSVC
VRYIPIVGFLTIAAKAWASSIPDSHPFYGACILWYVCPVLALLWIGSGEYMLRRWKA
VLFSIAVPTIFLCWVDQYAIARGTWDISRRTSTGIMVLPSLPLEEFLFFLLIDTVLVFAS
CATDRAHAIVHIYITPMNHNKVSTWYMDFFYLCWAFLQTDQALSGETLSDLDATWR
ILREASASFYTASSVFSFEARQDLGVLYGFCRATDDLADNNDVSVPDRKKQLELVRG
FVRQMFDSKHGHPDIDWTQYSGSIPDSFIAAFRSFTRLRDVLEIKAVEELLDGYTFDL
EQREVKNEDDLVYYSACVASSVGEMCTRVLMASEPGGNRTMLKWTVERARDMGL
ALQLTNIARDIVTDSKQLGRSYVPRDWLTSQESALLKAGKARELGDERLRQIALKMV
YTADDLNLMASRAIDYLPPSSRCGVRAACNVYTAIGVSLHKANGYPDRAHLTKLER
MKVTFRCVYGFRKGHQGVQGDRGKSQAFTVI
SEQ ID NO: 156 (Nc.CrtYB; P37295; Neurospora crassa)
MYDYAFVHLKFTVPAAVLLTAIAYPILNRIHLIQTGFLVVVAFTAALPWDAYLIKHK
VWSYPPEAIVGPRLLGIPFEELFFFVIQTYITALVYILFNKPVLHALHLNNQQNPPAWM
RVVKVTGQVVLVALSVWGWNAAQVHQETSYLGLILVWACPFLLAIWTLAGRFILSL
PWYATVLPMFLPTFYLWAVDEFALHRGTWSIGSGTKLDFCLFGKLDIEEATFFLVTN
MLIVGGMAAFDQYLAVIYAFPTLFPKVNRYPTTHMLLQSRLINTSRYDLERIEGLREA
VERLRLKSRSFYLANSLFSGRLRIDLILLYSFCRLADDLVDDAKSRREVLSWTAKLNH
FLDLHYKDADATEDPKKKAERIDAYIKTAFPPCAYQALHLLPTHILPPKPLYDLIKGF
EMDSQFTFHGTSDSTDLQYPIADDKDLENYAIYVAGTVGELCIALIIYHCLPDMSDTQ
KRELETAACRMGIALQYVNIARDIVVDARIGRVYLPTTWLKKEGLTHKMVLENPEG
PEVIERMRRRLLENAFELYGGARPEMQRIPSEARGPMIGAVENYMAIGRVLRERKEG
TVFVRMEGRATVPKRRRLSTLLRALYEQ
SEQ ID NO: 157 (Farnesyl pyrophosphate synthase P08524)
MASEKEIRRERFLNVFPKLVEELNASLLAYGMPKEACDWYAHSLNYNTPGGKLNRG
LSVVDTYAILSNKTVEQLGQEEYEKVAILGWCIELLQAYFLVADDMMDKSITRRGQP
CWYKVPEVGEIAINDAFMLEAAIYKLLKSHFRNEKYYIDITELFHEVTFQTELGQLMD
LITAPEDKVDLSKFSLKKHSFIVTFKTAYYSFYLPVALAMYVAGITDEKDLKQARDV
LIPLGEYFQIQDDYLDCFGTPEQIGKIGTDIQDNKCSWVINKALELASAEQRKTLDEN
YGKKDSVAEAKCKKIFNDLKIEQLYHEYEESIAKDLKAKISQVDESRGFKADVLTAF
LNKVYKRSK

| SEQ | | | | |
|---|---|---|---|---|
| # | Name | Group | AA Sequence | Source Organism |
| SEQ | Mc. | Original | MSKKHIVIIGAGVGGTATAARLARE | Mucor |
| ID | CrtI_ | filing | GFKVTVVEKNDFGGGRCSLIHHQGH | circinelloides |
| NO: | A0A168PH23 | | RFDQGPSLYLMPKYFEDAFADLDER | |
| 150 | | | IQDHLELLRCDNNYKVHFDDGESIQ | |
| | | | LSSDLTRMKAELDRVEGPLGFGRFL | |
| | | | DEMKETHIHYESGTLIALKKNFESI | |
| | | | WDLIRIKYAPEIFRLHLFGKIYDRA | |
| | | | SKYFKTKKMRMAFTFQTMYMGMSPY | |
| | | | DAPAVYSLLQYTEFAEGIWYPRGGF | |
| | | | NMVVQKLEAIAKQKYDAEFIYNAPV | |
| | | | AKINTDDATKQVTGVTLENGHIIDA | |
| | | | DAVVCNADLVYAYHNLLPPCRWTQN | |
| | | | TLASKKLTSSSISFYWSMSTKVPQL | |
| | | | DVHNIFLAEAYQESFDEIFKDFGLP | |
| | | | SEASFYVNVPSRIDPSAAPDGKDSV | |
| | | | IVLVPIGHMKSKTGDASTENYPAMV | |
| | | | DKARKMVLAVIERRLGMSNFADLIE | |
| | | | HEQVNDPAVWQSKENLWRGSILGLS | |
| | | | HDVLQVLWFRPSTKDSTGRYDNLFF | |
| | | | VGASTHPGTGVPIVLAGSKLTSDQV | |
| | | | VKSFGKTPKPRKIEMENTQAPLEEP | |
| | | | DAESTFPVWFWLRAAFWVMEMFFYF | |
| | | | FPQSNGQTPASFINNLLPEVFRVHN | |
| | | | SNVI | |
| SEQ | Nc. | Original | MAETQRPRSAIIVGAGAGGIAVAAR | Neurospora |
| ID | CrtI_ | filing | LAKAGVDVTVLEKNDFTGGRCSLIH | crassa |
| NO; | P21334 | | TKAGYRFDQGPSLLLLPGLFRETFE | |
| 152 | | | DLGTTLEQEDVELLQCFPNYNIWES | |
| | | | DGKRFSPTTDNATMKVEIEKWEGPD | |
| | | | GFRRYLSWLAEGHQHYETSLRHVLH | |
| | | | RNFKSILELADPRLVVTLLMALHPF | |
| | | | ESIWHRAGRYFKTDRMQRVFTFATM | |
| | | | YMGMSPFDAPATYSLLQYSELAEGI | |
| | | | WYPRGGFHKVLDALVKIGERMGVKY | |
| | | | RLNTGVSQVLTDGGKNGKKPKATGV | |
| | | | QLENGEVLNADLVVVNADLVYTYNN | |
| | | | LLPKEIGGIKKYANKLNNRKASCSS | |
| | | | ISFYWSLSGMAKELETHNIFLAEEY | |
| | | | KESFDAIFERQALPDDPSFYIHVPS | |
| | | | RVDPSAAPPDRDAVIALVPVGHLLQ | |
| | | | NGQPELDWPTLVSKARAGVLATIQA | |
| | | | RTGLSLSPLITEEIVNTPYTWETKF | |
| | | | NLSKGAILGLAHDFFNVLAFRPRTK | |
| | | | AQGMDNAYFVGASTHPGTGVPIVLA | |
| | | | GAKITAEQILEETFPKNTKVPWTTN | |
| | | | EERNSERMRKEMDEKITEEGIIMRS | |
| | | | NSSKPGRRGSDAFEGAMEVVNLLSQ | |
| | | | RAFPLLVALMGVLYFLLFVR | |
| SEQ | Sp. | Original | MSNTEKAYGNKNGHASGRPKAIIVG | Sporidiobolus |
| ID | CrtI_ | filing | AGIGGCASAARLTQGGFDVTVLEKN | pararoseus |
| NO: | A0A0K0QVD9 | | DFAGGRCSTFEGAPNYRFDQGPSLY | |
| 151 | | | LLPKLIAESFKDLGTSLDQEGIKLV | |
| | | | KCEPNYRIVFPDKESVEMSTDLSRM | |
| | | | KKEVERWEGQDGFEGFLGFMKEAHV | |
| | | | HYELSYEHVLHRNYTTLASIIRPTL | |
| | | | LTQLRLLISVTSTVYTRASRYFKTE | |
| | | | RMRRAFTFASMYLGMSPFDALGAYN | |
| | | | LLQYTEHCEGILYPLGGFSIVPKTL | |
| | | | AKIAERNGANFRENTPVERVIVENG | |
| | | | QAKGVITESGEELRADVVLVNADLV | |
| | | | WAMGHLYKETSYSKRLEEKPVSCSS | |
| | | | ISFYWSMNRKIPQLNSHTIFLAEEY | |
| | | | RESFDSIFTEHKIPHEPSFYVNVPS | |
| | | | RHDASAAPEGKDSVIVLVPVGHISP | |
| | | | ALPTASDWNRIVDETREKIIGEIER | |
| | | | RLDIKDLKKDIINEVVNTPITWSEK | |
| | | | FNLHRGSILGLSHSFFNVLSFRPKT | |
| | | | RHPSVKNAYFVGASAHPGTGVPIVL | |
| | | | AGARLATTQILQDYNLPIPASWEVS | |
| | | | SAELATASAKTDSNGLILLALLIAL | |
| | | | ICALVTYARN | |
| SEQ | Xd. | Original | MGKEQDQDKPTAIIVGCGIGGIATA | Phaffia |
| ID | CrtI_ | filing | ARLAKEGFQVTVFEKNDYSGGRCSL | rhodozyma |
| NO: | O13506 | | IERDGYRFDQGPSLLLLPDLFKQTF | (Xanthophyllomyces |
| 11 | | | EDLGEKMEDWVDLIKCEPNYVCHFH | dendrorhous) |
| | | | DEETFTFSTDMALLKREVERFEGKD | |
| | | | GFDRFLSFIQEAHRHYELAVVHVLQ | |
| | | | KNFPGFAAFLRLQFIGQILALHPFE | |
| | | | SIWTRVCRYFKTDRLRRVFSFAVMY | |
| | | | MGQSPYSAPGTYSLLQYTELTEGIW | |
| | | | YPRGGFWQVPNTLLQIVKRNNPSAK | |
| | | | FNFNAPVSQVLLSPAKDRATGVRLE | |
| | | | SGEEHHADVVIVNADLVYASEHLIP | |
| | | | DDARNKIGQLGEVKRSWWADLVGGK | |
| | | | KLKGSCSSLSFYWSMDRIVDGLGGH | |
| | | | NIFLAEDFKGSEDTIFEELGLPADP | |
| | | | SFYVNVPSRIDPSAAPEGKDAIVIL | |
| | | | VPCGHIDASNPQDYNKLVARARKFV | |
| | | | IQTLSAKLGLPDFEKMIVAEKVHDA | |
| | | | PSWEKEFNLKDGSILGLAHNFMQVL | |
| | | | GFRPSTRHPKYDKLFFVGASTHPGT | |
| | | | GVPIVLAGAKLTANQVLESFDRSPA | |
| | | | PDPNMSLSVPYGKPLKSNGTGIDSQ | |
| | | | VQLKFMDLERWVYLLVLLIGAVIAR | |
| | | | SVGVLAF | |
| SEQ | Ak. | Expanded | MAKKSAIVVGAGVGGISTAARLARE | Aspergillus |
| ID | CrtI_ | Sequences | GFKVTVLEKHAFVGGRCSIIERDGY | kawachii |
| NO: | G7XI79 | | RFDQGPSLLLMREIFSRTFQDLGTS | |
| 158 | | | LSSEGVELLKCEPNYCIWFPDNDIV | |
| | | | ELSSDITRLKEEVIRHEGITGLSKL | |
| | | | LDFLKETGQYYNLTLDYIMNRDFPH | |
| | | | FLSLLQPNVLRALVAMRPWNTMAGI | |
| | | | VEQYFTSTKMKQAWTFASMYMGMSP | |
| | | | YQAPGTYSLLQYSETVDGIWYPRGG | |
| | | | FQKVLQALADVGRRLGVDYRLRAPV | |
| | | | DSVLLAPDQRSAKGVRLNSGEEIHA | |
| | | | DLVVINADLVYAYNNLLPESGYGRS | |
| | | | LQKRHTSCSSISFFWAFDTTFPQLK | |
| | | | SHNIFLAEKYRESFDSIFVDHSIPD | |
| | | | EPSFYLNVPSRIDPTAAPPGKDAVV | |
| | | | VLVPVGSLREPGTNDKKHWERIIPD | |
| | | | IRDLVIKTIEERTGLQNLRSKLLHE | |
| | | | TYETPHTWKDKFNLDRGAILGLSHS | |
| | | | FFNMLSFRPSLKHPTIDGLYFAGAS | |
| | | | THPGTGVPVCLAGSKLVCERILRDT | |
| | | | RLEQRMLFATAYIWVTFTLVVVLAT | |
| | | | ACVTLLYPSCLDSFLCLVQ | |
| SEQ | Ao. | Expanded | MSQKHIIIIGAGVGGVSTAAKLSRE | Apophysomyces |
| ID | CrtI_ | Sequences | GFQVTVLEKNNFVGGRCSFIHHDGH | ossiformis |
| NO: | A0A8H7BN47 | | RFDQGPSLYLMPKLFEEAFADVDER | |
| 159 | | | ITDHLELLRCQNNYKIYEDDGDAIQ | |
| | | | LSSDLSHMKAEMERIEGQDGEGNFL | |
| | | | KFMGETHVHYTRGVVLALKQNFESF | |
| | | | WDMVRLKHAPEIVRLHLFSKIYSRA | |
| | | | SRYFRTKKMRMAFTFQTMYMGMSPF | |
| | | | DAPAVYSLLQYTEFAEGIWYPRGGF | |
| | | | NMVAQKLEAIAAQKYGAKFIYEAPV | |
| | | | AKINTDAKRVTGVTLENGEVIEADA | |
| | | | VVCNADLVYAYHNLLPPCQWTQNHL | |
| | | | AKKKLTSSSISFYWSLKQVVPQLDV | |
| | | | HNIFLAEAYRESFDEIFQDYSLPSE | |
| | | | ISFYVNIPSRIDPTAAPPNKDTMVV | |
| | | | LVPVGHMRGLETSSEEKYYAAMVNR | |
| | | | ARRMVLEVLAQRLGLENFHDLIEHE | |
| | | | IVNDPTVWQSKFNLWRGSVLGLSHN | |
| | | | ILQVLWFRPSTKDSTGRFDNLFFVG | |
| | | | ASTHPGTGVPIVLAGSKLTADQVCR | |
| | | | HFGKVPTPSKLANQQKQRIYESEKQ | |
| | | | PVSWTWWYPLIFLFVLAFAFFVSFP | |
| | | | ENQSSETAASFINTCLPSMERVESA | |
| | | | SLQ | |
| SEQ | Ao. | Expanded | MSSIPIGILSRPESRQLIAPGAGAG | Aspergillus |
| ID | CrtI_ | Sequences | GIATAARLAQEGFHVKVVEQHGFIG | oryzae |
| NO: | I7ZZQ8 | | GRCSIISKDGYRFDQGPSLLLMREV | |
| 160 | | | FEETFQDLGTSLEQENVRLVKCEPN | |
| | | | YCVWFPDKDIIELSTNLTRLKAQIQ | |
| | | | HHEGPDGFSRFCAFLNEAGTHYNLS | |
| | | | LAHVLRKNFPGFLSLLRWDVLRSLI | |
| | | | SMHPWTSTYSRAARYFYSEKMRRVW | |
| | | | TFGSMYLGMSPYRAPGTYSLLQYIE | |
| | | | TVDGIWYPAGGFQRVLRALGDIGMR | |
| | | | SGVEYILNSPVKSVLLDDSNHVAKG | |
| | | | VLLEDGEELYADLVVINADLVYAYN | |
| | | | ELLPKTRRSHDLKKRPVSCSSISFF | |
| | | | WSFDEKLPHLRAHNIFLAEKYRESF | |
| | | | DAIFEDHRIPDEPSFYVNVPSKIDP | |
| | | | TAAPPGKEAVVVLVPVGHLTSEKGG | |
| | | | QLEEEKWDTLVSQTREIVLDTIEAR | |
| | | | TGLQDLRSRLVHEMVETPLSWEERF | |
| | | | NLDRGAILGLSHSFFNVLSFRPQIK | |
| | | | HPDIERLYFVGASTHPGTGVPVCLA | |
| | | | GSKLVTQQIVEDWNMGIRQKPRSGF | |
| | | | ILTLVMALLTLVVSFLWRH | |
| SEQ | Ar. | Expanded | MTPKAVVVGAGAGGVATAARLAKQG | Aspergillus |
| ID | CrtI_ | Sequences | FQVTVIEKNGFIGGRCSLITHNGYR | ruber |
| NO: | A0A017RZD5 | | FDQGPSLLLMPEVFHDTENQLGTSL | |
| 161 | | | EEEGIKLLKCEPNYRLWFTDNDSED | |
| | | | LSTDIASLKPQIEKYEGKEGFQNFL | |
| | | | QFMQESGRHHDLSLIHVLRRRFPSL | |
| | | | LSMLRPGFLLSVEKMHPFESIHGRL | |
| | | | SRYFRSEKMRRIFTFASMYLGMNPY | |
| | | | EAPGTYSLLQYTELADGIWYPAGGF | |
| | | | QKVLESIATIGKSFGVEYRLNSPVS | |
| | | | SILLSGDSKSATGVVLSSGERLDAD | |
| | | | IVVINADLVYAYQNLLPASKEACRL | |
| | | | SNRPASCSSISFFWSFDRVVPELKA | |
| | | | HNIFLAEKYEESFDAIFKYHKLPDE | |
| | | | PSFYVNVPSRIDRTAAPDEKDAVVV | |
| | | | LVPTGHLLENTESITEADWITIVSK | |
| | | | ARETAINTIEARTGVRGLRDILVHE | |
| | | | SIETPVSWKEKENLDRGAILGLSHS | |
| | | | FFNVLSFRPKNKHPDIARLYFVGAS | |
| | | | THPGTGVPVCLAGSKLVVDQVVEDW | |
| | | | NADKKSSLAGICLVIVSMLIVMVSL | |
| | | | MPFVTFCHHALHTVISS | |
| SEQ | Ar. | Expanded | MTPPKHIIVIGAGAGGTASAARLAR | Absidia repens |
| ID | CrtI_ | Sequences | QGFRVTVVEKNDENGGRCSLIYHNG | |
| NO: | A0A1X2IZQ9 | | YRFDQGPSLYLMPKLFEDTFADLDE | |
| 162 | | | KLDDHIDLLRCSNNYKVHFDDGDKI | |
| | | | HLTSDLTLMKTEMERVEGEHGFSRF | |
| | | | LDEMSETHVHYENGTLLAIKRNFAT | |
| | | | IWDMIKIKYAPEIFRLHLEDKIYNR | |
| | | | ASLYFKTKKMRMAFTFQTMYMGMSP | |
| | | | YDAPAVYSLLQYTEFAEGIWYPRGG | |
| | | | FNTVVQRLESIARDKYGAKFIYNAP | |
| | | | VARINTKDNQQQVTGVTLENGTVIE | |
| | | | ADAVVCNADLVYAYHHLLPPCPWTT | |
| | | | STLAKKKLTSSSISFYWSLSQTVPE | |
| | | | LDVHNIFLAENYKESFDEIFQDHSL | |
| | | | PSEPSFYVNLPSRIDPSAAPPNKDA | |
| | | | MIVLVPIGHMRSESGKKADQQEMDD | |
| | | | YEVMVERARTMVLTVLERRLGINNF | |
| | | | RHMIDHEIVNHPGIWQEKENLWRGS | |
| | | | VLGLSHDVLQVLWFRPSTKDSTGRY | |
| | | | NNLFFVGASTHPGTGVPIVLAGSKL | |
| | | | TSDQVCQHFNQIPMPRLQQQKMTET | |
| | | | TDGGVISNNKKKVFEYKPEMEYNNG | |
| | | | FILSNTLWYWCASLFLLTLTFFVAF | |
| | | | PTNHTNHTAASYINSSMPSIFRVGK | |
| | | | YECTMEDMCR | |
| SEQ | As. | Expanded | MSATMNGAGAGERTAIVIGSGFGGL | Aurantimonas |
| ID | CrtI_ | Sequences | ALAIRLQASGIQTTLLEKRDKPGGR | sp. SI85-9A1 |
| NO: | Q1YGX8 | | AYVYEDQGFIFDAGPTVITDPSALE | |
| 163 | | | ELFTAAGRTMADYVELLPVKPFYRL | |
| | | | CWEDGYSFDYANDQDELDRQIHAKN | |
| | | | PADIEGYRQFLAYSREVFREGYEKL | |
| | | | GTVPFLSFRDMVKAGPQLAKLQAWR | |
| | | | SVYSKVAQYIEDEQLRQAFSFHSLL | |
| | | | VGGNPFATSSIYALIHALEREWGVW | |
| | | | FPRGGTGALVRGLVRLFEDMGGRVE | |
| | | | LSAEAAQIEVVGDRAKAVVLKDGRR | |
| | | | FEADRIASNADIVHTYGTLMAGTRR | |
| | | | GEKEAKRLKARRFSMSLFVIYFGLK | |
| | | | RHRPELQHHTVCFGPRYRPLIDEIF | |
| | | | KGGELPGDFSLYLHSPCATDPSLAP | |
| | | | EGAGSYYVLAPVPHLGNTDIDWEIE | |
| | | | GPKYRDRILDYLEERYIPDLRADLV | |
| | | | TSRIFTPFDERDELNAHVGSAFSLD | |
| | | | PVLTQSAWFRPHNRDADIPNLYFVG | |
| | | | AGTHPGAGVPGVVGSAKATAGLMIA | |
| | | | DSRA | |
| SEQ | B1. | Expanded | MTERIGVIGGGAAGLATAALLAREG | Brevibacterium |
| ID | CrtI_ | Sequences | HSVDLFEQNSTLGGRIGSIEEDGYR | linens |
| NO: | Q9KK84 | | FDTGPSWYLMPEVYEHFFNLLGTTA | |
| 164 | | | ANELDLRTLDPAYTVFSPASQARRE | |
| | | | QSVTIPHGTAEIIDTFERIERGSGA | |
| | | | TLAKYLDSAKATKDLAIDYFLYNPF | |
| | | | TSPKSILRAELLTALPALARLLTTS | |
| | | | LERHASRSFHHPVLRQILQYPAIFL | |
| | | | GTDPRKAPALYHLMSALDLSDGVLY | |
| | | | PMGGFRTIVDALTRLVEESGVRVRT | |
| | | | DSTVTHISTVSESGRRRSANGIAWK | |
| | | | DQDGREHFHDCDIVVSAADLHHTET | |
| | | | QLLGEGERSYPESWWQKVTSGPGAV | |
| | | | LVFLGVEGEVPQLPHHSLFFTEDWS | |
| | | | TNFDDIFGSQQKISSPASAYVCKPS | |
| | | | HTDPNVAPFDCENLFILIPVPADAG | |
| | | | LGAGGDDGQGDPRIERAADQAIDQI | |
| | | | AEWADIPDLRDRIRFRRTLGPTDES | |
| | | | SNYNSWLGGMLGPAHTLRQSAMFRQ | |
| | | | QNASKKVDGLYYAGATTAPGVGVPM | |
| | | | CLISAELVLKHIRGDSSPGPLPVTD | |
| | | | TRE | |
| SEQ | Bo. | Expanded | MLDVSPLKAAVPAALGDRPHAVVIG | Bradyrhizobium |
| ID | CrtI_ | Sequences | SGFGGLAAAVRLGARGYRVTVFEQL | sp. (strain ORS |
| NO: | Q7BP90 | | DAPGGRAYVHRQDGFTLDAGPTIVT | 278) |
| 165 | | | APFLFEELWQLCGRRMSDDVTLKPI | |
| | | | SPFYRIREDDGTIFDCSADPAAMRA | |
| | | | EVARISPRDVDGYERFMMTSEAIFR | |
| | | | KGFEELGDVPFLSLEDMAKIAPAMI | |
| | | | KLQSFRSVYGLVAQHVLDERLRVIL | |
| | | | SFHPLLIGGNPFTTTSIYCLIAYLE | |
| | | | RRWGVHFAMGGTSALVSGLVGLIEG | |
| | | | QGGEVRCSAPVAEITTINGAATGVR | |
| | | | LTSGETIAADVVVSNGCAAWTYKNL | |
| | | | LPSVRRRRWTDARIDRARYSMSLFV | |
| | | | WYFGTKRRYEDVEHHTILLGPRYRE | |
| | | | LLDDIFRRKVLADDESLYLHRPTAT | |
| | | | DPSLAPNGCDAFYVLSPVPHQESGI | |
| | | | DWTVAAEPFRKRIETALSQTLLPDL | |
| | | | ASQIVTSRLLTPDDFEQRLSSYRGA | |
| | | | AFGLEPVLMQSAWFRPHNKSEEVER | |
| | | | LYLVGAGTHPGAGLPGVLSSARVLD | |
| | | | DLVPAPHELERR | |
| SEQ | Bo. | Expanded | MSQEMQNAKTAVVIGSGFGGLSLAI | Bradyrhizobium |
| ID | CrtI_ | Sequences | RLQSAGIATTLVEKRDKFGGRAYVY | sp. ORS278 |
| NO: | Q9KIX2 | | EQDGFTFDAGPTVITDPTCLQELFA | |
| 166 | | | LSGRKLENYVELMPVSPFYQLRWED | |
| | | | GATFDYVNDQAELERQIAAFCPADV | |
| | | | DGYRRFRSYSERLLEEGYVKLGHVP | |
| | | | FPDFRSMVRVAPQLVALQSYRSVYS | |
| | | | KVSQYVSDEHLRQAFSFHSLLVGGN | |
| | | | PFATSSIYALIHALERRWGVWFPRG | |
| | | | GTGALINKGLVQLFKDLGGEVTLST | |
| | | | SVSRIETANGRVSAVVAEDGRRFAA | |
| | | | DIVASNADVVHTYRDLLKDEPLARP | |
| | | | TAQSLMRKRFSMSLFVIYFGLRREH | |
| | | | PELKHHIILFGRRYRELINEIFKGP | |
| | | | ALPEDFSLYLHAPSVTDPSLAPQGC | |
| | | | STYYVLSPVPHLAAAPIDWSVEGPR | |
| | | | YRDRILDYLEARILPGLKSDLATCR | |
| | | | IFTPQDENTELNAHLGSAFSLEPIL | |
| | | | TQSAYFRAHNADDKIKGLYLVGAGT | |
| | | | HPGAGIPGVVGSAKATARVILEDQR | |
| | | | ELAEAQ | |
| SEQ | Bs. | Expanded | MRAAVIGSGFGGLSLAIRLQTAGIQ | Brevundimonas |
| ID | CrtI_ | Sequences | TTVFEARDLPGGRAYVYKDKGYTED | sp. SD212 |
| NO: | Q4W8B6 | | AGPTVITDPSALEELFEGAGRKLSD | |
| 167 | | | YVELLPVAPFYRLCWEDGDVFDYVN | |
| | | | GQDELDRQIVARNPADKEGYRRFLA | |
| | | | YSQDLLKEGYLKLGAVPFLDFASMV | |
| | | | KAAPELMRLQAWRSVYDKVAGYIQD | |
| | | | EHLRQAFSFHSLLVGGNPFATSSIY | |
| | | | ALIHALERRWGVWFPRGGTGALIQA | |
| | | | MVRLFQDLGGEIRLNSPVERITLAN | |
| | | | GRADGVVVGGQALAFDMVASNADVV | |
| | | | HTYQRLLGQEPRGRKEGARLASKRH | |
| | | | SMSLFVIYFGLKRVHPEVRHHTVLF | |
| | | | GPRYRELIGEIFKGPDLPQDFSLYL | |
| | | | HAPTRTDPSLAPEGCDAFYVLAPVP | |
| | | | HLASADIDWAVEGPRYRDRVLAYLE | |
| | | | QHYIPGLTAHLDTCRIFTPVDERDQ | |
| | | | LNAHQGSAFSLEPILTQSAYFRVHN | |
| | | | RDDQIPNLYFVGAGTHPGAGVPGVV | |
| | | | GSAKATAGLMIEDAGRTA | |
| SEQ | Bv. | Expanded | MMRAAVIGSGFGGLSLAIRLQSAGI | Brevundimonas |
| ID | CrtI_ | Sequences | QTTVFEARDLEGGRAYVFKDKGYTF | vesicularis |
| NO: | Q0H2D1 | | DAGPTVITDPSALEELFEASGRKLS | DC263 |
| 168 | | | DYVELLPVAPFYRLCWEDGDVFDYV | |
| | | | NDQDELDRQIVARNPADKEGYRKEL | |
| | | | AYSQDLLKEGYLKLGAVPELDFASM | |
| | | | VKAAPELMRLQAWRSVYDKVASYIQ | |
| | | | DEHLRQAFSFHSLLVGGNPFATSSI | |
| | | | YALIHALERRWGVWFPRGGTGALIQ | |
| | | | AMVRLLKDLGGEVRLNSPVERITMA | |
| | | | NGRATGVVVNGEALAFDMVASNADV | |
| | | | VHTYQRLLGQEPRGQKEGAKLASRR | |
| | | | HSMSLFVIYFGLKRVHPEVRHHTVL | |
| | | | FGPRYRELIAEIFKGPDLPDDESLY | |
| | | | LHAPTRTDPSLAPEGCDAFYVLAPV | |
| | | | PHLASADIDWAVEGPRYRDRILAYL | |
| | | | EERYIPGLTADLDTCRIFTPVDERD | |
| | | | QLNAHQGSAFSLEPILTQSAWFRVH | |
| | | | NRDDQIPNLYFVGAGTHPGAGVPGV | |
| | | | VGSAKATAALMIEDARQPA | |
| SEQ | Cc. | Expanded | MSEQKKHIIVIGAGVGGTATAARLA | Choanephora |
| ID | CrtI_ | Sequences | REGFRVTVVEKNDESGGRCSFIYHD | cucurbitarum |
| NO: | A0A1C70F9N | | GHRFDQGPSLYLMPKLFEDAFADLD | |
| 169 | | | ERIEDHLDLLRCDNNYKVHFDDGDA | |
| | | | VQLSSDLTKMKAELERIEGPLGFGK | |
| | | | FLDFMKETHVHYEQGVFIAIKRNFE | |
| | | | TIWDLIRLQYVPEIFRLHLFGKIYD | |
| | | | RASKYFQTKKMRMAFTFQTMYMGMS | |
| | | | PYDAPAVYSLLQYTEFAEGIWYPRG | |
| | | | GENMVVQKLEAIASEKYGAQFKYQS | |
| | | | PVAKINTADKDKRVTGVTLESGEVI | |
| | | | EADAVVCNADLVYAYHHLLPPCNWT | |
| | | | KKTLASKKLTSSSISFYWSMSTKVP | |
| | | | QLDVHNIFLAEAYKESFDEIFNDES | |
| | | | LPSEASFYVNVPSRIDSTAAPPNKD | |
| | | | SIIVLVPIGHMKSKTGNATEENYSE | |
| | | | LVNRARKMVLEIIERRLGVSNFSSL | |
| | | | IEHEEVNDPSVWQSKENLWRGSILG | |
| | | | LSHDVFQVLWFRPSTKDSTNRYDNL | |
| | | | FFVGASTHPGTGVPIVLAGSKLTSD | |
| | | | QVCQSFGQTPLPRKLQDNRKKYAPE | |
| | | | KPSQNGSHWIYYCLACYFVTFLFFY | |
| | | | FFPRDENTTPASFINQLLPNVFQAQ | |
| | | | NSNDIRI | |
| SEQ | Cn. | Expanded | MPSTSKRPTAIVIGSGVGGVSTAAR | Cercospora |
| ID | CrtI_ | Sequences | LARAGFHVTVLEKNNFTGGRCSLIH | nicotianae |
| NO: | P48537 | | HEGYRFDQGPSLLLLPGLFHRTFAE | |
| 170 | | | LGTSLEQEGVKLLKCEPNYMIHFSD | |
| | | | GEKFTLSSDLSVMKTEVEKWEGKEG | |
| | | | YTRYLEFLKESHGHYELSVREVLLR | |
| | | | NFEGLTAMLRPEFLRHLLQLHPFES | |
| | | | IWTRAGKYFWTERLRRVFTFGSMYM | |
| | | | GMSPFDAPGTYSLLQYTELAEGIWY | |
| | | | PVGGFHRVVEALVKIGEREGVDFRM | |
| | | | ETAVKKILLSEDGGVAKGVELEDGR | |
| | | | RLEADVVVNNSDLVYAYEKLLPIKT | |
| | | | PYAESLKGRPGSCSSISFYWALDRQ | |
| | | | VPELEAHNIFLADEYRESEDSIFKK | |
| | | | HLIPDEPSFYVNVPSRVDSTAAPEG | |
| | | | KDSVVVLVPVGHLLEEDRHASQAHQ | |
| | | | LSASRNGHISSASPPDQPGLTPTEK | |
| | | | QDWPAMISLARKTILSTIQSRTNVD | |
| | | | LTPLIIHESTNSPLSWKQTENLDRG | |
| | | | AILGLSHSFENVLCFRPTTRARKPG | |
| | | | AFDAQLLKFGVLGRAAEVIIDAFRG | |
| | | | RGKDIKGLYMVGASAHPGTGVPICL | |
| | | | AGGALVAEQICGDYGVDIPWKEEER | |
| | | | KGRDVGGKRKLDVLESPMWLDSWEQ | |
| | | | WVSVLIYLLVGIFAWLWMKER | |
| SEQ | Cp. | Expanded | MKSIVIGSGFGGIAAALRLRAKNHK | Candidatus |
| ID | CrtI_ | Sequences | VTLIEKHPDLGGRARVFKKNGFTED | Pelagibacter |
| NO: | Q4FPE5 | | AGPTVITAPYLIEELFTLENKKSQD | ubique HTCC1062 |
| 171 | | | YIKLTPLKTWYRFIYEDGGVFDYSG | |
| | | | NEDQMKKQIEKINKEDVKGYEQLVK | |
| | | | FTKKIFDKGFTELADVPFDKPLVMM | |
| | | | KQLPSLLKLKSYKSVYSLVSSYIKN | |
| | | | EKLRRMLSMHPLLVGGNPFTTTSIY | |
| | | | GLILYLEKKWGIHYSMGGTGNIING | |
| | | | LEKLMIEQEIEVIKGHEVTKIISDN | |
| | | | GKISGVKLDNQKEMLSNNVICNADP | |
| | | | PAVYEKLLSLNKTSPLFKWKQNRMQ | |
| | | | YSMGLFVYYFGTKKTYPDVEHHTIK | |
| | | | FGDKYKEHLSDIFDNKKLNDDISYY | |
| | | | LHRPSATDKSMAPEGNDCFYVLVPV | |
| | | | PNNQSKIDWKIEGEKMKNLVIDKME | |
| | | | KALLPNLRENIIEDFYLTPDYFENE | |
| | | | LNTKFGSGFSIQPKFTQSAYFRFHN | |
| | | | KSEIYDGLYFVGAGTHPGAGVPGVL | |
| | | | SSAKVLDKIL | |
| SEQ | Cs. | Expanded | MLMKSDAAAPRAAGKQDPQAPVALV | Rubrivivax |
| ID | CrtI_ | Sequences | VGSGFGGMAAAVRLAAKGYRVTVLE | gelatinosus S1 |
| NO: | Q840T3 | | KLDAPGGRAYVHRREGHVFDAGPTI | |
| 172 | | | VTVPYLFDELWALAGRKFSDDIELK | |
| | | | SLDPFYRIRFDDGDHFDYSGDPARM | |
| | | | RAEVRRISPSDAEGFEREMREADQC | |
| | | | YELGFRTLGDKAFDTVGDLIKAAPL | |
| | | | IIRLRGWRSLHQMVSSHLKHPKLRI | |
| | | | AMSLQSLLIGGNPFSVTSMYALVNA | |
| | | | LERQWGVHWAMGGTGELIRGLVDVF | |
| | | | EGMGGTMRLKAEVKRIEVDNGVATG | |
| | | | VTLADGERLPADIVVCNGDTGYLYK | |
| | | | NLVDARWRKHWTDARIERGHYSMGL | |
| | | | FVWYFGTDRRYEDVPHHMMVLGPRY | |
| | | | RELLDDIFRKKKLAGDESIYLHRPT | |
| | | | ATDPSMAPAGCDTFYALMPVPHLGS | |
| | | | GTDWTTQAEPYRQSVQEALERTVLP | |
| | | | GLGQHLRVSFCTTPLDFQHRLLSYK | |
| | | | GAGFGLEPLLLQSAYFRPHNRSEDV | |
| | | | KNLFMVGASTHPGAGVPGVIMSAKA | |
| | | | LESVLPDPATERR | |
| SEQ | Dt. | Expanded | MNQKAVVIGAGVGGTAVAARLAKQG | Dinothrombium |
| ID | CrtI_ | Sequences | FEVTVYEKNDFGGGRCSLINKNGHR | tinctorium |
| NO: | A0A443RCG3 | | FDQGPSLYLMPKIFEETFRDLGEDI | |
| 173 | | | NDHLELLKCESNYKVHFHDGNSIQL | |
| | | | TCDLPKMCAEIERFEGEKETTIENE | |
| | | | LSFLRECHIHYDHSVRLALKTNFPT | |
| | | | LSDLLRPKYLPEVFKMHLYDTVYNR | |
| | | | ASKYFTSDYMKKAFTFQTMYIGMSP | |
| | | | YDAPGAYNLLQATEFLEGIWYPKGG | |
| | | | FYQVVKALENIATKKFGAKFVYNCG | |
| | | | VKRIEVDADGVATGVTLETGEMIKA | |
| | | | DVVVCNADLVYAYNKLLPPTNYGDK | |
| | | | LGQNCQLTSSSISFYWSMKQKMDQF | |
| | | | GVHNIFLAEEYRESFDQIFNDHTLP | |
| | | | EDPSFYVNVPNRIDPTAAPPGKDSI | |
| | | | IVLVPVGHITDKEKQNFESLVQIAR | |
| | | | RKVIEIIERCLKIDNFEQYIEDETV | |
| | | | NHPLIWSEKFNLWKGSILGLSHNIP | |
| | | | QVLYFRPSTRSHLFKRLYFVGASAH | |
| | | | PGTGVPVVLCGSKLVADQIAKDRLT | |
| | | | SKSDRDSIVKMILVTMFFLLLAVLV | |
| | | | STMF | |
| SEQ | Eh. | Expanded | MKPTTVIGAGFGGLALAIRLQAAGI | Erwinia |
| ID | CrtI_ | Sequences | PVLLLEQRDKPGGRAYVYEDQGFTF | herbicola |
| NO: | Q47845 | | DAGPTVITDPSAIEELFTLAGKQLK | |
| 174 | | | DYVELLPVAPFYRLCWESGKVENYD | |
| | | | NDQAQLEAQIQQFNPRDVEGYRQFL | |
| | | | DYSRAVFKEGYLKLGTVPFLSFRDM | |
| | | | LRAAPQLAKLQAWRTVYSKVASYIE | |
| | | | DEHLRQAFSFHSLLVGGNPFATSSI | |
| | | | YTLIHALEREWGVWFPRGGTGALVK | |
| | | | GMIKLFQDLGGEVVLNAKVSHMETT | |
| | | | GDTIEAVHLEDGRRFPTRAVASNAD | |
| | | | VVHTYRDLLSQHPAAVKQSKKLQTK | |
| | | | RMSNSLFVLYFGLNHHHDQLAHHTV | |
| | | | CFGPRYRELIHEIFNHDGLADDESL | |
| | | | YLHAPCVTDSSLAPEGCGSYYVLAP | |
| | | | VPHLGTANLDWTVEGPRLRDRIFEY | |
| | | | LEQHYMPGLRSQLVTQRMFTPFDER | |
| | | | DQLNAYQGSAFSVEPVLTQSAWFRP | |
| | | | HNRDKTINNLYLVGAGTHPGAGIPG | |
| | | | VIGSAKATAGLMLEDLI | |
| SEQ | El. | Expanded | MNADQNIATGLNFAPANTGERGINP | Erythrobacter |
| ID | CrtI_ | Sequences | VIAEKYKGRTACVIGSGFGGLALAL | longus OCH101 |
| NO: | O06757 | | RLQSHGIQTTIVEARDKPGGRAYFW | |
| 175 | | | EKDGFTFDAGPTVITDPPCLKELWE | |
| | | | LTGHDISEDVELMKVHPFYRLNWPD | |
| | | | GTNFDYSNVDEELNAEIAKLNPDDV | |
| | | | IGYQKFLEYSARVHEEGYVKLGTVP | |
| | | | FLDFKSMLKAAPALVKERAWRSVYD | |
| | | | MVSSYIKDERLREAFSFHTLLVGGS | |
| | | | PMKTSAIYALIHKLEKDGGVWWARG | |
| | | | GTNRLIAGMVRHFERLGGTMRIGDP | |
| | | | VVQVHTQGTKATEVETKSGWKERED | |
| | | | AVCSNADIMHSYKELLGESDRGRKY | |
| | | | AKSLARKSYSPSLFVVHFGLEGSWP | |
| | | | GIAHHMILFGPRYKELVDDIYKHGV | |
| | | | LPQDFSIYLHHPTVTDPSMAPKGMS | |
| | | | TFYALVPVAHLGKMPIDWDVEGPKF | |
| | | | EKAILDEIGRRLIPDIHDRIVTKES | |
| | | | YAPKDFQADLNAHMGSAFSLETVLW | |
| | | | QSAYMRGHNRDDVIDNFYLVGAGTH | |
| | | | PGAGIPGVVGSAKATAGLMLEDLSV | |
| | | | K | |
| SEQ | Eu. | Expanded | MKPTTVIGAGFGGLALAIRLQAAGI | Erwinia |
| ID | CrtI_ | Sequences | PVLLLEQRDKPGGRAYVYEDQGFTF | uredovora |
| NO; | P21685 | | DAGPTVITDPSAIEELFALAGKQLK | |
| 176 | | | EYVELLPVTPFYRLCWESGKVENYD | |
| | | | NDQTRLEAQIQQFNPRDVEGYRQFL | |
| | | | DYSRAVFKEGYLKLGTVPFLSFRDM | |
| | | | LRAAPQLAKLQAWRSVYSKVASYIE | |
| | | | DEHLRQAFSFHSLLVGGNPFATSSI | |
| | | | YTLIHALEREWGVWFPRGGTGALVQ | |
| | | | GMIKLFQDLGGEVVLNARVSHMETT | |
| | | | GNKIEAVHLEDGRRFLTQAVASNAD | |
| | | | VVHTYRDLLSQHPAAVKQSNKLQTK | |
| | | | RMSNSLFVLYFGLNHHHDQLAHHTV | |
| | | | CFGPRYRELIDEIFNHDGLAEDESL | |
| | | | YLHAPCVTDSSLAPEGCGSYYVLAP | |
| | | | VPHLGTANLDWTVEGPKLRDRIFAY | |
| | | | LEQHYMPGLRSQLVTHRMFTPFDER | |
| | | | DQLNAYHGSAFSVEPVLTQSAWFRP | |
| | | | HNRDKTITNLYLVGAGTHPGAGIPG | |
| | | | VIGSAKATAGLMLEDLI | |
| SEQ | Eu. | Expanded | MNAHSPAAKTAIVIGAGFGGLALAI | Erwinia |
| ID | CrtI_ | Sequences | RLQSAGIATTLVEARDKPGGRAYVW | uredovora |
| NO: | P54978 | | HDQGHLFDAGPTVITDPDALKELWA | |
| 177 | | | LTGQDMARDVTLMPVSPFYRLMWPG | |
| | | | GKVFDYVNEADQLERQIAQFNPDDL | |
| | | | EGYRRFRDYAEEVYQEGYVKLGTVP | |
| | | | FLKLGQMLKAAPALMKLEAYKSVHA | |
| | | | KVATFIKDPYLRQAFSYHTLLVGGN | |
| | | | PESTSSIYALIHALERRGGVWFAKG | |
| | | | GTNQLVAGMVALFERLGGQMLLNAK | |
| | | | VARIDTDGPRATGVTLADGRALTAD | |
| | | | MVASNGDVMHNYRDLLGHTARGQSR | |
| | | | AKSLNAKRWSMSLFVLHFGLREAPK | |
| | | | DVAHHTILFGPRYKELVNEIFKGPK | |
| | | | LAEDFSLYLHSPCTTDPEMAPPGMS | |
| | | | THYVLAPVPHLGRADIDWAVEGPRY | |
| | | | ADRILASLEERLIPNLRANLTTTRI | |
| | | | FTPSDFASELNAHHGSAFSVEPILT | |
| | | | QSAWFRPHNRDKTIRNFYLVGAGTH | |
| | | | PGAGIPGVVGSAKATAQVMLSDLAS | |
| | | | A | |
| SEQ | Ev. | Expanded | MKKTVVIGAGFGGLALAIRLQAAGI | Escherichia |
| ID | CrtI_ | Sequences | PTVLLEQRDKPGGRAYVWHDQGFTF | vulneris |
| NO: | P22871 | | DAGPTVITDPTALEALFTLAGRRME | |
| 178 | | | DYVRLLPVKPFYRLCWESGKTLDYA | |
| | | | NDSAELEAQITQFNPRDVEGYRRFL | |
| | | | AYSQAVFQEGYLRLGSVPFLSFRDM | |
| | | | LRAGPQLLKLQAWQSVYQSVSRFIE | |
| | | | DEHLRQAFSFHSLLVGGNPFTTSSI | |
| | | | YTLIHALEREWGVWFPEGGTGALVN | |
| | | | GMVKLFTDLGGEIELNARVEELVVA | |
| | | | DNRVSQVRLADGRIFDTDAVASNAD | |
| | | | VVNTYKKLLGHHPVGQKRAAALERK | |
| | | | SMSNSLFVLYFGLNQPHSQLAHHTI | |
| | | | CFGPRYRELIDEIFTGSALADDESL | |
| | | | YLHSPCVTDPSLAPPGCASFYVLAP | |
| | | | VPHLGNAPLDWAQEGPKLRDRIFDY | |
| | | | LEERYMPGLRSQLVTQRIFTPADFH | |
| | | | DTLDAHLGSAFSIEPLLTQSAWFRP | |
| | | | HNRDSDIANLYLVGAGTHPGAGIPG | |
| | | | VVASAKATASLMIEDLQ | |
| SEQ | Fp. | Expanded | MNQMPRDLPNKTKTAVVIGAGEGGL | Fulvimarina |
| ID | CrtI_ | Sequences | ALAIRLQAAGIQTTLLEKRDKPGGR | pelagi HTCC2506 |
| NO: | Q0G4C1 | | AYVYEDQGFTFDAGPTVITDPSALE | |
| 179 | | | ELFETANAKLSDYVELLPVKPFYRL | |
| | | | AWEDGEVEDYADDQEDLDRQIGAKN | |
| | | | PKDVEGYRRFLAYSRDVFHEGYEKL | |
| | | | GTVPFLNFKDMMRAAPQLVRLEAYR | |
| | | | SVYSKVAQFIEDDQLRQAFSFHSLL | |
| | | | VGGNPFATSSIYALIHALERKWGVE | |
| | | | FPRGGTGALVRGMAKLFTDIGGRIE | |
| | | | VNAEVENIAIENGRAKSVTTKGGQT | |
| | | | FPADFVASNADVVHTYAKLMGRSER | |
| | | | GKKHGNSLKKKRFSMSLFVIYFGLK | |
| | | | THRPDIAHHTVCFGPRYRPLIDEIF | |
| | | | KGKELAGDFSLYLHNPCVTDPSLAP | |
| | | | EGMGSFYVLSPVPHLGNADIDWAVE | |
| | | | GPKYRDRILDYLEELYIPGLKDDLV | |
| | | | TSRIFTPADFKTELNAHLGSAFSLD | |
| | | | PVLTQSAWFRPHNRDDQIPNLYVVG | |
| | | | AGTHPGAGVPGVVGSAKATAGLMIE | |
| | | | DAGLACVPA | |
| SEQ | Gf. | Expanded | MSDIKKSVIVIGAGVGGVSTAARLA | Gibberella |
| ID | CrtI_ | Sequences | KAGFKVTILEKNDFTGGRCSLIHND | fujikuroi |
| NO: | Q8X0Z0 | | GHRFDQGPSLLLLPRFFHEIFQDLG | |
| 180 | | | TSLTAEGVELLKCEPNYNIWFGDGS | |
| | | | SFEMSTDLTKMKKAIEAVEGIDGFE | |
| | | | RYLGFLQESHRHYEVSVESVLRRNF | |
| | | | PSILSLARPEVLENLFNIHPLESIW | |
| | | | TRASKYFWTERLRRVFTFGSMYMGM | |
| | | | SPFDAPGTYSLLQYTELAEGILYPR | |
| | | | GGFHKVVEALVNVGQRLGVEYRLST | |
| | | | GVKSISIDQATGKANGVVLSDGTHL | |
| | | | PSDIVISNADLVYTYNNLLPKTSYA | |
| | | | DSLSKRETSCSSISFYWSASKIVPE | |
| | | | LNAHNIFLADEYQESFDSIFKEHLI | |
| | | | PSEPSFYVNVPSRIDPSAAPEGKDS | |
| | | | IVVLVPVGHLLSDSEGTHRGLSKSG | |
| | | | NSGGLETSQDWDKMISLARDTVIAT | |
| | | | MRARIGVDLAPLIENEIINTPFTWQ | |
| | | | EKFNLDKGAILGLSHSIMNVLAFRP | |
| | | | GTQHSKYKNLYFAGASTHPGTGVPV | |
| | | | CIAGSKIVAEQILKDSGFKNNQIPW | |
| | | | AQDTTKSPKGGLDKMSDSSLTLFQG | |
| | | | FLGALVAILLAYYYLVIAAN | |
| SEQ | Gv. | Expanded | MLRSVIKRRTRKGAALERKKAIVIG | Gloeobacter |
| ID | CrtI_ | Sequences | SGVGGLSLGIRLQSLGEDTTIFEKL | violaceus |
| NO: | Q7NM99 | | DGPGGRAYVRRAEGFTFDMGPTVIT | |
| 181 | | | VPHFIEELFALERDQPALDKPDFPA | |
| | | | AVLDENRRITSGVSGGPATSKYVQI | |
| | | | IPILPFYRIYFDDGTHEDYDGDPER | |
| | | | TREQIRAIAPEDLEGYERFHRDARA | |
| | | | IFERGFLELGFTYFRDVPSMLRIAP | |
| | | | DLVKLDAVRPLFSFVKRYFKSDKLR | |
| | | | QVFSFEPLLVGGNPLAVPAIYAMIH | |
| | | | FVEKTWGIHFAMGGTGALVRGFVRK | |
| | | | FEELGGTMRYGSEVARIEIEGSGKH | |
| | | | RRATAVVPADGSRHPADLVASNGDY | |
| | | | VNTYLKLIDRVHRPSHPDIRLKLAR | |
| | | | QSMSLLVVYFGFRSDGLNLDLRHHN | |
| | | | IILGPRYEELLKDIFGRKILAEDES | |
| | | | QYLHIPSITDPSLAPPGHHCAYTLV | |
| | | | PVPHNGSGLDWSKLADPFVDRVLRF | |
| | | | LDERGYIPGLGERLVYKSFITPDYF | |
| | | | EHTLNSRLGNGFGIEPVLWQSAYER | |
| | | | PHNKSEDIANFYLVGANTQPGGGTP | |
| | | | AVMMSAKMTAREIARDFDIPFDVVD | |
| | | | RPQVRGTVAK | |
| SEQ | Hv. | Expanded | MAQKHVIVIGAGAGGVATAARLARQ | Hesseltinella |
| ID | CrtI_ | Sequences | GFKVTVVEKNDINGGRCSLIHHDGF | vesiculosa |
| NO: | A0A1X2GT92 | | RFDQGPSLYLMPKLFEDCFADLDEN | |
| 182 | | | VQDHLDLLRCGNNYKVHFDDGDKIH | |
| | | | LTSDLTKMKDEMDRVEGDQGFANFL | |
| | | | SFMSESHVHYQQGTLLAIKRNFESV | |
| | | | WDMIKLKYAPEIFRLHLLDNVYARA | |
| | | | SRYFKTKKMRMAFTFQTMYMGMSPY | |
| | | | DAPAVYSLLQYSEFAEGIWYPRGGF | |
| | | | NMVVQKLEAIARDKFGAKFLYESPV | |
| | | | SKINTEGRQVTGVTLKDGRVLEADA | |
| | | | VVCNADLVYAYHHLLPPCHWTTRTL | |
| | | | GSKKLTSSSISFYWSMSRKIDQLDV | |
| | | | HNIFLAENYRESFDEIFQDLNLPSE | |
| | | | ASFYVNVPSRIDPSAAPGDKDAVIV | |
| | | | LVPIGHMQHVEGKWSEGFYDKMVAR | |
| | | | ARAMVLQVLERRLGMTDFGALIQNE | |
| | | | IVNHPGVWQQKENLWRGSILGLSHD | |
| | | | ILQVLWLRPSTKDSTGREDNLFFVG | |
| | | | ASTHPGTGVPIVLAGSKLTSDQVCK | |
| | | | HFHQQPLPRKKQDAEKALLNYAYLG | |
| | | | EDQASFAFGWTWLLAMFIMTLAFFL | |
| | | | AFPKDDVAKTTAASYINARLPEVFR | |
| | | | VMGNNCSGDVGQAC | |
| SEQ | Jf. | Expanded | MVDPSQKHIIIIGAGVGGTATAARL | Jimgerdemannia |
| ID | CrtI_ | Sequences | GKQGFRVTVLEKNSFSGGRCSLIHH | flammicorona |
| NO: | A0A433D8F1 | | NGHRFDQGPSLYLMPKMFEETFDEL | |
| 183 | | | GEKLTDHIELLKCQTNYKLHFQDGE | |
| | | | SILLSPDLAHMKTEIERFEGPGEAP | |
| | | | FLKYLGFLAESHVHYEHSMTLALKR | |
| | | | NFEAWYEMFQPKFLPELFKLHLFSK | |
| | | | IYSRAEKYFSSAKMRMAFTFQTMYM | |
| | | | GMSPFDAPGTYNLLQYTELAEGIWY | |
| | | | PKGGFHKVVSSLETIANKKFGAQFR | |
| | | | YDAPVKQINTAADGKTVTGVTLESG | |
| | | | EVIHADAVVCNADLVYAYNKLLPPS | |
| | | | NYGKRIGKASFTSSSISLYWGMKRK | |
| | | | IPELDVHNIFLADAYKESFDEIFKD | |
| | | | HTLPSEPSFYINVPSRIDPTAAPEN | |
| | | | KDTMVVLVPVGHMTDAHPQDYDSLV | |
| | | | AKARSLVLGSLLKHTGVDFAPLIDE | |
| | | | EIFNDPRTWQAKENLWQGSILGLSH | |
| | | | NILQVLWFRPSTKDGSGRYDNLFFV | |
| | | | GASTHPGTGVPIVLCGARLTSDQVG | |
| | | | RHFGVSDEKRGVVKAKGKKVDGAGM | |
| | | | IGWFLVLLAVLAVVVGMTGTNGLNN | |
| | | | VWEGALKCINTSLLSIIRFQL | |
| SEQ | Lc. | Expanded | MSSTQKHIIVIGAGAGGTATAARLA | Lichtheimia |
| ID | CrtI_ | Sequences | REGYRVTVVEKNDENGGRCSLIHHN | corymbifera |
| NO: | A0A068RLK8 | | GHRFDQGPSLYLMPELFKECFRDLD | JMRC:FSU:9682 |
| 184 | | | ERIEDHLELLRCQNNYKVHFDDGDT | |
| | | | IQLSSDLTRMRPEMDRFEGEDGFGR | |
| | | | FLQFLKESQVHYDRGTFVAIKRNFA | |
| | | | SIWDMIKIQYLPDIFRLHLEDMIYS | |
| | | | RASKYFKTKKMRMAFTFQTMYMGMS | |
| | | | PYDAPAVYSLLQFTEFAEGIWYPRG | |
| | | | GFNMVVQKLEMIAKDKGAEFKYSAP | |
| | | | VAKINTTERSVTGVTLENGEVIDAD | |
| | | | AVVCNADLVYAYNRLLPPCSWAQNT | |
| | | | LANKKLTSSSISFYWSMKRTIPELD | |
| | | | VHNIFLAEAYRESFDEIFKDYTLPS | |
| | | | EPSFYVNVPSRIDPSAAPADKDSII | |
| | | | VLVPIGYMRSTEPDQDYPLLVKRAR | |
| | | | DMVLEVLQRRIKVDMEPLIEHEIVN | |
| | | | DPSVWQEKFNLWRGSILGLSHDVSQ | |
| | | | VLWFRPSTKDSTGRFDNLFFVGAST | |
| | | | HPGTGVPIVLAGSKLTATQVCEHFG | |
| | | | RKGLKPSKLVEHKRVYEPESGSTLM | |
| | | | SNLVTFIISLFVLLFAFFVSFPTSS | |
| | | | VYPTPAAFINACLPSTFRVVMAADI | |
| | | | F | |
| SEQ | Ma. | Expanded | MRTVGGRTDNVVVVGAGLSGLSAAL | Mycobacterium |
| ID | CrtI_ | Sequences | HLAGRGRRVTVVERGSHPGGRVGRA | aurum A+ |
| NO: | Q9K566 | | DLSGYRIDTGPTVLTMPDILDETFA | |
| 185 | | | AVGESTADRLELIRVDPAYRASFAD | |
| | | | GSSLHVHSDAATMAAEIERFAGRAE | |
| | | | ADGYLRLRSWLARLYQLEFDGFIAS | |
| | | | NFDSPLSLLTPQLAKLAAIGGERRW | |
| | | | EPMVRRFITDERLLRVFTFQALYAG | |
| | | | VPPNRALAAYAVIAYMDTVSGVYFP | |
| | | | RGGMRALPDAMAAAAADAGVEFRYD | |
| | | | ATVTELERTGSRVTAVRTDTGERFP | |
| | | | ADAVVLTTELPDTYRMLGRTPRRPL | |
| | | | RLRPAPSAVVAHIGCRATDPAADAA | |
| | | | HHTILFGGEWDRTFEQIIDEGRTMS | |
| | | | DPSLLVTRPTAGDPTLAPDGRDLLY | |
| | | | VLAPAPNTAVSDKDWDADRDAYTAQ | |
| | | | MLDTVTTRLPHLGADAEVLHVVTPQ | |
| | | | DWARQGMLAGTPFALAHTFGQTGPF | |
| | | | RPGNRVRGIDNAILAGSSTVPGVGI | |
| | | | PTAIVSGRLAADRISSNSMMGART | |
| SEQ | Ma. | Expanded | MESYDVILIGAGHNGLVCAAYLLKA | Microcystis |
| ID | CrtI_ | Sequences | GYRVLLLEQRSVPGGAATTEAVIPD | aeruginosa |
| NO: | S3IUA6 | | LAPDFKENLCAIDHEFIFLGPVIEE | SPC777 |
| 186 | | | LELKRHGLEYLFFDPTVFCPHPSGQ | |
| | | | YFLSYRSLEKTHAAIAQFCPRDADR | |
| | | | YLQFIDYWGQLMNAIGPWFNAPPQD | |
| | | | LLRIARNYNSSALASVWKAIGSKKK | |
| | | | LLDFIRTMITSPEDVLNEWFESEFV | |
| | | | KAPLARLAAEIGAPPSQKGITSGMM | |
| | | | MMAMRHSPGVARPRGGTGALTEALV | |
| | | | KCVVSLGGVILTEQKVKQILIEEGQ | |
| | | | AIGVRVDSGGEYLAKAGVISNIDAR | |
| | | | RVFLQLVAPADVDAADPELRERVER | |
| | | | RIVNNNETILKIDCALAEAPRFEAY | |
| | | | DHKEEYLQGTILIADSVRHVEQAHA | |
| | | | LTILGQIPDENPSMYLDVPTILDPS | |
| | | | MAPQGKHTLWIEFFAPYQIAGAEGT | |
| | | | GLNGTGWTEELKNRVADRVLDKLAD | |
| | | | YAPNLKSSIIARRVESPAELANRLG | |
| | | | SYKGNYYHLDMTLDQMIFLRPLPEI | |
| | | | ANYKTPIKNLYLTGAGTHPGGSISG | |
| | | | MPGRNCAQVFLRDQSTFLQRLEN | |
| SEQ | Mc. | Expanded | MSSQEKQKHIIVIGAGAGGTACAAR | Mucor |
| ID | CrtI_ | Sequences | LAREGFKVTVVEKNDENGGRCSLIH | circinatus |
| NO: | A0A8H7S8T5 | | HEGYRFDQGPSLYLMPKLFKETFAD | |
| 187 | | | LDENINAHLNLLRCQNNYKIHFEDG | |
| | | | DKIHLTSDLTKMKPEMERVEGTHGF | |
| | | | SRFLDELNETHEHYERGTLIAIKRN | |
| | | | FESLWDMIRIQYAPEIFKLHLFDRI | |
| | | | YARASKYFKTKKMRMAFTFQTMYMG | |
| | | | MSPYDAPAVYSLLQYTEFAEGIWYP | |
| | | | EGGFNMVIQKLEKIARDKYGATFRY | |
| | | | ESPVAKINTQGNQATGVTLESGEVI | |
| | | | EADAVVCNADLVYAYHKLLPPCHWT | |
| | | | QNTLASKKLTSSSISFYWSMDRKVT | |
| | | | ELDVHNIFLAEAYQESFDEIFVDNT | |
| | | | LPSEPSFYVNVPSRIDPSAAPADKD | |
| | | | SIIVLVPIGHMRGADAASTPEDYQD | |
| | | | LVNRAREMVLSVLCRRLGLAHFKDW | |
| | | | IAHEIVNDPAVWQQKFNLWRGSILG | |
| | | | LSHDVLQVLWFRPSTKDSTGREDNL | |
| | | | FFVGASTHPGTGVPIVLAGSKLTSN | |
| | | | QVCNHFSKTPKPSQLLLKEEEQKII | |
| | | | YKPESGMTPSYWYSVLSLFILVFAF | |
| | | | FVSFPSEQFGHPTAAAYINSCLPAQ | |
| | | | FRVAVYE | |
| SEQ | Mc. | Expanded | MSKKHIVIIGAGVGGTATAARLARE | Mucor |
| ID | CrtI_ | Sequences | GFKVTVVEKNDFGGGRCSLIHHEGH | circinelloides |
| NO: | S2J0C7 | | RFDQGPSLYLMPKYFEDAFADLDER | f. |
| 188 | | | IQDHLELLRCDNNYKVHEDDGESIQ | circinelloides |
| | | | LSSDLTRMKAELDRVEGPLGFGREL | |
| | | | DEMKETHIHYESGTLIALKKNFESI | |
| | | | WDLIRIKYAPEIFRLHLFGKIYDRA | |
| | | | SKYFKTKKMRMAFTFQTMYMGMSPY | |
| | | | DAPAVYSLLQYTEFAEGIWYPRGGF | |
| | | | NMVVQKLEMIAKTKYGADFIYNAPV | |
| | | | AKINTNDTTKQVTGVTLENGQIIDA | |
| | | | DAVVCNADLVYAYHNLLPPCRWTQN | |
| | | | TLASKKLTSSSISFYWSMSTKVPQL | |
| | | | DVHNIFLAEAYQESFDEIFKDFGLP | |
| | | | SEASFYVNVPSRIDPSAAPDGKDSI | |
| | | | IVLVPIGHMKSKTGDATTENYPAMV | |
| | | | NRARKMVLEVIERRLDMSNFADLIE | |
| | | | HEQVNDPAVWQSKENLWRGSILGLS | |
| | | | HDVLQVLWFRPSTKDSTGRYDNLFF | |
| | | | VGASTHPGTGVPIVLAGSKLTSDQV | |
| | | | VRSFGKSPKPRKIEIENKQAPLEQD | |
| | | | VAIGFSLGMWLRIAFLVVFMFFYFF | |
| | | | PQSNGQTPASFINNLLPDVFRVHNS | |
| | | | NVI | |
| SEQ | Mi. | Expanded | MASQKHIVIIGAGVGGTATAARLAK | Mortierella |
| ID | CrtI_ | Sequences | QGFRVTVVEKNSFSGGRCSLIYKNG | isabellina |
| NO: | A0A8H7Q0C1 | | HRFDQGPSLYLMPKIFKEAFADLDE | |
| 189 | | | DIEDHIDLLKCENNYKVHFHDGDSI | |
| | | | HLTSDLVKMKTEIERYEGEGEKPFL | |
| | | | NYLRFLSESHIHYERSLVMALKRNF | |
| | | | EAWYEMFQPKYAPEVENLHLFHKVY | |
| | | | TRAAKYFSSKKMRMAFTFQSMYMGM | |
| | | | SPFDAPATYNLLQYTEFAEGIWYPR | |
| | | | GGFNMVVQRLETIATAKYGAQFIYN | |
| | | | APVAKINVDEKAKLVTGITLENGQV | |
| | | | VQADAVVCNADLIYAYNNLLPPCSW | |
| | | | SKRIGEAKLTSSSISFYWCMKRKIP | |
| | | | ELDVHNIFLAQAYQKSFDQIFKEHT | |
| | | | LPSEPSFYVNVPSRIDPSAAPEGKD | |
| | | | SVIVLLPIGHMLPGRPEDYQKLVAK | |
| | | | ARTMILTTLEKRLGFDISKEIEHEE | |
| | | | YNDPTVWQEKFNLWRGSILGLSHDI | |
| | | | LQVLWFRPSTKDSTGRENNLFFVGA | |
| | | | STHPGTGVPIVLCGSKLTSDQVTKH | |
| | | | FGYDIKNQKKRTIAYAQNDGTTSIW | |
| | | | YGAIIMFMLVFAFFYGFPKDKGTAA | |
| | | | SFINSYLPLSMRAY | |
| SEQ | Mp. | Expanded | MSKKHIVIIGAGVGGTATAARLARE | Mucor plumbeus |
| ID | CrtI_ | Sequences | GFKVTVVEKNDFGGGRCSLIHHNGH | |
| NO: | A0A8H7RIY5 | | RFDQGPSLYLMPKYFEDAFADLDER | |
| 190 | | | IEDHLELLRCDNNYKVHEDDGEAIQ | |
| | | | LSSDLTRMKSELDRVEGPLGFGRFL | |
| | | | DEMKETHIHYESGTLIALKKNFESI | |
| | | | WDLIRIKYAPEIFRLHLFGKIYGRA | |
| | | | SKYFKTKKMRMAFTFQTMYMGMSPY | |
| | | | DAPAVYSLLQYTEFAEGIWYPRGGF | |
| | | | NMVVQKLEAIAKNKYDAEFIYNAPV | |
| | | | SKINTNDTTKQVTGVTLENGEIIEA | |
| | | | DAVICNADLVYAYHNLLPPCRWTQN | |
| | | | TLASKKLTSSSISFYWSMSTKVPQL | |
| | | | DVHNIFLAEAYQESFDEIFKDFGLP | |
| | | | SEASFYVNVPSRIDPTAAPADKDSI | |
| | | | IVLVPIGHMKSKTGDATIENYPEMV | |
| | | | KKARKMVLEVIERRLGMSNFSNLIE | |
| | | | HEQVNDPAIWQSKENLWRGSILGLS | |
| | | | HDVFQVLWFRPSTKDSTGRYNNLFF | |
| | | | VGASTHPGTGVPIVLAGSKLTSDQV | |
| | | | VKSFGKAPRARKIEIENKQAPLEEN | |
| | | | GQISSSFSQLWVWLRVIFWVLFLFF | |
| | | | YFFPQSNNGQTPASFINNHLPNVFR | |
| | | | AKDSYLDVDMI | |
| SEQ | Ms. | Expanded | MSDNKKHIVVIGAGIGGVATAARLS | Mucor |
| ID | CrtI_ | Sequences | REGFRVTVVEKNDFSGGRCSLIYNE | saturninus |
| NO: | A0A8H7RGU3 | | GHRFDQGPSLYLMPKLFQDAFADLD | |
| 191 | | | ERIEDHVDLLRCDNNYKVHFDDGES | |
| | | | IQLSSDLTRMKSELDRIEGPEGFGR | |
| | | | FLDELKETHIHYERGVFIAIKRNFE | |
| | | | SIWDMVRIKFAPEIFRLHLFGKIYD | |
| | | | RASKYFLTKKMRMAFTFQTMYMGMS | |
| | | | PYDAPAVYSLLQYTEFAEGIWYPRG | |
| | | | GFNVVVQKLEEIGSKKYGAKFIYNA | |
| | | | PVAKINTEDENKRVTGVTLENGEVI | |
| | | | HADAVVCNADLVYAYHHLLPPCSWT | |
| | | | TNTLATKKLTSSSISFYWSLKEKVP | |
| | | | LLDVHNIFLAEAYQESFDEIFKDYG | |
| | | | LPSEASFYVNVPSRIDPSAAPEGKD | |
| | | | SVIVLVPIGHMRSLTGNGDEENYPK | |
| | | | MVERARAMVLEIIQRRLGMENFADL | |
| | | | IEHESHNDPAIWQSKENLWRGSILG | |
| | | | LSHDILQVLWFRPSTKDSTLRYDNL | |
| | | | FFVGASTHPGTGVPIVLAGSKLTSD | |
| | | | QVTKSFGKAPLPRKIQDEPTYKAEA | |
| | | | NDASSQLVTYSVYLFIITFLFFVLF | |
| | | | PTQSTNHTAASFINSYLPKQFRVTY | |
| | | | DNDLLLERY | |
| SEQ | Mx. | Expanded | MASEGGSVRHVIVVGAGPGGLSAAI | Myxococcus |
| ID | CrtI_ | Sequences | NLAGQGFRVTVVEKDAVPGGRMKGL | xanthus |
| NO: | Q02861 | | TLGASGEYAVDTGPSILQLPGVLEQ | |
| 192 | | | IFRRAARRLEDYVKLLPLDVNTRVH | |
| | | | FWDGTHLDTTRHLDRMEAELAKFGP | |
| | | | RQASALRQWMEDGREKYGIAYQKFI | |
| | | | CTSADNLGYYAPWRLAPTLRFKPWQ | |
| | | | TLYRQLDGFFHDDRVTYALAYPSKY | |
| | | | LGLHPTTCSSVFSVIPFLELAFGVW | |
| | | | HVEGGFRELSRGMMRCARDLGATER | |
| | | | MGTPVEKVRVDAGRAVGVKLVGGEV | |
| | | | LDADAVVVNADLAYAARSLIPAEAR | |
| | | | EGSRLTDAALERAKYSCSTFMAYYG | |
| | | | LDTVYADLPHHLIYLSESARRTDRD | |
| | | | ALEDRHVDLEDPPFYVCNPGVTDPS | |
| | | | GAPAGHSTLYVLVPTPNTGRPVDWV | |
| | | | KTEQALRERIPAMLEKVGLKGVREH | |
| | | | IREERYFTAETWRDDENVERGAVEN | |
| | | | LSHTWLQLGPLRPKVKNRDIEGLYF | |
| | | | VGGGTHPGSGLLTIMESANIAADYL | |
| | | | TREAGKGPLPGWPYVPPLEPESPVQ | |
| | | | ARAG | |
| SEQ | Mx. | Expanded | MSASTQGRRIVVVGAGVGGLAAAAR | Myxococcus |
| ID | CrtI_ | Sequences | LAHQGFDVQVFEKTQGPGGRCNRLQ | xanthus DK |
| NO: | Q1DDW7 | | VDGFTWDLGPTIVLMPEVFEETFRA | 1622d |
| 193 | | | VGRRIEDYLTLLRCDPNYRVHFRDG | |
| | | | SDVTFTSELCAMGRELERVEPGSYA | |
| | | | RYLAFLAQGRVQYRTSLDHLVGRNY | |
| | | | AGLRDYLSPRVLARIFQVRAHRRMY | |
| | | | ADVSRFFQDERLRAAMTFQTMYLGV | |
| | | | SPYASPAVYGLLPFTELGVGIWFPK | |
| | | | GGLYAIPQALERLAREEGVRFHYGA | |
| | | | PVERILTDGGRTRGVRLEGGEVVEA | |
| | | | DAVLCNADLPYAYEKLLDPKATTLK | |
| | | | RKEKLRYTSSGYMLYLGMKRRYPEL | |
| | | | LHHNVVFGRDYKGSFDDIFERFRVP | |
| | | | EDPSFYVNAPTRTDASLAPEGKDAL | |
| | | | YVLVPVPHQHPDLDWKVEGPKVRAK | |
| | | | FFARMAELGFPSLESDIEVERVFTP | |
| | | | DDWAGTENLARGSAFGLSQNFTQIG | |
| | | | PFRPSNQDARVKNLFFVGASTQPGT | |
| | | | GLPTVLISARLVTERLMTWAHAQGV | |
| | | | SLSPRTAAATPLEGVAA | |
| SEQ | Pb. | Expanded | MTGQKEAGQKEACVIGGGFGGLALA | Parvularcula |
| ID | CrtI_ | Sequences | CRLQAAGLATTLIEGRDKLGGRAYV | bermudensis |
| NO: | E0THV8 | | YSLEGYTFDAGPTVVTDPSALEEIF | HTCC2503 |
| 194 | | | AMAGRRLSDYVELLNVDPLYRLHWE | |
| | | | DGYSFDYAKDEEHLLSQIRAKNPKD | |
| | | | VEGYRKFYRYSEAVFKEGYEKLGAV | |
| | | | PFLNFWSMVKAAPQLTTLQAFRSVY | |
| | | | GRVADFIEDEQLRQAFSFHTLLVGG | |
| | | | DPHKTSSIYALIHALERKWGVWFPR | |
| | | | GGTGALIEGLGRLFTDLGGTLRLED | |
| | | | PVTDLKVQNDRITQVTTESGWSHQF | |
| | | | DAVASNADIVHTYDNLLGGSKRGPQ | |
| | | | AAKGLKQKRFSNSLFVTYFGTKKQY | |
| | | | PDIAHHSILFGPRYRELIAEIFSGP | |
| | | | QLPNDFSLYLHRPTATDPGLAPPGC | |
| | | | DAFYVLSPVPNLEKAGIDWSVTGPE | |
| | | | YQERILAYLEERYLPGLRDNLAVTH | |
| | | | RFTPADFKDKLNAHVGSAFSLEPVL | |
| | | | TQSAYFRLHNRDDKIGNLYFVGAGT | |
| | | | HPGAGIPGVVGSAKATAGLMIEDMG | |
| | | | LSRSNRGPKAA | |
| SEQ | Pb. | Expanded | MAPPKHVIIIGAGAGGTATAARLAR | Phycomyces |
| ID | CrtI_ | Sequences | EGIKVTVVEKNNFGGGRCSLINHNG | blakesleeanus |
| NO: | P54982 | | HRFDQGPSLYLMPKLFEEAFEALDE | |
| 195 | | | KIEDHVELLRCHNNYKVHFDDGDKI | |
| | | | QLSSDLSRMKPEMERIEGPDGFLRF | |
| | | | LDEMKESHTHYEGGVEMAIKQNFET | |
| | | | IWKLIRLQYVPALFRLHIFDFVYSR | |
| | | | AAKYFKTKKMRMAFTFQSMYMGMSP | |
| | | | YDSPAVYNLLQYTEFAEGIWYPKGG | |
| | | | FNTVIQKLENIATEKFGARFIYEAP | |
| | | | VAKINTDDKGKKVTGVTLQSGEVIE | |
| | | | ADAVVCNADLVYAYHNLLPPCRWTT | |
| | | | NTLAEKKLTSSSISFYWSLKRVVPE | |
| | | | LDVHNIFLAEAFKESFDEIFTDHKM | |
| | | | PSELSFYVNLPSRIDPTAAPPGKDS | |
| | | | MIVLVPIGHMKSKTNEAEDYTMIVK | |
| | | | RARKMVLEVLERRLGLTNFIDLVEH | |
| | | | EEVNDPSIWQKKENLWRGSILGLSH | |
| | | | DVLQVLWFRPSTQDSTGRYKNLFFV | |
| | | | GASTHPGTGVPIVLAGSKLTSDQVC | |
| | | | DHFGVKVRPSAITSSKRTYAPEDSK | |
| | | | SFIWDIIWELLIALFAATLVLFIAF | |
| | | | PQYSEVNQTAASYINNLLPAAFRVP | |
| | | | VANLSLTS | |
| SEQ | Pf. | Expanded | MTGPKRTAKVAIIGAGPGGLAAAML | Paenibacillus |
| ID | CrtI_ | Sequences | LAAEGYAVDLYEKQDTVGGRSGELK | sp. FSL P4-0081 |
| NO: | A0A089KIW6 | | LGSYRFDRGATFLMMPHLLEELFAL | |
| 196 | | | AGRSVHDYVSLKPLDPLYSLHEGDT | |
| | | | VFTPSTDQEQTAGEIERLFPGNGSG | |
| | | | YRRFMADEADKLERVIPLLQRPFQS | |
| | | | LGDYMKRDVLHALPKLNAADNVYNR | |
| | | | LSRYFDDERLRFSFTFQAKYLGMSP | |
| | | | WECPGTFTILSYMEHRYGLYHPIGG | |
| | | | INRVLQAMSEVIQEHGGHVHTSSGV | |
| | | | KRIVVKNGDAAGLLLENGERVEADY | |
| | | | VVIGADFGSAMTQLFEPGILKKYTP | |
| | | | RKVARKRYSCSTAMLYLGIDGEVEL | |
| | | | GHHSVHFSADYRRNVEEITKLGVLS | |
| | | | EDPSIYVHNPSVIDKTLAPPGKSSL | |
| | | | YVLMPVPNLSAEVNWQQTSAEVEAR | |
| | | | MMERLEQIPQLAGITGRVEESLFFS | |
| | | | PLDWQNKLNVYNGATENLAHNLGQM | |
| | | | MHLRPHNTFEEVGGIWLVGGGTHPG | |
| | | | SGLPTIFESAKISARLLREHDQALR | |
| | | | GSFTVPAGAPGAGAGVPL | |
| SEQ | Ph. | Expanded | MNAHSPAAKTAIVIGAGFGGLALAI | Paracoccus |
| ID | CrtI_ | Sequences | RLQSAGIATTLVEARDKPGGRAYVW | haeundaensis |
| NO: | Q24K61 | | HDQGHVFDAGPTVITDPDALKELWA | |
| 197 | | | LTGQDMARDVTLMPVSPFYRLMWPG | |
| | | | GKVFDYVNEADQLERQIAQFNPDDL | |
| | | | EGYRRFRDYAEEVYQEGYVKLGTVP | |
| | | | FLKLGQMLKAAPALMKLEAYKSVHA | |
| | | | KVATFIKDPYLRQAFSYHTLLVGGN | |
| | | | PESTSSIYALIHALERRGGVWFAKG | |
| | | | GTNQLVAGMVALFERLGGQMMLNAK | |
| | | | VARIETEGARTTGVTLADGRSLRAD | |
| | | | MVASNGDVMHNYRDLLGHTARGQSR | |
| | | | AKSLDRKRWSMSLFVLHFGLREAPK | |
| | | | DIAHHTILFGPRYRELVNEIFKGPK | |
| | | | LAEDFSLYLHSPCTTDPDMAPPGMS | |
| | | | THYVLAPVPHLGRAEIDWAVEGPRY | |
| | | | ADRILASLEERLIPNLRANLTTTRI | |
| | | | FTPADFASELNAHHGSAFSVEPILT | |
| | | | QSAWFRPHNRDKTIRNFYLVGAGTH | |
| | | | PGAGIPGVVGSAKATAQVMLSDLAG | |
| | | | A | |
| SEQ | Pp. | Expanded | MSRKHIIIIGAGVGGTATAARLARE | Parasitella |
| ID | CrtI_ | Sequences | GFKVTVVEKNNENGGRCSLIYHNGH | parasitica |
| NO: | A0A0B7NEC9 | | RFDQGPSLYLMPKYFEDAFADLDER | |
| 198 | | | IEDHLELLRCDNNYKVHFDNGESIQ | |
| | | | LSSDLTRMKAELDRVEGPLGFGRFL | |
| | | | DFLKETHIHYEKGTMIALKRNFESI | |
| | | | WDLIRIKYAPEIFRLHLEDKIYSRA | |
| | | | SKYFKTKKMRMAFTFQTMYMGMSPY | |
| | | | DAPAVYSLLQYTEFAEGIWYPRGGF | |
| | | | NMVVQKLEAIAKKYGAEFMYDAPVS | |
| | | | EINTIDGSKHVTGVTLECGRVIEAD | |
| | | | AVVCNADLVYAYHNLLPPCKWTQNT | |
| | | | LASMKLTSSSISFYWSMSTKILQLD | |
| | | | VHNIFLAEAYKESFDEIFKDFTMPS | |
| | | | EASFYVNVPSRIDPTAAPAGKDSII | |
| | | | VLVPIGHIKSKISDASSENYSKMVD | |
| | | | RARKMVLQVIECRLGMSNFADLIEH | |
| | | | EQVNDPSVWQNKFNLWRGSILGLSH | |
| | | | DVFQVLWFRPSTKDSTGRYDNLFFV | |
| | | | GASTHPGTGVPIVLAGSKLTSDQVV | |
| | | | RSFGMTPKPRKIEIKNRKALPEHDD | |
| | | | LLFSWSSRYLAWQQLIFWGIFLFFY | |
| | | | FFPQSDNGVTPASFINSFLPDVFHG | |
| | | | DNSNIHQKIM | |
| SEQ | Pz. | Expanded | MSSAIVIGAGFGGLALAIRLQSAGI | Paracoccus |
| ID | CrtI_ | Sequences | ATTIVEARDKPGGRAYVWNDQGHVF | zeaxanthinifaciens |
| NO: | P94790 | | DAGPTVVTDPDSLRELWALSGQPME | R1534 |
| 199 | | | RDVTLLPVSPFYRLTWADGRSFEYV | |
| | | | NDDDELIRQVASENPADVDGYRRFH | |
| | | | DYAEEVYREGYLKLGTTPFLKLGQM | |
| | | | LNAAPALMRLQAYRSVHSMVARFIQ | |
| | | | DPHLRQAFSFHTLLVGGNPESTSSI | |
| | | | YALIHALERRGGVWFAKGGTNQLVA | |
| | | | GMVALFERLGGTLLLNARVTRIDTE | |
| | | | GDRATGVTLLDGRQLRADTVASNGD | |
| | | | VMHSYRDLLGHTRRGRTKAAILNRQ | |
| | | | RWSMSLFVLHFGLSKRPENLAHHSV | |
| | | | IFGPRYKGLVNEIFNGPRLPDDFSM | |
| | | | YLHSPCVTDPSLAPEGMSTHYVLAP | |
| | | | VPHLGRADVDWEAEAPGYAERIFEE | |
| | | | LERRAIPDLRKHLTVSRIFSPADES | |
| | | | TELSAHHGSAFSVEPILTQSAWFRP | |
| | | | HNRDRAIPNFYIVGAGTHPGAGIPG | |
| | | | VVGSAKATAQVMLSDLAVA | |
| SEQ | Ra. | Expanded | MSKHIVIIGAGVGGTATAARLAREG | Rhizopus |
| ID | CrtI_ | Sequences | FRVTVVEKNDESGGRCSLIHHDGYR | azygosporus |
| NO: | A0A367JJ19 | | FDQGPSLYLMPKLFEDAFADLDERV | |
| 200 | | | EDHLELLRCENNYKIHFDDGESIHL | |
| | | | TSDLTRMKIEMERVEGPEGFGRELD | |
| | | | FLRETHVHYQKGTWIALKKNFESIW | |
| | | | DLIRLKYAPEILNLHLEDKIYRRAS | |
| | | | LYFKTKRMRMAFTFQTMYMGMSPYD | |
| | | | APAVYSLLQYTEFAEGIWYPRGGEN | |
| | | | QVVQKLEFIASQKYRATFRYNAPVA | |
| | | | KINTDGGRVCGVTLENGEVIEADAV | |
| | | | VCNADLVYAYHKLLPPCSWTTKTLA | |
| | | | SKKLTSSSISFYWSMNKKISQLDVH | |
| | | | NIFLAESYKESFDEIFKQYGLPSEA | |
| | | | SFYVNVPSRVDPSAAPDGKDSVIVL | |
| | | | VPIGHMKSQTGEGEDYANLVNRARE | |
| | | | MILSVIQARLGIKDFASMIEHEIVN | |
| | | | DPKTWQAKFNLWRGSILGLSHNVFQ | |
| | | | VLWFRPSTRDTTNRYRNLFFVGAST | |
| | | | HPGTGVPIILAGSKLTSDQVCKHFG | |
| | | | KTPLPRKLEYTPQAKDYVEKSHDGL | |
| | | | SWFSIFAVVVELLSLFFAFFPEQST | |
| | | | GHTAASFINSCLPKPFQVDGTSSSF | |
| | | | WVA | |
| SEQ | Ra. | Expanded | MSKHIVIIGAGVGGTATAARLAREG | Rhizopus |
| ID | CrtI_ | Sequences | FRVTVVEKNDESGGRCSLIHHDGYR | azygosporus |
| NO: | A0A367JYF3 | | FDQGPSLYLMPKLFEDAFADLDERV | |
| 201 | | | EYHLELLRCENNYKIHEDDGESIHL | |
| | | | TSDLTRMKAEMERVEGPEGFGRFLD | |
| | | | FLRETHVHYQKGTWIALKKNFESIW | |
| | | | DLIRLEYAPEILRLHLEDKIYRRAS | |
| | | | LYFKTKRMRMAFTFQTMYMGMSPYD | |
| | | | APAVYSLLQYTEFAEGIWYPRGGEN | |
| | | | QVVQKLEFIASQKYRATFRYNAPVA | |
| | | | KINVDGGRVSGVTLENGEVIEADAV | |
| | | | VCNADLVYAYHKLLPPCSWTTKTLA | |
| | | | SKKLTSSSISFYWSMNKKISQLDVH | |
| | | | NIFLAESYKESFDEIFKQYGLPSEA | |
| | | | SFYVNVPSRVDPSAAPDGKDSVIVL | |
| | | | VPIGHMKSQTGEGEDYADLVNRARE | |
| | | | MVLDVIQARLGIKDFASMIEHEIVN | |
| | | | DPKTWQAKFNLWRGSILGLSHNVFQ | |
| | | | VLWFRPSTRDTTNRYCNLFFVGAST | |
| | | | HPGTGVPIVLAGSKLTSDQICKHFG | |
| | | | KTPLPRKLEYAPQAKDYVEKSHNGP | |
| | | | SWLSIFAVIVELLSLFFAFFPEQST | |
| | | | GHTAASFINSYLPKPFQVEGTSSSF | |
| | | | WVA | |
| SEQ | Ra. | Expanded | MPATKHVSDADHAIVIGSGLGGLSA | Rhodobacter |
| ID | CrtI_ | Sequences | AMRLGAKGWRVTVIDKLDVPGGRGS | azotoformans |
| NO: | G3FHJ1 | | SITQEGHRFDLGPTIVTVPQSLRDL | |
| 202 | | | WATCGRDFDKDVALKPVDPFYEVRW | |
| | | | PDGSDFTVRQSTEAMKAEIARLSPG | |
| | | | DVAGYETFLKDSEKRYWEGYENLGR | |
| | | | RPMHKLWDLIKVLPTFGMLRADRSV | |
| | | | YQHAARRVQDERLRMALSFHPLFIG | |
| | | | GDPFNVTSMYILVSQLEKEFGVHYA | |
| | | | IGGVAAIAQAMAQVIEGQGGAFRMN | |
| | | | TEVDEIVVENGEAKGVRLASGEVLR | |
| | | | AGLVVSNADAGHTYMRLLRNHKRKR | |
| | | | WTDAHVKSRRWSMGLFAWYFGTKGT | |
| | | | KGMWSDVGHHTIINAPRYKGLVEDI | |
| | | | FLKGKLAKDMSLYIHRPSITDPTVA | |
| | | | PEGDDTFYALSPVPHLKHANPVDWQ | |
| | | | AMGEPYRQSVLEVLEESMPGIGDRI | |
| | | | GPSLVFTPETERDRYLSPWGAGFSI | |
| | | | EPRILQSAWFRPHNISEEVKGLFLV | |
| | | | GAGTHPGAGVPGVIGSAEVMAKLAP | |
| | | | DAPRARREAEPAERLAAE | |
| SEQ | Rc. | Expanded | MSKNTEGMGRAVVIGAGLGGLAAAM | Rhodobacter |
| ID | CrtI_ | Sequences | RLGAKGYKVTVVDRLDRPGGRGSSI | capsulatus |
| NO: | P17054 | | TKGGHRFDLGPTIVTVPDRLRELWA | |
| 203 | | | DCGRDFDKDVSLVPMEPFYTIDFPD | |
| | | | GEKYTAYGDDAKVKAEVARISPGDV | |
| | | | EGFRHFMWDAKARYEFGYENLGRKP | |
| | | | MSKLWDLIKVLPTFGWLRADRSVYG | |
| | | | HAKKMVKDDHLRFALSFHPLFIGGD | |
| | | | PFHVTSMYILVSQLEKKFGVHYAIG | |
| | | | GVQAIADAMAKVITDQGGEMRLNTE | |
| | | | VDEILVSRDGKATGIRLMDGTELPA | |
| | | | QVVVSNADAGHTYKRLLRNRDRWRW | |
| | | | TDEKLDKKRWSMGLFVWYFGTKGTA | |
| | | | KMWKDVGHHTVVVGPRYKEHVQDIF | |
| | | | IKGELAEDMSLYVHRPSVTDPTAAP | |
| | | | KGDDTFYVLSPVPNLGFDNGVDWSV | |
| | | | EAEKYKAKVLKVIEERLLPGVAEKI | |
| | | | TEEVVFTPETERDRYLSPLGAGFSL | |
| | | | EPRILQSAWFRPHNASEEVDGLYLV | |
| | | | GAGTHPGAGVPSVIGSGELVAQMIP | |
| | | | DAPKPETPAAAAPKARTPRAKAAQ | |
| SEQ | Rd. | Expanded | MDERKHVVIIGAGVGGTATAARLAR | Rhizopus |
| ID | CrtI_ | Sequences | QGFRVTVIEKNGFSGGRCSTLRHDG | delemar |
| NO: | I1CU28 | | FRFDQGPSLYLMPKVFEEAFADLDE | |
| 204 | | | RIGDHVDLLRCENNYRLHFPDGDTI | |
| | | | ELSSDLCRMQQVMDRIEGPGGMDRF | |
| | | | LGFLQEAQVHHARGTPIAIERNFER | |
| | | | IWDLVRYGSEIGRLHLLDTIYRRAS | |
| | | | RYFKTKKMRMAFTFQTMYMGMSPYD | |
| | | | APAVYSLLQYTECVEGIWYPRGGFH | |
| | | | QVVHRLEQIAQEKFGASFVYNVPVA | |
| | | | KINTQGRRVTGVTLADGEVIQADAV | |
| | | | VCNADLVYAYHQLLPPCAWTTQSLA | |
| | | | AKQLTCSSISFYWSMSRTIPALDVH | |
| | | | NVFLAEAYQASFDEIFCQHRLPSQP | |
| | | | SFYVHVPSRLDPSAAPPNKDAVVVL | |
| | | | VPIGHMHKDPVDYDRLVQRARSTVL | |
| | | | DVLHRRLGLEADWIEHEIVNDPRTW | |
| | | | QAKFNLWKGSILGLSHHLFQVLWER | |
| | | | PSTKDASGRYDNLYFVGASTHPGTG | |
| | | | VPIVLAGSKLTSDQVCRGFGQVPLP | |
| | | | RKNPSRPPSLRPPLRPLWILLVLGF | |
| | | | LGFLCSLTDQHLPLSFSRRQ | |
| SEQ | Rg. | Expanded | MLMKSDAAAPRAAGKQDPQAPVALV | Rubrivivax |
| ID | CrtI_ | Sequences | VGSGFGGMAAAVRLAAKGYRVTVLE | gelatinosus S1 |
| NO: | Q840T3 | | KLDAPGGRAYVHRREGHVFDAGPTI | |
| 205 | | | VTVPYLFDELWALAGRKESDDIELK | |
| | | | SLDPFYRIRFDDGDHFDYSGDPARM | |
| | | | RAEVRRISPSDAEGFEREMREADQC | |
| | | | YELGFRTLGDKAFDTVGDLIKAAPL | |
| | | | IIRLRGWRSLHQMVSSHLKHPKLRI | |
| | | | AMSLQSLLIGGNPFSVTSMYALVNA | |
| | | | LERQWGVHWAMGGTGELIRGLVDVF | |
| | | | EGMGGTMRLKAEVKRIEVDNGVATG | |
| | | | VTLADGERLPADIVVCNGDTGYLYK | |
| | | | NLVDARWRKHWTDARIERGHYSMGL | |
| | | | FVWYFGTDRRYEDVPHHMMVLGPRY | |
| | | | RELLDDIFRKKKLAGDFSIYLHRPT | |
| | | | ATDPSMAPAGCDTFYALMPVPHLGS | |
| | | | GTDWTTQAEPYRQSVQEALERTVLP | |
| | | | GLGQHLRVSFCTTPLDFQHRLLSYK | |
| | | | GAGFGLEPLLLQSAYFRPHNRSEDV | |
| | | | KNLFMVGASTHPGAGVPGVIMSAKA | |
| | | | LESVLPDPATFRR | |
| SEQ | Rm. | Expanded | MSKHIVVIGAGIGGTATAARLAREG | Rhizopus |
| ID | CrtI_ | Sequences | FRVTVVEKNDESGGRCSLIHHGGYR | microsporus |
| NO: | A0A2G4SFW1 | | FDQGPSLYLMPKLFEDAFADLDERV | ATCC 52813 |
| 206 | | | EDHLELLRCENNYKVHFDDGESIHL | |
| | | | TSDLTRMKTEMERVEGPEGFGRFLD | |
| | | | FLRETHVHYQNGTWIALKKNFESIW | |
| | | | DLIRLEYAPEILRLHLEDKIYRRAS | |
| | | | LYFKTKRMRMAFTFQTMYMGMSPYD | |
| | | | APAVYSLLQYTEFAEGIWYPRGGEN | |
| | | | QVVQKLESIATQKYRATFRYNAPVA | |
| | | | KINTDSGRVCGVTLENGEVIEADAV | |
| | | | VCNADLVYAYHKLLPPCSWTTKTLA | |
| | | | SKKLTSSSISFYWSMNKKIPQFDVH | |
| | | | NIFLAESYKESFDEIFEQYGLPSEA | |
| | | | SFYVNVPSRVDPSAAPDGKDSVIVL | |
| | | | VPIGHMKSQTGEGEDYADLVNRARE | |
| | | | MVLSVIQSRLGIKDFASMIEHEIVN | |
| | | | DPKTWQAKFNLWRGSILGLSHNVFQ | |
| | | | VLWFRPSTRDTTNRYRNLFFVGAST | |
| | | | HPGTGVPIVLAGSKLTSDQVCKHFG | |
| | | | KTPLPRKLGYTPQPKDYVEKSHGEP | |
| | | | SWESIFAVIVELLSFFFAFFPEQST | |
| | | | GHTAASFINSYLPKPFQVEGASSSF | |
| | | | WIV | |
| SEQ | Rr. | Expanded | MKLHPAPTVPTQDRRPHAVVIGSGF | Rhodospirillum |
| ID | CrtI_ | Sequences | GGLAAAVRLGARGYRVTVLEKLGGP | rubrum ATCC |
| NO: | Q2RX47 | | GGRGGVFKQDGFTFDAGPTIVTLPS | 11170 |
| 207 | | | LFEELWTLCGRQMSDDVDLRPLTPF | |
| | | | YTIRENDGETFRAQSDPEAMRAEVA | |
| | | | RLSPGDVEGYERFIAESEDIYKVGF | |
| | | | EQMADKPFSSITDMLKVVPQMLKLG | |
| | | | ALRSVWGHVAKHVADPRLRIALSFH | |
| | | | PLFIGGNPLSVTSIYSMIAFLERRW | |
| | | | GVHFAMGGTHSLVKGLVGLIEGQGN | |
| | | | HLRYNAGVAEILVKNDRACGVRLET | |
| | | | GEEIAADIVVSNADSAATYKNLLPA | |
| | | | AHRRWWSDRRLAWSRYSMSLFIWHF | |
| | | | GTKGTYPDVAHHTILMGPRYHTLIA | |
| | | | DIFQAKTLAKDESLYLYRPTATDPS | |
| | | | MAPEGCDSFYVLSPVPHLGGGQDWA | |
| | | | LEAEPYRKAILAELERTVLPGLGAK | |
| | | | IVTSKVVTPVDFRDGLSSLNGAAFG | |
| | | | LEPAFTQSAWFRPHNKSEEIDRLYL | |
| | | | VGAGTHPGAGLPGVLSSAKILDTVV | |
| | | | PDASVLV | |
| SEQ | Rs. | Expanded | MSQQKHIVVIGAGVGGTATAARLSR | Rhizopus |
| ID | CrtI_ | Sequences | EGFKVTVVEKNDESGGRCSLIHHNG | stolonifer |
| NO: | A0A367KGJ1 | | HRFDQGPSLYLMPKLERDAFADLDE | |
| 208 | | | RIEDHLNLLRCDNNYKVHFDDGQSI | |
| | | | QLSSDLTRMKTEIERWEGPDGFGRF | |
| | | | LDEMKEMHVHYETGVWIAIKRNFES | |
| | | | IWDLIKIKYLPEVFRLHLENKIYQR | |
| | | | ASVYFKSKRMRMAFTFQTMYMGMSP | |
| | | | YDAPAVYSLLQYTEFAEGIWYPRGG | |
| | | | FNMVVQKLESIAANKYGAKFMYNAP | |
| | | | VAKINTVDGQKRVTGVTLESGEVIE | |
| | | | ADAVVCNADLVYAYHHLLPPCSWTT | |
| | | | QTLASKKLTSSSISFYWSMSKVIEQ | |
| | | | LDVHNIFLAEAYKESFDEIFKDLSM | |
| | | | PSEASFYVNVPSRIDPSAAPANKDS | |
| | | | VIVLVPIGHMKTSQKEDYAELVKRA | |
| | | | RKMVLEIINRRLGTKDFEQYIEHEI | |
| | | | VNDPSVWQSKFNLWKGSILGLSHDV | |
| | | | FQVLWFRPSTRDSTGRYDNLYFVGA | |
| | | | STHPGTGVPIVLAGSKLTSDQVCKG | |
| | | | FGQKPLARKLQLIEKTVEPAEREKS | |
| | | | SFLLFALCFLLLVGLFAFFPEKSTG | |
| | | | HTAASFINSHLPKSLQVILDANVI | |
| SEQ | Sa. | Expanded | MVRHVIVVGSGPGGLSAAINLAGQG | Stigmatella |
| ID | CrtI_ | Sequences | LKVTVVEKDAVPGGRMKGLTLGARG | aurantiaca |
| NO: | Q096M3 | | EYALDTGPSILQLPGVLERIFVRSG | DW4/3-1 |
| 209 | | | KRIEDYVKLVPVDPNTRVHFWDGTH | |
| | | | MDTSKNLAHMEAELAKESPALPRAL | |
| | | | KAWMEEGKEKYAIAYEKFIATNAGS | |
| | | | LGYYAPWRLASTLRFKPWQTLYRHL | |
| | | | DSFFHDDRVTYALAYPSKYLGLHPT | |
| | | | TCSSVFGVIPFLELAFGVWHVDGGF | |
| | | | RALARGMKRCAEDLGATFRMGEAVE | |
| | | | QVLVDGGRAVGVRLANGKRLEGDAV | |
| | | | VVNADLAYAAQSLLPADVREGTRLS | |
| | | | DASLERAKFSCSTFMAYYGLDRVYP | |
| | | | ELPHHLIYLSENARRTDRDALEDRV | |
| | | | LDLEDPPFYVCNPCVTDPSGAPEGH | |
| | | | STLYVLVPTPNTSQPVDWAATERVL | |
| | | | RERIPDMLAKVGLKDVRRHIRAERY | |
| | | | FTAETWRDDFHVERGAVENLSHTWM | |
| | | | QLGPMRPKVKSPDVQGLYWVGGGTH | |
| | | | PGSGLLTIMESANIAADYLTREAGK | |
| | | | GPLPQWPYVPPLEEAPVTQARTG | |
| SEQ | Sg. | Expanded | MITVKGPVDHVVVVGAGLAGLAAAL | Streptomyces |
| ID | CrtI_ | Sequences | HLLGAGRRVTVVEREDVPGGRAGLL | griseus NCBI |
| NO; | P54981 | | ESGGFRIDTGPTVLTMPDLVEDAFA | 3933 |
| 210 | | | AVGERMADRLELIRLAPAYRARFAD | |
| | | | GSQLDVHTDGAAMEAAVEEFAGARQ | |
| | | | AVGYRRLRIWLERLYRVQMRRFIDT | |
| | | | NEDSPLQLAHPDLARLAALGGFGRL | |
| | | | DARIGHFVSDERLRRVESFQALYAG | |
| | | | VPPARALAAYAVIAYMDTVAGVYFP | |
| | | | RGGMHALPRAMADAASDAGAVLRYG | |
| | | | QRVTRLERSGDRVTAVVTDQEHIPC | |
| | | | DAVVLTPDLPVSYRLLGRTPHRPLP | |
| | | | LRHSPSAVILHTGTDRTWPDLAHHT | |
| | | | ISFGAAWKNTFHELTRTGRLMSDPS | |
| | | | LLITRPTATDPSLAPPGKHLHYVLA | |
| | | | PCPNTEVGPGVREWRELGPRYRDEL | |
| | | | LAELERREMPGLGAAIEEEGLVTPV | |
| | | | DWTAQGHAAGTPFSVAHTFPQTGPF | |
| | | | RPRNLVRGTVNAVLAGCGTTPGVGV | |
| | | | PTVLISGKLAAERITGPRIARAPRS | |
| | | | APGGSSA | |
| SEQ | Sr. | Expanded | MSRQKHIIVIGAGAGGTATAARLAR | Syncephalastrum |
| ID | CrtI_ | Sequences | EGYRVTVVEKNDENGGRCSLIHHNG | racemosum |
| NO: | A0A1X2H2F7 | | HRFDQGPSLYLMPKLFEECFSDLDE | |
| 211 | | | RIEDHLDLLRCDNNYKVHFDDGDSI | |
| | | | HLSSDLTRMKREAERIEGEDGESRF | |
| | | | LEFMKETHVHYERGTFLAIKRNFES | |
| | | | IFDMVKLKYAPEILRLHLEDQIYAR | |
| | | | AAKYFQTKKMRMAFTFQTMYMGMSP | |
| | | | YDAPAVYSLLQYTEFAEGIWYPRGG | |
| | | | FNMVVQKLEAIAQKSGAVFKYESPV | |
| | | | AKINTADQDRRVTGVTLQDGTVLPA | |
| | | | DAVVCNADLVYAYHKLLPPCRWVRT | |
| | | | TLASKKLTSSSISFYWSMKQKILQL | |
| | | | DVHNIFLAEAYKESFDEIFQDHTLP | |
| | | | SEPSFYVNVPSRIDSSAAPADKDSV | |
| | | | IVLVPIGYMRSQDGNCDVDYQILVK | |
| | | | RARTMVLEVLHRRLGLKNFAELIEH | |
| | | | EIVNDPSVWQQKENLWRGSILGLSH | |
| | | | DVLQVLWFRPSTKDSTGRYDNLFFV | |
| | | | GASTHPGTGVPIVLAGSKLTATQVC | |
| | | | QYFGKKGLTPSKLLSKATFEYAPEG | |
| | | | DTAFTNHLVYMLLSLFSMGMTPAAF | |
| | | | INACLPSPFRVVIAAAAEEIQI | |
| SEQ | Ss. | Expanded | MMKAVVVGAGLGGISTAILLKKFFD | Sulfolobus |
| ID | CrtI_ | Sequences | EVVVLEKNTTPGGRARYFSVGEFRF | solfataricus P2 |
| NO: | Q97UT6 | | DMGPSWYLMPEIFEEFFSEAGENPH | |
| 212 | | | PIIRVNPPVKVMESKFLKKEYREFT | |
| | | | GDNVDDRYLNKTRLFYDESKKFIKK | |
| | | | EMVFSDFLDKEVLVNISALEDTLDN | |
| | | | FNARYFRDSLMRKAMGFSSVFIGGS | |
| | | | PFRVPAVYAMINYAIYGQGVFYPYK | |
| | | | GFEGYVKRLYEIARKKGVEFKFDFP | |
| | | | VNKVKIDEGKVVEVSSNIKREMGDV | |
| | | | FVENMDYFYADSLLPEEYKVISGRR | |
| | | | YKLSPSAILAYIGLEDTLDLPHHTV | |
| | | | IINGDWREHFTSIDRNSLPSVENTS | |
| | | | YYVSYRGATNKETRDLVILIPVGTG | |
| | | | TEISKELIDGVIRDLEVKIGCKIKA | |
| | | | SYIRIYGPDDFKRDYNAYEGTAFGL | |
| | | | AHTLDQTGPFRLPMKNTRLKNLYYV | |
| | | | GQYTQPGIGVPMVTLSSMIVSKKIS | |
| | | | KEFL | |
| SEQ | Te. | Expanded | MSTQKHIVVIGAGVGGTATAARLTR | Thamnidium |
| ID | CrtI_ | Sequences | EGFRVTVVEKNSFSGGRCSLIHHNG | elegans |
| NO: | A0A8H7VTL0 | | HRFDQGPSLYLMPKMFEDAFADLDE | |
| 213 | | | RVEDHLELLRCDNNYKVHFDDGESI | |
| | | | QLSSDLTRMKSELDRIEGPLGFGRF | |
| | | | LDFLKETHIHYERGVFIAIKRNFES | |
| | | | IWDMLQVKFAPEVVRLHLFGKIYDR | |
| | | | ASKYFLTKKMRMAFTFQTMYMGMSP | |
| | | | YDAPAVYSLLQYTEFAEGIWYPKGG | |
| | | | FNMVVQKLEAIAKNKYGAKFLYDSP | |
| | | | VSKINTEGNRATGVTLDSGEFIEAD | |
| | | | AVVCNADLVYAYHHLLPPCKWTMNT | |
| | | | LAAKKLTSSSISFYWSMKEKVPKLD | |
| | | | VHNIFLAEAYQESFDEIENDHSLPS | |
| | | | EASFYVNVPSRIDPSAAPEGKDTVI | |
| | | | VLVPIGHMKSLAGNAEEADYPKMVE | |
| | | | RARAMVLEVIEHRLGMKNFSDLIEH | |
| | | | ESYNDPAIWQSKENLWRGSILGLSH | |
| | | | DIFQVLWFRPSTKDSTGRFDNLFFV | |
| | | | GASTHPGTGVPIVLAGSKLTSDQVT | |
| | | | TSFGKRPLPRKVQDQISYKAEDVTH | |
| | | | GVQLMPLFVYTFVITFLFFVFFPTN | |
| | | | ATNQTAASFINSALPKPFRVSYEND | |
| | | | LLLERLNAV | |
| SEQ | Vd. | Expanded | MQKSGEKPRKAIVVGAGAGGVAIAA | Verticillium |
| ID | CrtI_ | Sequences | RLAKAGYKVTVLEKNDFTGGRCSLI | dahliae |
| NO: | G2WRY7 | | HHEGYRFDQGPSLLLLPQLFHETER | |
| 214 | | | DLDTTIADSGVDLLRCQDVNYNVWF | |
| | | | HDGELFKHSSDLATMKVEVERWEGK | |
| | | | EGFARYLSWMREAHTHYEVSVTDVL | |
| | | | HRNFTSLENLARPSLLKHVVALHPL | |
| | | | ESIYARASRYFWTERLRRVFTFAVM | |
| | | | YMGMSPFDAPATYSLLQYTEFAEGI | |
| | | | WYPRGGFHAIIAALVRIGEQMGVEY | |
| | | | RLNTSVASISTAEESNRATGVVLES | |
| | | | GESLSADVVVINADLVYAYSNLMPK | |
| | | | TPSTVAYSQSLQKKEASCSSISFYW | |
| | | | SVNRKIPELGTHNIFLAEEYRESFD | |
| | | | AIFKRHGLPDQPSFYVNVPSRIDPS | |
| | | | AAPEDGDAIIVLVPIGHLARSKGGV | |
| | | | DAPGLPTDEKSWINLVARARAAVLE | |
| | | | TISARTGCAPIGQFITNEIVNNPLS | |
| | | | WEDKFNLDKGAILGLTHSVPNVLAF | |
| | | | RPRTRAEGLENAYFVGASTHPGTGV | |
| | | | PIVLAGAKITAEQILDDRGLPVPWA | |
| | | | HTKNFPKQQLAERRQKTKALDLMRY | |
| | | | TFHFGSEEVAVLSFLFAVCMMYLFV | |
| | | | WPRVVSLVC | |
| SEQ | Vh. | Expanded | MAHNHSEVSGRAIVIGAGFGGIASA | Vibrio harveyi |
| ID | CrtI_ | Sequences | LRFCAKGYSVTLVDKGDQLGGRARV | ATCC BAA-1116 |
| NO: | A7MVU2 | | FERNGFIFDAGPTVITAPFLIDELF | |
| 215 | | | ALFGKKTSQYVSIVPVTPWYRFYFH | |
| | | | DNTTEDYGGTLEATLEEIKKISPND | |
| | | | VIGYQNLLATSESIFDIGFTQLSDE | |
| | | | PFHNPLSMIRQVPNLIRLGCYKTVW | |
| | | | QLVCEHLEHEKLRQAFSIQPLLVGG | |
| | | | NPFDTTSIYNLIHFLELKWGVHFAI | |
| | | | GGMGALVNGLKKLMLENGIDIQLNT | |
| | | | EVKSVVSKNGKVSGIETSNGDLMDC | |
| | | | DVVVSNADPAFLYRKMIAKKDQTLS | |
| | | | ARLKSRHAKFSMGLFVLYFGTTQKY | |
| | | | EDVAHHTIWLGKRYESLLNDIFHDK | |
| | | | VLADDFSLYVHRPTATDPNMAPEGC | |
| | | | DSFYVLAPVPNLESNVDWETVKNKY | |
| | | | SDKIVNALSETLLPGLSDCIVEQFC | |
| | | | MTPSDFQCDYNSINGAGFSIAPTLT | |
| | | | QSAYFRFHNRGEGLKNLFVVGAGSH | |
| | | | PGAGLPGVLCSAKVLDKIVPEFTGD | |
| | | | DARVRSCGMPLTNTDTTKPRMNLPS | |
| | | | RLSGDNHGKYGS | |
| SEQ | Xa. | Expanded | MMLDATKTIRRAAVIGSGFGGLSLA | Xanthobacter |
| ID | CrtI_ | Sequences | IRLQAAGIRTTVFEQRDKPGGRAYV | autotrophicus |
| NO: | Q93CI7 | | YEQDGFTFDGGPTVITDPTCLEEVF | Py2 |
| 216 | | | AAAGRKLSDYVELMPVSPFYRLLWP | |
| | | | DGRQFDYVNDQAALDAQIATEDKAD | |
| | | | VEGYRRFLAYSQAVFEEGYLKLGAV | |
| | | | PFLEFSSMMKAAPKLVRLEAWRSVY | |
| | | | AMVSRFIRDDHLRQAFSFHSLLVGG | |
| | | | NPESTSSIYALIHALERKWGVFFPR | |
| | | | GGTGALVRGMVKLFTDLGGEIRLTS | |
| | | | PVDEIVVEGQRATAVKLKSGETLPF | |
| | | | DLVASNADVVHTYRHLLRGAARGRS | |
| | | | EGARLAKTRHSMSLFVTYFGARRTW | |
| | | | DHLQHHTVLFGPRYRGLVDEIFKGP | |
| | | | NLPDDFSLYLHAPTVTDKSLAPEGC | |
| | | | TAFYVLSPVPHLGKADIDWEVEGPL | |
| | | | YRDRILAHLEERLLPGLRDSLVTSR | |
| | | | ILTPFGERDELSAHQGSAFSVEPLL | |
| | | | TQSAWFRPHNRDAKIANLYFAGAGT | |
| | | | HPGAGVPGVVGSAKATAGLILADLG | |
| | | | VTPR | |
| SEQ | Mc. | Original | MLLTYMEVHLYYTLPVLGVLSWLSR | Mucor |
| ID | CrtYB_ | filing | PYYTATDALKFKFLTLVAFTTASAW | circinelloides |
| NO: | Q9UUQ6 | | DNYIVYHKAWSYCPTCVTAVIGYVP | |
| 153 | | | LEEYMFFIIMTLLTVAFTNLVMRWH | |
| | | | LHSFFIRPETPVMQSVLVRLVPITA | |
| | | | LLITAYKAWHLAVPGKPLFYGSCIL | |
| | | | WYACPVLALLWFGAGEYMMRRPLAV | |
| | | | LVSIALPTLFLCWVDVVAIGAGTWD | |
| | | | ISLATSTGKFVVPHLPVEEFMFFAL | |
| | | | INTVLVFGTCAIDRTMAILHLFKNK | |
| | | | SPYQRPYQHSKSFLHQILEMTWAFC | |
| | | | LPDQVLHSDTFHDLSVSWDILRKAS | |
| | | | KSFYTASAVFPGDVRQELGVLYAFC | |
| | | | RATDDLCDNEQVPVQTRKEQLILTH | |
| | | | QFVSDLFGQKTSAPTAIDWDFYNDQ | |
| | | | LPASCISAFKSFTRLRHVLEAGAIK | |
| | | | ELLDGYKWDLERRSIRDQEDLRYYS | |
| | | | ACVASSVGEMCTRIILAHADKPASR | |
| | | | QQTQWIIQRAREMGLVLQYTNIARD | |
| | | | IVTDSEELGRCYLPQDWLTEKEVAL | |
| | | | IQGGLAREIGEERLLSLSHRLIYQA | |
| | | | DELMVVANKGIDKLPSHCQGGVRAA | |
| | | | CNVYASIGTKLKSYKHHYPSRAHVG | |
| | | | NSKRVEIALLSVYNLYTAPIATSST | |
| | | | THCRQGKMRNLNTI | |
| SEQ | Mc. | Original | MLLTYMEVHLYYTLPVLGVLSWLSR | Mucor |
| ID | CrtYB_ | filing | PRYTATDALKFKFLTLVAFTTASAW | circinelloides |
| NO: | Q9UUQ6_ | | DNYIVYHKAWSYCPTCVTAVIGYVP | |
| 154 | Y27R | | LEEYMFFIIMTLLTVAFTNLVMRWH | |
| | | | LHSFFIRPETPVMQSVLVRLVPITA | |
| | | | LLITAYKAWHLAVPGKPLFYGSCIL | |
| | | | WYACPVLALLWFGAGEYMMRRPLAV | |
| | | | LVSIALPTLFLCWVDVVAIGAGTWD | |
| | | | ISLATSTGKFVVPHLPVEEFMFFAL | |
| | | | INTVLVFGTCAIDRTMAILHLFKNK | |
| | | | SPYQRPYQHSKSFLHQILEMTWAFC | |
| | | | LPDQVLHSDTFHDLSVSWDILRKAS | |
| | | | KSFYTASAVFPGDVRQELGVLYAFC | |
| | | | RATDDLCDNEQVPVQTRKEQLILTH | |
| | | | QFVSDLFGQKTSAPTAIDWDFYNDQ | |
| | | | LPASCISAFKSFTRLRHVLEAGAIK | |
| | | | ELLDGYKWDLERRSIRDQEDLRYYS | |
| | | | ACVASSVGEMCTRIILAHADKPASR | |
| | | | QQTQWIIQRAREMGLVLQYTNIARD | |
| | | | IVTDSEELGRCYLPQDWLTEKEVAL | |
| | | | IQGGLAREIGEERLLSLSHRLIYQA | |
| | | | DELMVVANKGIDKLPSHCQGGVRAA | |
| | | | CNVYASIGTKLKSYKHHYPSRAHVG | |
| | | | NSKRVEIALLSVYNLYTAPIATSST | |
| | | | THCRQGKMRNLNTI | |
| SEQ | Nc. | Original | MYDYAFVHLKFTVPAAVLLTAIAYP | Neurospora |
| ID | CrtYB_ | filing | ILNRIHLIQTGFLVVVAFTAALPWD | crassa |
| NO: | P37295 | | AYLIKHKVWSYPPEAIVGPRLLGIP | |
| 156 | | | FEELFFFVIQTYITALVYILFNKPV | |
| | | | LHALHLNNQQNPPAWMRVVKVTGQV | |
| | | | VLVALSVWGWNAAQVHQETSYLGLI | |
| | | | LVWACPFLLAIWTLAGRFILSLPWY | |
| | | | ATVLPMFLPTFYLWAVDEFALHRGT | |
| | | | WSIGSGTKLDFCLFGKLDIEEATFF | |
| | | | LVTNMLIVGGMAAFDQYLAVIYAFP | |
| | | | TLFPKVNRYPTTHMLLQSRLINTSR | |
| | | | YDLERIEGLREAVERLRLKSRSFYL | |
| | | | ANSLFSGRLRIDLILLYSFCRLADD | |
| | | | LVDDAKSRREVLSWTAKLNHFLDLH | |
| | | | YKDADATEDPKKKAERIDAYIKTAF | |
| | | | PPCAYQALHLLPTHILPPKPLYDLI | |
| | | | KGFEMDSQFTFHGTSDSTDLQYPIA | |
| | | | DDKDLENYAIYVAGTVGELCIALII | |
| | | | YHCLPDMSDTQKRELETAACRMGIA | |
| | | | LQYVNIARDIVVDARIGRVYLPTTW | |
| | | | LKKEGLTHKMVLENPEGPEVIERMR | |
| | | | RRLLENAFELYGGARPEMQRIPSEA | |
| | | | RGPMIGAVENYMAIGRVLRERKEGT | |
| | | | VFVRMEGRATVPKRRRLSTLLRALY | |
| | | | EQ | |
| SEQ | Pb. | Original | MLTYMEVHLYFTLPVLALLAFLYKP | Phycomyces |
| ID | CrtYB_ | filing | FFTTKDRFKYIFLCTVAFATASPWD | blakesleeanus |
| NO: | Q9P854 | | NYIVYHKAWSYCPECVTAVIGYVPL | |
| 155 | | | EEYMFFIIMTLITVTFTSLTMRWTL | |
| | | | PSFFIRPETPVFQSVCVRYIPIVGF | |
| | | | LTIAAKAWASSIPDSHPFYGACILW | |
| | | | YVCPVLALLWIGSGEYMLRRWKAVL | |
| | | | FSIAVPTIFLCWVDQYAIARGTWDI | |
| | | | SRRTSTGIMVLPSLPLEEFLFFLLI | |
| | | | DTVLVFASCATDRAHAIVHIYITPM | |
| | | | NHNKVSTWYMDFFYLCWAFLQTDQA | |
| | | | LSGETLSDLDATWRILREASASFYT | |
| | | | ASSVFSFEARQDLGVLYGFCRATDD | |
| | | | LADNNDVSVPDRKKQLELVRGFVRQ | |
| | | | MFDSKHGHPDIDWTQYSGSIPDSFI | |
| | | | AAFRSFTRLRDVLEIKAVEELLDGY | |
| | | | TFDLEQREVKNEDDLVYYSACVASS | |
| | | | VGEMCTRVLMASEPGGNRTMLKWTV | |
| | | | ERARDMGLALQLTNIARDIVTDSKQ | |
| | | | LGRSYVPRDWLTSQESALLKAGKAR | |
| | | | ELGDERLRQIALKMVYTADDLNLMA | |
| | | | SRAIDYLPPSSRCGVRAACNVYTAI | |
| | | | GVSLHKANGYPDRAHLTKLERMKVT | |
| | | | FRCVYGFRKGHQGVQGDRGKSQAFT | |
| | | | VI | |
| SEQ | Xd. | Original | MTALAYYQIHLIYTLPILGLLGLLT | Phaffia |
| ID | CrtYB_ | filing | SPILTKFDIYKISILVFIAFSATTP | rhodozyma |
| NO: | Q7Z859 | | WDSWIIRNGAWTYPSAESGQGVFGT | (Xanthophyllomyces |
| 10 | | | FLDVPYEEYAFFVIQTVITGLVYVL | dendrorhous) |
| | | | ATRHLLPSLALPKTRSSALSLALKA | |
| | | | LIPLPIIYLFTAHPSPSPDPLVTDH | |
| | | | YFYMRALSLLITPPTMLLAALSGEY | |
| | | | AFDWKSGRAKSTIAAIMIPTVYLIW | |
| | | | VDYVAVGQDSWSINDEKIVGWRLGG | |
| | | | VLPIEEAMFFLLTNLMIVLGLSACD | |
| | | | HTQALYLLHGRTIYGNKKMPSSFPL | |
| | | | ITPPVLSLFFSSRPYSSQPKRDLEL | |
| | | | AVKLLEEKSRSFFVASAGFPSEVRE | |
| | | | RLVGLYAFCRVTDDLIDSPEVSSNP | |
| | | | HATIDMVSDFLTLLFGPPLHPSQPD | |
| | | | KILSSPLLPPSHPSRPTGMYPLPPP | |
| | | | PSLSPAELVQFLTERVPVQYHFAFR | |
| | | | LLAKLQGLIPRYPLDELLRGYTTDL | |
| | | | IFPLSTEAVQARKTPIETTADLLDY | |
| | | | GLCVAGSVAELLVYVSWASAPSQVP | |
| | | | ATIEEREAVLVASREMGTALQLVNI | |
| | | | ARDIKGDATEGRFYLPLSFFGLRDE | |
| | | | SKLAIPTDWTEPRPQDEDKLLSLSP | |
| | | | SSTLPSSNASESFRFEWKTYSLPLV | |
| | | | AYAEDLAKHSYKGIDRLPTEVQAGM | |
| | | | RAACASYLLIGREIKVVWKGDVGER | |
| | | | RTVAGWRRVRKVLSVVMSGWEGQ | |
| SEQ | Ag. | Expanded | MGLDYLMVHVKYNLPPALLLTILYK | Arthroderma |
| ID | CrtYB_ | Sequences | PFFTRLEVHKIVLLCTIAVVWTIPW | gypseum |
| NO: | E4UPP6 | | DSYLIRTRIWSYPADSVLGQTLFQI | |
| 217 | | | PLEEVFFFIIQTYNTSLLYIIFNKR | |
| | | | LVLPSYLSGPTKPLAQGLFGPITHR | |
| | | | TQRDLGTLFFTGILILGISFIYIGG | |
| | | | EYMYLGLILSWVSPILVMQWVLMYR | |
| | | | FLLALPPASVWVPIALPTLYLWVVD | |
| | | | TLALRRGTWVIESGTKVDIQLWEGL | |
| | | | EIEEALFFLVTNVMVVFGIAAMHNA | |
| | | | AALFEYKAFISTTAMGDTPSIYQLI | |
| | | | TLFLTSSRLYDTNVLQEMSQAVTLL | |
| | | | KQKSQSMYLGSAMFEGQLRLDLIAL | |
| | | | YSFCRKADDLIDDAPDRETAKYWIE | |
| | | | QCEKALELRFKLKETALDDTEAYQL | |
| | | | LTKSIPQPLHAAAHLLPASRLPKEP | |
| | | | LSCLLQGFEIDLKFDFEKGSFPIAT | |
| | | | EHDLEVYAYHVAGTVASLLLELVER | |
| | | | HHPVSISEAERLRVISAGEVMGRAL | |
| | | | QYTNIARDITRDAEIGRVYIPSSWL | |
| | | | AEEGLTPSMVISHPRNSKLIPLRRR | |
| | | | ILEKADKCYCETQEAISKLPSNVQG | |
| | | | PVRATVTAYMEIGQVIRENETKIWN | |
| | | | GKLKVSRWRRFKRAWLAML | |
| SEQ | An. | Expanded | MNQNGTRLCYSVREVPPTSFVSAPA | Aspergillus |
| ID | CrtYB_ | Sequences | NYKRASSHCTYTIPAASALTVLYYP | niger |
| NO: | A2QM49 | | FFTAQDRCKICILITIAILATLPWD | |
| 218 | | | SYLIRSAIWTYPPDAVVGLKILDIP | |
| | | | IEEVFFFAIQTYITSLTYCIFTKPL | |
| | | | VRPMYLRSHLERRGTRYVVATVILA | |
| | | | LMGGGTACLLLGRRMTYLGLILVWA | |
| | | | CPILLFQWMMSYPFLSELPWKPTIT | |
| | | | SICLPTLHLWFADSRAMGTGTWRIE | |
| | | | EGTKLNFRIGGLELEEALFFLVSNM | |
| | | | MVVLGLVGCDYAYALQEYESLSQPA | |
| | | | SDVYITLRKALSLLARPLPIDASLI | |
| | | | SALSQAVYRLQEKSQSMFLGSALFQ | |
| | | | GQLRIDLIFLYSFCRVMDDLIDEAE | |
| | | | DEQEARFWVTECRHLLDSTHRSEPH | |
| | | | SDHFYTGKKGEEHERLRQSISYLPP | |
| | | | SHLSNDSFDDLLKGFEIDLKFNPQR | |
| | | | EAFPIQSEYCLDQYAGFVAGTVGVL | |
| | | | VEDLTLFHCGHYFIQDVPRLRRAAK | |
| | | | DMGKAMQCQPRGDGGATSGRKRAIA | |
| | | | GNRGELYGYWTTAEGAERHKPGASV | |
| | | | EDEGAFGATAKSWLAGDVITELCHC | |
| | | | FIQAEYVIYLVRHQNASADTLVLLS | |
| | | | ALVYRLE | |
| SEQ | Ao. | Expanded | MGLDYILVHVTYNIPLAGILTLVYW | Aspergillus |
| ID | CrtYB_ | Sequences | PFMTRLDWQKISTLVIISLVATIPW | oryzae |
| NO: | Q2U4X9 | | DSYLVRHRIWTYAPNGVIGWTLYDI | |
| 219 | | | PSEEVFFFIIQTYNTSLVYLILTRW | |
| | | | LVLPMYLGTVARKETLIGASILLLA | |
| | | | ISVGLIALCFGDHFTYFGMIITWAG | |
| | | | PFLLIQWVFSSGFIIALPKLELMVS | |
| | | | ITLPTLFLWTVDTISINQGTWTVEA | |
| | | | PTKLGVQLWSGMDIEEVLFFLITNI | |
| | | | VIVFGLVCIDYAIAMATCELVQSPQ | |
| | | | AVQSFPSYFRVLARFVTNKYHPDKQ | |
| | | | FVASLRKAVDRLAASSQSMYMGSAM | |
| | | | FQGPFRIDLILLYSFFRVADDLVDE | |
| | | | SQDTESARMIIEQCDQLLEAKFSHP | |
| | | | ELFPFSPGYQEAKHPAPPELIAAID | |
| | | | SLPVSRLRLEHLKGLIEGFRTDLTF | |
| | | | SAKPGSFPFVTESDLDTYAYHVASS | |
| | | | VAASMLGLVVHHFPDHQFAINVELR | |
| | | | RRVVDAGERMGQTLQYINVARDIAR | |
| | | | DAAINRVYLPTTWLKQQGLGPEDVL | |
| | | | ASPTDSRLELVRDRLLDRAEFLSAS | |
| | | | AREEMKFLPDEVQGPFLATVDSYLE | |
| | | | IGAALRRGMRPRTLDDKLRLPLGTR | |
| | | | LWVAYRAMAWRK | |
| SEQ | Bt. | Expanded | MSILTYLEFHLYYTLPVLAALCWLL | Blakeslea |
| ID | CrtYB_ | Sequences | KPFHSQQDNLKYKFLMLMAASTASI | trispora |
| NO; | Q67GH9 | | WDNYIVYHRAWWYCPTCVVAVIGYV | |
| 220 | | | PLEEYMFFIIMTLMTVAFSNFVMRW | |
| | | | HLHTFFIRPNTSWKQTLLVRLVPVS | |
| | | | ALLAITYHAWHLTLPNKPSFYGSCI | |
| | | | LWYACPVLAILWLGAGEYILRRPVA | |
| | | | VLLSIVIPSVYLCWADIVAISAGTW | |
| | | | HISLRTSTGKMVVPDLPVEECLEFT | |
| | | | LINTVLVFATCAIDRAQAILHLYKS | |
| | | | SVQNQNPKQAISLFQHVKELAWAFC | |
| | | | LPDQMLNNELEDDLTISWDILRKAS | |
| | | | KSFYTASAVFPSYVRQDLGVLYAFC | |
| | | | RATDDLCDDESKSVQERRDQLDLTR | |
| | | | QFVRDLFSQKTSAPIVIDWELYQNQ | |
| | | | LPASCISAFRAFTRLRHVLEVDPVE | |
| | | | ELLDGYKWDLERRPILDEQDLEAYS | |
| | | | ACVASSVGEMCTRVILAQDQKENDA | |
| | | | WIIDRAREMGLVLQYVNIARDIVTD | |
| | | | SETLGRCYLPQQWLRKEETEQIQQG | |
| | | | NARSLGDQRLLGLSLKLVGKADAIM | |
| | | | VRAKKGIDKLPANCQGGVRAACQVY | |
| | | | AAIGSVLKQQKTTYPTRAHLKGSER | |
| | | | AKIALLSVYNLYQSEDKPVALRQAR | |
| | | | KIKSFFVD | |
| SEQ | Cg. | Expanded | MGYDYALVHVKYTIPLAALLTVESY | Colletotrichum |
| ID | CrtYB_ | Sequences | PVFTRLDVVRTLFIVTIAFVATIPW | graminicola |
| NO: | E3Q717 | | DSYLIRTNVWTYPPDAVLGPTLYDI | |
| 221 | | | PAEELFFFIIQTYITAQLYIILNKP | |
| | | | VLHAQYLNSPATLPQWIKSGKLVGQ | |
| | | | LALSGSVLLGTWLIAKKGEGTYLGL | |
| | | | ILVWACTFALFTWTITAHFLLALPL | |
| | | | ACTALPILLPTVYLWIVDEMALGRG | |
| | | | TWAIESGTKLELQLFGSLEIEEATF | |
| | | | FLVTNMLIVFGIAAFDKAVAVCDAF | |
| | | | PEKFDKPADALAMSLLRARVFPSSK | |
| | | | YDMQRILGIRQAAARLAKKSRSFHL | |
| | | | ASSVFPGRLRIDLTLLYSYCRLADD | |
| | | | LVDDAATPEEAAVWISKLDRHLSLL | |
| | | | YKDPDATSTPLASKYAAENFPPSAL | |
| | | | SALDMLPTSLLPREPLAELLKGFEM | |
| | | | DLSFSNSAFPIADPEDLELYAARVA | |
| | | | STVGQACLELVFCHCQHGLPDYMKA | |
| | | | YLRNTARQMGLALQFVNISRDIAVD | |
| | | | AKIGRVYLPTTWLKEEGLTPEDVLK | |
| | | | SPNSEGVGKVRRRILAKALDHYGEA | |
| | | | RDSMKWIPSEARGPMIVAVESYMEI | |
| | | | GRVLMRNGGSAAADGSGRATVPKSR | |
| | | | RIWVAWSTLMAA | |
| SEQ | Gf. | Expanded | MGWEYAQVHLKYTIPFGVVLAAVYR | Gibberella |
| ID | CrtYB_ | Sequences | PLMSRLDVFKLVFLITVSFFWVVKG | fujikuroi (F. |
| NO: | Q8X0Z1 | | LEANSCRLLLFLPCMLILRGVLLRL | fujikoroi) |
| 222 | | | TNADSPWDSYLIKNRIWTYPPGVVV | |
| | | | GLTAWDIPAEELFFFVIQTENTSLL | |
| | | | YMILSKPTFHPIYLSKKTGWGKIAG | |
| | | | QILFASAIIFGLVSVSSGGEGMYMG | |
| | | | LILIWACPFLLFLWSISYQFIVNLP | |
| | | | WTNTALPIALPTLYLWVVDTFALRR | |
| | | | GTWSITSGTKYGVVLWDGLEIEEAV | |
| | | | FFLLTNTLIVEGLIACDNNLAILDT | |
| | | | FPEHFPRTKGVPSLLTIIRTLILPK | |
| | | | EKYDEERIQGLVSAVALLRKKSRSF | |
| | | | YLASGTFEGRLRIDLIRLYAFCRAA | |
| | | | DDLVDEAPSVDDSRASIEKLRKELD | |
| | | | LAYEENQEEPSQRLREYVTSSIPEM | |
| | | | FHMALLQLPTYYLPKQPLDDLLKGF | |
| | | | DTDLLFDRKSGAFPIETTEDLDIYG | |
| | | | SRVAGTVAELCNHLILYHTPEAVPE | |
| | | | DIQREVVASGQEMGIALQYVNIARD | |
| | | | IKTDAEIDRVYLPLSWLKEAQLTPE | |
| | | | DVIQQPHGPTIEALRHKLLDRAFEK | |
| | | | YNMAKGAIDKLPSEGKGPIRVAVES | |
| | | | YMEIGRVLREKGPTMKKGRATVPKM | |
| | | | RRIRVAWSALNK | |
| SEQ | Lm. | Expanded | MGFDYALVHLKYTIPPAVLLTLLYR | Leptosphaeria |
| ID | CrtYB_ | Sequences | PLLTKIDVYKVAFLVTIAVVATIPW | maculans |
| NO: | E4ZUB5 | | DSYLIRNRIWSYPDHVIIGPTLFDI | |
| 223 | | | PLEEVFFFVVQTYNTSLLYLVLSKP | |
| | | | TFQPVYLCTERDELHGSWRLKRLIG | |
| | | | QAILLGAIAWGWFCVRERGLGTYTG | |
| | | | LILIWAGPFLLLLWSLAYQFIIGLP | |
| | | | FTNTLLPIVLPTLYLWIVDTLALRR | |
| | | | GTWVISPGTKFGVHLWDGLEIEEAL | |
| | | | FFLLTNVLIVFGQLAFDNALAVLYA | |
| | | | FPHLFPDPSLLPSPATLIRSLLTSC | |
| | | | AQYDEARLTGFREAVSRLKRKSRSF | |
| | | | YLASSTFQGPLRMDLLLLYSFCRVA | |
| | | | DDLVDNAATTEEARQWIAKLHKELD | |
| | | | NVYRKDVVCSSVTDQVRKEFPLDTH | |
| | | | SALLQLPCCKLSAEPLRDLLRGFEM | |
| | | | DLEFNSTSPIQSTEDLVLYSERVAG | |
| | | | TVAQMCIQLIFHLYPSSLTAEKRHK | |
| | | | VVAAGNSMGVALQYVNIARDIGVDA | |
| | | | KIGRVYLPTDWLSEVGLNCDTVLKD | |
| | | | PKDPRIEALRGRLLDDAFSFYEEAK | |
| | | | LAIAQLPIEAQGPIRVAVESYMEIG | |
| | | | RTLKQDGFIVKAGRATVPKWRRVLV | |
| | | | AWRTLN | |
| SEQ | Ma. | Expanded | MEVHLYYTLPMLGVIFWLSKPYYTS | Mucor ambiguus |
| ID | CrtYB_ | Sequences | TDSLKFKFLSLVAFTTASVWDNYIV | |
| NO: | A0A0C9MMT4 | | YHKAWSYCPTCVTAVIGYVPLEEYM | |
| 224 | | | FFIIMTLLTVAFTNLVMRWHLHSFF | |
| | | | IKPETPIMQSVLVRFVPITALLTTA | |
| | | | YKAWHSAVPGNPLFYGSCILWYACP | |
| | | | VLALLWFGAGEYMMRRPLAVLSSIA | |
| | | | LPTLFLCWVDVVAIGAGTWDISLAT | |
| | | | STGIFVVPHLPVEEFMFFALINTVL | |
| | | | VFGTCAIDRTMAIIHLFKKKSPYQR | |
| | | | QYQNDKSFLHQILEMTWAFCLPDQA | |
| | | | LHTETFHDLSISWDILRKASRSFYT | |
| | | | ASAVFPGDVRQELGVLYAFCRATDD | |
| | | | LCDNEQVPVQERKDQLTLTHRFVSD | |
| | | | LESQKKSAPTAIDWDFYNDQLPAPC | |
| | | | ISAFKSFTRLRHVLEADAIKELLDG | |
| | | | YKWDLERRCITNQEDLNYYSACVAS | |
| | | | SVGEMCTRIILAHADKPTSRQETQW | |
| | | | IIQRAREMGLVLQYTNIARDIVTDS | |
| | | | KELGRCYLPQDWLADKEVGLIQDGR | |
| | | | AREIGEERLLSLSHRLIYQADELMA | |
| | | | VANKGIDKLPSHCQGGVRAACNVYA | |
| | | | SIGTKLKSYRHHYPSRAHVGNSKRV | |
| | | | QIALLSVYNLYTAPIVTKQGRQGKM | |
| | | | RNLNTI | |
| SEQ | Mc. | Expanded | MLTYMEVHLYYTLPILGVLFGLLRP | Mucor |
| ID | CrtYB_ | Sequences | FHSPQETFKYTFLCAMAFSTASIWD | circinatus |
| NO: | A0A8H7S7S8 | | NYIVYHRAWSYCPSCVTAVIGYVPL | |
| 225 | | | EEYMFFIIMSLMTVAFTNMIMRWHL | |
| | | | PSTFIKPDTPMLKSMLVRYLPIVGF | |
| | | | LSVGVHAWNLAVPDTHLFYGSCILW | |
| | | | YSCPVLALLWEGGGEYICRRYKAVL | |
| | | | TSIIVPTIYLCWVDKVAIAAGTWHI | |
| | | | SLRTSTGVMVAPSLPLEEFMFFLLI | |
| | | | NVVLVFATCAIDRAQAILHLHGNEL | |
| | | | RGHPNNSKLSNLIPNQLTQLLYLTQ | |
| | | | AFCMSDQSLDTETYRDLHTTWKILK | |
| | | | QASFSFYTASAVFPTHIRQDLGVLY | |
| | | | GFCRATDDLADDEVASVEQRKTQLA | |
| | | | TVRRCIHDMFATKTPNMDWSLYDLP | |
| | | | SSCIASLRAFTRLRPKLQVDAVNEL | |
| | | | LDGYDFDLDRSIVKNEADLERYSAC | |
| | | | VASSVGEMCTRVMLEYPETWTIERA | |
| | | | RDMGLVLQYTNIARDIVTDSQQLRR | |
| | | | CYVPRDWLTEKESEAMTTGRAEQLG | |
| | | | QTKLRSLALKLVYSADELATRARRG | |
| | | | IDRLPFDCQGGVRAACSVYMAIGNE | |
| | | | LIKADGYPHRAHVSTWNRAWVVLKN | |
| | | | VYQEAHSSSLQGFIRNSTSITNSKN | |
| | | | NSVDDENKIATTRKGKQRAFCLL | |
| SEQ | Mc. | Expanded | MLLTYMEVHLYYTLPMLGVLSWLSR | Mucor |
| ID | CrtYB_ | Sequences | PYYTSTDSLKFKFLSLVAFTTASVW | circinelloides |
| NO: | S2JTE4 | | DNYIVYHKAWSYCPTCVTAVIGYVP | f. |
| 226 | | | LEEYMFFIIMTLLTVAFTNLVMRWH | circinelloides |
| | | | LHSFFIKPETSVMQSVLVRFVPITA | |
| | | | LLIIAYKAWHLAVPGKPLFYGSCIL | |
| | | | WYACPVLALLWFGAGEYMLRRPLAV | |
| | | | ITSIALPTLFLCWVDVVAIGAGTWD | |
| | | | ISLATSTGKFVVPHLPVEEFMFFAL | |
| | | | INTVLVFGTCAIDRTMAILHLFKKK | |
| | | | SPYQRQYQREKTFVHQILEMTWAFC | |
| | | | LPDQVLNNETFHDLSVSWDILRKAS | |
| | | | KSFYTASAVFPGDVRQELGVLYAFC | |
| | | | RATDDLCDNEDVPVQKRKDQLSTTH | |
| | | | QFVSDLFSQKSSAPTAIDWDFYNDQ | |
| | | | LPASCISAFKSFTRLRHVLEADAIK | |
| | | | ELLNGYKWDLERRSIMNQEDLRYYS | |
| | | | ACVASSVGEMCTRIILARSDKAYSQ | |
| | | | QETQWIIQRAREMGLVLQYTNIARD | |
| | | | IVTDSKELGRCYLPQDWLTEKEIGL | |
| | | | IQNGRAREIGEARLLSLSHRLVYQA | |
| | | | DELMVVANKGIDRLPSHCQGGVRAA | |
| | | | CNVYASIGTKLKSYKQHYPSRAHVG | |
| | | | NSKRVQIALLSVYNLYTAPIAIKQG | |
| | | | RQGKMRNLNTI | |
| SEQ | Mp. | Expanded | MVLTYMEVHLYYTLPMLGLLSWLSK | Mucor plumbeus |
| ID | CrtYB_ | Sequences | PYYTSNDSLKFKFLTLIAFTTASVW | |
| NO: | A0A8H7RIZ6 | | DNYIVYHNAWSYCPTCVTAVIGYIP | |
| 227 | | | LEEYMFFIIMTLLTVAFTNLIMRWH | |
| | | | LHSFFIKPETPILQSILVRFIPITG | |
| | | | LLITAYKAWYLAVPGKSLFYGSCIL | |
| | | | WYACPVLALLWFGAGEYMLRRPLAV | |
| | | | FVSIALPTLFLCWVDVVAIGAGTWD | |
| | | | ISLATSTGKFVVPHLPVEEFMFFAL | |
| | | | INTVLVFGTCAIDRTMAILHLERKK | |
| | | | NPYQSKYQQTEKTFLHQIVEMTWAF | |
| | | | CLPDQALNTETFKDLSVSWDILRKA | |
| | | | SKSFYTASAVFPGDIRQELGVLYAF | |
| | | | CRATDDLCDDESVSVNERKKQLKLT | |
| | | | SQFVSDLFSQKVSAPSAISWEIYSD | |
| | | | QLPASCISAFKSFTCLRHVLEVEAI | |
| | | | KELLNGYKWDLERRSVLNQDDLRQY | |
| | | | SACVASSVGEMCTRIILAHSDKFYT | |
| | | | QQETQWIIQRAREMGLVLQYTNIAR | |
| | | | DIVTDSKKLGRCYLPHDWLSENDIV | |
| | | | MIQNGYAREIGEEKLLLLSHRLIQQ | |
| | | | ADELMLIANKGIEKLPSHCQGGVRA | |
| | | | ACNVYASIGAKLKLHKQQYPSRAHV | |
| | | | SNLKRIKIALFSVYNLYNAPTTIKQ | |
| | | | NRQEKM | |
| SEQ | Ms. | Expanded | MLTYMEVHLYFTLPVVGLLYFILKP | Mucor |
| ID | CrtYB_ | Sequences | FHSKQDNLKYQFLTAVAVTTASLWD | saturninus |
| NO: | A0A8H7RF36 | | NYIVYHKAWSYCPTCVVAVIGYVPL | |
| 228 | | | EEYMFFVIQTLMTVSFSNLVMRWHL | |
| | | | PTFYIKPQVSVYQSLFVRFIPIFGL | |
| | | | LTIAYKAWNVAIPGKSLFYGGCILW | |
| | | | YVCPVLALLWYGAGEYILRRPLAVL | |
| | | | TSIVVPTIYLCWVDRYAIRAGTWDI | |
| | | | SLQTSTGKMVVPHLPLEEFMFFALI | |
| | | | NTVLVFAACAIDRAQAILHLFKTES | |
| | | | VYKTHTQAPLVQRMSELCWAFCLPD | |
| | | | QMLNNETLQDLSISWDILKKASKSF | |
| | | | YTASAVFSSYARQDLGILYGFCRAT | |
| | | | DDLCDDESVSVEKRKEQLNLTRRFV | |
| | | | QDIFSQKTNMPVVDWEFYNRQLPSS | |
| | | | CVSAFKTFTRLRHILHVDAIEELLE | |
| | | | GYEWDLERKEVRTQDDLYKYSACVA | |
| | | | SSVGEMCTRILLFHDYRQSDVWIIA | |
| | | | RAREMGLVLQLTNIARDIVTDSQEL | |
| | | | GRCYLPMDWLTDNESSLIAAGKARE | |
| | | | LGDSRLLQLSKTLIRDAESIMKTAN | |
| | | | KGIEALPFHCRGGVRAACNVYAAIG | |
| | | | HVILKERTHYPTRAHIKSSGRSKIA | |
| | | | LLSVYDLYKHKTEAPKSRQGKIRGE | |
| | | | MV | |
| SEQ | Pa. | Expanded | MAYDYALVHLKYTIPLAALLTVIAY | Podospora |
| ID | CrtYB_ | Sequences | PIFHRIHFLQIGSLIVVSFLATLPW | anserina |
| NO: | B2ATB0 | | DSYLIRSNIWTYPPDAIIGPRLYGI | |
| 229 | | | PIEELFFFVIQTYITSLFYILLSKP | |
| | | | LFHPLYLSTQRNPPQRIARGKVIGQ | |
| | | | GILVALTLYGVHQIRTGGPGTYLGL | |
| | | | ILAWAFPFALLTFTVAGRFILTLPL | |
| | | | TSTVVPIIIPTVYLWLVDELALGRG | |
| | | | TWAIESGTKLGWCLFGVLDIEEATF | |
| | | | FLATNILIVFGMAVEDQYLAIIFAF | |
| | | | PHLFPKVPRSPTPLMLVQSRESNTK | |
| | | | QYDLERIAGLSDAVTRLKAKSRSFY | |
| | | | LANSLFTGRLRIDLILLYSFCRLAD | |
| | | | DLVDDSTSRTEVKSWTTKLYKFLDL | |
| | | | HYKSDVKANKARINDYIDEAFPPEA | |
| | | | KSALKYLPATILPSQPLYQLIEGFE | |
| | | | LDSQFSFHDSSESAKYPIVDEDKLN | |
| | | | YYGQCVAGTVGELCVALIIEHCEPE | |
| | | | MPDERKKMLMSVSRTMGVALQYVNI | |
| | | | ARDIVVDAEMGRVYLPTTWLKEEGL | |
| | | | TPEDVVAHPRGKHVENLRRRLLSEA | |
| | | | FKLYDEARPKMNGIPKEARGPMIGA | |
| | | | VETYMEIGRVLRELEGGVELERGKA | |
| | | | TVPGGRRLKTVLKALFSA | |
| SEQ | Pn. | Expanded | MGFDYAIVHVKYTIPPAVLLTLLYR | Phaeosphaeria |
| ID | CrtYB_ | Sequences | PLFTKLDAFKVLFLVTVAVTATIPW | nodorum |
| NO: | Q0V6M5 | | DSYLIRTNIWSYPDHVVIGPTLLDI | |
| 230 | | | PLEEVFFFFIQTYNTTLLYLILSKP | |
| | | | TFQPAYLRAGRPTASSPWKYQKLAG | |
| | | | QLFLVGATVWAGLRVHENAKGTYTG | |
| | | | LIVVWAAPIILLQWTLAYQFILGLP | |
| | | | WINTVLPIAIPTLYLWLVDTLALRR | |
| | | | GTWVISPGTKYGVHLWDGLEIEEAL | |
| | | | FFFVTNTLIVFGQLAFDNALAVLYT | |
| | | | FPALFPKPPSMPTPLDLINALWVSP | |
| | | | YKYDRARLAGLQDAVLRLKRKSRSF | |
| | | | YLASATFPGPLRSDLLLLYSFCRVA | |
| | | | DDLVDNAATAEEAKEWISKLHQYLD | |
| | | | LVYSDAKSSTVSEDFVQAHFPSDAR | |
| | | | SALLQLPAHKLPRQPLQDLLHGFEM | |
| | | | DLAFNTSSPIKTETDLRLYSERVAG | |
| | | | TVAQMCIELIFRLYPSNMTSGEERK | |
| | | | VVDAGNQMGMALQYVNIARDISVDA | |
| | | | HIGRVYLPLDWLQESGLTYDEVLIS | |
| | | | PEGARMESLRMRLLEKAFSIYDGAR | |
| | | | GAIETLPVEARGPIRVAVESYMEIG | |
| | | | RTLRQKGYTVRAGRATVSKWRRVIV | |
| | | | AWRTLNKSIA | |
| SEQ | Pt. | Expanded | MGFDYALVHLKYTIPPAVLLTWLYR | Pyrenophora |
| ID | CrtYB_ | Sequences | PFFTKLDVYKVGYLVSIAVASTIPW | tritici- |
| NO: | B2WAQ3 | | DSYLIRTGIWSYPTHVIIGPKLCDI | repentis |
| 231 | | | PLEEVFFFIIQTYNTSLLYLLLSRP | |
| | | | TFQPVYLNTERGAARRQWRYMRLAG | |
| | | | QVFFLALIAWGWRCIRHGGLGTYTG | |
| | | | LILVWAGPFLLMLWSLAYQFILALP | |
| | | | VTNTALPIFLPTLYLWVVDTLALRR | |
| | | | GTWVISTGTKYGLHLWDGLEIEEAL | |
| | | | FFLATNALIVFGQLAFDNALAVLYT | |
| | | | FPHLFTGPSLLPSPVLLMRALLTPC | |
| | | | SKYHDARIKGLDEAVNRLKRKSRSF | |
| | | | YLASATFPGPLRADLLLLYSFCRVA | |
| | | | DDLVDNASDADEARAWIAKMRKFLN | |
| | | | NVYSDKLPQSVVHSQICDDFPPSTQ | |
| | | | SALLQLPATKLSPQPLEDLLHGFEM | |
| | | | DLAFQQGPIIRTMEDLRVYSERVAG | |
| | | | TVAQMCIQLIFYWYPSTLDTEEKNV | |
| | | | IVAAGNSMGVALQYVNIARDIEVDA | |
| | | | QIGRVYLPLNWLSEAGLSYDDVLKK | |
| | | | PNQAQIQTLRKHLLNHAFSVYEKAK | |
| | | | DSIERLPIEARGPIRVAVESYMEIG | |
| | | | RILRSEQYQVKAGRATVPKSRRIMV | |
| | | | AWRTLNSK | |
| SEQ | Ra. | Expanded | MLTYMEVHLYYTLPVLGILFWLLRP | Rhizopus |
| ID | CrtYB_ | Sequences | FDSKEDRFKYQFLACIAFFTASVWD | azygosporus |
| NO: | A0A367JYH9 | | NYIVYHKAWSYCPTCVVAVIGYVPL | |
| 232 | | | EEYMFFIIMTFMTVSFTNLVMRWYL | |
| | | | PSTFIRPQTPLIQTWFVRGVPIIVL | |
| | | | MTIAYKAWKLAVPAKPLFYGACILW | |
| | | | YVCPVLAILWYGSGEYMLQRPLSVI | |
| | | | LSITLPTLYLCWVDKIAIAAGTWHI | |
| | | | SLRTSTGKFVSSDLPLEEFMFFTLI | |
| | | | NTVLVFATCTIDRAQAVAQLENKYP | |
| | | | QNTVDQLKMLLWAYLVPDQMLNPQV | |
| | | | INDLSVTWDILKRASKSFHTASVVF | |
| | | | NYAVRQDLSVLYGFCRATDDLCDDE | |
| | | | SVPGEHRKHQLQATRQFVKTMYAEK | |
| | | | KLTEAHYSQLPATCVPAFRAFATHL | |
| | | | RHIVSEESVYELLDGYRWDLEKKSV | |
| | | | KDEDDLAYYSACVASSVGEMCTRII | |
| | | | AHHGQCSLSSDTIQLARQMGLVLQY | |
| | | | INIARDIVTDSQGLRRCYLPQNWLT | |
| | | | QQEIQNLQLGQPRELSDCRLRQISL | |
| | | | KLVSKAELMMKQAEVGIDDLPYCQG | |
| | | | GLRAACQVYAGIGRCLKMSSGYPNR | |
| | | | AYLKKKERIKIVLKSIYL | |
| SEQ | Rd. | Expanded | MLTYMEIHLYFTLPVVGLLLWILKP | Rhizopus |
| ID | CrtYB_ | Sequences | FHSSQDTFKYKFLLTIAFVTASVWD | delemar |
| NO: | I1BJX8 | | NYIVYHKAWSYCPTCVVVVIGFVPL | |
| 233 | | | EEYMFFIIMTVMTVAFANLMMRWHL | |
| | | | ASIFIKPNTPWIQSFFIRFVPILGL | |
| | | | LTIAYKAWRLTVPGKPLFYGSCILW | |
| | | | YACPVLALLWYGSGEFILRRSSSVL | |
| | | | ISIAIPTLYLCWVDILAIREGVWHI | |
| | | | SLRTSTGYFIVPHLPLEEFLFFTLI | |
| | | | NTVLVFATCTEDRVFIILQLTHRKV | |
| | | | TMMNLIWAFALPDQALDHQLFHDLD | |
| | | | QTQAILKRASQSFHAASAAFPAPIR | |
| | | | QDLIVLYGFCRATDDLCDDDQVPIE | |
| | | | SRLQQLEVIRKFVQQVFSQKPTWLY | |
| | | | IAALPASCQPTFRAFATHLAGILKE | |
| | | | ETVLELLDGYQWDLGCQAIRDEADL | |
| | | | QYYSACVASSVGEMCTRVILYHEHA | |
| | | | KQSSWLLDRARQMGLVLQYVNIARD | |
| | | | IVTDSAQLGRCYLPQDWLTSDEIDQ | |
| | | | IAQGQARRLGEDRLKDLSIRLLSKA | |
| | | | DQLMKQAQRGLDGLPASSQGGVRAA | |
| | | | CSVYASIGTVLRQSKIYPTRAHLSS | |
| | | | RQRAITVEKSIYL | |
| SEQ | Rm. | Expanded | MLTYMEVHLYYTLPVLGILLWLLRP | Rhizopus |
| ID | CrtYB_ | Sequences | FDSKEDRFKYQFLACIAFFTASLWD | microsporus |
| NO: | A0A2G4SFV8 | | NYIVYHKAWSYCPTCVVAVIGYVPL | ATCC 52813 |
| 234 | | | EEYMFFIIMTEMTISFTNLVMRWHL | |
| | | | PSTFIRPQTPLIQTWSVRGVPIMAL | |
| | | | MIIAYKAWKLAVPAKPLFYGACILW | |
| | | | YVCPVLAILWYGSGEYMLQRPLSVI | |
| | | | LSITLPTLYLCWVDKIAIAAGTWHI | |
| | | | SLRTSTGKFVSSDLPLEEFMFFVLI | |
| | | | NTVLVFATCTIDRAQAVTQLENKHR | |
| | | | HNTLDQLKTLLWAYLVPDQMLNPQV | |
| | | | INDLSITWGILKSASKSFHTASAVF | |
| | | | NYAVRQDLSVLYGFCRATDDLCDNE | |
| | | | SVPGKRRERQLQATRQFVKTLYAEK | |
| | | | QLTEAHYSQLPVTCIPAYRAFATHL | |
| | | | RYIVSEESVYELLDGYRWDLERRSV | |
| | | | KDEDDLAYYSACVASSVGEMCTRII | |
| | | | AHHGQCSLSSDTIHRARQMGLVLQY | |
| | | | INIARDIVTDSQDLHRCYLPQNWLT | |
| | | | QQEIENLQLGQPRELSDRRLRQISL | |
| | | | KLVSKAELMMKQAEAGIDDLPYCQG | |
| | | | GLRAACQVYAGIGQCLKLSSGYPDR | |
| | | | AYLEKRERIKIVLKSIYL | |
| SEQ | Sm. | Expanded | MYDYAFVHLKFTIPLAVLLTAIAYP | Sordaria |
| ID | CrtYB_ | Sequences | VLNRIHVIQTGCLIFIAFTAALPWD | macrospora |
| NO: | D1Z4K7 | | AYLIEQKVWSYPPEAIVGPRLLGIP | |
| 235 | | | FEELFFFVIQTYITALVYILENKPV | |
| | | | LHALHLNNQRNPPAWMRIAKVTGQL | |
| | | | ILVALSVWGWKAAQVNQKTTYLGLI | |
| | | | LVWACPFLLAIWTLAGRFILSLPWF | |
| | | | VTVLPVVLPTFYLWAVDELALHRGT | |
| | | | WSIGSGTKLEYCLFGKLDIEEATFF | |
| | | | LVTNMLIVSGMAVFDQYLAVIYAFP | |
| | | | TLFPKVNRYPTPLMLVQSRLVNTTK | |
| | | | YDLERIEGLREAVERLRLKSRSFYL | |
| | | | ANSLFSGRLRIDLILLYSFCRLADD | |
| | | | LVDDAKSRLEVLSWTAKLNHELDQH | |
| | | | YGDSDATEDPKQKAERIDAYIKEAF | |
| | | | PPFAYQALHLLPTHILPPKPLYELI | |
| | | | KGFEMDSQFTFHGSSDSTNLKFPIA | |
| | | | HDKDLKTYAIRVAGTVGELCIALII | |
| | | | HHCLPDMSDSQKRRLESAACRMGIA | |
| | | | LQYVNIARDILVDAQIGRVYLPTSW | |
| | | | LKEEGLTHKAVLDNPEGPEVIEKMR | |
| | | | RRLLDNAFELYREARPEMQQIPSEA | |
| | | | RGPMIGAVENYMEIGRVLREKKVGQ | |
| | | | VFVRKEGRATVPKQRRLRTLLRALY | |
| | | | EQ | |
| SEQ | Sp. | Expanded | MGFDYWLVHAKWTIPPALVLWLFFR | Sporidiobolus |
| ID | CrtYB_ | Sequences | RLRTARDVYKTWFLITIAVLATIPW | pararoseus |
| NO: | A0A0K0QV93 | | DSYLIRNRIWSYPQSSVVGPTLFAI | |
| 236 | | | PYEEVFFFFIQTYITSAVYALFTRP | |
| | | | VVHAVLLPTKPEQGRTKRYFGTAIF | |
| | | | LLVTALSITKIKEGGEGTYLALIVG | |
| | | | WVAPFLALLWFITSTHILAMPRYSV | |
| | | | ILPILLPTLYLWECDARALQRGTWV | |
| | | | IEQGTKLGLSYRGLEIEEAVFFLLT | |
| | | | NLMIVFGLVACDYCLAVHDLRSYDK | |
| | | | GYSSPFPPIQDFLPILANQPDTKQA | |
| | | | QRIIDLRNAIDILEVHSKSFSTASM | |
| | | | VFDGRLRLDLLSLYAWCRVCDDLID | |
| | | | NASSVPVASANITGIRTELTYAYTR | |
| | | | YDSTDQDSSTLPRPLTKVQLDTSLP | |
| | | | LLSEKERGPFRLLTFLPITRGPLEE | |
| | | | LLTGFETDLSFIASALEEKEKKKNN | |
| | | | DLPIKTDQDLMDYSSNVASSVADLC | |
| | | | VQLVFAHSTPSSSSASSTSSDESKK | |
| | | | RETLLAARKMGKALQLVNIARDVPA | |
| | | | DQKINRMYLPSRPIDSPIETLTEER | |
| | | | RRLLKLAKEMAEESRVAIEGLPQEA | |
| | | | RGGIRAACDVYLSIGKAVEEALKDG | |
| | | | RIEERARVGKFTRAKTAWRAL | |
| SEQ | Sr. | Expanded | MLTYMEVHLYYTLPMTGLLFLLLRP | Syncephalastrum |
| ID | CrtYB_ | Sequences | FHTAQDRLKYTFLCTIAMLTASPWD | racemosum |
| NO: | A0A1X2H2H5 | | NYIVYHRAWWYCPSCVTAVIGYVPL | |
| 237 | | | EEYMFFVIMSLMTVAFTNLAMRWRL | |
| | | | PSVFVKPDTSVAQSMCTRYVPIAFL | |
| | | | LTIGVRAWNLAIPDTQLFYGACILW | |
| | | | YVCPVLALLWFGAGEYMCRRWQTVL | |
| | | | ISILVPTAYLCWVDKVAIGAGTWHI | |
| | | | SLRTSTGKMVLPNLPLEEAMFFTLI | |
| | | | NTVLVFATCAIDRAQAILHLTKSSR | |
| | | | LRSASQQQKQKESVVQQILDLTWAF | |
| | | | CLSDQGLDNQQLKDIDVTWRILREA | |
| | | | SFSFYTASAVFPMNVRQDLGILYGF | |
| | | | CRATDDLADDEQVSVEKRKDHLALA | |
| | | | RQFVQAMFRDKPNQPVMIDWEDFSN | |
| | | | LPTCCLAAFRAFTRLRPALEVDAVE | |
| | | | ELLDGYAWDLQRRPILDDEALVRYS | |
| | | | ACVASSVGEMCTRVILYNHDSNIAD | |
| | | | TAVSRWTLERARDMGLVLQFTNIAR | |
| | | | DIVTDSQQLRRCYVPRTWLCAEEHD | |
| | | | AICQGKARQHLGDGRLRELALQLVQ | |
| | | | VASDLEKRGRRGIHRLPSECQGGVR | |
| | | | AACAVYSAIGGALQKAKGYPTRAHV | |
| | | | SQAARAWIVLKNVYGFGQTTGIHP | |
| SEQ | Te. | Expanded | MLTYMEVHLYFTLPVIGLLFYVLKP | Thamnidium |
| ID | CrtYB_ | Sequences | FHSKQDTLKYQFLTLMAVSTASVWD | elegans |
| NO: | A0A8H7SM09 | | NYIVYHKAWYYCPTCVVAVIGYVPL | |
| 238 | | | EEYMFFVIQTLMTVAFANLVMRWHL | |
| | | | PTFYIGVNVSISQSLVVKFVPIFGL | |
| | | | LVVAYKAWYAAVPGKPLFYGSCILW | |
| | | | YVCPVLALLWYGAGEYMLRRPLAVL | |
| | | | TSIIVPTIYLCWVDQYAIGAGTWDI | |
| | | | SLKTSTGKMVVTNLPVEECLFFTMI | |
| | | | NIVLVFATCAIDRAQAIVHLYKSQS | |
| | | | IHQRKQDQPFLQQMSDLVWAFCLPD | |
| | | | QMLDNDILKDVAASWDILRTSSKSF | |
| | | | YTASAVFASHDRLDLGILYGFCRAT | |
| | | | DDLCDDESVPVEKRKDQLKVTHQFV | |
| | | | KELFSQKTNTPVIDWEFYAAQLPSS | |
| | | | CVSAFKTFTRLRHILQVDAIEELLD | |
| | | | GYKWDLERRPIVTQQDLYQYSACVA | |
| | | | SSVGEMCTRILLHNNDTGVHTNDRC | |
| | | | ILERAREMGLVLQLTNIARDIVTDS | |
| | | | QELGRCYLPTDWLLKGEDELIATGK | |
| | | | ARQIGEPRLLNLSKKLIQDGERIMV | |
| | | | TANKGIDQLPRNCQGGVRAACNVYA | |
| | | | AIGTQIKQANERYATRAHLKSYGRS | |
| | | | KIALASVYNVHTNTLKKARQGKIRS | |
| | | | FIV | |
| SEQ | Tv. | Expanded | MGLDYLMVHVKYNIPPALLLTILYK | Trichophyton |
| ID | CrtYB_ | Sequences | PFFTRLEVYKIVFLCTIAVVWTTPW | verrucosum |
| NO: | D4D802 | | DSYLIRTRVWSYPADSVIGHTIFRI | |
| 239 | | | PLEEAFFFIIQTYNTSLIYILENKR | |
| | | | LILLPYLSGPIKPLTQGLEGTVALR | |
| | | | TWRDFGILFFTGISVLGISCIRAGG | |
| | | | EYMYLGLILSWISPILLLQWPLMYR | |
| | | | FLLGLPPASLWVPIVLPTLYLWIVD | |
| | | | TLALRRGTWVIESGTKVDIQLWDGL | |
| | | | ELEEALFFLVTNVMIVLGIAGMDNA | |
| | | | IALFEYKAFVSTTAVGETPSIPRLL | |
| | | | TLFFTRSRRYCDTNVLREMSQAVTL | |
| | | | LKQKSQTMYLGSAMFEGQLRLDLVA | |
| | | | LYSFCRKADDLIDDAPNRATAQYWI | |
| | | | KQCEKALELRFKLKGAALDNTAAYQ | |
| | | | QLTKSIPPQLHAAVHLLPASRLPKG | |
| | | | PLSDLLKGFEIDMKFDSERGIFPIA | |
| | | | TEHDLEVYAYHVAGTIATLLLELVF | |
| | | | RHHPVSISDSERLRVISAGEGMGRA | |
| | | | LQYTNIARDIVRDAEIGRVYIPSVW | |
| | | | LAEQGLTPSMVVNQPRNPKLIPLRR | |
| | | | RLLDKAEKCYRDTQEAISELPANVR | |
| | | | APVRATVTVYMDIGQVIRENEMKVW | |
| | | | NGKLKVSRWRREKGAWLAMS | |
| SEQ | Um. | Expanded | MTSLDEPSQDWPACTLSYRHFHLLW | Ustilago maydis |
| ID | CrtYB_ | Sequences | TLPVCGVLLVIARPFLTKLDRAKLI | |
| NO: | Q4P0S6 | | LLPIIAFVWTTPWDNLIVRNRAWSY | |
| 240 | | | HRHCIWFTIGYVPIEEYFYFIIQSL | |
| | | | ISTLWCTLITRWTLPNLYLTPSTPA | |
| | | | RRPLAPFAVATCVVCFVLGLQASVP | |
| | | | ATHTYYFGMISWWSSMPLALLLWGS | |
| | | | IDFVRNMGVGAGFIPFAISVLGPTL | |
| | | | YLWSSDIYALRRGTWHINQATSLNI | |
| | | | FPIPDLPIEEMLFFLVINVILVSAC | |
| | | | FTFDRCVAICRLSAPDHQPPLSPSY | |
| | | | LPLGSLATYTKLWAAFVRSDRSSLP | |
| | | | TSCASASVEPQDLSASLQVLRAASK | |
| | | | SFNAASLLLPWDLRTDLGCLYAFCR | |
| | | | VADDLVDDHAQELQAKAKNLDVVRD | |
| | | | IVDAVYADSIVDRKQHLSTPIGERL | |
| | | | RILLASAALSDKIKQDTRAAATSIA | |
| | | | PLTQYIPKRLWYEMLQGYSADLRFQ | |
| | | | HQDARQRTRLQSMDDLVEYSQCVAG | |
| | | | VVGEMCIRVILGRCGSTVPLDLEVD | |
| | | | RKIALRASKTIEQSINVKPLDLNRT | |
| | | | ADVHTLLYEARRMGVSLQLVNIARD | |
| | | | VVPDSVELRRCYLPADMEDKEEDDG | |
| | | | MYEALLKGRVALPRHASKLVHGAEE | |
| | | | QGVVRPQDVRKYALRLLEISQRLYD | |
| | | | ESYPSLRQIPNRPARAGLKAACSVY | |
| | | | AAIGKRIQAQTEDEISSGRRARMSN | |
| | | | LDRILRAISAVYFDV | |
| SEQ | Xd. | | MTALAYYQIHLIYTLPILGLLGLLT | Phaffia |
| ID | CrtYB_ | | SPILTKFDIYKISILVFIAFSATTP | rhodozyma |
| NO: | mutant | | WGSWIIRNGAWTYPSAESGQGVFGT | (Xanthophyllomyces |
| 241 | D52G | | FLDVPYEEYAFFVIQTVITGLVYVL | dendrorhous) |
| | | | ATRHLLPSLALPKTRSSALSLALKA | |
| | | | LIPLPIIYLFTAHPSPSPDPLVTDH | |
| | | | YFYMRALSLLITPPTMLLAALSGEY | |
| | | | AFDWKSGRAKSTIAAIMIPTVYLIW | |
| | | | VDYVAVGQDSWSINDEKIVGWRLGG | |
| | | | VLPIEEAMFFLLTNLMIVLGLSACD | |
| | | | HTQALYLLHGRTIYGNKKMPSSFPL | |
| | | | ITPPVLSLFFSSRPYSSQPKRDLEL | |
| | | | AVKLLEEKSRSFFVASAGFPSEVRE | |
| | | | RLVGLYAFCRVTDDLIDSPEVSSNP | |
| | | | HATIDMVSDFLTLLFGPPLHPSQPD | |
| | | | KILSSPLLPPSHPSRPTGMYPLPPP | |
| | | | PSLSPAELVQFLTERVPVQYHFAFR | |
| | | | LLAKLQGLIPRYPLDELLRGYTTDL | |
| | | | IFPLSTEAVQARKTPIETTADLLDY | |
| | | | GLCVAGSVAELLVYVSWASAPSQVP | |
| | | | ATIEEREAVLVASREMGTALQLVNI | |
| | | | ARDIKGDATEGRFYLPLSFFGLRDE | |
| | | | SKLAIPTDWTEPRPQDEDKLLSLSP | |
| | | | SSTLPSSNASESFRFEWKTYSLPLV | |
| | | | AYAEDLAKHSYKGIDRLPTEVQAGM | |
| | | | RAACASYLLIGREIKVVWKGDVGER | |
| | | | RTVAGWRRVRKVLSVVMSGWEGQ | |
| SEQ | Xd. | | MTALAYYQIHLIYTLPILGLLGLLT | Phaffia |
| ID | CrtYB_ | | SPILTKEDIYKISILVFIAFSATTP | rhodozyma |
| NO: | mutant | | WDSWIIRNGAWTYPSAESGQGVFGT | (Xanthophyllomyces |
| 242 | E83K | | FLDVPYEKYAFFVIQTVITGLVYVL | dendrorhous) |
| | | | ATRHLLPSLALPKTRSSALSLALKA | |
| | | | LIPLPIIYLFTAHPSPSPDPLVTDH | |
| | | | YFYMRALSLLITPPTMLLAALSGEY | |
| | | | AFDWKSGRAKSTIAAIMIPTVYLIW | |
| | | | VDYVAVGQDSWSINDEKIVGWRLGG | |
| | | | VLPIEEAMFFLLTNLMIVLGLSACD | |
| | | | HTQALYLLHGRTIYGNKKMPSSFPL | |
| | | | ITPPVLSLFFSSRPYSSQPKRDLEL | |
| | | | AVKLLEEKSRSFFVASAGFPSEVRE | |
| | | | RLVGLYAFCRVTDDLIDSPEVSSNP | |
| | | | HATIDMVSDFLTLLFGPPLHPSQPD | |
| | | | KILSSPLLPPSHPSRPTGMYPLPPP | |
| | | | PSLSPAELVQFLTERVPVQYHFAFR | |
| | | | LLAKLQGLIPRYPLDELLRGYTTDL | |
| | | | IFPLSTEAVQARKTPIETTADLLDY | |
| | | | GLCVAGSVAELLVYVSWASAPSQVP | |
| | | | ATIEEREAVLVASREMGTALQLVNI | |
| | | | ARDIKGDATEGRFYLPLSFFGLRDE | |
| | | | SKLAIPTDWTEPRPQDEDKLLSLSP | |
| | | | SSTLPSSNASESFRFEWKTYSLPLV | |
| | | | AYAEDLAKHSYKGIDRLPTEVQAGM | |
| | | | RAACASYLLIGREIKVVWKGDVGER | |
| | | | RTVAGWRRVRKVLSVVMSGWEGQ | |
| SEQ | Bv. | | MQVDAFDTSAPDLLLVGGGLANGLL | Brevundimonas |
| ID | CrtY_ | | ALRLSQVRPDLDVRIVEAAQTLGGV | vesicularis |
| NO: | Q0H2D0 | | HTWSFFDSDLTQAQRDWIAPLVVHR | DC263 |
| 243 | | | WPGYSVRFPQFERALSTPYCSVTAE | |
| | | | RFAAVVEAALPGRIMLGAPVASVSP | |
| | | | TEAVLADGRRLSAKAVIDGRGPTAT | |
| | | | PDLALGFQKFVGLEVRLAAPHGLTT | |
| | | | PIVMDACVDQAGGYRFLYTLPFDDR | |
| | | | TLLIEDTRYTDGDALDRAAFRQGVL | |
| | | | DYAHAQGWEIETILREEDGVLPVAL | |
| | | | DGDIGAHLSRMGLTALSGLRAGLFH | |
| | | | PTTGYSLPDAVRLADRLAQDFEPAT | |
| | | | VAEDIRRYAHDVWAGRGFYRLLNRM | |
| | | | LFRAARPDERYKVLERFYRLPQPLV | |
| | | | ERFYAAGSTLADKARILSGKPPVPI | |
| | | | GAALTCLVERGRA | |
| SEQ | Psa. | | MTGAQDADLLIAGAGLSAGLIALAV | Paracoccus |
| ID | CrtY_ | | RAARPDCRVLLVDAAPAPTGSHTWS | sanguinis |
| NO: | A0A1H3ANX2 | | FHLPDLPPAWLARLSPGLRARWDGQ | |
| 244 | | | EVRESDHARRLRAGYAALDDAGLAR | |
| | | | LVADSGAEVIRAAPVAQADAAGLVL | |
| | | | ADGRRLSAPCVIDARGARPSRHLVT | |
| | | | GFQKFVGLEIETEAPHGVARPVIMD | |
| | | | ATVPQEDGYRFVYLLPFSPTRLLIE | |
| | | | DTRYSDGAALDDAALADAIAAYARA | |
| | | | RGWAGREVRRERGVLPIALAHDAAG | |
| | | | FWDEAAGGAVPVGLRAGLFHPVTGY | |
| | | | SLPVAARVADAVAAASPLTTAAAMA | |
| | | | AVRRLALTKAREDRFLRLLNRMLER | |
| | | | GCPPERRHRILSRFYRLPEGLIERF | |
| | | | YAGRLTTLDRLRIVTGKPPIPIRDA | |
| | | | LPCLPEAPLFKESP | |
| SEQ | El. | | MSDSEIDSVPNDDSCDCAIVGGGLA | Erythrobacter |
| ID | CrtY_ | | GGLIALALQRARPEFRIRVIEAGRT | longus OCH101 |
| NO: | O06756 | | IGGNHRWSWFDSDLSDAGRALLADE | |
| 245 | | | RQTDWEGGYEVRFPKYRRKLKTAYR | |
| | | | SMASTDFHEGLLRALPEGSVILGRK | |
| | | | AVGLDARGVDLAPSQYGPATRINAR | |
| | | | SVIDCRSFKPSAHLKGGWQVFLGRH | |
| | | | MRLQEPHGVENPVIMDATVDQLAPH | |
| | | | GNGGSYRFVYVLPLGSHDVFIEDTY | |
| | | | YADDPLLDRNALSGRIDQYARANGW | |
| | | | ENGTPVHHEAGVLPVLTGGDFSAYQ | |
| | | | DEVRIPGVAIAGARGGFTHPLTSYT | |
| | | | MCVAVENALAMAEQPDLSGEQLAAF | |
| | | | FDSRARRHWSKTGYYRLLARFLFFA | |
| | | | AKPEKRVKVFQRFYGLREGLIERFY | |
| | | | AARSNTFDKVRVLWGEPPVAIHSAI | |
| | | | LAMFKSGPALKSEKSDRGVAQAALD | |
| | | | EELQTEKRP | |
| SEQ | Rsp. | | MESGSHDLSLPIVLVGAGLASAVIA | Rhizobium sp. |
| ID | CrtY_ | | QRLSTVRDAPPIVILEATDTPFGEH | Leaf321 |
| NO: | WP_ | | TWSFHKADLQPSSLAWIAPLIAHEW | |
| 246 | 62583193.1 | | DGQSVRFGSYERHLPSGYASLTSTS | |
| | | | VSEAIERLPNVSLSKNTEVTALSAA | |
| | | | QVTFANGSTQAASCVIDCRGYRPSA | |
| | | | SMVLGYQKFVGLEVELSEPHGLPHP | |
| | | | VIMDATVDQLDGYRFVYLLPMSPTR | |
| | | | VLIEDTRYSEGADLNDAVISEDIHT | |
| | | | YATRQGWDITQVVRRENGVLPIVLA | |
| | | | HDFERFWSELPADIPHAGLRAGLFH | |
| | | | PTTGYSLPDAVHVADLIGENWPIGS | |
| | | | AALPRAIRGHAARQHKKQGFYRLLN | |
| | | | RMLFLAAEPDRRHLVLERFYKLPTP | |
| | | | LIERFYAGRTTMTDILRILTGKPPV | |
| | | | PIHRALACLSEAKLFENGSS | |
| SEQ | Ds. | | MERPLVCAGAGLASALIAARLTRLH | Devosia soli |
| ID | CrtY_ | | PEQKIILLEGSSEPFGEHTWSFHLA | |
| NO: | A0A0F5L7L4 | | DVNEADLEWLAPLIAHRWTGQAVRE | |
| 247 | | | QKLRRHLGSGYASLTSASVASAIAA | |
| | | | MANVEVVAGAKVRDLAPESVTLADG | |
| | | | RSIAAAAVIDTRGYTPSDALVLGYQ | |
| | | | KFVGLEVELEEPHGLSDPVIMDASV | |
| | | | DQRDGYRFIYLLPFSPTRVLIEDTR | |
| | | | YSDGEALDLQALAGDIARYAEAQGW | |
| | | | SIRQVVREENGVLPIALAHDFERFW | |
| | | | ADKPVDIPQAGMRAGLFHPTTGYSL | |
| | | | PEAVRIANLVAGNWPASSVTLARAI | |
| | | | RDHARMRHREQGFYRLLNRMLFRAA | |
| | | | APERRHLVLQRFYKLPQPVIERFYA | |
| | | | GRSTLGDIARILIGKPPVPIQRALA | |
| | | | CLREAPLLKAKKA | |
| SEQ | Cv. | | MESASEALRADVVLVGGGLANSLIA | Caulobacter |
| ID | CrtY_ | | LRLKALRPGLRVLLLEREATIGGEH | vibrioides |
| NO: | A0A0V2F4L4 | | TWCHFETDVDAAIGGWLRPLIVHRW | |
| 248 | | | TGYEVREPAHSRRLPTPYLAITSRR | |
| | | | LHEVVSRVLGPDAWTGVAVVDVNPH | |
| | | | QVTLADGRRIVAGAVIDGRGPRRSK | |
| | | | RLALGWQKFVGQDLRLVAPHGLTAP | |
| | | | IIMDATVPQTDGYRFVYVLPLDQHR | |
| | | | LLIEDTRYSDGPGLDRASLGAAIAD | |
| | | | YARAQGWTIEAVEREEHGVLPIALG | |
| | | | GDIDAYWREARPQVAEVGLRAALFQ | |
| | | | PTTGYSLPDAARLADKVAALPRITS | |
| | | | ASVRACVVTHSKATWRKRRFLRLLN | |
| | | | RMLFRACAPEERYRVLERFYRLSVP | |
| | | | LIQRFYAARLTWRDKARVLIGKPPV | |
| | | | PIGAAMKCISESSVEGGQDA | |
| SEQ | Psp. | | MTHDVLLAGAGLANGLIALALRAAR | Paracoccus sp. |
| ID | CrtY_ | | PDLRVLLLDRAAGPSDGHTWSCHDP | PAMC 22219 |
| NO: | P54974 | | DLSPEWLARLKPLRRANWPDQEVRE | |
| 249 | | | PRHARRLVTGYGSLDGAALADAVVR | |
| | | | SGATIRWNSDIATLDAQGATLSCGT | |
| | | | RIDAGAVIDGRGAQPSRHLTVGFQK | |
| | | | FVGVEIETDRPHGVPRPMIMDATVT | |
| | | | QQDGYRFIYLLPFSPTRILIEDTRY | |
| | | | SDGGDLDDDALAAASHDYARQQGWT | |
| | | | GVEVRRERGILPIALAHDAAGFWEN | |
| | | | HATGPVPVGLRAGFFHPVTGYSLPY | |
| | | | AAQVADVVAGLPGPLTTDALRAAIR | |
| | | | DFAIARARRDRFLRLLNRMLFRGCA | |
| | | | PDRRYTLLQRFYRMPQGLIERFYAG | |
| | | | RLTVADQLRIVTGKPPIPLGTAIRC | |
| | | | LPERPLLKENA | |
| SEQ | Rb. | | MEHFDFIIIGGGASGLLLAEAMGRD | Robiginitalea |
| ID | CrtY_ | | TWFDGKRIALFEKQEQKGNDRTWCF | biformata |
| NO: | A4CHD3 | | WESGEGAFDELIETRWDRMRFRGTG | HTCC2501 |
| 250 | | | IDRVLPLLPLTYKMLRGEDFYASYY | |
| | | | QKIATCPNIHVIREAVNHVEERPDG | |
| | | | AFLKTSRASYTTSRVFSSVPFSQPP | |
| | | | LPGRPYPLIRQHFIGWRVRVPRPVF | |
| | | | DPDTPTFMDFGIPQRGNTREMYVLP | |
| | | | DSPTEALLEYTLFSKDLLEPGEYEA | |
| | | | AIEAYLREEYGVGEYEILEKERGSI | |
| | | | PMTCNDFTARDSKHITHIGIAGGWA | |
| | | | RPSTGYTFWNSTQLVPRLVKALKSG | |
| | | | KRLQMARRDRFWFYDLVLLEVLAGN | |
| | | | NARGGEVESALFRRVPPTRILRFLH | |
| | | | QQTSLGEDLRIIAACPKRLFIRALW | |
| | | | RSFLKSLGGAATPQNRAAPPWPGGF | |
| | | | R | |
| SEQ | As. | | MNGVERHDRPIDVAFVGGGLANGLA | Aurantimonas |
| ID | CrtY_ | | AYRLARARPELSIRVFEAGEAVGGN | sp. SI85-9A1 |
| NO: | Q1YGX9 | | HTWSFHAGDLSAAEHDWLAPFVRYR | |
| 251 | | | WDAYDVRFPQRRRQIATGYRSVSSE | |
| | | | QFRAVIDAALGDRIERHAPVRAVTP | |
| | | | TTLTLEDGRELAAVCVIDGRGHAPS | |
| | | | PHLMLGFQKFLGQEIELAAPHGLSR | |
| | | | PIIMDATVAQEDGYRFVYVLPLTAT | |
| | | | TLLVEDTYYADGPALSEERLRTTIA | |
| | | | DYLATHGWTAKAVLREERGVLPIAL | |
| | | | DGDIEAFWADRRGVPATGLAAALFH | |
| | | | PTTGYSLPDAVRLADRLAGLTDLSA | |
| | | | GAVFAATRAHSVAAWRERGFFRLLN | |
| | | | RLLFFAGVPSKRYEILQHFHRLPDT | |
| | | | LIGRFYAAQLSRLDMVRIMTGRPPV | |
| | | | PVGNALKVLARHRLTGELA | |
| SEQ | Pz. | | MSHDLLIAGAGLSGALIALAVRDRR | Paracoccus |
| ID | CrtY_ | | PDARIVMLDARSGPSDQHTWSCHDT | zeaxanthinifaciens |
| NO: | P94791 | | DLSPEWLARLSPIRRGEWTDQEVAF | R1534 |
| 252 | | | PDHSRRLTTGYGSIEAGALIGLLQG | |
| | | | VDLRWNTHVATLDDTGATLTDGSRI | |
| | | | EAACVIDARGAVETPHLTVGFQKFV | |
| | | | GVEIETDAPHGVERPMIMDATVPQM | |
| | | | DGYRFIYLLPFSPTRILIEDTRYSD | |
| | | | GGDLDDGALAQASLDYAARRGWTGQ | |
| | | | EMRRERGILPIALAHDAIGFWRDHA | |
| | | | QGAVPVGLGAGLFHPVTGYSLPYAA | |
| | | | QVADAIAARDLTTASARRAVRGWAI | |
| | | | DRADRDRFLRLLNRMLFRGCPPDRR | |
| | | | YRLLQRFYRLPQPLIERFYAGRLTL | |
| | | | ADRLRIVTGRPPIPLSQAVRCLPER | |
| | | | PLLQERA | |
| SEQ | De. | | MPIPGQGPLVLVGAGLASALIAQRL | Devosia enhydra |
| ID | CrtY_ | | AQSFPDQPILMLEAAAAPFGEHTWS | |
| NO: | A0A1K2HSZ0 | | FHEPDISRADWQWVGPLVAHRWPGQ | |
| 253 | | | QVRFVAHARKLSTGYGSLTSQSVAD | |
| | | | AMARLANLTIRTLAPVRDIRPDGVT | |
| | | | LSSGEAIPAACVIDASGHRQSPHML | |
| | | | LGFQKFVGLEIETTEPHGVDVPMIM | |
| | | | DASVDQKDGYRFVYLLPFSPTRILI | |
| | | | EDTRYADGEALDMQALAADISAYAE | |
| | | | AKGWTLRTVVREEHGVLPIALAFDA | |
| | | | ERFWADAPKDIPQAGMRAGLFHPTT | |
| | | | GYSLPEAVRIANLVAESWPAGSAAL | |
| | | | AERIRRHALKRAKDQSFYRFLNRML | |
| | | | FKAAVPDRRHLVLQRFYRLPQPLVE | |
| | | | RFYAGRTHWGDIARILIGKPPVPVS | |
| | | | RALPCLLEAPQLNRSNKKTVTS | |
| SEQ | Pa. | | MRPHYDLILVGAGLANGLIALRLQQ | Pantoea |
| ID | CrtY_ | | QQPDMRILLIEAAPQAGGNHTWSFH | agglomerans |
| NO: | Q47844 | | DADLTESQHRWVAPLVVYHWPDYQV | Eho10 |
| 254 | | | RFPTRRRKLNSGYFSVTSQRFAEVL | |
| | | | QQKFGQHLWISRAVAEVHADAVRLN | |
| | | | NGQVISASAVIDGRGYTPNSALNVG | |
| | | | FQAFIGQEWRLSKPHGLSSPIIMDA | |
| | | | TVDQQNGYRFVYSLPLSATELLIED | |
| | | | THYIDNATLEPERARQNIRDYAAQQ | |
| | | | DWQLQTLLREEQGALPITLTGDSTA | |
| | | | FWQQQPLACSGLRAGLFHPTTGYSL | |
| | | | PLAVALADRLSALDVFTSSSIHQAI | |
| | | | THFAHERWQQQRFFRMLNRMLFLAG | |
| | | | PADSRWRVMQRFYGLPEDLISRFYA | |
| | | | GKLTLTDRLRILSGKPPVPVLAALQ | |
| | | | AIMTTHR | |
| SEQ | Eb. | | MRTQYDVILVGAGLANGLIALRLRQ | Enterobacteriaceae |
| ID | CrtY_ | | LQPQLKCLLLESDAHPAGNHTWSFH | Bacterium |
| NO: | Q2VJ84 | | HSDLSAEQLRWLQPLITVRWSGYQV | DC260 |
| 255 | | | RFPALRRNLDGDYCSIASGDFARHL | |
| | | | YAAMGDDLWTNTAVQQVKPTQVTLA | |
| | | | DGRELAAQVVIDGRGLQPTPHLQLG | |
| | | | YQVFLGQEWQLAQPHGLQQPILMDA | |
| | | | TVDQQAGYRFVYTLPLSADRLLIED | |
| | | | THYVNQPALAENTARQHIADYANQQ | |
| | | | GWTLSTLLREEHGILPITLSGNIDR | |
| | | | FWQQQRGQACSGLRAGLFHATTGYS | |
| | | | LPSAVALAELVAALLPTDALTLSQH | |
| | | | IERFARQQWREQRFFRLLNRMLFLA | |
| | | | GKPQQRWRVMQRFYRLDAGLISRFY | |
| | | | AGQLRLRDKTRILCGKPPVPIGEAL | |
| | | | RALLNSVEPGKKK | |
| SEQ | Tf. | | MERVDVAIVGAGAAGLSLAHRLDRV | Thermobifida |
| ID | CrtY_ | | LPHRSVVLVETPEAALRSPERTWCF | fusca YX |
| NO: | Q47KA1 | | WEEETGEWDAAVCHRWHALAVQGPD | |
| 256 | | | GTTWRSRIHPLVYKMIRSRDFEALV | |
| | | | RGSLQRVRTLHWTVTDIVDGPGEAV | |
| | | | LRLRGADGAQRELAAGLVFDSRPLV | |
| | | | GLPAEGVTLLQHFRGWFVRTDRPRF | |
| | | | DSSAAVLMDLRVPQPERGVAFGYLL | |
| | | | PFDARTALVEYTEFTREVCSDAAYD | |
| | | | AALRDYTENLLHLGVFTVTGVEQGV | |
| | | | IPMTSAHLPERVGHRVFRIGTAGGA | |
| | | | TRPATGYTFAGVQRQTRRIAAALVA | |
| | | | GRFPVGYSAYPARHRFFDAVLLRGL | |
| | | | DEGVVDGAEFFATLFRSNPLPRLLR | |
| | | | FLDGSSRPWEELAIGLGTPVADMST | |
| | | | ALWRQVRAGRRVASSLPR | |
| SEQ | Pb. | | MTSYDVLFAGAGLASSLTAYRLARR | Parvularcula |
| ID | CrtY_ | | TPSLHIGMVEQGAAVGGNHTWCFHE | bermudensis |
| NO: | E0THV9 | | TDIAPAILEGLKPFILKRWPGQSVR | HTCC2503 |
| 257 | | | FPGHDRDLPTPYCAMTSQRLAAVLS | |
| | | | GVPSIDVITGAQVTKATADRLILAD | |
| | | | GRELSAPCVFDGRGPGRVRHMRLGF | |
| | | | QKFLGLEVTLAAPHGLNQPIIMDAT | |
| | | | VPQTDGYRFVYVLPFSETELLIEDT | |
| | | | YYADGFDLSESHLEAEIIHYAARKG | |
| | | | WTIATISRREQGVLPIVLEGDFEAF | |
| | | | WAERRTGAVPIGLAAGLFHPVTGYS | |
| | | | VPDAATLADRLGRDTPLTSDRVRPI | |
| | | | VEDYARDRWSAHGFYRLLNRLLFRA | |
| | | | AAPDQRYIVLERFYRLPIPLIERFY | |
| | | | AGETNTGDKLRVLAGKPPVPLMRAI | |
| | | | SALRPQPLGHSAGMREGILG | |
| SEQ | Cs. | | MNTQWDLILAGGGLANGLIALRLRV | Cronobacter |
| ID | CrtY_ | | RQPRLNVLLLEASDAPGGNHTWSFH | sakazakii ATCC |
| NO: | A7MP98 | | GADLTAEQHAWLTPLVAHRWDGYEV | BAA-894 |
| 258 | | | RFPALTRTLDGGYYSVTSERFAHVL | |
| | | | QEAVGERLWRNAPVASLTPQSVTLT | |
| | | | DGRTLHARAVIDGRGWQASSHLTAG | |
| | | | QQAFLGQQWRLAAPHGLTRPVLMDA | |
| | | | TVAQQGGYRFVYTLPLTPDTLLIED | |
| | | | THYIDSATLDHDRARENIASYARLQ | |
| | | | GWQLATLEREERGNLPITLTGAPQA | |
| | | | FWREAQGVARAGLRAGLFHATTGYS | |
| | | | LPHAATLADLIAAQPPVSSAALYGL | |
| | | | TAGYAQRQWRRQRFFRLLNRMLFLA | |
| | | | GQPDRRWQVMQRFYGLNAGLIARFY | |
| | | | AGRLTPMDMARLLTGKPPVPVGEAM | |
| | | | QAVLKQTPRLRAFHHD | |
| SEQ | CPe. | | MPNATYDLLLAGAGLANALIVRALK | Candidatus |
| ID | CrtY_ | | ASHPDLKIGIVDRNPHPDPSHTWSF | Phaeomarinobacter |
| NO: | X5MCP7 | | HTSDISDDARAWLDPLIAHEWHGQD | ectocarpi |
| 259 | | | VRFPGHARALKTGYASISEEKLRHQ | |
| | | | LAQDMEATPILEGDVTSLDEEGVTL | |
| | | | EDGRHIDAHAVIDGRGQKANTSLAI | |
| | | | RFQKFVGQELELEEPHVLTRPVIMD | |
| | | | ATVPQEDGYRFFYLLPHTPTSLLVE | |
| | | | DTRYSDGAELNDDVERDAIENYATG | |
| | | | KGWKIKKVLREERGVLPIALGGNFD | |
| | | | AFWEGTPKGTGRSGLAAALFHPVTG | |
| | | | YSLPDNVRLAQAIAALPHITSQTVA | |
| | | | QTTRAHAKAAWDKRGFYRLLNRMLF | |
| | | | DAARPQDRYKVLERFYRLPEPLIAR | |
| | | | FYAGQTTLADKARILTGKPPVPILA | |
| | | | AMRCLKEPKSGVSQP | |
| SEQ | Asp. | | MPERFDLVLVGGGLANGLIAHRLAV | Aureimonas |
| ID | CrtY_ | | LRPDIGVLVLEAEAAPGGNHTWSFH | |
| NO: | A0A0Q6E1S9 | | AGDLSEAEAAWLAPFVVHRWPAYDV | |
| 260 | | | RFPRRLRRLGTGYASVTSERFAEVL | |
| | | | QAGRPGLVRCGMRVAELRPDGVTLS | |
| | | | DGTQIAAAAVIDGRGPRPSARLSIG | |
| | | | YQKFLGRELRLSAAHGLSAPVVMDA | |
| | | | TVAQDDGYRFVYTLPFDERRLLVED | |
| | | | TYYADGPGLDGADLALRIAAYVAER | |
| | | | GWAAAEVLREEEGILPIALGGDIEG | |
| | | | FWADRAGVAASGLSAALFHPTTGYS | |
| | | | LPDAVRLADLVAGLPDLSAPALFAA | |
| | | | TRDHSIRLWRERRFFRLLNRMLFLA | |
| | | | GASERRYEILQHFHRLPEDLIARFY | |
| | | | AARLSLGDKARILAGKPPVPVGRAL | |
| | | | RALAFPRSLSPEDAA | |
| SEQ | Xa. | | MDIVFVGAGLANCLMAARLAAQRPG | Xanthobacter |
| ID | CrtY_ | | LHMLLLEAGESVGGNHSWSCHDSDL | autotrophicus |
| NO: | Q93CI8 | | TAAQRAFLAPFQSYLWAGHGVHFPA | Py2 |
| 261 | | | FSRTLKGGYATISSERMAEVMNERL | |
| | | | CAAIRTNARVAHVAPDHVVLEGGER | |
| | | | IDARAVVDGRGPLASRHLDLGYQTF | |
| | | | LGQELRMSRPHGLTRPIIMDARVEQ | |
| | | | LGGYRFVYVLPLDDDTLLVEDTYYA | |
| | | | DGPDLPADALRGRISAYAAAQGWAV | |
| | | | DYVVREEDGILPIALGGDINAFLAE | |
| | | | TPSGVAPAGLRAGLFHPTTGYSLPD | |
| | | | AMALADSVSALADLSGPALSAAVRS | |
| | | | HAAAAWNGRGFFRLLNRMLFRAADP | |
| | | | ERRYAILQRFYGLSEDLIARFYADR | |
| | | | LTLADKARILSGRPPVSVFRALSCL | |
| | | | VETKAAPGSP | |
| SEQ | Psph. | | MSGVLIVGAGLAGSLAALALRAARP | Paracoccus |
| ID | CrtY_ | | GLPVTLLDAAPGPSDRHTWSFHHPD | sphaerophysae |
| NO: | A0A099F1A2 | | LPPVWRDRLAPGLRASWSLQEVRFP | |
| 262 | | | DHGRVLEAGYASLDGRGLAQLVAGS | |
| | | | GATTLWSRTAAEVLAHAVTLSGGTR | |
| | | | LTADAVLDARGLRPSRHLVAGYQKE | |
| | | | VGVEYEMDAPHGVICPLIMDAKVPQ | |
| | | | LDGYRFVYLLPFSPTRLLIEDTRYS | |
| | | | DGAGLAEPDLIAAITDYAAVHGWQG | |
| | | | REVRRERGVLPIALAHDAAGFWAAQ | |
| | | | DGAVPIGLRAGLFHPVTGYSLPVAV | |
| | | | QVADVIAAADPTTAAIRAAVRDFAL | |
| | | | RKAHGDRFLRLLNRMLFRGCAPDER | |
| | | | RRVLSRFYRLPEPLIERFYAGRLTT | |
| | | | LDKLRIVTGKPPIPIRDALPCLPES | |
| | | | PLLKDAR | |
| SEQ | Bs. | | MQAAAPDSSAPDLLLLGGGLANGLL | Brevundimonas |
| ID | CrtY_ | | ALRLSQVRPELDVRIVEAADRLGGI | sp. SD212 |
| No: | Q4W8B7 | | HTWSFFEADLTPAQRAWIAPLIACR | |
| 263 | | | WPGYSVRFPAFERRLVTGYCSVTAE | |
| | | | RFAEAVTQALAGRIVTGAAVVSAGP | |
| | | | TEAVLADGHRLTARAVIDGRGPTAA | |
| | | | PDLALGFQKFVGLEVRLTAPHGLKE | |
| | | | PIVMDACVDQSGGYRFLYVLPFDDR | |
| | | | TLLIEDTRYTDGDDLDHDLFRTGVR | |
| | | | DYAAQRGWVIETVLREEEGVLPVAL | |
| | | | DGDIAAHLKRLGPTALSGLRAGLFH | |
| | | | PTTGYSLPDAVRLADHLAERIEAAP | |
| | | | DGPALAQVIRRHARDVWAQRGFYRL | |
| | | | LNRMLFRAARPDQRYRVLERFYRLP | |
| | | | QPLIERFYAGETTLADKARILSGKP | |
| | | | PVPIGAALTCLVERGRA | |
| SEQ | Ch. | | MATQSSNTRADVVLVGGGLANGLIA | Caulobacter |
| ID | CrtY_ | | LRLRTLRPDLRVILLEREKRIGGEH | henricii |
| NO: | A0A0P0P0Q7 | | TWCHFGTDVSPGISGWLRPLIVHRW | |
| 264 | | | SDYEVRFPGHSRALKTPYLAITSQR | |
| | | | LHEAVSAALGQDAWLSATASEVSPH | |
| | | | QVTLTDGRRITAAAVVDGRGPRRSR | |
| | | | SLALGWQKFLGQEVKLTSPHGLTRP | |
| | | | IIMDATVSQLDGYRFLYVLPLDASR | |
| | | | LLIEDTRYSDGFDLDRDALRADILA | |
| | | | YANQQGWTVEKIVREEHGVLPIALA | |
| | | | GDIDAYWREARAQVAEVGLRAALFQ | |
| | | | PTTGYSLPDAARLADQIAALPHITS | |
| | | | AHVRACAEAQSKTAWRKRRYFRLLN | |
| | | | RMLFRACEPDQRFKVLERFYRLRLP | |
| | | | LIQRFYAARLTLWDKARVLIGKPPV | |
| | | | PVSRALNYLSEHSVIEDRPA | |
| SEQ | Pa. | | MQPHYDLILVGAGLANGLIALRLQQ | Pantoea |
| ID | CrtY_ | | QQPDMRILLIDAAPQAGGNHTWSFH | ananatis ATCC |
| NO: | P21687 | | HDDLTESQHRWIAPLVVHHWPDYQV | 19321 |
| 265 | | | RFPTRRRKLNSGYFCITSQRFAEVL | |
| | | | QRQFGPHLWMDTAVAEVNAESVRLK | |
| | | | KGQVIGARAVIDGRGYAANSALSVG | |
| | | | FQAFIGQEWRLSHPHGLSSPIIMDA | |
| | | | TVDQQNGYRFVYSLPLSPTRLLIED | |
| | | | THYIDNATLDPECARQNICDYAAQQ | |
| | | | GWQLQTLLREEQGALPITLSGNADA | |
| | | | FWQQRPLACSGLRAGLFHPTTGYSL | |
| | | | PLAVAVADRLSALDVFTSASIHHAI | |
| | | | THFARERWQQQGFFRMLNRMLFLAG | |
| | | | PADSRWRVMQRFYGLPEDLIARFYA | |
| | | | GKLTLTDRLRILSGKPPVPVLAALQ | |
| | | | AIMTTHR | |
| SEQ | Bo. | | MSRDADVIVIGGGLAGCLIALRLTD | Bradyrhizobium |
| ID | CrtY_ | | ARPDLRVVIIEGSASIAGNHTWSFF | sp. ORS278 |
| NO: | Q9KIX3 | | GTDISSDQHAWLGRLVGHRWPGYEV | |
| 266 | | | RFAEHAIRLSTAYLSMTSTRLRAEV | |
| | | | EQRFPERILRDATAISATADHVVLE | |
| | | | GGRTLRAPCVIDARGGRPVPGLALG | |
| | | | FQKFLGLEVRLAAPHGLDVPIVMDA | |
| | | | TVAQSDGYRFVYTLPLDPQRLLIED | |
| | | | TYYSDGGELPEQVLHQRIARYALAK | |
| | | | GWQIAEIIRAEQGVLPVILAGDPSG | |
| | | | LVSKPDSPPRVGLAALLVHPTTGYS | |
| | | | LPDAVRVADLLTARLAQRGALSSAD | |
| | | | ARETIDGYGRTIWRRRGYYRFLNRM | |
| | | | LFKAAEPSERHRILARFYGLDQALI | |
| | | | ERFYAARIQPQDKLRVFMHMLMKPP | |
| | | | IPISSALACLPEASAFKTP | |
| SEQ | Umv49E08. | | MIYLEFLILFIGIPLFAISFWAYKK | Uncultured |
| ID | CrtY_ | | QCLDKKNITGIGLMCVIALVYTTPW | Marine |
| NO: | A4GIB9 | | DNYLIMENIWSYPAGVVFGTIGYVP | Bacterium |
| 267 | | | IEEYGFMLMQTMLAGVLWSLVSSKI | HF10_49E08 |
| | | | NVSGLTLSGKGILVSLIPGFVGSYC | |
| | | | LSHETGTYAGLILVWAFPPLALQWG | |
| | | | LGARALLTSAKMWMPLWVGFTLYLC | |
| | | | LADAFAISDGIWTITSNTRSGIEIM | |
| | | | GILPIEEALFFALTNLFVLQGLCLW | |
| | | | QTWRNKG | |
| SEQ | Umb29C11. | | MKTQILGDGVAAMMLASHADELPNH | Uncultured |
| ID | CrtY_ | | EISIVHPTGAPITRDHMLGFWKMDG | Marine |
| NO: | A4GI69 | | LEMAVESSRTSWSKWAIITDGNKSV | Bacterium |
| 268 | | | MHSDKHAYHIMHKANYLHNCREKAE | HF10_29C11 |
| | | | QHGVKFIEESEMITSKSAQTFDSRP | |
| | | | PRASKNAMLQHFLGQEVEVDKPVED | |
| | | | PSTAILMDFRVDQSEGMHFIYLLPY | |
| | | | SPTQALVESTLFTTTVLERQFYVDA | |
| | | | INEHLRDHYGASITNIIHEEQGVIP | |
| | | | MGTLSPHDDTIPGIGANAGAIRPAS | |
| | | | GYTFVFIHQQIQRAIKASKQGKPLR | |
| | | | FKRPHKAVDVWMDAVLLTVLKNWPQ | |
| | | | QGPKLFGRMASSLSGDEFVREMSGQ | |
| | | | SNWRLRFKVIMAMPKLPFIRGVSKH | |
| | | | IFRRSEKVVA | |
| SEQ | Sk. | | MAEPVDVLVLGGGPAALCVASELNQ | Synechococcus |
| ID | CrtY_ | | RGVSVAGIASDPVDAPWPNTYGIWA | sp. KORDI-52 |
| NO: | A0A076HH15 | | DELKAVGLEQLLEHRWSDTVSFFGE | |
| 269 | | | GGSTAQDRSHAHGIDYGLFDRAVLQ | |
| | | | RHWLERADGVVWHQDTAERVELKGS | |
| | | | TTLVSCASGTTMQARLVIDASGSRT | |
| | | | HHIRRPDQGPVAGQAAYGVVGRFSK | |
| | | | PPIEPDRFVLMDYRCDHLSETQRQE | |
| | | | PPTFLYAMDLGDGVFFVEETSLALA | |
| | | | PAVPYDVLQQRLQQRLDQRGVEITE | |
| | | | VIHEEFCLFPMNLPLPDRRQPLLAF | |
| | | | GGAASMVHPASGYMVGSLLRRGPDL | |
| | | | AQALAEAITNPSLGSAALAQRGWQA | |
| | | | LWPLELVLRHQLYQFGLGRLMGENE | |
| | | | ALLRTHFATFFALPRDEWEGELTNT | |
| | | | LPLPRLMSVMLRLFALAPWELRRGL | |
| | | | VLGAPPSQSPTWTQSNG | |
| SEQ | Sm. | | MVDPLAPETASTGKTGTAAIAGRDL | Sinorhizobium |
| ID | CrtY_ | | ARAGDRERLESESIDPSLPVVLVGA | meliloti |
| NO: | WP_ | | GLASAIIAQRLSTVRDAPQIVILEG | |
| 270 | 50578984.1 | | TDTPFGENTWSFHKADLQPSSLGWI | |
| | | | AALIAHQWDGQSVRFGGYERHLPSG | |
| | | | YASLTSASVRDAIERMPNVTLNKNT | |
| | | | EVTALSTAQVTFANGSTQAASCVID | |
| | | | CRGYRPSSSMVLGYQKFVGLEAELS | |
| | | | EPHGLPHPVIMDATVDQLDGYREVY | |
| | | | LLPLSPTRVLIEDTRYSEGADLDHA | |
| | | | VISDDIHAYATRQGWDITQVVRTED | |
| | | | GVLPIVLAHDFERFWSELPEDIAHA | |
| | | | GLRAGLFHPTTGYSLPDAVHVAEII | |
| | | | AENWPICSAALAHAIRGHAARQHKK | |
| | | | QGFYRLLNRMLFLAAQPDRRHLVLE | |
| | | | RFYNLPTPLIERFYAGRTTTTDILR | |
| | | | ILTGKPPVPIHRALACLNEAHLFEN | |
| | | | GSS | |
| SEQ | Eh. | | MPRYDLILVGAGLANGLIALRLRQQ | Enterobacter |
| ID | CrtY_ | | RPSLRILLIDAEREPGANHTWSFHA | agglomerans |
| NO: | L0BIU3 | | EDLTETQHRWIAPLVVHHWPGYEVR | |
| 271 | | | FPQRSRSLNSGYFCVTSERFVQVIR | |
| | | | DRFAPDLLLNTRVAGIASRTVTLDD | |
| | | | GRVLESDAVIDGRGYQPDAALCMGF | |
| | | | QSFVGQEWQLSEPHGLTAPIIMDAT | |
| | | | VDQQAGYRFVYSLPFSADTLLIEDT | |
| | | | HYIDNATLEGDRARQNIRAYAAQQG | |
| | | | WRLDRLLREEQGALPITLTGDVAAF | |
| | | | WQKHDLPCSGLRAGLFHPTTGYSLP | |
| | | | LAVALADRLAQMQTFTSETLHATIQ | |
| | | | QFASQAWQQQRFFRMLNRMLFLAGP | |
| | | | ADQRWQVMQRFYGLPEGLIARFYAG | |
| | | | KLTLPDRLRILSGKPPVPVLAALQA | |
| | | | IMTPHRQQAMQ | |
| SEQ | Ak. | | MGNYLYLAVNLFAISFPLIRSFEPK | Algoriphagus |
| ID | CrtY_ | | INFARKWYALFPALVITAIFFLVWD | sp. KK10020C |
| NO: | Q15I92 | | HWFTVIGIWEFNPNYLLGIYLFKLP | |
| 272 | | | IEEWLFFFTVPFACVFIYEVLIYFF | |
| | | | PKDIFRPVGRPFVLVLIPILLLLAF | |
| | | | LNLDKLYTSVNFFVGAFALAIHWLI | |
| | | | FKDKFLGRFIFAYLVHLIPFIICNG | |
| | | | ILTGGLTEEPVVIYNNAENLGIRIW | |
| | | | TVPIEDTIYSMTLLLMNVSLFEYFR | |
| | | | SRKTIQQTKSY | |
| SEQ | Pa. | | MPANSHYDVILVGAGLANGLIALRL | Pantoea sp. |
| ID | CrtY_ | | RQQHPSLRCLLLESSAQADSNHTWS | (strain At-9b) |
| NO: | E6WK12 | | FHQGDLRAEQHRWLAPLVTAHWPGY | |
| 273 | | | QVRFPALQRNLPDAYFSISSANFAR | |
| | | | QLYATLGDDLRTGCAVHLITPTSVT | |
| | | | LTDGRQFQAQVVIDGRGMRYSPHLR | |
| | | | LAYQVFVGQEWQLAKPHGLTQPLLM | |
| | | | DATVPQQQGYRFVYTLPLSADRLLI | |
| | | | EDTHYMDTPLLDDNETRKRISDYAR | |
| | | | QQGWQPSLLMREERGALPIPLSGDI | |
| | | | NAFWQQQRGQPCSGLRAGLFHATTG | |
| | | | YSLPMAVTLAELIAERLPCDAAVLS | |
| | | | QHIEAHARRHWHQQRFFRLLNRMLF | |
| | | | LAAQPEQRWQVMQRFYQLDAALIAR | |
| | | | FYAGQLTLADKARILCGKPPVPPGA | |
| | | | ALRALLTTQTGKHP | |
| SEQ | Eu. | | MTHDVLLAGAGLANGLIALALRAAR | Erwinia |
| ID | CrtY_ | | PDLRVLLLDHAAGPSDGHTWSCHDP | uredovora |
| NO: | P54974 | | DLSPDWLARLKPLRRANWPDQEVRF | |
| 274 | | | PRHARRLATGYGSLDGAALADAVVR | |
| | | | SGAEIRWDSDIALLDAQGATLSCGT | |
| | | | RIEAGAVLDGRGAQPSRHLTVGFQK | |
| | | | FVGVEIETDRPHGVPRPMIMDATVT | |
| | | | QQDGYRFIYLLPFSPTRILIEDTRY | |
| | | | SDGGDLDDDALAAASHDYARQQGWT | |
| | | | GAEVRRERGILPIALAHDAAGFWAD | |
| | | | HAAGPVPVGLRAGFFHPVTGYSLPY | |
| | | | AAQVADVVAGLSGPPGTDALRGAIR | |
| | | | DYAIDRARRDRFLRLLNRMLFRGCA | |
| | | | PDRRYTLLQRFYRMPHGLIERFYAG | |
| | | | RLSVADQLRIVIGKPPIPLGTAIRC | |
| | | | LPERPLLKENA | |
| SEQ | Ca. | | MDTLLRTPNNLEFLHGFGVKVSAFS | Capsicum annuum |
| ID | CrtY_ | | SVKSQKFGAKKFCEGLGSRSVCVKA | |
| NO: | Q43415 | | SSSALLELVPETKKENLDFELPMYD | |
| 275 | | | PSKGVVVDLAVVGGGPAGLAVAQQV | |
| | | | SEAGLSVCSIDPNPKLIWPNNYGVW | |
| | | | VDEFEAMDLLDCLDATWSGAAVYID | |
| | | | DKTTKDLNRPYGRVNRKQLKSKMMQ | |
| | | | KCILNGVKFHQAKVIKVIHEESKSM | |
| | | | LICNDGITIQATVVLDATGESRSLV | |
| | | | QYDKPYNPGYQVAYGILAEVEEHPF | |
| | | | DVNKMVFMDWRDSHLKNNVELKERN | |
| | | | SRIPTFLYAMPFSSNRIFLEETSLV | |
| | | | ARPGLGMDDIQERMVARLSHLGIKV | |
| | | | KSIEEDEHCVIPMGGPLPVLPQRVV | |
| | | | GIGGTAGMVHPSTGYMVARTLAAAP | |
| | | | VVANAIIQYLSSERSHSGDELSAAV | |
| | | | WKDLWPIERRRQREFFCFGMDILLK | |
| | | | LDLPATRRFFDAFFDLEPRYWHGEL | |
| | | | SSRLFLPELIVFGLSLFSHASNTSR | |
| | | | LEIMTKGTLPLVHMINNLLQDKE | |
| SEQ | Cm. | | MFDALVIGSGPAGLAIAAELAQRGL | Cellulophaga |
| ID | CrtY_ | | KVQGLSPVDPFHPWENTYGIWGPEL | sp. MED134 |
| NO: | Q55276 | | DSLGLEHLFGHRWSNCVSYFGEAPV | |
| 276 | | | QHQYNYGLFDRAQLQQHWLRQCEQG | |
| | | | GLQWQLGKAAAIAHDSHHSCVTTAA | |
| | | | GQELQARLVVDTTGHQAAFIQRPHS | |
| | | | DAIAYQAAYGIIGQFSQPPIEPHQF | |
| | | | VLMDYRSDHLSPEERQLPPTFLYAM | |
| | | | DLGNDVYFVEETSLAACPAIPYDRL | |
| | | | KQRLYQRLATRGVTVQVIQHEEYCL | |
| | | | FPMNLPLPDLTQSVVGFGGAASMVH | |
| | | | PASGYMVGALLRRAPDLANAIAAGL | |
| | | | NASSSLTTAELATQAWRGLWPTEKI | |
| | | | RKHYIYQFGLEKLMRFSEAQLNHHF | |
| | | | QTFFGLPKEQWYGFLTNTLSLPELI | |
| | | | QAMLRLFAQAPNDVRWGLMEQQGRE | |
| | | | LQLFWQAIAAR | |
| SEQ | Cp. | | MSLAYELDLHNKLKDATLAIIEPRL | Candidatus |
| ID | CrtY_ | | EYKRDKTWSYWKTIDHNFQDCVQKS | Pelagibacter |
| NO: | Q4FPE3 | | WNNFSINIPGKTKFLECNGLPYEAI | ubique HTCC1062 |
| 277 | | | DSGLFYEKINTRLKKNKNISFYKNT | |
| | | | NEIDLNSAFIFNSVPLLDSKEDKLW | |
| | | | QHFKGIEIETKKNVFDDEIFNLMDF | |
| | | | DCDQRDSVHFFYTLPYTKNKALIET | |
| | | | TWLSKIKNDSLKDYDEQLKNYIRNH | |
| | | | LNIRDYKVTYSEVGAIPLFHPTISN | |
| | | | ERNKMNIGTAGGMTRLSTGYTFLNI | |
| | | | QEHSKYIRKNIHTIKESKRYEINKK | |
| | | | YKFLDKIFLKVLNYHPEKMANIFFD | |
| | | | MFNNSPETIIKFLSNKSTLLQDLFI | |
| | | | ILKMPKWLFIKIFFKNDK | |
| SEQ | Cp. | | MIADKVQTLYPRVSEYFGKIRNGNR | Chlorobium |
| ID | CrtY_ | | YLEHILEVDSFWKNFEHSPCRKVVL | phaeobacteroides |
| NO: | A1BCY2 | | QGSEMPADACVSDEYDLVYAGGSLG | DSM 266 |
| 278 | | | LLHASVMASKYARKVLVFDRHMSGK | |
| | | | PSRDWNISWNELEKLEQVGLFSYEE | |
| | | | INASIACRYKTGWAEFYVENGRKKR | |
| | | | LYIDNVLDCAVESDQLLSIARQKIL | |
| | | | ADECNRIVDKMTFMRCFQFPESVVI | |
| | | | EVENNMHERVYFRAKVFVDAMGVHS | |
| | | | PVAMQLNDGASFTHLCPTVGTVSSG | |
| | | | LENVDPDIGEILVSTEPADTSSGRG | |
| | | | RQLIWEGFPAGGDRYTTYLFFYDSR | |
| | | | SSDNDKSLLGLFETYFLKLPSYKKP | |
| | | | GKDFVVHRPVYGVIPAYYHDGFERT | |
| | | | RQVADNNILMLGDAAALGSPLTFCG | |
| | | | FGSFVRNLKSLTEGLEKALACNSFE | |
| | | | KKNLEQISAYEPNVAAMANLMKEMC | |
| | | | YNKYTDHPNFVNELMNEVMIVLDGL | |
| | | | PPRYREAMFKDTLELSDLLLIGLNV | |
| | | | AWKYPGVLMATATKLGLEGSLGMMK | |
| | | | NMIGWAMSSKASN | |
| SEQ | Ct. | | MLEQIRNTHPLVYQNFSALPDGEKH | Chlorobaculum |
| ID | CrtY_ | | LRSILAIDRYWEKLDLPVPDVILAG | tepidum TLS |
| NO: | Q8KF75 | | SRLPVDACVEEACDILYAGGTLGLL | |
| 279 | | | HAAVMSKKYGRKVLVIDRAEPGRTT | |
| | | | RDWNISRGELLRLADTGVFTSEELD | |
| | | | STIVRMYKTGWVEFHAPAERRKRLY | |
| | | | MDEVLDCAVDADRLLGMACKKVLAG | |
| | | | GGSKVLGHTSFVCCYQFPDHLVVQV | |
| | | | EELSGKPRYFRTQVLVDAMGIVSPV | |
| | | | AMQLNRGRPQTHVCPTVGTIASGFE | |
| | | | NADFEVGEILASTEDAEVSGKRGRQ | |
| | | | LIWEGFPAKGDEYITYLFFYDKVDS | |
| | | | PNDKSLLGLFEAYFRKLPEYKKPGP | |
| | | | NFTIHRPVFGIIPAYFHDGAGCTRV | |
| | | | VSGERIALLGDAASLASPLTFCGFG | |
| | | | SVVRNLDRMTSGLDRAMREGRLGAA | |
| | | | ELANISAYEPNVASMATLMKYMCYD | |
| | | | PETDEPGFVNEMMNEVMIVLDELPQ | |
| | | | RYRQAMFRDEMKVEELVTVMLKVAW | |
| | | | RYPKILKATWNKLGVGGSVGFVKNL | |
| | | | AGWAISQNEKRG | |
| SEQ | Fp. | | MTSSAKQKVDIALVGGGLANGLIAW | Fulvimarina |
| ID | CrtY_ | | RLAELRPDLSIVVLEAGEAPGGNHT | pelagi HTCC2506 |
| NO: | Q0G4C0 | | WSFHEHDLTPAAHRWIAPFVAHRWT | |
| 280 | | | TNEVQFPDRHRHLSTGYLSASSDLF | |
| | | | RERLTTRLGLRIRTGCPAVSVTARK | |
| | | | VRLENGEVIEAGSVIDGRGYRSSEH | |
| | | | LTLGFQKFLGQEIEFEAPHGVARPV | |
| | | | IMDATVPQADGYRFVYLLPMTPTRL | |
| | | | LVEDTYYADGDALDRGTIRRNIAAY | |
| | | | RAAKGWPAGKVVREEDGVLPIALAG | |
| | | | DIEAFWEEKQGVPSSGLNAALFHPT | |
| | | | TGYSLPDAVYLADLIAGLPDYSAAT | |
| | | | LYAATRRHSVATWKRRGFFRMLNRL | |
| | | | LYLAGDPLKRYVILQHFYRLPEPLV | |
| | | | SRFYAARLTRGDKVRILTGKPPVSV | |
| | | | ISALKVLSPSSVEGAPA | |
| SEQ | H1. | | MLPTLTYLQFHLVESLPVIAALWYV | Halorubrum |
| ID | CrtY_ | | APRYGAVRQRRATIAIAILVAIAYT | lacusprofundi |
| NO: | B9LUN5 | | YTTPWISYMIRRGAWSYADGAVLVR | ATCC 49239 |
| 281 | | | ALSIPLGEYLFFTVQTVVTAFALHR | |
| | | | IGFDPTFREGDFDRGPRIAGVVAGV | |
| | | | AMILGGLWLVTIGPSYLYLGGLIAW | |
| | | | VGPVVAIQWAVGGGYLIRTPRTWIA | |
| | | | ATLIPAAYFWIADRIAIEMRTWQLS | |
| | | | PDLTTGVAVLGLPIEELLFFVSAGV | |
| | | | MTVNGLVLFEWVLDWNDRRERPTES | |
| | | | LADALEPEPEPESAEPVDD | |
| SEQ | O1. | | MRRGDATATATATAAFAKRREGASD | Ostreococcus |
| ID | CrtY_ | | ARGETYDVAVAGGTLGVLVAAALQR | lucimarinus |
| NO: | A4RQU2 | | RGARVVVVERGELRGREQEWNVSRE | CCE9901 |
| 282 | | | ELERLVRVGAITTEDADACTRIEEN | |
| | | | PIRCGFHGGGNGNGNAGDLVTRDIL | |
| | | | NTGVSPAALVEACRKRFEEAGGAVL | |
| | | | ERASLRGVEVYDDCAVLDVDGVPVR | |
| | | | ARLVLDCMGFQSPIVRQIRDDRKPD | |
| | | | GVCVVVGSCAEADAFDNSSADLIRT | |
| | | | VTDIEEDYRGQYFWEAFPASTGPRD | |
| | | | RTTYMFTYMDADESRPSIASMLDDY | |
| | | | WDLMPLYQGLDSIEDAKLKRVLFGL | |
| | | | FPTYRDSPLKTEFDRVLAIGDASGI | |
| | | | QSPLSFGGLAAILRHISRITGACEE | |
| | | | ALDADCLDREALRTVNAYQPALSAA | |
| | | | WLFQKCMSVEVGSRPKRDFINRLMR | |
| | | | INFNVMSRLGEDVLRPFLQDVVTFK | |
| | | | GLGKTLVSMTLTEPLFVPEILMNAG | |
| | | | VGPILDWFKHFAALGAYDELASPAA | |
| | | | ALADTVKDFSAVSPRRRFIIRRQCE | |
| | | | AIIYGAGRDVIP | |
| SEQ | Ph. | | MTHDVLLAGAGLANGLIALALRAAR | Paracoccus |
| ID | CrtY_ | | PDLRVLLLDHAAGPSDGHTWSCHDP | haeundaensis |
| NO: | Q24K62 | | DLSPHWLARLKPLRRANWPDQEVRF | |
| 283 | | | PRHARRLATGYGSLDGAALADAVAR | |
| | | | SGAEIRWNSDIALLDEQGATLSCGT | |
| | | | RIEAGAVLDGRGAQPSRHLTVGFQK | |
| | | | FVGVEIETDCPHGVPRPMIMDATVT | |
| | | | QQDGYRFIYLLPFSPTRILIEDTRY | |
| | | | SDGGNLDDDALAAASHDYARQQGWT | |
| | | | GAEVRRERGILPIALAHDAAGFWAD | |
| | | | HAEGPVPVGLRAGFFHPVTGYSLPY | |
| | | | AAQVADVVAGLSGPPGTDALRGAIR | |
| | | | DYAIDRARRDRFLRLLNRMLFRGCA | |
| | | | PDRRYTLLQRFYRMPHGLIERFYAG | |
| | | | RLSVADQLRIVIGKPPIPLGTAIRC | |
| | | | LPERPLLKENA | |
| SEQ | Po. | | MADAYDLLFAGGGLSSGLTAYRVAQ | Parvularcula |
| ID | CrtY_ | | ARSDLSIAIVEAAPRLGGNHTWSFH | oceani |
| NO: | WP_ | | ATDVAPGTLRWLTPFIVHDWPAQRV | |
| 284 | 31553302.1 | | AFPRRERRIETPYRSVTSERLHEVV | |
| | | | SAVPNISLLTGKPVKAVRRDGLTLE | |
| | | | DGAVLSGPVLDGRGARSSPHLSLGF | |
| | | | QKFLGQELRTARPHGVETPLIMDAR | |
| | | | VPQTDGYRFVYVLPLGPDILHVEDT | |
| | | | YYADGDDLPREALREEIMAYAHRQG | |
| | | | WEVAEILREEDGVLPIVLEGDIDGF | |
| | | | WDAHRAGPAPLGLRAGLFHPVTGYS | |
| | | | LPSAARLADEIAEACGSAIPAPGAL | |
| | | | FELVEDHARRQWEAQGFYRLLNRML | |
| | | | FRAAKPDLRYVVLQRFYGLPRALIE | |
| | | | RFYAGETTAADKARILAGKPPVPLG | |
| | | | AALRSLRPQALLGQG | |
| SEQ | Se. | | MSGSALRSVLNQLPARVAQGLHQSD | Synechococcus |
| ID | CrtY_ | | AFWRSLRWGQLPTPQWVSESADPLG | elongatus PCC |
| NO: | Q31QI5 | | SCDLDVLVAGGTLGLLPALGLQRQG | 7942j |
| 285 | | | WRVGLWERGMVQGRDQEWNISRHEL | |
| | | | AVLVELELLTAEELEAAIATEFPAA | |
| | | | RIAFPGSAELWVEGILNIGISPRRL | |
| | | | IATLLQHFREQGGQVFEQTAIDRVS | |
| | | | VHPDGVVIGAGSQRWSGRLLLDALG | |
| | | | HFSPIARQARAGAKPDAICLVVGGC | |
| | | | AQGFPASDRGDLFVSRSPIQQQRQS | |
| | | | FWEAFPAAEGRTAYLFTYIDAQPAR | |
| | | | PSLRSLLEEYLAALPEYQGVDLQQL | |
| | | | QWKRLLWGILPSYRQSPLQSPWNRI | |
| | | | LAIGDSSSAQSPLSFGGFGALLRHL | |
| | | | ERLCRGIHEALLADCLQAADLALLQ | |
| | | | PHQPNLAVTWLFQQSMTVPLDRSLP | |
| | | | PERINELLGRIFAVMESLGPQTLRP | |
| | | | FLQDVVQFQPLAKTLLMTTLLHPVL | |
| | | | VAKLLPQLGLQPLLQWLPQFLQLSL | |
| | | | YRSLAIAGEPTDWLKVCDRDRFRQR | |
| | | | RRQEAWFYGSGCDYAAPLKSSVDRH | |
| | | | RSIN | |
| SEQ | Sg. | | MPTDFDVVIVGAGAAGMSLAYHLCA | Streptomyces |
| ID | CrtY_ | | PDSDVPLSVALVDAPPGPLRAPPRT | griseus NCBI |
| NO: | P72451 | | WCFWEPPGGPYDPVLAASWPRLRVR | 3933 |
| 286 | | | AADGASTVAQLPRLRYKMLRSDAFE | |
| | | | ALVEQRFSRAPDLCRMEATASSVRD | |
| | | | DPSGVGGEVLTRTACGERILVRGRL | |
| | | | VFDSRPSHRLPPARTTLLQHFTGWE | |
| | | | VRTERPVFDPGTADLMDFRTPQPAR | |
| | | | GLSFGYVLPLDPHTALVEYTEFSPA | |
| | | | PLDTDGYRRALRHYTHDVLRLGPLQ | |
| | | | VTAQEHGVIPMTDGRFPHKAGRSVY | |
| | | | RIGTAGGATRPSTGYTFAAVQRQSR | |
| | | | AVADQLRSGRPLRVPAPYGRRARLM | |
| | | | DAVLLRALDSGRVDGADFFHRLFRH | |
| | | | IPGERLLSFMDGRSQLHEDLLIGLR | |
| | | | TPMVPMLRTVFELPERTRRARPAAP | |
| | | | FPPHRPPPKGAPHDPVAR | |
| SEQ | Ss. | | MEIFKPDYLMIDSMIFFPTLILSFL | Sulfolobus |
| ID | CrtY_ | | IKRNYVKLLKSIGIVSPIYLFWDFL | solfataricus P2 |
| NO: | Q97UT9 | | ATWRNSWTENPKYVAGIYVIDLPIE | |
| 287 | | | EVMFFLVTPFATLLIFDFVMGKVRD | |
| | | | KEVKWVPKVVCVAIPLLLLTLPFVY | |
| | | | NYSYTFIDLVYLIMSLIVSLLIGRN | |
| | | | LLASRNFWIFMGLSYIPFLVFDYFL | |
| | | | TSDPVVIYGSHSIVGIRFITIPIED | |
| | | | FIYSFSMITFYTVFYIRGNSLWMKQ | |
| | | | K | |
| SEQ | Va. | | MKVNVAILGAGCAGLALARCLKEQG | Vibrio angustum |
| ID | CrtY_ | | FSGSVLLLESRQCYENDRTWCYWAK | S14 |
| NO: | Q1ZMY2 | | PDSQWHKMAQYRWQQCLFSSLNDAQ | |
| 288 | | | HIHYYDSTSYCCLPAENYYDFCLQQ | |
| | | | LDSANVSLKMEVEVVSVSPTRSQSW | |
| | | | LVETTAGNLEADIVIDTRPRPCEAI | |
| | | | LYQSFLGYEVTLSRPIFAPDTVRLM | |
| | | | CNMHGDNQCMRFYYILPFSPWHILI | |
| | | | EPTVFHRYPLNPDLLKDDLQSALRA | |
| | | | EDIEIESIIRRESAILPMGQTHLEG | |
| | | | SVLVKGGIAGGALRASSGYGELRIQ | |
| | | | AWALACAQSIITKQKAFSHQQHHLQ | |
| | | | ARMDHLFLNVIKHNPVDGPDYFIRM | |
| | | | ASQLNGDQFSAFMSDEAGMKEWMQV | |
| | | | ISALPKWPFIKEVMAGG | |
| | | | N-term | |
| SEQ | Ref Name | Organism | truncation | AA sequence |
| SEQ | Ss.GGPPS_ | Syn- | No | MVVADAHTQGFSLAQYLQEQKTIVE |
| ID | B1XJV9 | echococcus | | TALDQSLVITEPVTIYEAMRYSLLA |
| NO: | | sp. PCC | | GGKRLRPILCLAACEMLGGTAAMAM |
| 289 | | 7002 | | NTACALEMIHTMSLIHDDLPAMDND |
| | | | | DLRRGKPTNHKVYGEDIAILAGDAL |
| | | | | LSYAFEYVARTPDVPAERLLQVIVR |
| | | | | LGQAVGAEGLVGGQVVDLESEGKTD |
| | | | | VAVETLNFIHTHKTGALLEVCVTAG |
| | | | | AILAGAKPEEVQLLSRYAQNIGLAF |
| | | | | QIVDDILDITATAEELGKTAGKDLE |
| | | | | AQKVTYPSLWGIEKSQAEAQKLVAE |
| | | | | AIASLEPYGEKANPLKALAEYIVNR |
| | | | | KN |
| SEQ | Oq.GGPPS_ | Ostreobium | Yes | MLETRPPPSTSETETFDFKGYMKEQ |
| ID | CAD7703201. | quekettii | | AALVETALDSAVPLARPETIHESMR |
| NO: | 1 | | | YSLLAGGKRVRPVLCLAACELVGGS |
| 290 | | | | LENAMPTACALEMLHTMSLIHDDLP |
| | | | | SMDNDDLRRGKPTNHKMFGEDMAIL |
| | | | | AGDALLTFAFEHVACATKGVAADRV |
| | | | | LKVITNLGKAVGTRGLVAGQVVDLE |
| | | | | SEGKVVDLDVLEYIHEHKTAALLEA |
| | | | | AVVCGATLGGASDEEIEKLRKYSRN |
| | | | | IGLAFQVIDDILDVTQTSEHLGKTA |
| | | | | AKDLASDKTTYPKLLGLEKSREVAQ |
| | | | | HLIDEAVAQLASENSTRVAPLVGLA |
| | | | | HYIGSRTN |
| SEQ | Cd.GGPPS_ | Chlamy- | Yes | MATAEQVEVKPGSFDENSYMTERAK |
| ID | KAG2491721. | domonas | | LVNKALDEAVPQKYPDTLYESMRYS |
| NO: | 1 | debaryana | | LLAGGKRVRPALCLAACELVGGDIQ |
| 291 | | | | AALPSACAMEMVHTMSLIHDDLPSM |
| | | | | DNDDFRRGRPTNHKVFGEDIAILSG |
| | | | | DALLTYSFEHIARATKGVPAERVLR |
| | | | | VVMELGKAVGAEGLVAGQVVDIQSE |
| | | | | DKEVGLDVLRYIHTSKTAALLEASV |
| | | | | VCGAILGGADDGTIEKLRKYSLNIG |
| | | | | LAFQVIDDILDVTQTTEQLGKTAAK |
| | | | | DLAVNKTTYPKLLGLEKSKQVAEDL |
| | | | | ITEAIQQLDGFDKARAAPLVALAKY |
| | | | | IGYRQN |
| SEQ | At.GGPPS_ | Arabidopsis | Yes | MSVVTKEDNLRQSEPSSFDEMSYII |
| ID | P34802 | thaliana | | TKAELVNKALDSAVPLREPLKIHEA |
| NO: | | | | MRYSLLAGGKRVRPVLCIAACELVG |
| 292 | | | | GEESTAMPAACAVEMIHTMSLIHDD |
| | | | | LPCMDNDDLRRGKPTNHKVFGEDVA |
| | | | | VLAGDALLSFAFEHLASATSSDVVS |
| | | | | PVRVVRAVGELAKAIGTEGLVAGQV |
| | | | | VDISSEGLDLNDVGLEHLEFIHLHK |
| | | | | TAALLEASAVLGAIVGGGSDDEIER |
| | | | | LRKFARCIGLLFQVVDDILDVTKSS |
| | | | | KELGKTAGKDLIADKLTYPKIMGLE |
| | | | | KSREFAEKLNREARDQLLGFDSDKV |
| | | | | APLLALANYIAYRQN |
| SEQ | Bt.GGPPS_ | Blakeslea | No | MLTSSKSIESFPKNVQPYGKHYQNG |
| ID | H9CDX4 | trispora | | LEPVGKSQEDILLEPFHYLCSNPGK |
| NO: | | | | DVRTKMIEAFNAWLKVPKDDLIVIT |
| 293 | | | | RVIEMLHSASLLIDDVEDDSVLRRG |
| | | | | VPAAHHIYGTPQTINCANYVYFLAL |
| | | | | KEIAKLNKPNMITIYTDELINLHRG |
| | | | | QGMELFWRDTLTCPTEKEFLDMVND |
| | | | | KTGGLLRLAVKLMQEASQSGTDYTG |
| | | | | LVSKIGIHFQVRDDYMNLQSKNYAD |
| | | | | NKGFCEDLTEGKFSFPIIHSIRSDP |
| | | | | SNRQLLNILKQRSSSIELKQFALQL |
| | | | | LENTNTFQYCRDFLRVLEKEAREEI |
| | | | | KLLGGNIMLEKIMDVLSVNE |
| SEQ | Hp.GGPPS_ | Haema- | Yes | MVATAGQVAEVHSAPAFDFEMYMRD |
| ID | QHF16627.1 | tococcus | | RAEMVNKALDAALPSRYPEVLVDSM |
| NO: | | pluvialis | | RYSVLAGGKRVRPALTLAACDLVGG |
| 294 | | | | DMATALPTACAMEMIHTMSLIHDDL |
| | | | | PAMDNDDFRRGRPTNHKVYGEDIAI |
| | | | | LAGDALLSFAFEHIARDTKGVPAEA |
| | | | | VLKVIMELGRAVGAQGLSAGQAVDI |
| | | | | KSEGQEVGLEVLEYIHHHKTAALLE |
| | | | | AAVVCGALVGGADTATVEKLRKYAL |
| | | | | NIGLAFQVIDDILDVTQTTETLGKT |
| | | | | AAKDLAVNKTTYPKLLGLEASRKVA |
| | | | | DDLIREAIAQLDEFEPARKAPMVAL |
| | | | | AHFIGYRKN |
| SEQ | Cc.GGPPS_ | Cistus | Yes | MSVLSGKDTMKGEEENSGFDFQSYM |
| ID | AF492023 | creticus | | DQMADSVNQALESAVSLREPLKIHE |
| NO: | | | | AMRYSLLAGGKRVRPLLCIAACELV |
| 295 | | | | GGDVSVAMPAACAVEMIHTMSLIHD |
| | | | | DLPCMDNDDLRRGKPTNHKAFGEDI |
| | | | | AVLAGDALLSFAFEHVAVSTVGASP |
| | | | | DKIVRAVGELAKAVGKEGLVAGQVV |
| | | | | DITSEGLNDVGLDHLEYIHVHKTAV |
| | | | | LLEAAVVLGAILGGGTDEEVERLRK |
| | | | | FAICIGLLFQVVDDILDVTKSSVEL |
| | | | | GKTAGKDLVADKVTYPKLMGLEKSR |
| | | | | EFAEKLRDDAVEQLRVFDQVKAAPL |
| | | | | IALAHYIAYRQN |
| SEQ | Bn.GGPPS_ | Brassica | Yes | MSSIVRKQDNHMKSSSFDFMSYMAR |
| ID | XP_ | napus | | KVETVNRALDLAVPLQEPLKIHEAM |
| NO: | 013655023.1 | | | RYSLLAGGKRIRPLLCIAACELVGG |
| 296 | | | | EASVAMPAACAVEMIHTMSLIHDDL |
| | | | | PCIDNDDLRRGKPTNHKVFGENVAV |
| | | | | LAGDALLPFAFEHLVTATSPEVSPV |
| | | | | RLVRAVGELAKAVGVEGVAAGQVAD |
| | | | | ISSEGLDSNEVGLEHLEFIHLHKTA |
| | | | | ALLEASAVLGGIVGGGSEDEIEKLR |
| | | | | QYARCVGLLFQVVDDILDVTKSSQE |
| | | | | LGKTAGKDLVADKLTYPKIMGLEKS |
| | | | | REFAEKLCRDAREQLLGFVSDRVAP |
| | | | | LLALANYIANRQN |
| SEQ | Hp.GGPPS_ | Haema- | Yes | MVATAGQVAEVHSAPAFDFEMYMRD |
| ID | QHF16627.1 | tococcus | | RAEMVNKALDAALPSRYPEVLVDSM |
| NO: | | pluvialis | | RYSVLAGGKRVRPALTLAACDLVGG |
| 297 | | | | DMATALPTACAMEMIHTMSLIHDDL |
| | | | | PAMDNDDFRRGRPTNHKVYGEDIAI |
| | | | | LAGDALLSFAFEHIARDTKGVPADA |
| | | | | VLKVIMELGRAVGAQGLSAGQAVDI |
| | | | | KSEGQEVGLEVLEYIHHHKTAALLE |
| | | | | AAVVCGALVGGADTATVEKLRKYAL |
| | | | | NIGLAFQVIDDILDVTQTTETLGKT |
| | | | | AAKDLAVNKTTYPKLLGLEASRKVA |
| | | | | DDLIREAIAQLDEFEPARKAPMVAL |
| | | | | AHFIGYRKN |
| SEQ | Ml.GGPPS_ | Melampsora | No | MKPVPSTNGKVDDKVEHHNLSSSSS |
| ID | QIG55789.1 | larici- | | SSSSSSSSDTKFNISNRYGNFLQRL |
| NO: | | populina | | EALDIWPESNEQILLEPYTYLTNIP |
| 298 | | | | GKEIRSMMIDAFNHWLQVPRPALEI |
| | | | | IKKIVGQLHTASLLMDDVEDDSDLR |
| | | | | RGVPVTHKIYGIPQTINTANYVYFL |
| | | | | AYQELTKLKPCLRSDATTDLWSLVN |
| | | | | DELLQLHRGQGMDLYWRDSLTCPTE |
| | | | | EEYLQMVNNKTGGLFRIAIKLMIAL |
| | | | | SPIPETPDYLPLVNLVGIIFQIRDD |
| | | | | LLNLSSVYTKNKGFCEDLTEGKFSF |
| | | | | PIVHSIRSDSTNHQLMNILRQKPTD |
| | | | | IGTKAFAVSYMKDRTKSLEYTRGVL |
| | | | | ICLEEQAIEEVTRLGGNPALESIFE |
| | | | | LMHVLPSPPATDQN |
| SEQ | Cg.GGPPS_ | Coryne- | No | MKDVSLSSFDAHDLDLDKFPEVVRD |
| ID | WP_ | bacterium | | RLTQFLDAQELTIADIGAPVTDAVA |
| NO: | 011014931.1 | glutamicum | | HLRSFVLNGGKRIRPLYAWAGFLAA |
| 299 | | ATCC | | QGHKNSSEKLESVLDAAASLEFIQA |
| | | 13032 | | CALIHDDIIDSSDTRRGAPTVHRAV |
| | | | | EADHRANNFEGDPEHFGVSVSILAG |
| | | | | DMALVWAEDMLQDSGLSAEALARTR |
| | | | | DAWRGMRTEVIGGQLLDIYLESHAN |
| | | | | ESVELADSVNRFKTAAYTIARPLHL |
| | | | | GASIAGGSPQLIDALLHYGHDIGIA |
| | | | | FQLRDDLLGVFGDPAITGKPAGDDI |
| | | | | REGKRTVLLALALQRADKQSPEAAT |
| | | | | AIRAGVGKVTSPEDIAVITEHIRAT |
| | | | | GAEEEVEQRISQLTESGLAHLDDVD |
| | | | | IPDEVRAQLRALAIRSTERRM |
| SEQ | Am.GGPPS_ | Antirrhinum | Yes | MILTKNPQESSQKTSKDPTFTLDFK |
| ID | AY534687 | majus | | TYMLEKASSVNKALEQAVLLKEPLK |
| NO: | | | | IHESMRYSLLAGGKRVRPMLCIAAC |
| 300 | | | | ELVGGLESTAMPSACAVEMIHTMSL |
| | | | | IHDDLPCMDNDDLRRGKPTNHKIYG |
| | | | | EDVAVLAGDALLAFSFEHVAKSTKG |
| | | | | VSSDRIVRVIGELAKCIGSEGLVAG |
| | | | | QVVDISSEGMTEVGLEHLEFIHVHK |
| | | | | TAALLEASVVLGAIVGGADDEDVEK |
| | | | | LRKFARCIGLLFQVVDDILDVTKSS |
| | | | | QELGKTAGKDLVADKTTYPKLLGIE |
| | | | | KSREFAEKLNREAQEQLEGFDSVKA |
| | | | | APLIALANYIAYRDN |
| SEQ | Ca.GGPPS_ | Candida | No | MQLDINKLIQPTEAYVPESSDPIVQ |
| ID | XP_ | albicans | | PYTYISETPSNNQNVRTRFLHAEND |
| NO: | 711426.1 | | | LFYKISDESLLARISEIISVFHNSS |
| 301 | | | | LLIDDIEDDSKYRRGVPVAHVKYGI |
| | | | | PTTINCGNLMYFVALQQAQQLSGPH |
| | | | | GSMEVKFKSSQILIDELLNLHRGQG |
| | | | | LDIYWRDYLKKLEKLPDVEEYLEMI |
| | | | | KDKTGGLFRLAIKLLSLYSDVKEND |
| | | | | QLISLANLMGILYQVRDDYLNLVDA |
| | | | | KYSAMKGTTCEDLIEGKLSLPILHC |
| | | | | LRTTKDSPVHKILYDYDSSSDRVSQ |
| | | | | NSLIDLSLSFMKNESKSLEYTLNLI |
| | | | | KVLEKKLRQLILKYPESENSALLKI |
| | | | | VERLCDL |
| SEQ | Mb.GGPPS_ | Methan- | No | MEADMSDLSAYLKSVAQQIDGMIEK |
| ID | WP_ | oregula | | NFTHAGGELDRASAHLLSAGGKRLR |
| NO: | 012107783.1 | boonei | | PAVVMLSADAIRHGSSKDVMPAALA |
| 302 | | 6A8 | | LEVTHTFTLIHDDIMDGDSLRRGVP |
| | | | | TVHTKWDMPTGILAGDVLYARAFEF |
| | | | | ICQSKADEGPKVQAVALLARACADI |
| | | | | CEGQHQDMSFEHRADVTEEEYMAMV |
| | | | | AKKTGVLYAAAAAIGGTLAGGNPEQ |
| | | | | IRALYQFGLNTGIAFQIQDDLIDLL |
| | | | | TPTEKSGKDQGSDLREGKQTLVMII |
| | | | | ARQKGVDLLKYRHELSPADIKAAIQ |
| | | | | ELTDAGVIDAVKKKAADLVADSNRL |
| | | | | LMVLPPTKERQLIMDVGEFFVTRSF |
| SEQ | Tt.GGPPS_ | Thermus | No | MVPAPEAIRQALQERLLARLDHPDP |
| ID | WP_ | thermo- | | LYRDLLQDYPRRGGKMLRGLLTVYS |
| NO: | 011227641.1 | iphlus | | ALAHGAPLEAGLEAATALELFQNWV |
| 303 | | | | LVHDDIEDGSEERRGRPALHRLHPM |
| | | | | PLALNAGDAMHAEMWGLLAEGLARG |
| | | | | LFPPEVLLEFHEVVRRTAYGQHLDL |
| | | | | LWTLGGTFDLRPEDYFRMVAHKAAY |
| | | | | YTAVAPLRLGALLAGKTPPAAYEEG |
| | | | | GLRLGTAFQIVDDVLNLEGGEAYGK |
| | | | | ERAGDLYEGKRTLILLRFLEEAPPE |
| | | | | ERARALALLALPREAKPEAEVGWLL |
| | | | | ERLLASRALAWAKAEAKRLQAEGLA |
| | | | | LLEAAFQDLPGKEALDHLRGLLAAL |
| | | | | VERRA |
| SEQ | Rs.GGPPS_ | Raphanus | Yes | MEFLDSSTTQAENKEDDTTEFDFKP |
| ID | XP_ | sativus | | YMIRKAESINRALDKAIPLVEPLDI |
| NO: | 018455142.1 | | | HRAMRYALLAGGKRVRPILCLAVCE |
| 304 | | | | LVGGEERLAVPAACAVEMIHTMSLI |
| | | | | KDDLPCMDNDDLRRGKPTTHKVFGE |
| | | | | SVAVLSGGALLALAFEHLTEADVSP |
| | | | | ERMVRAVKELARSIGTKGLVAGQAM |
| | | | | DLSSEGLDRNDVGLEELEFIHVHKT |
| | | | | GSLLEASAVIGAVIGGGSEEEVERV |
| | | | | RKFARCVGLLFQVVDDILDGTKSSE |
| | | | | ELGKTAGKDQVAGKLTYPKVLGLEK |
| | | | | SKEFVERLRRDAREHLKGFDSDKAK |
| | | | | PLVALADFIANRDN |
| SEQ | Al.GGPPS_ | Arabidopsis | Yes | MALTSQGARDMIPPEGKSDDRNSAF |
| ID | XP_ | lyrata | | DFKSYMINKAKSVNAALDISVPLRE |
| NO: | 020888857.1 | subsp. | | PLTVQEAVRYSLLAGGKRVRPMLCI |
| 305 | | lyrata | | ASCELVGGNEATAMPAACAVEMIHT |
| | | | | SSLIHDDLPCMDNADLRRGKPTNHK |
| | | | | VFGEDMAVLAGDALLALAFEHMTVV |
| | | | | SSGLVPPERMIRAVAELARAIGTTG |
| | | | | LVAGQMIDLASERLNPHDAGLERLE |
| | | | | FIHLHKTAALLEAAAVLGVIMGGGT |
| | | | | EEEIEKLRKYARCIGLLFQVVDDIL |
| | | | | DVTESTEELGKTAGKDLMAGKLTYP |
| | | | | RLIGLERSREVAEKLRKEAEEQVLG |
| | | | | FDSENSAPLVALASYIACRHN |
| SEQ | At.GGPPS_ | Arabidopsis | Yes | MSVVTKEDNLRQSEPSSEDEMSYII |
| ID | P34802_ | thaliana | | TKAELVNKALDSAVPLREPLKIHEA |
| NO: | L155M | | | MRYSLLAGGKRVRPVLCIAACELVG |
| 306 | | | | GEESTAMPAACAVEMIHTMSMIHDD |
| | | | | LPCMDNDDLRRGKPTNHKVFGEDVA |
| | | | | VLAGDALLSFAFEHLASATSSDVVS |
| | | | | PVRVVRAVGELAKAIGTEGLVAGQV |
| | | | | VDISSEGLDLNDVGLEHLEFIHLHK |
| | | | | TAALLEASAVLGAIVGGGSDDEIER |
| | | | | LRKFARCIGLLFQVVDDILDVTKSS |
| | | | | KELGKTAGKDLIADKLTYPKIMGLE |
| | | | | KSREFAEKLNREARDQLLGFDSDKV |
| | | | | APLLALANYIAYRQN |
| SEQ | Le.GGPPS_ | Loddero- | No | MDYKKVVNSLAQPTVVDPAVIEPYK |
| ID | XP_ | myces | | YIRKLASNNNNVRSRFINAFNETFF |
| NO: | 001524890.1 | elong- | | HITDQEVLQLINEVIEILHNASLLI |
| 307 | | isporus | | DDVEDSSNFRRGYPSAHMKYGVPLT |
| | | NRRL YB- | | INCGNLMYFEAIEKVYNLWRLKLGK |
| | | 4239 | | EDTTLATLALVWEIVMPEMKNFHKG |
| | | | | QGLDIYWRDNLPDLESLPSVVDYCQ |
| | | | | MAKDKTGGLERLAVRLLRLFSSGST |
| | | | | HLDSEYNGVFESIANCLGVIYQIRD |
| | | | | DYLNIVDERYSHMKGIRGEDLIEGK |
| | | | | LSFPILVSLTKDFNQKHTEISSLSP |
| | | | | LYELLYNHRSTEERLRRRDLHDQAI |
| | | | | EQLESPDIVSETNRLLQELKAQACR |
| | | | | LIMENARDANPLMIMVDTLCTI |
| SEQ | Ct.GGPPS_ | Chlorobium | No | MSSPITQAQVESKYRQYHAKINEAL |
| ID | WP_ | tepidum | | AACFPKEKPATLYDPARYILEGKGK |
| NO: | 010931948.1 | TLS | | RIRPFLTLLAAEAVSGKSDNALGVA |
| 308 | | | | LGIEVLHNFTLMHDDIMDQADLRHG |
| | | | | RPTVHKQWNVNAAILSGDMMIAYAY |
| | | | | ELALKAISSRHAEIIHIENDANITI |
| | | | | CEGQALDMELEQRKDVTIADYLDMI |
| | | | | SKKTGRLISAALEAGGVAGDGTPEQ |
| | | | | IAALVTFGEKIGRAFQIQDDYLDIM |
| | | | | AGDGKSGKVPGGDVINGKKTWLLLR |
| | | | | SLELAEGADRELLQSIFDNNGTSPD |
| | | | | NVPAVKAIFEKCGVLNETRAKINED |
| | | | | TEAALAALDALPFEEGRGYLRGFAN |
| | | | | ILMKRDF |
| SEQ | Pa.GGPPS_ | Picea | Yes | MELVNESENKDEEKPIAFEFKKYMF |
| ID | B1A9L0 | abies | | SKANAINEALDKCVSLRHPEKIHEA |
| NO: | | | | MRYSLLAGGKRVRPILCIASCELVG |
| 309 | | | | GSEESVMPSACAVEMIHTMSLIHDD |
| | | | | LPCMDNDDLRRGKPTNHKVFGEDVA |
| | | | | VLAGDALLAFAFEHIACSTKGVEAE |
| | | | | KVLRVIWELGRAIGSEGLVAGQVVD |
| | | | | LSSGGLSNVGLDLLEYIHVHKTAAL |
| | | | | LEGSVVIGAIIGGGSDEEIERLRRF |
| | | | | ARCIGLMFQVVDDILDVTKSSQELG |
| | | | | KTACKDLVADKLTYPKLLGLEKSRE |
| | | | | FAQELNKQAMDQISVEDVEKAKPLI |
| | | | | CLADYIAHRQN |
| SEQ | Tm.GGPPS_ | Thermotoga | No | MKKEKVEERIREILRPGWDLLTEEA |
| ID | WP_ | maritima | | MLYSATVGGKRIRPLLVLTLGEDLG |
| NO: | 004082783.1 | MSB8 | | VEEEKLLDVAVAVELFHTASLIHDD |
| 310 | | | | LPPIDNADFRRGKPSCHRTYGEDIA |
| | | | | LLAGDGLFFLAFSQISKIGNSKIFE |
| | | | | EFSETAYKLLLGEAMDVEFERRKME |
| | | | | VSQEMVERMYAFKTGALFAFCFSAP |
| | | | | FILKGKDHTKMKLLGEKFGVAFQIY |
| | | | | DDLKDILGSFEKVGKDLGKDTEKVT |
| | | | | LVKKVGIQKAREMADKYYEEVLKGI |
| | | | | ESEGLERTLFLLKELKQMVEER |
| SEQ | Mb.GGPPS_ | Myco- | No | MRTPATVVAGVDLGDAVFAAAVRAG |
| ID | O06428 | bacterium | | VARVEQLMDTELRQADEVMSDSLLH |
| NO: | | bovis | | LFNAGGKRFRPLFTVLSAQIGPQPD |
| 311 | | AF2122/97 | | AAAVTVAGAVIEMIHLATLYHDDVM |
| | | | | DEAQVRRGAPSANAQWGNNVAILAG |
| | | | | DYLLATASRLVARLGPEAVRIIADT |
| | | | | FAQLVTGQMRETRGTSENVDSIEQY |
| | | | | LKVVQEKTGSLIGAAGRLGGMFSGA |
| | | | | TDEQVERLSRLGGVVGTAFQIADDI |
| | | | | IDIDSESDESGKLPGTDVREGVHTL |
| | | | | PMLYALRESGPDCARLRALLNGPVD |
| | | | | DDAEVREALTLLRASPGMARAKDVL |
| | | | | AQYAAQARHELALLPDVPGRRALAA |
| | | | | LVDYTVSRHG |
| SEQ | Pa.GGPPS_ | Picea | Yes | MQLANLREEVKEVIKFDFKEYLLSK |
| ID | B1A9K9 | abies | | AMAVNEALDRAVQLRYPEKIHEAMR |
| NO: | | | | YSLLAGGKRVRPVLCIAACELVGGT |
| 312 | | | | EKLAMPTACAMEMIHTMSLIHDDLP |
| | | | | CMDNDDFRRGKPTNHKVFGEGTAVL |
| | | | | AGDALLSFAFEHIAVSTSKSVGSDR |
| | | | | ILRVVSELGRTIGSQGLVGGQVADI |
| | | | | TSEGNASVDLDTLEWIHIHKTAVLL |
| | | | | ECSVVCGAIISGASENEIERVKRYA |
| | | | | RSVGLLFQVVDDILDVTKSSKELGK |
| | | | | TAGKDLISDKATYPKLMGLEKAKQF |
| | | | | AVELLDRAKENLSCFDPMKAAPLLG |
| | | | | LADYIAFRQN |
| SEQ | Es.GGPPS_ | Eutrema- | Yes | MHFNIQSLTSVTRNKFDEMSYMVNK |
| ID | XP_ | salsugineum | | AKSVNKALDEAVPLCEPVLKIREAM |
| NO: | 006406415.1 | | | RYTLLSGGKRVRPMLCLAACELVGG |
| 313 | | | | QESTAMPAACAIEMLHASSLIQDDL |
| | | | | PCMDDDNLRRGKPTNHKVFGEGIAV |
| | | | | LTADALIALAVKKMATSSTSSPEKV |
| | | | | LRAILEMAKAVGTEGLVAGQAADLA |
| | | | | GEGMSLEENDDVGLKHLEFIHIHKT |
| | | | | AAMLEAAAVIGGIMGGGSDEEIERL |
| | | | | RSYARCIGLMFQVVDDVLDVTKTSE |
| | | | | ELGKTAGKDLIAGKLTYPKVMGVEK |
| | | | | SKEYAERLNREAREHLQGFDSDKVA |
| | | | | PLLFLADYIVNRQN |
| SEQ | Ml.GGPPS_ | Melampsora | No | MKPLPSTNGKVNGNGKHHDSSLSST |
| ID | QIG55792.1 | lini | | SSTSSSSSSDTQFNISDRYGDELHR |
| NO: | | | | LDTLDTWPKSNEQILLEPYTYLNNI |
| 314 | | | | PGKEIRSMMIDAFNHWLQIPRPALE |
| | | | | IIKKIVGQLHTASLLMDDVEDDSDL |
| | | | | RRGVPVTHKIYGIPQTINTANYVYF |
| | | | | LAYQELSKLKPCLSSNASTDLWSLV |
| | | | | NDELLQLHRGQGMDLYWRDSLTCPT |
| | | | | EEEYLQMVNNKTGGLFRIAIKLMIA |
| | | | | LSPLTETPDYLPLVNLVGIIFQIRD |
| | | | | DLLNLSSVYTKNKGFCEDLTEGKFS |
| | | | | FPIVHSIRADSSNHQLMNILRQKPT |
| | | | | DIGTKTFAVSYMKDQTKSLQYTREV |
| | | | | LTCLEEQAIEEVTRLGGNPALESIF |
| | | | | ELMHVLPSPPATDQH |
| SEQ | Pc.GGPPS_ | Pyrobaculum | No | MDVVSRLHQKYGAEVEKALVRYLSI |
| ID | WP_ | calidi- | | GLAEDFREAVLYQVKTGGKRLRPLL |
| NO: | 011848845.1 | fontis | | TLAAAEAVSGQWRPALPAAAIVELI |
| 315 | | JCM | | HNYSLIYDDIIDRGDVRRGLPTVRK |
| | | 11548 | | AFGDNAAILVGIWYREAIEEAVLDT |
| | | | | PKPTLFAKEVAEVIKAIDEGERLDI |
| | | | | LFEAAGRSDPYFVQARWREVTLDDY |
| | | | | IKMVSLKTGALIAAAAKWGVLSVSD |
| | | | | DRGLAEAAWNFGMAAGVAFQIIDDV |
| | | | | LDIYGDPKKFGKEIGKDIKEHKRGN |
| | | | | AVVAVALSHLGEGERRRLLEILARE |
| | | | | VVEEADVREAVALLDSVGAREEALR |
| | | | | LAARYREEAERHLAKIPNNGTLKEL |
| | | | | LDFIVAREY |
| SEQ | Cg.GGPPS_ | Coryne- | No | MRDNWPCDTRMDNGMTITTEHSTHP |
| ID | WP_ | bacterium | | DLDENDEIYRELNRICASLSQQCST |
| NO: | 011013776.1 | glutamicum | | YQPEFRTCLDAAFQALRGGKLIRPR |
| 316 | | ATCC | | MLLGLYNTLVDDDIEVKLNTVLQVA |
| | | 13032 | | VALELLHFSLLVHDDVIDGDLYRRG |
| | | | | KLNFIGQILMHRTPESFAQIQRDPE |
| | | | | HLDWAQSNGLLMGNLFLAATHQIFA |
| | | | | RLDLPHHQRVRLLDLLNHTINDTIV |
| | | | | GEFLDVGLSSKAISPNMDIALEMSR |
| | | | | LKTATYTFELPMRAAAILAELPQEI |
| | | | | ETKIGEIGTNLGIAYQLQDDYLSTE |
| | | | | GDAAEHGKDAFSDLREGKETTIIAF |
| | | | | ARDTAKWTDIQDNFGSADLSTSQAE |
| | | | | RIQHLLIQCGAKNHSLNAISDHLNI |
| | | | | CRSMIKTLSPQVDPKAQNLLLKQVE |
| | | | | QLASRKS |
| SEQ | Lc.GGPPS_ | Litsea | Yes | MVIEGEKENDSSYSETTPPFDLKTY |
| ID | ART33318.1 | cubeba | | MRQKGNSVNRALEEAVPLRSPEALY |
| NO: | | | | EPMRYSLFAGGKRVRPLLCISACEL |
| 317 | | | | VGGNEATAMPSACAAEMIHTMSLMH |
| | | | | DDLPYMDDDDLRRGKPTAHKAFGET |
| | | | | AAVLSGDVMLLLAFEHVAAATPGAP |
| | | | | PDRVARAVGELARAAGRDGLVAGQI |
| | | | | VDVCSEGSGEVGLERLEYIHLHKTA |
| | | | | ALLEGAVVAGGMLGGGTEEEIGRLR |
| | | | | RFARCIGLLFQVVDDILDVTKSSTE |
| | | | | LGKTAGKDVAAEKVTYPKMLGLEGS |
| | | | | RELAEELRKEAKEQLMGFDPEKAMP |
| | | | | LVALTDFIACRNN |
| SEQ | Ll.GGPPS_ | Lactococcus | No | MRLISKEHKIFGIIRVMDTKILKLE |
| ID | WP_ | lactis | | HELTEFYESAEFPKGLAEPAKYSLM |
| NO: | 011835496.1 | subsp. | | AGGKRIRPLLFLNLLEAFDLEVTTA |
| 318 | | cremoris | | HYQVAAALEMIHTGSLIHDDLPAMD |
| | | MG1363 | | NDDYRRGKLTNHKKEDEATAILAGD |
| | | | | TLFFDPFYVLSSSDLSAQTIVSLTR |
| | | | | ELAYASGSYGMVAGQILDMAGEGKD |
| | | | | LTLTQIEQIHRLKTGRLLTFPFVAA |
| | | | | GIVAQKTNQEIEKLRVVGQILGLAF |
| | | | | QIRDDILDVTATFAELGKTPGKDVL |
| | | | | EEKSTYVAHLGLEGAKISLTDKLSE |
| | | | | VKKLLTELNVTDSSEIFKIIEQLEV |
| | | | | E |
| SEQ | Lp.GGPPS_ | Legionella | No | MVLIPTLNSTQMNKQVIDKYTQRHE |
| ID | WP_ | pneumophila | | LYLEQLLNEIIIPAPQIRSALHYAL |
| NO: | 010948036.1 | subsp. | | FSGGKRIRPILVYLAGDLIDVDQGV |
| 319 | | pneumophila | | LDIIAAALELTHCYSLIHDDLPAMD |
| | | str. | | NDDLRRGKPSCHKAFDEATAILVGD |
| | | Phila- | | GMQALAIEVLLMRLSPLLPAAQVVA |
| | | delphia 1 | | ITQVLVNASGISGMVSGQSLDLSEL |
| | | | | AKSSVTEEQLREIHLLKTGKLILAC |
| | | | | FEMVLAAQHEVSEQIKSALRTYGKH |
| | | | | IGLVFQMQDDYLDLYAPTQILGKGR |
| | | | | SSDQANQKTTFATLENKQQLEEEIA |
| | | | | VHYQIAMDSLRLFGSKAAALIELTK |
| | | | | QLQNRSNLS |
| SEQ | Ns.GGPPS_ | Nicotiana | Yes | MEAEVIKSENTFREAGKTKTVFDFK |
| ID | XP_ | sylvestris | | SYMYQKINSVNRALDAAVPLREPLK |
| NO: | 009784772.1 | | | FHEAMRYSLLSEGKRVCPVLCIAAC |
| 320 | | | | ELVGGQESTAMPAACGMEMIHAMCM |
| | | | | MHDDLPCMDNDNLRRGKLANHKVFG |
| | | | | ENVTVLAGYSLVALAFEHMATATKG |
| | | | | VHPKTMVRAIGELARLIGPEGAAAG |
| | | | | QVLDLLSGGESDTGLEELEYIHRHK |
| | | | | TADFAEAAVIVGAMVGGATDEEINR |
| | | | | LRKFSQCIGLLFQVVDDILDVTKSS |
| | | | | EQLGKTAGKDLLANKLTYPKMIGIE |
| | | | | KSKEYAQNLSKEAKEQLVGFDPEKA |
| | | | | APLLAMADFVLHRQK |
| SEQ | Pf.GGPPS_ | Perilla | Yes | MIRFISNKIHQNDQFDFKAYMLEKI |
| ID | KAH6803897. | frutescens | | TAVNQALDAALPLQEPTKLHEAMRY |
| NO: | 1 | var. | | SLLSGGKRICPIVCLAACRLVGGNE |
| 321 | | frutescens | | AAAMPSACALEMIHAMSLMHDDLPC |
| | | | | MDNDDLRRGRPSSHKVFGEGAAVLA |
| | | | | GYALIATAFDHIATATKGVAEGRIV |
| | | | | RVVGELARLIGPEGVVAGQVADLQC |
| | | | | GGRGRKDIGLEQLEYIHLQKTAASV |
| | | | | EASAVAGAIIGGASEEEIEKLRKYS |
| | | | | RRAGLLFQVVDDILDVTKSSEELGK |
| | | | | TAGKDLAAEKATYPKLIGVEKSREL |
| | | | | AEQLKREAHQQLLGFDGVRAAPLIA |
| | | | | LLHFIAYRDK |
| SEQ | Ph.GGPPS_ | Paracoccus | No | MRRDVNPIHATLLQTRLEEIAQGFG |
| ID | AAY28422.1 | haeund- | | AVSQPLGAAMSHGALSSGRRFRGML |
| No: | | aensis | | MLLAAEASGGVCDTIVDAACAVEMV |
| 322 | | | | HAASLIFDDLPCMDDAGLRRGRPAT |
| | | | | HVAHGESRAVLGGIALITEAMALLA |
| | | | | GARGASGTVRAQLVRILSRSLGPQG |
| | | | | LCAGQDLDLHAAKNGAGVEQEQDLK |
| | | | | TGVLFIAGLEMLAVIKEFDAEEQTQ |
| | | | | MIDFGRQLGRVFQSYDDLLDVVGDQ |
| | | | | AALGKDTGRDAAAPGPRRGLLAVSD |
| | | | | LQNVSRHYEASRAQLDAMLRSKRLQ |
| | | | | APEIAALLERVLPYAARA |
| SEQ | Sp.GGPPS_ | Strepto- | No | MKKQEKLALVESVLEDFYGDQQFAS |
| ID | ZP_ | coccus | | SLRESVLYSIHAGGKRIRPFLLLEV |
| NO: | 01823217.1 | pneumoniae | | LEALQVAIKPAHAQVATALEMIHTG |
| 323 | | | | SLIHDDLPAMDDDDYRRGRLTNHKK |
| | | | | FGEAMAILAGDALFLDPYALIAQAD |
| | | | | LPSQIKVDLIASLSLASGSLGMVAG |
| | | | | QVLDMEGEHQHLSLEELQTIHANKT |
| | | | | GKLLAYPFQAAAIIAELSPEMQVKL |
| | | | | KTVGELIGLAFQVRDDVLDVTASFE |
| | | | | EIGKTPQKDLQAEKSTYPALLGLEE |
| | | | | SIVFCNQTLDQANEKLEEIAQQVPF |
| | | | | ETESIVSVVESLRING |
| SEQ | Ss.GGPPS_ | Shigella | No | MDFPQQLEACVKQANQALSRFIAPL |
| ID | NP_706309.1 | sonnei | | PFQNTPVVETMQYGALLGGKRLRPF |
| NO: | | | | LVYATGHMFGVSTNTLDAPAAAVEC |
| 324 | | | | IHAYSLIHDDLPAMDDDDLRRGLPT |
| | | | | CHVKFGEANAILAGDALQTLAFSIL |
| | | | | SDANMPEVSDRDRISMISELASASG |
| | | | | IAGMCGGQALDLDAEGKHVPLDALE |
| | | | | RIHRHKTGALIRAAVRLGALSAGDK |
| | | | | GRRALPVLDKYAESIGLAFQVQDDI |
| | | | | LDVVGDTATLGKRQGADQQLGKSTY |
| | | | | PALLGLEQARKKARDLIDDARQALK |
| | | | | QLAEQSLDTSALEALADYIIQRNK |
| SEQ | Um.GGPPS_ | uncultured | No | MENGLLDCEQYLEEAMAEHATAQCP |
| ID | AAR37858.1 | marine | | PLLAQALNYAVFPGGARVRPKICKA |
| NO: | | proteo- | | VALANNSSDVGLANAAASAIELLHC |
| 325 | | bacterium | | ASLVHDDLPCFDDATQRRGKPSVHA |
| | | | | KFGERIAVLTGDALIVAAFQTLATH |
| | | | | AIHAVRTERVPLVTAIVARGVGAPH |
| | | | | GICAGQAWECERSVDLSRYHRAKTG |
| | | | | ALFVAATCAGAAAAGVDPGPWVNLG |
| | | | | ASIGEAYQVADDIKDAISDPETLGK |
| | | | | PTGIDVKLDRPSAVRELGLDGAVTR |
| | | | | LKQCLEAGLDSMPACAGQDLLQKIV |
| | | | | RAQASRFVPEKIAQVAA |
| SEQ | Vr.GGPPS_ | Vigna | Yes | MLSPPDTVRREEIGFDFKAYMQHKI |
| ID | XP_ | radiata | | NTVNRALDAAIELKEPKNIHEAMRY |
| NO: | 022634843.1 | var. | | TLLAGGKKIRPVLCIAACELVGGTD |
| 326 | | radiata | | YAAVPAACAVEMVHAMSLIHDDLPS |
| | | | | IDNDDLRRGKPACHKVFGENVAILA |
| | | | | GDALQAYAFEHLAASTKGVSPTRVV |
| | | | | RAIVELAKSVGAEGVAGGQVMDVSS |
| | | | | EGLSSEVGLERVELIHLKKSAALLE |
| | | | | GSVVLGAIVGGGSDEEVERLRMFGR |
| | | | | CVGLMGQIVDDIVDVTKSTEELGKT |
| | | | | AGKDLVVDKLTYPKVFGMHKSKEIA |
| | | | | QKLVEDSTEILVGEDNPKVAPLVAL |
| | | | | TNYIAGKIN |
| SEQ | Nb.GGPPS_ | brasilie | No | MKPGYIDSRTEEFDPAPALRHYLAG |
| ID | B1Q2P2 | Nocard- | | TAAGDGDRLDAVTRYAVQPPGKLVR |
| NO: | | niasis | | PVLLVESAAAVGADPADLVPIAVAL |
| 327 | | | | EYLHVATLVHDDIIDGDLLRRGRPT |
| | | | | VHARYGRDEAIVAGDALLLDTERTL |
| | | | | MAGLKTGALFIAACAAGAVLGGGSG |
| | | | | DLCRGQAMEAGLAGDLYCGMARYRT |
| | | | | EEVRALRSFADHLGCAFQMRDDLLP |
| | | | | TEAAELGFPPAAVLEAVRIVAVAGL |
| | | | | YLADTSRAGKSSVTDIVNORPTFPV |
| | | | | LVGIELADTTQCWRIEQSLSGRLPA |
| | | | | DEAHRLLRQVLTETGALAEASARVA |
| | | | | AEVAAARADLAPFGVRGERLIALAD |
| | | | | SMIGRTW |

1. A recombinant host cell capable of producing retinol comprising a heterologous nucleic acid that encodes a first polypeptide having a sequence having at least 90% identity to a sequence selected from SEQ ID NO: 10, SEQ ID NO: 153, SEQ ID NO: 154, SEQ ID NO: 155, SEQ ID NO: 156, and any one of SEQ ID NOs: 217-240; a heterologous nucleic acid that encodes a second polypeptide having a sequence having at least 90% identity to a sequence selected from SEQ ID NO: 11, SEQ ID NO: 150, SEQ ID NO: 151, SEQ ID NO: 152, and any one of SEQ ID NOs: 158-216; a heterologous nucleic acid that encodes a third polypeptide having a sequence having at least 90% identity to a sequence selected from SEQ ID NOs: 12 and 55-149; and a heterologous nucleic acid that encodes a fourth polypeptide having a sequence having at least 90% identity to a sequence selected from SEQ ID NOs: 14-54.

2. The recombinant host cell of claim 1, wherein the first polypeptide has a sequence selected from SEQ ID NO: 10, SEQ ID NO: 153, SEQ ID NO: 154, SEQ ID NO: 155, SEQ ID NO: 156, and any one of SEQ ID NOs: 217-240, and wherein the second polypeptide has a sequence selected from SEQ ID NO: 11, SEQ ID NO: 150, SEQ ID NO: 151, SEQ ID NO: 152, and any one of SEQ ID NOs: 158-216 and wherein the third polypeptide has a sequence selected from SEQ ID NOs: 12 and 55-149, and wherein the fourth polypeptide has a sequence selected from SEQ ID NOs: 14-54.

3. A recombinant host cell capable of producing lycopene comprising a heterologous nucleic acid that encodes a phytoene synthase, and comprising a heterologous nucleic acid that encodes a polypeptide having a sequence having at least 90% identity to a sequence selected from SEQ ID NO: 11, SEQ ID NO: 150, SEQ ID NO: 151, SEQ ID NO: 152, and any one of SEQ ID NOs: 158-216.

4. The recombinant host cell of claim 3, wherein the polypeptide has a sequence selected from SEQ ID NO: 11, SEQ ID NO: 150, SEQ ID NO: 151, SEQ ID NO: 152, any one of SEQ ID NOs: 158-216.

5. A recombinant host cell capable of producing beta-carotene comprising a heterologous nucleic acid that encodes a first polypeptide having a sequence having at least 90% identity to a sequence selected from SEQ ID NO: 10, SEQ ID NO: 153, SEQ ID NO: 154, SEQ ID NO: 155, SEQ ID NO: 156, and any one of SEQ ID NOs: 217-240, and comprising a heterologous nucleic acid that encodes a second polypeptide having a sequence having at least 90% identity to a sequence selected from SEQ ID NO: 11, SEQ ID NO: 150, SEQ ID NO: 151, SEQ ID NO: 152, and any one of SEQ ID NOs: 158-216.

6. The recombinant host cell of claim 5, wherein the first polypeptide has a sequence selected from SEQ ID NO: 10, SEQ ID NO: 153, SEQ ID NO: 154, SEQ ID NO: 155, SEQ ID NO: 156, and any one of SEQ ID NOs: 217-240, and wherein the second polypeptide has a sequence selected from SEQ ID NO: 11, SEQ ID NO: 150, SEQ ID NO: 151, SEQ ID NO: 152, and any one of SEQ ID NOs: 158-216.

7. A recombinant host cell capable of producing retinal comprising a heterologous nucleic acid that encodes a first polypeptide having a sequence having at least 90% identity to a sequence selected from SEQ ID NO: 10, SEQ ID NO: 153, SEQ ID NO: 154, SEQ ID NO: 155, SEQ ID NO: 156, and any one of SEQ ID NOs: 217-240; a heterologous nucleic acid that encodes a second polypeptide having a sequence having at least 90% identity to a sequence selected from SEQ ID NO: 11, SEQ ID NO: 150, SEQ ID NO: 151, SEQ ID NO: 152, and any one of SEQ ID NOs: 158-216; and a heterologous nucleic acid that encodes a third polypeptide having a sequence having at least 90% identity to a sequence selected from SEQ ID NOs: 12 and 55-149.

8. The recombinant host cell of claim 7, wherein the first polypeptide has a sequence selected from SEQ ID NO: 10, SEQ ID NO: 153, SEQ ID NO: 154, SEQ ID NO: 155, SEQ ID NO: 156, and any one of SEQ ID NOs: 217-240, the second polypeptide has a sequence selected from SEQ ID NO: 11, SEQ ID NO: 150, SEQ ID NO: 151, SEQ ID NO: 152, and any one of SEQ ID NOs: 158-216, and the third polypeptide has a sequence selected from SEQ ID NOs: 12 and 55-149.

9. A recombinant host cell capable of producing phytoene comprising a heterologous nucleic acid that encodes a first polypeptide having at least 90% sequence identity to a sequence selected from SEQ ID NO: 10, SEQ ID NO: 153, SEQ ID NO: 154, SEQ ID NO: 155, SEQ ID NO: 156, and any one of SEQ ID NOs: 217-240.

10. The recombinant host cell of any of the preceding claims further comprising one or more heterologous nucleic acids that encode one or more polypeptides having a sequence having at least 90% identity to a sequence selected from SEQ ID NO: 3, SEQ ID NO: 1, SEQ ID NO: 7, SEQ ID NO: 157, SEQ ID NO: 2, SEQ ID NO: 8, SEQ ID NO: 5, SEQ ID NO: 6, and SEQ ID NO: 4.

11. The recombinant host cell of claim 10, wherein the one or more polypeptides have a sequence selected from SEQ ID NO: 3, SEQ ID NO: 1, SEQ ID NO: 7, SEQ ID NO: 157, SEQ ID NO: 2, SEQ ID NO: 8, SEQ ID NO: 5, SEQ ID NO: 6, and SEQ ID NO: 4.

12. The recombinant host cell of any one of the preceding claims, further comprising a heterologous nucleic acid encoding a geranylgeranyl diphosphate synthase having a sequence having at least 90% identity to a sequence selected from SEQ ID NOs: 9 and 289-310.

13. The recombinant host cell of claim 12, wherein the geranylgeranyl diphosphate synthase has a sequence selected from SEQ ID NOs: 9 and 289-310.

14. The recombinant host cell of any one of the preceding claims, wherein the recombinant host cell comprises a deletion of at least a portion of a native alcohol dehydrogenase gene.

15. The recombinant host cell of claim 14, wherein the native alcohol dehydrogenase gene has a sequence having at least 90% identity to SEQ ID NO: 13.

16. The recombinant host cell of any one of the preceding claims, wherein the recombinant host cell further comprises an ERG9 gene that is downregulated relative to the parent strain.

17. The recombinant host cell of any one of the preceding claims, wherein the recombinant host cell further comprises a heterologous nucleic acid encoding a lycopene cyclase, wherein the lycopene cyclase has a sequence having at least 90% identity to a sequence selected from any one of SEQ ID NOs: 243-273.

18. The recombinant host cell of any one of the preceding claims, wherein the recombinant host cell further comprises a heterologous nucleic acid encoding a lycopene cyclase, wherein the lycopene cyclase has a sequence selected from any one of SEQ ID NOs: 243-273.

19. The recombinant host cell of any one of the preceding claims, wherein the host cell comprises a plant cell, a yeast cell, or a bacterial cell.

20. The recombinant host cell of claim 19, wherein the host cell is a yeast cell.

21. The recombinant host cell of claim 20, wherein the host cell is a Saccharomyces cerevisiae cell.

22. A method of producing retinol comprising: culturing a population of recombinant host cells of any one of claims 1, 2, and 10-21 in a culture medium comprising a carbon source under conditions suitable for making retinol; and recovering the retinol from the culture medium.

23. The method of claim 22, further comprising providing an overlay, and wherein the retinol is recovered from the culture medium or the overlay.

24. A method of producing lycopene comprising: culturing a population of recombinant host cells of any one of claims 3, 4, and 10-21 in a culture medium comprising a carbon source under conditions suitable for making lycopene; and recovering the lycopene from the culture medium.

25. The method of claim 24, further comprising providing an overlay, and wherein the lycopene is recovered from the culture medium or the overlay.

26. A method of producing beta-carotene comprising: culturing a population of recombinant host cells of any one of claims 5, 6, and 10-21 in a culture medium comprising a carbon source under conditions suitable for making beta-carotene; and recovering the beta-carotene from the culture medium.

27. The method of claim 26, further comprising providing an overlay, and wherein the beta-carotene is recovered from the culture medium or the overlay.

28. A method of producing retinal comprising: culturing a population of recombinant host cells of any one of claims 7, 8, and 10-21 in a culture medium comprising a carbon source under conditions suitable for making retinal; and recovering the retinal from the culture medium.

29. The method of claim 28, further comprising providing an overlay, and wherein the retinal is recovered from the culture medium or the overlay.

30. A method for producing phytoene comprising: culturing a population of recombinant host cells of any one of claims 9-21 in a culture medium comprising a carbon source under conditions suitable for making phytoene; and recovering the phytoene from the culture medium.

31. The method of claim 30, further comprising providing an overlay, and wherein the phytoene is recovered from the culture medium or overlay.


SEQUENCE LISTING (327 sequences)

SEQ ID NO: 1 - protein, 398 aa, Saccharomyces cerevisiae
  REGION 1..398: note = misc_feature - Acetyl-CoA acetyltransferase P41338
MSQNVYIVSTARTPIGSFQGSLSSKTAVELGAVALKGALAKVPELDASKDFDEIIFGNVLSANLGQAPARQVALAAGLSNHIVASTVNKVCASAMKAIILGAQSIKCGNADVVVAGGCESMTNAPYYMPAARAGAKFGQTVLVDGVERDGLNDAYDGLAMGVHAEKCARDWDITREQQDNFAIESYQKSQKSQKEGKFDNEIVPVTIKGFRGKPDTQVTKDEEPARLHVEKLRSARTVFQKENGTVTAANASPINDGAAAVILVSEKVLKEKNLKPLAIIKGWGEAAHQPADFTWAPSLAVPKALKHAGIEDINSVDYFEFNEAFSVVGLVNTKILKLDPSKVNVYGGAVALGHPLGCSGARVVVTLLSILQQEGGKIGVAAICNGGGGASSIVIEKI

SEQ ID NO: 2 - protein, 491 aa, Saccharomyces cerevisiae
  REGION 1..491: note = misc_feature - Hydroxymethylglutaryl-CoA synthase P54839
MKLSTKLCWCGIKGRLRPQKQQQLHNTNLQMTELKKQKTAEQKTRPQNVGIKGIQIYIPTQCVNQSELEKFDGVSQGKYTIGLGQTNMSFVNDREDIYSMSLTVLSKLIKSYNIDTNKIGRLEVGTETLIDKSKSVKSVLMQLFGENTDVEGIDTLNACYGGTNALFNSLNWIESNAWDGRDAIVVCGDIAIYDKGAARPTGGAGTVAMWIGPDAPIVFDSVRASYMEHAYDFYKPDFTSEYPYVDGHFSLTCYVKALDQVYKSYSKKAISKGLVSDPAGSDALNVLKYFDYNVFHVPTCKLVTKSYGRLLYNDFRANPQLFPEVDAELATRDYDESLTDKNIEKTFVNVAKPFHKERVAQSLIVPTNTGNMYTASVYAAFASLLNYVGSDDLQGKRVGLFSYGSGLAASLYSCKIVGDVQHIIKELDITNKLAKRITETPKDYEAAIELRENAHLKKNFKPQGSIEHLQSGVYYLTNIDDKFRRSYDVKK

SEQ ID NO: 3 - protein, 1045 aa, Saccharomyces cerevisiae
  REGION 1..1045: note = misc_feature - 3-hydroxy-3-methylglutaryl-coenzyme A reductase 2 P12684
MSLPLKTIVHLVKPFACTARFSARYPIHVIVVAVLLSAAAYLSVTQSYLNEWKLDSNQYSTYLSIKPDELFEKCTHYYRSPVSDTWKLLSSKEAADIYTPFHYYLSTISFQSKDNSTTLPSLDDVIYSVDHTRYLLSEEPKIPTELVSENGTKWRLRNNSNFILDLHNIYRNMVKQFSNKTSEFDQFDLFIILAAYLTLFYTLCCLFNDMRKIGSKFWLSFSALSNSACALYLSLYTTHSLLKKPASLLSLVIGLPFIVVIIGFKHKVRLAAFSLQKFHRISIDKKITVSNIIYEAMFQEGAYLIRDYLFYISSFIGCAIYARHLPGLVNFCILSTFMLVFDLLLSATFYSAILSMKLEINIIHRSTVIRQTLEEDGVVPTTADIIYKDETASEPHFLRSNVAIILGKASVIGLLLLINLYVFTDKLNATILNTVYFDSTIYSLPNFINYKDIGNLSNQVIISVLPKQYYTPLKKYHQIEDSVLLIIDSVSNAIRDQFISKLLFFAFAVSISINVYLLNAAKIHTGYMNFQPQSNKIDDLVVQQKSATIEFSETRSMPASSGLETPVTAKDIIISEEIQNNECVYALSSQDEPIRPLSNLVELMEKEQLKNMNNTEVSNLVVNGKLPLYSLEKKLEDTTRAVLVRRKALSTLAESPILVSEKLPFRNYDYDRVFGACCENVIGYMPIPVGVIGPLIIDGTSYHIPMATTEGCLVASAMRGCKAINAGGGATTVLTKDGMTRGPVVRFPTLIRSGACKIWLDSEEGQNSIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVKSVLKSDVSALVELNISKNLVGSAMAGSVGGFNAHAANLVTALFLALGQDPAQNVESSNCITLMKEVDGDLRISVSMPSIEVGTIGGGTVLEPQGAMLDLLGVRGPHPTEPGANARQLARIIACAVLAGELSLCSALAAGHLVQSHMTHNRKTNKANELPQPSNKGPPCKTSALL

SEQ ID NO: 4 - protein, 1054 aa, Saccharomyces cerevisiae
  REGION 1..1054: note = misc_feature - 3-hydroxy-3-methylglutaryl-coenzyme A reductase 1 P12683
MPPLFKGLKQMAKPIAYVSRFSAKRPIHIILFSLIISAFAYLSVIQYYFNGWQLDSNSVFETAPNKDSNTLFQECSHYYRDSSLDGWVSITAHEASELPAPHHYYLLNLNFNSPNETDSIPELANTVFEKDNTKYILQEDLSVSKEISSTDGTKWRLRSDRKSLFDVKTLAYSLYDVFSENVTQADPFDVLIMVTAYLMMFYTIFGLFNDMRKTGSNFWLSASTVVNSASSLFLALYVTQCILGKEVSALTLFEGLPFIVVVVGFKHKIKIAQYALEKFERVGLSKRITTDEIVFESVSEEGGRLIQDHLLCIFAFIGCSMYAHQLKTLTNFCILSAFILIFELILTPTFYSAILALRLEMNVIHRSTIIKQTLEEDGVVPSTARIISKAEKKSVSSFLNLSVVVIIMKLSVILLFVFINFYNFGANWVNDAFNSLYFDKERVSLPDFITSNASENFKEQAIVSVTPLLYYKPIKSYQRIEDMVLLLLRNVSVAIRDRFVSKLVLSALVCSAVINVYLLNAARIHTSYTADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 5 - protein, 352 aa, unidentified
  REGION 1..352: note = farnesyl pyrophosphate synthase
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAINDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIDQLYHEYEESVAKDLKAKISQVDESRGFKADVLTAFLNKVYKRSK

SEQ ID NO: 6 - protein, 451 aa, Saccharomyces cerevisiae
  REGION 1..451: note = misc_feature - Phosphomevalonate kinase P24521
MSELRAFSAPGKALLAGGYLVLDTKYEAFVVGLSARMHAVAHPYGSLQGSDKFEVRVKSKQFKDGEWLYHISPKSGFIPVSIGGSKNPFIEKVIANVFSYFKPNMDDYCNRNLFVIDIFSDDAYHSQEDSVTEHRGNRRLSFHSHRIEEVPKTGLGSSAGLVTVLTTALASFFVSDLENNVDKYREVIHNLAQVAHCQAQGKIGSGFDVAAAAYGSIRYRRFPPALISNLPDIGSATYGSKLAHLVDEEDWNITIKSNHLPSGLTLWMGDIKNGSETVKLVQKVKNWYDSHMPESLKIYTELDHANSRFMDGLSKLDRLHETHDDYSDQIFESLERNDCTCQKYPEITEVRDAVATIRRSFRKITKESGADIEPPVQTSLLDDCQTLKGVLTCLIPGAGGYDAIAVITKQDVDLRAQTANDKRFSKVQWLDVTQADWGVRKEKDPETYLDK

SEQ ID NO: 7 - protein, 396 aa, Saccharomyces cerevisiae
  REGION 1..396: note = misc_feature - Diphosphomevalonate decarboxylase P32377
MTVYTASVTAPVNIATLKYWGKRDTKLNLPTNSSISVTLSQDDLRTLTSAATAPEFERDTLWLNGEPHSIDNERTQNCLRDLRQLRKEMESKDASLPTLSQWKLHIVSENNFPTAAGLASSAAGFAALVSAIAKLYQLPQSTSEISRIARKGSGSACRSLFGGYVAWEMGKAEDGHDSMAVQIADSSDWPQMKACVLVVSDIKKDVSSTQGMQLTVATSELFKERIEHVVPKRFEVMRKAIVEKDFATFAKETMMDSNSFHATCLDSFPPIFYMNDTSKRIISWCHTINQFYGETIVAYTFDAGPNAVLYYLAENESKLFAFIYKLFGSVPGWDKKFTTEQLEAFNHQFESSNFTARELDLELQKDVARVILTQVGSGPQETNESLIDAKTGLPKE

SEQ ID NO: 8 - protein, 288 aa, Saccharomyces cerevisiae
  REGION 1..288: note = misc_feature - Isopentenyl-diphosphate Delta-isomerase P15496
MTADNNSMPHGAVSSYAKLVQNQTPEDILEEFPEIIPLQQRPNTRSSETSNDESGETCFSGHDEEQIKLMNENCIVLDWDDNAIGAGTKKVCHLMENIEKGLLHRAFSVFIFNEQGELLLQQRATEKITFPDLWTNTCCSHPLCIDDELGLKGKLDDKIKGAITAAVRKLDHELGIPEDETKTRGKFHFLNRIHYMAPSNEPWGEHEIDYILFYKINAKENLTVNPNVNEVRDFKWVSPNDLKTMFADPSYKFTPWFKIICENYLFNWWEQLDDLSEVENDRQIHRML

SEQ ID NO: 9 - protein, 320 aa, unidentified
  REGION 1..320: note = Bt.GGPPS; GGPP Synthase
MLTSSKSIESFPKNVQPYGKHYQNGLEPVGKSQEDILLEPFHYLCSNPGKDVRTKMIEAFNAWLKVPKDDLIVITRVIEMLHSASLLIDDVEDDSVLRRGVPAAHHIYGTPQTINCANYVYFLALKEIAKLNKPNMITIYTDELINLHRGQGMELFWRDTLTCPTEKEFLDMVNDKTGGLLRLAVKLMQEASQSGTDYTGLVSKIGIHFQVRDDYMNLQSKNYADNKGFCEDLTEGKFSFPIIHSIRSDPSNRQLLNILKQRSSSIELKQFALQLLENTNTFQYCRDFLRVLEKEAREEIKLLGGNIMLEKIMDVLSVNE

SEQ ID NO: 10 - protein, 673 aa, Phaffia rhodozyma
  REGION 1..673: note = misc_feature - Xd.CrtYB_Q7Z859, Phaffia rhodozyma (Xanthophyllomyces dendrorhous)
MTALAYYQIHLIYTLPILGLLGLLTSPILTKFDIYKISILVFIAFSATTPWDSWIIRNGAWTYPSAESGQGVFGTFLDVPYEEYAFFVIQTVITGLVYVLATRHLLPSLALPKTRSSALSLALKALIPLPIIYLFTAHPSPSPDPLVTDHYFYMRALSLLITPPTMLLAALSGEYAFDWKSGRAKSTIAAIMIPTVYLIWVDYVAVGQDSWSINDEKIVGWRLGGVLPIEEAMFFLLTNLMIVLGLSACDHTQALYLLHGRTIYGNKKMPSSFPLITPPVLSLFFSSRPYSSQPKRDLELAVKLLEEKSRSFFVASAGFPSEVRERLVGLYAFCRVTDDLIDSPEVSSNPHATIDMVSDFLTLLFGPPLHPSQPDKILSSPLLPPSHPSRPTGMYPLPPPPSLSPAELVQFLTERVPVQYHFAFRLLAKLQGLIPRYPLDELLRGYTTDLIFPLSTEAVQARKTPIETTADLLDYGLCVAGSVAELLVYVSWASAPSQVPATIEEREAVLVASREMGTALQLVNIARDIKGDATEGRFYLPLSFFGLRDESKLAIPTDWTEPRPQDFDKLLSLSPSSTLPSSNASESFRFEWKTYSLPLVAYAEDLAKHSYKGIDRLPTEVQAGMRAACASYLLIGREIKVVWKGDVGERRTVAGWRRVRKVLSVVMSGWEGQ

SEQ ID NO: 11 - protein, 582 aa, Phaffia rhodozyma
  REGION 1..582: note = misc_feature - Xd.CrtI_O13506, Phaffia rhodozyma (Xanthophyllomyces dendrorhous)
MGKEQDQDKPTAIIVGCGIGGIATAARLAKEGFQVTVFEKNDYSGGRCSLIERDGYRFDQGPSLLLLPDLFKQTFEDLGEKMEDWVDLIKCEPNYVCHFHDEETFTFSTDMALLKREVERFEGKDGFDRFLSFIQEAHRHYELAVVHVLQKNFPGFAAFLRLQFIGQILALHPFESIWTRVCRYFKTDRLRRVFSFAVMYMGQSPYSAPGTYSLLQYTELTEGIWYPRGGFWQVPNTLLQIVKRNNPSAKFNFNAPVSQVLLSPAKDRATGVRLESGEEHHADVVIVNADLVYASEHLIPDDARNKIGQLGEVKRSWWADLVGGKKLKGSCSSLSFYWSMDRIVDGLGGHNIFLAEDFKGSFDTIFEELGLPADPSFYVNVPSRIDPSAAPEGKDAIVILVPCGHIDASNPQDYNKLVARARKFVIQTLSAKLGLPDFEKMIVAEKVHDAPSWEKEFNLKDGSILGLAHNFMQVLGFRPSTRHPKYDKLFFVGASTHPGTGVPIVLAGAKLTANQVLESFDRSPAPDPNMSLSVPYGKPLKSNGTGIDSQVQLKFMDLERWVYLLVLLIGAVIARSVGVLAF

SEQ ID NO: 12 - protein, 547 aa, unidentified
  REGION 1..547: note = Hs.BCDO; Beta-carotene 15-15 -dioxygenase
MDIIFGRNRKEQLEPVRAKVTGKIPAWLQGTLLRNGPGMHTVGESRYNHWFDGLALLHSFTIRDGEVYYRSKYLRSDTYNTNIEANRIVVSEFGTMAYPDPCKNIFSKAFSYLSHTIPDFTDNCLINIMKCGEDFYATSETNYIRKINPQTLETLEKVDYRKYVAVNLATSHPHYDEAGNVLNMGTSIVEKGKTKYVIFKIPATVPEGKKQGKSPWKHTEVFCSIPSRSLLSPSYYHSFGVTENYVIFLEQPFRLDILKMATAYIRRMSWASCLAFHREEKTYIHIIDQRTRQPVQTKFYTDAMVVFHHVNAYEEDGCIVFDVIAYEDNSLYQLFYLANLNQDFKENSRLTSVPTLRRFAVPLHVDKNAEVGTNLIKVASTTATALKEEDGQVYCQPEFLYEGLELPRVNYAHNGKQYRYVFATGVQWSPIPTKIIKYDILTKSSLKWREDDCWPAEPLFVPAPGAKDEDDGVILSAIVSTDPQKLPFLLILDAKSFTELARASVDVDMHMDLHGLFITDMDWDTKKQAASEEQRDRASDCHGAPLT

SEQ ID NO: 13 - protein, 360 aa, unidentified
  REGION 1..360: note = Sc.ADH6; Aldehyde dehydrogenase
MSYPEKFEGIAIQSHEDWKNPKKTKYDPKPFYDHDIDIKIEACGVCGSDIHCAAGHWGNMKMPLVVGHEIVGKVVKLGPKSNSGLKVGQRVGVGAQVFSCLECDRCKNDNEPYCTKFVTTYSQPYEDGYVSQGGYANYVRVHEHFVVPIPENIPSHLAAPLLCGGLTVYSPLVRNGCGPGKKVGIVGLGGIGSMGTLISKAMGAETYVISRSSRKREDAMKMGADHYIATLEEGDWGEKYFDTFDLIVVCASSLTDIDFNIMPKAMKVGGRIVSISIPEQHEMLSLKPYGLKAVSISYSALGSIKELNQLLKLVSEKDIKIWVETLPVGEAGVHEAFERMEKGDVRYRFTLVGYDKEFSD

SEQ ID NO: 14 - protein, 361 aa, Vanderwaltozyma polyspora
  REGION 1..361: note = misc_feature - Vanderwaltozyma polyspora, A7TLS3
MSYPEYFQGIGVVDKDNWTHPKHVSYAAKEFKECDVDIEIEACGVCGSDIHCASGDWGERRDKLVVGHEVIGRVIKLGPECHTGLKIGDRVGVGAQVASCLDCERCEHDNEPYCPKFVTTYNQAYPDGYISQGGYASHIRVHEHFVIPVPEKIPTHLAAPLLCGGITVYSPLVRGGCAPGKKVGIVGIGGIGHMGILFAKAMGAEVYAFSRHENKREDSMKLGADHYIATLEEEGWTEKYFNSLDVMILCASSLSQINLDDLIKVMKVEGKIISICVPENDEMMIVKPLGLLGVYVSNSLLGSRKELLELLHMVAEKDIKIWVEKLPVGEAGVSEAFIRMKKGDVRYRFTLTDFDKEFPKN

SEQ ID NO: 15 - protein, 360 aa, Vanderwaltozyma polyspora
  REGION 1..360: note = misc_feature - Vanderwaltozyma polyspora, A7TM83
MAYPEMFEGIGVTNPEDWKHPKKVTYEPKKFDDQDVDIKIEACGVCGSDIHCAASHWGPVVKNQVVGHEIVGRVVKVGPKCTTGIKIGDRVGVGAQVASCLECSRCQNDNEPYCPRFVTTYGQPYPDGYVSQGGYASHVRVHEHFAIPVPEELPLHLAAPLLCGGITVYSPLLRGGCGPGKKVGIVGIGGIGHMGILFAKAMGAEVYAFSRSHSKEKDAKELGADHYIATLEDKDWTEKYFDTLDLIVVCASSLTDVNFDELIKVMKVNTKILSISVPSIDETLTLKPFGLLGVTIGNSALGSRKEIEQLLKLVVDKDIKIWVEQLQVGEAGVAEAFERMDKGQVRYRFTLMGFDKEFGN

SEQ ID NO: 16 - protein, 360 aa, Vanderwaltozyma polyspora
  REGION 1..360: note = misc_feature - Vanderwaltozyma polyspora, A7TPJ1
MSYPEKFQGIGITNREDWKHPKKVTFEPKQFNDKDVDIKIEACGVCGSDVHCAASHWGPVAEKQVVGHEIIGRVLKVGPKCTTGIKVGDRVGVGAQAWSCLECSRCKSDNESYCPKSVWTYSIPYIDGYVSQGGYASHIRLHEHFAIPIPDKLSNELAAPLLCGGITVYSPLLRNGCGPGKKVGIVGIGGIGHMGLLFAKGMGAEVYAFSRTHSKEADAKKLGADHFIATLEDKDWTTKYFDTLDLLVICASSLTDINFDELTKIMKVNTKIISISAPAADEVLTLKPFGLIGVTIGNSAIGSRREIEHLLNFVAEKDIKPWVETLPVGEAGVNEAFERMDKGDVKYRFTLVDFDKEFGN

SEQ ID NO: 17 - protein, 361 aa, Clavispora lusitaniae
  REGION 1..361: note = misc_feature - Clavispora lusitaniae, C4Y4W4
MAYPETFEAIGVVDYDDWKNPKRFEYKPQEFRDYDVDIEIDACGVCGSDLHAASGNWGRPYAPVAVGHEIIGRVVKVGPKAKEGLKVGDRVGVGAQCDCDNTCAACLSKSEPNCRNHVGTYFGHNKETGFNTIGGNASHIRVNSQFVFKIPDSLKTEHAAPLLCGGITGFAPLLQNNVGKGTRVGVVGIGGIGHMTILFAKALGAEVTAISRTDSKKEMAKKLGADHYAATADEDFSKYADTLDLIVNTGSSFSGSSMDKILSMLRARGKFVFITAPPATEKLELTPFQLLANNTSIQGSLLGTPEEIQYMLNFAAEHKIEPWVETIDMSEENLATAWKRAEEGDVRFRFTMVNYDKCFKR

SEQ ID NO: 18 - protein, 362 aa, Clavispora lusitaniae
  REGION 1..362: note = misc_feature - Clavispora lusitaniae, C4Y4W5
MVYPETFKAVGVVDFDDWLHPKSFEYKPQEFRDYDVDIEIEACGVCGSDIHAANGDWGRPYAPVAVGHEIIGKVVKVGPKAKEGIKVGDRVGVGAQCDCDNTCIACKKNLQSNCRNHVGTYFGVNKETGFKTIGGNASHIRVNSQFVFNIPDSLKTEHAAPLLCGGITGFSPLLQHNVGKGTKVGVVGIGGIGHMTILFAKALGAEVTAISRTDSKKEVAKQLGADHFAATADEDFIEKYADTLDLIVNTGSSFSGSSLDKLLSMLVPRGKFVFITAPPVGENIELSPMKLLFNNSSIQGSAIGSPEEIQYMLNFAAEHKIEPWVETIDISEENLGKAWKRAEEGDVKFRFTMVGYDKFFKN

SEQ ID NO: 19 - protein, 363 aa, Clavispora lusitaniae
  REGION 1..363: note = misc_feature - Clavispora lusitaniae, C4YAF2
MVYPETFQALGVVDYDDWLHPKTFEYKPQEFRDYDIDIEIEACGVCGSDIHAASGNWGRPYAPVAVGHEIVGKVVKVGPKAKSGIELGDRVGVGAQCDCDNSCIACELNLQSNCRNHVGTYFGHNKDTGFNTIGGNASHIRVNSQFAFKIPPSLKTEHVAPLLCGGITGFSPLLQHKVGKGTKVGVVGIGGIGHMTILFAKALGAEVTAISRTDSKKDLAEKLGADHYAATSDEEFEKKYSDTLDIIVNTGSSFAGSAVEKLLKMLRPRGKFVFITVPPSGENLELSPVQLVSNNASVQGSSTGSLEEIQFMLDFAAEHNIQPWVETIDISEENLGKAWERAQKGDVKFRFTMVGYDKFFKKK

SEQ ID NO: 20 - protein, 360 aa, Eremothecium cymbalariae
  REGION 1..360: note = misc_feature - Eremothecium cymbalariae, G8JME0
MGELKEITGIAVVEGTEWTSPKKVSFKPKTVYARDIVLKVEACGVCGSDIHCAAGHWGPRNNPLVVGHEIIGRVIEVGSGCKEGLKVGDRVGVGAQALSCLECSRCESENEQYCSNMTGTYNFPYPDGYISQGGYASHVIVHEHFAIPIPDNIPSHLAAPLMCGGITAFSPLLRSNVGPGTSVGVVGIGGIGHMAVIFAKALGANVTAISRRSNKKDDALKLGADVYIAAVEDKDWHKTYANTLDLIVVCSSSLTELDFDRIPSLLKVGGKVLSIAIPEASERLVLRPLLLKGISIGTSVIGSPAEIKKLLDFVSKKNLKIWVEEVPISEAGVAEVFRRMNSGDVRYRFSLVDFDKEFGA

SEQ ID NO: 21 - protein, 366 aa, Kluyveromyces lactis
  REGION 1..366: note = misc_feature - Kluyveromyces lactis, Q6CMI8
MSYPDNFEGFAVVDSHKKWLSPEKVVYPAKQFNPRDVDIEIEACGVCGSDVHCAKGNWGEMKLPLVVGHEIIGKVVRLGDKCDTGLKVGDRVGVGAQVSACLDCNRCNNDNEQYCPKFVTTYSQPYFEDGYVGQGGYASHIRVHEHFVIPIPESLETNNVAPLLCGGATVYSPLKRNGCGPGKKVGIVGIGGIGHMGILLAKAMGAEVYAISRSHAKEETSKQLGADHYIATEDEGWETKYFDTLDFMVLCGNSLSAVDFGKLPKILKAQGVIASISVPDQHEAIHLHPAQLLGFQITQSCLASRAEIIEMLNLVAEKKIKLWVETVPISEENISTVFKRMESGDVRFRFTLTDFDKQFDQFKAKK

SEQ ID NO: 22 - protein, 320 aa, Danio rerio
  REGION 1..320: note = misc_feature - Danio rerio, B8A539
MMLALVAFAAGLGLVALILRLLSPQIRKYAAGGSCRSTVRLDGKVALVTGANSGIGKETALDLASRGARVILACRDLEKAEEAAAEIRTRVGGAKVEVRELDLADCCSIRAFAQRFLREVDHLHILINNAGVMMCPYMKTADGFEMQIGVNHLGHYLLTYLLIGLLKRSAPSRIVVVSSLAHNFGWIRFHDLHSQGSYNSGLAYCQSKLANVLFTRELARRLQGSNVTVNSVHPGTVRSELVRHSTLMSLLFAFFSMFLKSPKEGAQTSIYCAVAEELQSISGKHFSDCAPAFVAPQGRSEETARKLWDVSCELLGIEWD

SEQ ID NO: 23 - protein, 318 aa, Gasterosteus aculeatus
  REGION 1..318: note = misc_feature - Gasterosteus aculeatus, G3PFC0
MLLLLIVAGLGVVTLLVILFAPHIRKYAAGGVCSSTASLDGKVALITGANTGIGKETALELAVRGARVIMACRNVDKGEEAADSIRAAHPEAQVEVRELDLADTCSIRAFAQKFLGEVNSLHILINNAGVMMCPYTKTIDGFEMHIGVNHLGHFLLTSLLIGLLKRSAPARIVVVSSLAHNFGWIRFHDLHSQGSYNSGLAYCQSKLANVLFTRECARKLKGTDVTVNSVHPGTVNSDLTRHSTLMTIFFTVFSTFLKTPREGAQTSIYCAVADELHSISGKHFSDCAPAFVAPQGRSEDTARRLWAASCELLGIEWD

SEQ ID NO: 24 - protein, 318 aa, Latimeria chalumnae
  REGION 1..318: note = misc_feature - Latimeria chalumnae, H3BEA6
MCFWMFTGISALVGFLLSVLRPYIRRFAAGGVCKSNARLDGKTVLITGANTGVGKETMRDLARRGARVIAACRDVSKGEAAVNEIRMDTGNEQIVVRKLDLADTRSIREFADNLLREEKALHILINNAGVMMCPYSKTEDGFEMQLGVNHLGHFYLTYLLLNLLKHSAPARIINLSSLAHNLGWIKFADLQSEKSYNSGIAYCQSKLANVLFTRELARRLQGTKVTANSVHPGTVHSELTRHSFMVSLAWKIFSVFIKTPMEGAQTSIYCAVAEELESISGKYFSDCSPAFVAPQGRNDETAKRLWDISCKLLAIKWD

SEQ ID NO: 25 - protein, 318 aa, Tetraodon nigroviridis
  REGION 1..318: note = misc_feature - Tetraodon nigroviridis, H3DMU7
MLLLLIVAGLGVVTLLVVLFKPQIRKYAAGGVCQSTATLYAKTVLITGANTGIGKETALDLATRGARVIMACRDVDKGEEAAASIRAACPKATVEVRELDLADTCSIRAFAQKFLREVNQLHILINNAGVMMCPYTKTIDGFEMHIGVNHLGHFLLTHLLIGLLKRSAPARIVVVSSLAHNFGWIRFHDLHSQGSYNSGLAYCQSKLANVLFARELARRLKGTEVTVNSVHPGTVNSDLTRHSTLMTIFFTIFAMFLKTPQEGAQTSIYCALAEELHSISGKHFSDCAPAFVAPQGRSEETARRLWQASCELLGIEWD

SEQ ID NO: 26 - protein, 319 aa, Oreochromis niloticus
  REGION 1..319: note = misc_feature - Oreochromis niloticus, I3KUB2
MMFLLLIIAGLGVVTLLVILFAPQIRKYAAGGVCKSTTRLDGKTVLITGANTGIGKETALDLAMRGARVIMACRDVEKGEEAAASIRASYPEARVEVRELDLADTCSIRAFAQKFLREVNQLHILINNAGVMMCPYTKTVDGFEMHIGVNHLGHFLLTSLLIGLLKRSAPARIVVVSSLAHNFGWIRFHDLHSQGSYNSGLAYCQSKLANVLFTRELASRLKGTNVTVNSVHPGTVNSDLTRHSTLMTILFTIFSVFLKTPREGAQTSIYCAIAEELHSISGKHFSDCAPAFVAPQGRSAETARRLWDVSCELLGIEWD

SEQ ID NO: 27 - protein, 318 aa, Xiphophorus maculatus
  REGION 1..318: note = misc_feature - Xiphophorus maculatus, M4AK03
MFVLLIIAGLGVVTLLVILFAPHIRRYAAGAVCKSTARLDGKTVLITGANTGIGKETALDLAVRGARVIMACRNVEKGEEAAASIRAAYSEAKVEVRELDLADTCSIRAFAQKFLREVNHLHILINNAGVMMCPYTKTVDGFEMHIGVNHLGHFLLTYLLIGLLRRSAPARVLVVSSLAHNFGWIRFHDLHSQGSYNSGLAYCQSKLANVLFARELARRLKGTNVTVNSVHPGTVNSDLTRHSTLMMILFTVFSVFLKTPLEGAQTSVYCAVAEELHSVSGKHFSDCAPAFVAPQGRSEETASKLWDVSCELLGIDWD

SEQ ID NO: 28 - protein, 318 aa, Anolis carolinensis
  REGION 1..318: note = misc_feature - Anolis carolinensis, R4GAG6
MQEWLGGALALGLALLWLLAAPSIRKYISGGKCTSTAKLNGKVVVITGANTGIGKETARDLAGRGARVILACRDMEKAEAAANEIRTKTGNQQVIAKKLDLADTKSIREFAENFLKEEKELHILINNAGVLLCPYSKTVDGFEMQFAVNHFGPFLLTFLLIERMKESAPSRIVNVSSLAHCLARIRFEDLQGEKSYHRGLAYCNSKLASILFTRELARRLQGTRVTANALHPGSIVSELGRHLTILIFLGKLLTFFLKTPQEGAQTSVYCAVAEELESVSGKYFSDCKPAYVWPQGCDDETAKKLWDVSCELLGIQWD

SEQ ID NO: 29 - protein, 322 aa, Anolis carolinensis
  REGION 1..322: note = misc_feature - Anolis carolinensis, R4GC18
MQDFPGCALALGLALALPLLGLLAAPYIRKYFAGGKCTSTAKLNGKVVVITGANTGIGKETAKDLAGRGARVILACRDMVKAEAAASEIRTKTGNQQVIAKKLDLADTKSIREFAENFLEEEKELHILINNAGVMMCPYSKTADGFEMHFGVNHLGHFLLTFLLTECLKKSAPSRIVNVSSLAHHGGRIRFEDLQGEKSYQWGLAYCHSKLAGILFTRELARRLQGTGVTVNALHPGTVASDLPRHSTIMNFLWKLLPFLLKTPQEGAQTSVYCAVAEELGSVSGKYFSDCKPAYVSPQGRDDETAKKLWDVSCELLGIQWN

SEQ ID NO: 30 - protein, 317 aa, Fundulus heteroclitus
  REGION 1..317: note = misc_feature - Fundulus heteroclitus, A0A146ZLA9
MVSPEQKVVLITGCSSGIGLRMAVMLAKDEKKRYHVIATMRDLKRKDKLVEAAGDAYGKTLSLAVLDVCHDESVKQCIDSIKDRHVDVLINNAGIGLVGPIESIPIEEMKKVFETNFFGVIRMIKEVMPDMKKRRAGHIIVVSSVMGLQGVVFNDVYAASKFAMEGFCESLAVQLLKFNVILSMIEPGPVHTEFEAKMIQDVKQKEYPGADPDTVHYFKNVYLPSSVDIFEALGQTPDDIARCTKKVIEASRPRFRNLTNPLYTPIVALKYADETGGLSVHAFYHMLFNMGPLMHVSMTAMKYLTCGCLRRRTISPN

SEQ ID NO: 31 - protein, 316 aa, Xenopus laevis
  REGION 1..316: note = misc_feature - Xenopus laevis, A0A1L8GVT0
MAGDSKKTVLVTGCSSGIGLKIAVQLAKDPQERYYVIATMRDLGKSGKLEAEVGEALNKTLVIRQLDVTKEDSVKKCLDDLPGQRVDILVNNAGVGQIGPIESFSMEDMRKVFETNFFGVVHLIKKVLPGMKKRGGGHIVVISSIMGLQGVIFNDIYAASKFAIEGFCECLAVQLLKFNIFVSMIEPGPVNTEFEMKLMEEVSRSDFPGSDADTIRYFKDIYLPSAHEIFSSMGQTPEAVAKATVKVIGMQQPVFRHQTNVLYTPLTALKYADNTGNLSVRTFYNLLFNYGTLFHLSLGFLRCITCNCFRRRVMPV

SEQ ID NO: 32 - protein, 316 aa, Xenopus laevis
  REGION 1..316: note = misc_feature - Xenopus laevis, A0A1L8H2R2
MAGDSKKTVLITGCSSGIGLQIAAQLAKDPQERYYVIATMRDLGKSGKLEAEVGEFLNKTLVIRQLDVTKEDSVKHCLDALPGQRIDILVNNAGVGQIGPIESLSMEDMRKVFETNFFGVVHLIKAVLPGMKKRGSGHIVVISSVVGLQGVIFNDIYSASKFAIEGFCECLAVQLLKFNIFVSMIEPGPVNTEFEMKLMEEVSRSDFPGADADTIRYFKDIYLPSAHEIFSSIGQTPEAVAEATVKVIGMRQPVFRHQTNVLYTPLTAMKYADNTGNLSIRTFYNLLFNYGTLFHFSLGFLRCITCNCFRRRVMPV

SEQ ID NO: 33 - protein, 317 aa, Scleropages formosus
  REGION 1..317: note = misc_feature - Scleropages formosus, A0A1W5A3B1
MASDGQKVVLITGCSSGIGLRVAVLLAKDDRKRYHVIATMRDTKRKDKLLEAAGDTYGKTLTLQTLDVCSEESVKQCINSIKDRRVDILINNAGIGLLGPVESISIEEMKKVFETNFFGMVRMIKEVMPDMKMRRSGHIIVMSSVMGLQGVVFNDVYTASKFAMEGFCESLAVQLLKFNVNVSMIEPGPVHTEFEVKMMQDVAKMEYPGADADTVRYFKDVYLPSSLDIFEAMGQSPEDIARCIKKVIEARRPRFRNLTNSLYTPIVAMKYADETGGLSVHTFYNLLFNFGPLMHITMNILKCLTCSCLRRRTISPN

SEQ ID NO: 34 - protein, 318 aa, Cyprinus carpio
  REGION 1..318: note = misc_feature - Cyprinus carpio, B5UAY7
MASGGEQKVVLITGCSSGIGLRIAVLLARDELKRYHVIATMRDLRKKDRLVEAAGDTYGQTLTLLSLDVCSDDSVRKCIDSVKDRHIDILINNAGVGLLGPVESISMDEMKKVFETNFFGTVRMIKEVMPDMKKRRAGHIIVMSSVMGLQGVVFNDVYTASKFAIEGFCESMAVQLLKFNVKLSLIEPGPVHTEFETKMMEEVAKMEYPGADADTVRYFKDVYIPSSIDIFEALGQTPEDIAKCTKKVIESSRPRFRNLTNSLYTPIVAMKYADESGGLSVHTFYNLLFNFGSLMHITMSILKCLTCSCLRRRTISPD

SEQ ID NO: 35 - protein, 310 aa, Danio rerio
  REGION 1..310: note = misc_feature - Danio rerio, E7F0C2
MGTRKVLVTGCSSGIGLAVAVRLAKDELRRFKVVATMRDLDRREALERAAGETLNRSLEIRQLDATCEDSIRDCVNSLPDRQVDVLVNNAGVGMIGPLECQSMSSMQDLFNTNFFGLVRLVKELLPQMKKRQSGHIIVMSSVLGIQGLLFNDLYAASKFAVEGFCESLAVQAMKFNVKMTLVEPGPVVTEFERKVYEEAETMDLSETDEETAQIFRQIYLPYSRRVFHSIGQTPEEVAEQTTRLILSKNPPFRHQTNRLYMPLTAMKHADPTGRLPIDAFYKMIFQHDRIFSASLNIMRILNRKIGKSAA

SEQ ID NO: 36 - protein, 317 aa, Gasterosteus aculeatus
  REGION 1..317: note = misc_feature - Gasterosteus aculeatus, G3P670
MANSGQKVVLITGCSSGIGLRIAVTLAKDEKKRYHVIATMRDLKKKDKLLEAAGDVYGKTLMLLPLDVCSDESVKQCINNVKDRHIDILINNAGVGLLGPLESISIEEMKRVFETNFFGVVRMIKEVMPDMKKRRSGHIVVMSSVMGLQGVVFNDVYTASKFAMEGFCESMAVQLMKFNIRLSMIEPGPVHTEFETKMMEDVAKMEYPGVDADTVRYFKDVYLPSSTDIFEAMGQTPEDIAKCTKKVIESNSPRFRNLTNSLYTPIVALKYADETGGLSVNTFYNLLFNFGPLMHITMSILKCLTCSCLRRRTVSPN

SEQ ID NO: 37 - protein, 317 aa, Gasterosteus aculeatus
  REGION 1..317: note = misc_feature - Gasterosteus aculeatus, G3PW42
MASPGQKVVLITGCSSGIGLRMAVMLAKDQQQRYHVIATMRDLKRKDKLVEAAGDAYGKTLSLAVLDVCTDESVKRCIDGIKDRHVDVLVNNAGIGLVGPIESIPIDEMKKVFETNFFGVIRMIKEVMPDMKRRRGGRIVVVSSVMGLQGVVFNDVYAASKFAMEGFCESLAVQLLKFNVTLSMIEPGPVHTEFEAKMIQDVKQKEFPGADADTVNYFKNVYLPSSVDIFETLGQTPDDIARVTKKVIEASSPRFRNLTNPLYTPIVALKYADETGGLSVHAFYHMLFNLGPLMHVSMTAMKYLTCGCLRSRTVAPN

SEQ ID NO: 38 - protein, 320 aa, Oryzias latipes
  REGION 1..320: note = misc_feature - Oryzias latipes, H2LMU2
MANSGQKVVLITGCSSGIGLRIAVTLASDEKKRYHVIATMRDLKKKDKLLEAAGDVYGKTLMLLPLDVCSDESVRQCINSVKDRHIDILINNAGVGLLGPVESISIEEMKRVFDTNFFGVVRMIKEVMPDMKKRRSGHIVVMSSVMGLQGVVFNDVYTASKFAMEGFCESMAVQLLKFNIRLSMIEPGPVHTEFETKMMEEVAKMEYPGVDADTIRYFKDVYLPSSIDIFEAMGQTPEDIAKCTKKVIESDSPRFRNLTNSLYTPIVALKYADETGGLSVNTFYNLLFNFGPLMHITMNILKCLTNRTIKRYQSTKTEPS

SEQ ID NO: 39 - protein, 317 aa, Oryzias latipes
  REGION 1..317: note = misc_feature - Oryzias latipes, H2M0R5
MVSPGQKVVLITGCSSGIGLRIAVMLAKDEKKRYHVIATMRDLKRKDKLVEAAGDTYGKTLSLAVLDVCSDESVKQCINGIENRHVDILINNAGIGLVGPLESISIEEMKKVFETNFFGVVRMIKEVMPDMKKRKDGRIIVVSSVMGLQGVVFNDVYSASKFAMEGFCESLAVQLLKFNVIVSMIEPGPVHTEFEVKMIQEVKQRDFPGADAETVRLFKEVYLPSSVDIFEALGQTPDDIARCTKKVIEASSPRFRNLTNALYTPIVAMKYADETGTMSVHAFYHLLFNLGPLMHVSVMAMKYLTCGCLRRRTISPN

SEQ ID NO: 40 - protein, 317 aa, Fugu rubripes
  REGION 1..317: note = misc_feature - Fugu rubripes, H2UL76
MASPGQKVVLITGCSSGIGLRMAVMLAKDPQKRFHVIATMRDLKRKDKLLEAAGDTYEKTLSLAVLDVCSDESVKQCINGIKDRHVDVLINNAGIGLVGPVESISIEEMKKVFETNFFGVIRMIKEVMPDMKKRRGGHIIVVSSVMGLQGVVFNDVYAASKFAMEGFCESLAVQLFKFNVNLSLIEPGPVHTEFEAKMMQDVRQKDFPGTDPETMHYFKNVYLPSAIDIFEMFGQTPDDIARCTVKVVESSRPRFRNLTNPLYSPIVALKYADDTGNLSVRAFYHMLFNMGSVMHVSMTVMKYLTCGCLRSRTISPN

SEQ ID NO: 41 - protein, 317 aa, Takifugu rubripes
  REGION 1..317: note = misc_feature - Takifugu rubripes, H2UZY9
MTQKVVLITGCSSGIGLSLAARIAKDEKKRFMVYATMRNLSKGEALVKAAGRALGRTLEIKQLDVCDEDSIRACVDSLPDRRVDILISNAGMGLIGPIECQSIGEMKTVMDTNFFGLVRLVKEILPDMKRRKRGHIVVISSVMGIQGILFNDIYAASKFAVEGFCESLAVQALRFQLNISLIEPGPVITEFERKVYEEGLKTDLSKADEVTADMFTNIYLKNYKQIFETLGQTADDIAEHTLKIITMDNPPFRHQTNTLYTPMTTLKYADPNGDLPIDTFYKMVFEHDKIFNASLNFLKLLRWRSRKSFTLEKDNNN

SEQ ID NO: 42 - protein, 308 aa, Anolis carolinensis
  REGION 1..308: note = misc_feature - Anolis carolinensis, H9GCI2
MAVRNVLVTGCSSGIGLALAVRLARDELKRFRVIATMRNLAKRQALEMQAGPVLEKTLEIKELDVTSEESIRHCVDSIPQRRVDVLVNNAGLGMIGPVESQSLTSMQHLFDANFFGLVRLVKEVFPDMKKRKQGHVVVMSSVLGLQGLLFNDIYSASKFAVEGFCESLALQALKFGVNISLIEPGPVITEFERKLYEEAANMDLSGLDKETLDIFQGFYMAYSKEVFTALGQSPEEVAEHTMKVITSDQPPFRYQTNSLYTPITTLKHADPSGNLPLNAFHQMIFQHDRLFKASLSLLKMLQWRKRRD

SEQ ID NO: 43 - protein, 316 aa, Anolis carolinensis
  REGION 1..316: note = misc_feature - Anolis carolinensis, H9GI25
MAKATPRTVLITGCSSGIGLRMAVQLAQDPGKRFHVIATMRDLRKKDKLEAAAGDTLNKTLTIQRLDVCNDESVTECINSLPGKQLDVLVNNAGVGLVGPVESISIDDMKRVFETNFFGAIRMIKAVLPEMKKRQKGHIVVISSVMGLQGVPFNDVYAASKFAMEGFCESLAVQLLKFNIFVSMVEPGPVNTEFEMKLMEEVARSEFPGADAATVRYFKEVYLPASHEIFTTMGQTPESVAKAVVNVIAKERPPFRTQTNTLYTPLVALKYADTSGDLSVGTYYNLLFRFTGLFHLSMSCLKCITCSCFRRRVTPA

SEQ ID NO: 44 - protein, 317 aa, Oreochromis niloticus
  REGION 1..317: note = misc_feature - Oreochromis niloticus, I3IZM1
MANSGQKVVLITGCSSGIGLRIAVILAKDEKSRYHVIATMRDLKKKDKLVEAAGDAYGKTLTLLPLDVCSDESVRQCVNNVKDRHIDILINNAGVGLLGPVESISIEEMKKVFETNFFGVVRMIKEVMPDMKKRRSGHIIVMSSVMGLQGVVFNDVYTASKFAMEGFCESLAVQLMKFNIRLSMIEPGPVHTEFETKMMEDVAKMEYPGADADTVRYFKDVYLPSSIDIFEAMGQTPDDIAKCTKKVIESNNPRFRNLTNNLYTPIVALKYADETGGLSVNTFYNLLFNFGPLMHITMSILKCLTCSCLRRRTISPN

SEQ ID NO: 45 - protein, 325 aa, Oreochromis niloticus
  REGION 1..325: note = misc_feature - Oreochromis niloticus, I3IZP7
MATRRVLVTGCSSGIGLAVAARLAKDELRRFKVVATMRDLGKRGPLEKAAGDSLNKTLEIKELDACCETSIKECINSLPNRRVDVLVNNAGVGMIGPLECQSIDAMKELFDTNFFGLVRLVKEVLPDMKQRQSGHIVVMSSVMGIQGLLFNDVYSASKFAVEGFCESLAVQAMKFNIKTTLVEPGPVVTEFERKMYEGAEKMDLSGTDEETARIFRELYLPYSKEVFTSLSQTPEEVAQQTVNVITAKDPPLRHQTNKVYMPMTALKHADPTGRLPLDTFYKMIFKHDRVFSATLGVLRLLQRRAEQIKAIYSFFLSKSVYHFIS

SEQ ID NO: 46 - protein, 308 aa, Xiphophorus maculatus
  REGION 1..308: note = misc_feature - Xiphophorus maculatus, M3ZJL7
MGTRRVLVTGCSSGIGLAVAVRLAKDETKRFKVVATMRDLEKRGPLEKAAGDLLNKTLVIQKLDACCEESIKECINSIPNRQVDVLVNNAGIGMIGPLECQSITSMKELFEVNFFGLVRLVKEVLPDMKQRQSGHIVVMSSVMGIQGLLFNDVYSASKFAVEGFCESLAVQAMKFNIKTTLVEPGPVMTEFERKVYEDAEIIDLSETDEETARIFRDIYLPYSRKVFTTLGQTPEDVAEQTQKVITAKEPPLRHQTNRLYMPMTALKHADPTGRLPLEAFYKMIFKHDTVFNATLGILRMMNRRKGNK

SEQ ID NO: 47 - protein, 317 aa, Xiphophorus maculatus
  REGION 1..317: note = misc_feature - Xiphophorus maculatus, M3ZLM4
MNQKVVLITGCSSGIGLALAARIAKDEKKRFMVYATMRNLGNAEPLVEAADRTLGRTLELKQLDVCSEESIKACVDSLPERRVDILISNAGMGLIGPIECQSIDEMKTVMDTNFFGLVRLLKEILPDMKKRKKGHIVVISSVMGIQGILFNDVYAASKFAVEGFCESLAVQALRFNLNISLIEPGPVITEFERKVYEEGLKTDLSKADKVTADMFTNIYLKNYKQIFESLGQTAEDVAEHTHRIITMDDPPFRHQTNTLYTPMTTLKYADPNGDLPIETFYKMVFEHDKVFNASLNFLKLLRWRSRKSFTLEKDKST

SEQ ID NO: 48 - protein, 317 aa, Xiphophorus maculatus
  REGION 1..317: note = misc_feature - Xiphophorus maculatus, M4AEE7
MVSPEQKVVLITGCSSGIGLRMAVMLAKDEKKRYHVIATMRDLKRKDKLVEAAGDAYGKTLSLAVLDVCSDESVKQCINGIKDRHVDVLINNAGIGLVGPVESISIEEMKKVFETNFFGVIRMIKEVMPDMKQRRGGHIIVVSSVMGLQGIVFNDVYAASKFAMEGFCESLAVQLLKFNVILSLIEPGPVHTEFEAKMIQDVKQKEYPGTDPDTLHYFKNIYLPSSVDIFEAFGQTPDDIAKCTKKVIEASRPRFRNLTNPLYTPIVALKYADETGGLSVHAFYHMLFNMGPVMHVSVTAMKYLTCGCLRRRTISPN

SEQ ID NO: 49 - protein, 317 aa, Xiphophorus maculatus
  REGION 1..317: note = misc_feature - Xiphophorus maculatus, M4ATJ1
MANSGQKVVLITGCSSGIGLRIAVTLAKDEKKRYHVIATMRDLKKKDKLLEAAGDTYGKTLMLLPLDVCSDESVKECINNIKDRHIDILINNAGVGLLGPVESISIDEMKRVFDTNFFGVVRMIKEVMPDMKKRRSGHIVVMSSVMGLQGVVFNDVYTASKFAMEGFCESMAVQLMKFNIRLSMIEPGPVHTEFETKMMEDVAKMDFPGVDADTVRYFKDVYLPSSIDIFEAMGQTPEDIAKCTKMVIESERPRFRNLTNSLYTPIVALKYADETGGLSVNTFYNLLFNFGPLMHITMSILKCLTCSCLRRRTISPN

SEQ ID NO: 50 - protein, 318 aa, Danio rerio
  REGION 1..318: note = misc_feature - Danio rerio, Q6PC70
MASGGGQKVVLITGCSSGIGLRIAVLLARDEQKRYHVIATMRDLKKKDRLVEAAGEVYGQTLTLLPLDICSDESVRQCVNSVKDRHIDVLINNAGVGLLGPVESISMDEMKRVFETNFFGTVRMIKEVMPDMKKRQAGHIIIMSSVMGLQGVVFNDVYTASKFAIEGFCESMAVQLLKFNVKLSLIEPGPVHTEFETKMMEEVAKMEYPGADPDTVRYFKDVYVPSSIDIFEAMGQTPDDIAKCTKKVIETSQPRFRNLTNSLYTPIVAMKYADETGGLSVQTFYNLLFNFGSLMHISMSILKCLTCNCLRRRTISPD

SEQ ID NO: 51 - protein, 318 aa, Danio rerio
  REGION 1..318: note = misc_feature - Danio rerio, Q7T2I8
MASGGGQKVVLITGCSSGIGLRIAVLLARDEQKRYHVIATMRDLKKKDRLVEAAGEVYGQTLTLLPLDICSDESVRQCVNSVKDRHIDVLINNAGVGLLGPVESISMDEMKRVFETNFFGTVRMIKEVMPDMKKRQAGHIIVMSSVMGLQGVVFNDVYTASKFAIEGFCESMAVQLLKFNVKLSLIEPGPVHTEFETKMMEEVAKMEYPGADPDTVRYFKDVYVPSSIDIFEAMGQTPDDIAKCTKKVIETSQPRFRNLTNSLYTPIVAMKYADETGGLSVQTFYNLLFNFGSLMHISMSILKCLTCNCLRRRTISPD

SEQ ID NO: 52 - protein, 317 aa, Gadus morhua
  REGION 1..317: note = misc_feature - Gadus morhua, UPI00023F1C4D
MANSGQKVVLITGCSSGIGLRIAVTLAKDEKKRYHVIATMRDLKKKEKLVEAAGDTFGKSLTLHPLDVCSDDSVSQCINSVKDRHIDILINNAGVGILGPVESISIDQMKKVFETNFFGVVRMIKEVMPDMKARRSGHIIVMSSVMGLQGVVFNDVYTASKFAMEGFCECMAVQLMKFSVRLSMIEPGPVHTEFETKMMADVAKMEFPGVDADTVRYFKDVYLPSSIDIFEAMGQTPEDIAKCTKKVIESSSPRFRNLTNSLYTPIVALKYADETGGLSVNTFYNLLFNLGPLMHITMSILKCLTCSCLRRRTVSPN

SEQ ID NO: 53 - protein, 317 aa, Gadus morhua
  REGION 1..317: note = misc_feature - Gadus morhua, UPI00023F3DEC
MASNGQKVVLITGCSSGIGLRMAVMLANDEQKRYHVIATMRDLKRKDKLVEAAGEAYGKTLSLAVLDVCTDESVKQCIDGIKDRHVDVLINNAGIGLVGPLESIPMEEMKKVFETNFFGVIRMIKEVMPDMKKRRGGHIIVISSVMGLQGVVFNDVYAASKFATEGFCECLAVQLLKFNVTLSMIEPGPVHTEFEAKMIQDVRQKEYPGTDPETVNYFKNVYLPSSVDIFETLGQTPDDIARVTKKVIEASSPRFRNLTNPLYTPIVALKYADETGGLSVHAFYHMLFNLGPLMHVSMKAMQYLTCGCLRSRTISPN

SEQ ID NO: 54 - protein, 316 aa, Xenopus laevis; identical to SEQ ID NO: 31
  REGION 1..316: note = misc_feature - Xenopus laevis, UPI00084DD5CA

SEQ ID NO: 55 - protein, 696 aa, Gibberella fujikuroi
  REGION 1..696: note = misc_feature - Gibberella fujikuroi, Q5GN50
MKFLQQNSFTQTSMSQPHEDVSPAIRHPYLTGNFAPIHKTTNLTPCTYSGCIPPELTGGQYVRNGGNPVSHQDLGKDAHWFDGDGMLSGVAFRKASIDGKTIPEFVNQYILTDLYLSRKTTSIASPIMPSITTLVNPLSTMFQIMFATFRTIFLVILSNLPGSQQAIKRISVANTAVLYHDGRALATCESGPPMRIQLPSLDTVGWFDGVEAEGEPEISQAGSDDSPFGGSGIFSFMKEWTTGHPKVDPVTGEMLLYHNTFMPPYVHCSVLPKSNEKAPGHRLVNQPVLGVSGARMMHDFGASRSHTIIMDLPLSLDPLNTMKGKEVVAYDPTKPSRFGVFPRHLPSSVRWFHTAPCCIFHTANTWDSQSSEGELSVNLLACRMTSSTLVYTAGNIRPPVRSRCTQARVWSDEREETACRYKEAPALESPGESTGLADYFPITAESDDYDQCRLYYYEFDLAMESRNHVKSQWALSAIPFEFPSVRPDREMQEARYIYGCSTSTSCFGVALGRADKVDLLVKMDAKTLIQRGKKMNATSITGCVDRRSVCEILQEQRKDDPIYIFRLPPNHYAQEPRFVPRACSTEEDDGYLLFYVFDESQLLPSGDCPPSATSELWILDAKNMRDVVAKVRLPQRVPYGLHGTWFSSQDIESQRSVESLRSLEVVQRKKEEWVNSGGQIRKSWMVLREKLEKAVG

SEQ ID NO: 56 - protein, 787 aa, Ustilago maydis
  REGION 1..787: note = misc_feature - Ustilago maydis, strain 521 / FGSC 9021
MVKGSSNRRQHSASLQGLPSSQHCAPVISIPSPPPPAEDHAYPPSSFTIPLSKDEELAEAGPSRPGSSAISRRPVLSRRRTSKKEYVHPYLSGNFAPVTTECPLTDCLFEGTIPEEFAGSQYVRNGGNPLANSERDRDAHWFDADGMLAGVLFRRTPKGTIQPCFLNRFILTDLLLSTPEHSRLPYVPSIATLVNPHTSVFWLLCEIIRTFVLAMLTWLPGLGLGGNQKLKRISVANTSVFWHDGKAMAGCESGPPMRIMLPGLETAGWYTGEEDKEKETCDKNSGNSLTSSSSKGFGGGPPIVSMLREFTTAHPKIDPRTQELLLYHMCFEPPYLRISVIPASQSKKTDLPAHAKTIKGKAVRGLKQPKMMHDFGATATQTVIIDVPLSLDMMNLVRGKPILHYDPSQPTRFGILPRYEPERVRWYESAEACCIYHTANSWDDDGKFDASHEHATRSAIRGVNMLGCRLNSATLVYSAGNLLPPSHVLPPPNCPEKCQLYYWRFDLEHAETNTISHEFALSDIPFEFPTINEDYSMQQACYVYGTSMRDGTFDAGLGKAAKIDALVKLDAQALIRKGKAMWSQGRLKAGDSVDTRTVEEVLTAQRDGSASPEDPIKIFEMPRGWYAQETTFVPRRSSTNETSQEDDGWLVCYVFDEATGLHPSTGEVLPGASSELWIIDAKLMSRVVCRIKLPQRVPYGLHGTLFTEEQIASQKPIDPSQVRSWALSINLADPFSSSALGSTVYSAAGKAATSKFKNREETYAAFIKDPIRIGAWWVKRNIELLIA

SEQ ID NO: 57 - protein, 287 aa, Dokdonia sp.
  REGION 1..287: note = misc_feature - Dokdonia sp. MED134, WP_021779399.1
MKNIRIVITFFLLWLSIQFNSTIEDIIAYVAIFTLGLVHGANDIKLLEKNVSSSPNHKYVILGRYLLIVVAIAILFYVIPSLALILFILVSGFHFGEEHWDKLIVTKGNGHYLFYTAYGLLILFLIFYFNSEETTEIISAITGFTPAKFYYLVGLIFFAILSILSFIVAKAQMNTTSTILQEIVHLIILAIVFKMATLLLSFALYFVVWHAIPSMKSQIIYLYGNTSQKSIVGYLRSSLAYWLLAITSISIFYYYLGDKTYLFNAIFFAFLAAITFPHVWIINSLRK

SEQ ID NO: 58 - protein, 322 aa, unidentified
  REGION 1..322: note = Actinobacterium SCGC AAA278-O22, UPI0003699A87
MEMAKLKTFSRVRTFSSAIVAVAIALSIVFSSWLGVDSLNWQVVMAVVALAIGIPHGALDHLVTLPKAQPLKMAIFIAIYVAIALIAIWAILQWNVWGFIAVVIMSATHFGIGDSAFISELNRLKGIQSHLPIWAYAPAAGALPVVIPLVNSRSTDALQKVNSELINWHHGYTSELQIAVAVIATLSAMTLLSKKRYRDLLDVALLAALASVAPPLVAFAVYFGCWHAMRHTARLSSLLPRSLAAYEAGNSWQAFRSAVIPGLPALIGTLIFVALLAGFSHNNVSDSFLWLTLVTIWALTVPHMMVTAKLDRAALKNKSHLR

SEQ ID NO: 59 - protein, 322 aa, Salinibacter ruber
  REGION 1..322: note = misc_feature - Salinibacter ruber, WP_011404936.1
MHNPVTLRSPRIYGSVLALTLLAVAVGAWGSVSLGPWALGVLFVAVVLTGMPHGAIDHLVAARLWGLDPTWADQAKFYGGYLVLMALYGALWIVAPAWSLGLFLVMTMYHFGQADLAYWRLPPLSARLLYLSRGLFLLGLPIVAFPEVVAPIFEAMGGVQLLEWPGVTTAPGALFAGLVAQHLGALGIGRLGADPSVDWALGREATNVAVLAALFGLVHPLVAFAVFFGGWHALGHILELLRFFRRQGDAMSMAQFYREAFLFTAIPFVGLAGLYAATQSFGLEDQMVALLFILIAIMTLPHMVIVEKMYREREKGAPQAVS

SEQ ID NO: 60 - protein, 705 aa, Zymoseptoria tritici
  REGION 1..705: note = misc_feature - Zymoseptoria tritici ST99CH_3D7, A0A1X7SAA9
MVHAGQKRKRSPSENIPPTPQPRHPYLTGNFAPIQQTLALTPCTYTGQIPEELADGEYVRNGGNPVSNDDLGRDAHWFDGDGMLSGVAFTKDEATGRIQPEFVNQFILTDVYLNTLASPKLRVPILPSIATLVNPLASFIWVTIRIFRTILLVILSHLPGSKQAIKKISVANTNVVYHDGRALATCESGPPMRIQLPGLETVGWYDGANAQGEPKKEDIAKEATLGQDSGLISFMREWTTAHPKVDPTTKEMLMFHSSFAPPYVQYSIVPKSSESEGGEGQTRRSKILNAAVPGVTSAKMMHDFGVSPTHTVIMDLPLSLDPINQLKGLPTVTYTPSKPSRFGVFPRRQPESVQWFETDASCIFHTANTWDDVSSAGETTAVNMLACRLTSATLVFAAGNIAPPIEKLKAPPVKAKRRMPFFSKYDSATEVTIHDLDSPTDEKEPLLTVTTVDEAGEVEEPFEEDQCRLYYYHFDMTTHNITHQWALSSIPFEFPSVRPDVEMQNAQYIYGCSTTTTSFGSALGKATKIDALVKIDAKTLIERGRRTPPPSVTGVVDSRNMTQVLADQHDPTDPVRVFTMPEGWFAQEPRFVPSSSNASEDDGYLLFYAFDEGQLSSLGDVPDDVSPSRAKSELWIVNAKNMKDVVARVQLPQRVPYGLHGSWFTAEQIAEQKSVECIRTTAKALEAREKGVWMSVRSWVEGVLG

SEQ ID NO: 61 - protein, 264 aa, unidentified
  REGION 1..264: note = Planctomycetes bacterium, MBA4684017.1
MNLLWIPLLAILLVGIPHGALDAILIRKVSGSRSSIWFYSSYLLLCVAVVAIWFQAPVVSLAFFLLISLWHFGGADARGYGDDRWLSRLVHGSMIPIVLPALQPEAVAPLFRLVALQNETPFVQWAQILIWPWAALLLLKVIRKEVCKREVLELGGLSLFFWLADPIYSFALYFCGLHSMRHFQQSFQFLSRNRFGKKETIEFAILSGVPILMILLGGAMLDETQWLQGLTGSVFIGLAALTVPHMILIDGWVPLGKPDSGHST

SEQ ID NO: 62 - protein, 267 aa, unidentified
  REGION 1..267: note = Halieaceae bacterium, HBZ50032.1
MLPDLYTLYLPIALMTFIGVPHGALDGYLLHSHSDSKRGTFALLLAYLGIALACVLFWWWQPTLALLSFLLLSLYHFGRSDHQQTGDSVSLTAWIAHGGLWALFLPWQQADATAVLFQALKTDPVILEAFLAPATAIWLLVSGLYLCRQWRNKDQQWRVWVISATAIVLLPPLWSLCLYFCLWHARRHTQWVLRYAKSPSEAKLWMLSIFSVTLAMGAIAYSVLLPSTAIETLTARIFFIGIFALTVPHMILIDYYLGHLHDQQPSL

SEQ ID NO: 63 - protein, 268 aa, unidentified
  REGION 1..268: note = Porticoccaceae bacterium, MAE55128.1
MTTSDIFALALIVFLGIPHGSFDGVIARRLGWSNNLIEQMSFNVIYLSIAILVVIVWFYMPLISLSIFLVISGIHFGMSDISKIQNTGSSYYFPLISHAGLIPVALPVIQKDYVIEVFTILSNNTASKIIFSTLETIYPLYLVIVSAYFFYSIYNKKWLFNAVSLIMLILLAFLIPPLLTFALYFCFWHSRGHILRVWQRVKINERRSHIIETVGYTIFTWLILASGLWLLDGSLNESIIKMVFIGLAALTVPHMILVDFWANRRKVL

SEQ ID NO: 64 - protein, 269 aa, unidentified
  REGION 1..269: note = Gammaproteobacteria bacterium, RCL40986.1
MLELGLLNTIALISIIVLGIPHGGLDAAISRKKGWSKTKINTIYFHVFYLSLTSLIVALWYFFPLISLILFLTISALHFSYSDLNNSKDIRIDALISHGGIVPIIIPYFQADLVLEFFSILSGQENAAIVITIINYLIFPWAAFFVIYTFNVIRKKKYSFNYTQLLLMLILALILPPLVSFSIYFCIIHSPRHIFIILNKMNSHERLRAIKETTFYSLTAFLLMFIVGYFLASSYIITDSIIKVVFIGLAALTVPHMILIDYFESLEKE

SEQ ID NO: 65 - protein, 276 aa, Euryarchaeota archaeon
  REGION 1..276: note = misc_feature - Euryarchaeota archaeon, MBJ64590.1
MSLTTALSSMDTLNLIALMCVVFIGLPHGAMDGALAAHFGWMESKKKAATFLIGYVAMAALVVGFWFVAPAISFIIFLGISMYHFGKGDVNTDGEEPSVTESLARGGVVIGGISQFHRVDADEIFQVLIGSNTELVWLFLNSVVPMTLIFAILSMVNKTTGERGSLLAEIAGLALIFSILPPLVGFAIYFCLIHSIRHFASMRAMLSDTITKLQITKTTILFSAISWAAGLIILAQQSSNIGFEPALLQVIFIGLAALTVPHMILVDGYTEYQKSQ

SEQ ID NO: 66 - protein, 286 aa, unidentified
  REGION 1..286: note = Alphaproteobacteria bacterium, GIR97087.1
MMDILSVTDLFALAAVMLIGLPHGAFDGALAFCLGFGRSPGKIIGFLVMYLLLAGLSALIWLVSPAFALAAFLVLTIVHFGSGDTEHLYQLGPRLVQRSLKACQILVHGGMVTILLPVFHTEEVSQLFIVLAGPNAVLIMDALRPALTIWLAAACIYAGAALFNRQYGAAAGELAGLAVLVWLLPPLAGFAVYFCVVHSRRHFTSIWKAMQLFVSRRFILISGGILTAASWAMGAGLYFSQTMSGSFSPDEAFIRAVFILLAALTVPHMLLVDTMYRPTLKKITQP

SEQ ID NO: 67 - protein, 286 aa, unidentified
  REGION 1..286: note = Uncultured marine bacterium HF10_19P19, ABL60983.1
MIDILSVTDLFALAAVMLIGLPHGAFDGALAFCLGFGRSPGKIIGFLVMYLLLAGLSALIWLVSPVFALAGFLVLTIVHFGSGDTEHLFQPGPRVVQRSLKACQILVHGGMVTILLPVFHTTEVSQLFIVLAGPNAVLIIDALRPALKIWLAAACIYASAALFKRQYAAAAGELAGLAVLVWLLPPLAGFAVYFCIVHSRRHFTSIWKAMQLFVSRRFILISGGILTAASWAMGAGLYFSQTMSGSFSPDEAFIRTVFILLAALTVPHMLLVDTMYRPTLKKITQP

SEQ ID NO: 68 - protein, 293 aa, Henriciella sp.
  REGION 1..293: note = misc_feature - Henriciella sp., NQY41612.1
MTWNSAPLNFLLAGMTVSIAFLLVPSLSITLQLVVLGVAVAFLGLPHGAIDAYIARRDGLWRSTRGFAAFVGIYAVVALSVIGVWMFMPTLSLLAFLIISAWHFGADANARDQAERWLFGGLLLSLPSFFHPGDVASLFEAISGASAGGLVPFLQIWAPVAAIGVFAMLVRRRPPAQQRWTDIATVAGLILFAWALPPLVYFVIYFCALHSPAHFGRVIRLVTPSDRTRAAAYTAGFTALTLLVAGLAFIALTDKLTIQQSTLQIVFIGLAALTVPHMFLVDGICRAWFGEAE

SEQ ID NO: 69 - protein, 294 aa, unidentified
  REGION 1..294: note = Cryomorphaceae bacterium BACL22 MAG-120619-bin32, KRP28444.1
MVVNNIKNYQNFKIFITVFLLWFSIQFGKPIEDSIAFVLIISIGIIHGANDLLILSLKKMKNNRFVINLFIYLCIVSFCVIFYILNPFLAILLFIFISSYHFGEEHLSEKMITNRYFESFYYIVYGLLIFSMLFYEALFEVNTIMLELTKTIFSEFFVKYTFIFSLISFLLMNTFLLWKEKIDAKVFLKEGFSLGLLFLVFKTSSLILSFAIYFIFWHSIPSIINQILFISGDISKSSILFYVKKAFLFWLISIVALFVLYQFIPEVTLLSTIVFVILFSVTAPHIWVMYKMKN

SEQ ID NO: 70 - protein, 294 aa, Loktanella sp.
  REGION 1..294: note = misc_feature - Loktanella sp. SALINAS62, WP_211643588.1
MTLSPIWQTMVFAATALAMIGVDLLFQPDLMLQVLILVPAVAILGLPHGALDLPIAQSLWSLKGWRGTAAFSAIYLGLSLLLVAFWIAFPGPALCVFLVYSAVHFSGDWDDAGRAQRWIGGVATVGAPALFRADEVAAIFSYLAPADAANFAAQGLAIMGAFALMSYLSLFIFRSNFRDRAAIEIGVIWIAAACLAPLVYFIVYFCALHSVRHFTDALASLDRKKQALGVAIVVSGVTVLIGLLAFINLPDAGSDTLQQPIVQVVFIGLAALTVPHMILVECFQSRAVRQPDRC

SEQ ID NO: 71 - protein, 295 aa, unidentified
  REGION 1..295: note = Flavobacteriaceae bacterium, MAH20764.1
MKIIYSFSIVSSFMGLWITSLLPEAFELFLGFILIFTFGMVHGSNDIMIVDKLSKKTNSTFFKTLSTYLMVVTAAILIFYFIPLLALVLFILFSAYHFGEQHWEEVLSNLILPLRKIFFFCYGLLILYLVFLFNVEAVIEIIYEITSLKLNDIYASEVTQILFVILFLITGYAVYKKQIAIKSVLRELFSLLIFGIIFNSSSLIWGFTIYFIFWHSIPSLLDQITFIYGDLKKKSILKYVKAALPYWLVSLVGIAVLFLIFKHEKHFHSLFFAFIAAVTFPHVIVMLRMFSKKRI

SEQ ID NO: 72 - protein, 295 aa, Robertkochia sp.
  REGION 1..295: note = misc_feature - Robertkochia sp. 3YJGBD-33, WP_224483742.1
MNSYRFESYLLVLTFFSLWLLGYLNAVTARSTAILLVLTIGLVHGANDMKIISRLSENKQRTRKYVAGYISVFFLTSVAFYFFPLVALILFIFFSCYHFGEQHWLEHIDKPEKGTKLFAFFYGMLIIMLILQFNAGESVMIINDLLQLELKNLPFAIFTLIALFPVLVLGYRYRKLLFPSYEKLAKEILLMLVFIIIFNLSGLVWSFAVYFILWHSLPSLRSQLTYLYGSTTMAGVKRYIRDSLPFWLLAIIGLGVMIWFLKDQREIIISIFFPFIAALTMPHVVIMSMMFGEEN

SEQ ID NO: 73 - protein, 297 aa, unidentified
  REGION 1..297: note = Flavobacteriaceae bacterium, NBT87107.1
MNKNFNIIITAFVIWISLYTNAQIELLASGIFVLSLGLMHGSNDINLLQELLTKNKTKKWVLIGFYVSLGAIVFTLAFYWRVLGLIVFMLFSSYHFGEQHLHNRLSDSKIKVFDFTLYGLVIFSMMFYTHQNEVQFLIFQMTDWDITGFPMSYVTIGLIGLLILSWIKHYKFFKKEIFEEMFYLVMFYIVFFNTELPLSFAVYFVIWHAIPSIKDQILVEGYTLSYKSALRYFKQSYLYWLVSVFGIFLLYKVYDLIGNGLFPILFAALVAITFPHILLITQLFQKLNQSQKTKEGH

SEQ ID NO: 74 - protein, 298 aa, Eudoraea sp.
  REGION 1..298: note = misc_feature - Eudoraea sp., MBT8293718.1
MQNKGVDFTKLQGATLIATFFSLWLAVYFEHAVENLLAYILILSFGMLHGANDIKLLQVEAKKKSKPYDFFRILAYYVLFVLSVAGLFYVLPSLALALFILFSAYHFGEQHWISKINGNKLFLIFFYTAYGLVILFLLFAAHHTEVSDIIFDIIGFYIDNAYYRITLGISAFIFVALYLIKYKQSKINIFLELFYLLVFFIIFNTASLLWAFAIFFILWHAIPSLADQITYLYGDLSRKHFMKYVRSSLIYWLASVITLVILFFFIDGSSEGFIPFLFSFLAAITFPHVLVISGLNKS

SEQ ID NO: 75 - protein, 298 aa, Psychroflexus salaries
  REGION 1..298: note = misc_feature - Psychroflexus salaries, SHE30376.1
MFKIMSKLYNYIIVLNFFLVWIGFYLPEQIKTSIGLLGILSIGLIHGSNDIFLIKNIQRQQSILKYIFYYSLFIISFAVAFYLIPIVSLPVFILASAYHFGEQHWQKAFLKAPNYIKQLFFICYGNLVIGMILYFNQVESTTIIENLSGFVVNESIYLFNILVSLIITSISFILCAKKINSKASTIVLNIFYLLLFTATFYVSDLIWSFALYFVLWHSLPSLKDQIDFSFGSINFQNLLMYLKQSFVYWLISIIGLIVFYFLLKDTKYILSILFAFIASVTIPHVFVVKKMFEKKAST

SEQ ID NO: 76 - protein, 298 aa, unidentified
  REGION 1..298: note = Verrucomicrobia bacterium, NCX47571.1
MPVLLRQTAPWFLAALITLLSLLFPQPACTLAPWFFLSSALILGVPHGACDPWIPGWILHRPSRLPFLIPFFLIYLAISAAYLLLWRSYPLPSTFFFLLLTAWHWGTADASLETTPSLRWLLFGIGRGSFVMLAPFAFHLADTWSVILLMAPNAGPPPSTFPFFPALLLALALNLFSRPSIHQTIETLLLLLLFYLTPPLLSVGTYFVAFHAWRHLLRLSALRNQLTSNEPLFRWTHSLSQLLLLSIPLTLASLAFLRWIPPFLTSSFPKGTNKLGRPLSYSPRRPYPTPRNPCWMDR

SEQ ID NO: 77 - protein, 300 aa, unidentified
  REGION 1..300: note = Flavobacteriaceae bacterium, UCE68985.1
MNKKGLKIPDLRNAMLVSTFFFLWLAVYFSQSVEEVLAFVLIFSFGILHGANDLEILRRGQAVTETGNPNVKLLLSYIGFVLGSALLFSLLPLVALTFFIVFSGYHFGEQHWVRRMGRRIWLQKFLYTSYGLTILMLLFAAHAEQVAEVIFDITGFSVQPAWFTYGCLGFLGLFLIICFFNFFWTQFKRYVFRELFLLLVFFIVFNTASLIWAFAIYFVVWHSVPSLADQIRLLYGSVNLKNGSKYMRSSAIYWVGALVTLALAFLYFRESSLGFMPLFFSFLAAITFPHVLVISRLYRS

SEQ ID NO: 78 - protein, 301 aa, unidentified
  REGION 1..301: note = Sphingomonadales bacterium, MBM3922853.1
MLPILKQQSWLTLTGLIMGLIFWLSPIPQSWQWIFFLLVIISVGLPHGALDFLVAGKGRNNAKLGRFLLFYLSQSVAFLLLWFWPLAAVSVFIVLSAYHFGETDLQLFNPAPLRKHIAIICYGMATLLALLFSHFTELTALIPEIAQFVQQFKILQWLQEYYLPIYAGLMGGALLLLASEKNIDRIKLASLLLLCIPQLLIFYMPFLAGFTYYFGIWHSLLSIVTIQNNTNTTGWANIRKLINPQTILFALLALALMGLLYWGISSFFNYDNQLLIFFIGLSILAIPHMQLMHELLSRRFS

SEQ ID NO: 79 - protein, 304 aa, Ferruginibacter sp.
  REGION 1..304: note = misc_feature - Ferruginibacter sp., MBC7922457.1
MRYVHSTLFHVQWLLQVLLTGLFCLSDWGSTTQAVLCGALLCTVGIPHGANDHAYRPNTTSVGLLGFLGVYLGSMGAYLALWWSAPALALLLFFAISLHHFGQSNFENRSVWYLPSLLWGAWVLVFPVLLHWDEAMGIFGAMVGSGVSWEKSASGLSGMWRGLAALLLALFYLLALLRLEREQWTRYLLQWGVVSVWYWLTPLLFGFIVVFCVWHSLQSLQHQLRHFQSAGGGSRRQFFLGLLPFGLLALAGFGGYVYFRGFAVDEAFVLLSLITLPHVVVMHRLYGAPASPTSPTTASGKRAV

SEQ ID NO: 80 - protein, 317 aa, unidentified
  REGION 1..317: note = Actinobacteria bacterium, MTB30923.1
MESAKLKVFSRVRTFSSLMVALAIALSILFSSWLGAESLNWQVVMAVIALAIGIPHGALDHLVTLPKSNPVKMSIFVFIYVAIALVAIWAILQWNVWGFIAVVIMSSAHFGVGDSAFISEYDDLRGSSSPLPVWAYAPAAGLLPVVIPLVNSRSTEALSKVNIALVDWHHGFTSEIQIIVAIITTLSVITLISRKRFRDLLDVSLLALLASVAPPLVAFAVYFGCWHAMRHTARLTSLLPRSQSSYEKAHARKAFISAVVPGLPALAGTLIFVALLAGFSHQNVSDNFLWLTLVTIWALTVPHMIVTAKLDRAALKN

SEQ ID NO: 81 - protein, 319 aa, Spirosoma fluviale
  REGION 1..319: note = misc_feature - Spirosoma fluviale, WP_097126042.1
MITSLLTKLSSYIQRTPTGLTILAGTLLACYQYWVGPLPIWLQLVFCISLLLLVGIPHGALDHLIEQERTIRQAQPFSLARFLTKYVLMIAIYGLAWVFFPVFSLTIFLLISAWHFGETDLENAPDDRYWSLIRLVAGGFVVAFILLTHAPEVTPILGRIIQNNPQALNLWGEAAAGAGSLLRGWATLAVVLAMLALGHQPMRLNGWRLARLGMVLLLTYMLPLLPAFMLYFGGWHSLSSFGIIQSYLQRPGKPTQTIWKLWRQSIPLTLLAFGFLMAGAGIWQSYIPLFDPLPYLFILLSTITLPHIQVMHQLDLLRK

SEQ ID NO: 82 - protein, 319 aa, unidentified
  REGION 1..319: note = Actinobacteria bacterium, MBC7464309.1
MEDALPKIFSWVEWWSRAAILTTVILIIFLRIIHIELSLSVQIIMALVGLIIGIPHGAIDHLVSIPSQPRVRFYTYIVAYIAIALMAGWAISTWNMFGFRVVVIMSALHFGFGDAAYRNEWKSATNSRKYPWFIETAYALPAGFMPVVLPLTDPRSLSALNRIHPGLKDWAGANVHLFRGITLVMFICALVVLLIAKSFSFALDLGLLGLLTLIAPPLVSFAIYFGFWHAARHTVRLIPKLEKARALALNGDGLKALRGAITPGLYAVAGTALLASGLTLVTKEISSSGFLWSTLVIIWGLTVPHMLTTSSFDFQAIRK

SEQ ID NO: 83 - protein, 321 aa, Marinobacter psychrophilus
  REGION 1..321: note = misc_feature - Marinobacter psychrophilus, WP_048386795.1
MVINDTANTENVLYRSSGGRRHQFIILMIASLALIVGFMLPVLSIEQQFWIVLLPIGVFGLSHGGADPMILKQLTATSPTGLFVVLCAYLMASLAFVALIWVLPVLALLVFLGLSIWHFGYTDEAFLSSAKNPPLRWLFGSTPILGPMLGHPQQTSELFAWLIKNESQVVLDIVVWLGPCLAVVWLFGFGCLLFGRLNRPGPRVLVELCLVGAALIALPPLLGFAFYFCAIHSVRHFLSIAEHRLLSNQKSLFQAFPVRKILPATLGAIAMACVAWGVIVLIGPATSLMADAVRVMFWALAALTLPHAIIVRLWWNQRLVE

SEQ ID NO: 84 - protein, 324 aa, unidentified
  REGION 1..324: note = Actinomycetales bacterium, MBU6347572.1
MSESQLRLLNRTRELSFFLILSGLVVAFFASRMFGDNADWQIALALAALTIGIPHGAVDHLLSVPKFFSLRMAIFLFGYLLVAGMAAWFILLWPLLGFQLIVLISALHFGVGDASFYMELTGRLRKPGFPRIVYALAAGCTPVLIPLTNSQTKAALQNVNPKLIDWAGPFAKQIFILCIALNLATAALLAIKKFYWPTLDLLLLLALSLITPPLVAFALYFGFWHAFRHTTRLTLEYRPALQLHVPDQPWRSFWKVVRAGLPAVMVVLIFTAWLKFTNGTSTSEDFLWYLLVVTWALTVPHMALTAKSDAVALKLKPSRIREST

SEQ ID NO: 85 - protein, 343 aa, Geodermatophilus chilensis
  REGION 1..343: note = misc_feature - Geodermatophilus chilensis, WP_100501915.1
MASPALGRRGASAPAPEGLPPGQALARPVRAATGVSLGAAGAACLVELTLPGGWGDAAPVVLVAGLLLGLPHGAVDHLVPGFRSGRRPAVLAGFAAGYAVLAAVGWLAFRAAPGAALLAFVAVSVWHFGSGETAFADLRAGHPVRRRLAAALVVGALVLLVPLVRGAEETAAVVAAVVPGSDGRLPPELTAVVLGAVLPATAALAASLLAARRWLDAAELGVLTALVLVVPPLAAFGVWFGCWHAVRHVARVVAEDPGARADLVAGRLARPLRRFAVQAALPTAAVLVVLALLWEAAGGWRAFVATDVPVLAALTLPHVLVVGWLDRLESTGTGPGGRTSGGP

SEQ ID NO: 86 - protein, 350 aa, Geodermatophilus sp.
  REGION 1..350: note = misc_feature - Geodermatophilus sp. TF02-6, RBY79850.1
MARRCYRGRRRRQRGATGLTVPSATVRGRAAVTAPSPGRRSATPFAAPARTATVVSTTAAVAVLLAEVAVPGGWGDAAWGVLVGGLLLGLPHGAVDHLVPGRRLGWRPVRLAVFAAGYAALATVAWLVFRAWPGPALVAFVAVSAWHFGTGETAFADLRAGRPVGRRPIAAAVVGAVVLLVPLVRGSADTAAVVAAVVPGSAGRLPAWLPATVLGVVLPAAAVLAARLVGGRRWVEAAELVLLACLGLVVPPLAAFGVYFGCWHSVRHVARVVAEDPAGAGDLAAGRLGRPLRRFAVQAALPTAAVLAVLALLWSAADGWPSFVATDLPVLAAVTVPHALVVAWLDRAPS

SEQ ID NO: 87 - protein, 350 aa, Nocardioides sp.
  REGION 1..350: note = misc_feature - Nocardioides sp. TRM66260-LWL, WP_224127607.1
MLRPVPAPVPASSPLRALRAGVIGSRRAALVGVGVGALLTGAAAVGLASGPAEALTWSLVGLGLLVGLPHGSIDHVLAARMTGWSLPVAAAAYGAVAAATWGLLHAPHGVGAATLVGVLALSLLHFGLGELEVFRADAAWHPAPAVAAALALAGTGALLVPLARAGDDLVGVATSISPTLGAVLSSPAARLGLIAVWGAALLVAVAAAWRAGRSDVARDLLLVAALGTLAPPLLAFAWWFGGWHGVRHLARLLGAEPGSARLLEREGVGPAARHLARIAAAPSLAAAATVVALLVLTARAGDPTAALGGTLVTLLALTVPHMAVVLWMDLRTWRTERTRRPAAGRALRPR

SEQ ID NO: 88 - protein, 561 aa, Aspergillus pseudotamarii
  REGION 1..561: note = misc_feature - Aspergillus pseudotamarii, XP_031915663.1
MTGKEKSHKHPYFSGNYAPIYTVQHAHPCEVQGTVPEEFLGGQYVRNGSNSLQDDDRRDLHWFDGDGMLSGVFFKRVPGSKVQQPLYSNRYILTDVHCATAAYPHINPILSSATTLLSPMVSPLKVFMGMLRTMALMLSSFLGFVDRPIRRISTANTNILYHDGRVLATMETGPPMRVYLPSLNTVGWFTGGSAEGEATDQIMEPSIGDPGIERFHNEMTTAHPHCDYQTGELLLFHSTFIFPFVHYSIISPGWNGKHGSYLNQPVPGFTSGKMIHDFGVSRKHTIMLDIPLSMDPTNITRNKPAIQYDPHGRTRFGVFPRYCPAQIQWYETDPCCIFHTVNSWDDSVPWGTRVHMLFPADRSGAITEQWALSAIPFEFPHVPQHLEMTAAQFVYGCSMSEGNFATRQKSSVKVDCLVKIDVQRLLQAAEVQPPTQITGCVDQRSINEILATNDVNDPIQVFALPYGWYAQECSFVPRKDGTSEEDGWLVTYVFDESQLDSNGNAPATSRSELWIIDARNMRDVVARVLLPQRVPYGMHGDWFSEEQILNQREVAEFRSLD

SEQ ID NO: 89 - protein, 606 aa, Ilyonectria sp.
  REGION 1..606: note = misc_feature - Ilyonectria sp. MPI-CAGE-AT-0026, KAH6971494.1
MTSTKSPKTHPYRSGNFAPVRNQMSMEICEVQGTIPDELIGGQYVHNSSNPLAYDDDPSRPMHWFDGHGMLSAIYFSRQDDNNTLDIQAMYSNQYVLTDIYLAESNSRVRAPILPSLVALLSPWSSVFTILRGYFRMIWMVLCSWVGHSLIPIRRISVANVNVVYHDGRFLALCETGPPMRVTLPDLKTVGWFNGCMAEGEFPEDSKTVKRSGFAGDGLLSFFREWTTAHPHIDPLTGEMMLLHPTFIRPYTRYSVLAGSSSNAGNGRQIFGKSVPEVLSPKIMHDFGVSRRNTVIIDMPLSLDPFGLAFGESAIAYNSKGQTRFGIFPRHVPELVQWFATEPCCIFHTVNTWEDTVKGETAVSMLVCRMTGPSMVYATGNLPLPTSQGHREEECRLYYYQFSVGGPHRAITEQWALSAIPFEVPHVPKHLTMSATRFVYGCSVAGGNYADHLENALKIGSFVKFDVQKLIARGLANHPTSVSGCVDNRSVEDILASDDPDDPIQIFPMPKGIYGQECVFVPRSGGKSEDDGWLLTLVFDESQLDVDGNAPDDCKSELWVIDAKTMKGIVVRIKLPQRVPYGFHGNWFSEHEIAQQHPVKKFRSQK

SEQ ID NO: 90 - protein, 609 aa, unidentified
  REGION 1..609: note = Ophiocordyceps camponoti-rufipedis, PHH79717.1
MAHHPYRTGNYAPCRRESHLAGCFVRLGSVPDDLAGGQYVRNGSNPLGYEGDATGRDMHWFDGHGMLSAVYFSVPEKPGATIMPLFSNRFVRTDVFLAEAAAASPLRVPLLPSIATMLQPSLRMYLRLARCILSLLLTVLLSLIPGSRRQSIRRISVANTHIVRHGGRFLATCESGPPIRIRLPELATVGWFNGLGAEGEPRRGVWPGGHGWRAGLLGFLREWTTAHPHVDPQTGEMVLFHSTFVRPYVRYSVLAGAPHPDHGPTPSRRLPFNSPVPGVRSARMMHDLGVSRRHTVIIDMPLSLDPFQLLRGRPVVAFDRTSPTRFGVFPRHEPAKVRWLVSAASCCIFHTVNTWDDPAAVHMLVCRMGEPSIVYATGGLPTPTTDMDDATLTARNHCRLFYYQFRLDQDPAVAQQWALSAIPFEFPHVPGHVAMSAARFVYGCSVRGGTYAEQLANTMKIGSLVKIDVAVLIARGLKQPPAPTWGCVDERTVDEILADDDDDDAIRIFVMPEGHYAQECVFVPRKGGCREDDGWLLTFVFDEAQLDDFGNAPDDARSELWIIDARSMRDVVARVVLPQRVPYGLHGSWFPREELENQRPVATFRESKA

SEQ ID NO: 91 - protein, 611 aa, Penicillium sp.
  REGION 1..611: note = misc_feature - Penicillium sp. RFL-2021a, KAF7719097.1
MTPWFLSGNFEPIQRQQELTSCPYSGTIPEELAGGQYVRNGGNPQQMPKETERSHWWDGDGMLAGVYFNQSDDHDSKLQPQFVNQYLLTDVYLAAKSMSSVFSPVVLSLSVMLDPCISIFDFLCELIRCAVLVLWSHISPMVPAIRNLSVANTSLSFHDGRALAHCQSGPPLRVLLPTLQTVGWFNGASAEGEPETHSSFEEVIGGRGPLAFLKNWTTSHPKVDPDSQQMVTFQSSFLPPFVWYSVLPDSGHDSGKSSLSQTKLINQPILGVTSPKLMHDFGVSGSHTIILDLPLSLNPLNTIKGKPPVQYNPQSPSRFGIFPRLQPQNIRWYETDACLIFHTANTWDGVEISPYSKESATVVNMLACRTTTPAVLYTSSSSSMSLGAEFFEQGHDACRLYYYSFNISDPINNQIMHEWALSAIPFEFPTVSPKYEMRNARFVYGCSTTDSKSSASIGQGVKVDCLVKMDVQKLIQKGRKESPTPIVGCVDTRSIDEVIASKIMDDPIQVFQAPKGWYTQEASFVPRQYATREDDGFLLAFMFDESQLHHDRSCPLNARSELWVIDALDMETVVTRILLPQRVPYGFHGHWFSREDIERQRAVASLRRGTM

SEQ ID NO: 92 - protein, 615 aa, Ramaria rubella
  REGION 1..615: note = misc_feature - Ramaria rubella, KAF8591152.1
MKTHPYLAGNFSPIHTEKDLTPCQFVGSIPKELWGGQYVRNGGNPAHSTALGRDYHWFDGDGMLTGVLFKRIRSQVEPCFVNKFLLTDVYLSGRRSGALFLSSPLLPSISTLVSPTVSLHVLLKAIFRALFLFIKNSGIRLSVANTNILFHDQRALATCESGPPLEVTLPELDTVDWWTFGNRISGHTKERRGLGGERGLKAFFEEWTTGHPRVDPITSELILFGSSFLPPYVRYTVISSSGTALPSLLGAPVPIRSPKMMHDFGVSLQHTVILDLPLTLDPLNIARGKPIVEFYPEGRSRFGVFPRYHPGLVKWFESSACAIFHTANTWSDETAVNLLACRFASAKLVYIAGDISLPPSCIDSEDVCQLYYYRFPLSPHDATPSISHEFALSAIPYEFPSISHSVSMQDAQYVYGCTMRSGSFDAALGAAKIDCLVRVDVRTLIRRGIDSGICHGGVVDSRQVVEILASTDEVDPIKIFEMPQGWYAQECSFVSKQEAKSEADGYLLAYVFDESQLNDDGSAPNCARSELWILNAETMNEVIGRVKLPQRVPYGLHGNFFSEVQIASQRPSRPRRTISLHNNGVNTFLPTFLCLAMVCNIYVFSAMLGEASNLT

SEQ ID NO: 93 - protein, 619 aa, Aspergillus alliaceus
  REGION 1..619: note = misc_feature - Aspergillus alliaceus, KAE8385184.1
MNISGTGVEGNSRKHRYMSGNFAPVSETHSSEPCYCIGSVPEELSGGQYVRNGGNPAIEVEIAGKTHWYDGDGMLSGVYFRSSPGGAHIVAEYSNQYVMTDVLLATMDHPTLDEPIPLSIATLLDPHVTLGQFLKRLWRMSKVIATSYLEFYSHTIKRISVANTNIVYHDGRALALCESGPPLRVSLPDLRTVGWFNGLISEGEPMEDTDTSGFGASGFFRLFQEMTTAHPRCDPQTQEMIIFHSVFIPPYVSYSVIPKRGKTSSRPANMLLNVPVPGLSSPKVMHDFGVSRKYTVMIDLPLTVKWSNLWHTKPVVSFDVSSKAAFGVFPRHNPDQIRWFHTEACCIFHTVNTWDTEPTGDEKGLQPRVNMLVCRMNSPAILYTTADLDPPVVPAQDGIPQCRLYFYQFDLTRPWNIISHQWALSAIPFEFPHVPKQSTMSETRYVYGCSTTEGCFGTALGKAVKIDCIVKVDAKGLIQQGLETSPEPIFGCVDCRSVQEIVQDRKSNDPISIFRLPTGWYAQECSFVPRQGTVDEDDGWLLSFVFDESQLDSAGEAMSNARSELWIIDARDMQTVAARVVLPQRVPYGMHGNWFSREEIEDQQPVESYRCLPKKVGHS

SEQ ID NO: 94 - protein, 627 aa, Peniophora sp.
  REGION 1..627: note = misc_feature - Peniophora sp. CBMAI 1063, VDB91879.1
MNHAYLSGNRAPVTNEVPLTPCRILQGTIPPQLSGGIYVRNGSNPAPNVNTDNLRPYHMFDGDGMLAGVYFDFERGPLFTSRWLQTDVLAAAKRFSLSRATFPSITSLIDARAPQLLVLLEYLRCILVVALSWILALWNKAGGGIARISVANTAIIWWDRRALATCESGPPMRVGLPQLDTRGWWLLGGALPRVSMLQSIFKLKAFFQEWMTAHPHVDPETNEFVAFHASFFAPYLYYTVLQPSNSSSRSQLVRKPVPGLRAPRMMHDFGVTPTHTLFLDFPLSLDILPSIKRQSISPSLTYDPTIPSRIGVLPRYAPEEVVWFELDRPGGCVFHTTNAWDAPEQRAVEMLVSRMGGPALVYAAGALPVPTHAGTDECLLYYYRLPLHDTLAAQRHPRPSHAFPLLSLPFEFSAIHPARSMHSQRWVWGCSFFDHPVPGPWDPYTSTSHHGPETARLPPAELTGYSDVLSSGFKVDTLLKIDTSRLITQGLERGEGEHAVPVDNRSAQELWDAQEAGDDTSVRMLRLPEGCFAQEVTFVPRHEVLPEELQDEEDDGWLLTYVFDERQLNEAEKGGSCGLESAISELWIIDARTMDIGTVCRIALPQRVPYGLHGEWFSAGRIAAQEP

SEQ ID NO: 95 - protein, 636 aa, Rhodotorula sp.
  REGION 1..636: note = misc_feature - Rhodotorula sp. CCFEE 5036, TKA55938.1
MGGATNPHPYKTGNFTPIREERILERCEWTGDIPEELVGGMYVRNGGEPALAELQNGSGDGPAYHWFDGDGMLSGVFFEKASSSTSDPKQRVQPTFINRYVLTDVFLASKELGVRDPILPSIATLLGGVTSLHLVLFAIFRAVFLAFCSFFTKSPLRHLSVANTSLLWHDGRALATCESGPMTWVTLPELDTVGYYSLEGENGETGLREGMLGWMKEWTTAHPKYDPHTGELMLFHMTFLPPYLSYSVVPSTHTPTPSEKKVTPRLLAAPVPISAPRMMHDMAASRTHSILLDTPLSLDPRNLALGKPVIGYEPASPTRFGVFPRHAPNLLRWYLAPACIIFHTAFAYDEYTANSPESLAAVNLVCCRLNSPRLVYSAGNLMLPKSQELPAGAKEACELYYYRFPTSNSASQNLEPSHQFPLASIPFEFPTVPQDRVVGPAKYVYGCSVKHGNFDAALGGAAKIDCLVKINATALIQRGIKRAETGKQDAETPVDQRTIAEVLSSQTPRGGSKSSSSATEIADSDDVPIRLFELPPLHYAQEPSFVPRSQPRAEDDGFLLTYVFDERQVDLVSGRPFKDARSELWVIDAWDMRTVVARVKLPQRVPYGLHGHWFAADDLQHQRSPPSIRTRPTATS

SEQ ID NO: 96 - protein, 639 aa, Acaromyces ingoldii
  REGION 1..639: note = misc_feature - Acaromyces ingoldii, XP_025380094.1
MRHPYTSGNYTPVKRELPLTEGVVVQGSVPTCFHGGQYVRNGGNPLADGDEKRDAHWFDGDGMLTGVLFSEPTPGEKEARSRDARPMFINKYILTDVLLSTSTKYRQPILPSITSFALPFQWSALPYLLAMLLRSIVLVFLSWLPFQRRPRVGRISVANTSVYWHDGRAFAGCESGPPIQILLPGLETADWWHGGWAKGQGPFKMLNEFTTAHPRVDPVTNELLLYHMSFAAPFLRVSVIPPRSSRKPALLGAPVRGVARPKLAHDFGATATKTILIDLPLVLDPRNLVAGQSMLSYHRDMPTRFGVMPRWAPQEIQWYESESCSIFHAANAWDDDEDAACLLACRLNSATLVYSAGNIATPDNCKPPLGQEERCHLYFWRFAPRSNTISHEFALCNIPLEFPTINERFSQRKNRFVYGASMTCGSFDAGLGSRNAKIDCLAKVDVEALVCRGRSRQDAGLLSKGECVDDRTVSEILQSGEKEDAIKIFALPPLCYGQEATFVPRVEGGDEDDGYLVFFVFDESKGLNADGDCTPDAKSELYIIDAKDMKTVVCRIELPQRVPYGLHGHFFSREDIASQIPIDASKVRSWALARKGLGPGPGLGNGGKPALAKAPSNFLVGALEAWSDALRGTIEGILA

SEQ ID NO: 97 - protein, 646 aa, Acremonium chrysogenum
  REGION 1..646: note = misc_feature - Acremonium chrysogenum ATCC 11550, KFH47490.1
MDHQHPYLSGNFAPVQSSLPLTACPAEGKIPSDLAGGQYIRNGANPVTPNEHLDRDTHWLDGDGMLSGVYFRRAGDKGTEIEPQFVNRYLQTDVYRYARHSRFLTRPVLPSLTTTLLLGGGPVVRLVRVLRAVLRTLILAVVSRLPGRPSIRKMSVANTSVVYHDGRALATGGAGPPLRFLLPGLETVGWFNGRRAQNEPRGDGRSGFRGDGPLAVLRGWTTTHPRVDPVTRELIVLRSTACKPYVRYSIVKPSKQTEPGSSIFDEPVPGVTTPKMVHDFGVSSHHTVIMDLPLELNPKNLLKGTAAVRYDLTSRSRFGVFPRYAPYGIRWFETSPCVIFRTANCWDTISSATKERPAETLVNMLVCRRTSATAVYNTGNITAPWRGPVPPEYAEEEQCRLYYYSFPITDASHTERHIRHQWALSAIPFDFPIIPADKSMSECRYVYGCSSSHSSTPAPGSAAKMDYLVKMDAKSLVACGIADPPPAVTGCVDTRTLEQVWAESSNGDDDDDDDPIKLFRMPRGWYAQEPMFVARADTESEDDGWLLTYVFDESTGLDEEWECREGAKGELWIIDARNMRDVAARVKLPQRVPYGLHGSWFSEGEILAQRPHGSVRDAGAVVSKRTVWKWEGILGGLGSVVERLVG

SEQ ID NO: 98 - protein, 662 aa, Sporormia fimetaria
  REGION 1..662: note = misc_feature - Sporormia fimetaria CBS 119925, KAF2741777.1
MTPKKPSHPYLAGNFAPIHSTHASTQCSFTGTIPKELLGGQYVRNGGNPITDQDLARDAHWFDGDGMLTGVHFVRSPSNPGEALPFFTNQYILTDLCIFAHQNRNLRTPLLPSIATLVDPASSLFTILFRLLRAVFLIFVSHLQRPGKIRVKRNSVANTGIHFHDGRALASCESGPPMRISLPGLETVGWFDGANCEGELQSNGEKKEDTFRGTGLLGFMREWTTGHPKVDPVTGEMVLFHSSFKPPYVHYSVLPQNRAATAERLPKLLVVPVPGVSGSKLMHDFGVAREHTVILDLPLTLSAFNMMKRQPVLYYDCDKPARFGVFPRRQPEKVRWFETAGCCIFHIANTWDERDANGNIQAVKMLACRQTSASVVFNTGNITIPEMPRSRGRRLQNLARRGSDEEEGTPDDKTPLLDSSTRSSAGDTEQCRLYYYRFSLDRPGDVITHQYALTRVPFEFPTLNPAYDMSAARYIYGCSTTEDTFGTALGKATKIDVLLKVDVTTLMERGAAYPPRSVTGCVDMRSMAELFASKDQQDPIRTFLLAPHHFAQEARFVSRENPVAEDDGYLLFYVFDERQLDGKGECLPNAMSELWILDARNMKDVVVRVRLPQRVPYGLHGNWFSEEQIQTQRDIEGFRSLPVDPEKGVMARLRSLLVGAVG

SEQ ID NO: 99 - protein, 665 aa, Tilletiopsis washingtonensis
  REGION 1..665: note = misc_feature - Tilletiopsis washingtonensis, XP_025600717.1
MASSSAHPYLAGNYAPVKRELPLTPCTYTGAIPHELVGGQYVRNGGNPSADADGERDAHWFDGDGMLTGVLFARRHDGSVQPEFINRYVLTDVLMATSASATRPILPSIATLSSPHVTLFRVLLHVVRALWLCLSTFLPWHPAKLAVQRISVANTSIWWHDGKAYAGCESGPPMRILLPGMETAGWWTGAEHDPQSARGWSKAGLAGLFAEMTTAHPHVDPVSGELLLYHMSFFEPFLRVTVIPPRQRAEKAPVCPAPIIGRAVPGLAQPKQMHDFGVSASHTLILDLPLSLDPRNIVRGRPILHYDHLGPTRYGIFPRHAPEAVRWFEEAQACLIFHTANAWDDDAAATVSLLACRLNSATLIYSAGHTVPPPHALPLNGAPESCRLHYWRFTTDPSDASLRPQASFALSAIPFEFPTMNQAYSTRRADFIYGASMRSGSFDAGLGRKSAKIDCLAKIDAGTLLARAAKMSEAGETLGVDDVVDGRTVQELIAAQEAGEGADSAVRIFALPEHHYAQEATFVPRANATAEDDGWLVFFVFDESAGLDADGVALPNAQSELWVLEARGMQDVVARIRLPQRVPYGLHGHFFDAAEIAAQEPVPHEAVRTWARAATGGAASSAIVPAAAASTAVVAAAASAAVVSAAASPAFVAAAAPSALVPVST

SEQ ID NO: 100 - protein, 666 aa, Diaporthe batatas
  REGION 1..666: note = misc_feature - Diaporthe batatas, XP_044637864.1
MSSESEHLIPLAESSPRHPYLSGNFAPIKKTRSLTPCVYSGSIPLDLGGGQYVRNGGNPVSNDDLGRDAHWFDGDGMLSGVLFRRTGEKGSTIQPEFVNQFLLTDVYAYARRRAGPAGELDRSLSRPVLPSIATLVNPALGFFTILWAIVRTIALVLLSHLSRSRYPIKKISVANTSVMYHDGRALALCESGPPLRFALPGLETIGWFNGRTAENEPRNQDDDQKGGFGGGGALSFMREWTTAHPRVDPVTKELIAFHAVFIKPFCYYSIVPPSKRKESLLQPPLSARLSMPVPGVRAPKMMHDFGASRNHTVILDLPLTLDPMNLARGVPVLSYDSRGKSRYGVFPRYRPDLVQWFETNPCVIFHTANCWDSVTHTGEMNGANIQQTSVNLLACRLTSASLVFSAGNLPTPAVKPVPPEYAEEEQCRLYYYNFPLSANDGTDEPGHRIRHQWALSAISFEFPTLSPNHSMSEARYIYGCSTGHASYTVALGKAAKIDYLAKIDVKTLIERGVQHPPQPIRGCVDTRTVVEVQESHDPNDPIKLFKMPDGWFAQEPRFVPRREIERQSEDDGWLLTYVFDESQLGSDGECGEDAVSELWIIDAKTLKDVVARVKLPQRVPYGLHGSWFSEDEINEQRPFTSLRSVADQEGSAFSRSLQGMVERWIG

SEQ ID NO: 101 - protein, 667 aa, Taphrina deformans
  REGION 1..667: note = misc_feature - Taphrina deformans PYCC 5710, CCG82935.1
MEELAISRRLTDNKVHLAAKSTRSRHPYLSGNYWPQRNELHLSPCEVIGSIPQSLAGGQFVRNGPNSSIPPEDGQNYHMFDGDGMIHGVYFKSIPGKDELESLYINRFVHTDVYLASQYWGTSLLPSIASLIDPLTNTVQVLKSILRSGAIAFLSGSARLSTANTALVYHDNRLLATCESGPPMHIGLPELETVDWHTFTDERTGVSLAPIPGSSKEWITGHPKVDPVSGELLMFGYDIFQLFNPHIRFSVIDADGSHKNFQQPVYLKSKTPKMMHDFAITQTRAVILDLPLTMSPFNIVGGGKPMLHFDISLTSKFGVLPRTYDAKRDRDQVVWFESDSCMIFHTVNAWDDHDSAGNVVGVNLVACRFKGAKLVFAAGAIQMPSAENLPSDVVHLYYYRFDLNSGKISHEYPLSDYPIEYPSINTRHLSLPNRYAYGASMREGSFDTALSGAKVDVLVKCDIQALIRSPPTNVTQSSSVTPPSGAHVKTILLPPGLYASEATFVPNTSETATGKEDDGYILVHIFDESTRDPVTKDALDDKGSEVWIISAESFSLPPICKVRLPQRVPYGLHGLWVTKSQIESQRDAKRNSEGDIVAAKASREHTSVRIITNFWKWSLVWAGRSTVGGLSSGRYAASLFIGWTVFILLTYRLFGFIVQHTMQSLSR

SEQ ID NO: 102 - protein, 671 aa, Stereum hirsutum
  REGION 1..671: note = misc_feature - Stereum hirsutum FP-91666 SS1, XP_007305246.1
MKDDSKKSHPYLTGNYSPVRKEWPLTSCQYMGTIPPELYGGQYVRNGANPFHVDKDSWDLNPRDYHWFDGDGMLTGVYFQRLPSTSEASNAQTISPHFVNAYVLTDLYLHARTSPYLRRPLLPSISSFLNPLAGTLGVILSVLRAVFIVVLSHISAFLSRQQSYSVTKISVANTSIVYHDGRALATCESGPPMRVLLPSLTTVGWFDGTHGDGDDVRVNEDPSFGAKPGLLGWMDQWTTGHPRVDPCTHEMILYHSTVLPPYISYTIIPPSSPSTIPFQPAHPRLRNAPIPGVHSPKLSHDFACTRTHTIILDLPLYLDPLNLLSSFLPSRKKNQVMHYQDDEFARFGVFPRYEPHKVRWFEDVEGGACCIFHTVCAWDEMDPRGERVVAVNLLACRLDSAALVYSAGNLATPRTPRDDSTSGLAVDDKEGKCELYYYRFDLSDPDANNITHAFPLSTIPFEFPVVPPSAGMGSATTSSRRFRSPRFVYGCSMQHGTFGAALGGSNARIDAIAKVDVHDLIERGAHRHYDASKSRPAIDQRTVQEILGTADTSVDKGAITIFTFPPGHCGSEPSFCPRANAISEDDGYLIFYVFDERQLERGLSLDEARSELWVVDARNMKDVICRVELPQRVPYGLHAHFFTEDEILGQRGVHSLRSLPAGVEKNDTAGL

SEQ ID NO: 103 - protein, 673 aa, Pseudocercospora fijiensis
  REGION 1..673: note = misc_feature - Pseudocercospora fijiensis CIRAD86, XP_007924069.1
MSTLGKRKRSPSTNIERTPQPKHPYLSGNFAPIQHTLPLTPCTYTGRIPEELADGEYVRNGSNPVSNSDLGRDAHWFDGDGMLSGVSFSKKEDTGEIQPEFVNQFILTDLYLSAKSNGRLRVPILPSIATLVNPLASLMFVTVRILRTILLVILSHLPGSRQKIKKISVANTSVVYHDGRAMAFCESGPPMRIQLPGLETVGWFNGAVAEGEPANESKEKEAVLGENSGLISFMREWTTAHPKVDPQTKEMLMFHSSFAPPYVQYSIIPQTVRAAAEVSPPTRKMLNAGVPGIKSAKMMHDFGVSSAHTIIMDLPLSLDPLNQLKGLPPVVYNPDRPSRFGVFPRRHPKQTKWFETSACCIFHTANTWDEVGLDGSTTSVSMLACRLTSATLIYAAGNMAPPPLLRVPSLEEHLLKDPFAEEQCRLYYYNFDLDSGSISHQWALSAVPFEFPSVRPDLEMSQARFVYGCSTTTTDFGSALGKATKIDALVKIDATKLIERGRAHPPKSVTGVVDGRTVSQVLISRDPRDPIKVFRMPEGWFAQEPRFVPRERSAHERQLNDEPLDEDDGYLLFYAFDETQLLANGEVPDDSAKDQRAKSELWIVNAKNMRDVIARVHLPQRVPYGLHGSWFSAEQISEQRPVDTIRSMEEVMKEKNDNGLWMKVRERIETLLG

SEQ ID NO: 104 - protein, 682 aa, Meira miltonrushii
  REGION 1..682: note = misc_feature - Meira miltonrushii, XP_025354342.1
MTGSSSSSTKSRSSSSASSETSIIPFPKKTLHPYLTGNFAPIKRELPLTQCKVAYGKVPEEVCGGQYIRNGGNPFKDEDQDRQAHWFDGDGMLTGVLFQSDGEGIRPHFINRYILTDVLLSTSQASRKPFLPSISTFASQLSFFGFFILMGEIMRTFVLAMLSFLPFQSRPSVKRISAANTSIWWHDGQAFAGCESGPPIRVLLPGLETAAWWTGEEETEGGWSKGGMGPIKMLKEFTTAHPRIDPETDELLLYHMSFAAPYLRISVVPTRKSIEAGAKPLMGAAVPGLNKPKLAHDFCATSKRTILIDMPLVLDPRNLLQGKPMLSYEENMPTRFGILPRRSPEKVKWYESEGCCIYHAANAWDDEATSSTNLVACRLNSATLVYAAGNLDTPSHAKATCGTEKCQLYYWQFADAPEDNQAVPEKNEKAQVLREFALSDVPVEFPTMNDSYSQQCNRFVYGASMTTGSFDAGLGSRSAKIDCLAKFDIQTLIAKGKKAMDAGQLQKGEAVDKRTVQDVLAAEQTEDDPIRLFALPPHHYAQEATFVPRLNAKSEDDGFLLFFVFDESTGLDYDGDALPNAISQLWILDAKTMKDVICKICLPQRVPYGLHGHFFSATEIQNQRPVDTDQVRNWALAQTAQTGIGLGGVKASSDHDNNLSSFTAFAYSAVANFRNSIEHFLG

SEQ ID NO: 105 - protein, 682 aa, Kwoniella heveanensis
  REGION 1..682: note = misc_feature - Kwoniella heveanensis CBS 569, OCF41229.1
MTKTLRAVPDGGHIHPFLKGNFAPVTDEYISHPCEVLEGEIPDELLGGQYIRNGGNPVYPPEQGRHYHWFDGDGMLHGVYFPAEKGAEPLYTNRHLATPLMTMTLLLLRSPIPSIALLISPLSSLHRIVMAIVQSFFIAVRARMGVLSVANTSDAVRSQRLLATCESGPPLEVRVPDLQTVDWDRLEEESTGENLGQRRGQWEWWKRIGLSRVQEDWMTAHPRIDPVDGSLIFYSTQMFDAPHVRYSVIDRKGRHVVWKEGVDVGRAKMMHDFAATRTHTVLLNLPLTLAPHNLFQRHPVPLIHFDRSLPSEFVILPRLYRSHDTPQHPIRFRESEPSLIFHTANAWDEYDREGDLVAINMLGCRFKSAKLVYAAGQVDVPLEEQKFGEDDVVRLHYHQFDMRGYTTSAEPPTEGEITYSFPLSAMPFEFPCTPTHLGMSAARYVYGCTMKSGSFDERLGGAAKVDCIAKLDVLELISRGQRRGKGKNILPVDERSSAQILEDAQRGIKGPIQIFAFPEGCYAQETQFIPRSDPKSEDDGFLVTYVYDEKYLQADGTPSPALGAGSELWIIDAKRLAEGTSGVVAKVKLPQRVPYGLHGTFVPASLIRNQRVLPQPLADQPLLQDKLARSRLQYFVSILFDRPATRKRSHFERGVLWVLWPAAVLMSFLSLWTASKHLFWLR

SEQ ID NO: 106 - protein, 685 aa, Epichloe festucae
  REGION 1..685: note = misc_feature - Epichloe festucae Fl1, QPG95396.1
MYHPSEALSSVSAVTLALFICVTITSSILTWTWLNKDKDKKERTVPHPYLQGNFAPIQTTRPLTPCKYTGTLPRELRGGQYVRNGGNPVTNDHLGRDAHWFDGDGMLSGVAFIESDSDDAVVPHFVNQFILTDVYLSLSSMTTPLRTPILPSIATLIDPLSTLYTITSSILRTVSLVVLSHLPGSLHPIKKISVANTGVLYHDGRALATCESGPPMRIALPTLDTIGWFNGHHAEGEGQGQGQGEENHEPMDTGMEAGTGADAVVIGGTGPLSFMREWTTAHPRSDPVTGELLLFHSTFIAPFVRYSVIPAEQDSRKTLPRPRLLNAAVPGISSPKMMHDFGVSPTHTIIMDLPLSLAPQNLARNLPIVSYDPSSPSRFGIFPRYSPQTVRWFHTDPCCIFHTANTWDSPDAVHLLACRLTSPSLIFSAGGIPVAPAKDDQCRLYYYQFPRDASSQSITHEWALLSLPFEFPSVPDAMSMSPARYIYGCSSASNFSAALGRAVKIDALVRVDVQQLIARGIDDPPAARVTGCVDTRSLDEIASSSDPNDPVRVFQLPAGWYAQEPRFVPRDGGSEEDGWLLTYVFDESQMDEHGNVGVDAVSELWIIDAPRMSAGMRAVVARVRLPQRVPYGLHGSWFSREEVDGQRPWNHSRSSELRETRKLGREVHEGLWGRARDVLISSCLA

SEQ ID NO: 107 - protein, 688 aa, Pseudomicrostroma glucosiphilum
  REGION 1..688: note = misc_feature - Pseudomicrostroma glucosiphilum, XP_025348787.1
MTRRTWALAQLNEKHGYLAGNYAPVEEETPLTPCMVVSGSIPNELAGGQYVRNGGNPMANDDAGRESHWFDGDGMLSGVLFQRRANGSIAPHFVNRYVVTDILLATRQGAHRPILPSIATLSSPSVSLLRVLYEVMRSLVLCLLTWIPLTWSIGLGKGTRRLSKISVANTNIFYHDGKALAACESGPPMRIMLPKLETGGWWTGDEGKSWASGIGPLKIFNEMTTAHPHTDTSTNELLLYHSTFVAPYLRISVIPPRSSRRSALIGATVPGMKEGKLMHDFAATKTRTVVMDLPLTLDPRNLLSGKPVVHYEPTAAVRFGVFPRREPEKVRWYEDPTACCIYHTANCWDESQAETSQSGSSEDAVNFLACRLNSATLIYSAGNTLAPTTAKAPNGEEERCELHYWRFAEPPKATELLPPLDSTALEKAPKAQHDSPARITHSFPLSRVPFEFPTINLAHAPAAGLGPNRFVYGCSMREGTFDAGMGRHSAKIDCLVKMDVQSLVKRGRARADAGKMKKGEAVDMRSMPEIIASAEHAQDGAVQGDITVFPLPHGWYAQETTFIPRSNPQGEDDGFLLFFCFDELTHLHPATGEPLPSAVSQLWVLDARDMKTVVARIELPARVPYGLHGKWFTEAEIDGQERVDEQSIRRWALRDVQKSDRRSKEERIKREGKLNVLNALRRWVELWA

SEQ ID NO: 108 - protein, 691 aa, Fusarium tricinctum
  REGION 1..691: note = misc_feature - Fusarium tricinctum, KAH7251169.1
MSHSNEKTAQPLRHPYLIGNFAPIQKTTSLTPCTYSGRIPPELTGGQYVRNGGNPVSHEDLGRDAHWFDGDGMLSGVAFRKMSPDGRVVPEFVNQYIFTDLYLSRKTTSVKSAIMPSITTLVNPLSTMMRIVLATFRTMFLVLLSNLPGSQQSLKRTSVANTALVYHDGRALATCESGPPMRIQLPSLDTVGWYNGVEAEGESKDPTLNENDSPFGGDGLFKSMKEWTTGHPKVDPISGEMILYHNTFLRPYVHYSVLPQRDEKTCSGSKLVNLPVPGVSGARMMHDFGASRAHTIVMDLPLSLDPLNLFKGKEVVSYDSTKPSRFGVFPRHDPSSVRWFHTAPCCIFHTANTWDTLSSDGNSSVNLLACRMTSSTVVYTAGNILPPVVTKTSGVRVKGSKQDVDRWDVDGDAACRYEEAPLLESPGEKAGSADYFSSSDESDDYAQCRLYYYEFDMSTKSQNKVKHQWALSAVPFEFPSVRPDRQMQQARYIYGCSTSASSFSVALGRADKIDLLVKMDAKTLIEKGKKTKTTSITGCVDRRTVSEIPYEQEDDDPVRIFRLPPNHYAQEPRFIPRSSSTEEDAGYLLFYVFDESQILPSGDCPPSSVSELWVLDAQNMRDVVAKVKLPQRVPYGLHGTWFSSSDIEEQRGVESFRNLEVMQKKKEGWVNGGFVKRSWMTVREKLEKSVG

SEQ ID NO: 109 - protein, 695 aa, Fusarium bulbicola
  REGION 1..695: note = misc_feature - Fusarium bulbicola, KAF5980194.1
MKFLQRYSFTQTSMPRPDKNISPPLRHPYLTGNFAPIHQTTNLTPCTYSGCIPSELTGGQYVRNGGNPVSHQDHGKDAHWFDGDGMLSGVAFRKKSMDGRITPEFVNQYILTDLYLSRKTTSIVSPIMPSITTLVNPLSTMFTIMFATFRTIFLVILSNLPGSQQAIKRISVANTAVLYHDGRALATCESGPPMRIQLPSLDTVGWFDGVKAEGEPEKSQASSNDSPFGGSGLFSFMKEWTTGHPKVDPVTGEMLLYHNTFMPPYVHYSVIPKSNEKASGHRLVNQSVLGVSGARMMHDFGASRSHTIIMDLPLSLDPLNTMKGKEVVSYDPTKPSRFGVFPRHQPPSVRWYRTAPCCIFHTANTWDSHSSDQGSSVNLLACRMTSSTLVYTAGNIRPPVKSKSTPARDWSDDKKETTCRYKEAPTLESPGVAAGLTDYFPTAESDDYDQCRLYYYEFDTSTASQNEVKNQWALSSIPFEFPSVRPDREMQDARYIYGCSTSTSCFGVALGRADKVDLLVKMDAKTLVRRGKKLNTTPVTGCVDRRSVSEILEQQKKDDPIKIFRLPPNHYAQEPRFVPRASSSEEDDGYLLFYVFDESQLLPTGDCPPSAKSELWILDAKNMRDVVAKVRLPQRVPYGLHGTWFSSRDIEGQRAVESLRSLEEVQRKKEDWVNGGGQIRKSWMVLREKLERAVG

SEQ ID NO: 110 - protein, 696 aa, Fusarium fujikuroi
  REGION 1..696: note = misc_feature - Fusarium fujikuroi, SCV49259.1
MKFLQQNSFTQTSMSQPHEDVSPPLRHPYLTGNFAPIHKTTNLTPCTYSGCIPPELTGGQYVRNGGNPVSHQDLGKDAHWFDGDGMLSGVAFRKASIDGKTIPEFVNQYILTDLYLSRKTTSIASPIMPSITTLVNPLSTMFQIMFATFRTIFLVILSNLPGSQQAIKRISVANTAVLYHDGRALATCESGPPMRIQLPSLDTVGWFDGVEAEGEPEISQAGSDDSPFGGSGIFSFMKEWTTGHPKVDPVTGEMLLYHNTFMPPYVHCSVLPKSNEKAPGHRLVNQPVLGVSGARMMHDFGASRSHTIIMDLPLSLDPLNTMKGKEVVAYDPTKPSRFGVFPRHLPSSVRWFHTAPCCIFHTANTWDSQSSEGELSVNLLACRMTSSTLVYTAGNIRPPVRSRCTQARVWSDEKEETACRYKEAPALESPGESTGLVDYFPITAESDDYDQCRLYYYEFDLTMESRNHVKSQWALSAIPFEFPSVRPDREMQEARYIYGCSTSTSCFGVALGRADKVDLLVKMDAKTLIQRGKKMNATSITGCVDRRSVCEILQEQRKDDPINIFRLPPNHYAQEPRFVPRACSTDEDDGYLLFYVFDESQLLPSGDCPPSATSELWILDAKNMRDVVAKVRLPQRVPYGLHGTWFSSQDIESQRSVKSLRSLEVVQRKKEEWVNGGGQIRKSWMVLREKLEKAVG

SEQ ID NO: 111 - protein, 701 aa, Clavulina sp.
  REGION 1..701: note = misc_feature - Clavulina sp. PMI_390, KAF8309329.1
MKRTNIPHQKPTSVLPHPYLSGNFAPVSIELPLTECTVIGTIPSELAGGQYVRNGANPLANDDGTRDAHWFDGDGMLAGVFFCQDHSGAIRPHFVNRFVVTDVFAASAGRKTPLAHPFVISISTMSSILRSPVSILIAILRTFILAVLSWLPGLAPFPVKRISVANTAILYHDGRALATCESGPPMRVLLPGLETIGWWSNATESGSTKGHAARGLARFMREWTTGHPKVDPISGEFIAFHNTVIRPYISYSVIPPSRLSAAGRSSTTAHSQGAENGLQPSSLISLAGAPIQPSPSSPKMSHDFGVSLTHTVLVDPPLLLDPLNLLRGKPILTYDTSPEAKLRFGVFKRYSPHDVHWVDTHPGVVLHVANTWDEYGDEVLADGDKNVVGINMLVPRMNSASLVYATGNIPSPHASLQQDEETDEEGEPEECTLHYFYFSLPSSGTLAPHNTESDTPPTSSTSHLASLPTSRASSTTLLHSFPISAIPLELPTVSPHTAMRCARYIYGCTMREGTFSGGKVQCLAKFDVLTLIDRGKRESVSDGIERRPVDQRSVQEIIQQQLEAQQSESAIKIFNMPPGWCCQEATFVPRTNQQSYSLPASSDEEEDDGWLLSYVFDESQLDGAGMPRADARSELWIIDARNMKDVVARIVLPQRVPYGFHGSFFTEDQIAQQQPIDDIRHSPTQPCRHHFLHQFTPPLIC

SEQ ID NO: 112 - protein, 701 aa, Neohortaea acidophila
  REGION 1..701: note = misc_feature - Neohortaea acidophila, XP_033588727.1
MPLAGQKRKRAASGDILSTPQPKHPYLTGNFAPIHNTLPLTPCSHTGTIPPELSDGQYVRNGSNPVTNEDLGRDAHWFDGDGMLSGVSFRKDDTGIVPEFVNQFILTDLYLSALSSPRLKVPILPSIATLVHPLYSIFYVTLRIFRTVLLVLLSHLPGSKQKIRKISVANTNVLYHDGRALALCESGPPMRVQLPELETVGWYNGDRAQGEPEKERGAALGDGSGLLQFMREWTTAHPKVDPQTKEMLMFHSSFAPPYVQYSIIPKEKSPSDEAVASQKLLNAPVPGVRKAKMMHDFGVSLTHTVIMDLPLTLDPVNQLKGLPPVTYDASQPSRFGVFPRRSPEQVQWFETDAACIYHTANTWDDVNATGSPTAVNMLACRLTSATLIYAAANIAPPAPKSNKRVAQANRRMPFFAKYNEEEDPSLWERAPLLESPSEEKDTFIRVDALESPSSNPAAFDEEQCRLYYYHFDLNNGALTHQWALAAIPFEFPSVRPDLEMQHARYVYGCSTTTTSFSSALGKATKIDALVKVDVNRLIANGKSHPPQSVTGVVDPRPMAAILSANDPHDPIQVFRMPPGWFAQEPRFVPAASNASEDDGYLLFYAFDEAQLNAAGDAPDDVSDSRAKSELWIIDAKTMKDVVARVQLPQRVPYGLHGTWFSAEQIRAQRPAEQLRSTREVLGAKEVGMWMGVRDWCEKMLG

SEQ ID NO: 113 - protein, 702 aa, Peltaster fructicola
  REGION 1..702: note = misc_feature - Peltaster fructicola, QIW99276.1
MAFTSQKRKRAVSELMQPTPQPKHPYKSGNFAAIHQTLPLTPCTYTGTIPKELGDGEYVRNGSNPLGSGSQEDIGRDYHWFDGDGMLAGVWFERDASGEISPQFVNQYILTDVYLSAIGSRHLKKPILPSIATLVDPLASFFWVTIQIFRTLLLVLLSHLPGSKQKIKKISVANTHIMYHDGRALALCESGPPMRIQLPGLETVGWFNGSVAKGEPLKTITEKEPVLGGDDSLYSFMREWTTAHPKVDPKTKEMLAFHSCFAPPYIQYSILPESSTVEKQKMLNAPVPGIRGARMMHDFGASSAHTIIMDLPLSLDPTEQFKGRPTVNFDATQSSRFGVFPRRRPEDVRWFETDACCIFHTANSWDSLTMGKTTGVNMLACRLTSATLVFAAGNIAPPAERKKVKADIIKKRRMPFFAKYDQSIDVTAWKDSEQLGTPEDEKQAFIRIVPEPIAGSGPGDYDDEQCRLYYYHFDLETEAIAHQWALSAISFEFPSVNPQYEMAAARYVYGCSTTDESFGQALGKATKIDVVVKMDVQTLIARGKKQPPASVTGVVDERLIEEVLEAGSDDPIQAFKMPDGWYAQEPRFVSASNPTSEDDGFLIWFAFDESQLLDNGDVPADNTTQRAKSELWIVNAKDMRTVLARVQLPQRVPYGLHASWFEQSQIQQQRPVAEVRSTAIALSPNPPGLWMEIRDWVEACLG

SEQ ID NO: 114 - protein, 703 aa, Lizonia empirigonia
  REGION 1..703: note = misc_feature - Lizonia empirigonia, KAF1365755.1
MSRHGHSKKHTHKHPYLGGNFAPVTRTWTPTSVTWTGNLPKELNGGMYVRNGGNPIANSDLGREAHWFDGDGMLSGVWFGRSTNDANELHVQFVNQFVLTDVYLSSLENNTLRTPILPSIATLTNPASSLIWLIWRIMRTVLIVLLSHLPGSKFAIKRISVANTSVLYHDGRALATCESGPPLRLTLPQLETVGWFDGNAVEGEPKTEDKKEEMTLGGTGPLSWMREWSTGHPKVDNATGEMVLFHCSFAPPYVQYSLIPPTGSNITDHQSADDAGRIINAAVPGCTGGKLMHDFGVSKHSTVILDLPLSLTPLNLAKNSPVVAYEPHKPSRYGIFPRQHPEAVRWFETEGCCIFHTANTWDEYDSDGNVAAVSLLACRLTSASVIFSAGNIAPPPHPKHKDAISEKPMSFFAKYDSDADDQDPEKILSDETSPLLTYRDAVPNPFGASSSLPISNDDEEQCRLYYYRFSLESMTENVITHQFALAAIPVEFPTVSPLTAMMSARYIYACSTSNESFGAALGKATKIDVIVRFDVQALLARAQIEPPDAITGCVDNRTLGQIMASNDPNDPIKAFRLPEKHYGQEATFIPRSSRGSATNKNTAFDEDDGYLVFYVFDEHQLDEAGECKENARSELWVLDAQTMNRVECKIQLPTRIPYGLHGNWFTEEQIARQKGVEKVRALPAVAPVTKWSRIKRSIHGRLG

SEQ ID NO: 115 - protein, 703 aa, Cercospora beticola
  REGION 1..703: note = misc_feature - Cercospora beticola, XP_023459336.1
MFQLTSEGGGSRFITNPLAGQKRKRSPSTNIQPTPQAKHPYLSGNFAPIQQTLPLTPCTYTGSIPEELAEGEYIRNGSNPVSNDDLGRDSHWFDGDGMLSGVSFTKDEQTGEVTPEFVNQFVLTDLYLSTLGSDRLRVPILPSIATLVSPVASFIWVTIRILRTVLLVLLSHLPGSKQKIKKISVANTNVIYHDGRALATCESGPPMRIQLPGLETVGWYNGVSAEGEPVEEREEQGKDKAFGQDGGLISFMREWTTAHPKVDPVSKEMLLYHASFAPPYVQYSVIPKASSTTGTKGKRLVNVGIPKVSGAKMMHDFGVSSQHTIIMDLPLTLDPINQLKGLPTVHYDSSKPSRFGVFPRHSPAETQWFETDGCCIFHTANSWDTVESDGHVSSVNMLACRLTSATLIYAAGNMAPPPLKPLTSSVAKKTKRMPFFSSYDTGNGHTNFDPDQPLDEKEPLLRVGERLYYYSFDLNSTNITHQWALSAVPFEFPSVHPGREMSEAQYVYGCSTTTTDFGAALGKATKIDAIVKIDAKKLIQRGRANPPKSVTGVVDIRTMAQVLETSDPEDSIRVFVMPPGWFAQEPRFVPRQNPASEDDGYLLFYAFDEGQLTPDGDVPADNCEGRAKSELWIVDAKNMQKVVGRVQLPQRVPYGLHGSWFSAEMIKNQRAVESIRSTAKALERDGAGGVWMKVRDTLEGWLG

SEQ ID NO: 116 - protein, 704 aa, Fusarium sporotrichioides
  REGION 1..704: note = misc_feature - Fusarium sporotrichioides, RGP69130.1
MDSLQHIVPAVVSISHPKEQSPPRTLRHPYLIGNFAPIHKTLNLTPCTHSGCIPPELTDGQYVRNGGNPVSHEDLGRDAHWFDGDGMLSGVLFRKGHSDGQIVPEFVNQYILTDLYLSRKTTSVSSPIMPSITTLVNPLSSLLKILLATFRTMFLVFLSNLPGSQQALKRTSVANTAILYHDGRALATCESGPPMRIQLPSLDTVGWYNGAHAEGEPEQALSENKIEPFGGDSLFKSMREWTTGHPKVDPVSGEMILYHNTFIQPYVHYSVLPKTDVQAPTTGRLVNQAVPGVSGARMMHDFGASRAHTIIMDLPLTLDPMNLAKNKEVVSYDPSKPSRFGVFPRHEPSNVRWFQTAPSCIFHTANSWDTKSVNGTSSVNLLACRMTSSTVVYTAGNIKPPVKPKRHNNRLPHAKENISQWDEKDIQRFEAAPMLESPTEKLYGDDYFSSSDEADDYSQCRLYYYEFDMSATSTNNVINQWALSTIPFEFPSVRPDREMQDARYIYGCSTSTSCFGVALGRADKVDLLVKMDAKTLVERGKRMNTRPVTGCVDRRSAREILDSQDENDPIKIFRLPPRHFAQEPRFVPRAGVAEEDAGYLLFYVFDESQILPNGDCPSSSASELWILDAQNMRDVVAKVRLPQRVPYGLHGTWFSARDIEEQRAIETLRSLEAVQRKKEIWVNDGGSIARSWMAFREKLERAVG

SEQ ID NO: 117 - protein, 703 aa, Ramularia collo-cygni
  REGION 1..703: note = misc_feature - Ramularia collo-cygni, XP_023621334.1
MQEMAHAGQKRKRTPTPHIPPTPQAKHPYLAGNFAPISQTLPLTKCTWTGHIPQELADGQYVRNGSNPVSNEDLGRDAHWFDGDGMLSGVLFRRDTTSGDITPEFVNAYVLTDVHLSATSSPRLIRPILPSIATLVDPLASFIYVTWRIIRTILLVILSHLPGSKQAIKKISVANTNIVFHDGRALATCESGPPIRIQLPGLETVGWYNGSTAQNEPPQPLPSAEECRFLGQDSGLIGFMREWTTAHPKVDPVTQEMLMFHSSFAPPYVQYSIVPAQQTSSTSHLPRRKMLNAAVPGVTSAKMMHDFGCSSTHTVIMDLPLSLDPLNTFKGTPTVAYDPSKPSRFGVFPRHSPQQVTWYETDASIVMHTANTWDEVDETTRQVTGVNMLCCRLTSATLVFAAGNIAAPAPIPRKPAVVAKRKRMSFFSPYNAAVEPTHVERHVDEKEPLLHTVENTNLDDEEELMEEDQCRLYYFHFSLTNPSTIISQYALTAIPFEFPSTHPAKSMQSARYIYGCSTTTSSAFGAALGKATKIDAVVKVDAGTLIERGRRNPPKSVSGVVDSRPVEDISVGDEVIGMFKLPPGWFAQEPRFVPRDGGQGEDDGWLLFYAFDESQLDATTGEVEREEGVHSELWILDARDMDSVVARVVLPQRVPYGLHGSWFPKEEIEGQRGVVEFRREIQGTEEGRGVWAAVRRGLERGLA

SEQ ID NO: 118 - protein, 705 aa, Zymoseptoria tritici
  REGION 1..705: note = misc_feature - Zymoseptoria tritici ST99CH_3D1, SMR64689.1
MVHAGQKRKRSPSENIPPTPQPRHPYLTGNFAPIQQTLPLTPCTYTGQIPEELADGEYVRNGGNPVSNDDLGRDAHWFDGDGMLSGVAFTKDEATGRIQPEFVNQFILTDVYLNTLASPKLRVPILPSIATLVNPLASFIWVTIRIFRTILLVILSHLPGSKQAIKKISVANTNVVYHDGRALATCESGPPIRIQLPGLETVGWYDGANAQGEPMKEDTAKEATLGQNSGLISFMREWTTAHPKVDPTTKEMLMFHSSFAPPYVQYSIVPKSSESEGGEGRTRRSKILNAAVPGVTSAKMMHDFGVSPTHTVIMDLPLSLDPINQLKGLPTVTYTPSKPSRFGVFPRRQPESVQWFETDASCIFHTANTWDDVSSAGETTAVNMLACRLTSATLVFAAGNIAPPIEKLKAPPVKAKRRMPFFSKYDSATEVTIHDLDSPTDEKEPLLTVTTVDEAGEVEEPFEEDQCRLYYYHFDMTTHNITHQWALSSIPFEFPSVRPDVEMQNAQYIYGCSTTTTSFGSALGKATKIDALVKIDAKTLIERGRRTPPPSVTGVVDPRNMTQVLADQHDPTDPVRVFTMPEGWFAQEPRFVPSSSNASEDDGYLLFYAFDEGQLSSLGDVPDDVSPSRAKSELWIVNAKNMTDIVARVQLPQRVPYGLHGSWFTAEQIAEQKPVECIRTTAKALEVREKGVWMSVRSWVEGVLG

SEQ ID NO: 119 - protein, 705 aa, Zymoseptoria tritici
  REGION 1..705: note = misc_feature - Zymoseptoria tritici ST99CH_1A5, SMY30021.1
MVHAGQKRKRSPSENIPPTPQPRHPYLSGNFAPIQQTLPLTPCTYTGQLPEELADGEYVRNGGNPVSNDDLGRDAHWFDGDGMLSGVAFTKDEATGRIQPEFVNQFILTDVYLNTLASPKLRVPILPSIATLVNPLASFIWVTIRIFRTILLVILSHLPGSKQAIKKISVANTNVVYHDGRALATCESGPPIRIQLPGLETVGWYDGANAQGEPKKENTAKVVTLGQNSGLISFMREWTTAHPKVDPTTKEMLMFHSSFAPPYVQYSIVPKSSESEGGEGRTRRSKILNAAVPGVTSAKMMHDFGVSPTHTVIMDLPLSLDPINQLKGLPTVTYTPSKPSRFGVFPRRQPESVQWFETDASCIFHTANTWDDVSSAGETTAVNMLACRLTSATLVFAAGNIAPPIEKLKAPPVKAKRRMPFFSKYDSATEVTIHDLDSPTDEKEPLLTVTTVDEAGEVEEPFEEDQCRLYYYHFDMTTHNITHQWALSSIPFEFPSVRPDVEMQNAQYIYGCSTTTTSFGSALGKATKIDALVKIDAKTLIERGRRTPPPSVTGVVDSRNMTQVLADQHDPTDPIRVFTMPEGWFAQEPRFVPSSSNASEDDGYLLFYAFDEGQLSSLGDVPDDVSPSRAKSELWIVNAKNMTDIVARVQLPQRVPYGLHGSWFTAEQIAEQKPVECIRTTAKALEVREKGVWMSVRSWVEGVLG

SEQ ID NO: 120 - protein, 705 aa, Zymoseptoria tritici
  REGION 1..705: note = misc_feature - Zymoseptoria tritici, Speckled leaf blotch fungus; Septoria tritici , UPI001309FD1A
MVHAGQKRKRSPSENIPPTPQPRHPYLTGNFAPIQQTLALTPCTYTGQIPEELADGEYVRNGGNPVSNDDLGRDAHWFDGDGMLSGVAFTKDEATGRIQPEFVNQFILTDVYLNTLASPKLRVPILPSIATLVNPLASFIWVTIRIFRTILLVILSHLPGSKQAIKKISVANTNVVYHDGRALATCESGPPMRIQLPGLETVGWYDGANAQGEPKKEDIAKEATLGQDSGLISFMREWTTAHPKVDPTTKEMLMFHSSFAPPYVQYSIVPKSSESEGGEGQTRRSKILNAAVPGVTSAKMMHDFGVSPTHTVIMDLPLSLDPINQLKGLPTVTYTPSKPSRFGVFPRRQPESVQWFETDASCIFHTANTWDDVSSAGETTAVNMLACRLTSATLVFAAGNIAPPIEKLKAPPVKAKRRMPFFSKYDSATEVTIHDLDSPTDEKEPLLTVTTVDEAGEVEELFEEDQCRLYYYHFDMTTHNITHQWALSSIPFEFPSVRPDVEMQNAQYIYGCSTTTTSFGSALGKATKIDALVKIDAKTLIERGRRTPPPSVTGVVDPRNMTQVLADQHDPTDPVRVFTMPEGWFAQEPRFVPSSSNASEDDGYLLFYTFDEGQLSSLGDVPDDVSPSRAKSELWIVNAKNMKDVVARVQLPQRVPYGLHGSWFTAEQIAEQKSVECIRTTAKALEAREKGVWMSVRSWVEGVLG

SEQ ID NO: 121 - protein, 705 aa, Zymoseptoria tritici
  REGION 1..705: note = misc_feature - Zymoseptoria tritici, Speckled leaf blotch fungus; Septoria tritici , UPI00130ABF23
MVHAGQKRKRSPSENIPPTPQPRHPYLTGNFAPIQQTLPLTPCTYTGQIPEELADGEYVRNGGNPVSNDDLGRDAHWFDGDGMLSGVAFTKDEATGRIHPEFVNQFILTDVYLNTLASPKLRVPILPSIATLVNPLASFIWVTIRIFRTILLVILSHLPGSKQAIKKISVANTNVVYHDGRALATCESGPPMRIQLPGLETVGWYDGANAQGEPKKENTAKVVTLGQDSGLISFMREWTTAHPKVDPTTKEMLMFHSSFAPPYVQYSIVPKSSECEGGEGQTRRSKILNAAVPGVTSAKMMHDFGVSPTHTVIMDLPLSLDPINQLKGLPTVTYTPSKPSRFGVFPRRQPESVQWFETDASCIFHTANTWDDVSSAGETTAVNMLACRLTSATLVFAAGNIAPPIEKLKAPPVKAKRRMPFFSKYDSATEVTIHDLDSPTDEKEPLLTVTTVDEAGEVEEPFEEDQCRLYYYHFDMTTHNITHQWALSSIPFEFPSVRPDVEMQNAQYIYGCSTTTTSFGSALGKATKIDALVKIDAKTLIERGRRTPPPSVTGVVDPRNMTQVLADQHDPTDPVRVFTMPEGWFAQEPRFVPSSSNASEDDGYLLFYAFDEGQLSSLGDVPDDVSPSRAKSELWIVNAKNMKDVVARVQLPQRVPYGLHGSWFTAEQIAEQKSVECIRTTAKALEAREKGVWMSVRSWVEGVLG

SEQ ID NO: 122 - protein, 705 aa, Zymoseptoria tritici
  REGION 1..705: note = misc_feature - Zymoseptoria tritici, Speckled leaf blotch fungus; Septoria tritici , UPI00130CFB6E
MVHAGQKRKRSPSENIPPTPQPRHPYLTGNFAPIQQTLPLTPCTYTGQIPEELADGEYVRNGGNPVSNDDLGRDAHWFDGDGMLSGVAFTKDEATGRIHPEFVNQFILTDVYLNTLASPKLRVPILPSIATLVNPLASFIWVTIRIFRTILLVILSHLPGSKQAIKKISVANTNVVYHDGRALATCESGPPMRIQLPGLETVGWYDGANAQGEPKKENTAKVVTLGQDSGLISFMREWTTAHPKVDPTTKEMLMFHSSFAPPYVQYSIVPKSSECEGGEGQTRRSKILNAAVPGVTSAKMMHDFGVSPTHTVIMDLPLSLDPINQLKGLPTVTYTPSKPSRFGVFPRRQPESVQWFETDASCIFHTANTWDDVSSAGETTAVNMLACRLTSATLVFAAGNIAPPIEKLKAPPVKAKRRMPFFSKYDSATEVTIHDLDSPTDEKEPLLTVTTVNEAGEVEELFEEDQCRLYYYHFDMTTHNITHQWALSSIPFEFPSVRPDVEMQNAQYIYGCSTTTTSFGSALGKATKIDALVKIDAKTLIERGRRTPPPSVTGVVDSRNMTQVLADQHDPTDPIRVFTMPEGWFAQEPRFVPSSSNASEDDGYLLFYAFDEGQLSSLGDVPDDVSPSRAKSELWIVNAKNMTDIVARVQLPQRVPYGLHGSWFTAEQIAEQKPVECIRTTAKALEVREKGVWMSVRSWVEGVLG

SEQ ID NO: 123 - protein, 705 aa, Zymoseptoria tritici
  REGION 1..705: note = misc_feature - Zymoseptoria tritici, Speckled leaf blotch fungus; Septoria tritici , UPI0013063113
MVHAGQKRKRSPSENIPPTPQPRHPYLSGNFAPIQQTLPLTPCTYTGQLPEELADGEYVRNGGNPVSNDDLGRDAHWFDGDGMLSGVAFTKDEATGRIQPEFVNQFILTDVYLNTLASPKLRVPILPSIATLVNPLASFIWVTIRIFRTILLVILSHLPGSKQAIKKISVANTNVVYHDGRALATCESGPPIRIQLPGLETVGWYDGANAQGEPMKEDTAKEATLGQNSGLISFMREWTTAHPKVDPTTKEMLMFHSSFAPPYVQYSIVPKSSECEGGEGQTRRSKILNAAVPGVTSAKMMHDFGVSPTHTVIMDLPLSLDPINQLKGLPTVTYTPSKPSRFGVFPRRQPESVQWFETDASCIFHTANTWDDVSSAGETTAVNMLACRLTSATLVFAAGNIAPPIEKLKAPPVKAKRRMPFFSKYDSATEVTIHDLDSPTDEKEPLLTVTTVNEAGEVEELFEEDQCRLYYYHFDMTTHNITHQWALSSIPFEFPSVRPDVEMQNAQYIYGCSTTTTSFGSALGKATKIDALVKIDAKTLIERGRRTPPPSVTGVVDSRNMTQVLADQHDPTDPIRVFTMPEGWFAQEPRFVPSSSNASEDDGYLLFYAFDEGQLSSLGDVPDDVSPSRAKSELWIVNAKNMTDIVARVQLPQRVPYGLHGSWFTAEQIAEQKPVECIRTTAKALEVREKGVWMSVRSWVEGVLG

SEQ ID NO: 124 - protein, 706 aa, Zymoseptoria brevis
  REGION 1..706: note = misc_feature - Zymoseptoria brevis, KJY02167.1
MVHAGQKRKRSPSENIPPTPQPRHPYLTGNFAPIQQTLPLTPCTYTGQIPEELADGEYVRNGGNPVSNDDLGRDAHWFDGDGMLSGVAFTKDEATGRIQPEFVNQFILTDVYLNTLASPKLRVPILPSIATLVNPLASFIWVTIRIFRTILLVILSHLPGSKQAIKKISVANTNVVYHDGRVLATCESGPPMRIQLPGLETVGWYDGANAQGEPVNEDTAKEEATLGQDSGLISFMREWTTAHPKVDPTTKEMLMFHSSFAPPYVQYSIVPKSSESGGVEGQTRRNKILNVAVPGVTSAKMMHDFGVSPTHTVIMDLPLSLDPINQLKGLPTVTYTPSKPSRFGVFPRRQPESVQWFETDASCIFHTANTWDDVSSAGETTAVNMLACRLTSATLVFAAGNIAPPMEKLKAPPVKAKRRMPFFSKYDSATEVTIHDLDSPTDEKEPLLTFTTLDEAGEVEELFEEDQCRLYYYHFDMTTHNITHQWALSSIPFEFPSVRPDVEMQNAQYIYGCSTTTTSFGSALGKATKIDALVKIDARTLIERGRRTPPPSVTGVVDPRNMTQVLAGQHNPTDPIRVFTMPEGWFAQEPRFVPSSSNASEDDGYLLFYAFDEGQLSSLGDVPDDVSPSRAKSELWIVNAKNMTDIVARVQLPQRVPYGLHGSWFTAEQIAQQKPVECIRTTAKALEVREKGIWMSARSWVEGVLG

SEQ ID NO: 125 - protein, 707 aa, Fusarium solani
  REGION 1..707: note = misc_feature - Fusarium solani, KAH7254831.1
MSVVVDVSTSGFVRHCETLYWSSTAKLAPFSITGGLGIEWVWSCYLSGNFAPVQQTTPLTPCVYTGNIPPELQGGQYVRNGGNPVGHEDLGRDAHWFDGDGMLSGVAFRGISPEDGRIIPEFANQFILTDLYLSKKTTAVSSPIMPSITTLVNPLSTLFQIILSVLRTVFLVLLSHLPGSQQAIKRISVANTSILYYDGRALASCKSGPPIRIQLPSLDTVGWFNGIQAEGEQEDFKSQLDARQFGGHDALSFLKEWTTGHPKVDKRTGEMLLYHNTFLSPFVHYSVIQSSLSKNSNTPKLKLINEPVAGVSGARMLHDFGASHTQTIIMDLPLSLDSPNVLRNREVVSYDPSKPSRFGVFPRHKPHEDTTAEGKVVSVNLLACRMASATLIYSAGNIAPPNVSSNTKHIELEKTPLTRKTRYRSPRKTMCHYEKGSVLESSASTPNAPNYPSTHASEDNDQCRLCYYEFDLSATEKNQVSHEWALSAIPFEFPSVRPDCEIQRARYVYGCTTSSSCIGVALGKAVKIDVIAKIDSKTLIQNGKDMNITPVTGCVDERTVGEILDEDDKEGSIQCFRLPRNHFAHEPRFIPRSSSDEEDDGYLLFYVFDKSQLTMSGECPQSAVSELWILNAESMRNVVAKITLPQRVPYGLHGIWFSHSDIEKQRDVKTFRSLEQLRTRKDEGVFGRKQWWCSWIRWRDVLEKAVG

SEQ ID NO: 126 - protein, 707 aa, Polychaeton citri
  REGION 1..707: note = misc_feature - Polychaeton citri CBS 116435, KAF2724708.1
MAVVGQKRKRAVSGNIQPTPQAKHPYLSGNFAPIHQTLPLTPCMYSGTIPEELADGEYVRNGSNPVSNEDLGRDAHWFDGDGMLAGVSFRKDKNGRIHPEFVNQFVLTDLYLNTISSPRLRVPILPSIATLVNPVASFFLVTLRILRTLILVLLSHLPGSKQSIKKISVANTNILFHDGRALATCESGPPIRVQLPSLETVGWYDGSCAEGEPERLSPEKEATFGQDGGLVSFMREWTTAHPKVDPETKEMLMFHSCFAPPYVQYSIVPQTSDGVSEQALTLRKEKVLNSPVPGITSAKMMHDFGASLTHTVIMDLPLSMDPINQFKGKAPVTYDSEKPSRFGVFPRRSPQDVKWFETDASCIFHTANTWNNTNMKGEVTAVNMLACRLTSATVVFAAGNIAPPVPPKKTIASAKKRMPFFSKYNVKEEPAIFDRAPLLESPSEEKEALIQLSDDHSPWAMDDLFDEEQCRLYFYQFDLRSGQIKHQWALSAVPFEFPSVRPNLEMQEARYIYGCSTTTTEFGSALGKATKIDALVKVDAASLIQSGQARPPKSVTGVLDSRSVLQILDHDDVNDPIRIFHMPEGWYAQEPRFVPSSTNKSEDDGYLLFYAFDECQLGDNGEVPLDSSPIRAKSELWIIDAMDMKTIVGRVHLPQRVPYGLHGIWFTSEQIAEQRPVDSIRSATEALSKKDEGAWMVVRDWVEKLLG

SEQ ID NO: 127 - protein, 708 aa, Aureobasidium subglaciale
  REGION 1..708: note = misc_feature - Aureobasidium subglaciale EXF-2481, XP_013345212.1
MPLFAQKTGLPQHKHAYLSGNFAPISQTMPLTKCTYEGVLPSELAGGEYVRNGGNPVSNEDLGRDAHWFDGDGMLSGVSFTKQPDGSVQPEFVNQYILTDIFLSTVASPRLRSPILPSIATLVNPLASLITIVLRILRTIVLVILSFLPGSTRAIKKISVANTSILYHDGRALATCESGPPMRIALPGLETVGWYDGNRAEGEPDAHDKHDPSFSGNGLLGFMREWTTAHPKVDPTTKEMMLFHSTFFPPYVQYSIIPPTSARSETNKGIVQRPVPGITGAKMMHDFGVSLQHTIIMDLPLSLDPLNLARNKPVIDYDATKPARFGVFPRHHPDQVKYFETTACCIFHTANAWDDFDASGQVETVHLLTCRLTSACLVFSAGNIAAPRPTKETVKAVKKNMPFFSKYNASEQCLYDGAPTLESPLLAREPLIQITSDDEAIPVPSDETDGQVEEIPDEDDQCRLYHYAFSLKSSRITAQYSLSAISFEFPSVHPEFDMQEARYVYGCSTTVSSFNAALGRSVKIDALAKIDAKALIEKGDRLMQLGKLEPVTGCVDTRTVQEVIDSALDNDPIQVFRMPAGWFAQEPRFVPRKAAPGQTLAEDDGYLLTYAFDESQLCADGEVPCDADTANRAKSELWIIDAKDMRSVVGRVALPQRVPYGLHGVWFPEEDILGQKAVDTVRSISEAKSNKNEGIWMNIRASIEGMLS

SEQ ID NO: 128 - protein, 709 aa, Ceraceosorus bombacis
  REGION 1..709: note = misc_feature - Ceraceosorus bombacis, CEH13466.1
MTSGLLNQGERRDPHPYLSGNYAPVRTELPLTACRYEGRVPDEFRDGQYCRNGGNPLSGEDEMRDAHWFDGDGMLTGVLFRRMPDGSVRPEFINRYVLTDVLLATSPRSTRPLLPSIATFSSPYIPLLSVLLGVLRTLALCLLTWLPGSGCSAVEEQERRLKRISVANTSVWFHDGKAFAGCESGPPLRVLLPGMETAGWWSGQDVADRDVDDSEVETKHRKSHGWGKSGVAGLFAEMTTAHPHVDPKTGELLLFHMSFFAPFLRVSIIPPKKGSAEGDSRSRDQRQGAQAPLLGVEVPGIAQPKQMHDAGWSSRHGIMLDLPLSLDPRNMLRGRPMLHYDSAGPTRFGIFPRREPEKVVWFEEAQACLIFHTANAWEEQSEDGRESGAVSLLACRLNSATLVYSAGNTIPPPHALPPGGRAEQCRLHYWRFPDIEQDRQPALTLPRRPSHSFALSAVPFEFPTMNEACAMSEASFVYGASMRSGSFDAGLGRQSAKIDCLAKIDAAKLVRRGLEQGIGTEVAGFCVDDRTVQDILDEQGAQFECAHGSSSDQSSASAAIRIFALPPNHFAQEATFVPRRGAMQEDDGWLVFFVFDESQLDDDGRVLHDATSELWILEAKTMRDVICRVKLPQRVPYGLHGHFFNAQEIASQEPLNDEQIRTWILASQGVVGPGMGTGGKPAVDLYAGPLAFQRRVTAAMRSTLESWLL

SEQ ID NO: 129 - protein, 709 aa, Zasmidium cellare
  REGION 1..709: note = misc_feature - Zasmidium cellare ATCC 36951, XP_033671248.1
MPLAGQKRKRTSSENILPTPQAKHPYLSGNFAPIQQTLPLTKCTFMGRIPEELADGEYVRNGSNPVSNEDLGRDAHWFDGDGMLSGVSFTKDKATGEVQPDFVNQFVLTDLYLNTVSSPRLRVPILPSIATLVNPVASFIYVTLRILRTILLVILSHLPGSKHPIKKISVANTHVLYHDGRALATCESGPPIRIQLPGLETVGWYDGASAEGEPAKETEDKESVLGQDGGLISFMREWTTAHPKVDPNTKEMIMFHSSFAPPFVQYSIIPQTAQVESGVQNGDASGKILNAAVPGVKSAKMMHDFGVSREHTIIMDLPLSLDPINSLKGLPPVSYDSSRPSRFGVFPRRKPKQVKWFETDASCIFHTANSWDEADSSGQTATVNMLACRLTSATLIFAAGNIAPPVEKREKAVAIAKKRMPFFSKYDQDAEQTYFEQSTALESPCEEKEPLLHIAKADREDDYDDLFNEDQCRLYYYQFDLRSGSINYQWALSSIPFEFPSVRPDLEMSAARYIYGCSTTTTNFGSALGKATKIDALVKIDATALITRGRTTPPRSVTGVVDSRSMAQVLESNDPHDPIQVFQMPEGWYAQEPRFVPASSNASEDDGYLLFYAFDESQLDSNGDVPTDSSPLRAKSELWIVDAQGMKDIVARVKLPQRVPYGLHGTWFSAEQIKEQRPVDCIRTTAKAMEGKGEGWWMSTRDQLERMLG

SEQ ID NO: 130 - protein, 714 aa, Rachicladosporium sp.
  REGION 1..714: note = misc_feature - Rachicladosporium sp. CCFEE 5018, OQO21131.1
MVVAGQKRKRGGSDNILPTPQPRHPYLTGNFAPIDKTIPLTPCTYTGTIPEELADGEYVRNGSNPVSNSDLGRDAHWFDGDGMLAGVLFRKDETTGSIQPEFVNQYILTDVYLSSIGSKRLKVPILPSIATLVNPLSSFFWVILRILRTILLVILSHLPGSKQKIKKISVANTNIVYHDGRALALCESGPPLRIQLPGLETVGWYDGATAEGEPVDAQSTEKERVLGEGSGLISFMREWTTAHPKLDPKSKEMLMFHASFAPPYVQYSIVPQSKTTDQAGAPMQKVLNAAVPGVRGARMMHDFGVSSSHTIIMDLPLSLDPLLQLQGKPPVSYDSSKPSRFGVFPRREPEKATWFETDACCIFHTANSWDVVDASGNTTAVNMLACRLTSATMIFATGNIAPPAPPKKTSTDALPKKRMSFFSKYNLDTEASVYERHGRVESTEEEKEALLHVTSTEISSIENDDDGEDHHFDEDQCRLYYYHFDLSTSQIIHQWALASIPFEFPSVHPDLEMSEAKYVYGCSTSTTSFGSALGKATKIDILVKVDVTTLIARGRANPPRSVSGVVDPRPMADVITSNDPADPIRVFQMPDGWFAQEPRFVPAATKRSEDDGFLLFYAFDEAQLTLTGDAPLDEGEARARSDLWVVDARDMRTVMAQVRLPQRVPYGLHGGWFTGEQVRGQRSVERVRSVEKVLEEKQGGVWMGIRDSIEKFLA

SEQ ID NO: 131 - protein, 721 aa, Cercospora berteroae
  REGION 1..721: note = misc_feature - Cercospora berteroae, PPJ52044.1
MFQLTRAGGGSRFITNTRAGQKRKRSPSTNIQPTPQAKHPYLSGNFAPIQQTLPLTPCTYTGSIPEELAEGEYIRNGSNPVSNDDLGRDSHWFDGDGMLSGVSFTKDEHTGDVTPEFVNQFVLTDLYLNTRGSDKLRVPILPSIATLVSPVASFIWVTFRILRTVLLVLLSHLPGSKQKIKKISVANTNVIYHDGRALATCESGPPMRIQLPGLETVGWYNGVSAEGEPIDEKDEQDKDKAFGQDGGLISFMREWTTAHPKVDPVSKEMLLYHASFAPPYVQYSVIPQASGTSAAAGTRLVNVGIPKVSGAKMMHDFGVSSQHTIIMDLPLTLDPINQLKGLPTVHYDASKSSRFGVFPRHKPEETQWFETDGCCIFHTANSWDNVEPHGHTSSVNMLACRLTSATLIYAAGNMAPPPLKPLSSSVVKKKKRMPFFSSYDTGNGQTNFDPDQPLDEKEPLLRVGERYTSIYEDERDPLAEDQCRLYYYSFDLNSTNITHQWALSVVPFEFPSVHPGREMSEARYVYGCSTTTTDFGAALGKATKIDAIVKMDAKKLIQRGRANPPKSVTGVVDIRTMAQVLETSDPEDPIQVFVMPPGWFAQEPRFVPCKDSTSEDDGYLLFYAFDEGQLTPDGDVPADTCEGRAKSELWIVDAKNMQKVVGRVQLPQRVPYGLHGSWFSAEMIKNQREVASTRSTATALDRNERGGLWMKVRGTLEGWLG

SEQ ID NO: 132 - protein, 723 aa, Baudoinia panamericana
  REGION 1..723: note = misc_feature - Baudoinia panamericana UAMH 10762, XP_007674358.1
MLEGAPTLAAGQKRKRTSSSHTIQPTPQAKHPYLTGNFAPIQQTLPLTPCTYTGDIPDELADGEYVRNGGNPVSNEDLGRDAHWFDGDGMLSGVSFRRDQKAGRIVPEFVNQYVLTDLYLSTLSSPRLRVPILPSIATLVNPVASVIYVTFRIFRTLFLVLLSFLPGSKQKIKKISVANTHIVYHDGRALATCESGPPMRVQLPGLQTVGWYNGAWAEGERKAEEAQAKKQQEVERVLGQDGGMIAFMREWTTAHPKLDPVTKEMIMFHSSFARPYVQYSIIPQEAREPVDEKRSLQPQSKMLNAAVPGIYSAKMMHDFGVSTGHTVIMDLPLALDPMNQMKGKPPMSYDSSQPARFGVFPRRHPEQVRWFETDACCIFHTANSWDTVDAAGYTTSVNMLACRLTSATLVFATGNVAPPAARKPKTVAMAKRMSFFAKYDDPMNASVYERSALLESPLEEQEVPNRRGEAAADSEEYDSVWDENECRLYYYSFCLKTGQIEHQWALSTIAFEFPSVRTDLEMQAARYVYGCGSSVTSFGSALGKATKIDLLIKVDVQTLIARGLQRPPRSVTGSVDTRSIEEILASNDDNDPIRAFQLPEGWFAQEPRFVPASTNSSEDDGWLVFYAFDESQLTVDGEVPSDNSPLRARSELWIVNARDVRTLVARVHLPQRVPYGLHGSWFTADQIREQRAIENVRSTAKALEKREVGWWMSCRDVIERMLG

SEQ ID NO: 133 - protein, 728 aa, Acidomyces sp.
  REGION 1..728: note = misc_feature - Acidomyces sp. 'richmondensis', KXL45605.1
MLSGLPAPLAGSKRKRASSFGEINPTPQARHPYLSGNFAPIHQTVHLTPCSYTGRIPRELAGGEYVRNGSNPVSNEDLGRDAHWFDGDGMLSGVSFAKDATTGEIRPHFANQFVLTDLYLSAISSPRLRVPILPSIATLVNPLVSFIYVTLRILRTVALVILSFLPGSAQSISRISVANTHVVYHDGRALALCESGPPMRVQLPNLETVGWYNGSWAEGEREKVRQTEKPVVKEAVLGQGSGIISFMREWTTAHPKIDPVTKEMMLFHASFMPPYVQYSILPQQSGPLSESAASFAQKEKMLNEAVPGVTGGRMMHDFGVSLTHTVIMDLPLSLDPMNQLKGLPPLVYHSGKPSRFGIFPRRSPQSVRWFETDACCIFHTANTWDNLDSVGRTTDVNMIACRLTSATVVFAAGNIAPPVPPKKTVAVSEQKKRRMPFFSKYDHDLELTIFERSTFLESPVEEKEAFIRMDHSATHDNEEQSCSHLWDEEQCRLYYYKFNMTTNQITYQWALAAVPCDFPSVNPQKEMQNARYIYGCSTSSKSFGAALGKATKIDVLVKVDAHRLIERGMRRPPKSVTGCVDTRTLEQVLTSTDADDPIRAFKMPEGWFAQEPRFVPASSGSQEDDGFLLFYAFDESQLNEAGEVPLDTSPLRAKSELWIIDAKGMKDIVCRVHLPQRVPYGLHGTWFSEEQIQSQRPVHSVRTTSKARQGKEQNAWMRIRDCIETLLG

SEQ ID NO: 134 - protein, 733 aa, Teratosphaeria nubilosa
  REGION 1..733: note = misc_feature - Teratosphaeria nubilosa, KAF2773889.1
MLEATPFVVATQASTPQASTKRNRTSSSHELRPTPQPKHPYLSGNFAPIHQTLPLTPCSYSGRIPEELAGGEYVRNGSSPVSNEDLGRDAHWFDGDGMLAGVLFRRDTEDGDVKPEFVNQYVLTDLYLSTISSPHLKVPILPSIATLVNPLASVIYVTLRIIRTLMLVLLSFLPGSKQSIKKISVANTHILYHDGRALATCESGPPIRIQLPELETVGWYNGAWAEGEKSDEAAEEGQPDQKEKVLGEDGGLISFMREWTTAHPKVDPITKEMLMFHASFVPPYVQYSVIPRQEKGSEKMTAGPSRQKMLNAAVPGVRGGRMMHDFGVSLKNTIIMDLPLSLDPIMQLRGLPPVTYDSGKPSRFGVFPRHRPDLVHWFETDACCIFHTANSWDTVDAAGRTTNANMLACRLTSATVIFAAGNIAPPVERQKVIAVAEQKKRRMSFFSKYDHESEKSIFERSGLVESPSEEKEAFIKLSIDAAQAIDDKGPWDEEQCRLYYYDFDLTTGQIAHQWALAAVPLEFPSVAPELEMQNARYVYGCSTSTTNFGSALGKATKIDVLLKVDAHTLIQCGKRQPPRSVTGTVDTRNMDQVLASHDLEDPVRAFRMPEDWFAQEPRFVPAASRRSEDDGYLLFYAFDETQLDGYGDIPSDSSELRANSELWVVDARTMKDVVCRVHLPQRVPYGLHGSWFPEEQISGQRALEHVRTTAKATEGRENGLWMAIREWSERLLA

SEQ ID NO: 135 - protein, 735 aa, Pseudocercospora fuligena
  REGION 1..735: note = misc_feature - Pseudocercospora fuligena, KAF7196919.1
MVFQYDSMEGGGGGGGIGSTVMSTLGKRKRSPSTNIERTPQPKHPYLSGNFAPIQQTLPLTPCTYTGRIPEELADGEYVRNGSNPVSNSDLGRDAHWFDGDGMLSGVSFSKDEETGEIKPEFVNQFILTDLYLNAKSNGRLGVPILPSIATLVNPLASFIFVTLRIIRTILLVILSHLPGSRQKIKKISVANTNVVYHDGRAMALCESGPPMRIQLPGLETVGWFNGAVAEGEPAKESEEKVAVLGENSGLISFMREWTTAHPKVDPQTKEMLMFHSSFAPPYVQYSIIPQTARAEEEGAAPPTRKMLNAGVPGIKSAKMMHDFGVSSEHTVIMDLPLSLDPLNQLKGLPPVAYNPDRPSRFGVFPRRDPKQTKWFETNACCIFHTANTWDERALDGSTTSVSMLACRLTSATLIYAAGNMAPPVPPQAKALEESEAKRRKMPFFSKYDPEVDASFVESYDEKEPLLRVPSLEEHLLKDPFAEEQCRLYYYNFDLASGIISHQWALSAVPFEFPSVRPDLEMSQARYVYGCSTTTTDFGSALGKATKIDALVKIDATKLIQRGRTLPPRSITGVVDGRTMSQVLISRDPRDPIKVFRMPEGWFAQEPRFVPRERSAHERQLNDDPLDEDDGYLLFYAFDETQLLASGEVPDDTSKAKRAKSELWIVDARNMRDVIARVHLPQRVPYGLHGSWFSAEQIAEQRPVERMRSMDEVMKERHDSGLWMKVRERIEMWLG

SEQ ID NO: 136 - protein, 736 aa, Quercus suber
  REGION 1..736: note = misc_feature - Quercus suber, Cork oak; UPI000CE26881
MSVYAGQKRKRSRVENIPFTPQARHPYLSGNFAPIQQTLPLTKCKFTGSIPHELGDGQYVRNGSNPVANGDLGRDAHWFDGDGMLAGVLFRRNSETGTIEPEFVNQYILTDLYLSTLASPALKVPILPSIAVLVNPIASIFYVMLRIVRTLLLVLLSHLPGSRQKIKRISVANTNVVYHDGRALATCESGPPMRIQLPGLETVGWYDGANAEGETLRGAVDIKQEKVLGQDGGLISFMREWTTAHPKVDPKTKEMLMFHSSFAPPYVQYSIIPQTPRESLKQTTNTTQRKLLNAAVPGITSAKMMHDFGVSLQHTVIMDLPLSLDPMNQLKGKPPVTYDSSKPSRFGVFPRHTPSEVTWFETDASCIFHTANSWDEDGEDGKTKNVNMLACRLTSASLVFAAGNIAPPVEKVRRPPVGRSWKKQRMPFFSKYSAQDDVVDGSMLDLECPIEEKESLLHITEPSAIVDCSEAELLEDQCQLYYYSFSLATKQIQHQWALSTIPFEFPSVHPDLEMQNARYIYGCSTTSTTFGAALGKATKIDALVKVDATTLIARGHARSPRSVTGAVDERSMAQVLASQHDPTDPIKVFQMPPGWYAQEPRFVPATPSPSPALSREDDALSREDDGYLVFYAFDEAQLDPATGDVPDDTHATLRARSELWIVDARDMSTLLARIHLPQRVPYGLHGTWFAKEQIDAQRAVHELRTTRDVCLPLDERGRAGGSMMMMLRRWVEEMLG

SEQ ID NO: 137 - protein, 738 aa, Elsinoe fawcettii
  REGION 1..738: note = misc_feature - Elsinoe fawcettii, KAF4553202.1
MTVAGQKRKRGQASAKHPYLAGNFAPIQQVVPLTPCTYEGRIPDELAGGEYVRNGGNPVSNEDLGRDAHWFDGDGMLSGVAFTKEEDGRIQPEFVNQYVLTDVYLSTISSPHNKTPILPSIATLVNPVASFFWITWRVMRTIFLVVLSFLSGSKQAIKKISVANTSVWYHDGRALAGCESGPPMRINLPGLDTVGWYDGCGAENEPKDEHAQDGKRIGGEGLLGWMREWTTAHPKVDPVTKEMLLFHSSFAPPYVQYSVIPEQRTPSRAEKQDYSPLKKYVNLPLPGVSSAKMMHDFGVSSMHSVIMDLPLSLDPLNMVKGKPVVEYDPTKPSRFGVFPRHSPDQVRWFETKACCIFHTANTWDDMDQQGQVKSVHLLACRLTSGSTVFSAGNIAGPVPRNSMVQEKKKGMPFFSKYDNDFERQVVRPGDEQQSYFDDPSAAYSDDIEAAPLLREPTQRGQHIDEAVPARTPGSEVFELPEEIWEEWTDSEEDQCRLYHYNFSLESGRITSEYALARIPFEFPSVHPSFEMKRARYAFGCSTSCASFGAALGKSAKIDILVKIDAQRLIAEGERQLAAGLLQPVTDCVDKRSLSQILDSNDPNDTIQAFHMPHGWFAQEPRFVPRHNSTSEDDGFLLTYAFNEEQLTAEGEVPSDDDATLRSKSELWIIDAKDMRTVIGKVQLPQRVPYGLHGTWFSEDEVQGQRAVETVRSTAQALSKRDTGGVRMKIRDSVEKVLA

SEQ ID NO: 138 - protein, 740 aa, Sphaerulina musiva
  REGION 1..740: note = misc_feature - Sphaerulina musiva SO2202, XP_016756175.1
MYLHHNDNEDDHDRDTRFTTTSLAGQKRKRACSTNILPTPQAKHPYLSGNFAPIQQTLPLTPCAFTGRIPEELADGEYVRNGSNPVSNEDLGRDSHWFDGDGMLSGVSFTRDEQTGAVQPEFVNQFVLTDLYLNTLGSERLRVPILPSIATLVSPVASFVWVTLRILRTIVLVVLSHLPGSRQKIKKISVANTNVVFHDGRALATCESGPPMRIQLPGLETVGWYNGASAEGEMVDGVEGKEKEKVLGEDGGLLSFMREWTTAHPKVDPVSKEMLLYHASFAPPYVQYSVIPQTPAAGMARSQKLLNVGVPKVSGAKMMHDFGVSSGHTVIMDLPLTLDPINQLKGLPTVHYDSSKPSRFGIFPRHSPELTKWFETDGCCIFHTANSWDSVNSLGHVSSVNMLACRLTSATLIYAAGNMKPPPVKPLVSVTSKKNKRMPFFSKYDPGTDHTNFDPTEPMSEKEPLLRIGERYTSIYEDERDPLAEDQCRLYYYSFDLATTSITHQWALSAVPFEFPSVHPTREMQEARYVYGCSTTTTDFGAALGKATKIDAIVKIDAHSLIARGRASPPKSVTGVVDSRTMAQVLESSDPQDPIQVFRMPPGWYAQEPRFVPAASSHSREEDEEEEEEEEEEDNGYLLFYAFDETQLTAAGDVPPDDDSTRRATSELWIVNAKNMTEVIGRVQLPQRVPYGLHGCWFSAEQIQSQRPVECIRTTVKALEERESRGLWMGVRDLVERWVG

SEQ ID NO: 139 - protein, 744 aa, Filobasidium floriforme
  REGION 1..744: note = misc_feature - Filobasidium floriforme, KAG7562916.1
MTLFPLGKKARRRSSIHPYLAGNFAPVLEEYISHPCEIIEGVVPEELHGSQYVRNGGNPVNPPDEGRNYHWFDGDGMLHGVFFPKATVAGESPSPPLYTNRHVSTPILSLSLMLLRAPLPSISLLISPLTSLHRILGAIVTTFLLAFEAGVGTLSVVNTNVIWWGSGLGSEAEEERSKEGADGVIKKTGRLLGTCESGPPMEIRVPELETVEWDRLEDEQGESLREKYAKWDPRGWRLARLQEDWMTAHPRVDPVNGDLVFYQCTMFEKPYLRYSVIDRHGKHRTWKMGIDIGKAKMMHDFACSRKFTIFLNLPLTLSPFNMLSWPPRPMIHFDRNLPSEFIIMPRRTDASTKPIRFIDPEPSMIFHTANAWDDQCEKSGDAVIDMVACRFKSAKLVYAAGAVAIPAEEVRAGFDDVVRLHYYRFLFESDGAHRISHNFPLSEIPFEFPATHPSKSMSKARFVYGCSMREGGFDERLGGAAKVDVLVKVDVIALSEQGRLRGEGKAKAVDQRTVLQILEDHKNGRHDGIQVFVMPSGWFAQESRFVPRNIEGADEDDGWLLTYVYDEQYIDAFGRASNAPGSGSELWIIDAKTMWKGQSSVVCRLKLPQRVPYGLHGTWIPGAHVDMQRDLEHPITPQPTLVHKIMASRFRHFVDIFTARPRSRSRTLAERMAFWVLFLGSWIALGQAVLDISNDQDMRQGAVAWLSSHKSSLTRYLEPVSGLQLLGGLGIGGVLLVLLGGSIS

SEQ ID NO: 140 - protein, 745 aa, Mixia osmundae
  REGION 1..745: note = misc_feature - Mixia osmundae IAM 14324, XP_014567302.1
MGARDWRSLRKGKAREDEKRSSHPYLIGNFRPVEKEHHLTPCTVVEGSIPRELWGGQYIRNGGNPAFPPKEKTAYHWFDGDGMLHGVHFRISTTADGVHSVEPHYTNRYIQTDVFLASKTRGSTILPSISSLLISPSDSLGTKAALVPMLIRTVAIAYLSSLTRLSVTNTAVYFHNRRLLATCESGPPMRIALPELTTVGWYVHPDENGEGGLGRGELTVGAKEWIGAGRFAGAMLEEWQTGHPKLDPISGEFLTYGYNIFARPYVTYSVIGPHSEHRIFKQPVEIPTAKMMHDWGVSRKHTIIMDLPLTMSPFNIAKGLKPMVYFNRKLKSRFGIMPRYFSGQQSDIRWYETDPCLIFHTANAFDETDEAGAVNAVSLLACRFATAKLVHAAGGRPAPAQEEAIAAALPDGDIVRLTYYRFDLSTSKNTITHEFALSNIPYEFPVANPAAVMRETQYLYGCTMRRGRFDEAMRAAKIDSFVKFDSRALVAKGKAQGFTRSGIVDTRSVMDVIEQQSTYGPDHVPTDPVRIYALPAHHYAQEPAFVARQDASAEDDGYLLFYVYDERQLDSLGSASDDTKGELWIIDSMKIGRSATHEDALVARISVPQRVPYGLHCNFISQAQIASQRREEWQAPKPVLTAAQIRAARTTEAIDVLLGEQDIGQRRDSLARLELAWLLYLSIAGWLVLRSAPPHSSHVLLVAMIRSARDVCMTLLAARLFFIFQPRFAVLRHIFSAKHRTPASP

SEQ ID NO: 141 - protein, 754 aa, Violaceomyces palustris
  REGION 1..754: note = misc_feature - Violaceomyces palustris, PWN51250.1
MAWLKRAQAPRQLQPYQPSPTNSEKVQDECLKRARAPPTSLTARPPLGRRESTSKRSPTHPYLSGNFAPVSTEYPLTDCLYQGTIPSELAGCQYVRNGGNPMANSDADRDAHWFDADGMLAGVLFRRLPDGSIQPSFLNRYILTDLILSTPDQLRRPILPSIATLVNPGTSLLWLLVQILRSFLLIMLSWLPGLGLRSEHKVRRISVANTSIYWHDGKAMAGCESGPPIRIILPGLETGDWWTGEDQDGGGEADRVEDNQHLHEVDKGWGSRIPVVGMLREFTTAHPKVDPVTGELLLYHMTFVPPHLRISVFPGSSVAKDGAERRVKAIKGVAVPGLSQPKMMHDFAASSSQTVVLDLPLSLDPVNVLKGRPILHYDPTKASRFGVFPRRSPASVKWYESPEACCVYHTANCWDEYQDENEGEMGKRSSRLAAVNMLGCRLNSATLVYSAGNLVPPNHALPPGNAPEKCQLYYWRFSDPPSDSSSAEERDGEATISHEFALSEVPFEFPSMNEAYSMSEARFVYGTSLKDGTFDAGLGKAAKIDCLVKMDVGRLIQKGRRLWSAGQLERGGSVDRRTVEMILEDQRCGITNDAIRIFEMPSGWYAQEATFVARRKDLGEGAEAEEDDGWLLCYVFDENSGLHPTTGDVLPGATSELWIIDAKRMDEVVCRIKLPQRVPYGLHGTLFSEEQIASQRPIEAERVRSWARSINKQDPMRRYGVKERSWTNLVEAFLADPIRVMAWEVKRGLEGLIA

SEQ ID NO: 142 - protein, 755 aa, Testicularia cyperi
  REGION 1..755: note = misc_feature - Testicularia cyperi, PWZ01013.1
MASSTSGPKRLRPGSLKRQGRQLSRGPTLEIPEPFATALCGTSSSASESASSAFPSSSKVKLVPPRPTLTSKKEYIHPYLSGNFAPVTTECPLTDCLWAGQIPNELAGCQYVRNGGNPLANSQLDRDAHWFDADGMLAGVLFRRREDGSLQPSFLNRFILTDLLLSTPEHSQLPYLPSIATLVNPHTSFLRILSQILRSFLLAILTWLPGLGLRSDEKLRRISVANTSVFWHDGKAMAGCESGPPMRIMLPGLETAGWYTGKDDDNDPSSKGFGDSSLPMLGMLREFTTAHPRIDPQTGELLLYHMCFEPPFLRVSVIPSSTAEKAPVLSMKARANAKVLKGSPVRGLQQPKMMHDFGATSTHTVILDLPLSLDMMNLVKGKPILHYDPSKPSRFGILPRYEPDQVRWFDSPEACCVYHTANSWNDDRKIGAESCVTSAAEEPAAVNMLGCRLNSATLVYSAGNLLPPSHVLPPPDCPEKCQLYYWRFDLESNKISHEFALSDVPFEFPSINEDYSMKEARYVYGTSMRDGLAKIDALVKVDVHTLIRRGKQKWAQGKLERGDSVDTQEILESQRCGDTACEGEEAIQIFEMPRGWYAQETTFVPRDIVNAAEDDGWLICYVFDEGQGLHPSTGEVLPGATSELWIIDAKGMKDVVCRIKLPQRVPYGLHGTLFTEEQIQSQKPIDPSKIRSWASSVNNMDPFSSSSLGSSFVGTIGKKSSGAFPSSREAYNAFLRDPIRIGAWWIKRNIEFLLA

SEQ ID NO: 143 - protein, 761 aa, Friedmanniomyces endolithicus
  REGION 1..761: note = misc_feature - Friedmanniomyces endolithicus, TKA42309.1
MFEPPLPLAGHKRKRASSHLLQETPQPRHPYLTGNFAPIQQTLPLTPCTYTGTIPVELAGGQYVRNGSNPVSNEDLGRDAHWFDGDGMLAGVLFRQDEENGEIQPEFVNRYILTDLYLSTLSSPRLRVPILPSIATLVNPLYSLFYVTLRILRTVLLVILSFLPGSKQKINKISVANTNIVYHDGRALATCESGPPMRIQLPELETVGWYNGAWAQGEEDGDGVLDKEEMRELHGKKLGEDSAFALLGWIREWTTAHPKVDPVTKEMLMFHSSFAPPYVQYSIIPQQQHTPNPNTPAVSYEHTTQPRLEKLLNATVPGVAKAKMMHDFGVSLSHTVIMDLPLSLDPVNQLRGLPPVTYDSSQPSRFGVFPRRQPDDVRWFETDASCIFHTANTWDTSEVDEAGNTTTTEVNMLACRLTSATLIYASGNIAAPVERKPKVVLAETKKKRRMPFFSKYDDAESTVYERAALLESPDEDEEKEPFVHINPGPSPSPFTAPDETLNEDSPSWEEDQCRLYYYAFDLSSPATTNRIAHQWALTTIPFEFPSVRPDREMSAARYIYGCSTSSTSFGSALGKATKIDVLVKIDALGLVERGRASPPRSVGGSVDTRSMAAILASAAAEDPVKGFQMPEGWFAQEPRFVPKRDGVGEDDGWLLFYAFDEGQLLPSGDVPGEDGGVGGEGRAKSELWVLSTRDMKTVVARVRLPQRVPYGLHGSWFDGEMIRGQRGVEGTPRTVGSVRGGETGGGGVWGASRRWVERMLG

SEQ ID NO: 144 - protein, 769 aa, Pseudozyma flocculosa
  REGION 1..769: note = misc_feature - Pseudozyma flocculosa PF-1, XP_007876729.1
MASPGKPPRHAKPPLIHLPTPPGLSFSSTTEPPVIPDGDAIPRRPVLERSKSSKKCSVHPYLSGNYAPVTLEYPLTDCLVSGQIPAELAGCQYVRNGGNPMANTETDRDAHWFDADGMLAGVLFRTLPDGTIQPSFLNRYILTDLVLSTPERSRYPFLPSIATLVNPRQSLISVLLAILRTVLLAFLTWLPGLGLKRDQKLKRIGAANTSIYWHDGKAMAGCESGPPMRIMLPGLETAGWWTGEEDPEEQTQDAASTAGPVSKEEKGRKGKGWGSGPPILGMLREFTTAHPRVDPISGELLLYHMCFEPPFLRVTVIPATQKGSQPNTRKLLGVPVRGLSQPKMMHDFGATRTRTVILDLPLSLDMMNMVKGRPILDYDPTKPSRFGIFPRHQPDEVRWYESPEACCVFHTANSWDGEDDSVNMLACRLNSATLVYSAGNLLPPSHVLPPANCPEKCQLYYWRFQSEPEEVAEVAASRSRSSKGKAEGNRVNVISHEFALSDIPFEFPSINEERGLQEARYIYGTSLKDGTFDAGLAKAAKIDCLVKVDAERLIRRGKQLWSQGKLGRGESVDTRTVVEVLAQQRERAPGSPPDTIQVFEMPRGWYAQEATLVPRARKAGQAELAEDDGWLMFYAFDERTGLHPTSGEVLPEATSELWIIDAKSMTDVVCRIKLPQRVPYGLHGSLFNEEQIASQRPVPASQIRTWADSINNVDPFSLSSTSKSFAGAIDLKLSKDSTRSSAEMYDAFLQRPIHVSAWWLKRTIESWIA

SEQ ID NO: 145 - protein, 781 aa, Moesziomyces antarcticus
  REGION 1..781: note = misc_feature - Moesziomyces antarcticus, XP_014658716.1
MVVSQHAMHRSGSLKRPTGQKPTAPTISIPQPPALSEGPTYPPSDYLADSAEAGPSRPKAIPRRPVLSRRRTSKKDYVHPYLSGNFAPVTTECPLTDCLYEGTIPEEFAGSQYVRNGGNPLANSELDRDAHWFDADGMLAGVLFRRTPDGSIQPCFLNRFILTDLLLSTPEHSRLPYLPSIATLVNPHVGLLWLMLGILRTFVLTVLTWLPGLGLRSDQKLRRISVANTSVFWHDGKAMAGCESGPPMRIMLPGLETAGWYTGEDEEPSTVDKAAEKKRGKASEGFGGGPPIVSMLREFTTAHPKVDPHTQELLLYHMCFEPPYLRVSVIAPAEVVAKKMPPQAKTIKGQAVRGLKQPKMMHDFGATPTHTVIIDVPLSLDLMNLVRGRPILHYDPAQPIRFGVMPRYAPEQVRWFESAEACCVYHTANSWNDDGKFGASRTELVEPTEAGRSEPTGINMLGCRLNSATLVYSAGNLLPPSHVLPPPNCPEKCQLYYWRFDLEDVDAERISHEFALSEVPFEFPTINEEYAMRDARYVYGTSMRDGTFDAGLGKAAKIDALVKIDAQTLIRRGKALWSQGRLGPGDSVDTRSVEEILAAQRDGSGAPDDAIRIFEMPRGWYAQETTFVPRRSGAGPHEEDDGWLVCYVFDEAAGLHPSTGEVLPGATSELWIIDAREMKTVVARVKLPQRVPYGLHGTLFTEDQIAQQNPIDPAKIRSWAQSVTHADPFSASQLGSTLCTAPGKAASSKFSSAKHVYAAFLADPLHIGAWWIKRKIELLIA

SEQ ID NO: 146 - protein, 791 aa, Sporisorium scitamineum
  REGION 1..791: note = misc_feature - Sporisorium scitamineum, CDU24508.1
MVAADRDNRRRSSSMRKSNSQQHYVPIISISSPPPPAEEYAYPPSSSTTNLLIDEKSQAEAGPSHAITSTAQPSHIPRRPVLSRRKTSKKEYVHPYLSGNFAPVTTECPLTDCLYEGTIPQEFAGSQYVRNGGNPLANSDLDRDAHWFDADGMLAGVLFRRIVDGTIQPCFLNRFILTDLLLSTPDHSRLPYLPSIATLVNPHTSIFWLLCEIIRTFVLTILTWLPGLGLTSDQKLKRISVANTSVFWHDGKAMAGCESGPPMRIMLPGLKTVGWYTGEDEEEGEDEKHRDADDRKTKSKGFGGGPPIISMLREFTTAHPKIDPKTQELLLYHMCFEAPYLRVSVIPPARSKRMHLPAHATTIKGKPVRGLKQPKMMHDFGATPTQTIILDVPLSLDMLNLVRGKPILHYDPSQPSRFGVFPRYAPEQVRWFESPEACCVYHTANSWDDDGKFASSHVHLSDANTTRGVNMLGCRLNSATLVYSAGNLLPPSHVLPPPNCSEKCQLYYWRFDLEHEDANRISHEFALSDVPFEFPAINEEYLMQQARYVYGTSMHDGTFDAGLGKAAKIDALVKVDAETLIRKGKAMWSQGRLKAGESVDTRTVEEVLAAQRDGSASAEDAIKIFEMPRGWYAQETTFVPRRSGAQQEEDDGWLVCYVFDEAMGLHPSTGEVLPGATSELWIIDAKDMKDVVCRVKLPQRVPYGLHGTLFTEEQIASQRPIDPSQVRSWASSVNLADPFSSSSLGSTVYTASGKAAIGKFKDGKEVYAAFVKDPNRIGAWWIKRKIELFIA

SEQ ID NO: 147 - protein, 330 aa, Ilumatobacter sp.
  REGION 1..330: note = misc_feature - Ilumatobacter sp., TVR27654.1
MTATALDQPPVRERPSRGFGAVVAITALIAAVGALTTDVPTSISIGVVVVVVALLGLPHGAVDHLVEAAISGHDPTTRTSRLRFHVWYLGAMAGYGLVWLVAPTVALAGFLLVSIHHFGQSDLAHLRLPGASQLAVQWSRGLFVIGLPLVAHLAVVSPVIGRLGGGDPASWSWLADRWALWSAAIVVQHVVVGAVVARRCDDRAAVRREAVTVAVLGALFVSVDPLIGFAVYFGLWHSLAHLRVLAEVLGGASSADGRRTLPLRRFVALVAPRAVLSALGLIGLVAAMYATGRPDLVLPAVFVLVSVLTVPHLVVVERLWRHRTSPSRSR

SEQ ID NO: 148 - protein, 320 aa, unidentified
  REGION 1..320: note = Balneolaceae bacterium, MBL6916485.1
MATHAKTNNEIIQLQVFVGLAIIGLNFFLYEWFEAIRYYALAASVLIIGMPHGALDHKIYFKALHKEETIKTQIVFYAGYLLVVAVYAWLWYVQPFIGFVLFMLFTLYHFGQSDTERLPLNGFLKQVFILARGLSIAGLILFGSDPFYTSRIVESVTGISLISIVYNFTTTEDLRLFFALMYPSVFLFGSLVSKQVPLKSWLSLDALVVPALFSLVDPIFAFALYFGLWHGYNHTKTMLDFLSSKNEQIGFGWFYKETALFSAISYIGLVLIYFLVEAFGDEALLVAILFTLISVITLPHMLVVEQMYRAFDKTDSDQST

SEQ ID NO: 149 - protein, 275 aa, unidentified [sequence omitted]

SEQ ID NO: 150 - protein, 579 aa, Mucor circinelloides
  REGION 1..579: note = misc_feature - McCrtI; A0A168PH23; Mucor circinelloides
MSKKHIVIIGAGVGGTATAARLAREGFKVTVVEKNDFGGGRCSLIHHQGHRFDQGPSLYLMPKYFEDAFADLDERIQDHLELLRCDNNYKVHFDDGESIQLSSDLTRMKAELDRVEGPLGFGRFLDFMKETHIHYESGTLIALKKNFESIWDLIRIKYAPEIFRLHLFGKIYDRASKYFKTKKMRMAFTFQTMYMGMSPYDAPAVYSLLQYTEFAEGIWYPRGGFNMVVQKLEAIAKQKYDAEFIYNAPVAKINTDDATKQVTGVTLENGHIIDADAVVCNADLVYAYHNLLPPCRWTQNTLASKKLTSSSISFYWSMSTKVPQLDVHNIFLAEAYQESFDEIFKDFGLPSEASFYVNVPSRIDPSAAPDGKDSVIVLVPIGHMKSKTGDASTENYPAMVDKARKMVLAVIERRLGMSNFADLIEHEQVNDPAVWQSKFNLWRGSILGLSHDVLQVLWFRPSTKDSTGRYDNLFFVGASTHPGTGVPIVLAGSKLTSDQVVKSFGKTPKPRKIEMENTQAPLEEPDAESTFPVWFWLRAAFWVMFMFFYFFPQSNGQTPASFINNLLPEVFRVHNSNVI

SEQ ID NO: 151 - protein, 560 aa, Sporidiobolus pararoseus
  REGION 1..560: note = misc_feature - Sp.CrtI; A0A0K0QVD9; Sporidiobolus pararoseus
MSNTEKAYGNKNGHASGRPKAIIVGAGIGGCASAARLTQGGFDVTVLEKNDFAGGRCSTFEGAPNYRFDQGPSLYLLPKLIAESFKDLGTSLDQEGIKLVKCEPNYRIVFPDKESVEMSTDLSRMKKEVERWEGQDGFEGFLGFMKEAHVHYELSYEHVLHRNYTTLASIIRPTLLTQLRLLISVTSTVYTRASRYFKTERMRRAFTFASMYLGMSPFDALGAYNLLQYTEHCEGILYPLGGFSIVPKTLAKIAERNGANFRFNTPVERVIVENGQAKGVITESGEELRADVVLVNADLVWAMGHLYKETSYSKRLEEKPVSCSSISFYWSMNRKIPQLNSHTIFLAEEYRESFDSIFTEHKIPHEPSFYVNVPSRHDASAAPEGKDSVIVLVPVGHISPALPTASDWNRIVDETREKIIGEIERRLDIKDLKKDIINEVVNTPITWSEKFNLHRGSILGLSHSFFNVLSFRPKTRHPSVKNAYFVGASAHPGTGVPIVLAGARLATTQILQDYNLPIPASWEVSSAELATASAKTDSNGLILLALLIALICALVTYARN

SEQ ID NO: 152 - protein, 595 aa, Neurospora crassa
  REGION 1..595: note = misc_feature - Nc.CrtI; P21334; Neurospora crassa
MAETQRPRSAIIVGAGAGGIAVAARLAKAGVDVTVLEKNDFTGGRCSLIHTKAGYRFDQGPSLLLLPGLFRETFEDLGTTLEQEDVELLQCFPNYNIWFSDGKRFSPTTDNATMKVEIEKWEGPDGFRRYLSWLAEGHQHYETSLRHVLHRNFKSILELADPRLVVTLLMALHPFESIWHRAGRYFKTDRMQRVFTFATMYMGMSPFDAPATYSLLQYSELAEGIWYPRGGFHKVLDALVKIGERMGVKYRLNTGVSQVLTDGGKNGKKPKATGVQLENGEVLNADLVVVNADLVYTYNNLLPKEIGGIKKYANKLNNRKASCSSISFYWSLSGMAKELETHNIFLAEEYKESFDAIFERQALPDDPSFYIHVPSRVDPSAAPPDRDAVIALVPVGHLLQNGQPELDWPTLVSKARAGVLATIQARTGLSLSPLITEEIVNTPYTWETKFNLSKGAILGLAHDFFNVLAFRPRTKAQGMDNAYFVGASTHPGTGVPIVLAGAKITAEQILEETFPKNTKVPWTTNEERNSERMRKEMDEKITEEGIIMRSNSSKPGRRGSDAFEGAMEVVNLLSQRAFPLLVALMGVLYFLLFVR

SEQ ID NO: 153 - protein, 614 aa, Mucor circinelloides
  REGION 1..614: note = misc_feature - Mc.CrtYB_Q9UUQ6, Mucor circinelloides
MLLTYMEVHLYYTLPVLGVLSWLSRPYYTATDALKFKFLTLVAFTTASAWDNYIVYHKAWSYCPTCVTAVIGYVPLEEYMFFIIMTLLTVAFTNLVMRWHLHSFFIRPETPVMQSVLVRLVPITALLITAYKAWHLAVPGKPLFYGSCILWYACPVLALLWFGAGEYMMRRPLAVLVSIALPTLFLCWVDVVAIGAGTWDISLATSTGKFVVPHLPVEEFMFFALINTVLVFGTCAIDRTMAILHLFKNKSPYQRPYQHSKSFLHQILEMTWAFCLPDQVLHSDTFHDLSVSWDILRKASKSFYTASAVFPGDVRQELGVLYAFCRATDDLCDNEQVPVQTRKEQLILTHQFVSDLFGQKTSAPTAIDWDFYNDQLPASCISAFKSFTRLRHVLEAGAIKELLDGYKWDLERRSIRDQEDLRYYSACVASSVGEMCTRIILAHADKPASRQQTQWIIQRAREMGLVLQYTNIARDIVTDSEELGRCYLPQDWLTEKEVALIQGGLAREIGEERLLSLSHRLIYQADELMVVANKGIDKLPSHCQGGVRAACNVYASIGTKLKSYKHHYPSRAHVGNSKRVEIALLSVYNLYTAPIATSSTTHCRQGKMRNLNTI

SEQ ID NO: 154 - protein, 614 aa, Mucor circinelloides
  REGION 1..614: note = misc_feature - Mc.CrtYB_Q9UUQ6_Y27R, Mucor circinelloides
MLLTYMEVHLYYTLPVLGVLSWLSRPRYTATDALKFKFLTLVAFTTASAWDNYIVYHKAWSYCPTCVTAVIGYVPLEEYMFFIIMTLLTVAFTNLVMRWHLHSFFIRPETPVMQSVLVRLVPITALLITAYKAWHLAVPGKPLFYGSCILWYACPVLALLWFGAGEYMMRRPLAVLVSIALPTLFLCWVDVVAIGAGTWDISLATSTGKFVVPHLPVEEFMFFALINTVLVFGTCAIDRTMAILHLFKNKSPYQRPYQHSKSFLHQILEMTWAFCLPDQVLHSDTFHDLSVSWDILRKASKSFYTASAVFPGDVRQELGVLYAFCRATDDLCDNEQVPVQTRKEQLILTHQFVSDLFGQKTSAPTAIDWDFYNDQLPASCISAFKSFTRLRHVLEAGAIKELLDGYKWDLERRSIRDQEDLRYYSACVASSVGEMCTRIILAHADKPASRQQTQWIIQRAREMGLVLQYTNIARDIVTDSEELGRCYLPQDWLTEKEVALIQGGLAREIGEERLLSLSHRLIYQADELMVVANKGIDKLPSHCQGGVRAACNVYASIGTKLKSYKHHYPSRAHVGNSKRVEIALLSVYNLYTAPIATSSTTHCRQGKMRNLNTI

SEQ ID NO: 155 - protein, 602 aa, Phycomyces blakesleeanus
  REGION 1..602: note = misc_feature - Pb.CrtYB; Q9P854; Phycomyces blakesleeanus
MLTYMEVHLYFTLPVLALLAFLYKPFFTTKDRFKYIFLCTVAFATASPWDNYIVYHKAWSYCPECVTAVIGYVPLEEYMFFIIMTLITVTFTSLTMRWTLPSFFIRPETPVFQSVCVRYIPIVGFLTIAAKAWASSIPDSHPFYGACILWYVCPVLALLWIGSGEYMLRRWKAVLFSIAVPTIFLCWVDQYAIARGTWDISRRTSTGIMVLPSLPLEEFLFFLLIDTVLVFASCATDRAHAIVHIYITPMNHNKVSTWYMDFFYLCWAFLQTDQALSGETLSDLDATWRILREASASFYTASSVFSFEARQDLGVLYGFCRATDDLADNNDVSVPDRKKQLELVRGFVRQMFDSKHGHPDIDWTQYSGSIPDSFIAAFRSFTRLRDVLEIKAVEELLDGYTFDLEQREVKNEDDLVYYSACVASSVGEMCTRVLMASEPGGNRTMLKWTVERARDMGLALQLTNIARDIVTDSKQLGRSYVPRDWLTSQESALLKAGKARELGDERLRQIALKMVYTADDLNLMASRAIDYLPPSSRCGVRAACNVYTAIGVSLHKANGYPDRAHLTKLERMKVTFRCVYGFRKGHQGVQGDRGKSQAFTVI

SEQ ID NO: 156 - protein, 602 aa, Neurospora crassa
  REGION 1..602: note = misc_feature - Nc.CrtYB; P37295; Neurospora crassa
MYDYAFVHLKFTVPAAVLLTAIAYPILNRIHLIQTGFLVVVAFTAALPWDAYLIKHKVWSYPPEAIVGPRLLGIPFEELFFFVIQTYITALVYILFNKPVLHALHLNNQQNPPAWMRVVKVTGQVVLVALSVWGWNAAQVHQETSYLGLILVWACPFLLAIWTLAGRFILSLPWYATVLPMFLPTFYLWAVDEFALHRGTWSIGSGTKLDFCLFGKLDIEEATFFLVTNMLIVGGMAAFDQYLAVIYAFPTLFPKVNRYPTTHMLLQSRLINTSRYDLERIEGLREAVERLRLKSRSFYLANSLFSGRLRIDLILLYSFCRLADDLVDDAKSRREVLSWTAKLNHFLDLHYKDADATEDPKKKAERIDAYIKTAFPPCAYQALHLLPTHILPPKPLYDLIKGFEMDSQFTFHGTSDSTDLQYPIADDKDLENYAIYVAGTVGELCIALIIYHCLPDMSDTQKRELETAACRMGIALQYVNIARDIVVDARIGRVYLPTTWLKKEGLTHKMVLENPEGPEVIERMRRRLLENAFELYGGARPEMQRIPSEARGPMIGAVENYMAIGRVLRERKEGTVFVRMEGRATVPKRRRLSTLLRALYEQ

SEQ ID NO: 157 - protein, 352 aa, Saccharomyces cerevisiae
  REGION 1..352: note = misc_feature - Farnesyl pyrophosphate synthase P08524
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAINDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 158 - protein, 544 aa, Aspergillus kawachii
  REGION 1..544: note = misc_feature - Ak.CrtI_G7XI79, Aspergillus kawachii
MAKKSAIVVGAGVGGISTAARLAREGFKVTVLEKHAFVGGRCSIIERDGYRFDQGPSLLLMREIFSRTFQDLGTSLSSEGVELLKCEPNYCIWFPDNDIVELSSDITRLKEEVIRHEGITGLSKLLDFLKETGQYYNLTLDYIMNRDFPHFLSLLQPNVLRALVAMRPWNTMAGIVEQYFTSTKMKQAWTFASMYMGMSPYQAPGTYSLLQYSETVDGIWYPRGGFQKVLQALADVGRRLGVDYRLRAPVDSVLLAPDQRSAKGVRLNSGEEIHADLVVINADLVYAYNNLLPESGYGRSLQKRHTSCSSISFFWAFDTTFPQLKSHNIFLAEKYRESFDSIFVDHSIPDEPSFYLNVPSRIDPTAAPPGKDAVVVLVPVGSLREPGTNDKKHWERIIPDIRDLVIKTIEERTGLQNLRSKLLHETYETPHTWKDKFNLDRGAILGLSHSFFNMLSFRPSLKHPTIDGLYFAGASTHPGTGVPVCLAGSKLVCERILRDTRLEQRMLFATAYIWVTFTLVVVLATACVTLLYPSCLDSFLCLVQ

SEQ ID NO: 159 - protein, 578 aa, Apophysomyces ossiformis
  REGION 1..578: note = misc_feature - Ao.CrtI_A0A8H7BN47, Apophysomyces ossiformis
MSQKHIIIIGAGVGGVSTAAKLSREGFQVTVLEKNNFVGGRCSFIHHDGHRFDQGPSLYLMPKLFEEAFADVDERITDHLELLRCQNNYKIYFDDGDAIQLSSDLSHMKAEMERIEGQDGFGNFLKFMGETHVHYTRGVVLALKQNFESFWDMVRLKHAPEIVRLHLFSKIYSRASRYFRTKKMRMAFTFQTMYMGMSPFDAPAVYSLLQYTEFAEGIWYPRGGFNMVAQKLEAIAAQKYGAKFIYEAPVAKINTDAKRVTGVTLENGEVIEADAVVCNADLVYAYHNLLPPCQWTQNHLAKKKLTSSSISFYWSLKQVVPQLDVHNIFLAEAYRESFDEIFQDYSLPSEISFYVNIPSRIDPTAAPPNKDTMVVLVPVGHMRGLETSSEEKYYAAMVNRARRMVLEVLAQRLGLENFHDLIEHEIVNDPTVWQSKFNLWRGSVLGLSHNILQVLWFRPSTKDSTGRFDNLFFVGASTHPGTGVPIVLAGSKLTADQVCRHFGKVPTPSKLANQQKQRIYESEKQPVSWTWWYPLIFLFVLAFAFFVSFPENQSSETAASFINTCLPSMFRVESASLQ

SEQ ID NO: 160 - protein, 544 aa, Aspergillus oryzae
  REGION 1..544: note = misc_feature - Ao.CrtI_I7ZZQ8, Aspergillus oryzae
MSSIPIGILSRPESRQLIAPGAGAGGIATAARLAQEGFHVKVVEQHGFIGGRCSIISKDGYRFDQGPSLLLMREVFEETFQDLGTSLEQENVRLVKCEPNYCVWFPDKDIIELSTNLTRLKAQIQHHEGPDGFSRFCAFLNEAGTHYNLSLAHVLRKNFPGFLSLLRWDVLRSLISMHPWTSTYSRAARYFYSEKMRRVWTFGSMYLGMSPYRAPGTYSLLQYIETVDGIWYPAGGFQRVLRALGDIGMRSGVEYILNSPVKSVLLDDSNHVAKGVLLEDGEELYADLVVINADLVYAYNELLPKTRRSHDLKKRPVSCSSISFFWSFDEKLPHLRAHNIFLAEKYRESFDAIFEDHRIPDEPSFYVNVPSKIDPTAAPPGKEAVVVLVPVGHLTSEKGGQLEEEKWDTLVSQTREIVLDTIEARTGLQDLRSRLVHEMVETPLSWEERFNLDRGAILGLSHSFFNVLSFRPQIKHPDIERLYFVGASTHPGTGVPVCLAGSKLVTQQIVEDWNMGIRQKPRSGFILTLVMALLTLVVSFLWRH

SEQ ID NO: 161 - protein, 542 aa, Aspergillus ruber
  REGION 1..542: note = misc_feature - Ar.CrtI_A0A017RZD5, Aspergillus ruber
MTPKAVVVGAGAGGVATAARLAKQGFQVTVIEKNGFIGGRCSLITHNGYRFDQGPSLLLMPEVFHDTFNQLGTSLEEEGIKLLKCEPNYRLWFTDNDSFDLSTDIASLKPQIEKYEGKEGFQNFLQFMQESGRHHDLSLIHVLRRRFPSLLSMLRPGFLLSVFKMHPFESIHGRLSRYFRSEKMRRIFTFASMYLGMNPYEAPGTYSLLQYTELADGIWYPAGGFQKVLESIATIGKSFGVEYRLNSPVSSILLSGDSKSATGVVLSSGERLDADIVVINADLVYAYQNLLPASKEACRLSNRPASCSSISFFWSFDRVVPELKAHNIFLAEKYEESFDAIFKYHKLPDEPSFYVNVPSRIDRTAAPDEKDAVVVLVPTGHLLENTESITEADWITIVSKARETAINTIEARTGVRGLRDILVHESIETPVSWKEKFNLDRGAILGLSHSFFNVLSFRPKNKHPDIARLYFVGASTHPGTGVPVCLAGSKLVVDQVVEDWNADKKSSLAGICLVIVSMLIVMVSLMPFVTFCHHALHTVISS

SEQ ID NO: 162 - protein, 610 aa, Absidia repens
  REGION 1..610: note = misc_feature - Ar.CrtI_A0A1X2IZQ9, Absidia repens
MTPPKHIIVIGAGAGGTASAARLARQGFRVTVVEKNDFNGGRCSLIYHNGYRFDQGPSLYLMPKLFEDTFADLDEKLDDHIDLLRCSNNYKVHFDDGDKIHLTSDLTLMKTEMERVEGEHGFSRFLDFMSETHVHYENGTLLAIKRNFATIWDMIKIKYAPEIFRLHLFDKIYNRASLYFKTKKMRMAFTFQTMYMGMSPYDAPAVYSLLQYTEFAEGIWYPRGGFNTVVQRLESIARDKYGAKFIYNAPVARINTKDNQQQVTGVTLENGTVIEADAVVCNADLVYAYHHLLPPCPWTTSTLAKKKLTSSSISFYWSLSQTVPELDVHNIFLAENYKESFDEIFQDHSLPSEPSFYVNLPSRIDPSAAPPNKDAMIVLVPIGHMRSESGKKADQQEMDDYEVMVERARTMVLTVLERRLGINNFRHMIDHEIVNHPGIWQEKFNLWRGSVLGLSHDVLQVLWFRPSTKDSTGRYNNLFFVGASTHPGTGVPIVLAGSKLTSDQVCQHFNQIPMPRLQQQKMTETTDGGVISNNKKKVFEYKPEMEYNNGFILSNTLWYWCASLFLLTLTFFVAFPTNHTNHTAASYINSSMPSIFRVGKYECTMEDMCR

SEQ ID NO: 163 - protein, 504 aa, Aurantimonas sp.
  REGION 1..504: note = misc_feature - As.CrtI_Q1YGX8, Aurantimonas sp. SI85-9A1 s
MSATMNGAGAGERTAIVIGSGFGGLALAIRLQASGIQTTLLEKRDKPGGRAYVYEDQGFIFDAGPTVITDPSALEELFTAAGRTMADYVELLPVKPFYRLCWEDGYSFDYANDQDELDRQIHAKNPADIEGYRQFLAYSREVFREGYEKLGTVPFLSFRDMVKAGPQLAKLQAWRSVYSKVAQYIEDEQLRQAFSFHSLLVGGNPFATSSIYALIHALEREWGVWFPRGGTGALVRGLVRLFEDMGGRVELSAEAAQIEVVGDRAKAVVLKDGRRFEADRIASNADIVHTYGTLMAGTRRGEKEAKRLKARRFSMSLFVIYFGLKRHRPELQHHTVCFGPRYRPLIDEIFKGGELPGDFSLYLHSPCATDPSLAPEGAGSYYVLAPVPHLGNTDIDWEIEGPKYRDRILDYLEERYIPDLRADLVTSRIFTPFDFRDELNAHVGSAFSLDPVLTQSAWFRPHNRDADIPNLYFVGAGTHPGAGVPGVVGSAKATAGLMIADSRA

SEQ ID NO: 164 - protein, 528 aa, unidentified
  REGION 1..528: note = Bl.CrtI_Q9KK84, Brevibacterium linens
MTERIGVIGGGAAGLATAALLAREGHSVDLFEQNSTLGGRIGSIEEDGYRFDTGPSWYLMPEVYEHFFNLLGTTAANELDLRTLDPAYTVFSPASQARREQSVTIPHGTAEIIDTFERIERGSGATLAKYLDSAKATKDLAIDYFLYNPFTSPKSILRAELLTALPALARLLTTSLERHASRSFHHPVLRQILQYPAIFLGTDPRKAPALYHLMSALDLSDGVLYPMGGFRTIVDALTRLVEESGVRVRTDSTVTHISTVSESGRRRSANGIAWKDQDGREHFHDCDIVVSAADLHHTETQLLGEGERSYPESWWQKVTSGPGAVLVFLGVEGEVPQLPHHSLFFTEDWSTNFDDIFGSQQKISSPASAYVCKPSHTDPNVAPFDCENLFILIPVPADAGLGAGGDDGQGDPRIERAADQAIDQIAEWADIPDLRDRIRFRRTLGPTDFSSNYNSWLGGMLGPAHTLRQSAMFRQQNASKKVDGLYYAGATTAPGVGVPMCLISAELVLKHIRGDSSPGPLPVTDTRE

SEQ ID NO: 165 - protein, 512 aa, Bradyrhizobium sp.
  REGION 1..512: note = misc_feature - Bo.CrtI_Q7BP90, Bradyrhizobium sp. (strain ORS 278)
MLDVSPLKAAVPAALGDRPHAVVIGSGFGGLAAAVRLGARGYRVTVFEQLDAPGGRAYVHRQDGFTLDAGPTIVTAPFLFEELWQLCGRRMSDDVTLKPISPFYRIRFDDGTIFDCSADPAAMRAEVARISPRDVDGYERFMMTSEAIFRKGFEELGDVPFLSLFDMAKIAPAMIKLQSFRSVYGLVAQHVLDERLRVILSFHPLLIGGNPFTTTSIYCLIAYLERRWGVHFAMGGTSALVSGLVGLIEGQGGEVRCSAPVAEITTTNGAATGVRLTSGETIAADVVVSNGCAAWTYKNLLPSVRRRRWTDARIDRARYSMSLFVWYFGTKRRYEDVEHHTILLGPRYRELLDDIFRRKVLADDFSLYLHRPTATDPSLAPNGCDAFYVLSPVPHQESGIDWTVAAEPFRKRIETALSQTLLPDLASQIVTSRLLTPDDFEQRLSSYRGAAFGLEPVLMQSAWFRPHNKSEEVERLYLVGAGTHPGAGLPGVLSSARVLDDLVPAPHELERR

SEQ ID NO: 166 - protein, 506 aa, Bradyrhizobium sp.
  REGION 1..506: note = misc_feature - Bo.CrtI_Q9KIX2, Bradyrhizobium sp. ORS278
MSQEMQNAKTAVVIGSGFGGLSLAIRLQSAGIATTLVEKRDKFGGRAYVYEQDGFTFDAGPTVITDPTCLQELFALSGRKLENYVELMPVSPFYQLRWEDGATFDYVNDQAELERQIAAFCPADVDGYRRFRSYSERLLEEGYVKLGHVPFPDFRSMVRVAPQLVALQSYRSVYSKVSQYVSDEHLRQAFSFHSLLVGGNPFATSSIYALIHALERRWGVWFPRGGTGALINKGLVQLFKDLGGEVTLSTSVSRIETANGRVSAVVAEDGRRFAADIVASNADVVHTYRDLLKDEPLARPTAQSLMRKRFSMSLFVIYFGLRREHPELKHHIILFGRRYRELINEIFKGPALPEDFSLYLHAPSVTDPSLAPQGCSTYYVLSPVPHLAAAPIDWSVEGPRYRDRILDYLEARILPGLKSDLATCRIFTPQDFNTELNAHLGSAFSLEPILTQSAYFRAHNADDKIKGLYLVGAGTHPGAGIPGVVGSAKATARVILEDQRELAEAQ

SEQ ID NO: 167 - protein, 493 aa, Brevundimonas sp.
  REGION 1..493: note = misc_feature - Bs.CrtI_Q4W8B6, Brevundimonas sp. SD212
MRAAVIGSGFGGLSLAIRLQTAGIQTTVFEARDLPGGRAYVYKDKGYTFDAGPTVITDPSALEELFEGAGRKLSDYVELLPVAPFYRLCWEDGDVFDYVNGQDELDRQIVARNPADKEGYRRFLAYSQDLLKEGYLKLGAVPFLDFASMVKAAPELMRLQAWRSVYDKVAGYIQDEHLRQAFSFHSLLVGGNPFATSSIYALIHALERRWGVWFPRGGTGALIQAMVRLFQDLGGEIRLNSPVERITLANGRADGVVVGGQALAFDMVASNADVVHTYQRLLGQEPRGRKEGARLASKRHSMSLFVIYFGLKRVHPEVRHHTVLFGPRYRELIGEIFKGPDLPQDFSLYLHAPTRTDPSLAPEGCDAFYVLAPVPHLASADIDWAVEGPRYRDRVLAYLEQHYIPGLTAHLDTCRIFTPVDFRDQLNAHQGSAFSLEPILTQSAYFRVHNRDDQIPNLYFVGAGTHPGAGVPGVVGSAKATAGLMIEDAGRTA

SEQ ID NO: 168 - protein, 494 aa, Brevundimonas vesicularis
  REGION 1..494: note = misc_feature - Bv.CrtI_Q0H2D1, Brevundimonas vesicularis DC263
MMRAAVIGSGFGGLSLAIRLQSAGIQTTVFEARDLEGGRAYVFKDKGYTFDAGPTVITDPSALEELFEASGRKLSDYVELLPVAPFYRLCWEDGDVFDYVNDQDELDRQIVARNPADKEGYRKFLAYSQDLLKEGYLKLGAVPFLDFASMVKAAPELMRLQAWRSVYDKVASYIQDEHLRQAFSFHSLLVGGNPFATSSIYALIHALERRWGVWFPRGGTGALIQAMVRLLKDLGGEVRLNSPVERITMANGRATGVVVNGEALAFDMVASNADVVHTYQRLLGQEPRGQKEGAKLASRRHSMSLFVIYFGLKRVHPEVRHHTVLFGPRYRELIAEIFKGPDLPDDFSLYLHAPTRTDPSLAPEGCDAFYVLAPVPHLASADIDWAVEGPRYRDRILAYLEERYIPGLTADLDTCRIFTPVDFRDQLNAHQGSAFSLEPILTQSAWFRVHNRDDQIPNLYFVGAGTHPGAGVPGVVGSAKATAALMIEDARQPA

SEQ ID NO: 169 - protein, 582 aa, Choanephora cucurbitarum
  REGION 1..582: note = misc_feature - Cc.CrtI_A0A1C7N0F9, Choanephora cucurbitarum
MSEQKKHIIVIGAGVGGTATAARLAREGFRVTVVEKNDFSGGRCSFIYHDGHRFDQGPSLYLMPKLFEDAFADLDERIEDHLDLLRCDNNYKVHFDDGDAVQLSSDLTKMKAELERIEGPLGFGKFLDFMKETHVHYEQGVFIAIKRNFETIWDLIRLQYVPEIFRLHLFGKIYDRASKYFQTKKMRMAFTFQTMYMGMSPYDAPAVYSLLQYTEFAEGIWYPRGGFNMVVQKLEAIASEKYGAQFKYQSPVAKINTADKDKRVTGVTLESGEVIEADAVVCNADLVYAYHHLLPPCNWTKKTLASKKLTSSSISFYWSMSTKVPQLDVHNIFLAEAYKESFDEIFNDFSLPSEASFYVNVPSRIDSTAAPPNKDSIIVLVPIGHMKSKTGNATEENYSELVNRARKMVLEIIERRLGVSNFSSLIEHEEVNDPSVWQSKFNLWRGSILGLSHDVFQVLWFRPSTKDSTNRYDNLFFVGASTHPGTGVPIVLAGSKLTSDQVCQSFGQTPLPRKLQDNRKKYAPEKPSQNGSHWIYYCLACYFVTFLFFYFFPRDENTTPASFINQLLPNVFQAQNSNDIRI

SEQ ID NO: 170 - protein, 621 aa, Cercospora nicotianae
  REGION 1..621: note = misc_feature - Cn.CrtI_P48537, Cercospora nicotianae
MPSTSKRPTAIVIGSGVGGVSTAARLARAGFHVTVLEKNNFTGGRCSLIHHEGYRFDQGPSLLLLPGLFHRTFAELGTSLEQEGVKLLKCEPNYMIHFSDGEKFTLSSDLSVMKTEVEKWEGKEGYTRYLEFLKESHGHYELSVREVLLRNFEGLTAMLRPEFLRHLLQLHPFESIWTRAGKYFWTERLRRVFTFGSMYMGMSPFDAPGTYSLLQYTELAEGIWYPVGGFHRVVEALVKIGEREGVDFRMETAVKKILLSEDGGVAKGVELEDGRRLEADVVVNNSDLVYAYEKLLPIKTPYAESLKGRPGSCSSISFYWALDRQVPELEAHNIFLADEYRESFDSIFKKHLIPDEPSFYVNVPSRVDSTAAPEGKDSVVVLVPVGHLLEEDRHASQAHQLSASRNGHISSASPPDQPGLTPTEKQDWPAMISLARKTILSTIQSRTNVDLTPLIIHESTNSPLSWKQTFNLDRGAILGLSHSFFNVLCFRPTTRARKPGAFDAQLLKFGVLGRAAEVIIDAFRGRGKDIKGLYMVGASAHPGTGVPICLAGGALVAEQICGDYGVDIPWKEEERKGRDVGGKRKLDVLESPMWLDSWEQWVSVLIYLLVGIFAWLWMKFR

SEQ ID NO: 171 - protein, 485 aa, unidentified
  REGION 1..485: note = Cp.CrtI_Q4FPE5, Candidatus Pelagibacter ubique HTCC1062
MKSIVIGSGFGGIAAALRLRAKNHKVTLIEKHPDLGGRARVFKKNGFTFDAGPTVITAPYLIEELFTLFNKKSQDYIKLTPLKTWYRFIYEDGGVFDYSGNEDQMKKQIEKINKEDVKGYEQLVKFTKKIFDKGFTELADVPFDKPLVMMKQLPSLLKLKSYKSVYSLVSSYIKNEKLRRMLSMHPLLVGGNPFTTTSIYGLILYLEKKWGIHYSMGGTGNIINGLEKLMIEQEIEVIKGHEVTKIISDNGKISGVKLDNQKEMLSNNVICNADPPAVYEKLLSLNKTSPLFKWKQNRMQYSMGLFVYYFGTKKTYPDVEHHTIKFGDKYKEHLSDIFDNKKLNDDISYYLHRPSATDKSMAPEGNDCFYVLVPVPNNQSKIDWKIEGEKMKNLVIDKMEKALLPNLRENIIEDFYLTPDYFENELNTKFGSGFSIQPKFTQSAYFRFHNKSEIYDGLYFVGAGTHPGAGVPGVLSSAKVLDKIL

SEQ ID NO: 172 - protein, 513 aa, Rubrivivax gelatinosus
  REGION 1..513: note = misc_feature - Cs.CrtI_Q840T3, Rubrivivax gelatinosus S1
MLMKSDAAAPRAAGKQDPQAPVALVVGSGFGGMAAAVRLAAKGYRVTVLEKLDAPGGRAYVHRREGHVFDAGPTIVTVPYLFDELWALAGRKFSDDIELKSLDPFYRIRFDDGDHFDYSGDPARMRAEVRRISPSDAEGFERFMREADQCYELGFRTLGDKAFDTVGDLIKAAPLIIRLRGWRSLHQMVSSHLKHPKLRIAMSLQSLLIGGNPFSVTSMYALVNALERQWGVHWAMGGTGELIRGLVDVFEGMGGTMRLKAEVKRIEVDNGVATGVTLADGERLPADIVVCNGDTGYLYKNLVDARWRKHWTDARIERGHYSMGLFVWYFGTDRRYEDVPHHMMVLGPRYRELLDDIFRKKKLAGDFSIYLHRPTATDPSMAPAGCDTFYALMPVPHLGSGTDWTTQAEPYRQSVQEALERTVLPGLGQHLRVSFCTTPLDFQHRLLSYKGAGFGLEPLLLQSAYFRPHNRSEDVKNLFMVGASTHPGAGVPGVIMSAKALESVLPDPATFRR

SEQ ID NO: 173 - protein, 529 aa, Dinothrombium tinctorium
  REGION 1..529: note = misc_feature - Dt.CrtI_A0A443RCG3, Dinothrombium tinctorium
MNQKAVVIGAGVGGTAVAARLAKQGFEVTVYEKNDFGGGRCSLINKNGHRFDQGPSLYLMPKIFEETFRDLGEDINDHLELLKCESNYKVHFHDGNSIQLTCDLPKMCAEIERFEGEKETTIENFLSFLRECHIHYDHSVRLALKTNFPTLSDLLRPKYLPEVFKMHLYDTVYNRASKYFTSDYMKKAFTFQTMYIGMSPYDAPGAYNLLQATEFLEGIWYPKGGFYQVVKALENIATKKFGAKFVYNCGVKRIEVDADGVATGVTLETGEMIKADVVVCNADLVYAYNKLLPPTNYGDKLGQNCQLTSSSISFYWSMKQKMDQFGVHNIFLAEEYRESFDQIFNDHTLPEDPSFYVNVPNRIDPTAAPPGKDSIIVLVPVGHITDKEKQNFESLVQIARRKVIEIIERCLKIDNFEQYIEDETVNHPLIWSEKFNLWKGSILGLSHNIPQVLYFRPSTRSHLFKRLYFVGASAHPGTGVPVVLCGSKLVADQIAKDRLTSKSDRDSIVKMILVTMFFLLLAVLVSTMF

SEQ ID NO: 174 - protein, 492 aa, Erwinia herbicola
  REGION 1..492: note = misc_feature - Eh.CrtI_Q47845, Erwinia herbicola
MKPTTVIGAGFGGLALAIRLQAAGIPVLLLEQRDKPGGRAYVYEDQGFTFDAGPTVITDPSAIEELFTLAGKQLKDYVELLPVAPFYRLCWESGKVFNYDNDQAQLEAQIQQFNPRDVEGYRQFLDYSRAVFKEGYLKLGTVPFLSFRDMLRAAPQLAKLQAWRTVYSKVASYIEDEHLRQAFSFHSLLVGGNPFATSSIYTLIHALEREWGVWFPRGGTGALVKGMIKLFQDLGGEVVLNAKVSHMETTGDTIEAVHLEDGRRFPTRAVASNADVVHTYRDLLSQHPAAVKQSKKLQTKRMSNSLFVLYFGLNHHHDQLAHHTVCFGPRYRELIHEIFNHDGLADDFSLYLHAPCVTDSSLAPEGCGSYYVLAPVPHLGTANLDWTVEGPRLRDRIFEYLEQHYMPGLRSQLVTQRMFTPFDFRDQLNAYQGSAFSVEPVLTQSAWFRPHNRDKTINNLYLVGAGTHPGAGIPGVIGSAKATAGLMLEDLI

SEQ ID NO: 175 - protein, 526 aa, Erythrobacter longus
  REGION 1..526: note = misc_feature - El.CrtI_O06757, Erythrobacter longus OCH101
MNADQNIATGLNFAPANTGERGINPVIAEKYKGRTACVIGSGFGGLALALRLQSHGIQTTIVEARDKPGGRAYFWEKDGFTFDAGPTVITDPPCLKELWELTGHDISEDVELMKVHPFYRLNWPDGTNFDYSNVDEELNAEIAKLNPDDVIGYQKFLEYSARVHEEGYVKLGTVPFLDFKSMLKAAPALVKERAWRSVYDMVSSYIKDERLREAFSFHTLLVGGSPMKTSAIYALIHKLEKDGGVWWARGGTNRLIAGMVRHFERLGGTMRIGDPVVQVHTQGTKATEVETKSGWKERFDAVCSNADIMHSYKELLGESDRGRKYAKSLARKSYSPSLFVVHFGLEGSWPGIAHHMILFGPRYKELVDDIYKHGVLPQDFSIYLHHPTVTDPSMAPKGMSTFYALVPVAHLGKMPIDWDVEGPKFEKAILDEIGRRLIPDIHDRIVTKFSYAPKDFQADLNAHMGSAFSLETVLWQSAYMRGHNRDDVIDNFYLVGAGTHPGAGIPGVVGSAKATAGLMLEDLSVK

SEQ ID NO: 176 - protein, 492 aa, Erwinia uredovora
  REGION 1..492: note = misc_feature - Eu.CrtI_P21685, Erwinia uredovora
MKPTTVIGAGFGGLALAIRLQAAGIPVLLLEQRDKPGGRAYVYEDQGFTFDAGPTVITDPSAIEELFALAGKQLKEYVELLPVTPFYRLCWESGKVFNYDNDQTRLEAQIQQFNPRDVEGYRQFLDYSRAVFKEGYLKLGTVPFLSFRDMLRAAPQLAKLQAWRSVYSKVASYIEDEHLRQAFSFHSLLVGGNPFATSSIYTLIHALEREWGVWFPRGGTGALVQGMIKLFQDLGGEVVLNARVSHMETTGNKIEAVHLEDGRRFLTQAVASNADVVHTYRDLLSQHPAAVKQSNKLQTKRMSNSLFVLYFGLNHHHDQLAHHTVCFGPRYRELIDEIFNHDGLAEDFSLYLHAPCVTDSSLAPEGCGSYYVLAPVPHLGTANLDWTVEGPKLRDRIFAYLEQHYMPGLRSQLVTHRMFTPFDFRDQLNAYHGSAFSVEPVLTQSAWFRPHNRDKTITNLYLVGAGTHPGAGIPGVIGSAKATAGLMLEDLI

SEQ ID NO: 177 - protein, 501 aa, Erwinia uredovora
  REGION 1..501: note = misc_feature - Eu.CrtI_P54978, Erwinia uredovora
MNAHSPAAKTAIVIGAGFGGLALAIRLQSAGIATTLVEARDKPGGRAYVWHDQGHLFDAGPTVITDPDALKELWALTGQDMARDVTLMPVSPFYRLMWPGGKVFDYVNEADQLERQIAQFNPDDLEGYRRFRDYAEEVYQEGYVKLGTVPFLKLGQMLKAAPALMKLEAYKSVHAKVATFIKDPYLRQAFSYHTLLVGGNPFSTSSIYALIHALERRGGVWFAKGGTNQLVAGMVALFERLGGQMLLNAKVARIDTDGPRATGVTLADGRALTADMVASNGDVMHNYRDLLGHTARGQSRAKSLNAKRWSMSLFVLHFGLREAPKDVAHHTILFGPRYKELVNEIFKGPKLAEDFSLYLHSPCTTDPEMAPPGMSTHYVLAPVPHLGRADIDWAVEGPRYADRILASLEERLIPNLRANLTTTRIFTPSDFASELNAHHGSAFSVEPILTQSAWFRPHNRDKTIRNFYLVGAGTHPGAGIPGVVGSAKATAQVMLSDLASA

SEQ ID NO: 178 - protein, 492 aa, Escherichia vulneris
  REGION 1..492: note = misc_feature - Ev.CrtI_P22871, Escherichia vulneris
MKKTVVIGAGFGGLALAIRLQAAGIPTVLLEQRDKPGGRAYVWHDQGFTFDAGPTVITDPTALEALFTLAGRRMEDYVRLLPVKPFYRLCWESGKTLDYANDSAELEAQITQFNPRDVEGYRRFLAYSQAVFQEGYLRLGSVPFLSFRDMLRAGPQLLKLQAWQSVYQSVSRFIEDEHLRQAFSFHSLLVGGNPFTTSSIYTLIHALEREWGVWFPEGGTGALVNGMVKLFTDLGGEIELNARVEELVVADNRVSQVRLADGRIFDTDAVASNADVVNTYKKLLGHHPVGQKRAAALERKSMSNSLFVLYFGLNQPHSQLAHHTICFGPRYRELIDEIFTGSALADDFSLYLHSPCVTDPSLAPPGCASFYVLAPVPHLGNAPLDWAQEGPKLRDRIFDYLEERYMPGLRSQLVTQRIFTPADFHDTLDAHLGSAFSIEPLLTQSAWFRPHNRDSDIANLYLVGAGTHPGAGIPGVVASAKATASLMIEDLQ

SEQ ID NO: 179 - protein, 509 aa, Fulvimarina pelagi
  REGION 1..509: note = misc_feature - Fp.CrtI_Q0G4C1, Fulvimarina pelagi HTCC2506
MNQMPRDLPNKTKTAVVIGAGFGGLALAIRLQAAGIQTTLLEKRDKPGGRAYVYEDQGFTFDAGPTVITDPSALEELFETANAKLSDYVELLPVKPFYRLAWEDGFVFDYADDQEDLDRQIGAKNPKDVEGYRRFLAYSRDVFHEGYEKLGTVPFLNFKDMMRAAPQLVRLEAYRSVYSKVAQFIEDDQLRQAFSFHSLLVGGNPFATSSIYALIHALERKWGVFFPRGGTGALVRGMAKLFTDIGGRIEVNAEVENIAIENGRAKSVTTKGGQTFPADFVASNADVVHTYAKLMGRSERGKKHGNSLKKKRFSMSLFVIYFGLKTHRPDIAHHTVCFGPRYRPLIDEIFKGKELAGDFSLYLHNPCVTDPSLAPEGMGSFYVLSPVPHLGNADIDWAVEGPKYRDRILDYLEELYIPGLKDDLVTSRIFTPADFKTELNAHLGSAFSLDPVLTQSAWFRPHNRDDQIPNLYVVGAGTHPGAGVPGVVGSAKATAGLMIEDAGLACVPA

SEQ ID NO: 180 - protein, 570 aa, Gibberella fujikuroi
  REGION 1..570: note = misc_feature - Gf.CrtI_Q8X0Z0, Gibberella fujikuroi
MSDIKKSVIVIGAGVGGVSTAARLAKAGFKVTILEKNDFTGGRCSLIHNDGHRFDQGPSLLLLPRFFHEIFQDLGTSLTAEGVELLKCEPNYNIWFGDGSSFEMSTDLTKMKKAIEAVEGIDGFERYLGFLQESHRHYEVSVESVLRRNFPSILSLARPEVLFNLFNIHPLESIWTRASKYFWTERLRRVFTFGSMYMGMSPFDAPGTYSLLQYTELAEGILYPRGGFHKVVEALVNVGQRLGVEYRLSTGVKSISIDQATGKANGVVLSDGTHLPSDIVISNADLVYTYNNLLPKTSYADSLSKRETSCSSISFYWSASKIVPELNAHNIFLADEYQESFDSIFKEHLIPSEPSFYVNVPSRIDPSAAPEGKDSIVVLVPVGHLLSDSEGTHRGLSKSGNSGGLETSQDWDKMISLARDTVIATMRARIGVDLAPLIENEIINTPFTWQEKFNLDKGAILGLSHSIMNVLAFRPGTQHSKYKNLYFAGASTHPGTGVPVCIAGSKIVAEQILKDSGFKNNQIPWAQDTTKSPKGGLDKMSDSSLTLFQGFLGALVAILLAYYYLVIAAN

SEQ ID NO: 181 - protein, 560 aa, Gloeobacter violaceus
  REGION 1..560: note = misc_feature - Gv.CrtI_Q7NM99, Gloeobacter violaceus
MLRSVIKRRTRKGAALERKKAIVIGSGVGGLSLGIRLQSLGFDTTIFEKLDGPGGRAYVRRAEGFTFDMGPTVITVPHFIEELFALERDQPALDKPDFPAAVLDENRRITSGVSGGPATSKYVQIIPILPFYRIYFDDGTHFDYDGDPERTREQIRAIAPEDLEGYERFHRDARAIFERGFLELGFTYFRDVPSMLRIAPDLVKLDAVRPLFSFVKRYFKSDKLRQVFSFEPLLVGGNPLAVPAIYAMIHFVEKTWGIHFAMGGTGALVRGFVRKFEELGGTMRYGSEVARIEIEGSGKHRRATAVVPADGSRHPADLVASNGDYVNTYLKLIDRVHRPSHPDIRLKLARQSMSLLVVYFGFRSDGLNLDLRHHNIILGPRYEELLKDIFGRKILAEDFSQYLHIPSITDPSLAPPGHHCAYTLVPVPHNGSGLDWSKLADPFVDRVLRFLDERGYIPGLGERLVYKSFITPDYFEHTLNSRLGNGFGIEPVLWQSAYFRPHNKSEDIANFYLVGANTQPGGGTPAVMMSAKMTAREIARDFDIPFDVVDRPQVRGTVAK

SEQ ID NO: 182 - protein, 589 aa, Hesseltinella vesiculosa
  REGION 1..589: note = misc_feature - Hv.CrtI_A0A1X2GT92, Hesseltinella vesiculosa
MAQKHVIVIGAGAGGVATAARLARQGFKVTVVEKNDINGGRCSLIHHDGFRFDQGPSLYLMPKLFEDCFADLDENVQDHLDLLRCGNNYKVHFDDGDKIHLTSDLTKMKDEMDRVEGDQGFANFLSFMSESHVHYQQGTLLAIKRNFESVWDMIKLKYAPEIFRLHLLDNVYARASRYFKTKKMRMAFTFQTMYMGMSPYDAPAVYSLLQYSEFAEGIWYPRGGFNMVVQKLEAIARDKFGAKFLYESPVSKINTEGRQVTGVTLKDGRVLEADAVVCNADLVYAYHHLLPPCHWTTRTLGSKKLTSSSISFYWSMSRKIDQLDVHNIFLAENYRESFDEIFQDLNLPSEASFYVNVPSRIDPSAAPGDKDAVIVLVPIGHMQHVEGKWSEGFYDKMVARARAMVLQVLERRLGMTDFGALIQNEIVNHPGVWQQKFNLWRGSILGLSHDILQVLWLRPSTKDSTGRFDNLFFVGASTHPGTGVPIVLAGSKLTSDQVCKHFHQQPLPRKKQDAEKALLNYAYLGEDQASFAFGWTWLLAMFIMTLAFFLAFPKDDVAKTTAASYINARLPEVFRVMGNNCSGDVGQAC

SEQ ID NO: 183 - protein, 571 aa, Jimgerdemannia flammicorona
  REGION 1..571: note = misc_feature - Jf.CrtI_A0A433D8F1, Jimgerdemannia flammicorona
MVDPSQKHIIIIGAGVGGTATAARLGKQGFRVTVLEKNSFSGGRCSLIHHNGHRFDQGPSLYLMPKMFEETFDELGEKLTDHIELLKCQTNYKLHFQDGESILLSPDLAHMKTEIERFEGPGEAPFLKYLGFLAESHVHYEHSMTLALKRNFEAWYEMFQPKFLPELFKLHLFSKIYSRAEKYFSSAKMRMAFTFQTMYMGMSPFDAPGTYNLLQYTELAEGIWYPKGGFHKVVSSLETIANKKFGAQFRYDAPVKQINTAADGKTVTGVTLESGEVIHADAVVCNADLVYAYNKLLPPSNYGKRIGKASFTSSSISLYWGMKRKIPELDVHNIFLADAYKESFDEIFKDHTLPSEPSFYINVPSRIDPTAAPENKDTMVVLVPVGHMTDAHPQDYDSLVAKARSLVLGSLLKHTGVDFAPLIDFEIFNDPRTWQAKFNLWQGSILGLSHNILQVLWFRPSTKDGSGRYDNLFFVGASTHPGTGVPIVLCGARLTSDQVGRHFGVSDEKRGVVKAKGKKVDGAGMIGWFLVLLAVLAVVVGMTGTNGLNNVWEGALKCINTSLLSIIRFQL

SEQ ID NO: 184 - protein, 576 aa, Lichtheimia corymbifera
  REGION 1..576: note = misc_feature - Lc.CrtI_A0A068RLK8, Lichtheimia corymbifera JMRC:FSU:9682
MSSTQKHIIVIGAGAGGTATAARLAREGYRVTVVEKNDFNGGRCSLIHHNGHRFDQGPSLYLMPELFKECFRDLDERIEDHLELLRCQNNYKVHFDDGDTIQLSSDLTRMRPEMDRFEGEDGFGRFLQFLKESQVHYDRGTFVAIKRNFASIWDMIKIQYLPDIFRLHLFDMIYSRASKYFKTKKMRMAFTFQTMYMGMSPYDAPAVYSLLQFTEFAEGIWYPRGGFNMVVQKLEMIAKDKGAEFKYSAPVAKINTTERSVTGVTLENGEVIDADAVVCNADLVYAYNRLLPPCSWAQNTLANKKLTSSSISFYWSMKRTIPELDVHNIFLAEAYRESFDEIFKDYTLPSEPSFYVNVPSRIDPSAAPADKDSIIVLVPIGYMRSTEPDQDYPLLVKRARDMVLEVLQRRIKVDMEPLIEHEIVNDPSVWQEKFNLWRGSILGLSHDVSQVLWFRPSTKDSTGRFDNLFFVGASTHPGTGVPIVLAGSKLTATQVCEHFGRKGLKPSKLVEHKRVYEPESGSTLMSNLVTFIISLFVLLFAFFVSFPTSSVYPTPAAFINACLPSTFRVVMAADIE

SEQ ID NO: 185 - protein, 499 aa, unidentified
  REGION 1..499: note = Ma.CrtI_Q9K566, Mycobacterium aurum A+
MRTVGGRTDNVVVVGAGLSGLSAALHLAGRGRRVTVVERGSHPGGRVGRADLSGYRIDTGPTVLTMPDILDETFAAVGESTADRLELIRVDPAYRASFADGSSLHVHSDAATMAAEIERFAGRAEADGYLRLRSWLARLYQLEFDGFIASNFDSPLSLLTPQLAKLAAIGGFRRWEPMVRRFITDERLLRVFTFQALYAGVPPNRALAAYAVIAYMDTVSGVYFPRGGMRALPDAMAAAAADAGVEFRYDATVTELERTGSRVTAVRTDTGERFPADAVVLTTELPDTYRMLGRTPRRPLRLRPAPSAVVAHIGCRATDPAADAAHHTILFGGEWDRTFEQIIDEGRTMSDPSLLVTRPTAGDPTLAPDGRDLLYVLAPAPNTAVSDKDWDADRDAYTAQMLDTVTTRLPHLGADAEVLHVVTPQDWARQGMLAGTPFALAHTFGQTGPFRPGNRVRGIDNAILAGSSTVPGVGIPTAIVSGRLAADRISSNSMMGART

SEQ ID NO: 186 - protein, 548 aa, Microcystis aeruginosa
  REGION 1..548: note = misc_feature - Ma.CrtI_S3IUA6, Microcystis aeruginosa SPC777
MESYDVILIGAGHNGLVCAAYLLKAGYRVLLLEQRSVPGGAATTEAVIPDLAPDFKFNLCAIDHEFIFLGPVIEELELKRHGLEYLFFDPTVFCPHPSGQYFLSYRSLEKTHAAIAQFCPRDADRYLQFIDYWGQLMNAIGPWFNAPPQDLLRIARNYNSSALASVWKAIGSKKKLLDFIRTMITSPEDVLNEWFESEFVKAPLARLAAEIGAPPSQKGITSGMMMMAMRHSPGVARPRGGTGALTEALVKCVVSLGGVILTEQKVKQILIEEGQAIGVRVDSGGEYLAKAGVISNIDARRVFLQLVAPADVDAADPELRERVERRIVNNNETILKIDCALAEAPRFEAYDHKEEYLQGTILIADSVRHVEQAHALTILGQIPDENPSMYLDVPTILDPSMAPQGKHTLWIEFFAPYQIAGAEGTGLNGTGWTEELKNRVADRVLDKLADYAPNLKSSIIARRVESPAELANRLGSYKGNYYHLDMTLDQMIFLRPLPEIANYKTPIKNLYLTGAGTHPGGSISGMPGRNCAQVFLRDQSTFLQRLFN

SEQ ID NO: 187 - protein, 582 aa, Mucor circinatus
  REGION 1..582: note = misc_feature - Mc.CrtI_A0A8H7S8T5, Mucor circinatus
MSSQEKQKHIIVIGAGAGGTACAARLAREGFKVTVVEKNDFNGGRCSLIHHEGYRFDQGPSLYLMPKLFKETFADLDENINAHLNLLRCQNNYKIHFEDGDKIHLTSDLTKMKPEMERVEGTHGFSRFLDFLNETHEHYERGTLIAIKRNFESLWDMIRIQYAPEIFKLHLFDRIYARASKYFKTKKMRMAFTFQTMYMGMSPYDAPAVYSLLQYTEFAEGIWYPEGGFNMVIQKLEKIARDKYGATFRYESPVAKINTQGNQATGVTLESGEVIEADAVVCNADLVYAYHKLLPPCHWTQNTLASKKLTSSSISFYWSMDRKVTELDVHNIFLAEAYQESFDEIFVDNTLPSEPSFYVNVPSRIDPSAAPADKDSIIVLVPIGHMRGADAASTPEDYQDLVNRAREMVLSVLCRRLGLAHFKDWIAHEIVNDPAVWQQKFNLWRGSILGLSHDVLQVLWFRPSTKDSTGRFDNLFFVGASTHPGTGVPIVLAGSKLTSNQVCNHFSKTPKPSQLLLKEEEQKIIYKPESGMTPSYWYSVLSLFILVFAFFVSFPSEQFGHPTAAAYINSCLPAQFRVAVYE

SEQ ID NO: 188 - protein, 578 aa, Mucor circinelloides
  REGION 1..578: note = misc_feature - Mc.CrtI_S2J0C7, Mucor circinelloides f. circinelloides
MSKKHIVIIGAGVGGTATAARLAREGFKVTVVEKNDFGGGRCSLIHHEGHRFDQGPSLYLMPKYFEDAFADLDERIQDHLELLRCDNNYKVHFDDGESIQLSSDLTRMKAELDRVEGPLGFGRFLDFMKETHIHYESGTLIALKKNFESIWDLIRIKYAPEIFRLHLFGKIYDRASKYFKTKKMRMAFTFQTMYMGMSPYDAPAVYSLLQYTEFAEGIWYPRGGFNMVVQKLEMIAKTKYGADFIYNAPVAKINTNDTTKQVTGVTLENGQIIDADAVVCNADLVYAYHNLLPPCRWTQNTLASKKLTSSSISFYWSMSTKVPQLDVHNIFLAEAYQESFDEIFKDFGLPSEASFYVNVPSRIDPSAAPDGKDSIIVLVPIGHMKSKTGDATTENYPAMVNRARKMVLEVIERRLDMSNFADLIEHEQVNDPAVWQSKFNLWRGSILGLSHDVLQVLWFRPSTKDSTGRYDNLFFVGASTHPGTGVPIVLAGSKLTSDQVVRSFGKSPKPRKIEIENKQAPLEQDVAIGFSLGMWLRIAFLVVFMFFYFFPQSNGQTPASFINNLLPDVFRVHNSNVI

SEQ ID NO: 189 - protein, 564 aa, Mortierella isabellina
  REGION 1..564: note = misc_feature - Mi.CrtI_A0A8H7Q0C1, Mortierella isabellina
MASQKHIVIIGAGVGGTATAARLAKQGFRVTVVEKNSFSGGRCSLIYKNGHRFDQGPSLYLMPKIFKEAFADLDEDIEDHIDLLKCENNYKVHFHDGDSIHLTSDLVKMKTEIERYEGEGEKPFLNYLRFLSESHIHYERSLVMALKRNFEAWYEMFQPKYAPEVFNLHLFHKVYTRAAKYFSSKKMRMAFTFQSMYMGMSPFDAPATYNLLQYTEFAEGIWYPRGGFNMVVQRLETIATAKYGAQFIYNAPVAKINVDEKAKLVTGITLENGQVVQADAVVCNADLIYAYNNLLPPCSWSKRIGEAKLTSSSISFYWCMKRKIPELDVHNIFLAQAYQKSFDQIFKEHTLPSEPSFYVNVPSRIDPSAAPEGKDSVIVLLPIGHMLPGRPEDYQKLVAKARTMILTTLEKRLGFDISKEIEHEEYNDPTVWQEKFNLWRGSILGLSHDILQVLWFRPSTKDSTGRFNNLFFVGASTHPGTGVPIVLCGSKLTSDQVTKHFGYDIKNQKKRTIAYAQNDGTTSIWYGAIIMFMLVFAFFYGFPKDKGTAASFINSYLPLSMRAY

SEQ ID NO: 190 - protein, 586 aa, Mucor plumbeus
  REGION 1..586: note = misc_feature - Mp.CrtI_A0A8H7RIY5, Mucor plumbeus
MSKKHIVIIGAGVGGTATAARLAREGFKVTVVEKNDFGGGRCSLIHHNGHRFDQGPSLYLMPKYFEDAFADLDERIEDHLELLRCDNNYKVHFDDGEAIQLSSDLTRMKSELDRVEGPLGFGRFLDFMKETHIHYESGTLIALKKNFESIWDLIRIKYAPEIFRLHLFGKIYGRASKYFKTKKMRMAFTFQTMYMGMSPYDAPAVYSLLQYTEFAEGIWYPRGGFNMVVQKLEAIAKNKYDAEFIYNAPVSKINTNDTTKQVTGVTLENGEIIEADAVICNADLVYAYHNLLPPCRWTQNTLASKKLTSSSISFYWSMSTKVPQLDVHNIFLAEAYQESFDEIFKDFGLPSEASFYVNVPSRIDPTAAPADKDSIIVLVPIGHMKSKTGDATIENYPEMVKKARKMVLEVIERRLGMSNFSNLIEHEQVNDPAIWQSKFNLWRGSILGLSHDVFQVLWFRPSTKDSTGRYNNLFFVGASTHPGTGVPIVLAGSKLTSDQVVKSFGKAPRARKIEIENKQAPLEENGQISSSFSQLWVWLRVIFWVLFLFFYFFPQSNNGQTPASFINNHLPNVFRAKDSYLDVDMI

SEQ ID NO: 191 - protein, 584 aa, Mucor saturninus
  REGION 1..584: note = misc_feature - Ms.CrtI_A0A8H7RGU3, Mucor saturninus
MSDNKKHIVVIGAGIGGVATAARLSREGFRVTVVEKNDFSGGRCSLIYNEGHRFDQGPSLYLMPKLFQDAFADLDERIEDHVDLLRCDNNYKVHFDDGESIQLSSDLTRMKSELDRIEGPEGFGRFLDFLKETHIHYERGVFIAIKRNFESIWDMVRIKFAPEIFRLHLFGKIYDRASKYFLTKKMRMAFTFQTMYMGMSPYDAPAVYSLLQYTEFAEGIWYPRGGFNVVVQKLEEIGSKKYGAKFIYNAPVAKINTEDENKRVTGVTLENGEVIHADAVVCNADLVYAYHHLLPPCSWTTNTLATKKLTSSSISFYWSLKEKVPLLDVHNIFLAEAYQESFDEIFKDYGLPSEASFYVNVPSRIDPSAAPEGKDSVIVLVPIGHMRSLTGNGDEENYPKMVERARAMVLEIIQRRLGMENFADLIEHESHNDPAIWQSKFNLWRGSILGLSHDILQVLWFRPSTKDSTLRYDNLFFVGASTHPGTGVPIVLAGSKLTSDQVTKSFGKAPLPRKIQDEPTYKAEANDASSQLVTYSVYLFIITFLFFVLFPTQSTNHTAASFINSYLPKQFRVTYDNDLLLERY

SEQ ID NO: 192 - protein, 529 aa, Myxococcus xanthus
  REGION 1..529: note = misc_feature - Mx.CrtI_Q02861, Myxococcus xanthus
MASEGGSVRHVIVVGAGPGGLSAAINLAGQGFRVTVVEKDAVPGGRMKGLTLGASGEYAVDTGPSILQLPGVLEQIFRRAARRLEDYVKLLPLDVNTRVHFWDGTHLDTTRHLDRMEAELAKFGPRQASALRQWMEDGREKYGIAYQKFICTSADNLGYYAPWRLAPTLRFKPWQTLYRQLDGFFHDDRVTYALAYPSKYLGLHPTTCSSVFSVIPFLELAFGVWHVEGGFRELSRGMMRCARDLGATFRMGTPVEKVRVDAGRAVGVKLVGGEVLDADAVVVNADLAYAARSLIPAEAREGSRLTDAALERAKYSCSTFMAYYGLDTVYADLPHHLIYLSESARRTDRDALEDRHVDLEDPPFYVCNPGVTDPSGAPAGHSTLYVLVPTPNTGRPVDWVKTEQALRERIPAMLEKVGLKGVREHIREERYFTAETWRDDFNVFRGAVFNLSHTWLQLGPLRPKVKNRDIEGLYFVGGGTHPGSGLLTIMESANIAADYLTREAGKGPLPGWPYVPPLEPESPVQARAG

SEQ ID NO: 193 - protein, 517 aa, Myxococcus xanthus
  REGION 1..517: note = misc_feature - Mx.CrtI_Q1DDW7, Myxococcus xanthus DK 1622d
MSASTQGRRIVVVGAGVGGLAAAARLAHQGFDVQVFEKTQGPGGRCNRLQVDGFTWDLGPTIVLMPEVFEETFRAVGRRIEDYLTLLRCDPNYRVHFRDGSDVTFTSELCAMGRELERVEPGSYARYLAFLAQGRVQYRTSLDHLVGRNYAGLRDYLSPRVLARIFQVRAHRRMYADVSRFFQDERLRAAMTFQTMYLGVSPYASPAVYGLLPFTELGVGIWFPKGGLYAIPQALERLAREEGVRFHYGAPVERILTDGGRTRGVRLEGGEVVEADAVLCNADLPYAYEKLLDPKATTLKRKEKLRYTSSGYMLYLGMKRRYPELLHHNVVFGRDYKGSFDDIFERFRVPEDPSFYVNAPTRTDASLAPEGKDALYVLVPVPHQHPDLDWKVEGPKVRAKFFARMAELGFPSLESDIEVERVFTPDDWAGTFNLARGSAFGLSQNFTQIGPFRPSNQDARVKNLFFVGASTQPGTGLPTVLISARLVTERLMTWAHAQGVSLSPRTAAATPLEGVAA

SEQ ID NO: 194 - protein, 511 aa, Parvularcula bermudensis
  REGION 1..511: note = misc_feature - Pb.CrtI_E0THV8, Parvularcula bermudensis HTCC2503
MTGQKEAGQKEACVIGGGFGGLALACRLQAAGLATTLIEGRDKLGGRAYVYSLEGYTFDAGPTVVTDPSALEEIFAMAGRRLSDYVELLNVDPLYRLHWEDGYSFDYAKDEEHLLSQIRAKNPKDVEGYRKFYRYSEAVFKEGYEKLGAVPFLNFWSMVKAAPQLTTLQAFRSVYGRVADFIEDEQLRQAFSFHTLLVGGDPHKTSSIYALIHALERKWGVWFPRGGTGALIEGLGRLFTDLGGTLRLEDPVTDLKVQNDRITQVTTESGWSHQFDAVASNADIVHTYDNLLGGSKRGPQAAKGLKQKRFSNSLFVTYFGTKKQYPDIAHHSILFGPRYRELIAEIFSGPQLPNDFSLYLHRPTATDPGLAPPGCDAFYVLSPVPNLEKAGIDWSVTGPEYQERILAYLEERYLPGLRDNLAVTHRFTPADFKDKLNAHVGSAFSLEPVLTQSAYFRLHNRDDKIGNLYFVGAGTHPGAGIPGVVGSAKATAGLMIEDMGLSRSNRGPKAA

SEQ ID NO: 195 - protein, 583 aa, Phycomyces blakesleeanus
  REGION 1..583: note = misc_feature - Pb.CrtI_P54982, Phycomyces blakesleeanus
MAPPKHVIIIGAGAGGTATAARLAREGIKVTVVEKNNFGGGRCSLINHNGHRFDQGPSLYLMPKLFEEAFEALDEKIEDHVELLRCHNNYKVHFDDGDKIQLSSDLSRMKPEMERIEGPDGFLRFLDFMKESHTHYEGGVEMAIKQNFETIWKLIRLQYVPALFRLHIFDFVYSRAAKYFKTKKMRMAFTFQSMYMGMSPYDSPAVYNLLQYTEFAEGIWYPKGGFNTVIQKLENIATEKFGARFIYEAPVAKINTDDKGKKVTGVTLQSGEVIEADAVVCNADLVYAYHNLLPPCRWTTNTLAEKKLTSSSISFYWSLKRVVPELDVHNIFLAEAFKESFDEIFTDHKMPSELSFYVNLPSRIDPTAAPPGKDSMIVLVPIGHMKSKTNEAEDYTMIVKRARKMVLEVLERRLGLTNFIDLVEHEEVNDPSIWQKKFNLWRGSILGLSHDVLQVLWFRPSTQDSTGRYKNLFFVGASTHPGTGVPIVLAGSKLTSDQVCDHFGVKVRPSAITSSKRTYAPEDSKSFIWDIIWFLLIALFAATLVLFIAFPQYSEVNQTAASYINNLLPAAFRVPVANLSLTS

SEQ ID NO: 196 - protein, 518 aa, Paenibacillus sp.
  REGION 1..518: note = misc_feature - Pf.CrtI_A0A089KIW6, Paenibacillus sp. FSL P4-0081
MTGPKRTAKVAIIGAGPGGLAAAMLLAAEGYAVDLYEKQDTVGGRSGELKLGSYRFDRGATFLMMPHLLEELFALAGRSVHDYVSLKPLDPLYSLHFGDTVFTPSTDQEQTAGEIERLFPGNGSGYRRFMADEADKLERVIPLLQRPFQSLGDYMKRDVLHALPKLNAADNVYNRLSRYFDDERLRFSFTFQAKYLGMSPWECPGTFTILSYMEHRYGLYHPIGGINRVLQAMSEVIQEHGGHVHTSSGVKRIVVKNGDAAGLLLENGERVEADYVVIGADFGSAMTQLFEPGILKKYTPRKVARKRYSCSTAMLYLGIDGEVELGHHSVHFSADYRRNVEEITKLGVLSEDPSIYVHNPSVIDKTLAPPGKSSLYVLMPVPNLSAEVNWQQTSAEVEARMMERLEQIPQLAGITGRVEESLFFSPLDWQNKLNVYNGATFNLAHNLGQMMHLRPHNTFEEVGGIWLVGGGTHPGSGLPTIFESAKISARLLREHDQALRGSFTVPAGAPGAGAGVPL

SEQ ID NO: 197 - protein, 501 aa, Paracoccus haeundaensis
  REGION 1..501: note = misc_feature - Ph.CrtI_Q24K61, Paracoccus haeundaensis
MNAHSPAAKTAIVIGAGFGGLALAIRLQSAGIATTLVEARDKPGGRAYVWHDQGHVFDAGPTVITDPDALKELWALTGQDMARDVTLMPVSPFYRLMWPGGKVFDYVNEADQLERQIAQFNPDDLEGYRRFRDYAEEVYQEGYVKLGTVPFLKLGQMLKAAPALMKLEAYKSVHAKVATFIKDPYLRQAFSYHTLLVGGNPFSTSSIYALIHALERRGGVWFAKGGTNQLVAGMVALFERLGGQMMLNAKVARIETEGARTTGVTLADGRSLRADMVASNGDVMHNYRDLLGHTARGQSRAKSLDRKRWSMSLFVLHFGLREAPKDIAHHTILFGPRYRELVNEIFKGPKLAEDFSLYLHSPCTTDPDMAPPGMSTHYVLAPVPHLGRAEIDWAVEGPRYADRILASLEERLIPNLRANLTTTRIFTPADFASELNAHHGSAFSVEPILTQSAWFRPHNRDKTIRNFYLVGAGTHPGAGIPGVVGSAKATAQVMLSDLAGA

SEQ ID NO: 198 - protein, 585 aa, Parasitella parasitica
  REGION 1..585: note = misc_feature - Pp.CrtI_A0A0B7NEC9, Parasitella parasitica
MSRKHIIIIGAGVGGTATAARLAREGFKVTVVEKNNFNGGRCSLIYHNGHRFDQGPSLYLMPKYFEDAFADLDERIEDHLELLRCDNNYKVHFDNGESIQLSSDLTRMKAELDRVEGPLGFGRFLDFLKETHIHYEKGTMIALKRNFESIWDLIRIKYAPEIFRLHLFDKIYSRASKYFKTKKMRMAFTFQTMYMGMSPYDAPAVYSLLQYTEFAEGIWYPRGGFNMVVQKLEAIAKKYGAEFMYDAPVSEINTIDGSKHVTGVTLECGRVIEADAVVCNADLVYAYHNLLPPCKWTQNTLASMKLTSSSISFYWSMSTKILQLDVHNIFLAEAYKESFDEIFKDFTMPSEASFYVNVPSRIDPTAAPAGKDSIIVLVPIGHIKSKISDASSENYSKMVDRARKMVLQVIECRLGMSNFADLIEHEQVNDPSVWQNKFNLWRGSILGLSHDVFQVLWFRPSTKDSTGRYDNLFFVGASTHPGTGVPIVLAGSKLTSDQVVRSFGMTPKPRKIEIKNRKALPEHDDLLFSWSSRYLAWQQLIFWGIFLFFYFFPQSDNGVTPASFINSFLPDVFHGDNSNIHQKIM

SEQ ID NO: 199 - protein, 494 aa, Paracoccus zeaxanthinifaciens
  REGION 1..494: note = misc_feature - Pz.CrtI_P94790, Paracoccus zeaxanthinifaciens R1534
MSSAIVIGAGFGGLALAIRLQSAGIATTIVEARDKPGGRAYVWNDQGHVFDAGPTVVTDPDSLRELWALSGQPMERDVTLLPVSPFYRLTWADGRSFEYVNDDDELIRQVASFNPADVDGYRRFHDYAEEVYREGYLKLGTTPFLKLGQMLNAAPALMRLQAYRSVHSMVARFIQDPHLRQAFSFHTLLVGGNPFSTSSIYALIHALERRGGVWFAKGGTNQLVAGMVALFERLGGTLLLNARVTRIDTEGDRATGVTLLDGRQLRADTVASNGDVMHSYRDLLGHTRRGRTKAAILNRQRWSMSLFVLHFGLSKRPENLAHHSVIFGPRYKGLVNEIFNGPRLPDDFSMYLHSPCVTDPSLAPEGMSTHYVLAPVPHLGRADVDWEAEAPGYAERIFEELERRAIPDLRKHLTVSRIFSPADFSTELSAHHGSAFSVEPILTQSAWFRPHNRDRAIPNFYIVGAGTHPGAGIPGVVGSAKATAQVMLSDLAVA

SEQ ID NO: 200 - protein, 578 aa, Rhizopus azygosporus
  REGION 1..578: note = misc_feature - Ra.CrtI_A0A367JJ19, Rhizopus azygosporus
MSKHIVIIGAGVGGTATAARLAREGFRVTVVEKNDFSGGRCSLIHHDGYRFDQGPSLYLMPKLFEDAFADLDERVEDHLELLRCENNYKIHFDDGESIHLTSDLTRMKIEMERVEGPEGFGRFLDFLRETHVHYQKGTWIALKKNFESIWDLIRLKYAPEILNLHLFDKIYRRASLYFKTKRMRMAFTFQTMYMGMSPYDAPAVYSLLQYTEFAEGIWYPRGGFNQVVQKLEFIASQKYRATFRYNAPVAKINTDGGRVCGVTLENGEVIEADAVVCNADLVYAYHKLLPPCSWTTKTLASKKLTSSSISFYWSMNKKISQLDVHNIFLAESYKESFDEIFKQYGLPSEASFYVNVPSRVDPSAAPDGKDSVIVLVPIGHMKSQTGEGEDYANLVNRAREMILSVIQARLGIKDFASMIEHEIVNDPKTWQAKFNLWRGSILGLSHNVFQVLWFRPSTRDTTNRYRNLFFVGASTHPGTGVPIILAGSKLTSDQVCKHFGKTPLPRKLEYTPQAKDYVEKSHDGLSWFSIFAVVVFLLSLFFAFFPEQSTGHTAASFINSCLPKPFQVDGTSSSFWVA

SEQ ID NO: 201 - protein, 578 aa, Rhizopus azygosporus
  REGION 1..578: note = misc_feature - Ra.CrtI_A0A367JYF3, Rhizopus azygosporus
MSKHIVIIGAGVGGTATAARLAREGFRVTVVEKNDFSGGRCSLIHHDGYRFDQGPSLYLMPKLFEDAFADLDERVEYHLELLRCENNYKIHFDDGESIHLTSDLTRMKAEMERVEGPEGFGRFLDFLRETHVHYQKGTWIALKKNFESIWDLIRLEYAPEILRLHLFDKIYRRASLYFKTKRMRMAFTFQTMYMGMSPYDAPAVYSLLQYTEFAEGIWYPRGGFNQVVQKLEFIASQKYRATFRYNAPVAKINVDGGRVSGVTLENGEVIEADAVVCNADLVYAYHKLLPPCSWTTKTLASKKLTSSSISFYWSMNKKISQLDVHNIFLAESYKESFDEIFKQYGLPSEASFYVNVPSRVDPSAAPDGKDSVIVLVPIGHMKSQTGEGEDYADLVNRAREMVLDVIQARLGIKDFASMIEHEIVNDPKTWQAKFNLWRGSILGLSHNVFQVLWFRPSTRDTTNRYCNLFFVGASTHPGTGVPIVLAGSKLTSDQICKHFGKTPLPRKLEYAPQAKDYVEKSHNGPSWLSIFAVIVFLLSLFFAFFPEQSTGHTAASFINSYLPKPFQVEGTSSSFWVA

SEQ ID NO: 202 - protein, 518 aa, Rhodobacter azotoformans
  REGION 1..518: note = misc_feature - Ra.CrtI_G3FHJ1, Rhodobacter azotoformans
MPATKHVSDADHAIVIGSGLGGLSAAMRLGAKGWRVTVIDKLDVPGGRGSSITQEGHRFDLGPTIVTVPQSLRDLWATCGRDFDKDVALKPVDPFYEVRWPDGSDFTVRQSTEAMKAEIARLSPGDVAGYETFLKDSEKRYWFGYENLGRRPMHKLWDLIKVLPTFGMLRADRSVYQHAARRVQDERLRMALSFHPLFIGGDPFNVTSMYILVSQLEKEFGVHYAIGGVAAIAQAMAQVIEGQGGAFRMNTEVDEIVVENGEAKGVRLASGEVLRAGLVVSNADAGHTYMRLLRNHKRKRWTDAHVKSRRWSMGLFAWYFGTKGTKGMWSDVGHHTIINAPRYKGLVEDIFLKGKLAKDMSLYIHRPSITDPTVAPEGDDTFYALSPVPHLKHANPVDWQAMGEPYRQSVLEVLEESMPGIGDRIGPSLVFTPETFRDRYLSPWGAGFSIEPRILQSAWFRPHNISEEVKGLFLVGAGTHPGAGVPGVIGSAEVMAKLAPDAPRARREAEPAERLAAE

SEQ ID NO: 203 - protein, 524 aa, Rhodobacter capsulatus
  REGION 1..524: note = misc_feature - Rc.CrtI_P17054, Rhodobacter capsulatus
MSKNTEGMGRAVVIGAGLGGLAAAMRLGAKGYKVTVVDRLDRPGGRGSSITKGGHRFDLGPTIVTVPDRLRELWADCGRDFDKDVSLVPMEPFYTIDFPDGEKYTAYGDDAKVKAEVARISPGDVEGFRHFMWDAKARYEFGYENLGRKPMSKLWDLIKVLPTFGWLRADRSVYGHAKKMVKDDHLRFALSFHPLFIGGDPFHVTSMYILVSQLEKKFGVHYAIGGVQAIADAMAKVITDQGGEMRLNTEVDEILVSRDGKATGIRLMDGTELPAQVVVSNADAGHTYKRLLRNRDRWRWTDEKLDKKRWSMGLFVWYFGTKGTAKMWKDVGHHTVVVGPRYKEHVQDIFIKGELAEDMSLYVHRPSVTDPTAAPKGDDTFYVLSPVPNLGFDNGVDWSVEAEKYKAKVLKVIEERLLPGVAEKITEEVVFTPETFRDRYLSPLGAGFSLEPRILQSAWFRPHNASEEVDGLYLVGAGTHPGAGVPSVIGSGELVAQMIPDAPKPETPAAAAPKARTPRAKAAQ

SEQ ID NO: 204 - protein, 545 aa, Rhizopus delemar
  REGION 1..545: note = misc_feature - Rd.CrtI_I1CU28, Rhizopus delemar
MDERKHVVIIGAGVGGTATAARLARQGFRVTVIEKNGFSGGRCSTLRHDGFRFDQGPSLYLMPKVFEEAFADLDERIGDHVDLLRCENNYRLHFPDGDTIELSSDLCRMQQVMDRIEGPGGMDRFLGFLQEAQVHHARGTPIAIERNFERIWDLVRYGSEIGRLHLLDTIYRRASRYFKTKKMRMAFTFQTMYMGMSPYDAPAVYSLLQYTECVEGIWYPRGGFHQVVHRLEQIAQEKFGASFVYNVPVAKINTQGRRVTGVTLADGEVIQADAVVCNADLVYAYHQLLPPCAWTTQSLAAKQLTCSSISFYWSMSRTIPALDVHNVFLAEAYQASFDEIFCQHRLPSQPSFYVHVPSRLDPSAAPPNKDAVVVLVPIGHMHKDPVDYDRLVQRARSTVLDVLHRRLGLEADWIEHEIVNDPRTWQAKFNLWKGSILGLSHHLFQVLWFRPSTKDASGRYDNLYFVGASTHPGTGVPIVLAGSKLTSDQVCRGFGQVPLPRKNPSRPPSLRPPLRPLWILLVLGFLGFLCSLTDQHLPLSFSRRQ

SEQ ID NO: 205 - protein, 513 aa, Rubrivivax gelatinosus; identical to SEQ ID NO: 172
  REGION 1..513: note = misc_feature - Rg.CrtI_Q840T3, Rubrivivax gelatinosus S1

SEQ ID NO: 206 - protein, 578 aa, Rhizopus microsporus
  REGION 1..578: note = misc_feature - Rm.CrtI_A0A2G4SFW1, Rhizopus microsporus ATCC 52813
MSKHIVVIGAGIGGTATAARLAREGFRVTVVEKNDFSGGRCSLIHHGGYRFDQGPSLYLMPKLFEDAFADLDERVEDHLELLRCENNYKVHFDDGESIHLTSDLTRMKTEMERVEGPEGFGRFLDFLRETHVHYQNGTWIALKKNFESIWDLIRLEYAPEILRLHLFDKIYRRASLYFKTKRMRMAFTFQTMYMGMSPYDAPAVYSLLQYTEFAEGIWYPRGGFNQVVQKLESIATQKYRATFRYNAPVAKINTDSGRVCGVTLENGEVIEADAVVCNADLVYAYHKLLPPCSWTTKTLASKKLTSSSISFYWSMNKKIPQFDVHNIFLAESYKESFDEIFEQYGLPSEASFYVNVPSRVDPSAAPDGKDSVIVLVPIGHMKSQTGEGEDYADLVNRAREMVLSVIQSRLGIKDFASMIEHEIVNDPKTWQAKFNLWRGSILGLSHNVFQVLWFRPSTRDTTNRYRNLFFVGASTHPGTGVPIVLAGSKLTSDQVCKHFGKTPLPRKLGYTPQPKDYVEKSHGEPSWFSIFAVIVFLLSFFFAFFPEQSTGHTAASFINSYLPKPFQVEGASSSFWIV

SEQ ID NO: 207 - protein, 507 aa, Rhodospirillum rubrum
  REGION 1..507: note = misc_feature - Rr.CrtI_Q2RX47, Rhodospirillum rubrum ATCC 11170
MKLHPAPTVPTQDRRPHAVVIGSGFGGLAAAVRLGARGYRVTVLEKLGGPGGRGGVFKQDGFTFDAGPTIVTLPSLFEELWTLCGRQMSDDVDLRPLTPFYTIRFNDGETFRAQSDPEAMRAEVARLSPGDVEGYERFIAESEDIYKVGFEQMADKPFSSITDMLKVVPQMLKLGALRSVWGHVAKHVADPRLRIALSFHPLFIGGNPLSVTSIYSMIAFLERRWGVHFAMGGTHSLVKGLVGLIEGQGNHLRYNAGVAEILVKNDRACGVRLETGEEIAADIVVSNADSAATYKNLLPAAHRRWWSDRRLAWSRYSMSLFIWHFGTKGTYPDVAHHTILMGPRYHTLIADIFQAKTLAKDFSLYLYRPTATDPSMAPEGCDSFYVLSPVPHLGGGQDWALEAEPYRKAILAELERTVLPGLGAKIVTSKVVTPVDFRDGLSSLNGAAFGLEPAFTQSAWFRPHNKSEEIDRLYLVGAGTHPGAGLPGVLSSAKILDTVVPDASVLV

SEQ ID NO: 208 - protein, 574 aa, Rhizopus stolonifer
  REGION 1..574: note = misc_feature - Rs.CrtI_A0A367KGJ1, Rhizopus stolonifer
MSQQKHIVVIGAGVGGTATAARLSREGFKVTVVEKNDFSGGRCSLIHHNGHRFDQGPSLYLMPKLFRDAFADLDERIEDHLNLLRCDNNYKVHFDDGQSIQLSSDLTRMKTEIERWEGPDGFGRFLDFMKEMHVHYETGVWIAIKRNFESIWDLIKIKYLPEVFRLHLFNKIYQRASVYFKSKRMRMAFTFQTMYMGMSPYDAPAVYSLLQYTEFAEGIWYPRGGFNMVVQKLESIAANKYGAKFMYNAPVAKINTVDGQKRVTGVTLESGEVIEADAVVCNADLVYAYHHLLPPCSWTTQTLASKKLTSSSISFYWSMSKVIEQLDVHNIFLAEAYKESFDEIFKDLSMPSEASFYVNVPSRIDPSAAPANKDSVIVLVPIGHMKTSQKEDYAELVKRARKMVLEIINRRLGTKDFEQYIEHEIVNDPSVWQSKFNLWKGSILGLSHDVFQVLWFRPSTRDSTGRYDNLYFVGASTHPGTGVPIVLAGSKLTSDQVCKGFGQKPLARKLQLIEKTVEPAEREKSSFLLFALCFLLLVGLFAFFPEKSTGHTAASFINSHLPKSLQVILDANVI

SEQ ID NO: 209 - protein, 523 aa, Stigmatella aurantiaca
  REGION 1..523: note = misc_feature - Sa.CrtI_Q096M3, Stigmatella aurantiaca DW4/3-1
MVRHVIVVGSGPGGLSAAINLAGQGLKVTVVEKDAVPGGRMKGLTLGARGEYALDTGPSILQLPGVLERIFVRSGKRIEDYVKLVPVDPNTRVHFWDGTHMDTSKNLAHMEAELAKFSPALPRALKAWMEEGKEKYAIAYEKFIATNAGSLGYYAPWRLASTLRFKPWQTLYRHLDSFFHDDRVTYALAYPSKYLGLHPTTCSSVFGVIPFLELAFGVWHVDGGFRALARGMKRCAEDLGATFRMGEAVEQVLVDGGRAVGVRLANGKRLEGDAVVVNADLAYAAQSLLPADVREGTRLSDASLERAKFSCSTFMAYYGLDRVYPELPHHLIYLSENARRTDRDALEDRVLDLEDPPFYVCNPCVTDPSGAPEGHSTLYVLVPTPNTSQPVDWAATERVLRERIPDMLAKVGLKDVRRHIRAERYFTAETWRDDFHVFRGAVFNLSHTWMQLGPMRPKVKSPDVQGLYWVGGGTHPGSGLLTIMESANIAADYLTREAGKGPLPQWPYVPPLEEAPVTQARTG

SEQ ID NO: 210 - protein, 507 aa, Streptomyces griseus
  REGION 1..507: note = misc_feature - Sg.CrtI_P54981, Streptomyces griseus NCBI 3933
MITVKGPVDHVVVVGAGLAGLAAALHLLGAGRRVTVVEREDVPGGRAGLLESGGFRIDTGPTVLTMPDLVEDAFAAVGERMADRLELIRLAPAYRARFADGSQLDVHTDGAAMEAAVEEFAGARQAVGYRRLRIWLERLYRVQMRRFIDTNFDSPLQLAHPDLARLAALGGFGRLDARIGHFVSDERLRRVFSFQALYAGVPPARALAAYAVIAYMDTVAGVYFPRGGMHALPRAMADAASDAGAVLRYGQRVTRLERSGDRVTAVVTDQEHIPCDAVVLTPDLPVSYRLLGRTPHRPLPLRHSPSAVILHTGTDRTWPDLAHHTISFGAAWKNTFHELTRTGRLMSDPSLLITRPTATDPSLAPPGKHLHYVLAPCPNTEVGPGVREWRELGPRYRDELLAELERREMPGLGAAIEEEGLVTPVDWTAQGHAAGTPFSVAHTFPQTGPFRPRNLVRGTVNAVLAGCGTTPGVGVPTVLISGKLAAERITGPRIARAPRSAPGGSSA

SEQ ID NO: 211 - protein, 572 aa, Syncephalastrum racemosum
  REGION 1..572: note = misc_feature - Sr.CrtI_A0A1X2H2F7, Syncephalastrum racemosum
MSRQKHIIVIGAGAGGTATAARLAREGYRVTVVEKNDFNGGRCSLIHHNGHRFDQGPSLYLMPKLFEECFSDLDERIEDHLDLLRCDNNYKVHFDDGDSIHLSSDLTRMKREAERIEGEDGFSRFLEFMKETHVHYERGTFLAIKRNFESIFDMVKLKYAPEILRLHLFDQIYARAAKYFQTKKMRMAFTFQTMYMGMSPYDAPAVYSLLQYTEFAEGIWYPRGGFNMVVQKLEAIAQKSGAVFKYESPVAKINTADQDRRVTGVTLQDGTVLPADAVVCNADLVYAYHKLLPPCRWVRTTLASKKLTSSSISFYWSMKQKILQLDVHNIFLAEAYKESFDEIFQDHTLPSEPSFYVNVPSRIDSSAAPADKDSVIVLVPIGYMRSQDGNCDVDYQILVKRARTMVLEVLHRRLGLKNFAELIEHEIVNDPSVWQQKFNLWRGSILGLSHDVLQVLWFRPSTKDSTGRYDNLFFVGASTHPGTGVPIVLAGSKLTATQVCQYFGKKGLTPSKLLSKATFEYAPEGDTAFTNHLVYMLLSLFSMGMTPAAFINACLPSPFRVVIAAAAEEIQI

SEQ ID NO: 212 - protein, 454 aa, Sulfolobus solfataricus
  REGION 1..454: note = misc_feature - Ss.CrtI_Q97UT6, Sulfolobus solfataricus P2
MMKAVVVGAGLGGISTAILLKKFFDEVVVLEKNTTPGGRARYFSVGEFRFDMGPSWYLMPEIFEEFFSEAGENPHPIIRVNPPVKVMESKFLKKEYREFTGDNVDDRYLNKTRLFYDFSKKFIKKEMVFSDFLDKEVLVNISALFDTLDNFNARYFRDSLMRKAMGFSSVFIGGSPFRVPAVYAMINYAIYGQGVFYPYKGFEGYVKRLYEIARKKGVEFKFDFPVNKVKIDEGKVVEVSSNIKREMGDVFVFNMDYFYADSLLPEEYKVISGRRYKLSPSAILAYIGLEDTLDLPHHTVIINGDWREHFTSIDRNSLPSVENTSYYVSYRGATNKETRDLVILIPVGTGTEISKELIDGVIRDLEVKIGCKIKASYIRIYGPDDFKRDYNAYEGTAFGLAHTLDQTGPFRLPMKNTRLKNLYYVGQYTQPGIGVPMVTLSSMIVSKKISKEFL

SEQ ID NO: 213 - protein, 584 aa, Thamnidium elegans
  REGION 1..584: note = misc_feature - Te.CrtI_A0A8H7VTL0, Thamnidium elegans
MSTQKHIVVIGAGVGGTATAARLTREGFRVTVVEKNSFSGGRCSLIHHNGHRFDQGPSLYLMPKMFEDAFADLDERVEDHLELLRCDNNYKVHFDDGESIQLSSDLTRMKSELDRIEGPLGFGRFLDFLKETHIHYERGVFIAIKRNFESIWDMLQVKFAPEVVRLHLFGKIYDRASKYFLTKKMRMAFTFQTMYMGMSPYDAPAVYSLLQYTEFAEGIWYPKGGFNMVVQKLEAIAKNKYGAKFLYDSPVSKINTEGNRATGVTLDSGEFIEADAVVCNADLVYAYHHLLPPCKWTMNTLAAKKLTSSSISFYWSMKEKVPKLDVHNIFLAEAYQESFDEIFNDHSLPSEASFYVNVPSRIDPSAAPEGKDTVIVLVPIGHMKSLAGNAEEADYPKMVERARAMVLEVIEHRLGMKNFSDLIEHESYNDPAIWQSKFNLWRGSILGLSHDIFQVLWFRPSTKDSTGRFDNLFFVGASTHPGTGVPIVLAGSKLTSDQVTTSFGKRPLPRKVQDQISYKAEDVTHGVQLMPLFVYTFVITFLFFVFFPTNATNQTAASFINSALPKPFRVSYENDLLLERLNAV

SEQ ID NO: 214 - protein, 584 aa, Verticillium dahliae
  REGION 1..584: note = misc_feature - Vd.CrtI_G2WRY7, Verticillium dahliae
MQKSGEKPRKAIVVGAGAGGVAIAARLAKAGYKVTVLEKNDFTGGRCSLIHHEGYRFDQGPSLLLLPQLFHETFRDLDTTIADSGVDLLRCQDVNYNVWFHDGELFKHSSDLATMKVEVERWEGKEGFARYLSWMREAHTHYEVSVTDVLHRNFTSLFNLARPSLLKHVVALHPLESIYARASRYFWTERLRRVFTFAVMYMGMSPFDAPATYSLLQYTEFAEGIWYPRGGFHAIIAALVRIGEQMGVEYRLNTSVASISTAEESNRATGVVLESGESLSADVVVINADLVYAYSNLMPKTPSTVAYSQSLQKKEASCSSISFYWSVNRKIPELGTHNIFLAEEYRESFDAIFKRHGLPDQPSFYVNVPSRIDPSAAPEDGDAIIVLVPIGHLARSKGGVDAPGLPTDEKSWINLVARARAAVLETISARTGCAPIGQFITNEIVNNPLSWEDKFNLDKGAILGLTHSVPNVLAFRPRTRAEGLENAYFVGASTHPGTGVPIVLAGAKITAEQILDDRGLPVPWAHTKNFPKQQLAERRQKTKALDLMRYTFHFGSEEVAVLSFLFAVCMMYLFVWPRVVSLVC

SEQ ID NO: 215 - protein, 537 aa, Vibrio harveyi
  REGION 1..537: note = misc_feature - Vh.CrtI_A7MVU2, Vibrio harveyi ATCC BAA-1116
MAHNHSEVSGRAIVIGAGFGGIASALRFCAKGYSVTLVDKGDQLGGRARVFERNGFIFDAGPTVITAPFLIDELFALFGKKTSQYVSIVPVTPWYRFYFHDNTTFDYGGTLEATLEEIKKISPNDVIGYQNLLATSESIFDIGFTQLSDEPFHNPLSMIRQVPNLIRLGCYKTVWQLVCEHLEHEKLRQAFSIQPLLVGGNPFDTTSIYNLIHFLELKWGVHFAIGGMGALVNGLKKLMLENGIDIQLNTEVKSVVSKNGKVSGIETSNGDLMDCDVVVSNADPAFLYRKMIAKKDQTLSARLKSRHAKFSMGLFVLYFGTTQKYEDVAHHTIWLGKRYESLLNDIFHDKVLADDFSLYVHRPTATDPNMAPEGCDSFYVLAPVPNLESNVDWETVKNKYSDKIVNALSETLLPGLSDCIVEQFCMTPSDFQCDYNSINGAGFSIAPTLTQSAYFRFHNRGEGLKNLFVVGAGSHPGAGLPGVLCSAKVLDKIVPEFTGDDARVRSCGMPLTNTDTTKPRMNLPSRLSGDNHGKYGS

SEQ ID NO: 216 - protein, 504 aa, Xanthobacter autotrophicus
  REGION 1..504: note = misc_feature - Xa.CrtI_Q93CI7, Xanthobacter autotrophicus Py2
MMLDATKTIRRAAVIGSGFGGLSLAIRLQAAGIRTTVFEQRDKPGGRAYVYEQDGFTFDGGPTVITDPTCLEEVFAAAGRKLSDYVELMPVSPFYRLLWPDGRQFDYVNDQAALDAQIATFDKADVEGYRRFLAYSQAVFEEGYLKLGAVPFLEFSSMMKAAPKLVRLEAWRSVYAMVSRFIRDDHLRQAFSFHSLLVGGNPFSTSSIYALIHALERKWGVFFPRGGTGALVRGMVKLFTDLGGEIRLTSPVDEIVVEGQRATAVKLKSGETLPFDLVASNADVVHTYRHLLRGAARGRSEGARLAKTRHSMSLFVTYFGARRTWDHLQHHTVLFGPRYRGLVDEIFKGPNLPDDFSLYLHAPTVTDKSLAPEGCTAFYVLSPVPHLGKADIDWEVEGPLYRDRILAHLEERLLPGLRDSLVTSRILTPFGFRDELSAHQGSAFSVEPLLTQSAWFRPHNRDAKIANLYFAGAGTHPGAGVPGVVGSAKATAGLILADLGVTPR

SEQ ID NO: 217 - protein, 594 aa, Arthroderma gypseum
  REGION 1..594: note = misc_feature - Ag.CrtYB_E4UPP6, Arthroderma gypseum
MGLDYLMVHVKYNLPPALLLTILYKPFFTRLEVHKIVLLCTIAVVWTIPWDSYLIRTRIWSYPADSVLGQTLFQIPLEEVFFFIIQTYNTSLLYIIFNKRLVLPSYLSGPTKPLAQGLFGPITHRTQRDLGTLFFTGILILGISFIYIGGEYMYLGLILSWVSPILVMQWVLMYRFLLALPPASVWVPIALPTLYLWVVDTLALRRGTWVIESGTKVDIQLWEGLEIEEALFFLVTNVMVVFGIAAMHNAAALFEYKAFISTTAMGDTPSIYQLITLFLTSSRLYDTNVLQEMSQAVTLLKQKSQSMYLGSAMFEGQLRLDLIALYSFCRKADDLIDDAPDRETAKYWIEQCEKALELRFKLKETALDDTEAYQLLTKSIPQPLHAAAHLLPASRLPKEPLSCLLQGFEIDLKFDFEKGSFPIATEHDLEVYAYHVAGTVASLLLELVFRHHPVSISEAERLRVISAGEVMGRALQYTNIARDITRDAEIGRVYIPSSWLAEEGLTPSMVISHPRNSKLIPLRRRILEKADKCYCETQEAISKLPSNVQGPVRATVTAYMEIGQVIRENETKIWNGKLKVSRWRRFKRAWLAML

SEQ ID NO: 218 - protein, 582 aa, Aspergillus niger
  REGION 1..582: note = misc_feature - An.CrtYB_A2QM49, Aspergillus niger
MNQNGTRLCYSVREVPPTSFVSAPANYKRASSHCTYTIPAASALTVLYYPFFTAQDRCKICILITIAILATLPWDSYLIRSAIWTYPPDAVVGLKILDIPIEEVFFFAIQTYITSLTYCIFTKPLVRPMYLRSHLERRGTRYVVATVILALMGGGTACLLLGRRMTYLGLILVWACPILLFQWMMSYPFLSELPWKPTITSICLPTLHLWFADSRAMGTGTWRIEEGTKLNFRIGGLELEEALFFLVSNMMVVLGLVGCDYAYALQEYESLSQPASDVYITLRKALSLLARPLPIDASLISALSQAVYRLQEKSQSMFLGSALFQGQLRIDLIFLYSFCRVMDDLIDEAEDEQEARFWVTECRHLLDSTHRSEPHSDHFYTGKKGEEHERLRQSISYLPPSHLSNDSFDDLLKGFEIDLKFNPQREAFPIQSEYCLDQYAGFVAGTVGVLVFDLTLFHCGHYFIQDVPRLRRAAKDMGKAMQCQPRGDGGATSGRKRAIAGNRGELYGYWTTAEGAERHKPGASVEDEGAFGATAKSWLAGDVITELCHCFIQAEYVIYLVRHQNASADTLVLLSALVYRLE

SEQ ID NO: 219 - protein, 587 aa, Aspergillus oryzae
  REGION 1..587: note = misc_feature - Ao.CrtYB_Q2U4X9, Aspergillus oryzae
MGLDYILVHVTYNIPLAGILTLVYWPFMTRLDWQKISTLVIISLVATIPWDSYLVRHRIWTYAPNGVIGWTLYDIPSEEVFFFIIQTYNTSLVYLILTRWLVLPMYLGTVARKETLIGASILLLAISVGLIALCFGDHFTYFGMIITWAGPFLLIQWVFSSGFIIALPKLELMVSITLPTLFLWTVDTISINQGTWTVEAPTKLGVQLWSGMDIEEVLFFLITNIVIVFGLVCIDYAIAMATCELVQSPQAVQSFPSYFRVLARFVTNKYHPDKQFVASLRKAVDRLAASSQSMYMGSAMFQGPFRIDLILLYSFFRVADDLVDESQDTESARMIIEQCDQLLEAKFSHPELFPFSPGYQEAKHPAPPELIAAIDSLPVSRLRLEHLKGLIEGFRTDLTFSAKPGSFPFVTESDLDTYAYHVASSVAASMLGLVVHHFPDHQFAINVFLRRRVVDAGERMGQTLQYINVARDIARDAAINRVYLPTTWLKQQGLGPEDVLASPTDSRLELVRDRLLDRAEFLSASAREEMKFLPDEVQGPFLATVDSYLEIGAALRRGMRPRTLDDKLRLPLGTRLWVAYRAMAWRK

SEQ ID NO: 220 - protein, 608 aa, Blakeslea trispora
  REGION 1..608: note = misc_feature - Bt.CrtYB_Q67GH9, Blakeslea trispora
MSILTYLEFHLYYTLPVLAALCWLLKPFHSQQDNLKYKFLMLMAASTASIWDNYIVYHRAWWYCPTCVVAVIGYVPLEEYMFFIIMTLMTVAFSNFVMRWHLHTFFIRPNTSWKQTLLVRLVPVSALLAITYHAWHLTLPNKPSFYGSCILWYACPVLAILWLGAGEYILRRPVAVLLSIVIPSVYLCWADIVAISAGTWHISLRTSTGKMVVPDLPVEECLFFTLINTVLVFATCAIDRAQAILHLYKSSVQNQNPKQAISLFQHVKELAWAFCLPDQMLNNELFDDLTISWDILRKASKSFYTASAVFPSYVRQDLGVLYAFCRATDDLCDDESKSVQERRDQLDLTRQFVRDLFSQKTSAPIVIDWELYQNQLPASCISAFRAFTRLRHVLEVDPVEELLDGYKWDLERRPILDEQDLEAYSACVASSVGEMCTRVILAQDQKENDAWIIDRAREMGLVLQYVNIARDIVTDSETLGRCYLPQQWLRKEETEQIQQGNARSLGDQRLLGLSLKLVGKADAIMVRAKKGIDKLPANCQGGVRAACQVYAAIGSVLKQQKTTYPTRAHLKGSERAKIALLSVYNLYQSEDKPVALRQARKIKSFFVD

SEQ ID NO: 221 - protein, 587 aa, Colletotrichum graminicola
  REGION 1..587: note = misc_feature - Cg.CrtYB_E3Q717, Colletotrichum graminicola
MGYDYALVHVKYTIPLAALLTVFSYPVFTRLDVVRTLFIVTIAFVATIPWDSYLIRTNVWTYPPDAVLGPTLYDIPAEELFFFIIQTYITAQLYIILNKPVLHAQYLNSPATLPQWIKSGKLVGQLALSGSVLLGTWLIAKKGEGTYLGLILVWACTFALFTWTITAHFLLALPLACTALPILLPTVYLWIVDEMALGRGTWAIESGTKLELQLFGSLEIEEATFFLVTNMLIVFGIAAFDKAVAVCDAFPEKFDKPADALAMSLLRARVFPSSKYDMQRILGIRQAAARLAKKSRSFHLASSVFPGRLRIDLTLLYSYCRLADDLVDDAATPEEAAVWISKLDRHLSLLYKDPDATSTPLASKYAAENFPPSALSALDMLPTSLLPREPLAELLKGFEMDLSFSNSAFPIADPEDLELYAARVASTVGQACLELVFCHCQHGLPDYMKAYLRNTARQMGLALQFVNISRDIAVDAKIGRVYLPTTWLKEEGLTPEDVLKSPNSEGVGKVRRRILAKALDHYGEARDSMKWIPSEARGPMIVAVESYMEIGRVLMRNGGSAAADGSGRATVPKSRRIWVAWSTLMAA

SEQ ID NO: 222 - protein, 612 aa, Gibberella fujikuroi
  REGION 1..612: note = misc_feature - Gf.CrtYB_Q8X0Z1, Gibberella fujikuroi (F. fujikoroi)
MGWEYAQVHLKYTIPFGVVLAAVYRPLMSRLDVFKLVFLITVSFFWVVKGLEANSCRLLLFLPCMLILRGVLLRLTNADSPWDSYLIKNRIWTYPPGVVVGLTAWDIPAEELFFFVIQTFNTSLLYMILSKPTFHPIYLSKKTGWGKIAGQILFASAIIFGLVSVSSGGEGMYMGLILIWACPFLLFLWSISYQFIVNLPWTNTALPIALPTLYLWVVDTFALRRGTWSITSGTKYGVVLWDGLEIEEAVFFLLTNTLIVFGLIACDNNLAILDTFPEHFPRTKGVPSLLTIIRTLILPKEKYDEERIQGLVSAVALLRKKSRSFYLASGTFEGRLRIDLIRLYAFCRAADDLVDEAPSVDDSRASIEKLRKFLDLAYEENQEEPSQRLREYVTSSIPEMFHMALLQLPTYYLPKQPLDDLLKGFDTDLLFDRKSGAFPIETTEDLDIYGSRVAGTVAELCNHLILYHTPEAVPEDIQREVVASGQEMGIALQYVNIARDIKTDAEIDRVYLPLSWLKEAQLTPEDVIQQPHGPTIEALRHKLLDRAFEKYNMAKGAIDKLPSEGKGPIRVAVESYMEIGRVLREKGPTMKKGRATVPKMRRIRVAWSALNK

SEQ ID NO: 223 - protein, 581 aa, Leptosphaeria maculans
  REGION 1..581: note = misc_feature - Lm.CrtYB_E4ZUB5, Leptosphaeria maculans
MGFDYALVHLKYTIPPAVLLTLLYRPLLTKIDVYKVAFLVTIAVVATIPWDSYLIRNRIWSYPDHVIIGPTLFDIPLEEVFFFVVQTYNTSLLYLVLSKPTFQPVYLCTERDELHGSWRLKRLIGQAILLGAIAWGWFCVRERGLGTYTGLILIWAGPFLLLLWSLAYQFIIGLPFTNTLLPIVLPTLYLWIVDTLALRRGTWVISPGTKFGVHLWDGLEIEEALFFLLTNVLIVFGQLAFDNALAVLYAFPHLFPDPSLLPSPATLIRSLLTSCAQYDEARLTGFREAVSRLKRKSRSFYLASSTFQGPLRMDLLLLYSFCRVADDLVDNAATTEEARQWIAKLHKFLDNVYRKDVVCSSVTDQVRKEFPLDTHSALLQLPCCKLSAEPLRDLLRGFEMDLEFNSTSPIQSTEDLVLYSERVAGTVAQMCIQLIFHLYPSSLTAEKRHKVVAAGNSMGVALQYVNIARDIGVDAKIGRVYLPTDWLSEVGLNCDTVLKDPKDPRIEALRGRLLDDAFSFYEEAKLAIAQLPIEAQGPIRVAVESYMEIGRTLKQDGFIVKAGRATVPKWRRVLVAWRTLN

SEQ ID NO: 224 - protein, 606 aa, Mucor ambiguus
  REGION 1..606: note = misc_feature - Ma.CrtYB_A0A0C9MMT4, Mucor ambiguus
MEVHLYYTLPMLGVIFWLSKPYYTSTDSLKFKFLSLVAFTTASVWDNYIVYHKAWSYCPTCVTAVIGYVPLEEYMFFIIMTLLTVAFTNLVMRWHLHSFFIKPETPIMQSVLVRFVPITALLTTAYKAWHSAVPGNPLFYGSCILWYACPVLALLWFGAGEYMMRRPLAVLSSIALPTLFLCWVDVVAIGAGTWDISLATSTGIFVVPHLPVEEFMFFALINTVLVFGTCAIDRTMAIIHLFKKKSPYQRQYQNDKSFLHQILEMTWAFCLPDQALHTETFHDLSISWDILRKASRSFYTASAVFPGDVRQELGVLYAFCRATDDLCDNEQVPVQERKDQLTLTHRFVSDLFSQKKSAPTAIDWDFYNDQLPAPCISAFKSFTRLRHVLEADAIKELLDGYKWDLERRCITNQEDLNYYSACVASSVGEMCTRIILAHADKPTSRQETQWIIQRAREMGLVLQYTNIARDIVTDSKELGRCYLPQDWLADKEVGLIQDGRAREIGEERLLSLSHRLIYQADELMAVANKGIDKLPSHCQGGVRAACNVYASIGTKLKSYRHHYPSRAHVGNSKRVQIALLSVYNLYTAPIVTKQGRQGKMRNLNTI

SEQ ID NO: 225 - protein, 623 aa, Mucor circinatus
  REGION 1..623: note = misc_feature - Mc.CrtYB_A0A8H7S7S8, Mucor circinatus
MLTYMEVHLYYTLPILGVLFGLLRPFHSPQETFKYTFLCAMAFSTASIWDNYIVYHRAWSYCPSCVTAVIGYVPLEEYMFFIIMSLMTVAFTNMIMRWHLPSTFIKPDTPMLKSMLVRYLPIVGFLSVGVHAWNLAVPDTHLFYGSCILWYSCPVLALLWFGGGEYICRRYKAVLTSIIVPTIYLCWVDKVAIAAGTWHISLRTSTGVMVAPSLPLEEFMFFLLINVVLVFATCAIDRAQAILHLHGNELRGHPNNSKLSNLIPNQLTQLLYLTQAFCMSDQSLDTETYRDLHTTWKILKQASFSFYTASAVFPTHIRQDLGVLYGFCRATDDLADDEVASVEQRKTQLATVRRCIHDMFATKTPNMDWSLYDLPSSCIASLRAFTRLRPKLQVDAVNELLDGYDFDLDRSIVKNEADLERYSACVASSVGEMCTRVMLEYPETWTIERARDMGLVLQYTNIARDIVTDSQQLRRCYVPRDWLTEKESEAMTTGRAEQLGQTKLRSLALKLVYSADELATRARRGIDRLPFDCQGGVRAACSVYMAIGNELIKADGYPHRAHVSTWNRAWVVLKNVYQEAHSSSLQGFIRNSTSITNSKNNSVDDENKIATTRKGKQRAFCLL

SEQ ID NO: 226 - protein, 611 aa, Mucor circinelloides
  REGION 1..611: note = misc_feature - Mc.CrtYB_S2JTE4, Mucor circinelloides f. circinelloides
MLLTYMEVHLYYTLPMLGVLSWLSRPYYTSTDSLKFKFLSLVAFTTASVWDNYIVYHKAWSYCPTCVTAVIGYVPLEEYMFFIIMTLLTVAFTNLVMRWHLHSFFIKPETSVMQSVLVRFVPITALLIIAYKAWHLAVPGKPLFYGSCILWYACPVLALLWFGAGEYMLRRPLAVITSIALPTLFLCWVDVVAIGAGTWDISLATSTGKFVVPHLPVEEFMFFALINTVLVFGTCAIDRTMAILHLFKKKSPYQRQYQREKTFVHQILEMTWAFCLPDQVLNNETFHDLSVSWDILRKASKSFYTASAVFPGDVRQELGVLYAFCRATDDLCDNEDVPVQKRKDQLSTTHQFVSDLFSQKSSAPTAIDWDFYNDQLPASCISAFKSFTRLRHVLEADAIKELLNGYKWDLERRSIMNQEDLRYYSACVASSVGEMCTRIILARSDKAYSQQETQWIIQRAREMGLVLQYTNIARDIVTDSKELGRCYLPQDWLTEKEIGLIQNGRAREIGEARLLSLSHRLVYQADELMVVANKGIDRLPSHCQGGVRAACNVYASIGTKLKSYKQHYPSRAHVGNSKRVQIALLSVYNLYTAPIAIKQGRQGKMRNLNTI

SEQ ID NO: 227 - protein, 606 aa, Mucor plumbeus
  REGION 1..606: note = misc_feature - Mp.CrtYB_A0A8H7RIZ6, Mucor plumbeus
MVLTYMEVHLYYTLPMLGLLSWLSKPYYTSNDSLKFKFLTLIAFTTASVWDNYIVYHNAWSYCPTCVTAVIGYIPLEEYMFFIIMTLLTVAFTNLIMRWHLHSFFIKPETPILQSILVRFIPITGLLITAYKAWYLAVPGKSLFYGSCILWYACPVLALLWFGAGEYMLRRPLAVFVSIALPTLFLCWVDVVAIGAGTWDISLATSTGKFVVPHLPVEEFMFFALINTVLVFGTCAIDRTMAILHLFRKKNPYQSKYQQTEKTFLHQIVEMTWAFCLPDQALNTETFKDLSVSWDILRKASKSFYTASAVFPGDIRQELGVLYAFCRATDDLCDDESVSVNERKKQLKLTSQFVSDLFSQKVSAPSAISWEIYSDQLPASCISAFKSFTCLRHVLEVEAIKELLNGYKWDLERRSVLNQDDLRQYSACVASSVGEMCTRIILAHSDKFYTQQETQWIIQRAREMGLVLQYTNIARDIVTDSKKLGRCYLPHDWLSENDIVMIQNGYAREIGEEKLLLLSHRLIQQADELMLIANKGIEKLPSHCQGGVRAACNVYASIGAKLKLHKQQYPSRAHVSNLKRIKIALFSVYNLYNAPTTIKQNRQEKM

SEQ ID NO: 228 - protein, 602 aa, Mucor saturninus
  REGION 1..602: note = misc_feature - Ms.CrtYB_A0A8H7RF36, Mucor saturninus
MLTYMEVHLYFTLPVVGLLYFILKPFHSKQDNLKYQFLTAVAVTTASLWDNYIVYHKAWSYCPTCVVAVIGYVPLEEYMFFVIQTLMTVSFSNLVMRWHLPTFYIKPQVSVYQSLFVRFIPIFGLLTIAYKAWNVAIPGKSLFYGGCILWYVCPVLALLWYGAGEYILRRPLAVLTSIVVPTIYLCWVDRYAIRAGTWDISLQTSTGKMVVPHLPLEEFMFFALINTVLVFAACAIDRAQAILHLFKTESVYKTHTQAPLVQRMSELCWAFCLPDQMLNNETLQDLSISWDILKKASKSFYTASAVFSSYARQDLGILYGFCRATDDLCDDESVSVEKRKEQLNLTRRFVQDIFSQKTNMPVVDWEFYNRQLPSSCVSAFKTFTRLRHILHVDAIEELLEGYEWDLERKEVRTQDDLYKYSACVASSVGEMCTRILLFHDYRQSDVWIIARAREMGLVLQLTNIARDIVTDSQELGRCYLPMDWLTDNESSLIAAGKARELGDSRLLQLSKTLIRDAESIMKTANKGIEALPFHCRGGVRAACNVYAAIGHVILKERTHYPTRAHIKSSGRSKIALLSVYDLYKHKTEAPKSRQGKIRGFMV

SEQ ID NO: 229 - protein, 593 aa, Podospora anserina
  REGION 1..593: note = misc_feature - Pa.CrtYB_B2ATB0, Podospora anserina
MAYDYALVHLKYTIPLAALLTVIAYPIFHRIHFLQIGSLIVVSFLATLPWDSYLIRSNIWTYPPDAIIGPRLYGIPIEELFFFVIQTYITSLFYILLSKPLFHPLYLSTQRNPPQRIARGKVIGQGILVALTLYGVHQIRTGGPGTYLGLILAWAFPFALLTFTVAGRFILTLPLTSTVVPIIIPTVYLWLVDELALGRGTWAIESGTKLGWCLFGVLDIEEATFFLATNILIVFGMAVFDQYLAIIFAFPHLFPKVPRSPTPLMLVQSRFSNTKQYDLERIAGLSDAVTRLKAKSRSFYLANSLFTGRLRIDLILLYSFCRLADDLVDDSTSRTEVKSWTTKLYKFLDLHYKSDVKANKARINDYIDEAFPPEAKSALKYLPATILPSQPLYQLIEGFELDSQFSFHDSSESAKYPIVDEDKLNYYGQCVAGTVGELCVALIIEHCEPEMPDERKKMLMSVSRTMGVALQYVNIARDIVVDAEMGRVYLPTTWLKEEGLTPEDVVAHPRGKHVENLRRRLLSEAFKLYDEARPKMNGIPKEARGPMIGAVETYMEIGRVLRELEGGVELERGKATVPGGRRLKTVLKALFSA

SEQ ID NO: 230 - protein, 585 aa, Phaeosphaeria nodorum
  REGION 1..585: note = misc_feature - Pn.CrtYB_Q0V6M5, Phaeosphaeria nodorum
MGFDYAIVHVKYTIPPAVLLTLLYRPLFTKLDAFKVLFLVTVAVTATIPWDSYLIRTNIWSYPDHVVIGPTLLDIPLEEVFFFFIQTYNTTLLYLILSKPTFQPAYLRAGRPTASSPWKYQKLAGQLFLVGATVWAGLRVHENAKGTYTGLIVVWAAPIILLQWTLAYQFILGLPWTNTVLPIAIPTLYLWLVDTLALRRGTWVISPGTKYGVHLWDGLEIEEALFFFVTNTLIVFGQLAFDNALAVLYTFPALFPKPPSMPTPLDLINALWVSPYKYDRARLAGLQDAVLRLKRKSRSFYLASATFPGPLRSDLLLLYSFCRVADDLVDNAATAEEAKEWISKLHQYLDLVYSDAKSSTVSEDFVQAHFPSDARSALLQLPAHKLPRQPLQDLLHGFEMDLAFNTSSPIKTETDLRLYSERVAGTVAQMCIELIFRLYPSNMTSGEERKVVDAGNQMGMALQYVNIARDISVDAHIGRVYLPLDWLQESGLTYDEVLISPEGARMESLRMRLLEKAFSIYDGARGAIETLPVEARGPIRVAVESYMEIGRTLRQKGYTVRAGRATVSKWRRVIVAWRTLNKSIA

SEQ ID NO: 231 - protein, 583 aa, Pyrenophora tritici-repentis
  REGION 1..583: note = misc_feature - Pt.CrtYB_B2WAQ3, Pyrenophora tritici-repentis
MGFDYALVHLKYTIPPAVLLTWLYRPFFTKLDVYKVGYLVSIAVASTIPWDSYLIRTGIWSYPTHVIIGPKLCDIPLEEVFFFIIQTYNTSLLYLLLSRPTFQPVYLNTERGAARRQWRYMRLAGQVFFLALIAWGWRCIRHGGLGTYTGLILVWAGPFLLMLWSLAYQFILALPVTNTALPIFLPTLYLWVVDTLALRRGTWVISTGTKYGLHLWDGLEIEEALFFLATNALIVFGQLAFDNALAVLYTFPHLFTGPSLLPSPVLLMRALLTPCSKYHDARIKGLDEAVNRLKRKSRSFYLASATFPGPLRADLLLLYSFCRVADDLVDNASDADEARAWIAKMRKFLNNVYSDKLPQSVVHSQICDDFPPSTQSALLQLPATKLSPQPLEDLLHGFEMDLAFQQGPIIRTMEDLRVYSERVAGTVAQMCIQLIFYWYPSTLDTEEKNVIVAAGNSMGVALQYVNIARDIEVDAQIGRVYLPLNWLSEAGLSYDDVLKKPNQAQIQTLRKHLLNHAFSVYEKAKDSIERLPIEARGPIRVAVESYMEIGRILRSEQYQVKAGRATVPKSRRIMVAWRTLNSK

SEQ ID NO: 232 - protein, 568 aa, Rhizopus azygosporus
  REGION 1..568: note = misc_feature - Ra.CrtYB_A0A367JYH9, Rhizopus azygosporus
MLTYMEVHLYYTLPVLGILFWLLRPFDSKEDRFKYQFLACIAFFTASVWDNYIVYHKAWSYCPTCVVAVIGYVPLEEYMFFIIMTFMTVSFTNLVMRWYLPSTFIRPQTPLIQTWFVRGVPIIVLMTIAYKAWKLAVPAKPLFYGACILWYVCPVLAILWYGSGEYMLQRPLSVILSITLPTLYLCWVDKIAIAAGTWHISLRTSTGKFVSSDLPLEEFMFFTLINTVLVFATCTIDRAQAVAQLFNKYPQNTVDQLKMLLWAYLVPDQMLNPQVINDLSVTWDILKRASKSFHTASVVFNYAVRQDLSVLYGFCRATDDLCDDESVPGEHRKHQLQATRQFVKTMYAEKKLTEAHYSQLPATCVPAFRAFATHLRHIVSEESVYELLDGYRWDLEKKSVKDEDDLAYYSACVASSVGEMCTRIIAHHGQCSLSSDTIQLARQMGLVLQYINIARDIVTDSQGLRRCYLPQNWLTQQEIQNLQLGQPRELSDCRLRQISLKLVSKAELMMKQAEVGIDDLPYCQGGLRAACQVYAGIGRCLKMSSGYPNRAYLKKKERIKIVLKSIYL

SEQ ID NO: 233 - protein, 563 aa, Rhizopus delemar
  REGION 1..563: note = misc_feature - Rd.CrtYB_I1BJX8, Rhizopus delemar
MLTYMEIHLYFTLPVVGLLLWILKPFHSSQDTFKYKFLLTIAFVTASVWDNYIVYHKAWSYCPTCVVVVIGFVPLEEYMFFIIMTVMTVAFANLMMRWHLASIFIKPNTPWIQSFFIRFVPILGLLTIAYKAWRLTVPGKPLFYGSCILWYACPVLALLWYGSGEFILRRSSSVLISIAIPTLYLCWVDILAIREGVWHISLRTSTGYFIVPHLPLEEFLFFTLINTVLVFATCTFDRVFIILQLTHRKVTMMNLIWAFALPDQALDHQLFHDLDQTQAILKRASQSFHAASAAFPAPIRQDLIVLYGFCRATDDLCDDDQVPIESRLQQLEVIRKFVQQVFSQKPTWLYIAALPASCQPTFRAFATHLAGILKEETVLELLDGYQWDLGCQAIRDEADLQYYSACVASSVGEMCTRVILYHEHAKQSSWLLDRARQMGLVLQYVNIARDIVTDSAQLGRCYLPQDWLTSDEIDQIAQGQARRLGEDRLKDLSIRLLSKADQLMKQAQRGLDGLPASSQGGVRAACSVYASIGTVLRQSKIYPTRAHLSSRQRAITVFKSIYL

SEQ ID NO: 234 - protein, 568 aa, Rhizopus microsporus
  REGION 1..568: note = misc_feature - Rm.CrtYB_A0A2G4SFV8, Rhizopus microsporus ATCC 52813
MLTYMEVHLYYTLPVLGILLWLLRPFDSKEDRFKYQFLACIAFFTASLWDNYIVYHKAWSYCPTCVVAVIGYVPLEEYMFFIIMTFMTISFTNLVMRWHLPSTFIRPQTPLIQTWSVRGVPIMALMIIAYKAWKLAVPAKPLFYGACILWYVCPVLAILWYGSGEYMLQRPLSVILSITLPTLYLCWVDKIAIAAGTWHISLRTSTGKFVSSDLPLEEFMFFVLINTVLVFATCTIDRAQAVTQLFNKHRHNTLDQLKTLLWAYLVPDQMLNPQVINDLSITWGILKSASKSFHTASAVFNYAVRQDLSVLYGFCRATDDLCDNESVPGKRRERQLQATRQFVKTLYAEKQLTEAHYSQLPVTCIPAYRAFATHLRYIVSEESVYELLDGYRWDLERRSVKDEDDLAYYSACVASSVGEMCTRIIAHHGQCSLSSDTIHRARQMGLVLQYINIARDIVTDSQDLHRCYLPQNWLTQQEIENLQLGQPRELSDRRLRQISLKLVSKAELMMKQAEAGIDDLPYCQGGLRAACQVYAGIGQCLKLSSGYPDRAYLEKRERIKIVLKSIYL

SEQ ID NO: 235 - protein, 602 aa, Sordaria macrospora
  REGION 1..602: note = misc_feature - Sm.CrtYB_D1Z4K7, Sordaria macrospora
MYDYAFVHLKFTIPLAVLLTAIAYPVLNRIHVIQTGCLIFIAFTAALPWDAYLIEQKVWSYPPEAIVGPRLLGIPFEELFFFVIQTYITALVYILFNKPVLHALHLNNQRNPPAWMRIAKVTGQLILVALSVWGWKAAQVNQKTTYLGLILVWACPFLLAIWTLAGRFILSLPWFVTVLPVVLPTFYLWAVDELALHRGTWSIGSGTKLEYCLFGKLDIEEATFFLVTNMLIVSGMAVFDQYLAVIYAFPTLFPKVNRYPTPLMLVQSRLVNTTKYDLERIEGLREAVERLRLKSRSFYLANSLFSGRLRIDLILLYSFCRLADDLVDDAKSRLEVLSWTAKLNHFLDQHYGDSDATEDPKQKAERIDAYIKEAFPPFAYQALHLLPTHILPPKPLYELIKGFEMDSQFTFHGSSDSTNLKFPIAHDKDLKTYAIRVAGTVGELCIALIIHHCLPDMSDSQKRRLESAACRMGIALQYVNIARDILVDAQIGRVYLPTSWLKEEGLTHKAVLDNPEGPEVIEKMRRRLLDNAFELYREARPEMQQIPSEARGPMIGAVENYMEIGRVLREKKVGQVFVRKEGRATVPKQRRLRTLLRALYEQ

SEQ ID NO: 236 - protein, 596 aa, Sporidiobolus pararoseus
  REGION 1..596: note = misc_feature - Sp.CrtYB_A0A0K0QV93, Sporidiobolus pararoseus
MGFDYWLVHAKWTIPPALVLWLFFRRLRTARDVYKTWFLITIAVLATIPWDSYLIRNRIWSYPQSSVVGPTLFAIPYEEVFFFFIQTYITSAVYALFTRPVVHAVLLPTKPEQGRTKRYFGTAIFLLVTALSITKIKEGGEGTYLALIVGWVAPFLALLWFITSTHILAMPRYSVILPILLPTLYLWECDARALQRGTWVIEQGTKLGLSYRGLEIEEAVFFLLTNLMIVFGLVACDYCLAVHDLRSYDKGYSSPFPPIQDFLPILANQPDTKQAQRIIDLRNAIDILEVHSKSFSTASMVFDGRLRLDLLSLYAWCRVCDDLIDNASSVPVASANITGIRTFLTYAYTRYDSTDQDSSTLPRPLTKVQLDTSLPLLSEKERGPFRLLTFLPITRGPLEELLTGFETDLSFIASALEEKEKKKNNDLPIKTDQDLMDYSSNVASSVADLCVQLVFAHSTPSSSSASSTSSDESKKRETLLAARKMGKALQLVNIARDVPADQKINRMYLPSRPIDSPIETLTEERRRLLKLAKEMAEESRVAIEGLPQEARGGIRAACDVYLSIGKAVEEALKDGRIEERARVGKFTRAKTAWRAL

SEQ ID NO: 237 - protein, 599 aa, Syncephalastrum racemosum
  REGION 1..599: note = misc_feature - Sr.CrtYB_A0A1X2H2H5, Syncephalastrum racemosum
MLTYMEVHLYYTLPMTGLLFLLLRPFHTAQDRLKYTFLCTIAMLTASPWDNYIVYHRAWWYCPSCVTAVIGYVPLEEYMFFVIMSLMTVAFTNLAMRWRLPSVFVKPDTSVAQSMCTRYVPIAFLLTIGVRAWNLAIPDTQLFYGACILWYVCPVLALLWFGAGEYMCRRWQTVLISILVPTAYLCWVDKVAIGAGTWHISLRTSTGKMVLPNLPLEEAMFFTLINTVLVFATCAIDRAQAILHLTKSSRLRSASQQQKQKESVVQQILDLTWAFCLSDQGLDNQQLKDIDVTWRILREASFSFYTASAVFPMNVRQDLGILYGFCRATDDLADDEQVSVEKRKDHLALARQFVQAMFRDKPNQPVMIDWEDFSNLPTCCLAAFRAFTRLRPALEVDAVEELLDGYAWDLQRRPILDDEALVRYSACVASSVGEMCTRVILYNHDSNIADTAVSRWTLERARDMGLVLQFTNIARDIVTDSQQLRRCYVPRTWLCAEEHDAICQGKARQHLGDGRLRELALQLVQVASDLEKRGRRGIHRLPSECQGGVRAACAVYSAIGGALQKAKGYPTRAHVSQAARAWIVLKNVYGFGQTTGIHP

SEQ ID NO: 238 - protein, 603 aa, Thamnidium elegans
  REGION 1..603: note = misc_feature - Te.CrtYB_A0A8H7SM09, Thamnidium elegans
MLTYMEVHLYFTLPVIGLLFYVLKPFHSKQDTLKYQFLTLMAVSTASVWDNYIVYHKAWYYCPTCVVAVIGYVPLEEYMFFVIQTLMTVAFANLVMRWHLPTFYIGVNVSISQSLVVKFVPIFGLLVVAYKAWYAAVPGKPLFYGSCILWYVCPVLALLWYGAGEYMLRRPLAVLTSIIVPTIYLCWVDQYAIGAGTWDISLKTSTGKMVVTNLPVEECLFFTMINIVLVFATCAIDRAQAIVHLYKSQSIHQRKQDQPFLQQMSDLVWAFCLPDQMLDNDILKDVAASWDILRTSSKSFYTASAVFASHDRLDLGILYGFCRATDDLCDDESVPVEKRKDQLKVTHQFVKELFSQKTNTPVIDWEFYAAQLPSSCVSAFKTFTRLRHILQVDAIEELLDGYKWDLERRPIVTQQDLYQYSACVASSVGEMCTRILLHNNDTGVHTNDRCILERAREMGLVLQLTNIARDIVTDSQELGRCYLPTDWLLKGEDELIATGKARQIGEPRLLNLSKKLIQDGERIMVTANKGIDQLPRNCQGGVRAACNVYAAIGTQIKQANERYATRAHLKSYGRSKIALASVYNVHTNTLKKARQGKIRSFIV

SEQ ID NO: 239 - protein, 595 aa, Trichophyton verrucosum
  REGION 1..595: note = misc_feature - Tv.CrtYB_D4D802, Trichophyton verrucosum
MGLDYLMVHVKYNIPPALLLTILYKPFFTRLEVYKIVFLCTIAVVWTTPWDSYLIRTRVWSYPADSVIGHTIFRIPLEEAFFFIIQTYNTSLIYILFNKRLILLPYLSGPIKPLTQGLFGTVALRTWRDFGILFFTGISVLGISCIRAGGEYMYLGLILSWISPILLLQWPLMYRFLLGLPPASLWVPIVLPTLYLWIVDTLALRRGTWVIESGTKVDIQLWDGLELEEALFFLVTNVMIVLGIAGMDNAIALFEYKAFVSTTAVGETPSIPRLLTLFFTRSRRYCDTNVLREMSQAVTLLKQKSQTMYLGSAMFEGQLRLDLVALYSFCRKADDLIDDAPNRATAQYWIKQCEKALELRFKLKGAALDNTAAYQQLTKSIPPQLHAAVHLLPASRLPKGPLSDLLKGFEIDMKFDSERGIFPIATEHDLEVYAYHVAGTIATLLLELVFRHHPVSISDSERLRVISAGEGMGRALQYTNIARDIVRDAEIGRVYIPSVWLAEQGLTPSMVVNQPRNPKLIPLRRRLLDKAEKCYRDTQEAISELPANVRAPVRATVTVYMDIGQVIRENEMKVWNGKLKVSRWRRFKGAWLAMS

SEQ ID NO: 240 - protein, 690 aa, Ustilago maydis
  REGION 1..690: note = misc_feature - Um.CrtYB_Q4P0S6, Ustilago maydis
MTSLDEPSQDWPACTLSYRHFHLLWTLPVCGVLLVIARPFLTKLDRAKLILLPIIAFVWTTPWDNLIVRNRAWSYHRHCIWFTIGYVPIEEYFYFIIQSLISTLWCTLITRWTLPNLYLTPSTPARRPLAPFAVATCVVCFVLGLQASVPATHTYYFGMISWWSSMPLALLLWGSIDFVRNMGVGAGFIPFAISVLGPTLYLWSSDIYALRRGTWHINQATSLNIFPIPDLPIEEMLFFLVTNVILVSACFTFDRCVAICRLSAPDHQPPLSPSYLPLGSLATYTKLWAAFVRSDRSSLPTSCASASVEPQDLSASLQVLRAASKSFNAASLLLPWDLRTDLGCLYAFCRVADDLVDDHAQELQAKAKNLDVVRDIVDAVYADSIVDRKQHLSTPIGERLRILLASAALSDKIKQDTRAAATSIAPLTQYIPKRLWYEMLQGYSADLRFQHQDARQRTRLQSMDDLVEYSQCVAGVVGEMCIRVILGRCGSTVPLDLEVDRKIALRASKTIEQSINVKPLDLNRTADVHTLLYEARRMGVSLQLVNIARDVVPDSVELRRCYLPADMFDKEEDDGMYEALLKGRVALPRHASKLVHGAEEQGVVRPQDVRKYALRLLEISQRLYDESYPSLRQIPNRPARAGLKAACSVYAAIGKRIQAQTEDEISSGRRARMSNLDRILRAISAVYFDV

SEQ ID NO: 241 - protein, 673 aa, Phaffia rhodozyma
  REGION 1..673: note = misc_feature - Xd.CrtYB mutant D52G, Phaffia rhodozyma (Xanthophyllomyces dendrorhous)
MTALAYYQIHLIYTLPILGLLGLLTSPILTKFDIYKISILVFIAFSATTPWGSWIIRNGAWTYPSAESGQGVFGTFLDVPYEEYAFFVIQTVITGLVYVLATRHLLPSLALPKTRSSALSLALKALIPLPIIYLFTAHPSPSPDPLVTDHYFYMRALSLLITPPTMLLAALSGEYAFDWKSGRAKSTIAAIMIPTVYLIWVDYVAVGQDSWSINDEKIVGWRLGGVLPIEEAMFFLLTNLMIVLGLSACDHTQALYLLHGRTIYGNKKMPSSFPLITPPVLSLFFSSRPYSSQPKRDLELAVKLLEEKSRSFFVASAGFPSEVRERLVGLYAFCRVTDDLIDSPEVSSNPHATIDMVSDFLTLLFGPPLHPSQPDKILSSPLLPPSHPSRPTGMYPLPPPPSLSPAELVQFLTERVPVQYHFAFRLLAKLQGLIPRYPLDELLRGYTTDLIFPLSTEAVQARKTPIETTADLLDYGLCVAGSVAELLVYVSWASAPSQVPATIEEREAVLVASREMGTALQLVNIARDIKGDATEGRFYLPLSFFGLRDESKLAIPTDWTEPRPQDFDKLLSLSPSSTLPSSNASESFRFEWKTYSLPLVAYAEDLAKHSYKGIDRLPTEVQAGMRAACASYLLIGREIKVVWKGDVGERRTVAGWRRVRKVLSVVMSGWEGQ

SEQ ID NO: 242 - protein, 673 aa, Phaffia rhodozyma
  REGION 1..673: note = misc_feature - Xd.CrtYB mutant E83K, Phaffia rhodozyma (Xanthophyllomyces dendrorhous)
MTALAYYQIHLIYTLPILGLLGLLTSPILTKFDIYKISILVFIAFSATTPWDSWIIRNGAWTYPSAESGQGVFGTFLDVPYEKYAFFVIQTVITGLVYVLATRHLLPSLALPKTRSSALSLALKALIPLPIIYLFTAHPSPSPDPLVTDHYFYMRALSLLITPPTMLLAALSGEYAFDWKSGRAKSTIAAIMIPTVYLIWVDYVAVGQDSWSINDEKIVGWRLGGVLPIEEAMFFLLTNLMIVLGLSACDHTQALYLLHGRTIYGNKKMPSSFPLITPPVLSLFFSSRPYSSQPKRDLELAVKLLEEKSRSFFVASAGFPSEVRERLVGLYAFCRVTDDLIDSPEVSSNPHATIDMVSDFLTLLFGPPLHPSQPDKILSSPLLPPSHPSRPTGMYPLPPPPSLSPAELVQFLTERVPVQYHFAFRLLAKLQGLIPRYPLDELLRGYTTDLIFPLSTEAVQARKTPIETTADLLDYGLCVAGSVAELLVYVSWASAPSQVPATIEEREAVLVASREMGTALQLVNIARDIKGDATEGRFYLPLSFFGLRDESKLAIPTDWTEPRPQDFDKLLSLSPSSTLPSSNASESFRFEWKTYSLPLVAYAEDLAKHSYKGIDRLPTEVQAGMRAACASYLLIGREIKVVWKGDVGERRTVAGWRRVRKVLSVVMSGWEGQ

SEQ ID NO: 243 - protein, 388 aa, Brevundimonas vesicularis
  REGION 1..388: note = misc_feature - Bv.CrtY_Q0H2D0, Brevundimonas vesicularis DC263
MQVDAFDTSAPDLLLVGGGLANGLLALRLSQVRPDLDVRIVEAAQTLGGVHTWSFFDSDLTQAQRDWIAPLVVHRWPGYSVRFPQFERALSTPYCSVTAERFAAVVEAALPGRIMLGAPVASVSPTEAVLADGRRLSAKAVIDGRGPTATPDLALGFQKFVGLEVRLAAPHGLTTPIVMDACVDQAGGYRFLYTLPFDDRTLLIEDTRYTDGDALDRAAFRQGVLDYAHAQGWEIETILREEDGVLPVALDGDIGAHLSRMGLTALSGLRAGLFHPTTGYSLPDAVRLADRLAQDFEPATVAEDIRRYAHDVWAGRGFYRLLNRMLFRAARPDERYKVLERFYRLPQPLVERFYAAGSTLADKARILSGKPPVPIGAALTCLVERGRA

SEQ ID NO: 244 - protein, 389 aa, Paracoccus sanguinis
  REGION 1..389: note = misc_feature - Psa.CrtY_A0A1H3ANX2, Paracoccus sanguinis
MTGAQDADLLIAGAGLSAGLIALAVRAARPDCRVLLVDAAPAPTGSHTWSFHLPDLPPAWLARLSPGLRARWDGQEVRFSDHARRLRAGYAALDDAGLARLVADSGAEVIRAAPVAQADAAGLVLADGRRLSAPCVIDARGARPSRHLVTGFQKFVGLEIETEAPHGVARPVIMDATVPQEDGYRFVYLLPFSPTRLLIEDTRYSDGAALDDAALADAIAAYARARGWAGREVRRERGVLPIALAHDAAGFWDEAAGGAVPVGLRAGLFHPVTGYSLPVAARVADAVAAASPLTTAAAMAAVRRLALTKAREDRFLRLLNRMLFRGCPPERRHRILSRFYRLPEGLIERFYAGRLTTLDRLRIVTGKPPIPIRDALPCLPEAPLFKESP

SEQ ID NO: 245 - protein, 434 aa, Erythrobacter longus
  REGION 1..434: note = misc_feature - El.CrtY_O06756, Erythrobacter longus OCH101
MSDSEIDSVPNDDSCDCAIVGGGLAGGLIALALQRARPEFRIRVIEAGRTIGGNHRWSWFDSDLSDAGRALLADFRQTDWEGGYEVRFPKYRRKLKTAYRSMASTDFHEGLLRALPEGSVILGRKAVGLDARGVDLAPSQYGPATRINARSVIDCRSFKPSAHLKGGWQVFLGRHMRLQEPHGVENPVIMDATVDQLAPHGNGGSYRFVYVLPLGSHDVFIEDTYYADDPLLDRNALSGRIDQYARANGWENGTPVHHEAGVLPVLTGGDFSAYQDEVRIPGVAIAGARGGFTHPLTSYTMCVAVENALAMAEQPDLSGEQLAAFFDSRARRHWSKTGYYRLLARFLFFAAKPEKRVKVFQRFYGLREGLIERFYAARSNTFDKVRVLWGEPPVAIHSAILAMFKSGPALKSEKSDRGVAQAALDEELQTEKRP

SEQ ID NO: 246 - protein, 395 aa, Rhizobium sp.
  REGION 1..395: note = misc_feature - Rsp.CrtY_WP_62583193.1, Rhizobium sp. Leaf321
MESGSHDLSLPIVLVGAGLASAVIAQRLSTVRDAPPIVILEATDTPFGEHTWSFHKADLQPSSLAWIAPLIAHEWDGQSVRFGSYERHLPSGYASLTSTSVSEAIERLPNVSLSKNTEVTALSAAQVTFANGSTQAASCVIDCRGYRPSASMVLGYQKFVGLEVELSEPHGLPHPVIMDATVDQLDGYRFVYLLPMSPTRVLIEDTRYSEGADLNDAVISEDIHTYATRQGWDITQVVRRENGVLPIVLAHDFERFWSELPADIPHAGLRAGLFHPTTGYSLPDAVHVADLIGENWPIGSAALPRAIRGHAARQHKKQGFYRLLNRMLFLAAEPDRRHLVLERFYKLPTPLIERFYAGRTTMTDILRILTGKPPVPIHRALACLSEAKLFENGSS

SEQ ID NO: 247 - protein, 388 aa, Devosia soli
  REGION 1..388: note = misc_feature - Ds.CrtY_A0A0F5L7L4, Devosia soli
MERPLVCAGAGLASALIAARLTRLHPEQKIILLEGSSEPFGEHTWSFHLADVNEADLEWLAPLIAHRWTGQAVRFQKLRRHLGSGYASLTSASVASAIAAMANVEVVAGAKVRDLAPESVTLADGRSIAAAAVIDTRGYTPSDALVLGYQKFVGLEVELEEPHGLSDPVIMDASVDQRDGYRFIYLLPFSPTRVLIEDTRYSDGEALDLQALAGDIARYAEAQGWSIRQVVREENGVLPIALAHDFERFWADKPVDIPQAGMRAGLFHPTTGYSLPEAVRIANLVAGNWPASSVTLARAIRDHARMRHREQGFYRLLNRMLFRAAAPERRHLVLQRFYKLPQPVIERFYAGRSTLGDIARILIGKPPVPIQRALACLREAPLLKAKKA

SEQ ID NO: 248 - protein, 395 aa, Caulobacter vibrioides
  REGION 1..395: note = misc_feature - Cv.CrtY_A0A0V2F4L4, Caulobacter vibrioides
MESASEALRADVVLVGGGLANSLIALRLKALRPGLRVLLLEREATIGGEHTWCHFETDVDAAIGGWLRPLIVHRWTGYEVRFPAHSRRLPTPYLAITSRRLHEVVSRVLGPDAWTGVAVVDVNPHQVTLADGRRIVAGAVIDGRGPRRSKRLALGWQKFVGQDLRLVAPHGLTAPIIMDATVPQTDGYRFVYVLPLDQHRLLIEDTRYSDGPGLDRASLGAAIADYARAQGWTIEAVEREEHGVLPIALGGDIDAYWREARPQVAEVGLRAALFQPTTGYSLPDAARLADKVAALPRITSASVRACVVTHSKATWRKRRFLRLLNRMLFRACAPEERYRVLERFYRLSVPLIQRFYAARLTWRDKARVLIGKPPVPIGAAMKCISESSVFGGQDA

SEQ ID NO: 249 - protein, 386 aa, Paracoccus sp.
  REGION 1..386: note = misc_feature - Psp.CrtY_P54974, Paracoccus sp. PAMC 22219
MTHDVLLAGAGLANGLIALALRAARPDLRVLLLDRAAGPSDGHTWSCHDPDLSPEWLARLKPLRRANWPDQEVRFPRHARRLVTGYGSLDGAALADAVVRSGATIRWNSDIATLDAQGATLSCGTRIDAGAVIDGRGAQPSRHLTVGFQKFVGVEIETDRPHGVPRPMIMDATVTQQDGYRFIYLLPFSPTRILIEDTRYSDGGDLDDDALAAASHDYARQQGWTGVEVRRERGILPIALAHDAAGFWENHATGPVPVGLRAGFFHPVTGYSLPYAAQVADVVAGLPGPLTTDALRAAIRDFAIARARRDRFLRLLNRMLFRGCAPDRRYTLLQRFYRMPQGLIERFYAGRLTVADQLRIVTGKPPIPLGTAIRCLPERPLLKENA

SEQ ID NO: 250 - protein, 401 aa, Robiginitalea biformata
  REGION 1..401: note = misc_feature - Rb.CrtY_A4CHD3, Robiginitalea biformata HTCC2501
MEHFDFIIIGGGASGLLLAEAMGRDTWFDGKRIALFEKQEQKGNDRTWCFWESGEGAFDELIETRWDRMRFRGTGIDRVLPLLPLTYKMLRGEDFYASYYQKIATCPNIHVIREAVNHVEERPDGAFLKTSRASYTTSRVFSSVPFSQPPLPGRPYPLIRQHFIGWRVRVPRPVFDPDTPTFMDFGIPQRGNTRFMYVLPDSPTEALLEYTLFSKDLLEPGEYEAAIEAYLREEYGVGEYEILEKERGSIPMTCNDFTARDSKHITHIGIAGGWARPSTGYTFWNSTQLVPRLVKALKSGKRLQMARRDRFWFYDLVLLEVLAGNNARGGEVFSALFRRVPPTRILRFLHQQTSLGEDLRIIAACPKRLFIRALWRSFLKSLGGAATPQNRAAPPWPGGFR

SEQ ID NO: 251 - protein, 394 aa, Aurantimonas sp.
  REGION 1..394: note = misc_feature - As.CrtY_Q1YGX9, Aurantimonas sp. SI85-9A1
MNGVERHDRPIDVAFVGGGLANGLAAYRLARARPELSIRVFEAGEAVGGNHTWSFHAGDLSAAEHDWLAPFVRYRWDAYDVRFPQRRRQIATGYRSVSSEQFRAVIDAALGDRIERHAPVRAVTPTTLTLEDGRELAAVCVIDGRGHAPSPHLMLGFQKFLGQEIELAAPHGLSRPIIMDATVAQEDGYRFVYVLPLTATTLLVEDTYYADGPALSEERLRTTIADYLATHGWTAKAVLREERGVLPIALDGDIEAFWADRRGVPATGLAAALFHPTTGYSLPDAVRLADRLAGLTDLSAGAVFAATRAHSVAAWRERGFFRLLNRLLFFAGVPSKRYEILQHFHRLPDTLIGRFYAAQLSRLDMVRIMTGRPPVPVGNALKVLARHRLTGELA

SEQ ID NO: 252 - protein, 382 aa, Paracoccus zeaxanthinifaciens
  REGION 1..382: note = misc_feature - Pz.CrtY_P94791, Paracoccus zeaxanthinifaciens R1534
MSHDLLIAGAGLSGALIALAVRDRRPDARIVMLDARSGPSDQHTWSCHDTDLSPEWLARLSPIRRGEWTDQEVAFPDHSRRLTTGYGSIEAGALIGLLQGVDLRWNTHVATLDDTGATLTDGSRIEAACVIDARGAVETPHLTVGFQKFVGVEIETDAPHGVERPMIMDATVPQMDGYRFIYLLPFSPTRILIEDTRYSDGGDLDDGALAQASLDYAARRGWTGQEMRRERGILPIALAHDAIGFWRDHAQGAVPVGLGAGLFHPVTGYSLPYAAQVADAIAARDLTTASARRAVRGWAIDRADRDRFLRLLNRMLFRGCPPDRRYRLLQRFYRLPQPLIERFYAGRLTLADRLRIVTGRPPIPLSQAVRCLPERPLLQERA

SEQ ID NO: 253 - protein, 397 aa, Devosia enhydra
  REGION 1..397: note = misc_feature - De.CrtY_A0A1K2HSZ0, Devosia enhydra
MPIPGQGPLVLVGAGLASALIAQRLAQSFPDQPILMLEAAAAPFGEHTWSFHEPDISRADWQWVGPLVAHRWPGQQVRFVAHARKLSTGYGSLTSQSVADAMARLANLTIRTLAPVRDIRPDGVTLSSGEAIPAACVIDASGHRQSPHMLLGFQKFVGLEIETTEPHGVDVPMIMDASVDQKDGYRFVYLLPFSPTRILIEDTRYADGEALDMQALAADISAYAEAKGWTLRTVVREEHGVLPIALAFDAERFWADAPKDIPQAGMRAGLFHPTTGYSLPEAVRIANLVAESWPAGSAALAERIRRHALKRAKDQSFYRFLNRMLFKAAVPDRRHLVLQRFYRLPQPLVERFYAGRTHWGDIARILIGKPPVPVSRALPCLLEAPQLNRSNKKTVTS

SEQ ID NO: 254 - protein, 382 aa, Pantoea agglomerans
  REGION 1..382: note = misc_feature - Pa.CrtY_Q47844, Pantoea agglomerans Eho10
MRPHYDLILVGAGLANGLIALRLQQQQPDMRILLIEAAPQAGGNHTWSFHDADLTESQHRWVAPLVVYHWPDYQVRFPTRRRKLNSGYFSVTSQRFAEVLQQKFGQHLWISRAVAEVHADAVRLNNGQVISASAVIDGRGYTPNSALNVGFQAFIGQEWRLSKPHGLSSPIIMDATVDQQNGYRFVYSLPLSATELLIEDTHYIDNATLEPERARQNIRDYAAQQDWQLQTLLREEQGALPITLTGDSTAFWQQQPLACSGLRAGLFHPTTGYSLPLAVALADRLSALDVFTSSSIHQAITHFAHERWQQQRFFRMLNRMLFLAGPADSRWRVMQRFYGLPEDLISRFYAGKLTLTDRLRILSGKPPVPVLAALQAIMTTHR

SEQ ID NO: 255 - protein, 388 aa, unidentified
  REGION 1..388: note = Eb.CrtY_Q2VJ84, Enterobacteriaceae Bacterium DC260
MRTQYDVILVGAGLANGLIALRLRQLQPQLKCLLLESDAHPAGNHTWSFHHSDLSAEQLRWLQPLITVRWSGYQVRFPALRRNLDGDYCSIASGDFARHLYAAMGDDLWTNTAVQQVKPTQVTLADGRELAAQVVIDGRGLQPTPHLQLGYQVFLGQEWQLAQPHGLQQPILMDATVDQQAGYRFVYTLPLSADRLLIEDTHYVNQPALAENTARQHIADYANQQGWTLSTLLREEHGILPITLSGNIDRFWQQQRGQACSGLRAGLFHATTGYSLPSAVALAELVAALLPTDALTLSQHIERFARQQWREQRFFRLLNRMLFLAGKPQQRWRVMQRFYRLDAGLISRFYAGQLRLRDKTRILCGKPPVPIGEALRALLNSVEPGKKK

SEQ ID NO: 256 - protein, 393 aa, Thermobifida fusca
  REGION 1..393: note = misc_feature - Tf.CrtY_Q47KA1, Thermobifida fusca YX
MERVDVAIVGAGAAGLSLAHRLDRVLPHRSVVLVETPEAALRSPERTWCFWEEETGEWDAAVCHRWHALAVQGPDGTTWRSRIHPLVYKMIRSRDFEALVRGSLQRVRTLHWTVTDIVDGPGEAVLRLRGADGAQRELAAGLVFDSRPLVGLPAEGVTLLQHFRGWFVRTDRPRFDSSAAVLMDLRVPQPERGVAFGYLLPFDARTALVEYTEFTREVCSDAAYDAALRDYTENLLHLGVFTVTGVEQGVIPMTSAHLPERVGHRVFRIGTAGGATRPATGYTFAGVQRQTRRIAAALVAGRFPVGYSAYPARHRFFDAVLLRGLDEGVVDGAEFFATLFRSNPLPRLLRFLDGSSRPWEELAIGLGTPVADMSTALWRQVRAGRRVASSLPR

SEQ ID NO: 257 - protein, 395 aa, Parvularcula bermudensis
  REGION 1..395: note = misc_feature - Pb.CrtY_E0THV9, Parvularcula bermudensis HTCC2503
MTSYDVLFAGAGLASSLTAYRLARRTPSLHIGMVEQGAAVGGNHTWCFHETDIAPAILEGLKPFILKRWPGQSVRFPGHDRDLPTPYCAMTSQRLAAVLSGVPSIDVITGAQVTKATADRLILADGRELSAPCVFDGRGPGRVRHMRLGFQKFLGLEVTLAAPHGLNQPIIMDATVPQTDGYRFVYVLPFSETELLIEDTYYADGFDLSESHLEAEIIHYAARKGWTIATISRREQGVLPIVLEGDFEAFWAERRTGAVPIGLAAGLFHPVTGYSVPDAATLADRLGRDTPLTSDRVRPIVEDYARDRWSAHGFYRLLNRLLFRAAAPDQRYIVLERFYRLPIPLIERFYAGETNTGDKLRVLAGKPPVPLMRAISALRPQPLGHSAGMREGILG

SEQ ID NO: 258 - protein, 391 aa, Cronobacter sakazakii
  REGION 1..391: note = misc_feature - Cs.CrtY_A7MP98, Cronobacter sakazakii ATCC BAA-894
MNTQWDLILAGGGLANGLIALRLRVRQPRLNVLLLEASDAPGGNHTWSFHGADLTAEQHAWLTPLVAHRWDGYEVRFPALTRTLDGGYYSVTSERFAHVLQEAVGERLWRNAPVASLTPQSVTLTDGRTLHARAVIDGRGWQASSHLTAGQQAFLGQQWRLAAPHGLTRPVLMDATVAQQGGYRFVYTLPLTPDTLLIEDTHYIDSATLDHDRARENIASYARLQGWQLATLEREERGNLPITLTGAPQAFWREAQGVARAGLRAGLFHATTGYSLPHAATLADLIAAQPPVSSAALYGLTAGYAQRQWRRQRFFRLLNRMLFLAGQPDRRWQVMQRFYGLNAGLIARFYAGRLTPMDMARLLTGKPPVPVGEAMQAVLKQTPRLRAFHHD

SEQ ID NO: 259 - protein, 390 aa, unidentified
  REGION 1..390: note = CPe.CrtY_X5MCP7, Candidatus Phaeomarinobacter ectocarpi
MPNATYDLLLAGAGLANALIVRALKASHPDLKIGIVDRNPHPDPSHTWSFHTSDISDDARAWLDPLIAHEWHGQDVRFPGHARALKTGYASISEEKLRHQLAQDMEATPILEGDVTSLDEEGVTLEDGRHIDAHAVIDGRGQKANTSLAIRFQKFVGQELELEEPHVLTRPVIMDATVPQEDGYRFFYLLPHTPTSLLVEDTRYSDGAELNDDVFRDAIENYATGKGWKIKKVLREERGVLPIALGGNFDAFWEGTPKGTGRSGLAAALFHPVTGYSLPDNVRLAQAIAALPHITSQTVAQTTRAHAKAAWDKRGFYRLLNRMLFDAARPQDRYKVLERFYRLPEPLIARFYAGQTTLADKARILTGKPPVPILAAMRCLKEPKSGVSQP

SEQ ID NO: 260 - protein, 390 aa, Aureimonas
  REGION 1..390: note = misc_feature - Asp.CrtY_A0A0Q6E1S9, Aureimonas
MPERFDLVLVGGGLANGLIAHRLAVLRPDIGVLVLEAEAAPGGNHTWSFHAGDLSEAEAAWLAPFVVHRWPAYDVRFPRRLRRLGTGYASVTSERFAEVLQAGRPGLVRCGMRVAELRPDGVTLSDGTQIAAAAVIDGRGPRPSARLSIGYQKFLGRELRLSAAHGLSAPVVMDATVAQDDGYRFVYTLPFDERRLLVEDTYYADGPGLDGADLALRIAAYVAERGWAAAEVLREEEGILPIALGGDIEGFWADRAGVAASGLSAALFHPTTGYSLPDAVRLADLVAGLPDLSAPALFAATRDHSIRLWRERRFFRLLNRMLFLAGASERRYEILQHFHRLPEDLIARFYAARLSLGDKARILAGKPPVPVGRALRALAFPRSLSPEDAA

SEQ ID NO: 261 - protein, 385 aa, Xanthobacter autotrophicus
  REGION 1..385: note = misc_feature - Xa.CrtY_Q93CI8, Xanthobacter autotrophicus Py2
MDIVFVGAGLANCLMAARLAAQRPGLHMLLLEAGESVGGNHSWSCHDSDLTAAQRAFLAPFQSYLWAGHGVHFPAFSRTLKGGYATISSERMAEVMNERLCAAIRTNARVAHVAPDHVVLEGGERIDARAVVDGRGPLASRHLDLGYQTFLGQELRMSRPHGLTRPIIMDARVEQLGGYRFVYVLPLDDDTLLVEDTYYADGPDLPADALRGRISAYAAAQGWAVDYVVREEDGILPIALGGDINAFLAETPSGVAPAGLRAGLFHPTTGYSLPDAMALADSVSALADLSGPALSAAVRSHAAAAWNGRGFFRLLNRMLFRAADPERRYAILQRFYGLSEDLIARFYADRLTLADKARILSGRPPVSVFRALSCLVETKAAPGSP

SEQ ID NO: 262 - protein, 382 aa, Paracoccus sphaerophysae
  REGION 1..382: note = misc_feature - Psph.CrtY_A0A099F1A2, Paracoccus sphaerophysae
MSGVLIVGAGLAGSLAALALRAARPGLPVTLLDAAPGPSDRHTWSFHHPDLPPVWRDRLAPGLRASWSLQEVRFPDHGRVLEAGYASLDGRGLAQLVAGSGATTLWSRTAAEVLAHAVTLSGGTRLTADAVLDARGLRPSRHLVAGYQKFVGVEYEMDAPHGVICPLIMDAKVPQLDGYRFVYLLPFSPTRLLIEDTRYSDGAGLAEPDLIAAITDYAAVHGWQGREVRRERGVLPIALAHDAAGFWAAQDGAVPIGLRAGLFHPVTGYSLPVAVQVADVIAAADPTTAAIRAAVRDFALRKAHGDRFLRLLNRMLFRGCAPDERRRVLSRFYRLPEPLIERFYAGRLTTLDKLRIVTGKPPIPIRDALPCLPESPLLKDAR

SEQ ID NO: 263 - protein, 392 aa, Brevundimonas sp.
  REGION 1..392: note = misc_feature - Bs.CrtY_Q4W8B7, Brevundimonas sp. SD212
MQAAAPDSSAPDLLLLGGGLANGLLALRLSQVRPELDVRIVEAADRLGGIHTWSFFEADLTPAQRAWIAPLIACRWPGYSVRFPAFERRLVTGYCSVTAERFAEAVTQALAGRIVTGAAVVSAGPTEAVLADGHRLTARAVIDGRGPTAAPDLALGFQKFVGLEVRLTAPHGLKEPIVMDACVDQSGGYRFLYVLPFDDRTLLIEDTRYTDGDDLDHDLFRTGVRDYAAQRGWVIETVLREEEGVLPVALDGDIAAHLKRLGPTALSGLRAGLFHPTTGYSLPDAVRLADHLAERIEAAPDGPALAQVIRRHARDVWAQRGFYRLLNRMLFRAARPDQRYRVLERFYRLPQPLIERFYAGETTLADKARILSGKPPVPIGAALTCLVERGRA

SEQ ID NO: 264 - protein, 395 aa, Caulobacter henricii
  REGION 1..395: note = misc_feature - Ch.CrtY_A0A0P0P0Q7, Caulobacter henricii
MATQSSNTRADVVLVGGGLANGLIALRLRTLRPDLRVILLEREKRIGGEHTWCHFGTDVSPGISGWLRPLIVHRWSDYEVRFPGHSRALKTPYLAITSQRLHEAVSAALGQDAWLSATASEVSPHQVTLTDGRRITAAAVVDGRGPRRSRSLALGWQKFLGQEVKLTSPHGLTRPIIMDATVSQLDGYRFLYVLPLDASRLLIEDTRYSDGFDLDRDALRADILAYANQQGWTVEKIVREEHGVLPIALAGDIDAYWREARAQVAEVGLRAALFQPTTGYSLPDAARLADQIAALPHITSAHVRACAEAQSKTAWRKRRYFRLLNRMLFRACEPDQRFKVLERFYRLRLPLIQRFYAARLTLWDKARVLIGKPPVPVSRALNYLSEHSVIEDRPA

SEQ ID NO: 265 - protein, 382 aa, Pantoea ananatis
  REGION 1..382: note = misc_feature - Pa.CrtY_P21687, Pantoea ananatis ATCC 19321
MQPHYDLILVGAGLANGLIALRLQQQQPDMRILLIDAAPQAGGNHTWSFHHDDLTESQHRWIAPLVVHHWPDYQVRFPTRRRKLNSGYFCITSQRFAEVLQRQFGPHLWMDTAVAEVNAESVRLKKGQVIGARAVIDGRGYAANSALSVGFQAFIGQEWRLSHPHGLSSPIIMDATVDQQNGYRFVYSLPLSPTRLLIEDTHYIDNATLDPECARQNICDYAAQQGWQLQTLLREEQGALPITLSGNADAFWQQRPLACSGLRAGLFHPTTGYSLPLAVAVADRLSALDVFTSASIHHAITHFARERWQQQGFFRMLNRMLFLAGPADSRWRVMQRFYGLPEDLIARFYAGKLTLTDRLRILSGKPPVPVLAALQAIMTTHR

SEQ ID NO: 266 - protein, 394 aa, Bradyrhizobium sp.
  REGION 1..394: note = misc_feature - Bo.CrtY_Q9KIX3, Bradyrhizobium sp. ORS278
MSRDADVIVIGGGLAGCLIALRLTDARPDLRVVIIEGSASIAGNHTWSFFGTDISSDQHAWLGRLVGHRWPGYEVRFAEHAIRLSTAYLSMTSTRLRAEVEQRFPERILRDATAISATADHVVLEGGRTLRAPCVIDARGGRPVPGLALGFQKFLGLEVRLAAPHGLDVPIVMDATVAQSDGYRFVYTLPLDPQRLLIEDTYYSDGGELPEQVLHQRIARYALAKGWQIAEIIRAEQGVLPVILAGDPSGLVSKPDSPPRVGLAALLVHPTTGYSLPDAVRVADLLTARLAQRGALSSADARETIDGYGRTIWRRRGYYRFLNRMLFKAAEPSERHRILARFYGLDQALIERFYAARIQPQDKLRVFMHMLMKPPIPISSALACLPEASAFKTP

SEQ ID NO: 267 - protein, 232 aa, unidentified
  REGION 1..232: note = Umv49E08.CrtY_A4GIB9, Uncultured Marine Bacterium HF10_49E08
MIYLEFLILFIGIPLFAISFWAYKKQCLDKKNITGIGLMCVIALVYTTPWDNYLIMENIWSYPAGVVFGTIGYVPIEEYGFMLMQTMLAGVLWSLVSSKINVSGLTLSGKGILVSLIPGFVGSYCLSHETGTYAGLILVWAFPPLALQWGLGARALLTSAKMWMPLWVGFTLYLCLADAFAISDGIWTITSNTRSGIEIMGILPIEEALFFALTNLFVLQGLCLWQTWRNKG

SEQ ID NO: 268 - protein, 360 aa, unidentified
  REGION 1..360: note = Umb29C11.CrtY_A4GI69, Uncultured Marine Bacterium HF10_29C11
MKTQILGDGVAAMMLASHADELPNHEISIVHPTGAPITRDHMLGFWKMDGLEMAVESSRTSWSKWAIITDGNKSVMHSDKHAYHIMHKANYLHNCREKAEQHGVKFIEESEMITSKSAQTFDSRPPRASKNAMLQHFLGQEVEVDKPVFDPSTAILMDFRVDQSEGMHFIYLLPYSPTQALVESTLFTTTVLERQFYVDAINEHLRDHYGASITNIIHEEQGVIPMGTLSPHDDTIPGIGANAGAIRPASGYTFVFIHQQIQRAIKASKQGKPLRFKRPHKAVDVWMDAVLLTVLKNWPQQGPKLFGRMASSLSGDEFVRFMSGQSNWRLRFKVIMAMPKLPFIRGVSKHIFRRSEKVVA

SEQ ID NO: 269 - protein, 417 aa, Synechococcus sp.
  REGION 1..417: note = misc_feature - Sk.CrtY_A0A076HH15, Synechococcus sp. KORDI-52
MAEPVDVLVLGGGPAALCVASELNQRGVSVAGIASDPVDAPWPNTYGIWADELKAVGLEQLLEHRWSDTVSFFGEGGSTAQDRSHAHGIDYGLFDRAVLQRHWLERADGVVWHQDTAERVELKGSTTLVSCASGTTMQARLVIDASGSRTHHIRRPDQGPVAGQAAYGVVGRFSKPPIEPDRFVLMDYRCDHLSETQRQEPPTFLYAMDLGDGVFFVEETSLALAPAVPYDVLQQRLQQRLDQRGVEITEVIHEEFCLFPMNLPLPDRRQPLLAFGGAASMVHPASGYMVGSLLRRGPDLAQALAEAITNPSLGSAALAQRGWQALWPLELVLRHQLYQFGLGRLMGFNEALLRTHFATFFALPRDEWFGFLTNTLPLPRLMSVMLRLFALAPWELRRGLVLGAPPSQSPTWTQSNG

SEQ ID NO: 270 - protein, 428 aa, Sinorhizobium meliloti
  REGION 1..428: note = misc_feature - Sm.CrtY_WP_50578984.1, Sinorhizobium meliloti
MVDPLAPETASTGKTGTAAIAGRDLARAGDRERLESESIDPSLPVVLVGAGLASAIIAQRLSTVRDAPQIVILEGTDTPFGENTWSFHKADLQPSSLGWIAALIAHQWDGQSVRFGGYERHLPSGYASLTSASVRDAIERMPNVTLNKNTEVTALSTAQVTFANGSTQAASCVIDCRGYRPSSSMVLGYQKFVGLEAELSEPHGLPHPVIMDATVDQLDGYRFVYLLPLSPTRVLIEDTRYSEGADLDHAVISDDIHAYATRQGWDITQVVRTEDGVLPIVLAHDFERFWSELPEDIAHAGLRAGLFHPTTGYSLPDAVHVAEIIAENWPICSAALAHAIRGHAARQHKKQGFYRLLNRMLFLAAQPDRRHLVLERFYNLPTPLIERFYAGRTTTTDILRILTGKPPVPIHRALACLNEAHLFENGSS

SEQ ID NO: 271 - protein, 386 aa, Enterobacter agglomerans
  REGION 1..386: note = misc_feature - Eh.CrtY_L0BIU3, Enterobacter agglomerans
MPRYDLILVGAGLANGLIALRLRQQRPSLRILLIDAEREPGANHTWSFHAEDLTETQHRWIAPLVVHHWPGYEVRFPQRSRSLNSGYFCVTSERFVQVIRDRFAPDLLLNTRVAGIASRTVTLDDGRVLESDAVIDGRGYQPDAALCMGFQSFVGQEWQLSEPHGLTAPIIMDATVDQQAGYRFVYSLPFSADTLLIEDTHYIDNATLEGDRARQNIRAYAAQQGWRLDRLLREEQGALPITLTGDVAAFWQKHDLPCSGLRAGLFHPTTGYSLPLAVALADRLAQMQTFTSETLHATIQQFASQAWQQQRFFRMLNRMLFLAGPADQRWQVMQRFYGLPEGLIARFYAGKLTLPDRLRILSGKPPVPVLAALQAIMTPHRQQAMQ

SEQ ID NO: 272 - protein, 236 aa, Algoriphagus sp.
  REGION 1..236: note = misc_feature - Ak.CrtY_Q15I92, Algoriphagus sp. KK10020C
MGNYLYLAVNLFAISFPLIRSFEPKINFARKWYALFPALVITAIFFLVWDHWFTVIGIWEFNPNYLLGIYLFKLPIEEWLFFFTVPFACVFIYEVLIYFFPKDIFRPVGRPFVLVLIPILLLLAFLNLDKLYTSVNFFVGAFALAIHWLIFKDKFLGRFIFAYLVHLIPFIICNGILTGGLTEEPVVIYNNAENLGIRIWTVPIEDTIYSMTLLLMNVSLFEYFRSRKTIQQTKSY

SEQ ID NO: 273 - protein, 389 aa, Pantoea sp.
  REGION 1..389: note = misc_feature - Pa.CrtY_E6WK12, Pantoea sp. (strain At-9b)
MPANSHYDVILVGAGLANGLIALRLRQQHPSLRCLLLESSAQADSNHTWSFHQGDLRAEQHRWLAPLVTAHWPGYQVRFPALQRNLPDAYFSISSANFARQLYATLGDDLRTGCAVHLITPTSVTLTDGRQFQAQVVIDGRGMRYSPHLRLAYQVFVGQEWQLAKPHGLTQPLLMDATVPQQQGYRFVYTLPLSADRLLIEDTHYMDTPLLDDNETRKRISDYARQQGWQPSLLMREERGALPIPLSGDINAFWQQQRGQPCSGLRAGLFHATTGYSLPMAVTLAELIAERLPCDAAVLSQHIEAHARRHWHQQRFFRLLNRMLFLAAQPEQRWQVMQRFYQLDAALIARFYAGQLTLADKARILCGKPPVPPGAALRALLTTQTGKHP

SEQ ID NO: 274 - protein, 386 aa, Erwinia uredovora
  REGION 1..386: note = misc_feature - Eu.CrtY_P54974, Erwinia uredovora
MTHDVLLAGAGLANGLIALALRAARPDLRVLLLDHAAGPSDGHTWSCHDPDLSPDWLARLKPLRRANWPDQEVRFPRHARRLATGYGSLDGAALADAVVRSGAEIRWDSDIALLDAQGATLSCGTRIEAGAVLDGRGAQPSRHLTVGFQKFVGVEIETDRPHGVPRPMIMDATVTQQDGYRFIYLLPFSPTRILIEDTRYSDGGDLDDDALAAASHDYARQQGWTGAEVRRERGILPIALAHDAAGFWADHAAGPVPVGLRAGFFHPVTGYSLPYAAQVADVVAGLSGPPGTDALRGAIRDYAIDRARRDRFLRLLNRMLFRGCAPDRRYTLLQRFYRMPHGLIERFYAGRLSVADQLRIVTGKPPIPLGTAIRCLPERPLLKENA

SEQ ID NO: 275 - protein, 498 aa, Capsicum annuum
  REGION 1..498: note = misc_feature - Ca.CrtY_Q43415, Capsicum annuum
MDTLLRTPNNLEFLHGFGVKVSAFSSVKSQKFGAKKFCEGLGSRSVCVKASSSALLELVPETKKENLDFELPMYDPSKGVVVDLAVVGGGPAGLAVAQQVSEAGLSVCSIDPNPKLIWPNNYGVWVDEFEAMDLLDCLDATWSGAAVYIDDKTTKDLNRPYGRVNRKQLKSKMMQKCILNGVKFHQAKVIKVIHEESKSMLICNDGITIQATVVLDATGFSRSLVQYDKPYNPGYQVAYGILAEVEEHPFDVNKMVFMDWRDSHLKNNVELKERNSRIPTFLYAMPFSSNRIFLEETSLVARPGLGMDDIQERMVARLSHLGIKVKSIEEDEHCVIPMGGPLPVLPQRVVGIGGTAGMVHPSTGYMVARTLAAAPVVANAIIQYLSSERSHSGDELSAAVWKDLWPIERRRQREFFCFGMDILLKLDLPATRRFFDAFFDLEPRYWHGFLSSRLFLPELIVFGLSLFSHASNTSRLEIMTKGTLPLVHMINNLLQDKE

SEQ ID NO: 276 - protein, 411 aa, Cellulophaga sp.
  REGION 1..411: note = misc_feature - Cm.CrtY_Q55276, Cellulophaga sp. MED134
MFDALVIGSGPAGLAIAAELAQRGLKVQGLSPVDPFHPWENTYGIWGPELDSLGLEHLFGHRWSNCVSYFGEAPVQHQYNYGLFDRAQLQQHWLRQCEQGGLQWQLGKAAAIAHDSHHSCVTTAAGQELQARLVVDTTGHQAAFIQRPHSDAIAYQAAYGIIGQFSQPPIEPHQFVLMDYRSDHLSPEERQLPPTFLYAMDLGNDVYFVEETSLAACPAIPYDRLKQRLYQRLATRGVTVQVIQHEEYCLFPMNLPLPDLTQSVVGFGGAASMVHPASGYMVGALLRRAPDLANAIAAGLNASSSLTTAELATQAWRGLWPTEKIRKHYIYQFGLEKLMRFSEAQLNHHFQTFFGLPKEQWYGFLTNTLSLPELIQAMLRLFAQAPNDVRWGLMEQQGRELQLFWQAIAAR

SEQ ID NO: 277 - protein, 343 aa, unidentified
  REGION 1..343: note = Cp.CrtY_Q4FPE3, Candidatus Pelagibacter ubique HTCC1062
MSLAYELDLHNKLKDATLAIIEPRLEYKRDKTWSYWKTIDHNFQDCVQKSWNNFSINIPGKTKFLECNGLPYEAIDSGLFYEKINTRLKKNKNISFYKNTNEIDLNSAFIFNSVPLLDSKEDKLWQHFKGIEIETKKNVFDDEIFNLMDFDCDQRDSVHFFYTLPYTKNKALIETTWLSKIKNDSLKDYDEQLKNYIRNHLNIRDYKVTYSEVGAIPLFHPTISNERNKMNIGTAGGMTRLSTGYTFLNIQEHSKYIRKNIHTIKESKRYEINKKYKFLDKIFLKVLNYHPEKMANIFFDMFNNSPETIIKFLSNKSTLLQDLFIILKMPKWLFIKIFFKNDK

SEQ ID NO: 278 - protein, 513 aa, Chlorobium phaeobacteroides
  REGION 1..513: note = misc_feature - Cp.CrtY_A1BCY2, Chlorobium phaeobacteroides DSM 266
MIADKVQTLYPRVSEYFGKIRNGNRYLEHILEVDSFWKNFEHSPCRKVVLQGSEMPADACVSDEYDLVYAGGSLGLLHASVMASKYARKVLVFDRHMSGKPSRDWNISWNELEKLEQVGLFSYEEINASIACRYKTGWAEFYVENGRKKRLYIDNVLDCAVESDQLLSIARQKILADECNRIVDKMTFMRCFQFPESVVIEVENNMHERVYFRAKVFVDAMGVHSPVAMQLNDGASFTHLCPTVGTVSSGLENVDPDIGEILVSTEPADTSSGRGRQLIWEGFPAGGDRYTTYLFFYDSRSSDNDKSLLGLFETYFLKLPSYKKPGKDFVVHRPVYGVIPAYYHDGFERTRQVADNNILMLGDAAALGSPLTFCGFGSFVRNLKSLTEGLEKALACNSFEKKNLEQISAYEPNVAAMANLMKFMCYNKYTDHPNFVNELMNEVMIVLDGLPPRYREAMFKDTLELSDLLLIGLNVAWKYPGVLMATATKLGLEGSLGMMKNMIGWAMSSKASN

SEQ ID NO: 279 - protein, 512 aa, Chlorobaculum tepidum
  REGION 1..512: note = misc_feature - Ct.CrtY_Q8KF75, Chlorobaculum tepidum TLS
MLEQIRNTHPLVYQNFSALPDGEKHLRSILAIDRYWEKLDLPVPDVILAGSRLPVDACVEEACDILYAGGTLGLLHAAVMSKKYGRKVLVIDRAEPGRTTRDWNISRGELLRLADTGVFTSEELDSTIVRMYKTGWVEFHAPAERRKRLYMDEVLDCAVDADRLLGMACKKVLAGGGSKVLGHTSFVCCYQFPDHLVVQVEELSGKPRYFRTQVLVDAMGIVSPVAMQLNRGRPQTHVCPTVGTIASGFENADFEVGEILASTEDAEVSGKRGRQLIWEGFPAKGDEYITYLFFYDKVDSPNDKSLLGLFEAYFRKLPEYKKPGPNFTIHRPVFGIIPAYFHDGAGCTRVVSGERIALLGDAASLASPLTFCGFGSVVRNLDRMTSGLDRAMREGRLGAAELANISAYEPNVASMATLMKYMCYDPETDEPGFVNEMMNEVMIVLDELPQRYRQAMFRDEMKVEELVTVMLKVAWRYPKILKATWNKLGVGGSVGFVKNLAGWAISQNEKRG

SEQ ID NO: 280 - protein, 392 aa, Fulvimarina pelagi
  REGION 1..392: note = misc_feature - Fp.CrtY_Q0G4C0, Fulvimarina pelagi HTCC2506
MTSSAKQKVDIALVGGGLANGLIAWRLAELRPDLSIVVLEAGEAPGGNHTWSFHEHDLTPAAHRWIAPFVAHRWTTNEVQFPDRHRHLSTGYLSASSDLFRERLTTRLGLRIRTGCPAVSVTARKVRLENGEVIEAGSVIDGRGYRSSEHLTLGFQKFLGQEIEFEAPHGVARPVIMDATVPQADGYRFVYLLPMTPTRLLVEDTYYADGDALDRGTIRRNIAAYRAAKGWPAGKVVREEDGVLPIALAGDIEAFWEEKQGVPSSGLNAALFHPTTGYSLPDAVYLADLIAGLPDYSAATLYAATRRHSVATWKRRGFFRMLNRLLYLAGDPLKRYVILQHFYRLPEPLVSRFYAARLTRGDKVRILTGKPPVSVISALKVLSPSSVEGAPA

SEQ ID NO: 281 - protein, 269 aa, Halorubrum lacusprofundi
  REGION 1..269: note = misc_feature - Hl.CrtY_B9LUN5, Halorubrum lacusprofundi ATCC 49239
MLPTLTYLQFHLVFSLPVIAALWYVAPRYGAVRQRRATIAIAILVAIAYTYTTPWISYMIRRGAWSYADGAVLVRALSIPLGEYLFFTVQTVVTAFALHRIGFDPTFREGDFDRGPRIAGVVAGVAMILGGLWLVTIGPSYLYLGGLIAWVGPVVAIQWAVGGGYLIRTPRTWIAATLIPAAYFWIADRIAIEMRTWQLSPDLTTGVAVLGLPIEELLFFVSAGVMTVNGLVLFEWVLDWNDRRERPTESLADALEPEPEPESAEPVDD

SEQ ID NO: 282 - protein, 512 aa, Ostreococcus lucimarinus
  REGION 1..512: note = misc_feature - Ol.CrtY_A4RQU2, Ostreococcus lucimarinus CCE9901
MRRGDATATATATAAFAKRREGASDARGETYDVAVAGGTLGVLVAAALQRRGARVVVVERGELRGREQEWNVSREELERLVRVGAITTEDADACTRIEFNPIRCGFHGGGNGNGNAGDLVTRDILNTGVSPAALVEACRKRFEEAGGAVLERASLRGVEVYDDCAVLDVDGVPVRARLVLDCMGFQSPIVRQIRDDRKPDGVCVVVGSCAEADAFDNSSADLIRTVTDIEEDYRGQYFWEAFPASTGPRDRTTYMFTYMDADESRPSIASMLDDYWDLMPLYQGLDSIEDAKLKRVLFGLFPTYRDSPLKTEFDRVLAIGDASGIQSPLSFGGLAAILRHISRITGACEEALDADCLDREALRTVNAYQPALSAAWLFQKCMSVEVGSRPKRDFINRLMRINFNVMSRLGEDVLRPFLQDVVTFKGLGKTLVSMTLTEPLFVPEILMNAGVGPILDWFKHFAALGAYDFLASPAAALADTVKDFSAVSPRRRFIIRRQCEAIIYGAGRDVIP

SEQ ID NO: 283 - protein, 386 aa, Paracoccus haeundaensis
  REGION 1..386: note = misc_feature - Ph.CrtY_Q24K62, Paracoccus haeundaensis
MTHDVLLAGAGLANGLIALALRAARPDLRVLLLDHAAGPSDGHTWSCHDPDLSPHWLARLKPLRRANWPDQEVRFPRHARRLATGYGSLDGAALADAVARSGAEIRWNSDIALLDEQGATLSCGTRIEAGAVLDGRGAQPSRHLTVGFQKFVGVEIETDCPHGVPRPMIMDATVTQQDGYRFIYLLPFSPTRILIEDTRYSDGGNLDDDALAAASHDYARQQGWTGAEVRRERGILPIALAHDAAGFWADHAEGPVPVGLRAGFFHPVTGYSLPYAAQVADVVAGLSGPPGTDALRGAIRDYAIDRARRDRFLRLLNRMLFRGCAPDRRYTLLQRFYRMPHGLIERFYAGRLSVADQLRIVTGKPPIPLGTAIRCLPERPLLKENA

SEQ ID NO: 284 - protein, 390 aa, Parvularcula oceani
  REGION 1..390: note = misc_feature - Po.CrtY_WP_31553302.1, Parvularcula oceani
MADAYDLLFAGGGLSSGLTAYRVAQARSDLSIAIVEAAPRLGGNHTWSFHATDVAPGTLRWLTPFIVHDWPAQRVAFPRRERRIETPYRSVTSERLHEVVSAVPNISLLTGKPVKAVRRDGLTLEDGAVLSGPVLDGRGARSSPHLSLGFQKFLGQELRTARPHGVETPLIMDARVPQTDGYRFVYVLPLGPDILHVEDTYYADGDDLPREALREEIMAYAHRQGWEVAEILREEDGVLPIVLEGDIDGFWDAHRAGPAPLGLRAGLFHPVTGYSLPSAARLADEIAEACGSAIPAPGALFELVEDHARRQWEAQGFYRLLNRMLFRAAKPDLRYVVLQRFYGLPRALIERFYAGETTAADKARILAGKPPVPLGAALRSLRPQALLGQG

SEQ ID NO: 285 - protein, 529 aa, Synechococcus elongatus
  REGION 1..529: note = misc_feature - Se.CrtY_Q31QI5, Synechococcus elongatus PCC 7942j
MSGSALRSVLNQLPARVAQGLHQSDAFWRSLRWGQLPTPQWVSESADPLGSCDLDVLVAGGTLGLLPALGLQRQGWRVGLWERGMVQGRDQEWNISRHELAVLVELELLTAEELEAAIATEFPAARIAFPGSAELWVEGILNIGISPRRLIATLLQHFREQGGQVFEQTAIDRVSVHPDGVVIGAGSQRWSGRLLLDALGHFSPIARQARAGAKPDAICLVVGGCAQGFPASDRGDLFVSRSPIQQQRQSFWEAFPAAEGRTAYLFTYIDAQPARPSLRSLLEEYLAALPEYQGVDLQQLQWKRLLWGILPSYRQSPLQSPWNRILAIGDSSSAQSPLSFGGFGALLRHLERLCRGIHEALLADCLQAADLALLQPHQPNLAVTWLFQQSMTVPLDRSLPPERINELLGRIFAVMESLGPQTLRPFLQDVVQFQPLAKTLLMTTLLHPVLVAKLLPQLGLQPLLQWLPQFLQLSLYRSLAIAGEPTDWLKVCDRDRFRQRRRQEAWFYGSGCDYAAPLKSSVDRHRSIN

SEQ ID NO: 286 - protein, 418 aa, Streptomyces griseus
  REGION 1..418: note = misc_feature - Sg.CrtY_P72451, Streptomyces griseus NCBI 3933
MPTDFDVVIVGAGAAGMSLAYHLCAPDSDVPLSVALVDAPPGPLRAPPRTWCFWEPPGGPYDPVLAASWPRLRVRAADGASTVAQLPRLRYKMLRSDAFEALVEQRFSRAPDLCRMEATASSVRDDPSGVGGEVLTRTACGERILVRGRLVFDSRPSHRLPPARTTLLQHFTGWFVRTERPVFDPGTADLMDFRTPQPARGLSFGYVLPLDPHTALVEYTEFSPAPLDTDGYRRALRHYTHDVLRLGPLQVTAQEHGVIPMTDGRFPHKAGRSVYRIGTAGGATRPSTGYTFAAVQRQSRAVADQLRSGRPLRVPAPYGRRARLMDAVLLRALDSGRVDGADFFHRLFRHIPGERLLSFMDGRSQLHEDLLIGLRTPMVPMLRTVFELPFRTRRARPAAPFPPHRPPPKGAPHDPVAR

SEQ ID NO: 287 - protein, 226 aa, Sulfolobus solfataricus
  REGION 1..226: note = misc_feature - Ss.CrtY_Q97UT9, Sulfolobus solfataricus P2
MEIFKPDYLMIDSMIFFPTLILSFLIKRNYVKLLKSIGIVSPIYLFWDFLATWRNSWTFNPKYVAGIYVIDLPIEEVMFFLVTPFATLLIFDFVMGKVRDKEVKWVPKVVCVAIPLLLLTLPFVYNYSYTFIDLVYLIMSLIVSLLIGRNLLASRNFWIFMGLSYIPFLVFDYFLTSDPVVIYGSHSIVGIRFITIPIEDFIYSFSMITFYTVFYIRGNSLWMKQK

SEQ ID NO: 288 - protein, 367 aa, Vibrio angustum
  REGION 1..367: note = misc_feature - Va.CrtY_Q1ZMY2, Vibrio angustum S14
MKVNVAILGAGCAGLALARCLKEQGFSGSVLLLESRQCYENDRTWCYWAKPDSQWHKMAQYRWQQCLFSSLNDAQHIHYYDSTSYCCLPAENYYDFCLQQLDSANVSLKMEVEVVSVSPTRSQSWLVETTAGNLEADIVIDTRPRPCEAILYQSFLGYEVTLSRPIFAPDTVRLMCNMHGDNQCMRFYYILPFSPWHILIEPTVFHRYPLNPDLLKDDLQSALRAEDIEIESIIRRESAILPMGQTHLEGSVLVKGGIAGGALRASSGYGFLRIQAWALACAQSIITKQKAFSHQQHHLQARMDHLFLNVIKHNPVDGPDYFIRMASQLNGDQFSAFMSDEAGMKEWMQVISALPKWPFIKEVMAGG

SEQ ID NO: 289 - protein, 302 aa, Synechococcus sp.
  REGION 1..302: note = misc_feature - Ss.GGPPS_B1XJV9; Synechococcus sp. PCC 7002
MVVADAHTQGFSLAQYLQEQKTIVETALDQSLVITEPVTIYEAMRYSLLAGGKRLRPILCLAACEMLGGTAAMAMNTACALEMIHTMSLIHDDLPAMDNDDLRRGKPTNHKVYGEDIAILAGDALLSYAFEYVARTPDVPAERLLQVIVRLGQAVGAEGLVGGQVVDLESEGKTDVAVETLNFIHTHKTGALLEVCVTAGAILAGAKPEEVQLLSRYAQNIGLAFQIVDDILDITATAEELGKTAGKDLEAQKVTYPSLWGIEKSQAEAQKLVAEAIASLEPYGEKANPLKALAEYIVNRKN

SEQ ID NO: 290 - protein, 308 aa, Ostreobium quekettii
  REGION 1..308: note = misc_feature - Oq.GGPPS_CAD7703201.1; Ostreobium quekettii
MLETRPPPSTSETETFDFKGYMKEQAALVETALDSAVPLARPETIHESMRYSLLAGGKRVRPVLCLAACELVGGSLENAMPTACALEMLHTMSLIHDDLPSMDNDDLRRGKPTNHKMFGEDMAILAGDALLTFAFEHVACATKGVAADRVLKVITNLGKAVGTRGLVAGQVVDLESEGKVVDLDVLEYIHEHKTAALLEAAVVCGATLGGASDEEIEKLRKYSRNIGLAFQVIDDILDVTQTSEHLGKTAAKDLASDKTTYPKLLGLEKSREVAQHLIDEAVAQLASFNSTRVAPLVGLAHYIGSRTN

SEQ ID NO: 291 - protein, 306 aa, Chlamydomonas debaryana
  REGION 1..306: note = misc_feature - Cd.GGPPS_KAG2491721.1; Chlamydomonas debaryana
MATAEQVEVKPGSFDFNSYMTERAKLVNKALDEAVPQKYPDTLYESMRYSLLAGGKRVRPALCLAACELVGGDIQAALPSACAMEMVHTMSLIHDDLPSMDNDDFRRGRPTNHKVFGEDIAILSGDALLTYSFEHIARATKGVPAERVLRVVMELGKAVGAEGLVAGQVVDIQSEDKEVGLDVLRYIHTSKTAALLEASVVCGAILGGADDGTIEKLRKYSLNIGLAFQVIDDILDVTQTTEQLGKTAAKDLAVNKTTYPKLLGLEKSKQVAEDLITEAIQQLDGFDKARAAPLVALAKYIGYRQN

SEQ ID NO: 292 - protein, 315 aa, Arabidopsis thaliana
  REGION 1..315: note = misc_feature - At.GGPPS_P34802; Arabidopsis thaliana
MSVVTKEDNLRQSEPSSFDFMSYIITKAELVNKALDSAVPLREPLKIHEAMRYSLLAGGKRVRPVLCIAACELVGGEESTAMPAACAVEMIHTMSLIHDDLPCMDNDDLRRGKPTNHKVFGEDVAVLAGDALLSFAFEHLASATSSDVVSPVRVVRAVGELAKAIGTEGLVAGQVVDISSEGLDLNDVGLEHLEFIHLHKTAALLEASAVLGAIVGGGSDDEIERLRKFARCIGLLFQVVDDILDVTKSSKELGKTAGKDLIADKLTYPKIMGLEKSREFAEKLNREARDQLLGFDSDKVAPLLALANYIAYRQN

SEQ ID NO: 293 - protein, 320 aa, Blakeslea trispora; identical to SEQ ID NO: 9
  REGION 1..320: note = misc_feature - Bt.GGPPS_H9CDX4; Blakeslea trispora

SEQ ID NO: 294 - protein, 309 aa, Haematococcus pluvialis
  REGION 1..309: note = misc_feature - Hp.GGPPS_QHF16627.1; Haematococcus pluvialis
MVATAGQVAEVHSAPAFDFEMYMRDRAEMVNKALDAALPSRYPEVLVDSMRYSVLAGGKRVRPALTLAACDLVGGDMATALPTACAMEMIHTMSLIHDDLPAMDNDDFRRGRPTNHKVYGEDIAILAGDALLSFAFEHIARDTKGVPAEAVLKVIMELGRAVGAQGLSAGQAVDIKSEGQEVGLEVLEYIHHHKTAALLEAAVVCGALVGGADTATVEKLRKYALNIGLAFQVIDDILDVTQTTETLGKTAAKDLAVNKTTYPKLLGLEASRKVADDLIREAIAQLDEFEPARKAPMVALAHFIGYRKN

SEQ ID NO: 295 - protein, 312 aa, Cistus creticus
  REGION 1..312: note = misc_feature - Cc.GGPPS_AF492023; Cistus creticus
MSVLSGKDTMKGEEENSGFDFQSYMDQMADSVNQALESAVSLREPLKIHEAMRYSLLAGGKRVRPLLCIAACELVGGDVSVAMPAACAVEMIHTMSLIHDDLPCMDNDDLRRGKPTNHKAFGEDIAVLAGDALLSFAFEHVAVSTVGASPDKIVRAVGELAKAVGKEGLVAGQVVDITSEGLNDVGLDHLEYIHVHKTAVLLEAAVVLGAILGGGTDEEVERLRKFAICIGLLFQVVDDILDVTKSSVELGKTAGKDLVADKVTYPKLMGLEKSREFAEKLRDDAVEQLRVFDQVKAAPLIALAHYIAYRQN

SEQ ID NO: 296 - protein, 313 aa, Brassica napus
  REGION 1..313: note = misc_feature - Bn.GGPPS_XP_013655023.1; Brassica napus
MSSIVRKQDNHMKSSSFDFMSYMARKVETVNRALDLAVPLQEPLKIHEAMRYSLLAGGKRIRPLLCIAACELVGGEASVAMPAACAVEMIHTMSLIHDDLPCIDNDDLRRGKPTNHKVFGENVAVLAGDALLPFAFEHLVTATSPEVSPVRLVRAVGELAKAVGVEGVAAGQVADISSEGLDSNEVGLEHLEFIHLHKTAALLEASAVLGGIVGGGSEDEIEKLRQYARCVGLLFQVVDDILDVTKSSQELGKTAGKDLVADKLTYPKIMGLEKSREFAEKLCRDAREQLLGFVSDRVAPLLALANYIANRQN

SEQ ID NO: 297 - protein, 309 aa, Haematococcus pluvialis
  REGION 1..309: note = misc_feature - Hp.GGPPS_QHF16627.1; Haematococcus pluvialis
MVATAGQVAEVHSAPAFDFEMYMRDRAEMVNKALDAALPSRYPEVLVDSMRYSVLAGGKRVRPALTLAACDLVGGDMATALPTACAMEMIHTMSLIHDDLPAMDNDDFRRGRPTNHKVYGEDIAILAGDALLSFAFEHIARDTKGVPADAVLKVIMELGRAVGAQGLSAGQAVDIKSEGQEVGLEVLEYIHHHKTAALLEAAVVCGALVGGADTATVEKLRKYALNIGLAFQVIDDILDVTQTTETLGKTAAKDLAVNKTTYPKLLGLEASRKVADDLIREAIAQLDEFEPARKAPMVALAHFIGYRKN

SEQ ID NO: 298 - protein, 364 aa, unidentified
  REGION 1..364: note = Ml.GGPPS_QIG55789.1; Melampsora larici-populina
MKPVPSTNGKVDDKVEHHNLSSSSSSSSSSSSSDTKFNISNRYGNFLQRLEALDIWPESNEQILLEPYTYLTNIPGKEIRSMMIDAFNHWLQVPRPALEIIKKIVGQLHTASLLMDDVEDDSDLRRGVPVTHKIYGIPQTINTANYVYFLAYQELTKLKPCLRSDATTDLWSLVNDELLQLHRGQGMDLYWRDSLTCPTEEEYLQMVNNKTGGLFRIAIKLMIALSPIPETPDYLPLVNLVGIIFQIRDDLLNLSSVYTKNKGFCEDLTEGKFSFPIVHSIRSDSTNHQLMNILRQKPTDIGTKAFAVSYMKDRTKSLEYTRGVLICLEEQAIEEVTRLGGNPALESIFELMHVLPSPPATDQN

SEQ ID NO: 299 - protein, 371 aa, unidentified
  REGION 1..371: note = Cg.GGPPS_WP_011014931.1; Corynebacterium glutamicum ATCC 13032
MKDVSLSSFDAHDLDLDKFPEVVRDRLTQFLDAQELTIADIGAPVTDAVAHLRSFVLNGGKRIRPLYAWAGFLAAQGHKNSSEKLESVLDAAASLEFIQACALIHDDIIDSSDTRRGAPTVHRAVEADHRANNFEGDPEHFGVSVSILAGDMALVWAEDMLQDSGLSAEALARTRDAWRGMRTEVIGGQLLDIYLESHANESVELADSVNRFKTAAYTIARPLHLGASIAGGSPQLIDALLHYGHDIGIAFQLRDDLLGVFGDPAITGKPAGDDIREGKRTVLLALALQRADKQSPEAATAIRAGVGKVTSPEDIAVITEHIRATGAEEEVEQRISQLTESGLAHLDDVDIPDEVRAQLRALAIRSTERRM

SEQ ID NO: 300 - protein, 315 aa, Antirrhinum majus
  REGION 1..315: note = misc_feature - Am.GGPPS_AY534687; Antirrhinum majus
MILTKNPQESSQKTSKDPTFTLDFKTYMLEKASSVNKALEQAVLLKEPLKIHESMRYSLLAGGKRVRPMLCIAACELVGGLESTAMPSACAVEMIHTMSLIHDDLPCMDNDDLRRGKPTNHKIYGEDVAVLAGDALLAFSFEHVAKSTKGVSSDRIVRVIGELAKCIGSEGLVAGQVVDISSEGMTEVGLEHLEFIHVHKTAALLEASVVLGAIVGGADDEDVEKLRKFARCIGLLFQVVDDILDVTKSSQELGKTAGKDLVADKTTYPKLLGIEKSREFAEKLNREAQEQLEGFDSVKAAPLIALANYIAYRDN

SEQ ID NO: 301 - protein, 332 aa, Candida albicans
  REGION 1..332: note = misc_feature - Ca.GGPPS_XP_711426.1; Candida albicans
MQLDINKLIQPTEAYVPESSDPIVQPYTYISETPSNNQNVRTRFLHAFNDLFYKISDESLLARISEIISVFHNSSLLIDDIEDDSKYRRGVPVAHVKYGIPTTINCGNLMYFVALQQAQQLSGPHGSMEVKFKSSQILIDELLNLHRGQGLDIYWRDYLKKLEKLPDVEEYLEMIKDKTGGLFRLAIKLLSLYSDVKENDQLISLANLMGILYQVRDDYLNLVDAKYSAMKGTTCEDLIEGKLSLPILHCLRTTKDSPVHKILYDYDSSSDRVSQNSLIDLSLSFMKNESKSLEYTLNLIKVLEKKLRQLILKYPESENSALLKIVERLCDL

SEQ ID NO: 302 - protein, 325 aa, Methanoregula boonei
  REGION 1..325: note = misc_feature - Mb.GGPPS_WP_012107783.1; Methanoregula boonei 6A8
MEADMSDLSAYLKSVAQQIDGMIEKNFTHAGGELDRASAHLLSAGGKRLRPAVVMLSADAIRHGSSKDVMPAALALEVTHTFTLIHDDIMDGDSLRRGVPTVHTKWDMPTGILAGDVLYARAFEFICQSKADEGPKVQAVALLARACADICEGQHQDMSFEHRADVTEEEYMAMVAKKTGVLYAAAAAIGGTLAGGNPEQIRALYQFGLNTGIAFQIQDDLIDLLTPTEKSGKDQGSDLREGKQTLVMIIARQKGVDLLKYRHELSPADIKAAIQELTDAGVIDAVKKKAADLVADSNRLLMVLPPTKERQLIMDVGEFFVTRSF

SEQ ID NO: 303 - protein, 330 aa, Thermus thermophilus
  REGION 1..330: note = misc_feature - Tt.GGPPS_WP_011227641.1; Thermus thermophilus
MVPAPEAIRQALQERLLARLDHPDPLYRDLLQDYPRRGGKMLRGLLTVYSALAHGAPLEAGLEAATALELFQNWVLVHDDIEDGSEERRGRPALHRLHPMPLALNAGDAMHAEMWGLLAEGLARGLFPPEVLLEFHEVVRRTAYGQHLDLLWTLGGTFDLRPEDYFRMVAHKAAYYTAVAPLRLGALLAGKTPPAAYEEGGLRLGTAFQIVDDVLNLEGGEAYGKERAGDLYEGKRTLILLRFLEEAPPEERARALALLALPREAKPEAEVGWLLERLLASRALAWAKAEAKRLQAEGLALLEAAFQDLPGKEALDHLRGLLAALVERRA

SEQ ID NO: 304 - protein, 314 aa, Raphanus sativus
  REGION 1..314: note = misc_feature - Rs.GGPPS_XP_018455142.1; Raphanus sativus
MEFLDSSTTQAENKEDDTTEFDFKPYMIRKAESINRALDKAIPLVEPLDIHRAMRYALLAGGKRVRPILCLAVCELVGGEERLAVPAACAVEMIHTMSLIKDDLPCMDNDDLRRGKPTTHKVFGESVAVLSGGALLALAFEHLTEADVSPERMVRAVKELARSIGTKGLVAGQAMDLSSEGLDRNDVGLEELEFIHVHKTGSLLEASAVIGAVIGGGSEEEVERVRKFARCVGLLFQVVDDILDGTKSSEELGKTAGKDQVAGKLTYPKVLGLEKSKEFVERLRRDAREHLKGFDSDKAKPLVALADFIANRDN

SEQ ID NO: 305 - protein, 321 aa, Arabidopsis lyrata
  REGION 1..321: note = misc_feature - Al.GGPPS_XP_020888857.1; Arabidopsis lyrata subsp. lyrata
MALTSQGARDMIPPEGKSDDRNSAFDFKSYMINKAKSVNAALDISVPLREPLTVQEAVRYSLLAGGKRVRPMLCIASCELVGGNEATAMPAACAVEMIHTSSLIHDDLPCMDNADLRRGKPTNHKVFGEDMAVLAGDALLALAFEHMTVVSSGLVPPERMIRAVAELARAIGTTGLVAGQMIDLASERLNPHDAGLERLEFIHLHKTAALLEAAAVLGVIMGGGTEEEIEKLRKYARCIGLLFQVVDDILDVTESTEELGKTAGKDLMAGKLTYPRLIGLERSREVAEKLRKEAEEQVLGFDSENSAPLVALASYIACRHN

SEQ ID NO: 306 - protein, 315 aa, Arabidopsis thaliana
  REGION 1..315: note = misc_feature - At.GGPPS_P34802_L155M; Arabidopsis thaliana
MSVVTKEDNLRQSEPSSFDFMSYIITKAELVNKALDSAVPLREPLKIHEAMRYSLLAGGKRVRPVLCIAACELVGGEESTAMPAACAVEMIHTMSMIHDDLPCMDNDDLRRGKPTNHKVFGEDVAVLAGDALLSFAFEHLASATSSDVVSPVRVVRAVGELAKAIGTEGLVAGQVVDISSEGLDLNDVGLEHLEFIHLHKTAALLEASAVLGAIVGGGSDDEIERLRKFARCIGLLFQVVDDILDVTKSSKELGKTAGKDLIADKLTYPKIMGLEKSREFAEKLNREARDQLLGFDSDKVAPLLALANYIAYRQN

SEQ ID NO: 307 - protein, 347 aa, Lodderomyces elongisporus
  REGION 1..347: note = misc_feature - Le.GGPPS_XP_001524890.1; Lodderomyces elongisporus NRRL YB-4239
MDYKKVVNSLAQPTVVDPAVIEPYKYIRKLASNNNNVRSRFINAFNETFFHITDQEVLQLINEVIEILHNASLLIDDVEDSSNFRRGYPSAHMKYGVPLTINCGNLMYFEAIEKVYNLWRLKLGKEDTTLATLALVWEIVMPEMKNFHKGQGLDIYWRDNLPDLESLPSVVDYCQMAKDKTGGLFRLAVRLLRLFSSGSTHLDSEYNGVFESIANCLGVIYQIRDDYLNIVDERYSHMKGIRGEDLIEGKLSFPILVSLTKDFNQKHTEISSLSPLYELLYNHRSTEERLRRRDLHDQAIEQLESPDIVSETNRLLQELKAQACRLIMENARDANPLMIMVDTLCTI

SEQ ID NO: 308 - protein, 332 aa, Chlorobium tepidum
  REGION 1..332: note = misc_feature - Ct.GGPPS_WP_010931948.1; Chlorobium tepidum TLS
MSSPITQAQVESKYRQYHAKINEALAACFPKEKPATLYDPARYILEGKGKRIRPFLTLLAAEAVSGKSDNALGVALGIEVLHNFTLMHDDIMDQADLRHGRPTVHKQWNVNAAILSGDMMIAYAYELALKAISSRHAEIIHIFNDANITICEGQALDMELEQRKDVTIADYLDMISKKTGRLISAALEAGGVAGDGTPEQIAALVTFGEKIGRAFQIQDDYLDIMAGDGKSGKVPGGDVINGKKTWLLLRSLELAEGADRELLQSIFDNNGTSPDNVPAVKAIFEKCGVLNETRAKINEDTEAALAALDALPFEEGRGYLRGFANILMKRDF

SEQ ID NO: 309 - protein, 311 aa, Picea abies
  REGION 1..311: note = misc_feature - Pa.GGPPS_B1A9L0; Picea abies
MELVNESENKDEEKPIAFEFKKYMFSKANAINEALDKCVSLRHPEKIHEAMRYSLLAGGKRVRPILCIASCELVGGSEESVMPSACAVEMIHTMSLIHDDLPCMDNDDLRRGKPTNHKVFGEDVAVLAGDALLAFAFEHIACSTKGVEAEKVLRVIWELGRAIGSEGLVAGQVVDLSSGGLSNVGLDLLEYIHVHKTAALLEGSVVIGAIIGGGSDEEIERLRRFARCIGLMFQVVDDILDVTKSSQELGKTACKDLVADKLTYPKLLGLEKSREFAQELNKQAMDQISVFDVEKAKPLICLADYIAHRQN

SEQ ID NO: 310 - protein, 272 aa, Thermotoga maritima
  REGION 1..272: note = misc_feature - Tm.GGPPS_WP_004082783.1; Thermotoga maritima MSB8
MKKEKVEERIREILRPGWDLLTEEAMLYSATVGGKRIRPLLVLTLGEDLGVEEEKLLDVAVAVELFHTASLIHDDLPPIDNADFRRGKPSCHRTYGEDIALLAGDGLFFLAFSQISKIGNSKIFEEFSETAYKLLLGEAMDVEFERRKMEVSQEMVERMYAFKTGALFAFCFSAPFILKGKDHTKMKLLGEKFGVAFQIYDDLKDILGSFEKVGKDLGKDTEKVTLVKKVGIQKAREMADKYYEEVLKGIESEGLFRTLFLLKELKQMVEER

SEQ ID NO: 311 - protein, 335 aa, unidentified
  REGION 1..335: note = Mb.GGPPS_O06428; Mycobacterium bovis AF2122/97
MRTPATVVAGVDLGDAVFAAAVRAGVARVEQLMDTELRQADEVMSDSLLHLFNAGGKRFRPLFTVLSAQIGPQPDAAAVTVAGAVIEMIHLATLYHDDVMDEAQVRRGAPSANAQWGNNVAILAGDYLLATASRLVARLGPEAVRIIADTFAQLVTGQMRETRGTSENVDSIEQYLKVVQEKTGSLIGAAGRLGGMFSGATDEQVERLSRLGGVVGTAFQIADDIIDIDSESDESGKLPGTDVREGVHTLPMLYALRESGPDCARLRALLNGPVDDDAEVREALTLLRASPGMARAKDVLAQYAAQARHELALLPDVPGRRALAALVDYTVSRHG

SEQ ID NO: 312 - protein, 310 aa, Picea abies
  REGION 1..310: note = misc_feature - Pa.GGPPS_B1A9K9; Picea abies
MQLANLREEVKEVIKFDFKEYLLSKAMAVNEALDRAVQLRYPEKIHEAMRYSLLAGGKRVRPVLCIAACELVGGTEKLAMPTACAMEMIHTMSLIHDDLPCMDNDDFRRGKPTNHKVFGEGTAVLAGDALLSFAFEHIAVSTSKSVGSDRILRVVSELGRTIGSQGLVGGQVADITSEGNASVDLDTLEWIHIHKTAVLLECSVVCGAIISGASENEIERVKRYARSVGLLFQVVDDILDVTKSSKELGKTAGKDLISDKATYPKLMGLEKAKQFAVELLDRAKENLSCFDPMKAAPLLGLADYIAFRQN

SEQ ID NO: 313 - protein, 314 aa, Eutrema salsugineum
  REGION 1..314: note = misc_feature - Es.GGPPS_XP_006406415.1; Eutrema salsugineum
MHFNIQSLTSVTRNKFDFMSYMVNKAKSVNKALDEAVPLCEPVLKIREAMRYTLLSGGKRVRPMLCLAACELVGGQESTAMPAACAIEMLHASSLIQDDLPCMDDDNLRRGKPTNHKVFGEGIAVLTADALIALAVKKMATSSTSSPEKVLRAILEMAKAVGTEGLVAGQAADLAGEGMSLEENDDVGLKHLEFIHIHKTAAMLEAAAVIGGIMGGGSDEEIERLRSYARCIGLMFQVVDDVLDVTKTSEELGKTAGKDLIAGKLTYPKVMGVEKSKEYAERLNREAREHLQGFDSDKVAPLLFLADYIVNRQN

SEQ ID NO: 314 - protein, 365 aa, Melampsora lini
  REGION 1..365: note = misc_feature - Ml.GGPPS_QIG55792.1; Melampsora lini
MKPLPSTNGKVNGNGKHHDSSLSSTSSTSSSSSSDTQFNISDRYGDFLHRLDTLDTWPKSNEQILLEPYTYLNNIPGKEIRSMMIDAFNHWLQIPRPALEIIKKIVGQLHTASLLMDDVEDDSDLRRGVPVTHKIYGIPQTINTANYVYFLAYQELSKLKPCLSSNASTDLWSLVNDELLQLHRGQGMDLYWRDSLTCPTEEEYLQMVNNKTGGLFRIAIKLMIALSPLTETPDYLPLVNLVGIIFQIRDDLLNLSSVYTKNKGFCEDLTEGKFSFPIVHSIRADSSNHQLMNILRQKPTDIGTKTFAVSYMKDQTKSLQYTREVLTCLEEQAIEEVTRLGGNPALESIFELMHVLPSPPATDQH

SEQ ID NO: 315 - protein, 334 aa, Pyrobaculum calidifontis
  REGION 1..334: note = misc_feature - Pc.GGPPS_WP_011848845.1; Pyrobaculum calidifontis JCM 11548
MDVVSRLHQKYGAEVEKALVRYLSIGLAEDFREAVLYQVKTGGKRLRPLLTLAAAEAVSGQWRPALPAAAIVELIHNYSLIYDDIIDRGDVRRGLPTVRKAFGDNAAILVGIWYREAIEEAVLDTPKPTLFAKEVAEVIKAIDEGERLDILFEAAGRSDPYFVQARWREVTLDDYIKMVSLKTGALIAAAAKWGVLSVSDDRGLAEAAWNFGMAAGVAFQIIDDVLDIYGDPKKFGKEIGKDIKEHKRGNAVVAVALSHLGEGERRRLLEILAREVVEEADVREAVALLDSVGAREEALRLAARYREEAERHLAKIPNNGTLKELLDFIVAREY

SEQ ID NO: 316 - protein, 382 aa, unidentified
  REGION 1..382: note = Cg.GGPPS_WP_011013776.1; Corynebacterium glutamicum ATCC 13032
MRDNWPCDTRMDNGMTITTEHSTHPDLDFNDEIYRELNRICASLSQQCSTYQPEFRTCLDAAFQALRGGKLIRPRMLLGLYNTLVDDDIEVKLNTVLQVAVALELLHFSLLVHDDVIDGDLYRRGKLNFIGQILMHRTPESFAQIQRDPEHLDWAQSNGLLMGNLFLAATHQIFARLDLPHHQRVRLLDLLNHTINDTIVGEFLDVGLSSKAISPNMDIALEMSRLKTATYTFELPMRAAAILAELPQEIETKIGEIGTNLGIAYQLQDDYLSTFGDAAEHGKDAFSDLREGKETTIIAFARDTAKWTDIQDNFGSADLSTSQAERIQHLLIQCGAKNHSLNAISDHLNICRSMIKTLSPQVDPKAQNLLLKQVEQLASRKS

SEQ ID NO: 317 - protein, 313 aa, Litsea cubeba
  REGION 1..313: note = misc_feature - Lc.GGPPS_ART33318.1; Litsea cubeba
MVIEGEKENDSSYSETTPPFDLKTYMRQKGNSVNRALEEAVPLRSPEALYEPMRYSLFAGGKRVRPLLCISACELVGGNEATAMPSACAAEMIHTMSLMHDDLPYMDDDDLRRGKPTAHKAFGETAAVLSGDVMLLLAFEHVAAATPGAPPDRVARAVGELARAAGRDGLVAGQIVDVCSEGSGEVGLERLEYIHLHKTAALLEGAVVAGGMLGGGTEEEIGRLRRFARCIGLLFQVVDDILDVTKSSTELGKTAGKDVAAEKVTYPKMLGLEGSRELAEELRKEAKEQLMGFDPEKAMPLVALTDFIACRNN

SEQ ID NO: 318 - protein, 301 aa, Lactococcus lactis
  REGION 1..301: note = misc_feature - Ll.GGPPS_WP_011835496.1; Lactococcus lactis subsp. cremoris MG1363
MRLISKEHKIFGIIRVMDTKILKLEHFLTEFYESAEFPKGLAEPAKYSLMAGGKRIRPLLFLNLLEAFDLEVTTAHYQVAAALEMIHTGSLIHDDLPAMDNDDYRRGKLTNHKKFDEATAILAGDTLFFDPFYVLSSSDLSAQTIVSLTRELAYASGSYGMVAGQILDMAGEGKDLTLTQIEQIHRLKTGRLLTFPFVAAGIVAQKTNQEIEKLRVVGQILGLAFQIRDDILDVTATFAELGKTPGKDVLEEKSTYVAHLGLEGAKISLTDKLSEVKKLLTELNVTDSSEIFKIIEQLEVE

SEQ ID NO: 319 - protein, 309 aa, Legionella pneumophila
  REGION 1..309: note = misc_feature - Lp.GGPPS_WP_010948036.1; Legionella pneumophila subsp. pneumophila str. Philadelphia 1
MVLIPTLNSTQMNKQVIDKYTQRHELYLEQLLNEIIIPAPQIRSALHYALFSGGKRIRPILVYLAGDLIDVDQGVLDIIAAALELTHCYSLIHDDLPAMDNDDLRRGKPSCHKAFDEATAILVGDGMQALAIEVLLMRLSPLLPAAQVVAITQVLVNASGISGMVSGQSLDLSELAKSSVTEEQLREIHLLKTGKLILACFEMVLAAQHEVSEQIKSALRTYGKHIGLVFQMQDDYLDLYAPTQILGKGRSSDQANQKTTFATLFNKQQLEEEIAVHYQIAMDSLRLFGSKAAALIELTKQLQNRSNLS

SEQ ID NO: 320 - protein, 315 aa, Nicotiana sylvestris
  REGION 1..315: note = misc_feature - Ns.GGPPS_XP_009784772.1; Nicotiana sylvestris
MEAEVIKSENTFREAGKTKTVFDFKSYMYQKINSVNRALDAAVPLREPLKFHEAMRYSLLSEGKRVCPVLCIAACELVGGQESTAMPAACGMEMIHAMCMMHDDLPCMDNDNLRRGKLANHKVFGENVTVLAGYSLVALAFEHMATATKGVHPKTMVRAIGELARLIGPEGAAAGQVLDLLSGGESDTGLEELEYIHRHKTADFAEAAVIVGAMVGGATDEEINRLRKFSQCIGLLFQVVDDILDVTKSSEQLGKTAGKDLLANKLTYPKMIGIEKSKEYAQNLSKEAKEQLVGFDPEKAAPLLAMADFVLHRQK

SEQ ID NO: 321 - protein, 310 aa, Perilla frutescens
  REGION 1..310: note = misc_feature - Pf.GGPPS_KAH6803897.1; Perilla frutescens var. frutescens
MIRFISNKIHQNDQFDFKAYMLEKITAVNQALDAALPLQEPTKLHEAMRYSLLSGGKRICPIVCLAACRLVGGNEAAAMPSACALEMIHAMSLMHDDLPCMDNDDLRRGRPSSHKVFGEGAAVLAGYALIATAFDHIATATKGVAEGRIVRVVGELARLIGPEGVVAGQVADLQCGGRGRKDIGLEQLEYIHLQKTAASVEASAVAGAIIGGASEEEIEKLRKYSRRAGLLFQVVDDILDVTKSSEELGKTAGKDLAAEKATYPKLIGVEKSRELAEQLKREAHQQLLGFDGVRAAPLIALLHFIAYRDK

SEQ ID NO: 322 - protein, 293 aa, Paracoccus haeundaensis
  REGION 1..293: note = misc_feature - Ph.GGPPS_AAY28422.1; Paracoccus haeundaensis
MRRDVNPIHATLLQTRLEEIAQGFGAVSQPLGAAMSHGALSSGRRFRGMLMLLAAEASGGVCDTIVDAACAVEMVHAASLIFDDLPCMDDAGLRRGRPATHVAHGESRAVLGGIALITEAMALLAGARGASGTVRAQLVRILSRSLGPQGLCAGQDLDLHAAKNGAGVEQEQDLKTGVLFIAGLEMLAVIKEFDAEEQTQMIDFGRQLGRVFQSYDDLLDVVGDQAALGKDTGRDAAAPGPRRGLLAVSDLQNVSRHYEASRAQLDAMLRSKRLQAPEIAALLERVLPYAARA

SEQ ID NO: 323 - protein, 291 aa, Streptococcus pneumoniae
  REGION 1..291: note = misc_feature - Sp.GGPPS_ZP_01823217.1; Streptococcus pneumoniae
MKKQEKLALVESVLEDFYGDQQFASSLRESVLYSIHAGGKRIRPFLLLEVLEALQVAIKPAHAQVATALEMIHTGSLIHDDLPAMDDDDYRRGRLTNHKKFGEAMAILAGDALFLDPYALIAQADLPSQIKVDLIASLSLASGSLGMVAGQVLDMEGEHQHLSLEELQTIHANKTGKLLAYPFQAAAIIAELSPEMQVKLKTVGELIGLAFQVRDDVLDVTASFEEIGKTPQKDLQAEKSTYPALLGLEESIVFCNQTLDQANEKLEEIAQQVPFETESIVSVVESLRING

SEQ ID NO: 324 - protein, 299 aa, Shigella sonnei
  REGION 1..299: note = misc_feature - Ss.GGPPS_NP_706309.1; Shigella sonnei
MDFPQQLEACVKQANQALSRFIAPLPFQNTPVVETMQYGALLGGKRLRPFLVYATGHMFGVSTNTLDAPAAAVECIHAYSLIHDDLPAMDDDDLRRGLPTCHVKFGEANAILAGDALQTLAFSILSDANMPEVSDRDRISMISELASASGIAGMCGGQALDLDAEGKHVPLDALERIHRHKTGALIRAAVRLGALSAGDKGRRALPVLDKYAESIGLAFQVQDDILDVVGDTATLGKRQGADQQLGKSTYPALLGLEQARKKARDLIDDARQALKQLAEQSLDTSALEALADYIIQRNK

SEQ ID NO: 325 - protein, 292 aa, unidentified
  REGION 1..292: note = Um.GGPPS_AAR37858.1; uncultured marine proteobacterium
MENGLLDCEQYLEEAMAEHATAQCPPLLAQALNYAVFPGGARVRPKICKAVALANNSSDVGLANAAASAIELLHCASLVHDDLPCFDDATQRRGKPSVHAKFGERIAVLTGDALIVAAFQTLATHAIHAVRTERVPLVTAIVARGVGAPHGICAGQAWECERSVDLSRYHRAKTGALFVAATCAGAAAAGVDPGPWVNLGASIGEAYQVADDIKDAISDPETLGKPTGIDVKLDRPSAVRELGLDGAVTRLKQCLEAGLDSMPACAGQDLLQKIVRAQASRFVPEKIAQVAA

SEQ ID NO: 326 - protein, 309 aa, Vigna radiata
  REGION 1..309: note = misc_feature - Vr.GGPPS_XP_022634843.1; Vigna radiata var. radiata
MLSPPDTVRREEIGFDFKAYMQHKINTVNRALDAAIELKEPKNIHEAMRYTLLAGGKKIRPVLCIAACELVGGTDYAAVPAACAVEMVHAMSLIHDDLPSIDNDDLRRGKPACHKVFGENVAILAGDALQAYAFEHLAASTKGVSPTRVVRAIVELAKSVGAEGVAGGQVMDVSSEGLSSEVGLERVELIHLKKSAALLEGSVVLGAIVGGGSDEEVERLRMFGRCVGLMGQIVDDIVDVTKSTEELGKTAGKDLVVDKLTYPKVFGMHKSKEIAQKLVEDSTEILVGFDNPKVAPLVALTNYIAGKIN

SEQ ID NO: 327 - protein, 332 aa, Nocardia brasiliensis
  REGION 1..332: note = misc_feature - Nb.GGPPS_B1Q2P2; Nocardia brasiliensis
MKPGYIDSRTEEFDPAPALRHYLAGTAAGDGDRLDAVTRYAVQPPGKLVRPVLLVESAAAVGADPADLVPIAVALEYLHVATLVHDDIIDGDLLRRGRPTVHARYGRDEAIVAGDALLLDTFRTLTEAAELGFPPAAVLEAVRIVAVAGLDLCRGQAMEAGLAGDLYCGMARYRTMAGLKTGALFIAACAAGAVLGGGSGEEVRALRSFADHLGCAFQMRDDLLPYLADTSRAGKSSVTDIVNQRPTFPVLVGIELADTTQCWRIEQSLSGRLPADEAHRLLRQVLTETGALAEASARVAAEVAAARADLAPFGVRGERLIALADSMIGRTW